(19) United States
(12) Patent Application Publication
(10) Pub. No.: US 2023/0094471 A1
(43) Pub. Date: Mar. 30, 2023

(54) CYTOTOXIC BIS-BENZODIAZEPINE DERIVATIVES AND CONJUGATES THEREOF WITH CELL-BINDING AGENTS FOR INHIBITING ABNORMAL CELL GROWTH OR FOR TREATING PROLIFERATIVE DISEASES

(71) Applicant: IMMUNOGEN, INC., Waltham, MA (US)

(72) Inventors: Ravi V.J. Chari, Newton, MA (US); Michael Louis Miller, Framingham, MA (US); Manami Shizuka, Belmont, MA (US); Katie Elizabeth Archer, Boston, MA (US); Emily Elizabeth Reid, Sharon, MA (US)

(73) Assignee: IMMUNOGEN, INC., Waltham, MA (US)

(21) Appl. No.: 17/599,347

(22) PCT Filed: Mar. 27, 2020
(86) PCT No.: PCT/US2020/025341

§ 371 Date: Sep. 28, 2021

Related U.S. Application Data
(60) Provisional application No. 62/825,954, filed on Mar. 29, 2019.

(51) Int. Cl. A61K 47/55; A61K 47/54; A61K 47/65; A61K 47/68

(52) U.S. Cl. CPC A61K 47/552; A61K 47/545; A61K 47/6851; A61K 47/6849; A61K 47/65

(57) ABSTRACT

The invention relates to benzodiazepine derivatives with antiproliferative activity and more specifically to benzodiazepine compounds of formulae (I), (II), (TI) and (T2). The invention also provides conjugates of the benzodiazepine compounds linked to a cell-binding agent. The invention further provides compositions and methods for inhibiting abnormal cell growth or treating a proliferative disorder in a mammal using the compounds or conjugates of the invention.

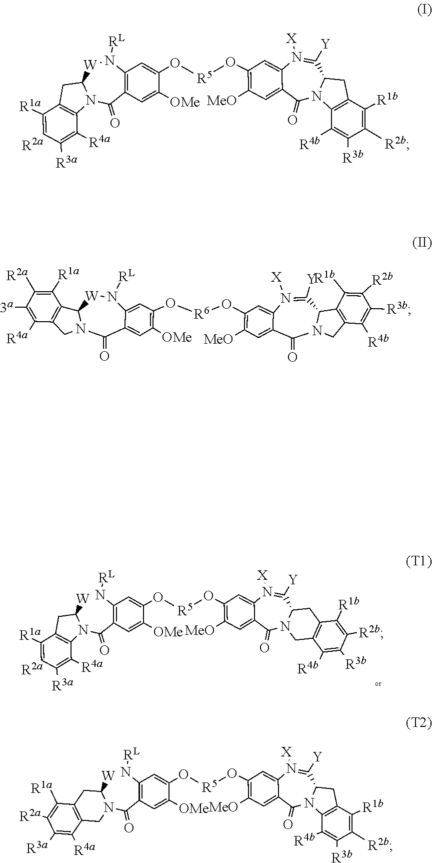

### RELATED APPLICATIONS

This application claims the benefit of the filing date under 35 U.S.C. § 119(e), of U.S. Provisional Application No. 62/825,954, filed on Mar. 29, 2019, the entire content of which is incorporated herein by reference.

### FIELD OF THE INVENTION

The present invention relates to novel cytotoxic compounds, and cytotoxic conjugates comprising these cytotoxic compounds and cell-binding agents. More specifically, this invention relates to novel benzodiazepine compounds, derivatives thereof, intermediates thereof, conjugates thereof, and pharmaceutically acceptable salts thereof, which are useful as medicaments, in particular as anti-proliferative agents.

### BACKGROUND OF THE INVENTION

Benzodiazepine derivatives are useful compounds for treating various disorders, and include medicaments such as, antiepileptics (imidazo [2,1-b][1,3,5] benzothiadiazepines, U.S. Pat. Nos. 4,444,688; 4,062,852), antibacterials (pyrimido[1,2-c][1,3,5]benzothiadiazepines, GB 1476684), diuretics and hypotensives (pyrrolo(1,2-b)[1,2,5]benzothiadiazepine 5,5 dioxide, U.S. Pat. No. 3,506,646), hypolipidemics (WO 03091232), anti-depressants (U.S. Pat. No. 3,453,266); osteoporosis (JP 2138272).

It has been shown in animal tumor models that benzodiazepine derivatives, such as pyrrolobenzodiazepines (PBDs), act as anti-tumor agents (N-2-imidazolyl alkyl substituted 1,2,5-benzothiadiazepine-1,1-dioxide, U.S. Pat. No. 6,156,746), benzo-pyrido or dipyrido thiadiazepine (WO 2004/069843), pyrrolo [1,2-b] [1,2,5] benzothiadiazepines and pyrrolo[1,2-b][1,2,5] benzodiazepine derivatives (WO2007/015280), tomaymycin derivatives (e.g., pyrrolo[1,4]benzodiazepines), such as those described in WO 00/12508, WO2005/085260, WO2007/085930, and EP 2019104. Benzodiazepines are also known to affect cell growth and differentiation (Kamal A., et al., Bioorg. Med. Chem., 2008 Aug. 15; 16(16):7804-10 (and references cited therein); Kumar R, Mini Rev Med Chem. 2003 June; 3(4):323-39 (and references cited therein); Bednarski J. J., et al., 2004; Sutter A. P, et al., 2002; Blatt N B, et al., 2002), Kamal A. et al., Current Med. Chem., 2002; 2; 215-254, Wang J-J., J. Med. Chem., 2206; 49:1442-1449, Alley M. C. et al., Cancer Res. 2004; 64:6700-6706, Pepper C. J., Cancer Res 2004; 74:6750-6755, Thurston D. E. and Bose D. S., Chem. Rev., 1994; 94:433-465; and Tozuka, Z., et al., Journal of Antibiotics, (1983) 36; 1699-1708. The general structure of PBDs is described in US Publication Number 20070072846. The PBDs differ in the number, type and position of substituents, in both their aromatic A rings and pyrrolo C rings, and in the degree of saturation of the C ring. Their ability to form an adduct in the minor groove and crosslink DNA enables them to interfere with DNA processing, hence their potential for use as antiproliferative agents.

The first pyrrolobenzodiazepine to enter the clinic, SJG-136 (NSC 694501) is a potent cytotoxic agent that causes DNA inter-strand crosslinks (S. G Gregson et al., 2001, J. Med. Chem., 44: 737-748; M. C. Alley et al., 2004, Cancer Res., 64: 6700-6706; J. A. Hartley et al., 2004, Cancer Res., 64: 6693-6699; C. Martin et al., 2005, Biochemistry., 44: 4135-4147; S. Arnould et al., 2006, Mol. Cancer Ther., 5: 1602-1509). Results from a Phase I clinical evaluation of SJG-136 revealed that this drug was toxic at extremely low doses (maximum tolerated dose of 45 μg/m², and several adverse effects were noted, including vascular leak syndrome, peripheral edema, liver toxicity and fatigue. DNA damage was noted at all doses in circulating lymphocytes (D. Hochhauser et al., 2009, Clin. Cancer Res., 15: 2140-2147).

Thus, there exists a need for improved benzodiazepine derivatives that are less toxic and still therapeutically active for treating a variety of proliferative diseases, such as cancer.

### Summary of the Invention

The present invention provides novel benzodiazepine dimer compounds and cell-binding agent conjugates thereof. In some embodiments, the benzodiazepine dimer compounds have an imine-reduced (i.e., a single bond between N and C atoms) benzodiazepine as one of the monomers. The dimer compounds of the present invention are covalently linked to the cell-binding agent through a self-immolative linker at the N-10 amine of the reduced benzodiazepine monomer, which can lead to improved metabolism, potency, tolerability and/or solubility of the corresponding cell-binding agent conjugates.

In particular, the dimer compound of the present invention has an imine benzodiazepine as the other monomer, which can be modified with an imine reactive reagent (e.g., sodium bisulfite) to yield a modified (e.g., sulfonated) dimer compound (e.g., a compound described herein, or a pharmaceutically acceptable salt thereof, in which the double line [symbol] between N and C represents a single bond, X is H and Y is —OH or —SO₃H, preferably Y is —SO₃H) with an increased solubility in aqueous solution. As the antibody conjugation reaction is generally carried out in an anqueous solution or a mixture of an aqueous solution and an organic co-solvent, the increase in solubility of the dimer compound can improve the conjugation yield and result in a higher DAR and/or monomer percentage of the resulting conjugate. By comparison, a comparator benzodiazepine dimer compound having a self-immolative linker at the N-10 position of the imine benzodiazepine monomer is difficult to be conjugated to an antibody, at least in part due to its low solubility in an aqueous solution. In addition, the antibody conjugate of the comparator compound has lower DAR and monomer percentage as compared to the conjugates of the present invention (Example 47).

In a first aspect, the present invention is directed to a cytotoxic compound represented by the following formula:

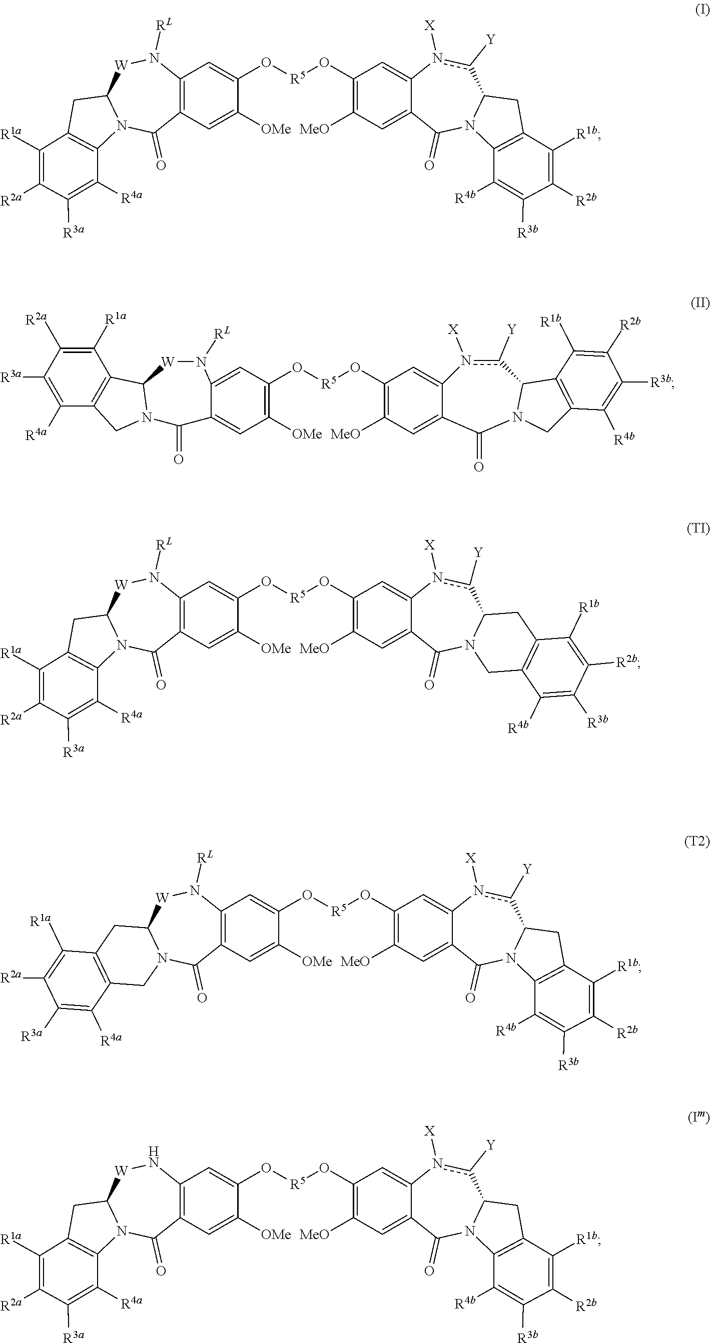

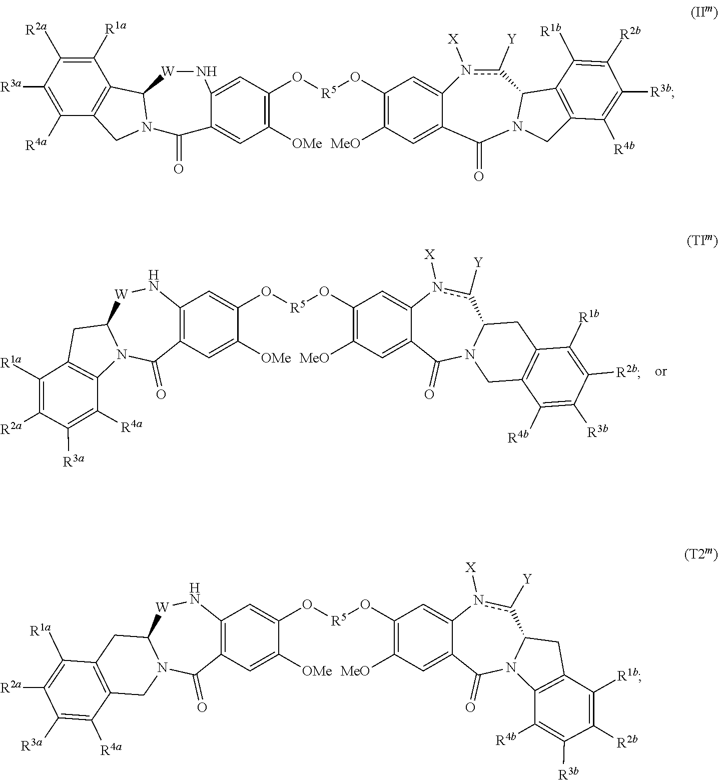

or a pharmaceutically acceptable salt thereof, wherein:

the double line [symbol] between N and C represents a single bond or a double bond, provided that when it is a double bond X is absent and Y is H, or a C₁₋₄alkyl, and when it is a single bond, X is H and Y is —OH or —SO₃H;

W is —C(═O)— or —C(Y′)—;

Y′ is H or C₁₋₄alkyl;

R^{1a}, R^{2a}, R^{3a}, R^{4a}, R^{1b}, R^{2b}, R^{3b} and R^{4b} are each independently selected from the group consisting of H, a C₁₋₁₀alkyl, —(OCH₂CH₂)ₙOR^{c}, halogen, —NH(C═NH)NH₂, —OR, —NR′R″, —NO₂, —NR′COR″, —SR, —SOR′, —SO₂R′, —SO₃H, —OSO₃H, —SO₂NR′R″, —CN, —N₃, —COR′, —OCOR′, and —OCONR′R″;

R^{c} is H or a C₁₋₄alkyl;

n is an integer from 1 to 24;

R, for each occurrence, is independently selected from the group consisting of H, —(CH₂CH₂O)ₙ—R^{c}, C₁₋₁₀alkyl, a C₃₋₈cycloalkyl, a 6- to 18-membered aryl, a 5- to 18-membered heteroaryl ring containing one or more heteroatoms independently selected from N, O and S, or a 3- to 18-membered heterocyclic ring containing 1 to 6 heteroatoms independently selected from O, S, N and P;

R′ and R″ are each independently selected from —H, —OH, —OR, —NHR, —NR₂, —COR, a C₁₋₁₀alkyl, a-(CH₂CH₂O)ₙ—R^{c}, and a 3- to 18-membered heterocyclic ring having 1 to 6 heteroatoms independently selected from O, S, N and P;

R⁵ is a C₃₋₁₂alkylene, which chain can be interrupted by one or more groups selected from —O—, —S—, —NH—, —NMe-, benzene ring, a 4 to 7-membered heteroaryl ring and a 4 to 7-membered heterocyclic ring, wherein the benzene, the 4 to 7-membered heteroaryl ring and the 4 to 7-membered heterocyclic ring are substituted with 1 to 4 R⁶;

R⁶ for each occurrence is independently selected from H, C₁₋₁₀alkyl, —(CH₂CH₂O)ₙ—R^{c}, halogen, —NH(C═NH)NH₂, —OR, —NR′R″, —NO₂, —NCO, —NR′COR″, —SR, —SOR′, —SO₂R′, —SO₃H, —OSO₃H, —SO₂NR′R″, —CN, —N₃, —COR′, —OCOR′, and —OCONR′R″; and

R^{L} is a self-immolative linker comprising a reactive group that can form a covalent bond with a cell-binding agent, provided that the compound of formula (I) is not:

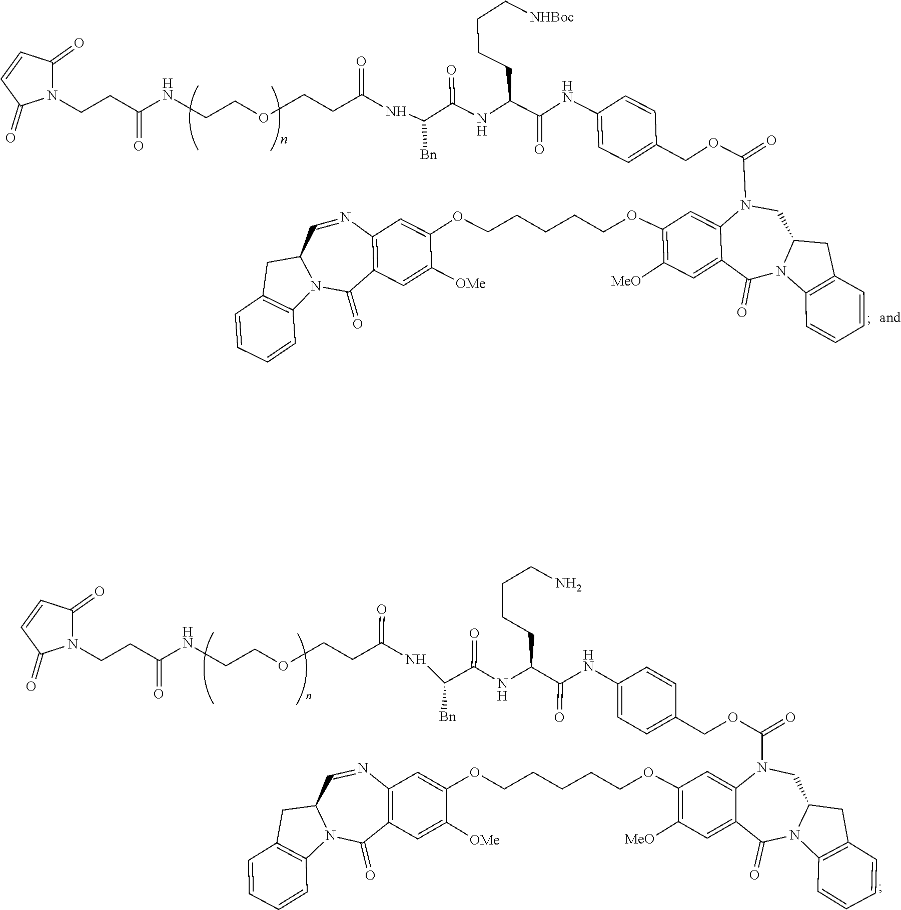

and provided the compound of formula (I^{m}) is not:

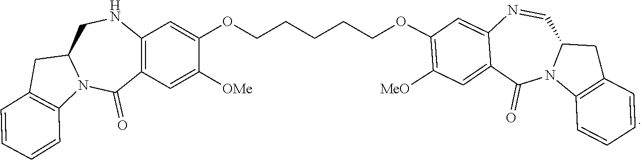 [SMILES: COc1cc2c(cc1OCCCCCOc1cc3c(cc1OC)C(=O)N1c4ccccc4C[C@H]1CN3)N=C[C@@H]1Cc3ccccc3N1C2=O]

In a second aspect, the present invention is directed to a cell-binding agent-cytotoxic agent conjugate represented by the following formula:

CBA-(-Cy)_{w}

or a pharmaceutically acceptable salt thereof, wherein:

CBA is a cell-binding agent;

Cy is a cytotoxic agent represented by the following formula:

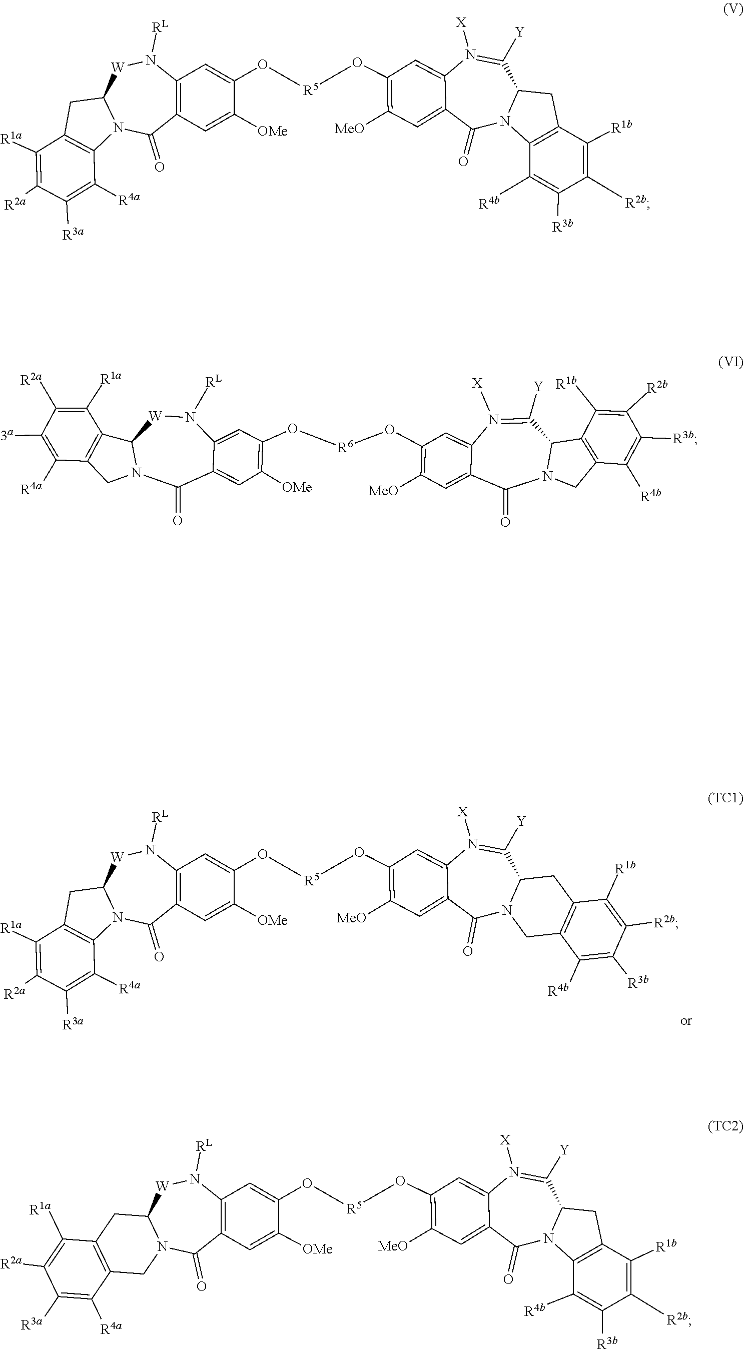

or a pharmaceutically acceptable salt thereof, wherein:

the double line [symbol] between N and C represents a single bond or a double bond, provided that when it is a double bond X is absent and Y is H, or a C₁₋₄alkyl, and when it is a single bond, X is H and Y is —OH or —SO₃H;

W is —C(═O)— or —C(Y′)—;

Y′ is H or C₁₋₄alkyl;

R^{1a}, R^{2a}, R^{3a}, R^{4a}, R^{1b}, R^{2b}, R^{3b} and R^{4b} are each independently selected from the group consisting of H, a C₁₋₁₀alkyl, —(OCH₂CH₂)ₙ—OR^{c}, halogen, —NH(C═NH)NH₂, —OR, —NR′R″, —NO₂, —NR′COR″, —SR, —SOR′, —SO₂R′, —SO₃H, —OSO₃H, —SO₂NR′R″, —CN, —N₃, —COR′, —OCOR′, and —OCONR′R″;

R^{c} is H or a C₁₋₄alkyl;

n is an integer from 1 to 24;

R, for each occurrence, is independently selected from the group consisting of H, —(CH₂CH₂O)ₙ—R^{c}, C₁₋₁₀alkyl, a C₃₋₈cycloalkyl, a 6- to 18-membered aryl, a 5- to 18-membered heteroaryl ring containing one or more heteroatoms independently selected from N, O and S, or a 3- to 18-membered heterocyclic ring containing 1 to 6 heteroatoms independently selected from O, S, N and P;

R′ and R″ are each independently selected from —H, —OH, —OR, —NHR, —NR₂, —COR, a C₁₋₁₀alkyl, a-(CH₂CH₂O)ₙ—R^{c}, and a 3- to 18-membered heterocyclic ring having 1 to 6 heteroatoms independently selected from O, S, N and P;

R⁵ is a C₃₋₁₂alkylene, which chain can be interrupted by one or more groups selected from —O—, —S—, —NH—, —NMe-, benzene ring, a 4 to 7-membered heteroaryl ring and a 4 to 7-membered heterocyclic ring, wherein the benzene, the 4 to 7-membered heteroaryl ring and the 4 to 7-membered heterocyclic ring are substituted with 1 to 4 R⁶;

R⁶ for each occurrence is independently selected from H, C₁₋₁₀alkyl, —(CH₂CH₂O)ₙ—R^{c}, halogen, —NH(C═NH)NH₂, —OR, —NR′R″, —NO₂, —NCO, —NR′COR″, —SR, —SOR′, —SO₂R′, —SO₃H, —OSO₃H, —SO₂NR′R″, —CN, —N₃, —COR′, —OCOR′, and —OCONR′R″; and

R^{L1} is a self-immolative linker covalently linked to the CBA, provided the conjugate of formula (V) is not:

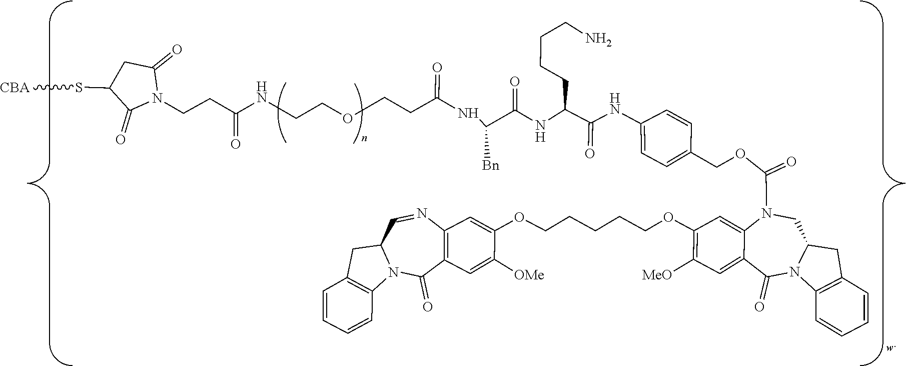

The present invention also includes a composition (e.g., a pharmaceutical composition) comprising a cytotoxic compound or a conjugate of the present invention described herein, and a carrier (a pharmaceutically acceptable carrier). The present compounds, conjugates or compositions are useful for inhibiting abnormal cell growth or treating a proliferative disorder (e.g., cancer), an autoimmune disorder, destructive bone disorder, infectious disease, viral disease, fibrotic disease, neurodegenerative disorder, pancreatitis or kidney disease in a mammal (e.g., human).

Also included in the present invention is the use of a cytotoxic compound, a conjugate, or a composition of the present invention for the manufacture of a medicament for inhibiting abnormal cell growth or treating a proliferative disorder (e.g., cancer), an autoimmune disorder, destructive bone disorder, infectious disease, viral disease, fibrotic disease, neurodegenerative disorder, pancreatitis or kidney disease in a mammal (e.g., human).

### BRIEF DESCRIPTION OF THE FIGURES

FIG. 1 shows anti-tumor activity of anti-FRα-55 conjugate in SCID mice bearing OV90 xenografts.

FIG. 2 shows anti-tumor Activity of anti-FRα-18 conjugate in SCID mice bearing NCI-H2110 xenografts.

FIG. 3 shows anti-tumor activity of anti-EGFR-79 conjugate in SCID mice bearing FaDu xenografts.

FIG. 4 shows anti-tumor activity of anti-FRα-46 conjugate in SCID mice bearing Ishikawa xenografts.

FIG. 5 shows anti-tumor activity of anti-FRα-18 conjugate in SCID mice bearing KB xenografts.

FIG. 6 shows anti-tumor activity of anti-FRα-46 conjugate in SCID mice bearing KB xenografts.

FIG. 7 shows anti-tumor activity of huCD19-55 conjugate in SCID mice bearing OCI-Ly18 xenografts.

FIG. 8 shows DNA binding affinities of exemplary compounds of the invention.

FIG. 9 is Mass Spectrometry (MS) data of an antibody conjugate of comparator compound A, showing a large amount of DO (unconjugated antibody) species present.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents that can be included within the scope of the present invention as defined by the claims. One skilled in the art will recognize many methods and materials similar or equivalent to those described herein, which could be used in the practice of the present invention.

It should be understood that any of the embodiments described herein, including those described under different aspects of the invention and different parts of the specification (including embodiments described only in the Examples) can be combined with one or more other embodiments of the invention, unless explicitly disclaimed or improper. Combination of embodiments are not limited to those specific combinations claimed via the multiple dependent claims.

### Definitions

The term “alkyl” or “linear or branched alkyl” as used herein refers to a saturated linear or branched monovalent hydrocarbon radical. In preferred embodiments, a straight chain or branched chain alkyl has thirty or fewer carbon atoms (e.g., C₁-C₃₀ for straight chain alkyl group and C₃-C₃₀ for branched alkyl), and more preferably twenty or fewer carbon atoms. Even more preferably, the straight chain or branched chain alkyl has ten or fewer carbon atoms (i.e., C₁-C₁₀ for straight chain alkyl group and C₃-C₁₀ for branched alkyl). In other embodiments, the straight chain or branched chain alkyl has six or fewer carbon atoms (i.e., C₁-C₆ for straight chain alkyl group or C₃-C₆ for branched chain alkyl). Examples of alkyl include, but are not limited to, methyl, ethyl, 1-propyl, 2-propyl, 1-butyl, 2-methyl-1-propyl, —CH₂CH(CH₃)₂), 2-butyl, 2-methyl-2-propyl, 1-pentyl, 2-pentyl 3-pentyl, 2-methyl-2-butyl, 3-methyl-2-butyl, 3-methyl-1-butyl, 2-methyl-1-butyl, 1-hexyl), 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. Moreover, the term “alkyl” as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. As used herein, (Cₓ-Cₓₓ)alkyl or Cₓ₋ₓₓalkyl means a linear or branched alkyl having x-xx carbon atoms.

The term “alkylene” as used herein refers to a saturated linear or branched divalent hydrocarbon radical. In preferred embodiments, a straight chain or branched chain alkylene has thirty or fewer carbon atoms (e.g., C₁-C₃₀ for straight chain alkylene group and C₃-C₃₀ for branched alkylene), and more preferably twenty or fewer carbon atoms. Even more preferably, the straight chain or branched chain alkylene has ten or fewer carbon atoms (i.e., C₁-C₁₀ for straight chain alkylene group and C₃-C₁₀ for branched alkylene). In other embodiments, the straight chain or branched chain alkylene has six or fewer carbon atoms (i.e., C₁-C₆ for straight chain alkylene group or C₃-C₆ for branched chain alkylene). As used herein, (Cₓ-Cₓₓ)alkylene or Cₓ₋ₓₓalkylene means a linear or branched alkylene having x-xx carbon atoms.

The term “alkenyl” or “linear or branched alkenyl” refers to linear or branched-chain monovalent hydrocarbon radical of two to twenty carbon atoms with at least one site of unsaturation, i.e., a carbon-carbon double bond, wherein the alkenyl radical includes radicals having “cis” and “trans” orientations, or alternatively, “E” and “Z” orientations. Examples include, but are not limited to, ethylenyl or vinyl (—CH═CH₂), allyl (—CH₂CH═CH₂), and the like. Preferably, the alkenyl has two to ten carbon atoms. More preferably, the alkyl has two to four carbon atoms.

The term “alkynyl” or “linear or branched alkynyl” refers to a linear or branched monovalent hydrocarbon radical of two to twenty carbon atoms with at least one site of unsaturation, i.e., a carbon-carbon, triple bond. Examples include, but are not limited to, ethynyl, propynyl, 1-butynyl, 2-butynyl, 1-pentynyl, 2-pentynyl, 3-pentynyl, hexynyl, and the like. Preferably, the alkynyl has two to ten carbon atoms. More preferably, the alkynyl has two to four carbon atoms.

The terms “cyclic alkyl” and “cycloalkyl” can be used interchangeably. As used herein, the term refers to the radical of a saturated carbocyclic ring. In preferred embodiments, cycloalkyls have from 3 to 10 carbon atoms in their ring structure, and more preferably from 5 to 7 carbon atoms in the ring structure. In some embodiments, the two cyclic rings can have two or more atoms in common, e.g., the rings are “fused rings.” Suitable cycloalkyls include, but are not limited to cycloheptyl, cyclohexyl, cyclopentyl, cyclobutyl and cyclopropyl. In some embodiments, the cycloalkyl is a monocyclic group. In some embodiments, the cycloalkyl is a bicyclic group. In some embodiments, the cycloalkyl is a tricyclic group.

The term “cycloalklalkyl” refers to an alkyl group described above that is substituted with a cycloalkyl group.

The term “cyclic alkenyl” refers to a carbocyclic ring radical having at least one double bond in the ring structure.

The term “cyclic alkynyl” refers to a carbocyclic ring radical having at least one triple bond in the ring structure.

The term “aryl” or “aromatic ring” as used herein, include substituted or unsubstituted single-ring aromatic groups in which each atom of the ring is carbon. Preferably the ring is a 5- to 7-membered ring, more preferably a 6-membered ring. Aryl groups include, but are not limited to, phenyl, phenol, aniline, and the like. The terms “aryl” also includes “polycyclyl”, “polycycle”, and “polycyclic” ring systems having two or more rings in which two or more atoms are common to two adjoining rings, e.g., the rings are “fused rings,” wherein at least one of the rings is aromatic, e.g., the other cyclic rings can be cycloalkyls, cycloalkenyls, cycloalkynyls, or aromatic rings. In some preferred embodiments, polycycles have 2-3 rings. In certain preferred embodiments, polycyclic ring systems have two cyclic rings in which both of the rings are aromatic. Each of the rings of the polycycle can be substituted or unsubstituted. In certain embodiments, each ring of the polycycle contains from 3 to 10 carbon atoms in the ring, preferably from 5 to 7. For example, aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl, and the like. In some embodiments, the aryl is a single-ring aromatic group. In some embodiments, the aryl is a two-ring aromatic group. In some embodiments, the aryl is a three-ring aromatic group.

The terms “heterocycle,” “heterocyclyl,” and “heterocyclic ring” as used herein, refers to substituted or unsubstituted non-aromatic ring structures of 3- to 18-membered rings, preferably 3- to 10-membered rings, more preferably 3- to 7-membered rings, whose ring structures include at least one heteroatom, preferably one to four heteroatoms, more preferably one or two heteroatoms. In certain embodiments, the ring structure can have two cyclic rings. In some embodiments, the two cyclic rings can have two or more atoms in common, e.g., the rings are “fused rings.” Heterocyclyl groups include, for example, piperidine, piperazine, pyrrolidine, morpholine, lactones, lactams, and the like. Heterocycles are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566. Examples of heterocyclic rings include, but are not limited to, tetrahydrofurane, dihydrofuran, tetrahydrothiene, tetrahydropyran, dihydropyran, tetrahydrothiopyran, thiomorpholine, thioxane, homopiperazine, azetidine, oxetane, thietane, homopiperidine, piperidine, piperazine, pyrrolidine, morpholine, oxepane, thiepane, oxazepine, diazepine, thiazepine, 2-pyrroline, 3-pyrroline, indoline, 2H-pyrane, 4H-pyrane, dioxane, 1,3-dioxolane, pyrazoline, dithiane, dithiolane, dihydropyrane, dihydrothiene, dihydrofurane, pyrazolidinylimidazoline, imidazolidine, 3-azabicyco[3.1.0]hexane, 3-azabicyclo[4.1.0]heptane, and azabicyclo[2.2.2]hexane. Spiro moieties are also included within the scope of this definition. Examples of a heterocyclic group wherein ring atoms are substituted with oxo (═O) moieties are pyrimidinone and 1,1-dioxo-thiomorpholine.

The term “heteroaryl” or “heteroaromatic ring” as used herein, refers to substituted or unsubstituted aromatic single ring structures, preferably 6- to 18-membered rings, preferably 5- to 7-membered rings, more preferably 5- to 6-membered rings, whose ring structures include at least one heteroatom (e.g., O, N, or S), preferably one to four or one to three heteroatoms, more preferably one or two heteroatoms. When two or more heteroatoms are present in a heteroaryl ring, they may be the same or different. The term “heteroaryl” also includes “polycyclyl”, “polycycle”, and “polycyclic” ring systems having two or more cyclic rings in which two or more ring atoms are common to two adjoining rings, e.g., the rings are “fused rings,” wherein at least one of the rings is heteroaromatic, e.g., the other cyclic rings can be cycloalkyls, cycloalkenyls, cycloalkynyls, aryls, heteroaromatics, and/or heterocyclyls. In some preferred embodiments, polycyclic heteroaryls have 2-3 rings. In certain embodiments, preferred polycyclic heteroaryls have two cyclic rings in which both of the rings are aromatic. In certain embodiments, each ring of the polycycle contains from 3 to 10 atoms in the ring, preferably from 5 to 7 atoms in the ring. For examples, heteroaryl groups include, but are not limited to, pyrrole, furan, thiophene, imidazole, oxazole, thiazole, pyrazole, pyridine, pyrazine, pyridazine, quinoline, pyrimidine, indolizine, indole, indazole, benzimidazole, benzothiazole, benzofuran, benzothiophene, cinnoline, phthalazine, quinazoline, carbazole, phenoxazine, quinoline, purine and the like. In some embodiments, the heteroaryl is a single-ring aromatic group. In some embodiments, the heteroaryl is a two-ring aromatic group. In some embodiments, the heteroaryl is a three-ring aromatic group.

The heterocycle or heteroaryl groups can be carbon (carbon-linked) or nitrogen (nitrogen-linked) attached where such is possible. By way of example and not limitation, carbon bonded heterocycles or heteroaryls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline.

By way of example and not limitation, nitrogen bonded heterocycles or heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or O-carboline.

The heteroatoms present in heteroaryl or heterocyclyl include the oxidized forms such as NO, SO, and SO₂.

In some embodiments, the heteroaromatic ring is a 5- to 18-membered ring.

The term “halo” or “halogen” refers to fluorine (F), chlorine (Cl), bromine (Br) or iodine (I). In some embodiments, the halogen is fluorine. In some embodiments, the halogen is chlorine. In some embodiments, the halogen is bromine. In some embodiments, the halogen is iodine. As used herein, the term “haloalkyl” refers to an alkyl, as defined herein, that is substituted by one or more halo groups as defined herein. The haloalkyl can be monohaloalkyl, dihaloalkyl or polyhaloalkyl. A monohaloalkyl can have one fluoro, chloro, bromo, or iodo substituent. Dihaloalkyl or polyhaloalkyl can be substituted with two or more of the same halo atoms or a combination of different halo groups. Examples of haloalkyl include, but are not limited to, flouromethyl, difluoromethyl, trifluoromethyl, chloroamethyl, dichloromethyl, trichloromethyl, pentafluoroethyl, heptafluoropropyl, diflurochloromethyl, dichlorofluoromethyl, difluoroehthyl, diflosoropropyl, dichloroethyl and dichloropropyl.

The term “alkoxy” used herein refers to alkyl-O—, wherein alkyl is defined herein above. Examples of alkoxy include, not are not limited to, methoxy, ethoxy, propoxy, 2-propoxy, butoxy, tert-butoxy, pentyloxy, hexyloxy, and the like.

The alkyl, haloalkyl, alkoxy, alkenyl, alkynyl, cyclic alkyl, cyclic alkenyl, cyclic alkynyl, carbocyclyl, aryl, heterocyclyl and heteroaryl described above can be optionally substituted with one or more (e.g., 2, 3, 4, 5, 6 or more) substituents.

Unless specifically stated as “unsubstituted,” references to chemical moieties herein are understood to also include substituted variants. For example, reference to an “alkyl” group or moiety implicitly includes both substituted and unsubstituted variants. Examples of substituents on chemical moieties includes but is not limited to, halogen, hydroxyl, carbonyl (such as carboxyl, alkoxycarbonyl, formyl, or acyl), thiocarbonyl (such as thioester, thioacetate, or thioformate), alkoxyl, alkylthio, acyloxy, phosphoryl, phosphate, phosphonate, amino, amido, amidine, imine, cyano, nitro, azido, sulfhydryl, alkylthio, sulfate, sulfonate, sulfamoyl, sulfonamido, sulfonyl, heterocyclyl, aralkyl, or aryl or heteroaryl moiety.

“Optional” or “optionally” means that the subsequently described circumstance may or may not occur, so that the application includes instances where the circumstance occurs and instances where it does not. For example, the phrase “optionally substituted” means that a nonhydrogen substituent may or may not be present on a given atom, and, thus, the application includes structures wherein a non-hydrogen substituent is present and structures wherein a nonhydrogen substituent is not present.

The term “substituted” refers to moieties having substituents replacing a hydrogen on one or more carbons, nitrogens, oxygens or sulfurs atoms. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of the invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a carbon of a substituent is described as being optionally substituted with one or more of a list of substituents, one or more of the hydrogens on the carbon (to the extent there are any) can separately and/or together be replaced with an independently selected optional substituent. If a nitrogen of a substituent is described as being optionally substituted with one or more of a list of substituents, one or more of the hydrogens on the nitrogen (to the extent there are any) can each be replaced with an independently selected optional substituent. One exemplary substituent can be depicted as —NR′R″, wherein R′ and R″ together with the nitrogen atom to which they are attached, can form a heterocyclic ring. The heterocyclic ring formed from R′ and R″ together with the nitrogen atom to which they are attached can be partially or fully saturated. In some embodiments, the heterocyclic ring consists of 3 to 7 atoms. In other embodiments, the heterocyclic ring is selected from pyrrolyl, imidazolyl, pyrazolyl, triazolyl, tetrazolyl, isoxazolyl, pyridyl and thiazolyl.

This specification uses the terms “substituent,” “radical,” and “group” interchangeably.

If a group of substituents are collectively described as being optionally substituted by one or more of a list of substituents, the group can include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent can be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. Such substituents, in non-limiting examples, can be selected from a linear, branched or cyclic alkyl, alkenyl or alkynyl having from 1 to 10 carbon atoms, aryl, heteroaryl, heterocyclyl, halogen, guanidinium [—NH(C═NH)NH₂], —OR¹⁰⁰, NR¹⁰¹R¹⁰², —NO₂, —NR¹⁰¹COR¹⁰², —SR¹⁰⁰, a sulfoxide represented by —SOR¹⁰¹, a sulfone represented by —SO₂R¹⁰¹, a sulfonate —SO₃M, a sulfate —OSO₃M, a sulfonamide represented by —SO₂NR¹⁰¹R¹⁰², cyano, an azido, —COR¹⁰¹, —OCOR¹⁰¹, —OCONR¹⁰¹R¹⁰² and a polyethylene glycol unit (—OCH₂CH₂)ₙR¹⁰¹ wherein M is H or a cation (such as Na⁺ or K⁺); R¹⁰¹, R¹⁰² and R¹⁰³ are each independently selected from H, linear, branched or cyclic alkyl, alkenyl or alkynyl having from 1 to 10 carbon atoms, a polyethylene glycol unit (—OCH₂CH₂)ₙ—R¹⁰⁴, wherein n is an integer from 1 to 24, an aryl having from 6 to 10 carbon atoms, a heterocyclic ring having from 3 to 10 carbon atoms and a heteroaryl having 5 to 10 carbon atoms; and R¹⁰⁴ is H or a linear or branched alkyl having 1 to 4 carbon atoms, wherein the alkyl, alkenyl, alkynyl, aryl, heteroaryl and heterocyclyl in the groups represented by R¹⁰⁰, R¹⁰¹, R¹⁰², R¹⁰³ and R¹⁰⁴ are optionally substituted with one or more (e.g., 2, 3, 4, 5, 6 or more) substituents independently selected from halogen, —OH, —CN, —NO₂ and unsubstituted linear or branched alkyl having 1 to 4 carbon atoms. Preferably, the substituents for the optionally substituted alkyl, alkenyl, alkynyl, cyclic alkyl, cyclic alkenyl, cyclic alkynyl, carbocyclyl, aryl, heterocyclyl and heteroaryl described above include halogen, —CN, —NR¹⁰²R¹⁰³, —CF₃, —OR¹⁰¹, aryl, heteroaryl, heterocyclyl, —SR¹⁰¹, —SOR¹⁰¹, —SO₂R¹⁰¹ and —SO₃M.

The number of carbon atoms in a group can be specified herein by the prefix “Cₓ₋ₓₓ” or “Cₓ-Cₓₓ”, wherein x and xx are integers. For example, “C₁₋₄alkyl” or “C1-C4 alkyl” is an alkyl group having from 1 to 4 carbon atoms.

The term “compound” or “cytotoxic compound,” “cytotoxic dimer” and “cytotoxic dimer compound” are used interchangeably. They are intended to include compounds for which a structure or formula or any derivative thereof has been disclosed in the present invention or a structure or formula or any derivative thereof that has been incorporated by reference. The term also includes, stereoisomers, geometric isomers, tautomers, solvates, metabolites, salts (e.g., pharmaceutically acceptable salts) and prodrugs, and prodrug salts of a compound of all the formulae disclosed in the present invention. The term also includes any solvates, hydrates, and polymorphs of any of the foregoing. The specific recitation of “stereoisomers,” “geometric isomers,” “tautomers,” “solvates,” “metabolites,” “salt” “prodrug,” “prodrug salt,” “conjugates,” “conjugates salt,” “solvate,” “hydrate,” or “polymorph” in certain aspects of the invention described in this application shall not be interpreted as an intended omission of these forms in other aspects of the invention where the term “compound” is used without recitation of these other forms.

The term “conjugate” as used herein refers to a compound described herein or a derivative thereof that is linked to a cell binding agent.

The term “chiral” refers to molecules that have the property of non-superimposability of the mirror image partner, while the term “achiral” refers to molecules that are superimposable on their mirror image partner.

The term “stereoisomer” refers to compounds that have identical chemical constitution and connectivity, but different orientations of their atoms in space that cannot be interconverted by rotation about single bonds.

The term “diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as crystallization, electrophoresis and chromatography.

The term “enantiomers” refer to two stereoisomers of a compound that are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds,” John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and I or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereo selection or stereo specificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

The term “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt can involve the inclusion of another molecule such as an acetate ion, a succinate ion or other counter ion. The counter ion can be any organic or inorganic moiety that stabilizes the charge on the parent compound. Furthermore, a pharmaceutically acceptable salt can have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt can be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt can be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

As used herein, the term “solvate” means a compound that further includes a stoichiometric or non-stoichiometric amount of solvent such as water, isopropanol, acetone, ethanol, methanol, DMSO, ethyl acetate, acetic acid, and ethanolamine dichloromethane, 2-propanol, or the like, bound by non-covalent intermolecular forces. Solvates or hydrates of the compounds are readily prepared by addition of at least one molar equivalent of a hydroxylic solvent such as methanol, ethanol, 1-propanol, 2-propanol or water to the compound to result in solvation or hydration of the imine moiety.

The phrase “pharmaceutically acceptable” indicates that the substance or composition must be compatible chemically and/or toxicologically, with the other ingredients comprising a formulation, and/or the mammal being treated therewith.

The term “leaving group” refers to an group of charged or uncharged moiety that departs during a substitution or displacement. Such leaving groups are well known in the art and include, but not limited to, halogens, esters, alkoxy, hydroxyl, tosylates, triflates, mesylates, nitriles, azide, carbamate, disulfides, thioesters, thioethers and diazonium compounds.

The term “reactive ester” refers to an ester having an easily displaceable leaving group that can readily react with an amine group to form an amide bond. Examples of reactive esters include, but are not limited to, N-hydroxysuccinimide ester, N-hydroxy sulfosuccinimide ester, nitrophenyl (e.g., 2 or 4-nitrophenyl) ester, dinitrophenyl (e.g., 2,4-dinitrophenyl) ester, sulfo-tetraflurophenyl (e.g., 4 sulfo-2,3,5,6-tetrafluorophenyl) ester, or pentafluorophenyl ester.

The term “reactive group” refers to a group that can react with a moiety located on another molecule, such as the cell-binding agent or the cytotoxic compound, to form a covalent bond. The reactive group includes, but is not limited an amine reactive group and a thiol reactive group.

The term “amine reactive group” refers to a group that can react with an amine group to form a covalent bond. Exemplary amine reactive groups include, but are not limited to, reactive ester groups, acyl halides, sulfonyl halide, imidoester, or a reactive thioester groups. In certain embodiments, the amine reactive group is a reactive ester group. In one embodiment, the amine reactive group is a N-hydroxysuccinimide ester or a N-hydroxy sulfo-succinimide ester.

The term “thiol-reactive group” refers to a group that can react with a thiol (—SH) group to form a covalent bond. Exemplary thiol-reactive groups include, but are not limited to, maleimide, haloacetyl, aloacetamide, vinyl sulfone, vinyl sulfonamide or vinyal pyridine. In one embodiment, the thiol-reactive group is maleimide.

The term “bifunctional crosslinking agent,” “bifunctional linker” or “crosslinking agents” refers to modifying agents that possess two reactive groups; one of which is capable of reacting with a cell-binding agent while the other one reacts with the cytotoxic compound to link the two moieties together. Such bifunctional crosslinkers are well known in the art (see, for example, Isalm and Dent in Bioconjugation chapter 5, p218-363, Groves Dictionaries Inc. New York, 1999). For example, bifunctional crosslinking agents that enable linkage via a thioether bond include N-succinimidyl-4-(N-maleimidomethyl)-cyclohexane-1-carboxylate (SMCC) to introduce maleimido groups, or with N-succinimidyl-4-(iodoacetyl)-aminobenzoate (SIAB) to introduce iodoacetyl groups. Other bifunctional crosslinking agents that introduce maleimido groups or haloacetyl groups on to a cell binding agent are well known in the art (see US Patent Applications 2008/0050310, 20050169933, available from Pierce Biotechnology Inc. P.O. Box 117, Rockland, Ill. 61105, USA) and include, but not limited to, bis-maleimidopolyethyleneglycol (BMPEO), BM(PEO)₂, BM(PEO)₃, N-(β-maleimidopropyloxy)succinimide ester (BMPS), γ-maleimidobutyric acid N-succinimidyl ester (GMBS), ε-maleimidocaproic acid N-hydroxysuccinimide ester (EMCS), 5-maleimidovaleric acid NHS, HBVS, N-succinimidyl-4-(N-maleimidomethyl)-cyclohexane-1-carboxy-(6-amidocaproate), which is a “long chain” analog of SMCC (LC-SMCC), m-maleimidobenzoyl-N-hydroxysuccinimide ester (MBS), 4-(4-N-maleimidophenyl)-butyric acid hydrazide or HCl salt (MPBH), N-succinimidyl 3-(bromoacetamido)propionate (SBAP), N-succinimidyl iodoacetate (SIA), κ-maleimidoundecanoic acid N-succinimidyl ester (KMUA), N-succinimidyl 4-(p-maleimidophenyl)-butyrate (SMPB), succinimidyl-6-(β-maleimidopropionamido)hexanoate (SMPH), succinimidyl-(4-vinylsulfonyl)benzoate (SVSB), dithiobis-maleimidoethane (DTME), 1,4-bis-maleimidobutane (BMB), 1,4 bismaleimidyl-2,3-dihydroxybutane (BMDB), bis-maleimidohexane (BMH), bis-maleimidoethane (BMOE), sulfosuccinimidyl 4-(N-maleimido-methyl)cyclohexane-1-carboxylate (sulfo-SMCC), sulfosuccinimidyl(4-iodo-acetyl)aminobenzoate (sulfo-SIAB), m-maleimidobenzoyl-N-hydroxysulfo succinimide ester (sulfo-MBS), N-(γ-maleimidobutryloxy)sulfosuccinimide ester (sulfo-GMBS), N-(ε-maleimidocaproyloxy)sulfo succimido ester (sulfo-EMCS), N-(κ-maleimidoundecanoyloxy)sulfosuccinimide ester (sulfo-KMUS), and sulfosuccinimidyl 4-(p-maleimidophenyl)butyrate (sulfo-SMPB).

Heterobifunctional crosslinking agents are bifunctional crosslinking agents having two different reactive groups. Heterobifunctional crosslinking agents containing both an amine-reactive N-hydroxysuccinimide group (NHS group) and a carbonyl-reactive hydrazine group can also be used to link the cytotoxic compounds described herein with a cell-binding agent (e.g., antibody). Examples of such commercially available heterobifunctional crosslinking agents include succinimidyl 6-hydrazinonicotinamide acetone hydrazone (SANH), succinimidyl 4-hydrazidoterephthalate hydrochloride (SHTH) and succinimidyl hydrazinium nicotinate hydrochloride (SHNH). Conjugates bearing an acid-labile linkage can also be prepared using a hydrazine-bearing benzodiazepine derivative of the present invention. Examples of bifunctional crosslinking agents that can be used include succinimidyl-p-formyl benzoate (SFB) and succinimidyl-p-formylphenoxyacetate (SFPA).

Bifunctional crosslinking agents that enable the linkage of cell binding agent with cytotoxic compounds via disulfide bonds are known in the art and include N-succinimidyl-3-(2-pyridyldithio)propionate (SPDP), N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB) to introduce dithiopyridyl groups. Other bifunctional crosslinking agents that can be used to introduce disulfide groups are known in the art and are disclosed in U.S. Pat. Nos. 6,913,748, 6,716,821 and US Patent Publications 20090274713 and 20100129314, all of which are incorporated herein by reference. Alternatively, crosslinking agents such as 2-iminothiolane, homocysteine thiolactone or S-acetylsuccinic anhydride that introduce thiol groups can also be used.

The term “linker,” “linker moiety,” or “linking group” as defined herein refers to a moiety that connects two groups, such as a cell binding agent and a cytotoxic compound, together. Typically, the linker is substantially inert under conditions for which the two groups it is connecting are linked. A bifunctional crosslinking agent can comprise two reactive groups, one at each ends of a linker moiety, such that one reactive group can be first reacted with the cytotoxic compound to provide a compound bearing the linker moiety and a second reactive group, which can then react with a cell binding agent. Alternatively, one end of the bifunctional crosslinking agent can be first reacted with the cell binding agent to provide a cell binding agent bearing a linker moiety and a second reactive group, which can then react with a cytotoxic compound. The linking moiety can contain a chemical bond that allows for the release of the cytotoxic moiety at a particular site. Suitable chemical bonds are well known in the art and include disulfide bonds, thioether bonds, acid labile bonds, photolabile bonds, peptidase labile bonds and esterase labile bonds (see for example U.S. Pat. Nos. 5,208,020; 5,475,092; 6,441,163; 6,716,821; 6,913,748; 7,276,497; 7,276,499; 7,368,565; 7,388,026 and 7,414,073). Preferred are disulfide bonds, thioether and peptidase labile bonds. Other linkers that can be used in the present invention include non-cleavable linkers, such as those described in are described in detail in U.S. publication number 20050169933, or charged linkers or hydrophilic linkers and are described in US 2009/0274713, US 2010/01293140 and WO 2009/134976, each of which is expressly incorporated herein by reference, each of which is expressly incorporated herein by reference.

The term “self-immolative linker” refers to a linker that will allow for release of the, cytotoxic compound when a remote site is activated. In certain embodiments, the linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the linker comprises p-aminobenzyloxycarbonyl (PAB). Other examples of self-immolative linkers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Linkage of a drug to the α-carbon of a glycine residue is another example of a self-immolative linker that may be useful in ADC (Kingsbury et al (1984) J. Med. Chem. 27:1447).

The term “amino acid” refers to naturally occurring amino acids or non-naturally occurring amino acid. In some embodiments, the amino acid is represented by NH₂—C(R^{aa′}R^{aa})—C(═O)OH, wherein R^{aa} and R^{aa′} are each independently H, an optionally substituted linear, branched or cyclic alkyl, alkenyl or alkynyl having 1 to 10 carbon atoms, aryl, heteroaryl or heterocyclyl or R^{aa} and the N-terminal nitrogen atom can together form a heterocyclic ring (e.g., as in proline). The term “amino acid residue” refers to the corresponding residue when one hydrogen atom is removed from the amine and/or carboxy end of the amino acid, such as —NH—C(R^{aa′}R^{aa})—C(═O)—.

The term “peptide” refers to short chains of amino acid monomers linked by peptide (amide) bonds. In some embodiments, the peptides contain 2 to 20 amino acid residues. In other embodiments, the peptides contain 2 to 10 or 2 to 8 amino acid residues. In yet other embodiments, the peptides contain 2 to 5 amino acid residues. As used herein, when a peptide is a portion of a cytotoxic agent or a linker described herein represented by a specific sequence of amino acids, the peptide can be connected to the rest of the cytotoxic agent or the linker in both directions.

The term “cation” refers to an ion with positive charge. The cation can be monovalent (e.g., Na⁺, K⁺, etc.), bi-valent (e.g., Ca²⁺, Mg²⁺, etc.) or multi-valent (e.g., Al³⁺ etc.). Preferably, the cation is monovalent.

The terms “(human) IL-3Rα,” “Interleukine-3 Receptor alpha,” or “CD123,” as used interchangeably herein, refers to any native (human) IL-3Rα or CD123, unless otherwise indicated. The CD123 protein is an interleukin 3-specific subunit of a heterodimeric cytokine receptor (IL-3 Receptor, or IL-3R). The IL-3R is comprised of a ligand specific alpha subunit, and a signal transducing common beta subunit (also known as CD131) shared by the receptors for interleukin 3 (IL3), colony stimulating factor 2 (CSF2/GM-CSF), and interleukin 5 (IL5). The binding of CD123/IL-3Rα to IL3 depends on the beta subunit. The beta subunit is activated by the ligand binding, and is required for the biological activities of IL3.

All of these above terms for CD123 can refer to either a protein or nucleic acid sequence as indicated herein. The term “CD123/IL-3Rα” encompasses “full-length,” unprocessed CD123/IL-3Rα, as well as any form of CD123/IL-3Rα that results from processing within the cell. The term also encompasses naturally occurring variants of CD123/IL-3Rα protein or nucleic acid, e.g., splice variants, allelic variants and isoforms. The CD123/IL-3Rα polypeptides and polynucleotides described herein can be isolated from a variety of sources, such as from human tissue types or from another source, or prepared by recombinant or synthetic methods. Examples of CD123/IL-3Rα sequences include, but are not limited to NCBI reference numbers NP_002174 & NM_002183 (protein and nucleic acid sequences for human CD123 variant 1), and NP_001254642 & NM_001267713 (protein and nucleic acid sequences for human CD123 variant 2).

The term “ADAM9” refers to Disintegrin and Metalloproteinase Domain-containing Protein 9, which a member of the ADAM family of molecules. It is synthesized as an inactive form which is proteolytically cleaved to generate an active enzyme. Processing at the upstream site is particularly important for activation of the proenzyme. ADAM9 is expressed in fibroblasts (Zigrino, P. et al. (2011) “The Disintegrin-Like And Cysteine-Rich Domains Of ADAM-9 Mediate Interactions Between Melanoma Cells And Fibroblasts,” J. Biol. Chem. 286:6801-6807), activated vascular smooth muscle cells (Sun, C. et al. (2010) “ADAM15 Regulates Endothelial Permeability And Neutrophil Migration Via Src/ERK1/2 Signalling,” Cardiovasc. Res. 87:348-355), monocytes (Namba, K. et al. (2001) “Involvement Of ADAM9 In Multinucleated Giant Cell Formation Of Blood Monocytes,” Cell. Immunol. 213:104-113), and activated macrophages (Oksala, N. et al. (2009) “ADAM-9, ADAM-15, And ADAM-17 Are Upregulated In Macrophages In Advanced Human Atherosclerotic Plaques In Aorta And Carotid And Femoral Arteries—Tampere Vascular Study,” Ann. Med. 41:279-290). A representative human ADAM9 polypeptide is NCBI Sequence NP_003807. Of the 819 amino acid residues of the ADAM9 polypeptide, residues 1-28 are a signal sequence, residues 29-697 are the Extracellular Domain, residues 698-718 are the Transmembrane Domain, and residues 719-819 are the Intracellular Domain. Three structural domains are located within the Extracellular Domain: a Reprolysin (M12B) Family Zinc Metalloprotease Domain (at approximately residues 212-406); a Disintegrin Domain (at approximately residues 423-497); and an EGF-like Domain (at approximately residues 644-697). A number of post-translational modifications and isoforms have been identified and the protein is proteolytically cleaved in the trans-Golgi network before it reaches the plasma membrane to generate a mature protein. The removal of the pro-domain occurs via cleavage at two different sites. Processing is most likely by a pro-protein convertase such as furin, at the boundary between the pro-domain and the catalytic domain (Arg-205/Ala-206). An additional upstream cleavage pro-protein convertase site (Arg-56/Glu-57) has an important role in the activation of ADAM9. A representative cynomolgus monkey ADAM9 polypeptide is NCBI Sequence XM_005563126.2, including a possible 28 amino acid residue signal sequence. The Reprolysin (M12B) Family Zinc Metalloprotease Domain of the protein is at approximately residues 212-406); the Disintegrin Domain of the protein is at approximately residues 423-497.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)₂, and Fv fragments), single chain Fv (scFv) mutants, multispecific antibodies such as bispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

In some embodiments, an antibody is a non-naturally occurring antibody. In some embodiments, an antibody is purified from natural components. In some embodiments, an antibody is recombinantly produced. In some embodiments, an antibody is produced by a hybridoma.

The term “anti-CD123 antibody,” “anti-IL-3Rα antibody” or “an antibody that (specifically) binds to CD123/IL-3Rα” refers to an antibody that is capable of binding CD123/IL-3Rα with sufficient affinity such that the antibody is useful as a diagnostic and/or therapeutic agent in targeting CD123/IL-3Rα. Unless otherwise specified, the extent of binding of an anti-CD123/IL-3Rα antibody to an unrelated, non-CD123/IL-3Rα protein is less than about 10% of the binding of the antibody to CD123/IL-3Rα as measured, e.g., by a radioimmunoassay (RIA). In certain embodiments, an antibody that binds to CD123/IL-3Rα has a dissociation constant (K_{d}) of ≤0.5 nM, ≤0.3 nM, ≤0.1 nM, ≤0.05 nM, or ≤0.01 nM. In one embodiment, the anti-CD123/IL-3Rα antibody does not bind the common beta chain CD131. In one embodiment, the anti-CD123/IL-3Rα antibody does not bind to the same epitope of CD123 that is bound by the known and commercially available CD123 antibodies such as 7G3 (mouse IgG₂ₐ), 6H6 (mouse IgG₁), and 9F5 (mouse IgG₁) (Sun et al., Blood 87(1): 83-92, 1996).

The sequences of anti-CD123/IL-3Rα antibodies and antigen-binding fragments thereof of the invention are provided herein.

The term “antibody fragment” refers to a portion of an intact antibody and refers to the antigenic determining variable regions of an intact antibody. Examples of antibody fragments include, but are not limited to, Fab, Fab′, F(ab′)₂, and Fᵥ fragments, linear antibodies, single chain antibodies, and multispecific antibodies formed from antibody fragments. The term “antigen-binding fragment” of an antibody includes one or more fragments of an antibody that retain the ability to specifically bind to an antigen. It has been shown that the antigen-binding function of an antibody can be performed by certain fragments of a full-length antibody. Examples of binding fragments encompassed within the term “antigen-binding fragment” of an antibody include (without limitation): (i) an Fab fragment, a monovalent fragment consisting of the V_{L}, V_{H}, C_{L}, and C_{H1} domains (e.g., an antibody digested by papain yields three fragments: two antigen-binding Fab fragments, and one Fc fragment that does not bind antigen); (ii) a F(ab′)₂ fragment, a bivalent fragment comprising two Fab fragments linked by a disulfide bridge at the hinge region (e.g., an antibody digested by pepsin yields two fragments: a bivalent antigen-binding F(ab′)₂ fragment, and a pFc′ fragment that does not bind antigen) and its related F(ab′) monovalent unit; (iii) a F_{d} fragment consisting of the V_{H} and C_{H1} domains (i.e., that portion of the heavy chain which is included in the Fab); (iv) a Fᵥ fragment consisting of the V_{L} and V_{H} domains of a single arm of an antibody, and the related disulfide linked Fᵥ; (v) a dAb (domain antibody) or sdAb (single domain antibody) fragment (Ward et al., Nature 341:544-546, 1989), which consists of a V_{H} domain; and (vi) an isolated complementarity determining region (CDR).

The term “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)₂, Fᵥ), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g., murine) antibodies that are specific immunoglobulin chains, chimeric immunoglobulins, or fragments thereof that contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, hamster) that have the desired specificity, affinity, and capability (Jones et al., Nature 321:522-525, 1986; Riechmann et al., Nature 332:323-327, 1988; Verhoeyen et al., Science 239:1534-1536, 1988).

In some instances, the Fᵥ framework region (FR) residues of a human immunoglobulin are replaced with the corresponding residues in an antibody from a non-human species that has the desired specificity, affinity, and capability. The humanized antibody can be further modified by the substitution of additional residues either in the Fᵥ framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fₑ), typically that of a human immunoglobulin. Examples of methods used to generate humanized antibodies are described in U.S. Pat. Nos. 5,225,539 and 5,639,641, Roguska et al., Proc. Natl. Acad. Sci. USA 91(3):969-973, 1994; and Roguska et al., Protein Eng. 9(10):895-904, 1996 (all incorporated herein by reference). In some embodiments, a “humanized antibody” is a resurfaced antibody. In some embodiments, a “humanized antibody” is a CDR-grafted antibody.

The term “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, 5th ed., 1991, National Institutes of Health, Bethesda Md.); and (2) an approach based on crystallographic studies of antigen-antibody complexes (Al-lazikani et al., J. Molec. Biol. 273:927-948, 1997). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., Kabat et al., Sequences of Immunological Interest, 5th Ed., Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed., Public Health Service, National Institutes of Health, Bethesda, Md. (1991) (incorporated herein by reference). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917,1987). The end of the Chothia CDR-H1 loop when numbered using the Kabat numbering convention varies between H32 and H34 depending on the length of the loop. This is because the Kabat numbering scheme places the insertions at H35A and H35B—if neither 35A nor 35B is present, the loop ends at 32; if only 35A is present, the loop ends at 33; if both 35A and 35B are present, the loop ends at 34. The AbM hypervariable regions represent a compromise between the Kabat CDRs and Chothia structural loops, and are used by Oxford Molecular's AbM antibody modeling software.

| Loop | Kabat | AbM | Chiothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H9S-H102 | H95-H102 | H95-H102 |

The EU index or EU index as in Kabat or EU numbering scheme refers to the numbering system based on the human IgG1 Eu antibody of Edelman et al., 1969, Proc Natl Acad Sci USA 63:78-85, incorporated herein by reference.

The term “human antibody” means an antibody produced by a human or an antibody having an amino acid sequence corresponding to an antibody produced by a human made using any technique known in the art. In certain embodiments, the human antibody does not have non-human sequence. This definition of a human antibody includes intact or full-length antibodies, or antigen-binding fragments thereof.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid or reduce the chance of eliciting an immune response in that species (e.g., human). In certain embodiments, chimeric antibody may include an antibody or antigen-binding fragment thereof comprising at least one human heavy and/or light chain polypeptide, such as, for example, an antibody comprising murine light chain and human heavy chain polypeptides.

The terms “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}) or the half-maximal effective concentration (EC₅₀). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described herein.

By “specifically binds,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

The term “immunoconjugate,” “conjugate,” or “ADC” as used herein refers to a compound or a derivative thereof that is linked to a cell binding agent (e.g., an antibody or antigen-binding fragment thereof).

The term “cysteine-engineered antibody” includes an antibody with at least one Cys that is not normally present at a given residue of the antibody light chain or heavy chain. Such Cys, which may also be referred to as “engineered Cys,” can be engineered using any conventional molecular biology or recombinant DNA technology (e.g., by replacing the coding sequence for a non-Cys residue at the target residue with a coding sequence for Cys). For example, if the original residue is Ser with a coding sequence of 5′-UCU-3′, the coding sequence can be mutated (e.g., by site-directed mutagenesis) to 5′-UGU-3′, which encodes Cys. In certain embodiments, the Cys engineered antibody of the invention has an engineered Cys in the heavy chain. In certain embodiments, the engineered Cys is in or near the CH3 domain of the heavy chain. In certain embodiments, the engineered Cys is at residue 442 of the heavy chain (EU/OU numbering). The C442 residue can be conjugated with a cytotoxic drug/agent through the free thiol group of the C442 residue, such as through reacting with a thiol-reactive agent of the cytotoxic drug (e.g., a maleimido group).

The terms “cancer” and “cancerous” refer to or describe the physiological condition in mammals in which a population of cells are characterized by unregulated cell growth. “Tumor” and “neoplasm” refer to one or more cells that result from excessive cell growth or proliferation, either benign (noncancerous) or malignant (cancerous) including pre-cancerous lesions.

Examples of cancer include endometrial cancer, lung cancer (e.g., non-small-cell lung cancer), colorectal cancer, bladder cancer, gastric cancer, pancreatic cancer, renal cell carcinoma, prostate cancer, esophageal cancer, breast cancer, head and neck cancer, uterine cancer, ovarian cancer, liver cancer, cervical cancer, thyroid cancer, testicular cancer, myeloid cancer, melanoma, and lymphoid cancer. In certain embodiments, the cancer is non-small-cell lung cancer, colorectal cancer, gastric cancer or pancreatic cancer. In certain embodiments, the cancer is non-small-cell lung cancer (squamous cell, nonsquamous cell, adenocarcinoma, or large-cell undifferentiated carcinoma), colorectal cancer (adenocarcinoma, gastrointestinal carcinoid tumors, gastrointestinal stromal tumors, primary colorectal lymphoma, leiomyosarcoma, or squamous cell carcinoma) or breast cancer (e.g., triple negative breast cancer (TNBC)). In certain embodiments, cancer is lymphoma and leukemia. In certain embodiments, examples of cancers include AML, CML, ALL (e.g., B-ALL), CLL, myelodysplastic syndrome, basic plasmacytoid DC neoplasm (BPDCN) leukemia, B-cell lymphomas including non-Hodgkin lymphomas (NHL), precursor B-cell lymphoblastic leukemia/lymphoma and mature B-cell neoplasms, such as B-cell chronic lymphocytic leukemia (B-CLL)/small lymphocytic lymphoma (SLL), B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia (HCL), diffuse large B-cell lymphoma (DLBCL), Burkitt's lymphoma, plasmacytoma, plasma cell myeloma, post-transplant lymphoproliferative disorder, Waldenstrom's macroglobulinemia, anaplastic large-cell lymphoma (ALCL), and Hodgkin's leukemia (HL). In certain embodiments, the cancer is BPDCN leukemia. In certain embodiments, the cancer is ALL. In other embodiments, the cancer is AML.

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

A “therapeutically effective amount” of an immunoconjugate as disclosed herein is an amount sufficient to carry out a specifically stated purpose. A “therapeutically effective amount” can be determined empirically and in a routine manner, in relation to the stated purpose.

The term “imine reactive reagent” refers to a reagent that is capable of reacting with an imine group. Preferably, the imine reactive reagent is selected from sulfites, hydroxyl amine, urea and hydrazine. More preferably, the imine reactive reagent is NaHSO₃ or KHSO₃.

### Compounds of the Present Invention

In a first aspect, the present invention is directed to cytotoxic compounds described herein. In certain embodiments, the cytotoxic compounds of the present invention is a benzodiazepine dimer compound having a self-immolative linker that can be linked to a cell-binding agent (CBA) at the N-10 amine of the reduced benzodiazepine monomer.

In a 1^{st} embodiment, the compound of the present invention is represented by the formula (I), (II), (TI), (T2), (I^{m}), (II^{m}), (T1^{m}) or (T2^{m}), or a pharmaceutically acceptable salt thereof, wherein:

the double line [symbol] between N and C represents a single bond or a double bond, provided that when it is a double bond X is absent and Y is H, or a C₁₋₄alkyl, and when it is a single bond, X is H and Y is —OH or —SO₃H;

W is —C(═O)— or —C(Y′)—;

Y′ is H or C₁₋₄alkyl;

R^{1a}, R^{2a}, R^{3a}, R^{4a}, R^{1b}, R^{2b}, R^{3b} and R^{4b} are each independently selected from the group consisting of H, a C₁₋₁₀alkyl, —(OCH₂CH₂)ₙOR^{c}, halogen, —NH(C═NH)NH₂, —OR, —NR′R″, —NO₂, —NR′COR″, —SR, —SOR′, —SO₂R′, —SO₃H, —OSO₃H, —SO₂NR′R″, —CN, —N₃, —COR′, —OCOR′, and —OCONR′R″;

R^{c} is H or a C₁₋₄alkyl;

n is an integer from 1 to 24;

R, for each occurrence, is independently selected from the group consisting of H, —(CH₂CH₂O)ₙ—R^{c}, C₁₋₁₀alkyl, a C₃₋₈cycloalkyl, a 6- to 18-membered aryl, a 5- to 18-membered heteroaryl ring containing one or more heteroatoms independently selected from N, O and S, or a 3- to 18-membered heterocyclic ring containing 1 to 6 heteroatoms independently selected from O, S, N and P;

R′ and R″ are each independently selected from —H, —OH, —OR, —NHR, —NR₂, —COR, a C₁₋₁₀alkyl, a-(CH₂CH₂O)ₙ—R^{c}, and a 3- to 18-membered heterocyclic ring having 1 to 6 heteroatoms independently selected from O, S, N and P;

R⁵ is a C₃₋₁₂alkylene, which chain can be interrupted by one or more groups selected from —O—, —S—, —NH—, —NMe-, benzene ring, a 4 to 7-membered heteroaryl ring and a 4 to 7-membered heterocyclic ring, wherein the benzene, the 4 to 7-membered heteroaryl ring and the 4 to 7-membered heterocyclic ring are substituted with 1 to 4 R⁶;

R⁶ for each occurrence is independently selected from H, C₁₋₁₀alkyl, —(CH₂CH₂O)ₙ—R^{c}, halogen, —NH(C═NH)NH₂, —OR, —NR′R″, —NO₂, —NCO, —NR′COR″, —SR, —SOR′, —SO₂R′, —SO₃H, —OSO₃H, —SO₂NR′R″, —CN, —N₃, —COR′, —OCOR′, and —OCONR′R″; and

R^{L} is a self-immolative linker comprising a reactive group that can form a covalent bond with a cell-binding agent, provided that the compound of formula (I) is not:

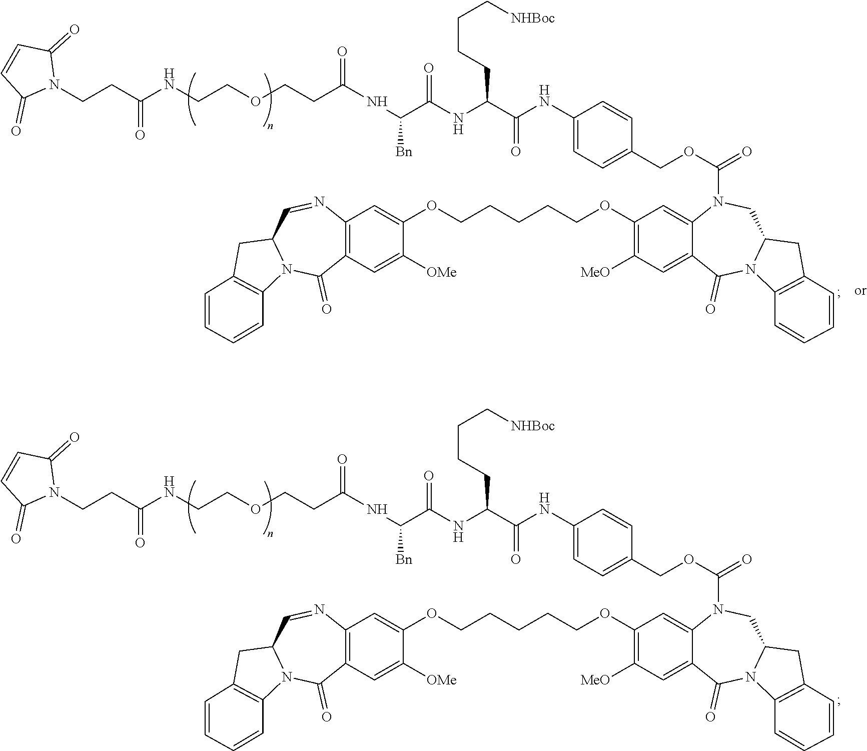

and provided the compound of formula (I^{m}) is not:

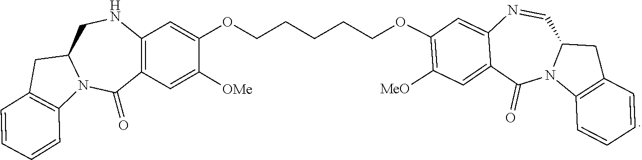 [SMILES: COc1cc2c(cc1OCCCCCOc1cc3c(cc1OC)C(=O)N1c4ccccc4C[C@H]1CN3)N=C[C@@H]1Cc3ccccc3N1C2=O]

In a 2^{nd} embodiment, the compound of the present invention is represented by one of the following formulae in Table A:

| | |
|---|---|
| 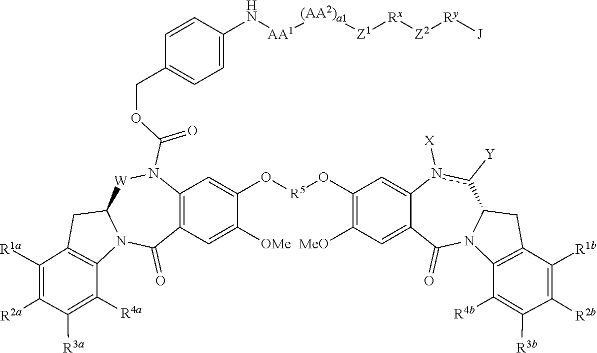 | (IIIa) |
| 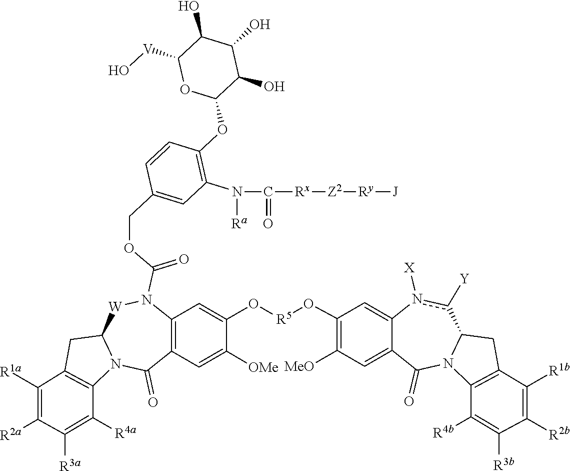 | (IIIb) |
| 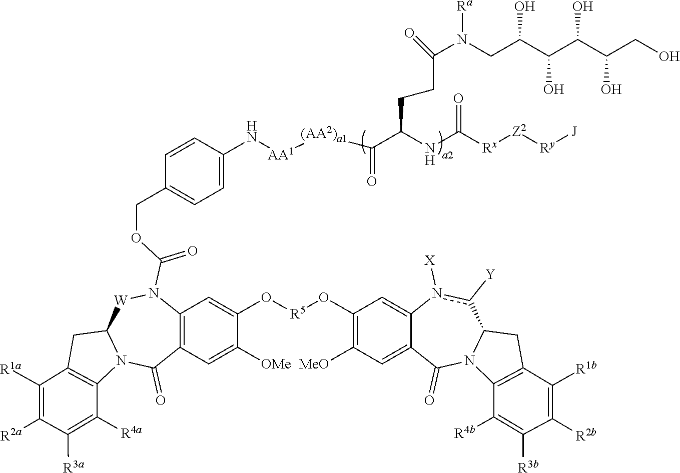 | (IIIc) |
| 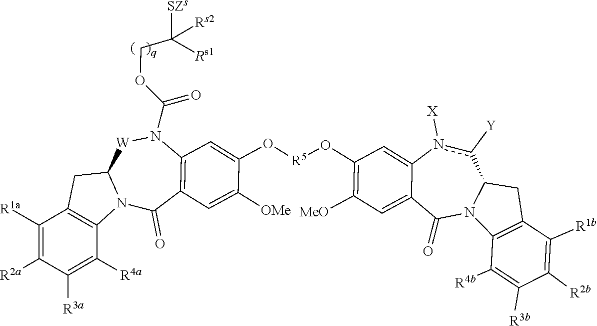 | (IIId) |
| 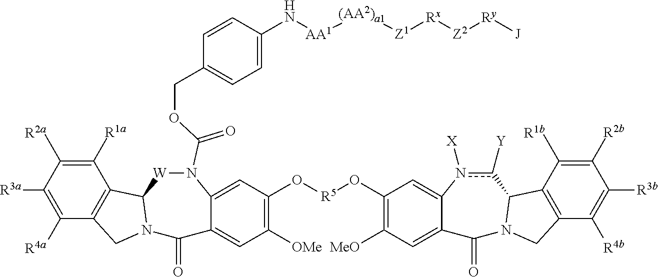 | (IVa) |
| 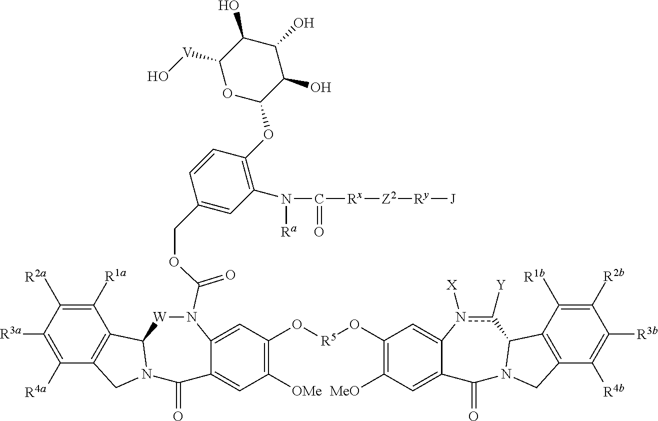 | (IVb) |
| 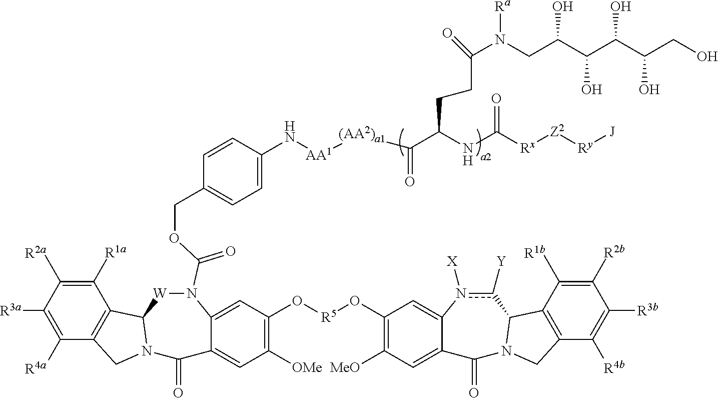 | (IVc) |
| 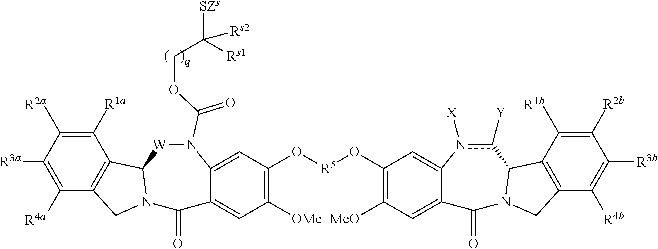 | (IVd) |
| 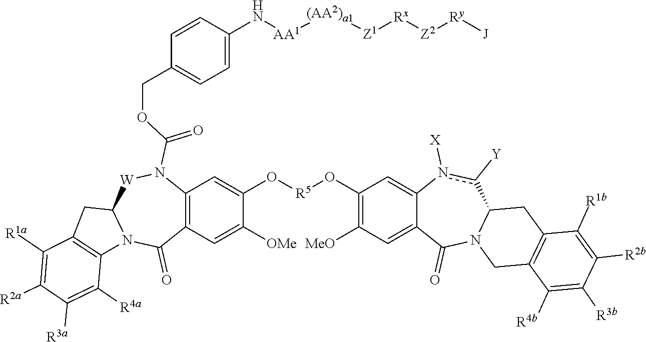 | (T1a) |
| 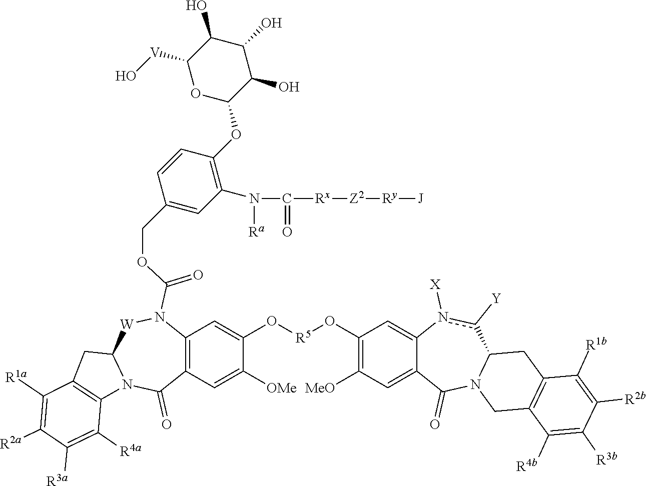 | (T1b) |
| 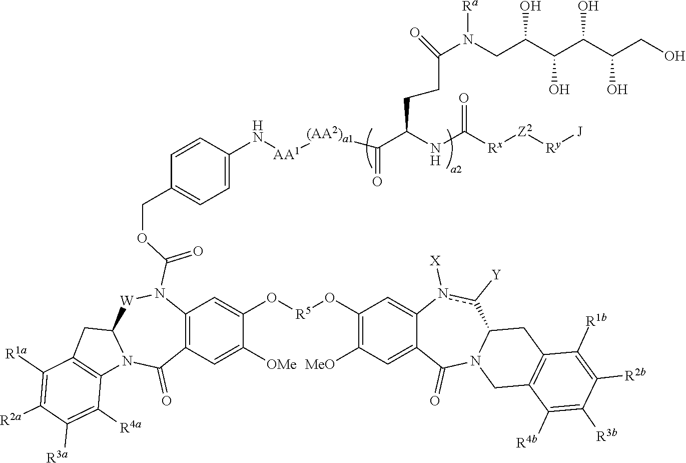 | (T1c) |
| 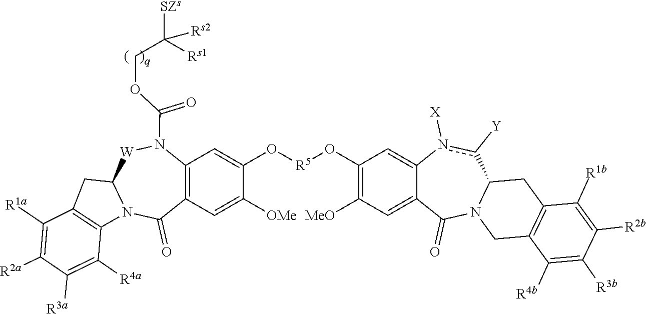 | (T1d) |
| 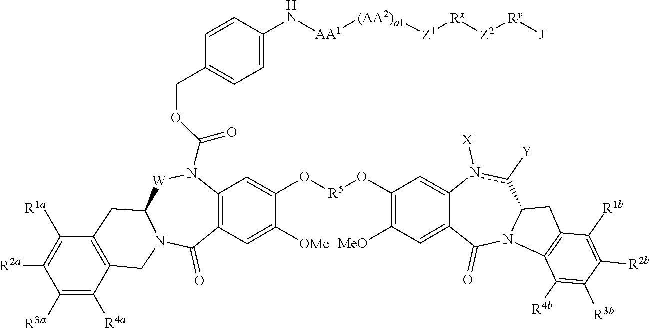 | (T2a) |
| 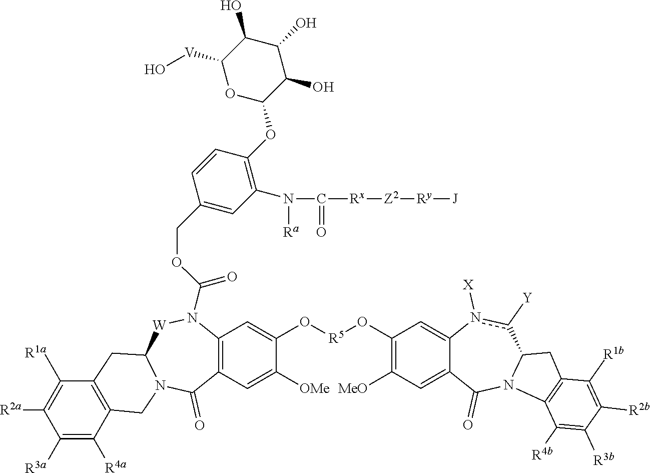 | (T2b) |
| 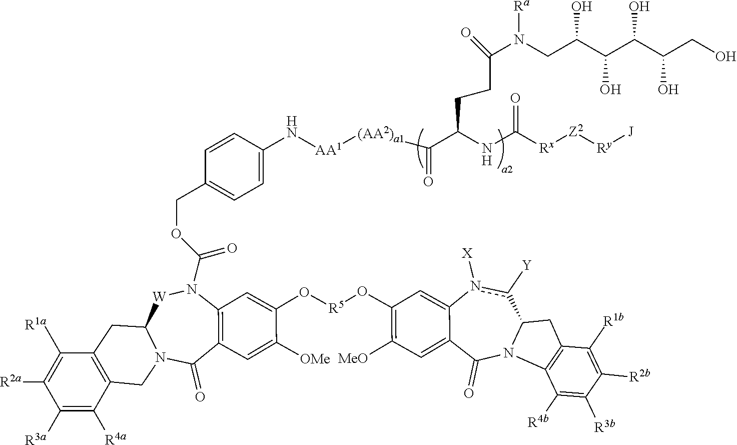 | (T2c) |
| 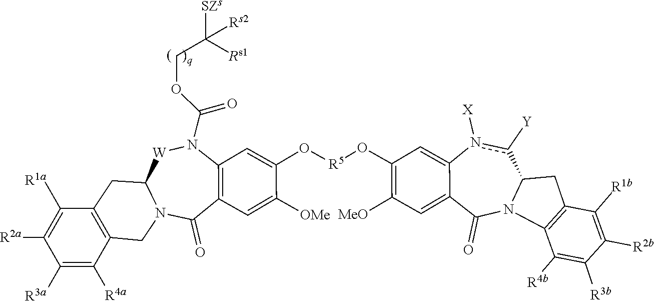 | (T2d) |

or a pharmaceutically acceptable salt thereof, wherein:

AA′ and AA² are each independently an amino acid residues;

a1 is an integer from 1 to 19;

a2 is an integer from 1 to 5;

R^{a} is H or C₁₋₄alkyl;

q is 1, 2 or 3;

R^{s1} and R^{s2} are each independently H or C₁₋₄alkyl, or R^{s1} and R^{s2} taken together with the carbon atom to which they are attached form a 3 to 5-membered cycloalkyl ring, provided when q is 1, R^{s1} and R^{s2} taken together with the carbon atom to which they are attached cannot form a 3-membered cycloalkyl ring;

V is C(═O) or CH₂

Z¹ is —C(═O)— or —SO₂—NH—C(═O)—, wherein the —SO₂— group in —SO₂—NH—C(═O)— is connected to P¹;

R^{x} is C₁₋₁₀alkylene, C₃₋₈cycloalkyl, —(CH₂CH₂O)ₘ₁—C₁₋₁₀alkylene- or C₁₋₁₀alkylene-(OCH₂CH₂)ₘ₂—;

m1 and m2 are each independently an integer from 1 to 24;

Z² is absent, —C(═O)NH— or —NH—C(═O)—;

R^{y} is absent, C₁₋₁₀alkylene, —(CH₂CH₂O)ₘ₃—C₁₋₁₀alkylene- or C₁₋₁₀alkylene-(OCH₂CH₂)ₘ₄—;

m3 and m4 are each independently an integer from 1 to 24;

Z^{s} is a bifunctional crosslinker bearing a reactive group that is covalently linked to the cytotoxic compound via a disulfide bond or a thioether bond;

J is a moiety comprising a reactive group (preferably, an amine reactive group or a thiol reactive group) that is capable of forming a covalent bond with a cell-binding agent; and the remaining variables are as defined in the 1^{st} embodiment.

In a specific embodiment, for formula (IIId), (IVd), (T1d) or (T2d), q is 1. In a more specific embodiment, for formula (IIId), (IVd), (T1d) or (T2d), q is 1; and R^{s1} and R^{s2} are both methyl.

In a specific embodiment, for formula (IIIb), (IIIc), (IVb), (IVc), (T1b), (T1c), (T2b) or (T2c), R^{a} is H, methyl or ethyl. In a more specific embodiment, R^{a} is H. In a more specific embodiment, R^{a} is methyl.

In a 3^{rd} embodiment, the compound of the present invention is represented by a formula described in the 1^{st} or 2^{nd} embodiment, or a pharmaceutically acceptable salt thereof, wherein R^{1a}, R^{2a}, R^{3a}, R^{4a}, R^{1b}, R^{2b}, R^{3b} and R^{4b} are all H; and the remaining variables are as defined in the 1^{st} or 2^{nd} embodiment or any specific embodiment described therein.

In a 4^{th} embodiment, the compound of the present invention is represented by a formula described in the 1^{st} or 2^{nd} embodiment, or a pharmaceutically acceptable salt thereof, wherein R⁵ is a C₃₋₇alkylene; and the remaining variables are as defined in the 1^{st}, 2^{nd}, or 3^{rd} embodiment or any specific embodiment described therein. In a specific embodiment, R⁵ is —(CH₂)₃—, —(CH₂)₅— or —(CH₂)₇—. In a more specific embodiment, R⁵ is —(CH₂)₇—. In a more specific embodiment, R⁵ is —(CH₂)₅—. In a more specific embodiment, R⁵ is —(CH₂)₃—.

In a 5^{th} embodiment, the compound of the present invention is represented by a formula described in the 1^{st} or 2^{nd} embodiment, or a pharmaceutically acceptable salt thereof, wherein R⁵ is represented by the following formula:

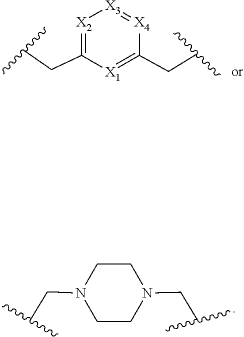

wherein X₁, X₂, X₃ and X₄ are each independently N or CR⁶, provided at least one of X₁, X₂, X₃ and X₄ is CR⁶; and the remaining variables are as defined in the 1^{st}, 2^{nd} or 3^{rd} embodiment or any specific embodiment described therein.

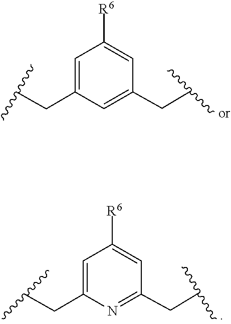

In a specific embodiment, R⁵ is

In a more specific embodiment, R⁵ is

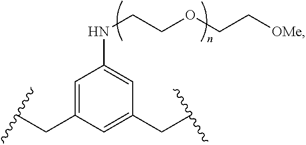

wherein n is an integer from 1 to 8. In a further specified embodiment, n is 1, 2, 3, or 4. In a more specific embodiment, n is 1. In a more specific embodiment, n is 2. In a more specific embodiment, n is 3. In a more specific embodiment, n is 4. In a more specific embodiment, R⁵ is

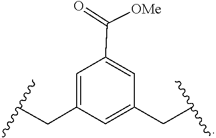 [SMILES: *Cc1cc(C*)cc(C(=O)OC)c1]

In a more specific embodiment, R⁵ is

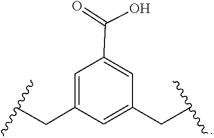 [SMILES: *Cc1cc(C*)cc(C(=O)O)c1]

In another specific embodiment, R⁵ is

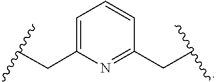 [SMILES: *Cc1cccc(C*)n1]

In a 6^{th} embodiment, the compound of the present invention is represented by one of the following formulae in Table B:

| | |
|---|---|
| 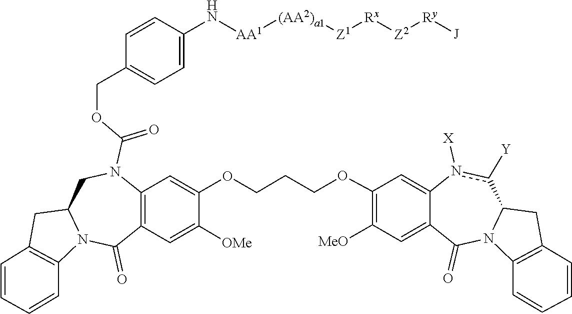 | (IIIaA) |
| 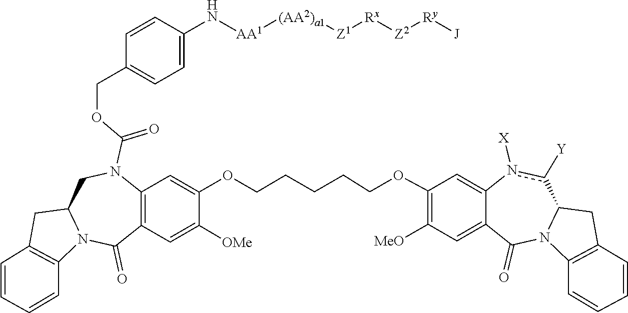 | (IIIaB) |
| 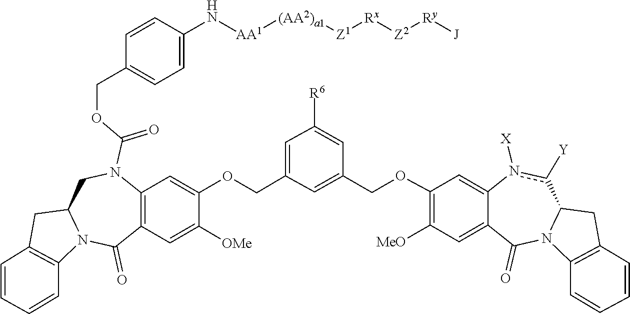 | (IIIaC) |
| 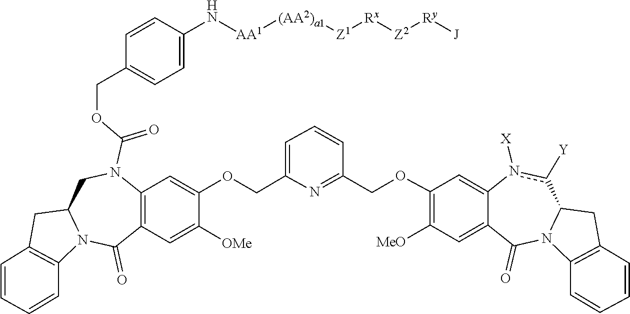 | (IIIaD) |
| 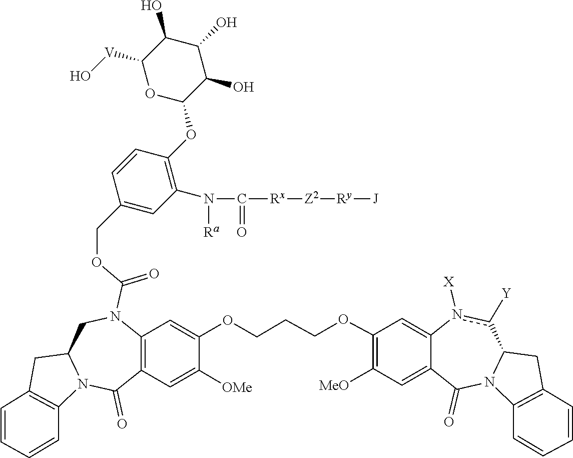 | (IIIbA) |
| 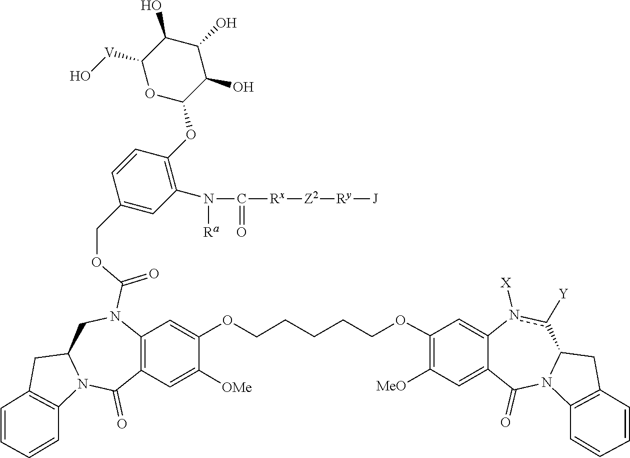 | (IIIbB) |
| 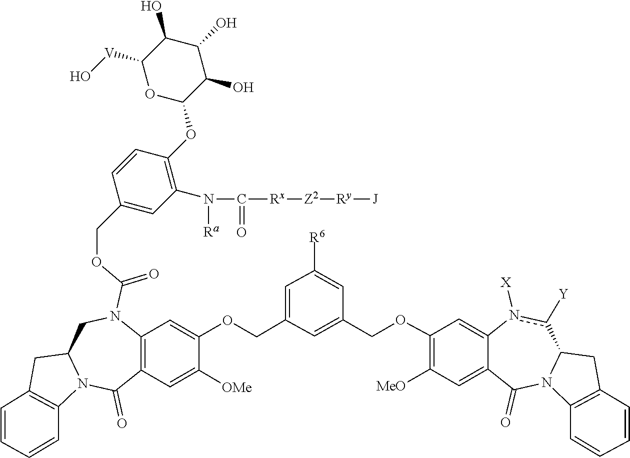 | (IIIbC) |
| 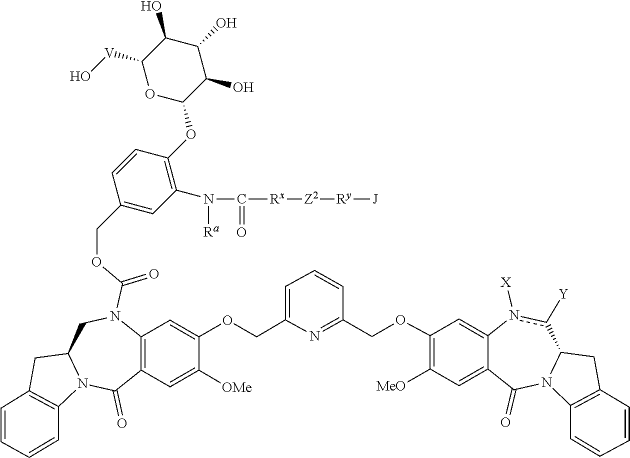 | (IIIbD) |
| 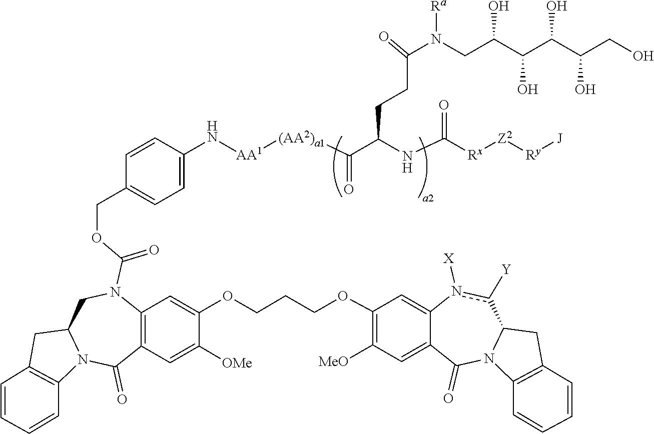 | (IIIcA) |
| 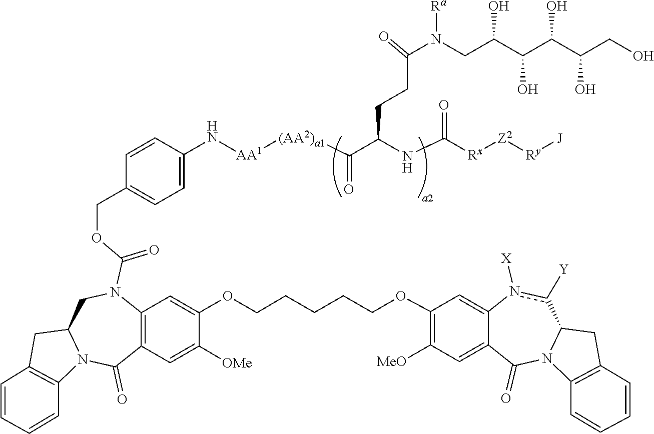 | (IIIcB) |
| 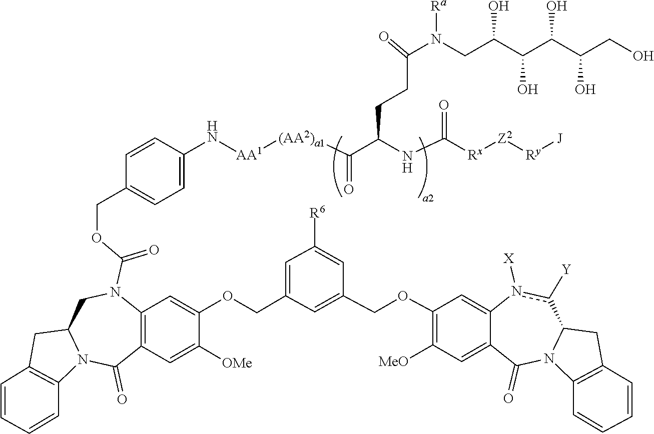 | (IIIcC) |
| 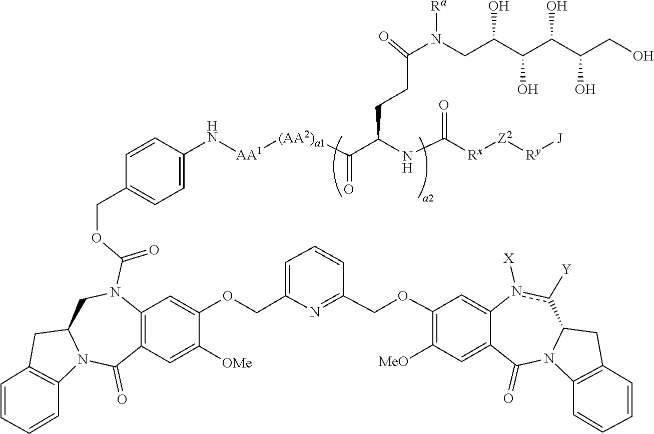 | (IIIcD) |
| 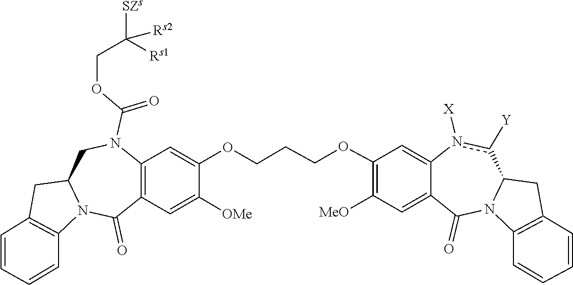 | (IIIdA) |
| 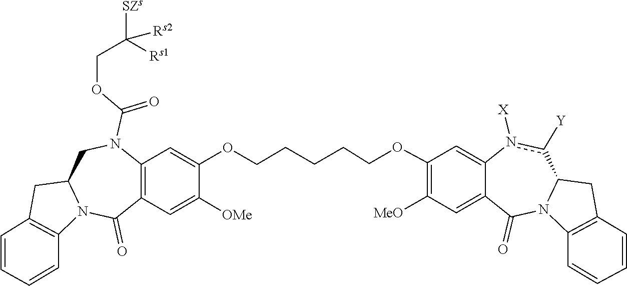 | (IIIdB) |
| 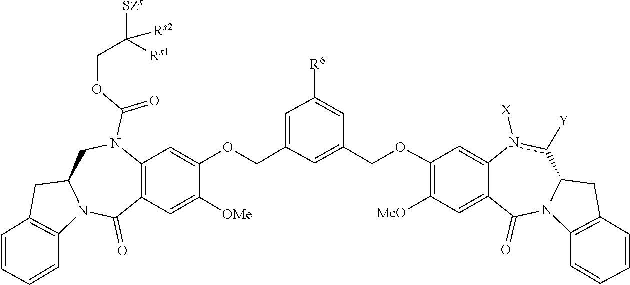 | (IIIdC) |
| 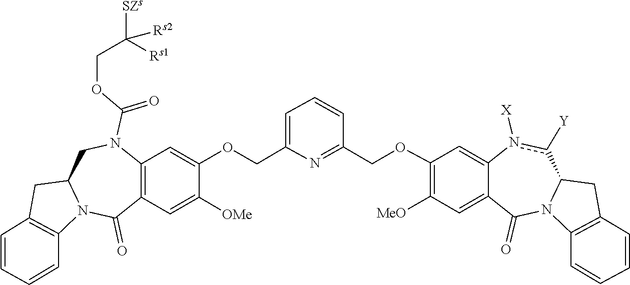 | (IIIdD) |
| 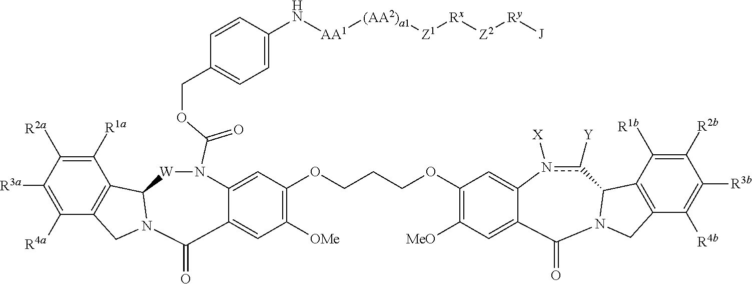 | (IVaA) |
| 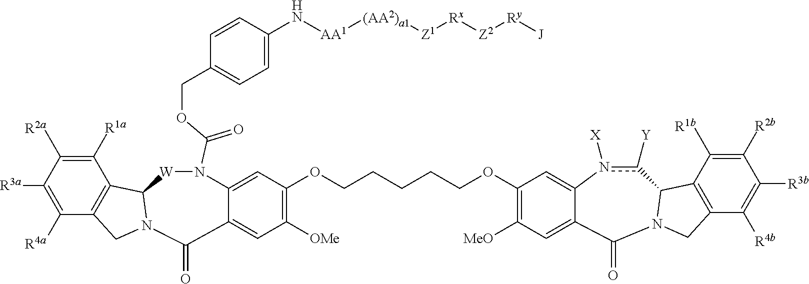 | (IVaB) |
| 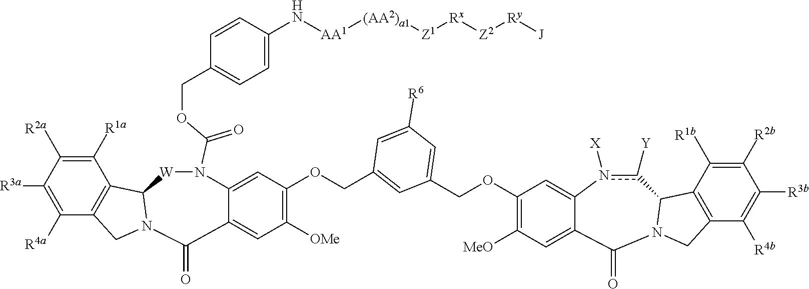 | (IVaC) |
| 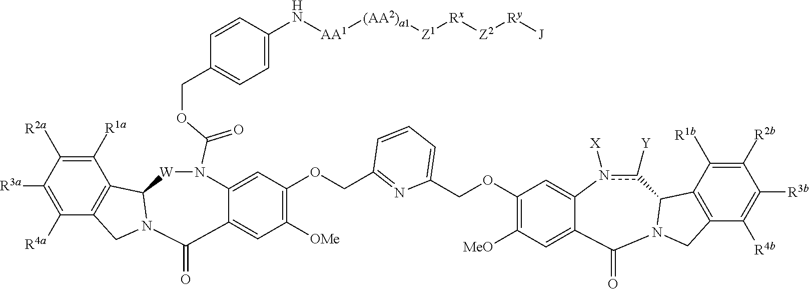 | (IVaD) |
| 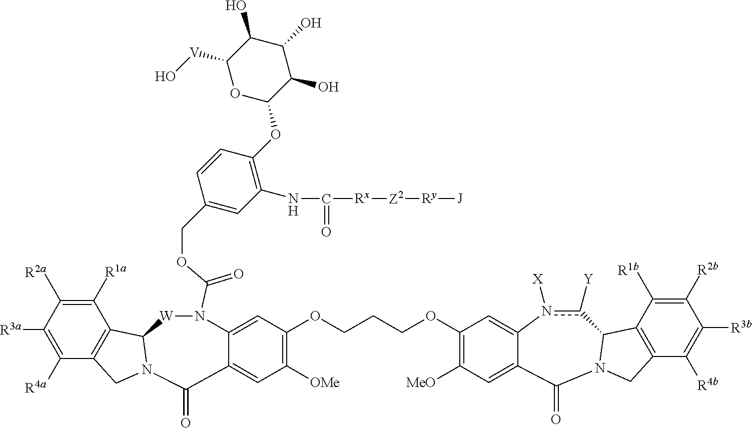 | (IVbA) |
| 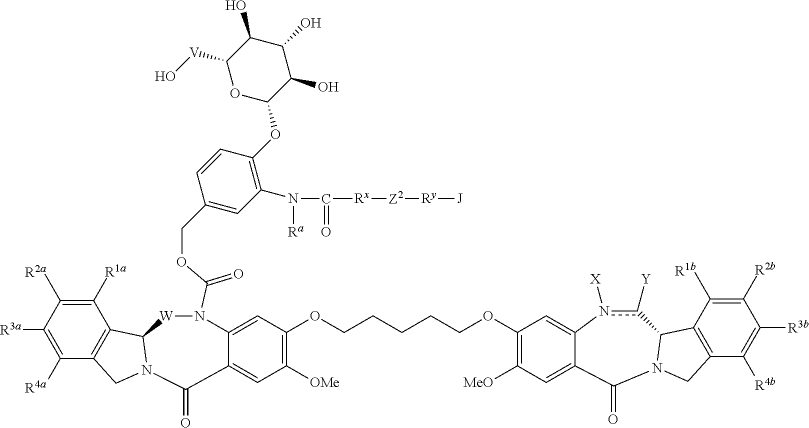 | (IVbB) |
| 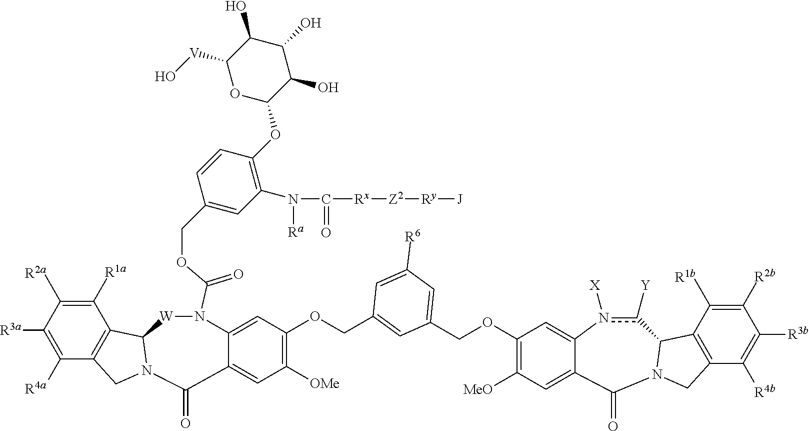 | (IVbC) |
| 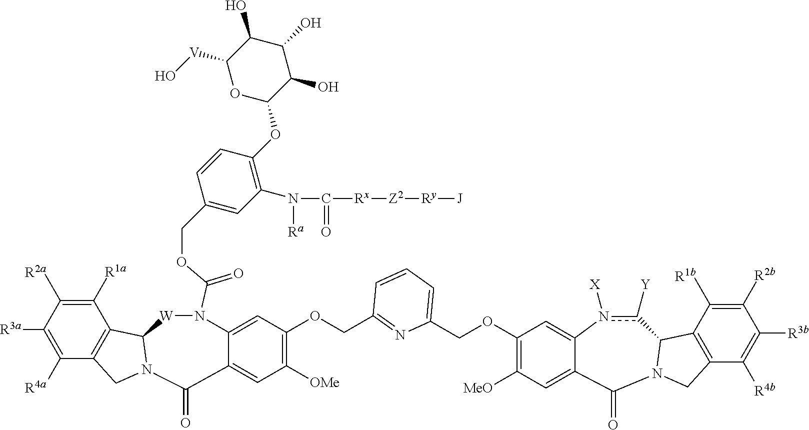 | (IVbD) |
| 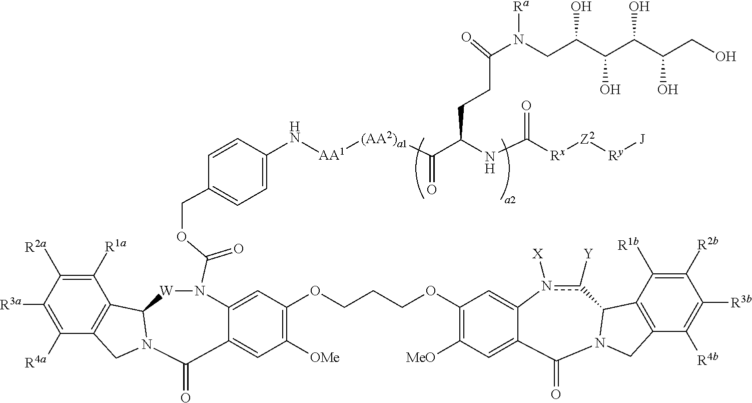 | (IVcA) |
| 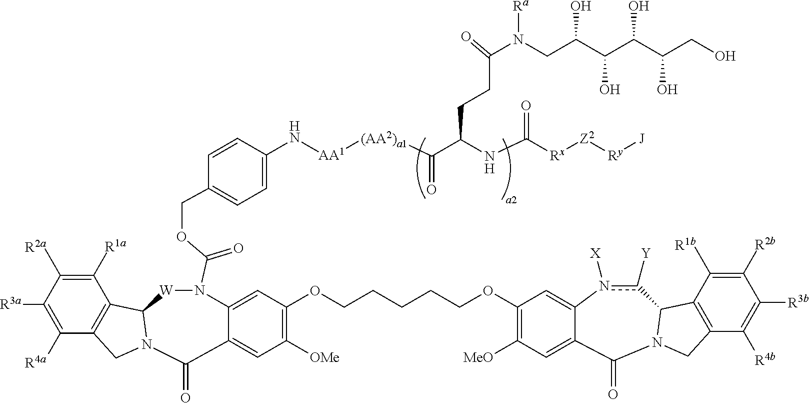 | (IVcB) |
| 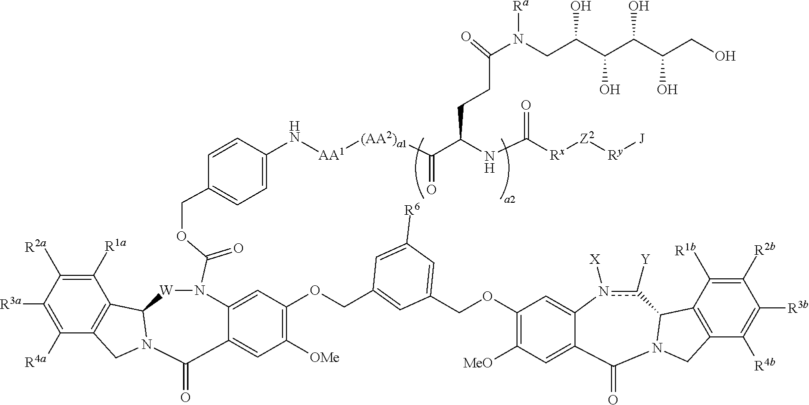 | (IVcC) |
| 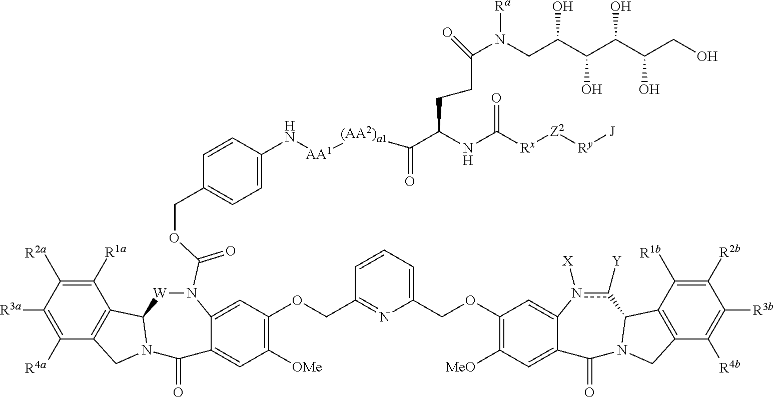 | (IVcD) |
| 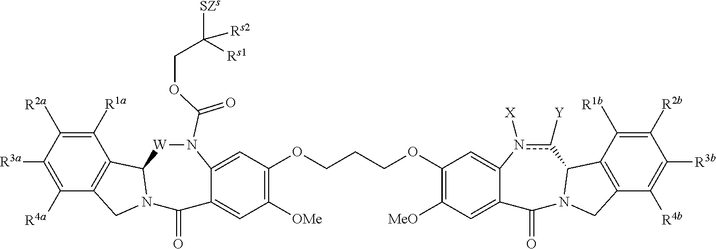 | (IVdA) |
| 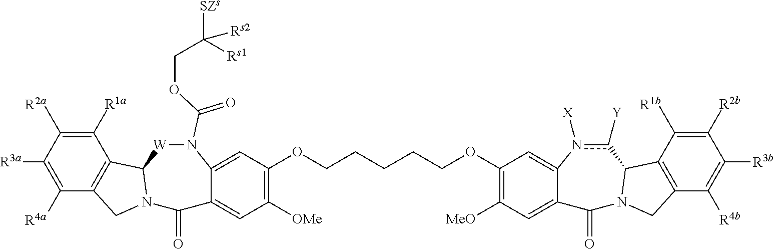 | (IVdB) |
| 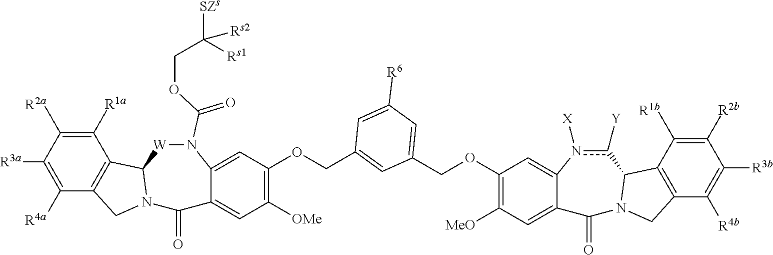 | (IVdC) |
| 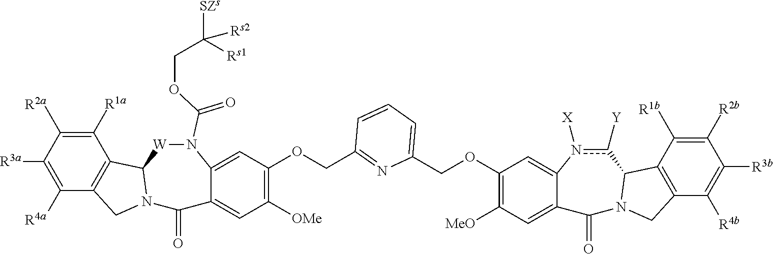 | (IVdD) |
| 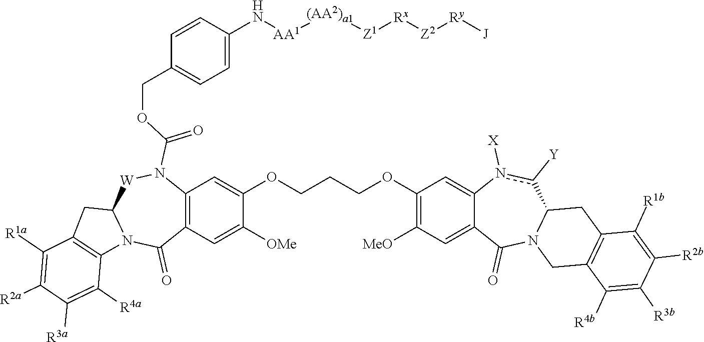 | (T1aA) |
| 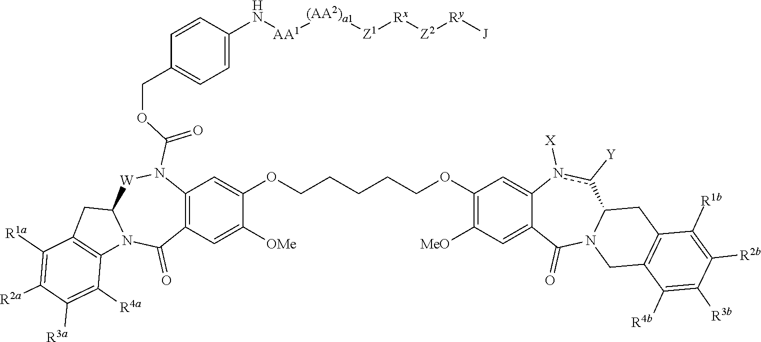 | (T1aB) |
| 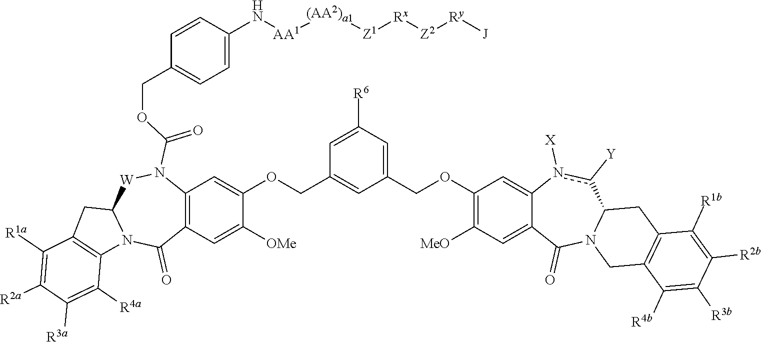 | (T1aC) |
| 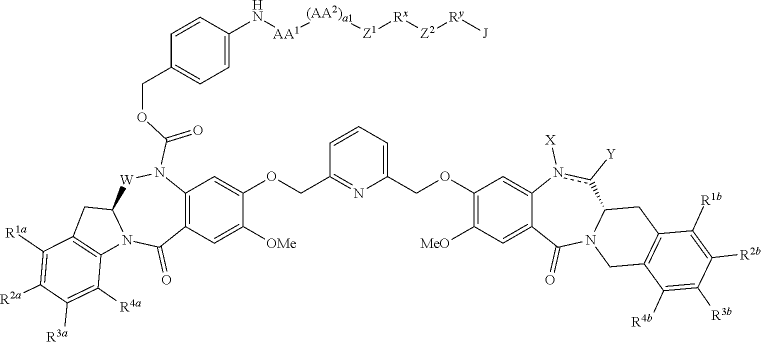 | (T1aD) |
| 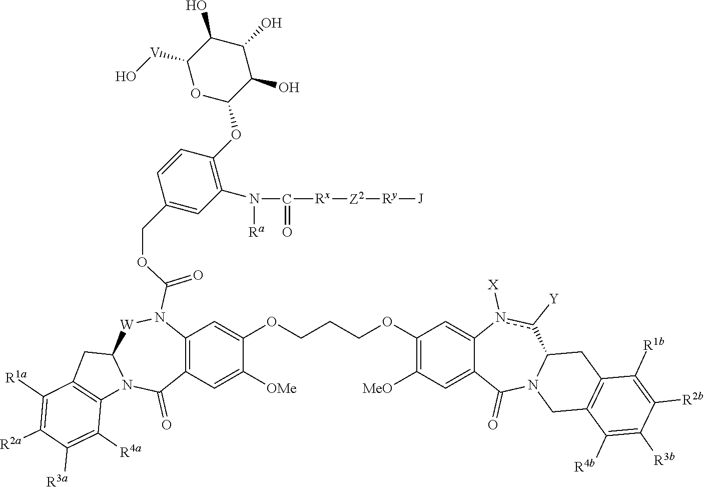 | (TIbA) |
| 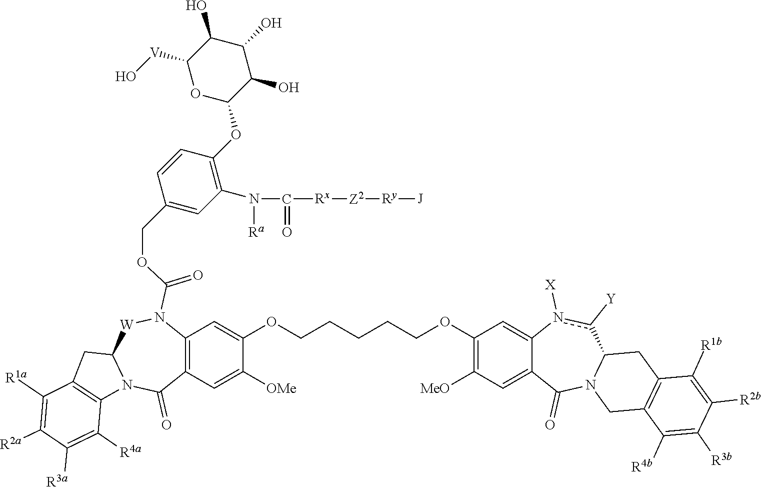 | (TIbB) |
| 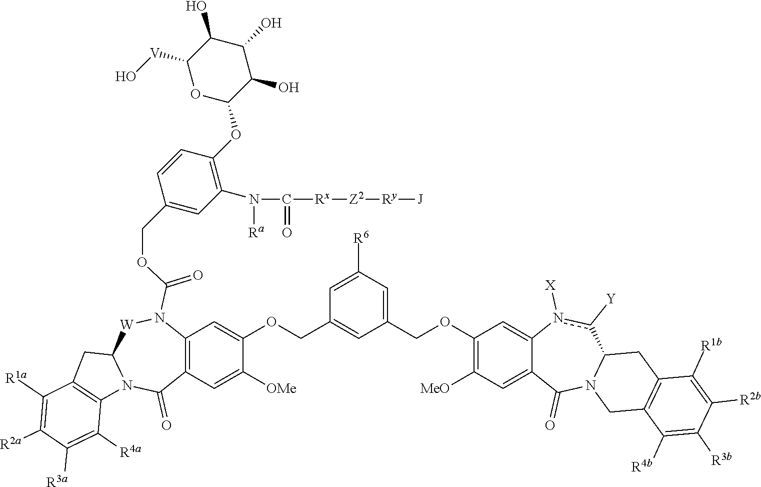 | (TIbC) |
| 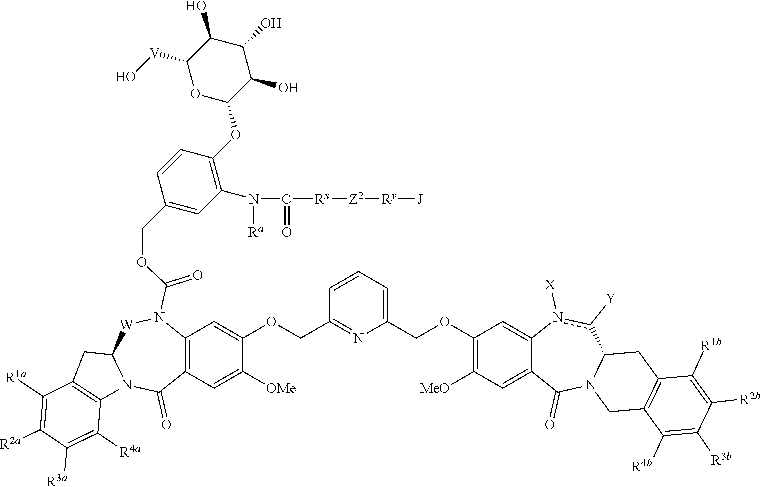 | (TIbD) |
| 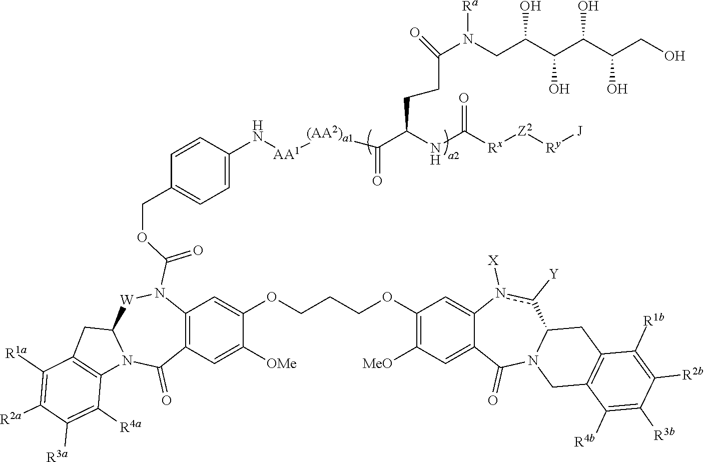 | (T1cA) |
| 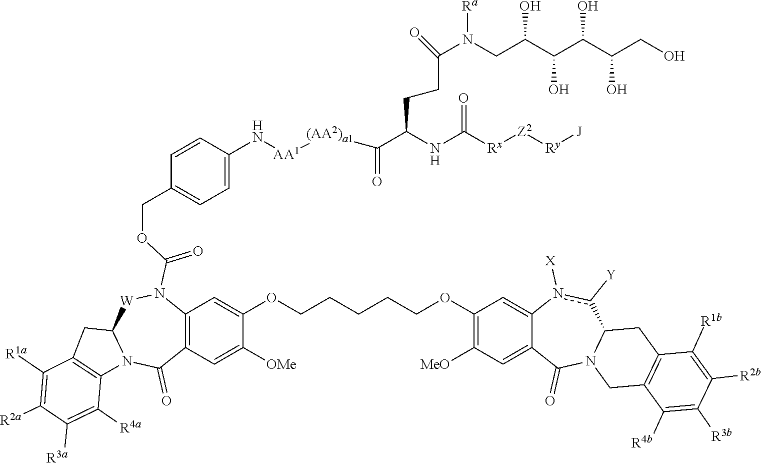 | (T1cB) |
| 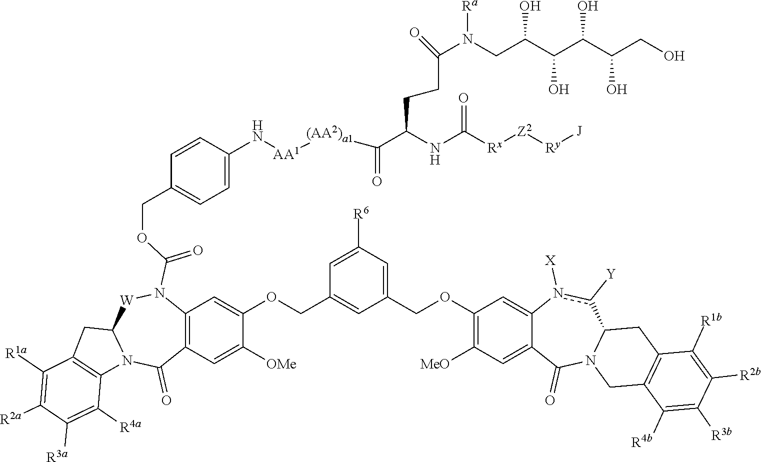 | (T1cC) |
| 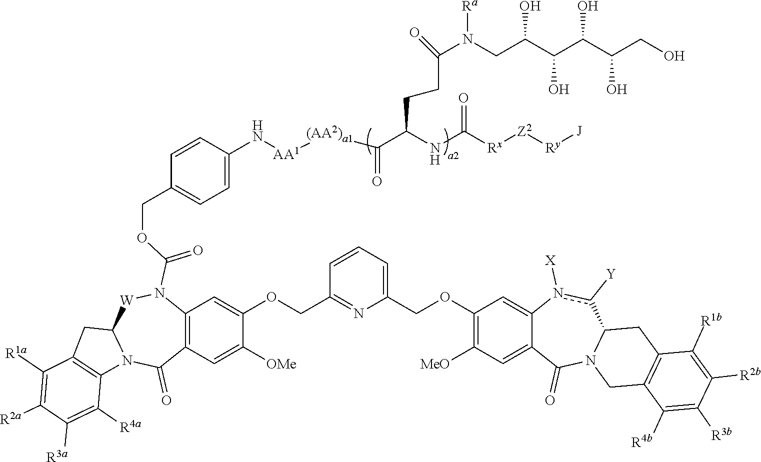 | (T1cD) |
| 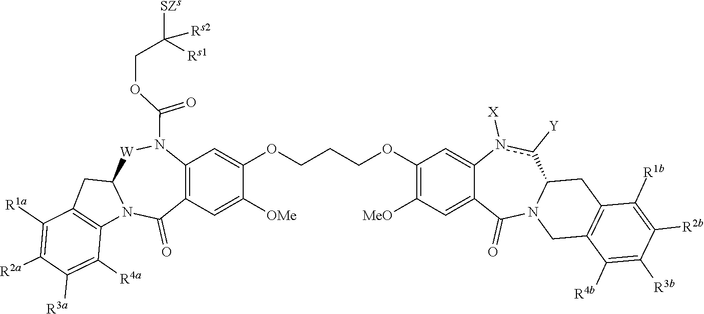 | (T1dA) |
| 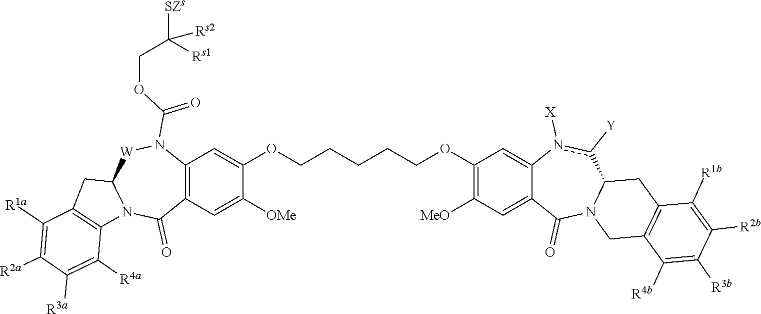 | (T1dB) |
| 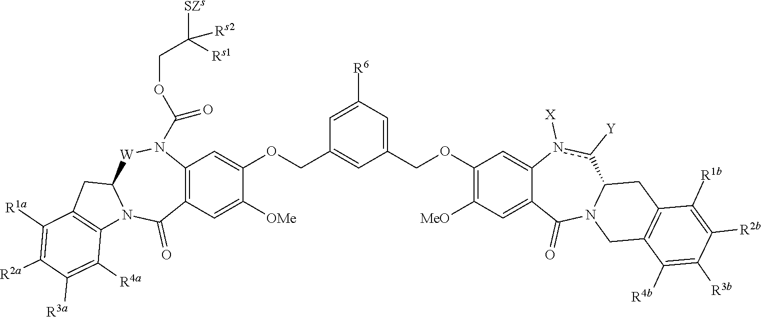 | (T1dC) |
| 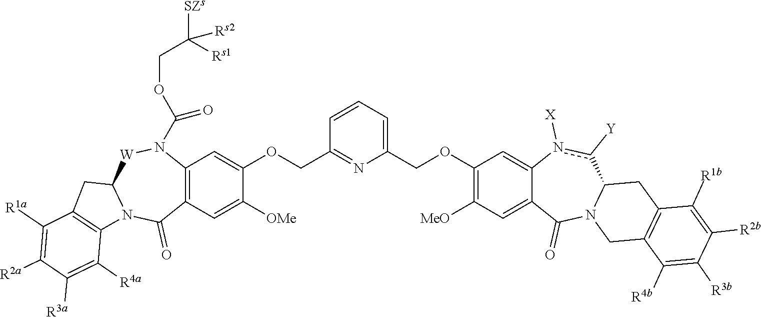 | (T1dD) |
| 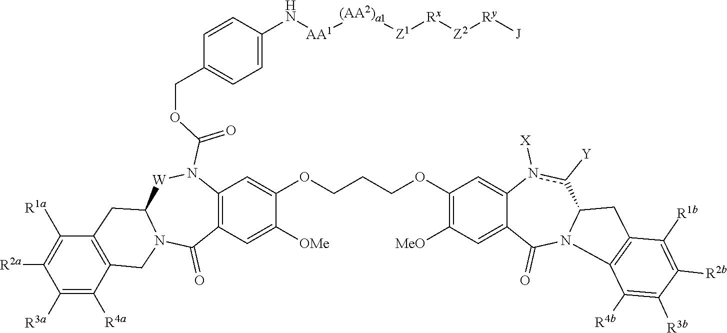 | (T2aA) |
| 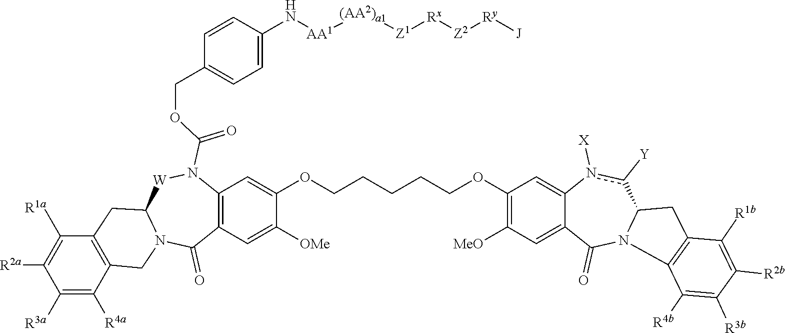 | (T2aB) |
| 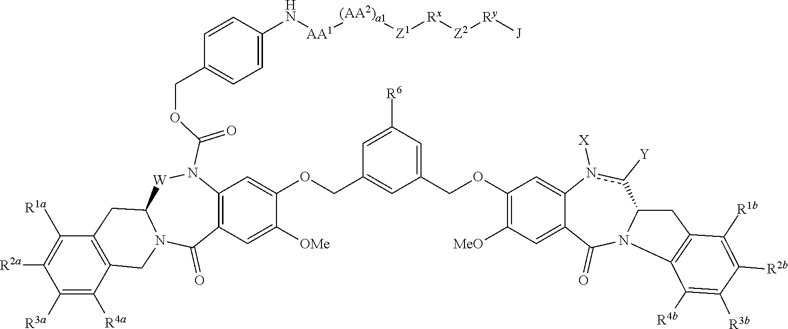 | (T2aC) |
| 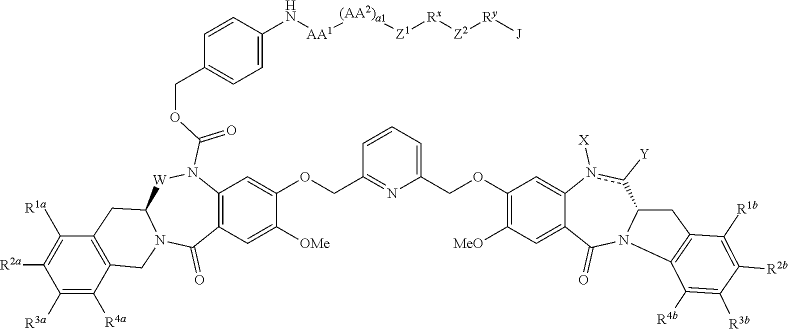 | (T2aD) |
| 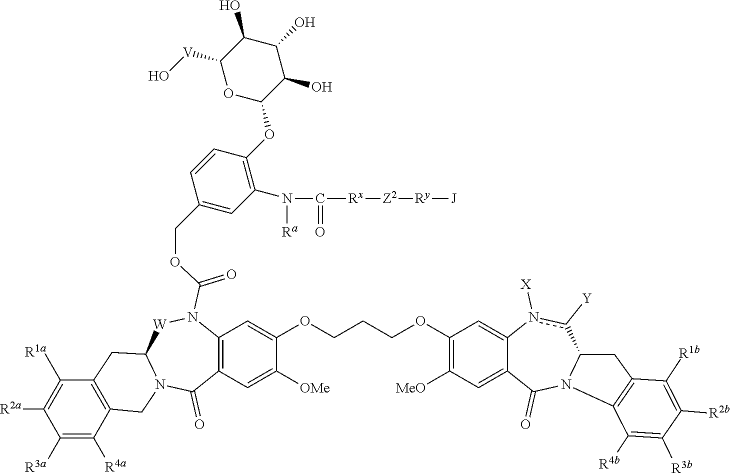 | (T2bA) |
| 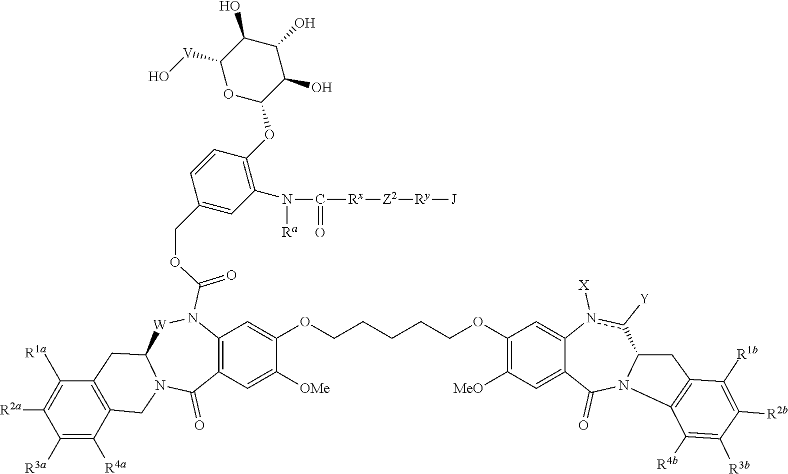 | (T2bB) |
| 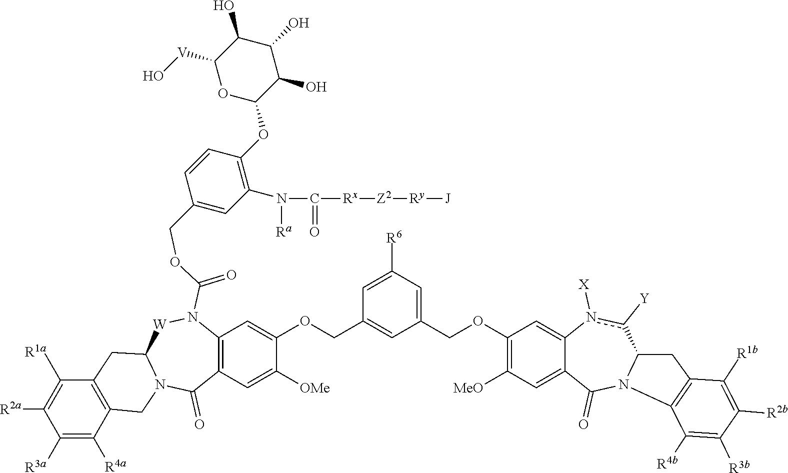 | (T2bC) |
| 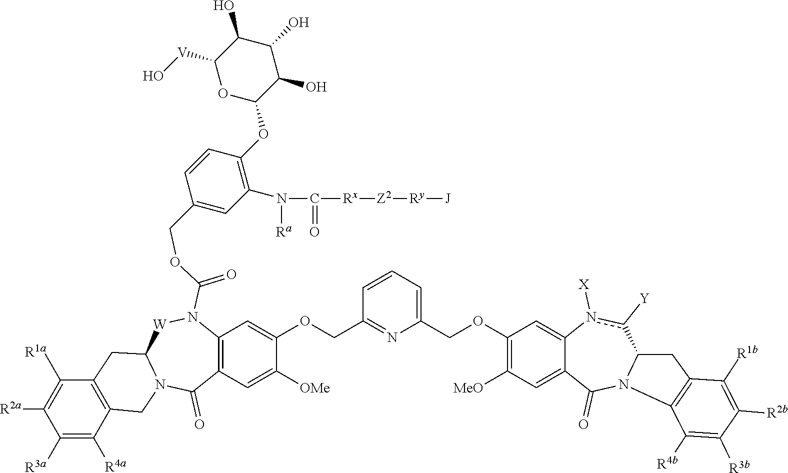 | (T2bD) |
| 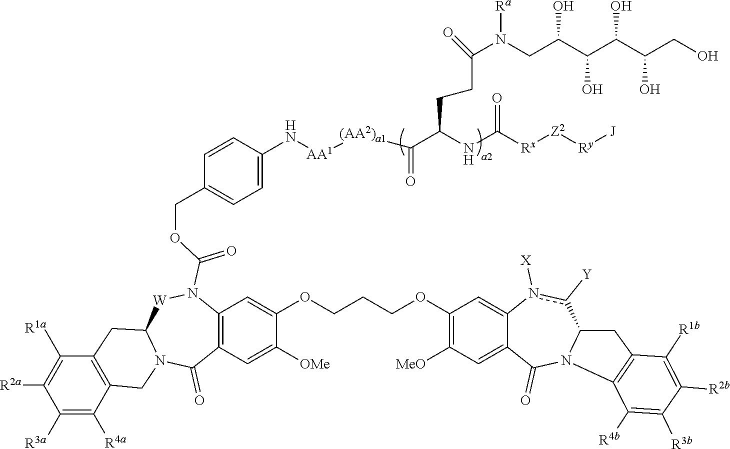 | (T2cA) |
| 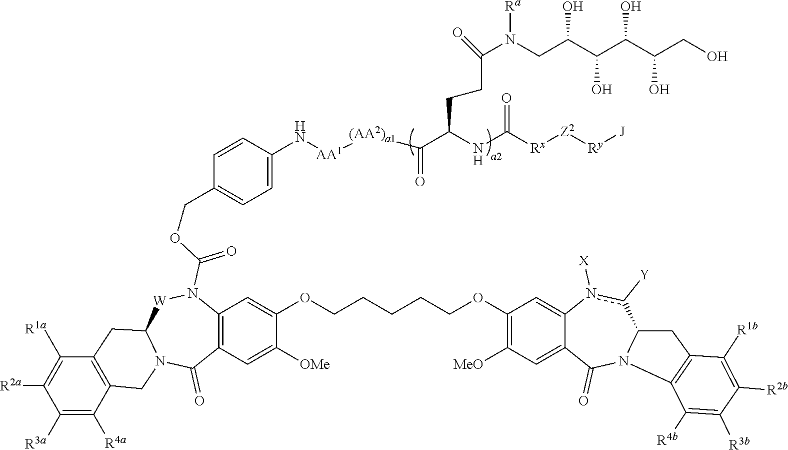 | (T2cB) |
| 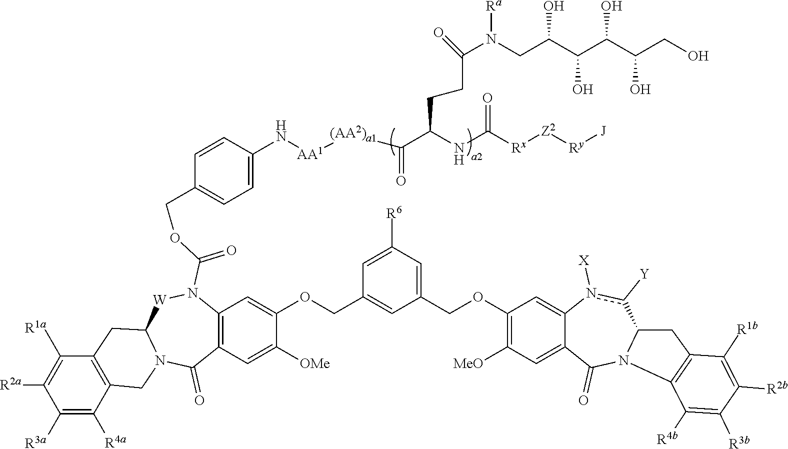 | (T2cC) |
| 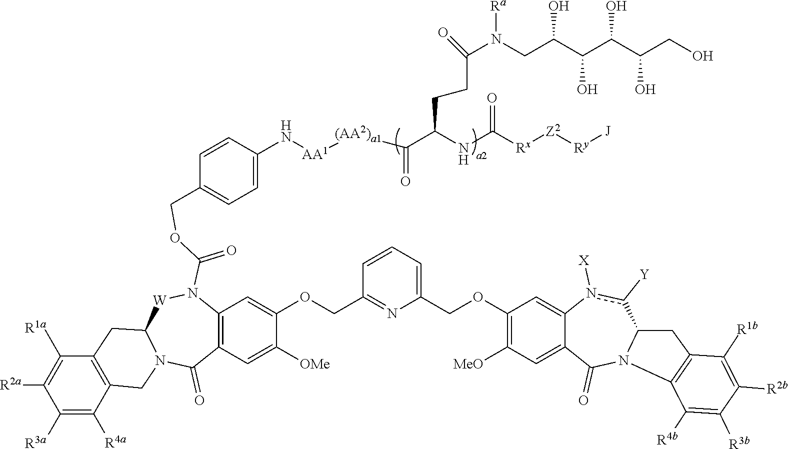 | (T2cD) |
| 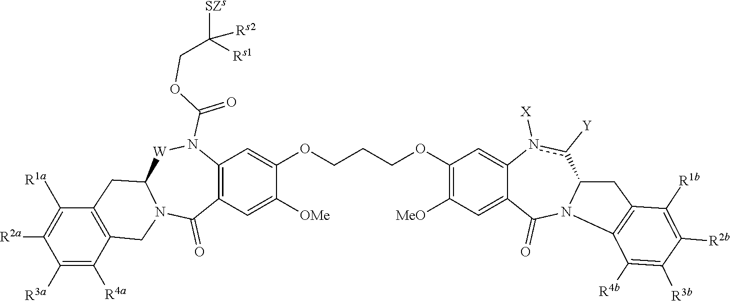 | (T2dA) |
| 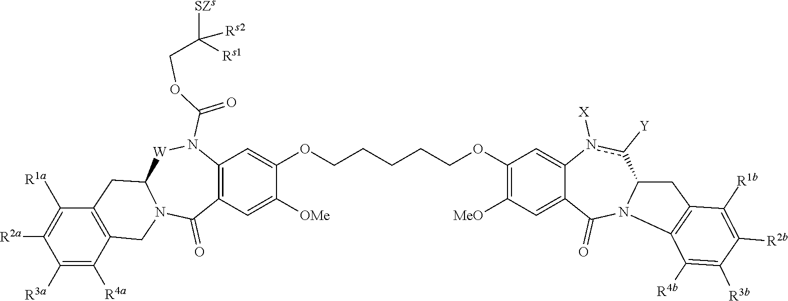 | (T2dB) |
| 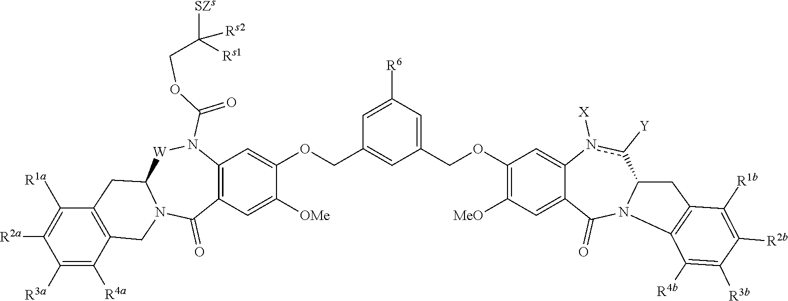 | (T2dC) |
| 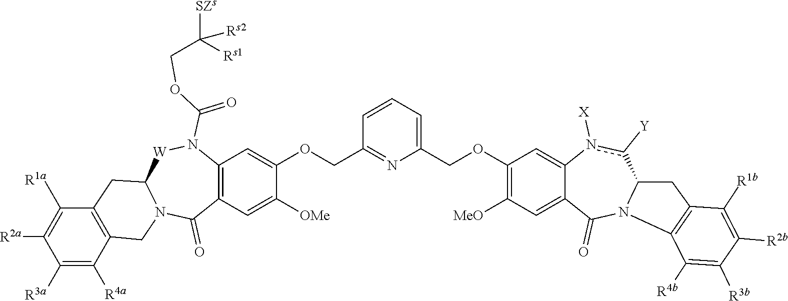 | (T2dD) |

or a pharmaceutically acceptable salt thereof, wherein the remaining variables are as defined in the 2^{nd} embodiment or any specific embodiments described therein.

In a 7^{th} embodiment, the compound of the present invention is represented by a formula described in the 2^{nd} or 6^{th} embodiment, or a pharmaceutically acceptable salt thereof, wherein Z¹ is —C(═O)—; and the remaining variables are as defined in the 2^{nd}, 3^{rd}, 4^{th}, 5^{th} or 6^{th} embodiment or any specific embodiments described therein.

In a 8^{th} embodiment, the compound of the present invention is represented by a formula recited in the 2^{nd} or 6^{th} embodiment, or a pharmaceutically acceptable salt thereof, wherein R^{x} is C₁₋₆alkylene; Z² and R^{y} are both absent; and the remaining variables are as defined in the 2^{nd}, 3^{rd}, 4^{th}, 5^{th}, 6^{th} or 7^{th} embodiment or any specific embodiment described therein. In another embodiment, R^{x}, Z² and R^{y} are absent; and the remaining variables are as defined in the 2^{nd}, 3^{rd}, 4^{th}, 5^{th}, 6^{th} or 7^{th} embodiment or any specific embodiment described therein.

In a 9^{th} embodiment, the compound of the present invention is represented by a formula described in the 2^{nd} or 6^{th} embodiment, or a pharmaceutically acceptable salt thereof, wherein R^{x} is —(CH₂CH₂O)ₘ₁—C₁₋₆alkylene-; Z² is —NH—C(═O)— or —C(═O)—NH—; R^{y} is C₁₋₆alkylene; and the remaining variables are as defined in the 2^{nd}, 3^{rd}, 4^{th}, 5^{th}, 6^{th}, or 7^{th} embodiment or any specific embodiment described therein.

In a 10^{th} embodiment, the compound of the present invention is represented by a formula recited in the 2^{nd} or 6^{th} embodiment, or a pharmaceutically acceptable salt thereof, wherein R^{x} is C₁₋₆alkylene; Z² is —NH—C(═O)— or —C(═O)—NH—; R^{y} is —(CH₂CH₂O)ₘ₂—C₁₋₆alkylene-; and the remaining variables are as defined in the 2^{nd}, 3^{rd}, 4^{th}, 5^{th}, 6^{th} or 7^{th} embodiment or any specific embodiment described therein.

In a 11^{th} embodiment, the compound of the present invention is represented by one of the following formulae in Table C:

| | |
|---|---|
| 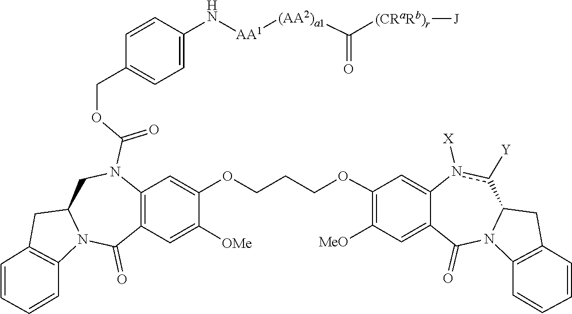 | (IIIa1A) |
| 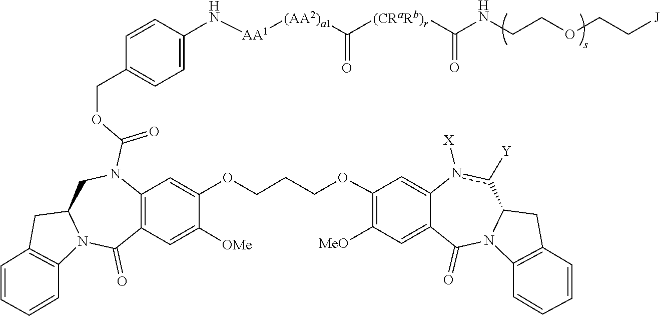 | (IIIa2A) |
| 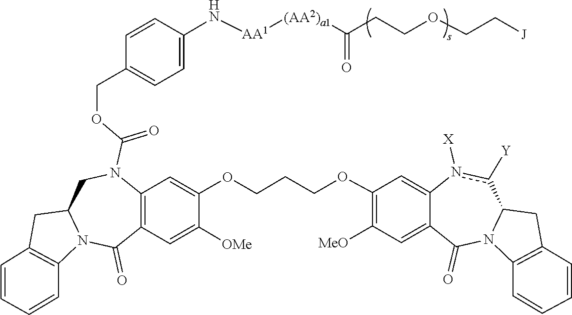 | (IIIa3A) |
| 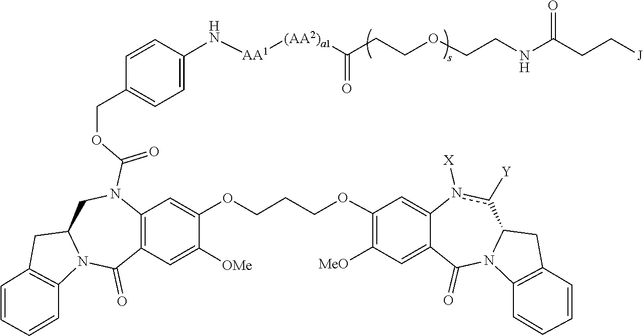 | (IIIa4A) |
| 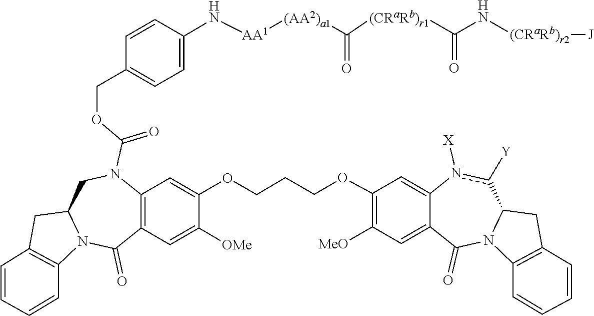 | (IIIa5A) |
| 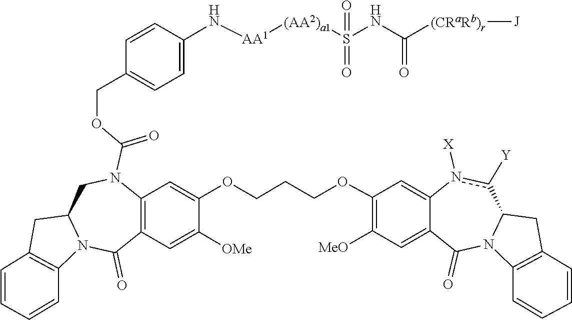 | (IIIa6A) |
| 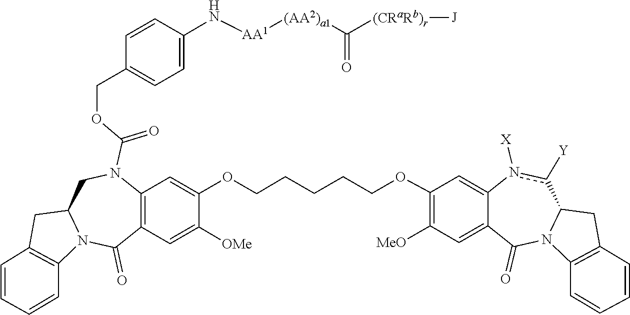 | (IIIa1B) |
| 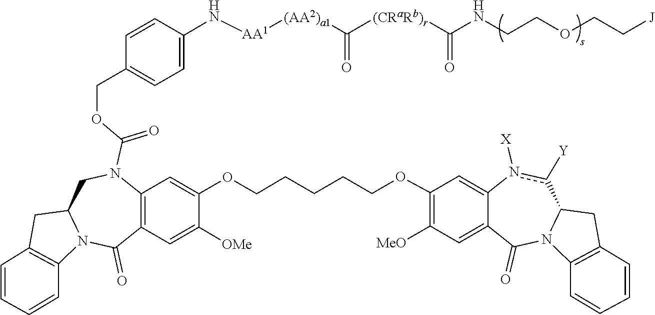 | (IIIa2B) |
| 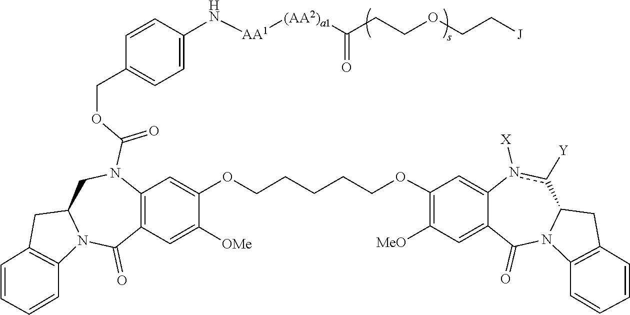 | (IIIa3B) |
| 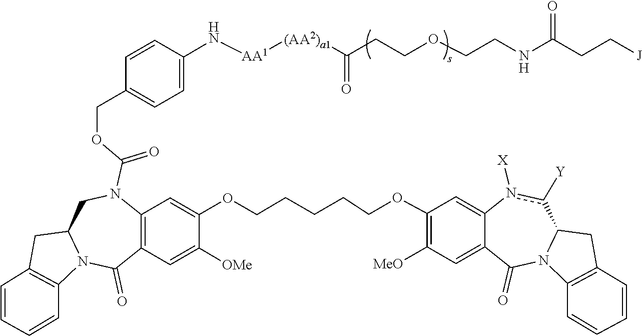 | (IIIa4B) |
| 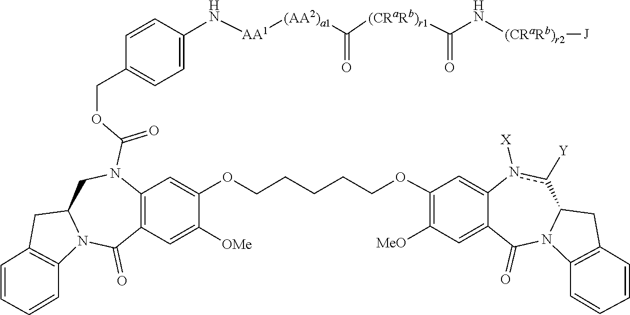 | (IIIa5B) |
| 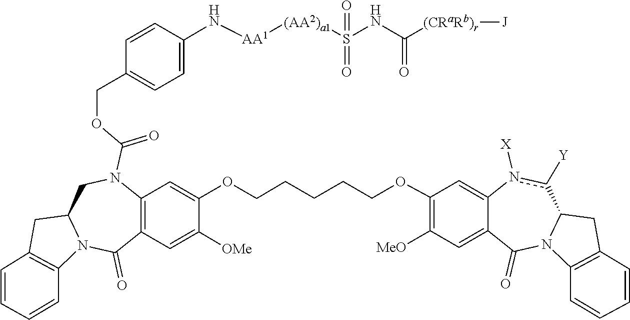 | (IIIa6B) |
| 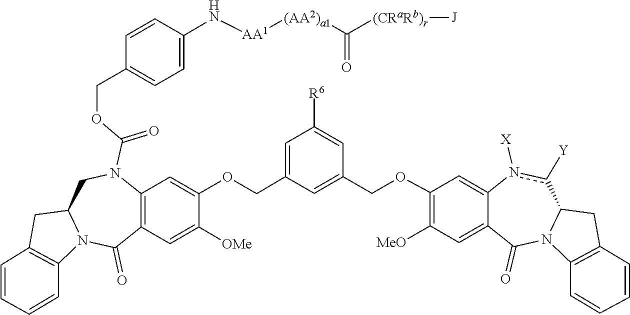 | (IIIa1C) |
| 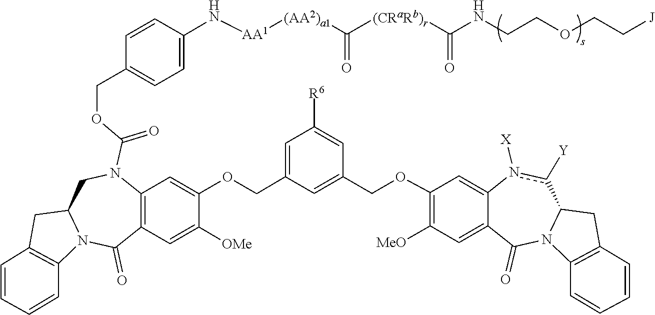 | (IIIa2C) |
| 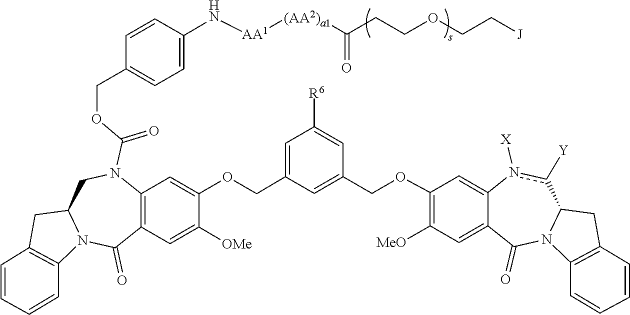 | (IIIa3C) |
| 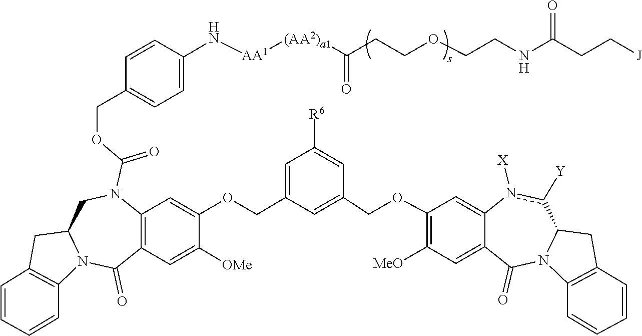 | (IIIa4C) |
| 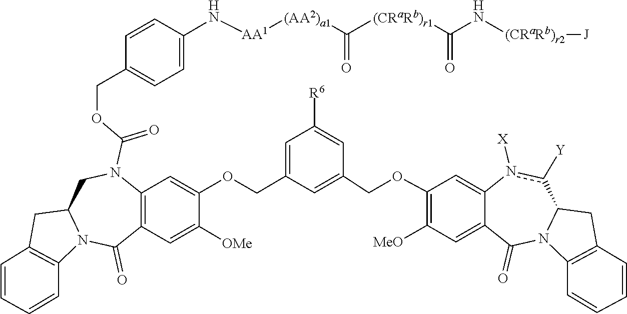 | (IIIa5C) |
| 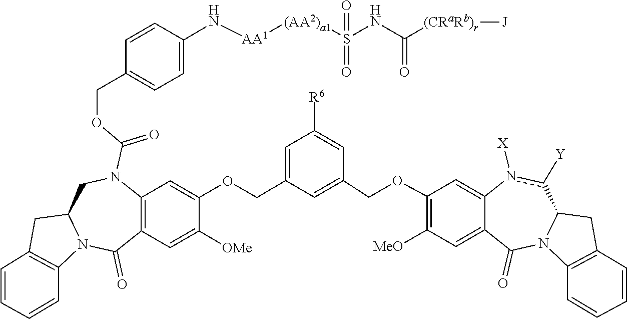 | (IIIa6C) |
| 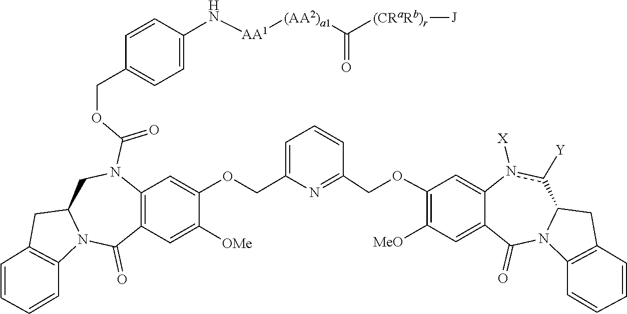 | (IIIa1D) |
| 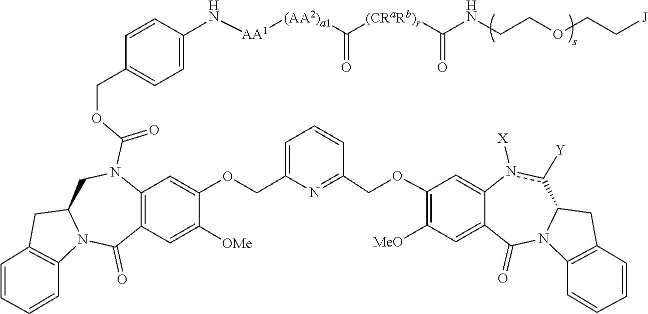 | (IIIa2D) |
| 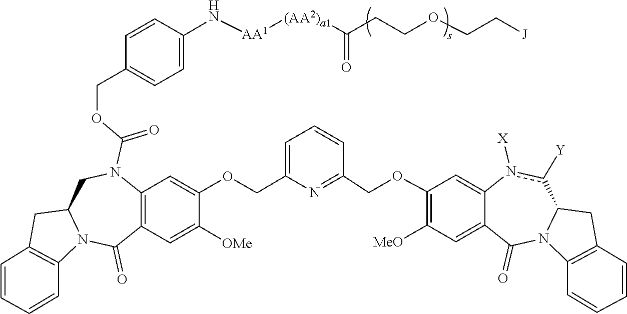 | (IIIa3D) |
| 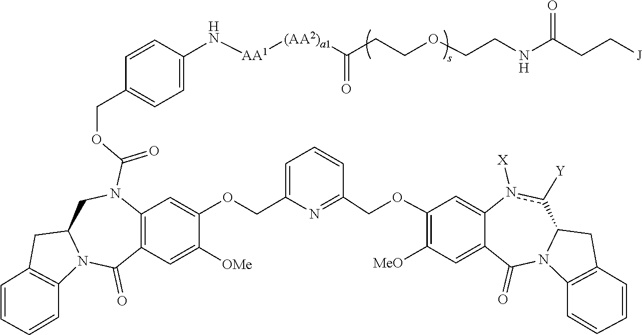 | (IIIa4D) |
| 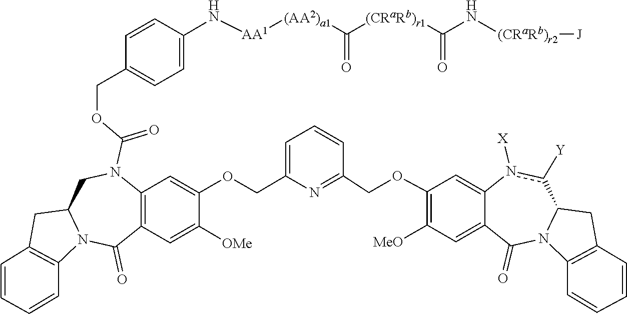 | (IIIa5D) |
| 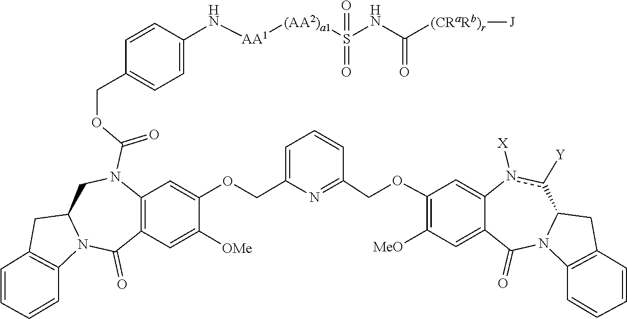 | (IIIa6D) |
| 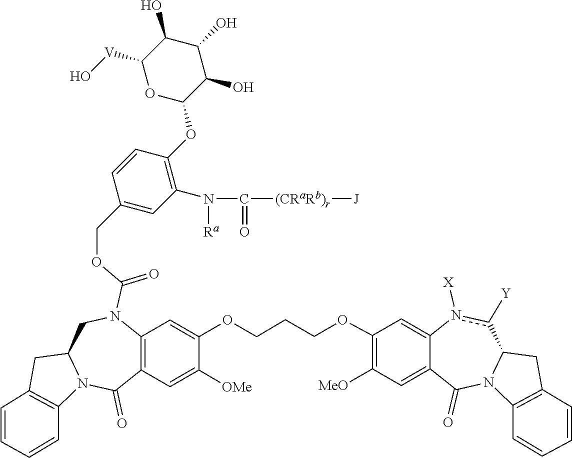 | (IIIb1A) |
| 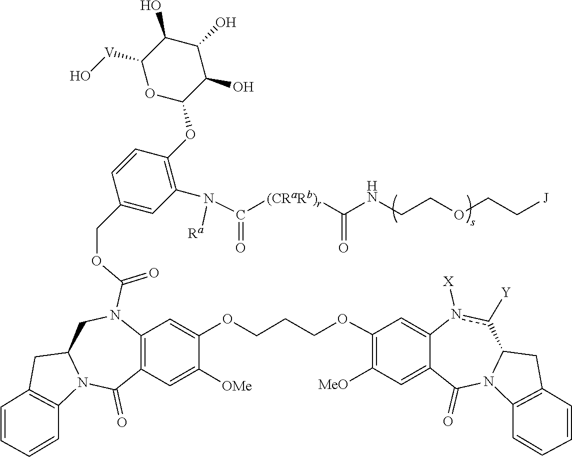 | (IIIb2A) |
| 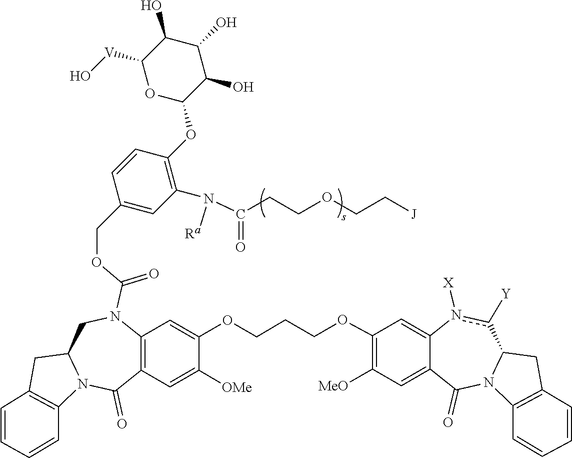 | (IIIb3A) |
| 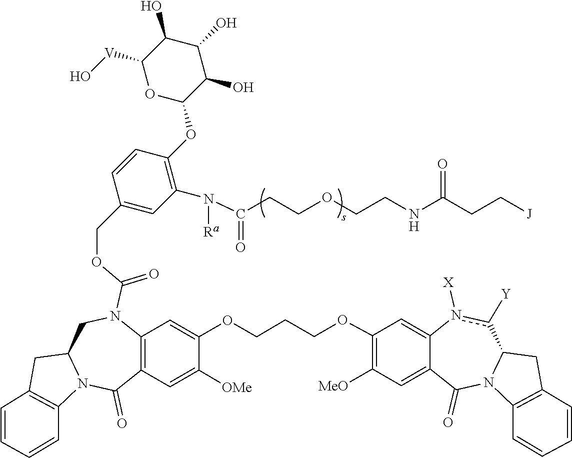 | (IIIb4A) |
| 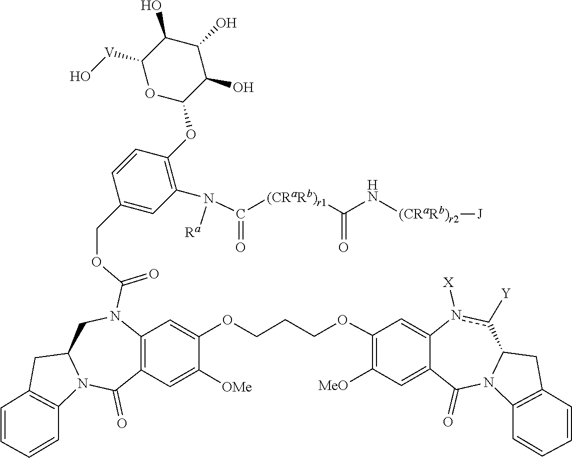 | (IIIb5A) |
| 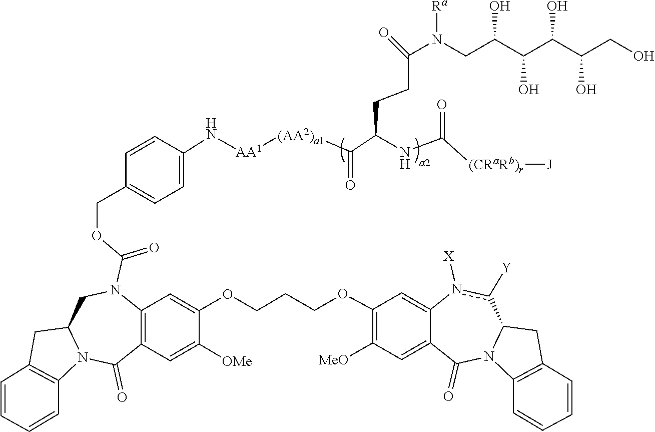 | (IIIc1A) |
| 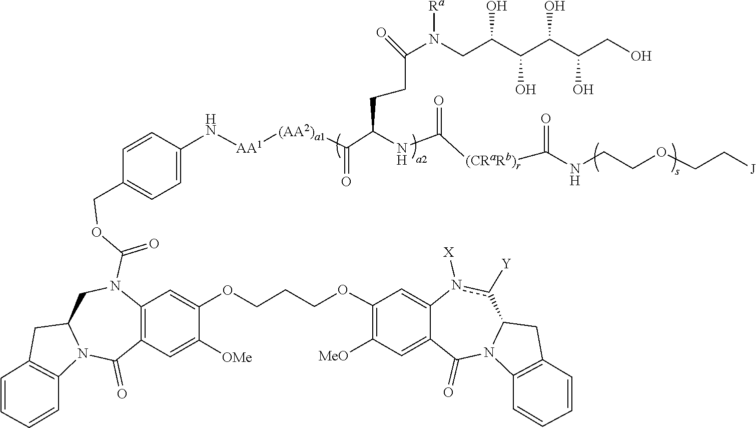 | (IIIc2A) |
| 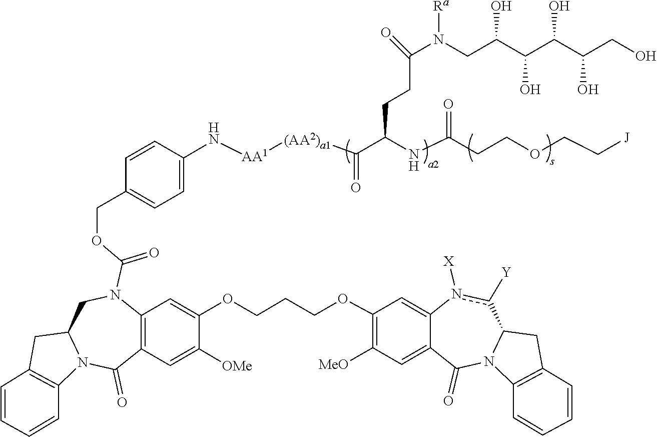 | (IIIc3A) |
| 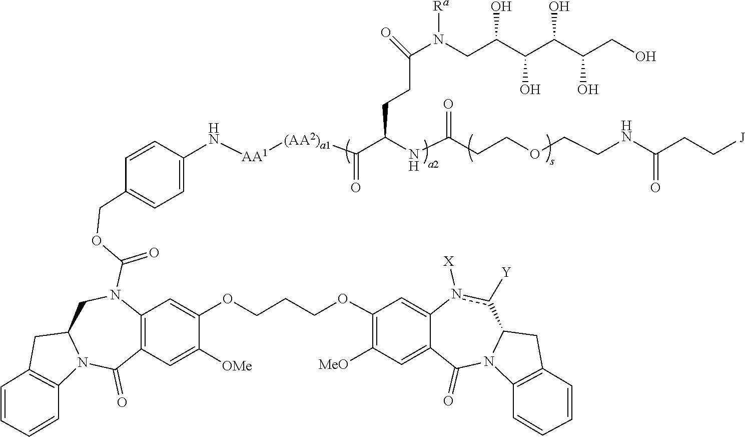 | (IIIc4A) |
| 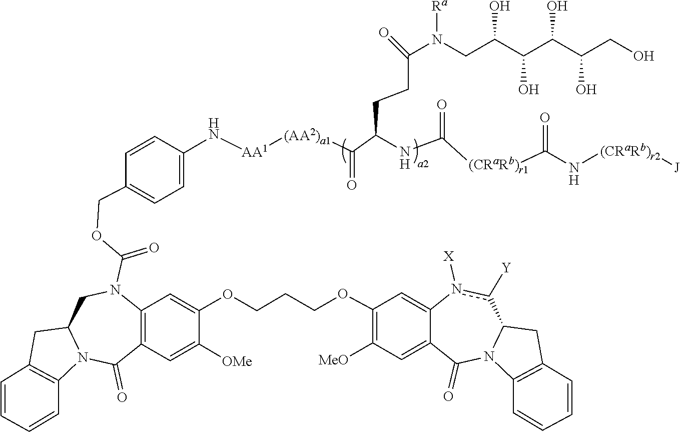 | (IIIc5A) |
| 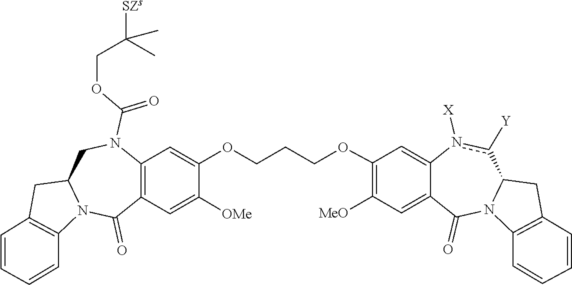 | (IIId1A) |
| 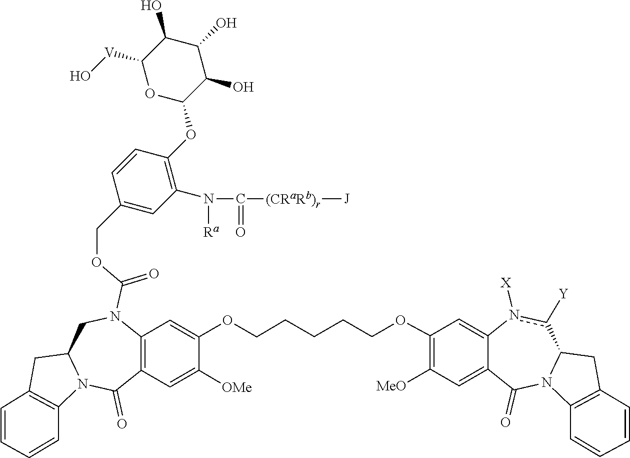 | (IIIb1B) |
| 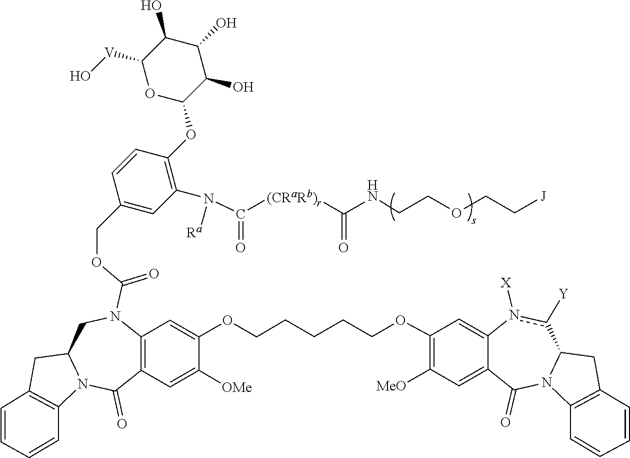 | (IIIb2B) |
| 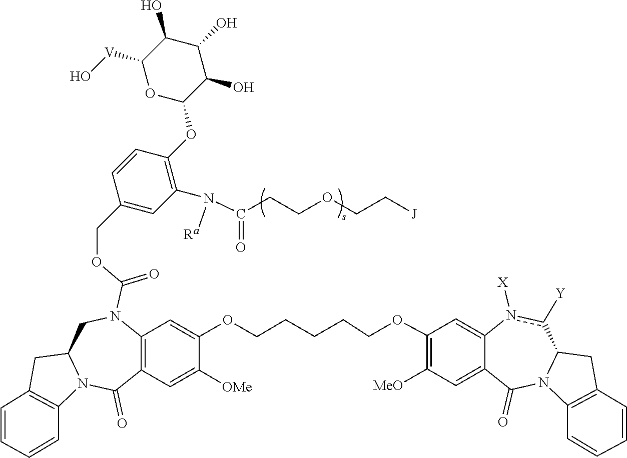 | (IIIb3B) |
| 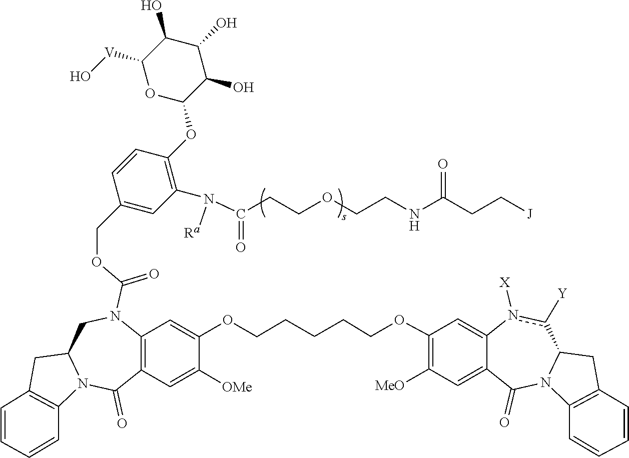 | (IIIb4B) |
| 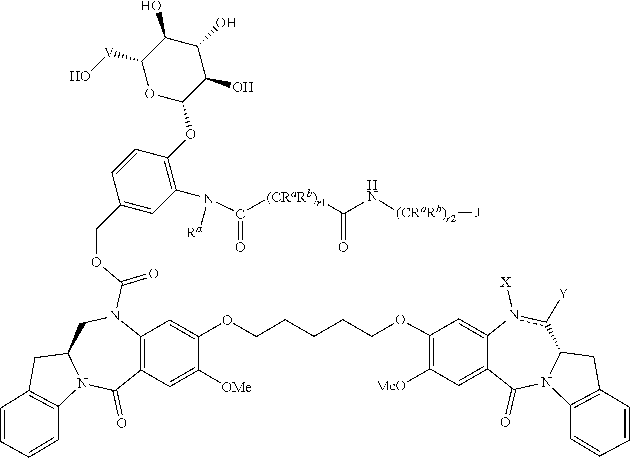 | (IIIb5B) |
| 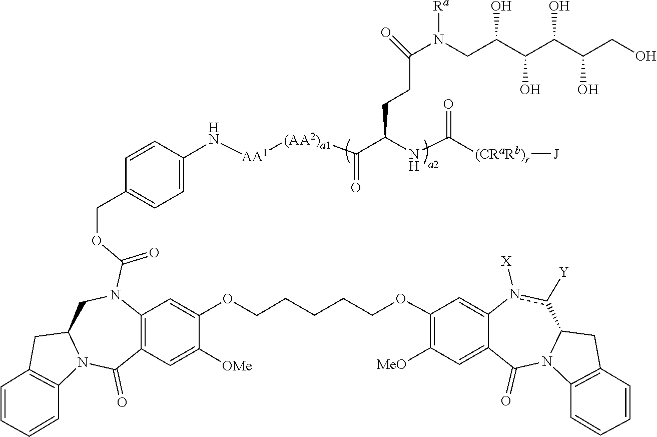 | (IIIc1B) |
| 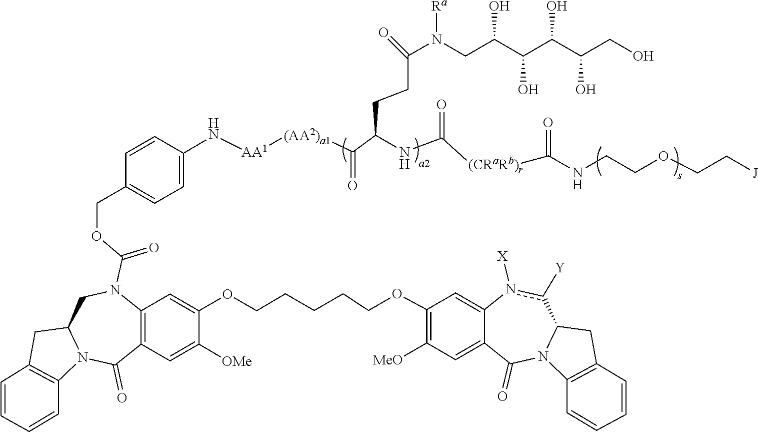 | (IIIc2B) |
| 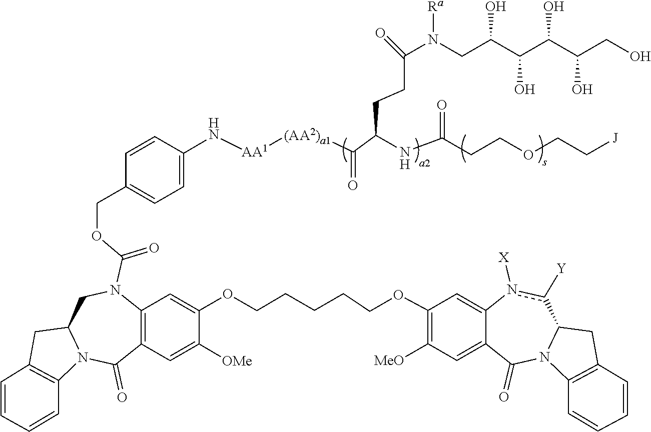 | (IIIc3B) |
| 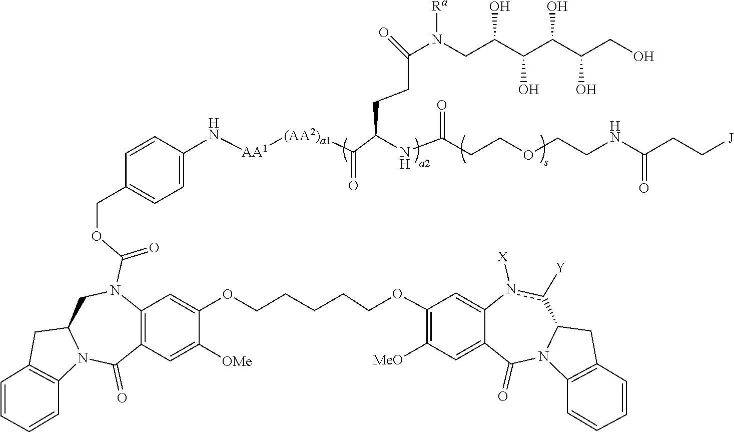 | (IIIc4B) |
| 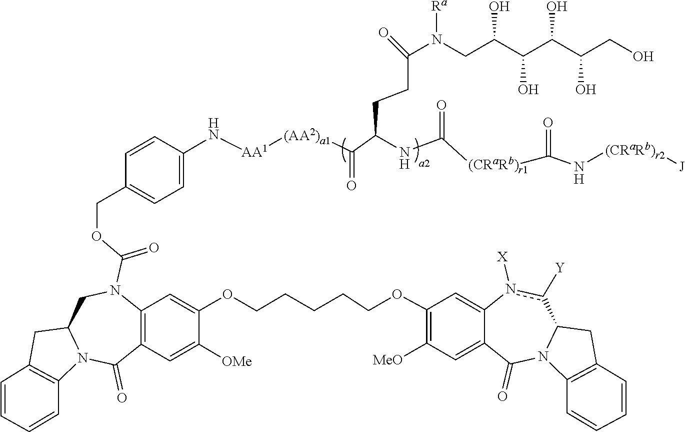 | (IIIc5B) |
| 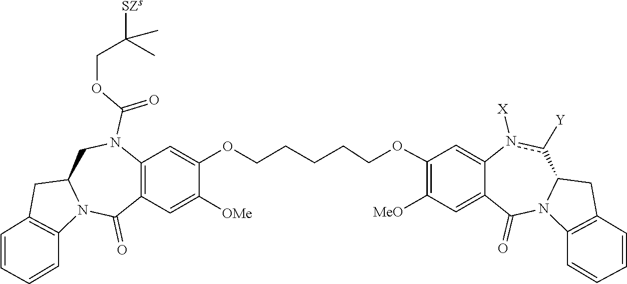 | (IIId1B) |
| 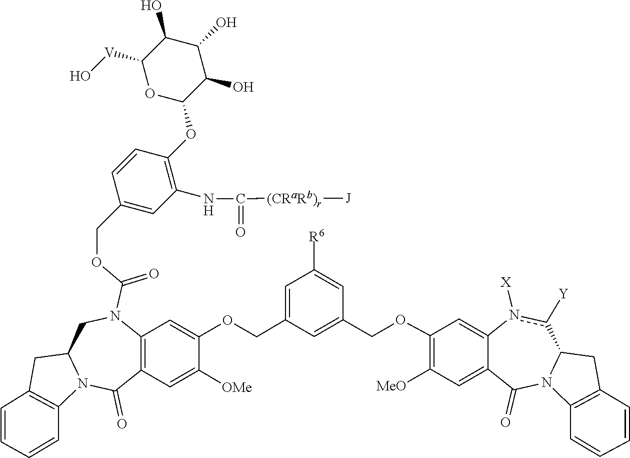 | (IIIb1C) |
| 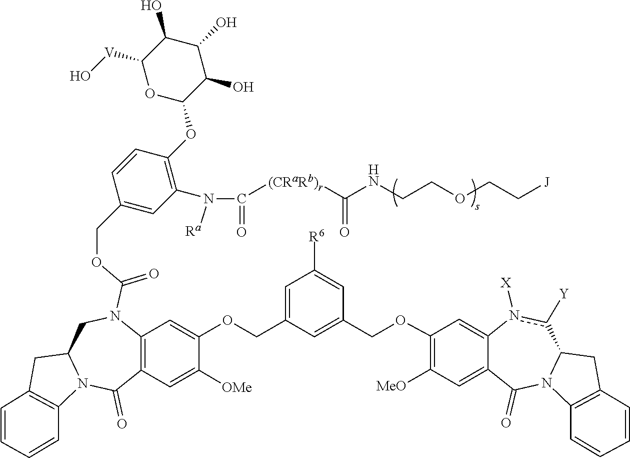 | (IIIb2C) |
| 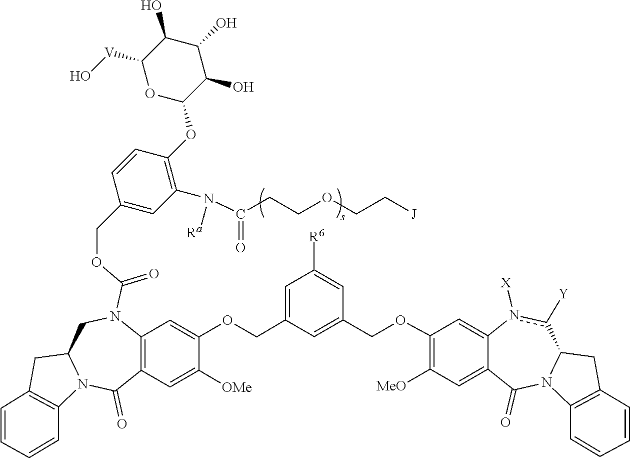 | (IIIb3C) |
| 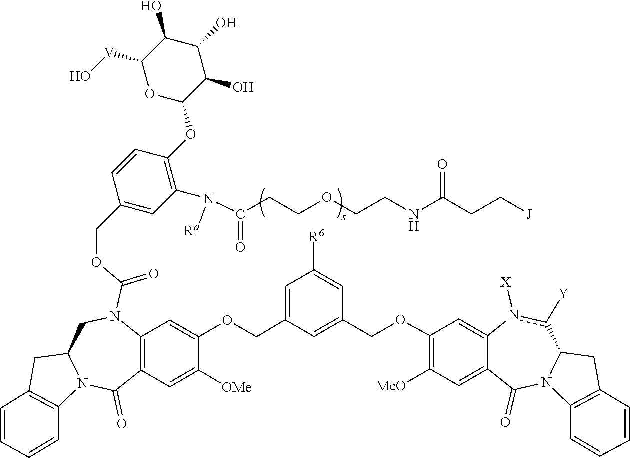 | (IIIb4C) |
| 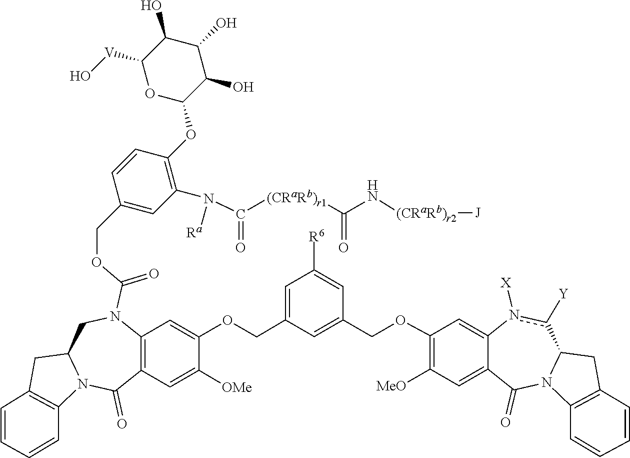 | (IIIb5C) |
| 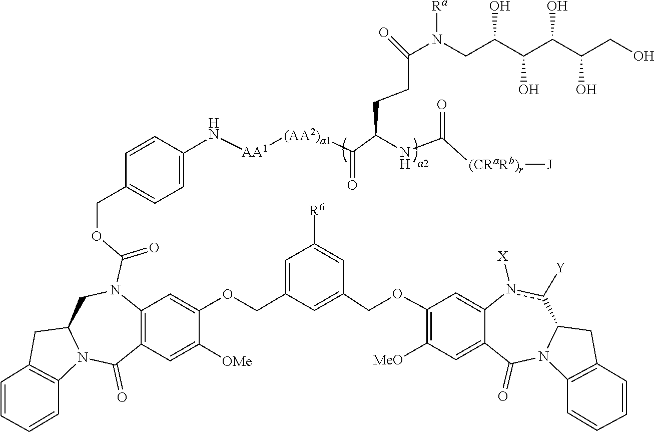 | (IIIc1C) |
| 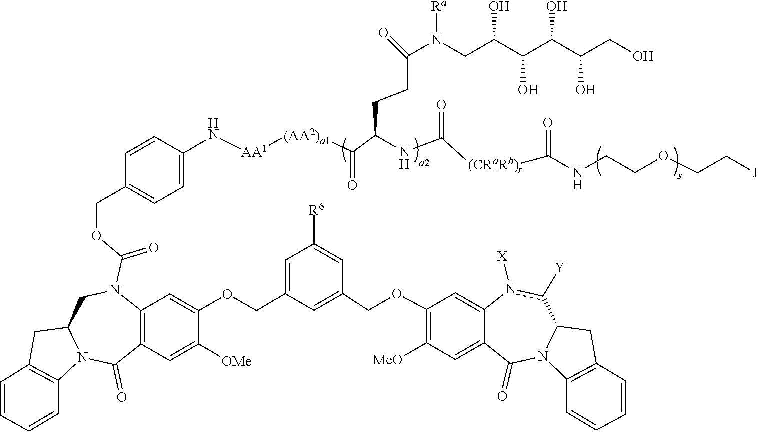 | (IIIc2C) |
| 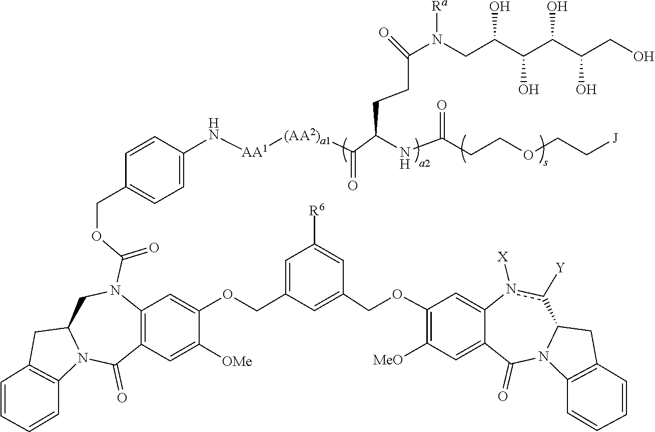 | (IIIc3C) |
| 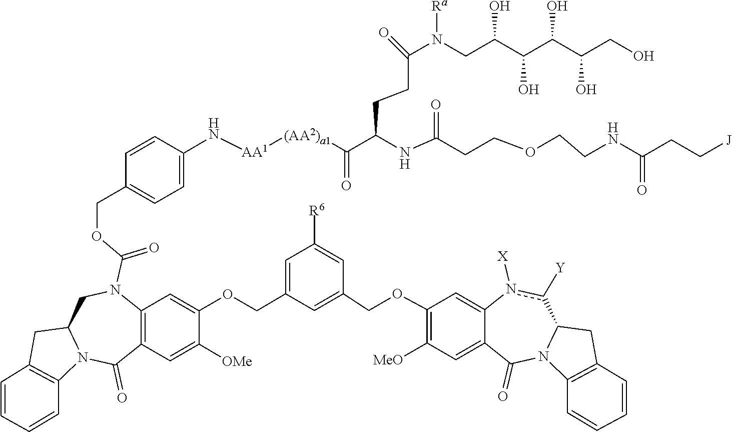 | (IIIc4C) |
| 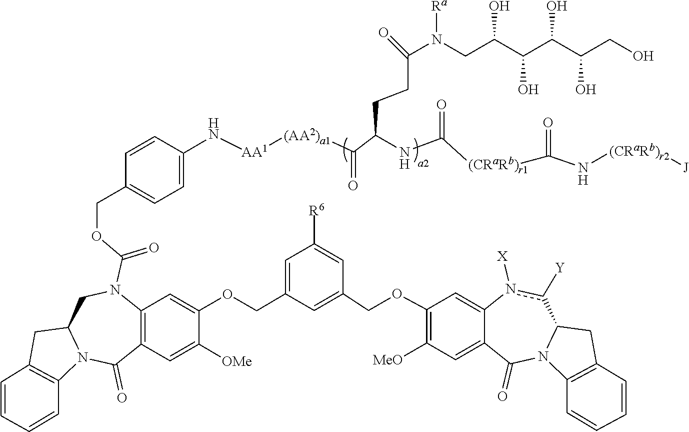 | (IIIc5C) |
| 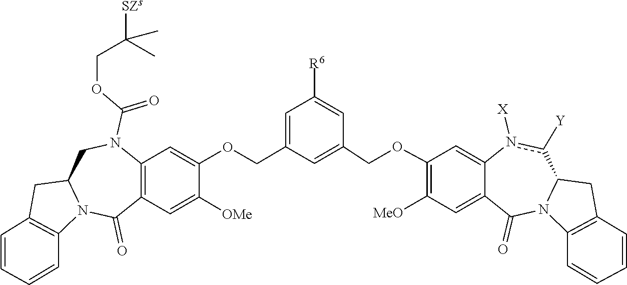 | (IIId1C) |
| 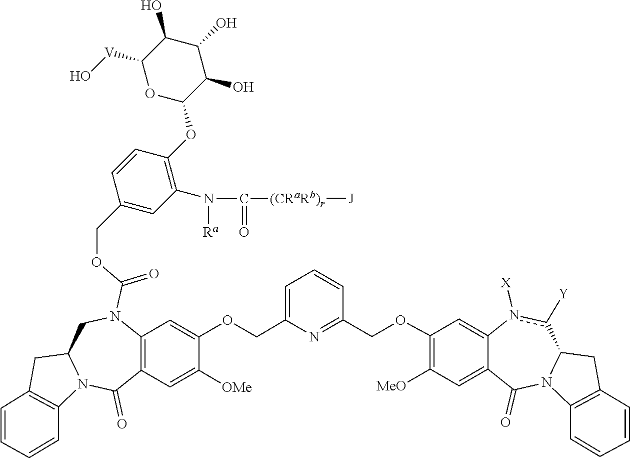 | (IIIb1D) |
| 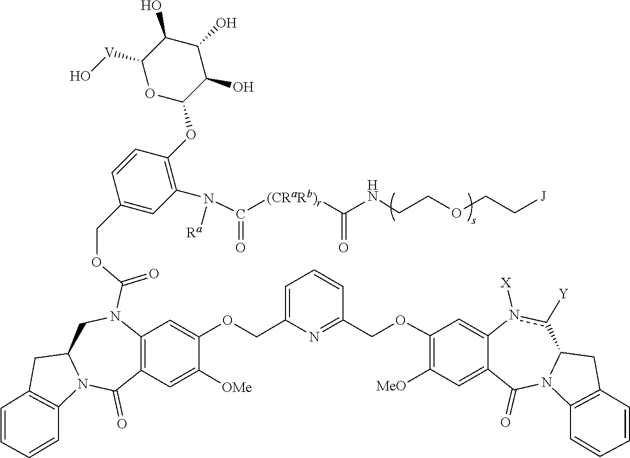 | (IIIb2D) |
| 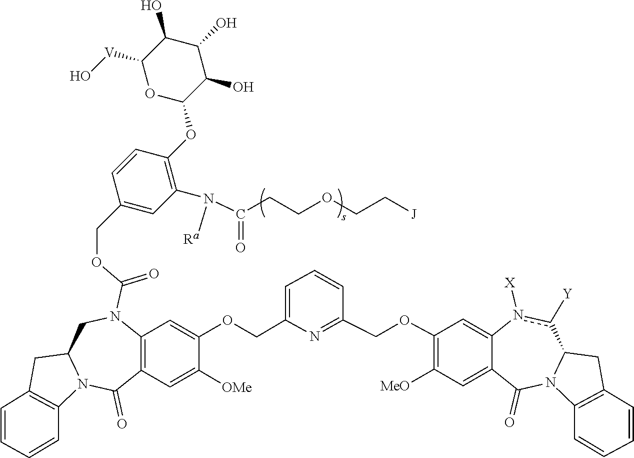 | (IIIb3D) |
| 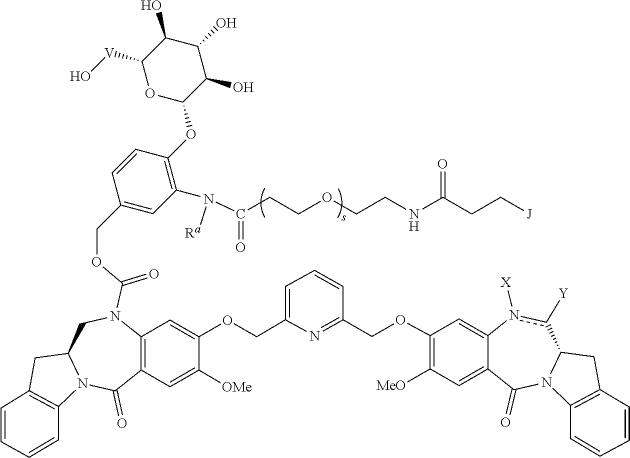 | (IIIb4D) |
| 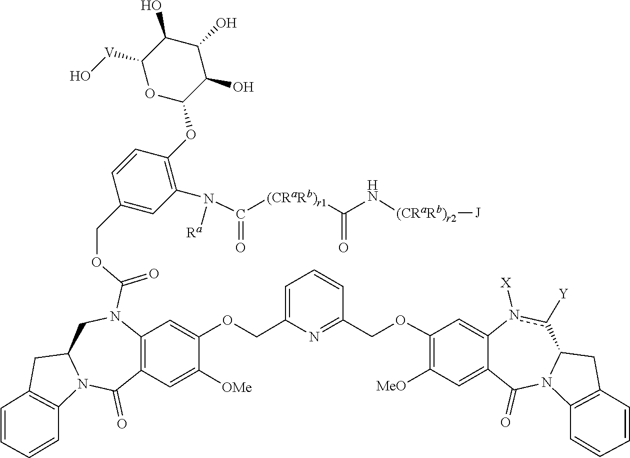 | (IIIb5D) |
| 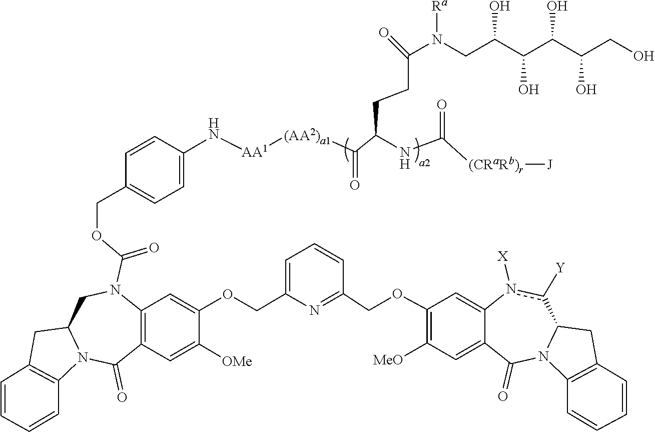 | (IIIc1D) |
| 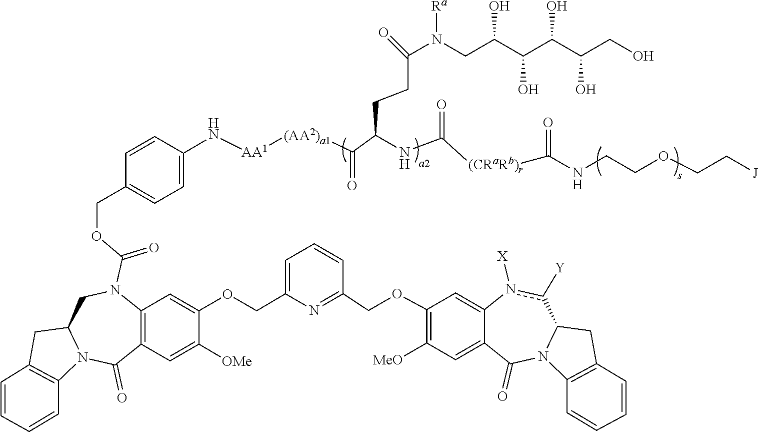 | (IIIc2D) |
| 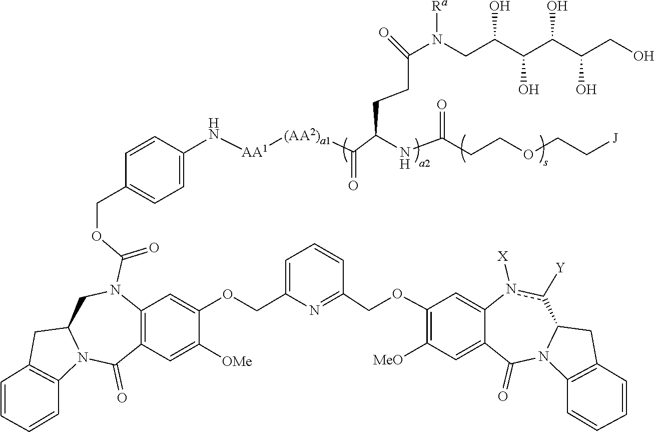 | (IIIc3D) |
| 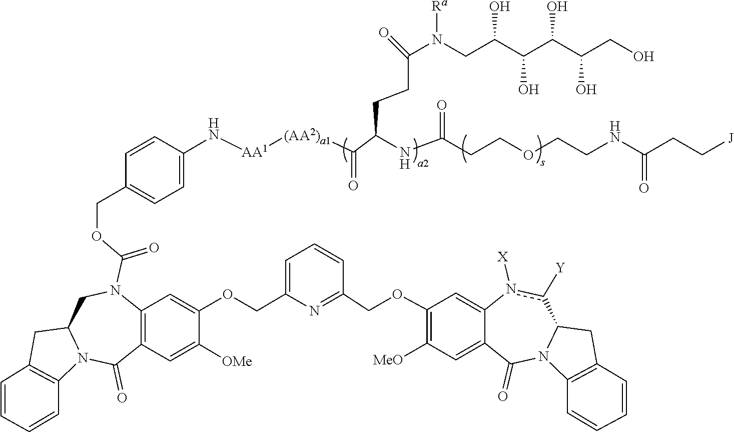 | (IIIc4D) |
| 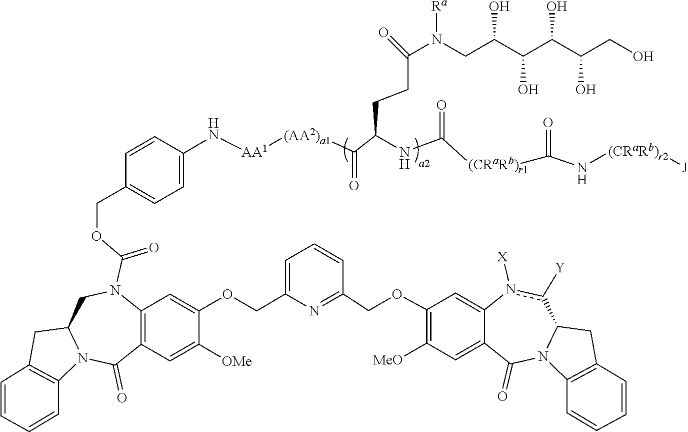 | (IIIc5D) |
| 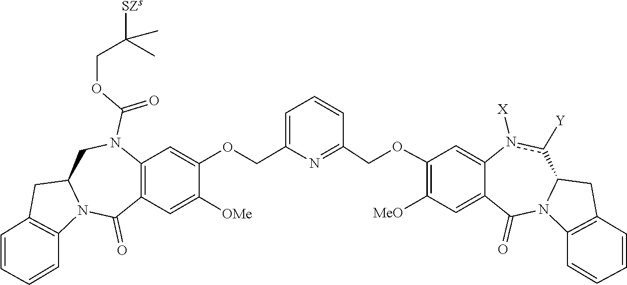 | (IIId1D) |

or a pharmaceutically acceptable salt thereof, wherein:

R⁶ is —C(═O)OR^{6a} or NR^{6b}(CH₂CH₂O)ₙCH₂CH₂OR^{6c};

R^{6a}, R^{6b} and R^{6c} are each independently H or C₁₋₄alkyl;

n is an integer from 1 to 8;

R^{a} and R^{b}, for each occurrence, are independently H or C₁₋₄alkyl;

r, r1 and r2 are each independently an integer from 2 to 6;

s is an integer from 2 to 12; and the remaining variables are as defined in the 6^{th} embodiment.

Also included in the 11^{th} embodiment is a compound represented by the following formula:

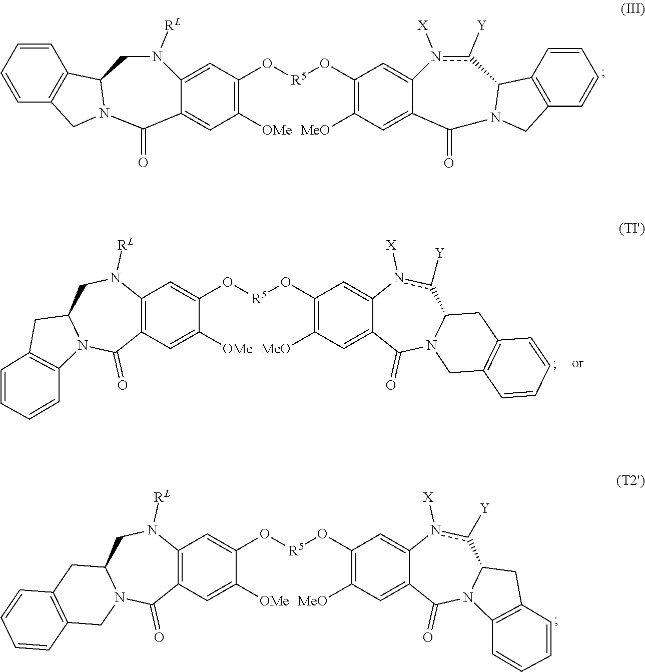

or a pharmaceutically acceptable salt thereof, wherein:

the double line [symbol] between N and C represents a single bond or a double bond, provided that when it is a double bond X is absent and Y is H, or a C₁₋₄alkyl, and when it is a single bond, X is H and Y is —SO₃H;

R^{L} is represented by any one of following formula:

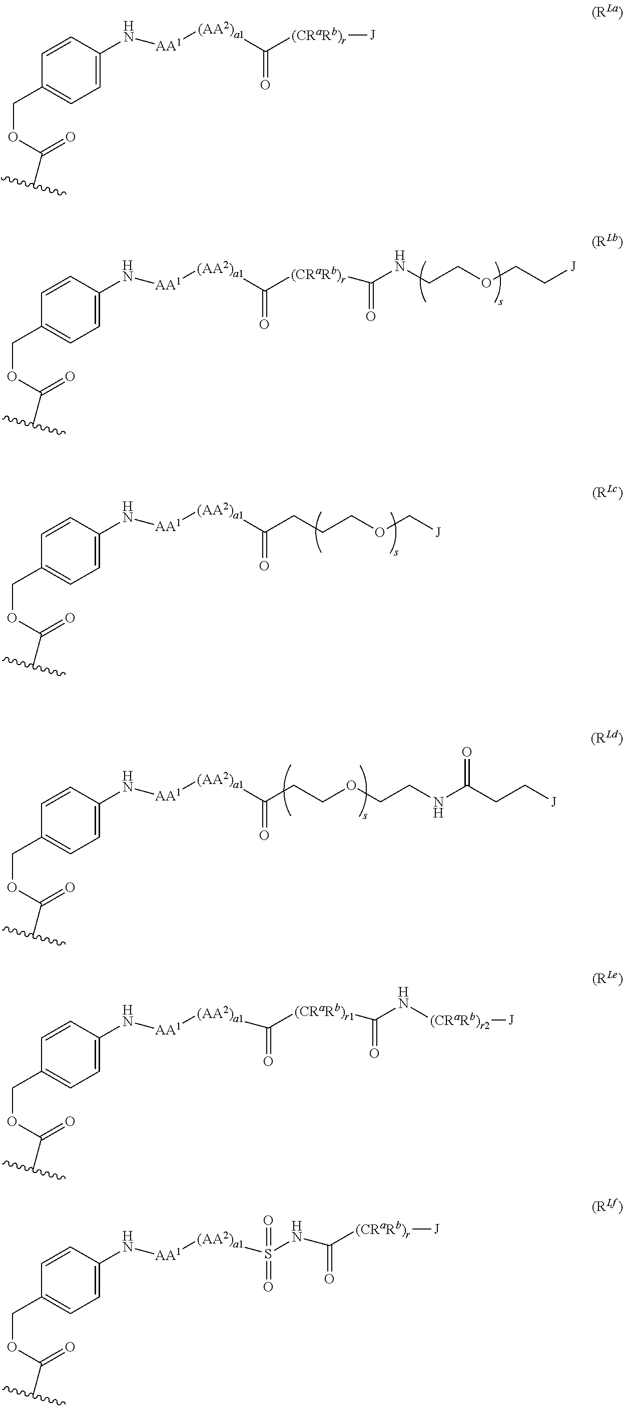

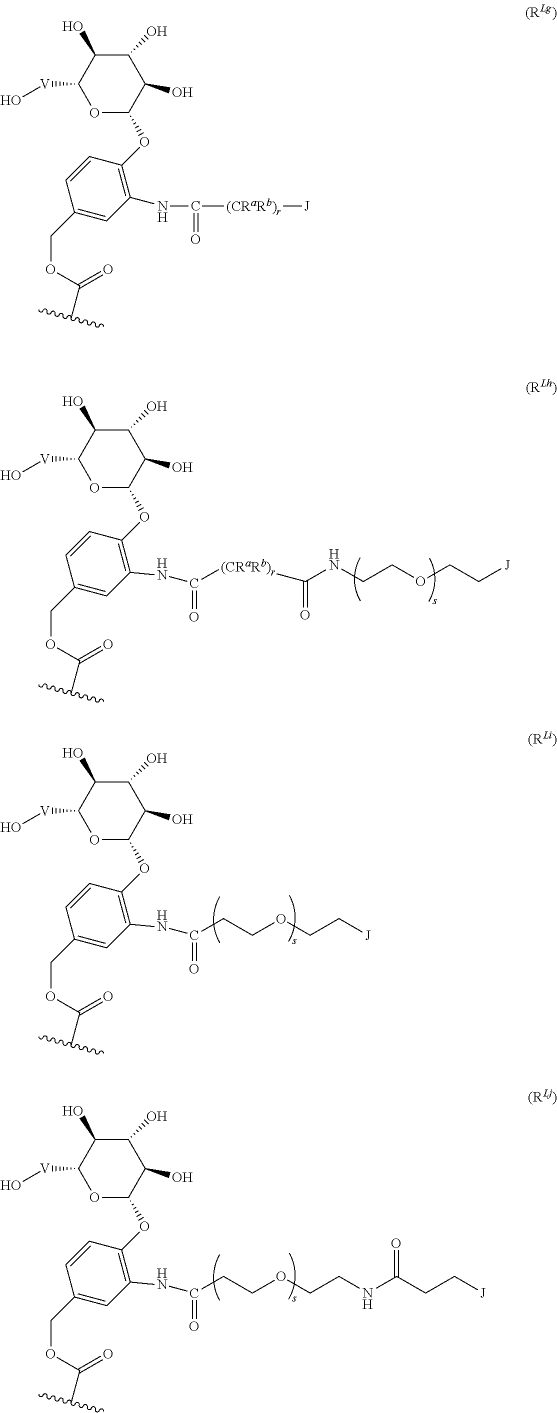

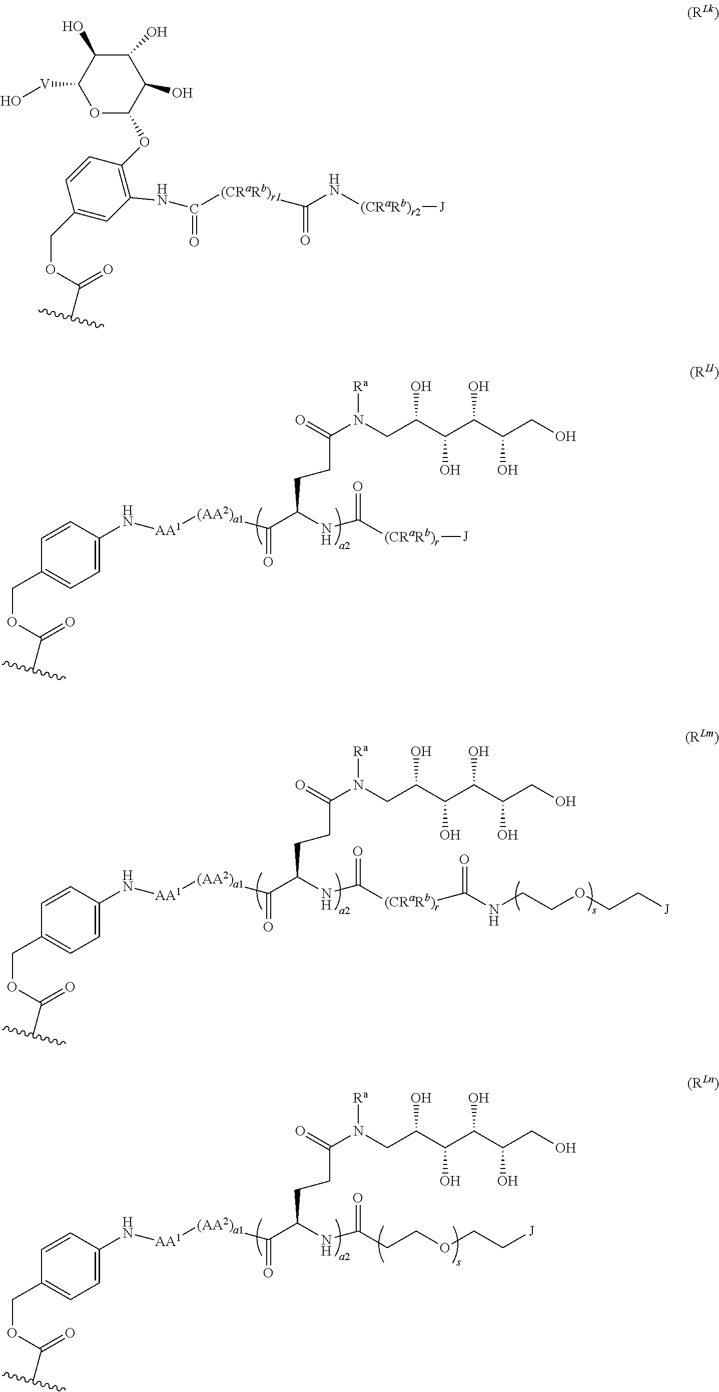

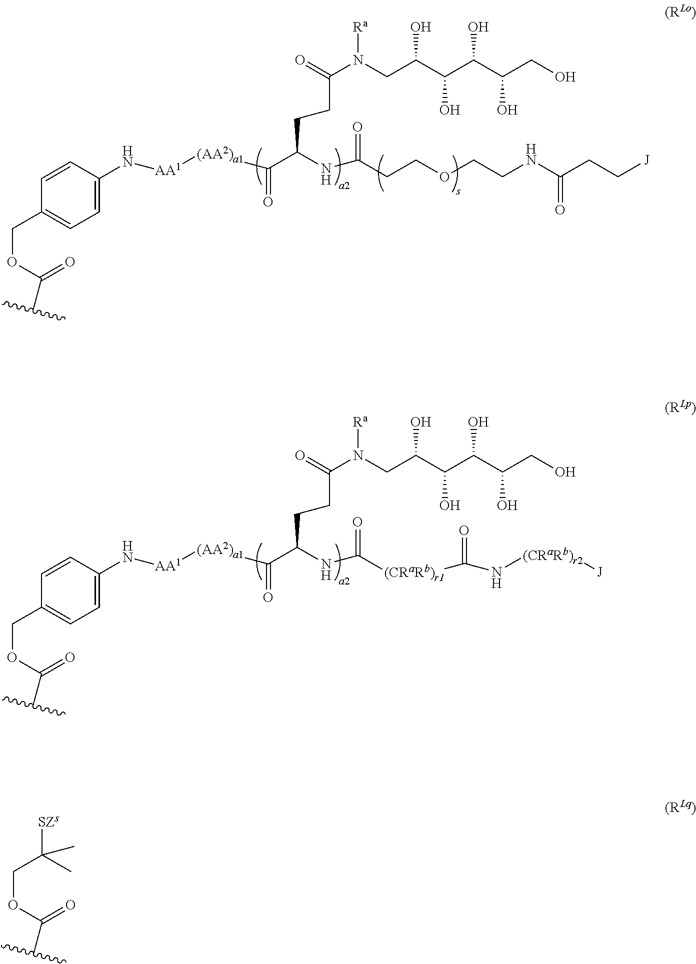

R⁵ is represented by one of the following formulae:

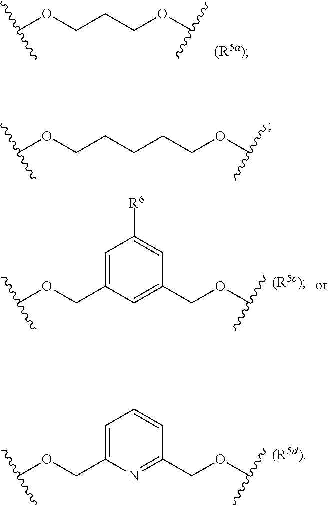

Any combination of R^{L} and R⁵ are included in the invention.

In a 12^{th} embodiment, for compounds described in the 11^{th} embodiment, or a pharmaceutically acceptable salt thereof, the variables defined as:

R^{6a} and R^{6c} are both Me;

R^{6b} is H;

n is 1, 2, 3, or 4;

R^{a} and R^{b}, for each occurrence, are independently H or Me;

r is 4;

r1 is 4;

r2 is 2;

s is 1, 2, 3 or 4; and the remaining variables are as defined in the 11^{th} embodiment.

In a 13^{th} embodiment, for compounds described in the 2^{nd} to 12^{th} embodiments, or a pharmaceutically acceptable salt thereof, J is —COOR^{d} or a reactive ester represented by COE, wherein R^{d} is H or a C₁₋₄alkyl; and the remaining variables are as defined in the 2^{nd}, 3^{rd}, 4^{th}, 5^{th}, 6^{th}, 7^{th}, 8^{th}, 9^{th}, 10^{th}, 11^{th}, or 12^{th} embodiment or any specific embodiments described therein.

In a specific embodiment, J is a reactive ester selected from N-hydroxysuccinimide ester, N-hydroxy sulfosuccinimide ester, nitrophenyl (e.g., 2 or 4-nitrophenyl) ester, dinitrophenyl (e.g., 2,4-dinitrophenyl) ester, sulfo-tetraflurophenyl (e.g., 4-sulfo-2,3,5,6-tetrafluorophenyl) ester, and pentafluorophenyl ester.

In a more specific embodiment, J is N-hydroxysuccinimide ester.

In a 14^{th} embodiment, for compounds described in any one of the 2^{nd} to 12^{th} embodiments, or a pharmaceutically acceptable salt thereof, J is

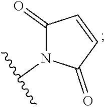 [SMILES: *N1C(=O)C=CC1=O]

and the remaining variables are as defined in the 2^{nd}, 3^{rd}, 4^{th}, 5^{th}, 6^{th}, 7^{th}, 8^{th}, 9^{th}, 10^{th}, 11^{th}, or 12^{th} embodiment or any specific embodiment described therein.

In a 15^{th} embodiment, for compounds described in any one of the 2^{nd} to 12^{th} embodiments, or a pharmaceutically acceptable salt thereof, J is —SZ^{s}; Z^{s} is H, SR^{e}, or is selected from the following formulae:

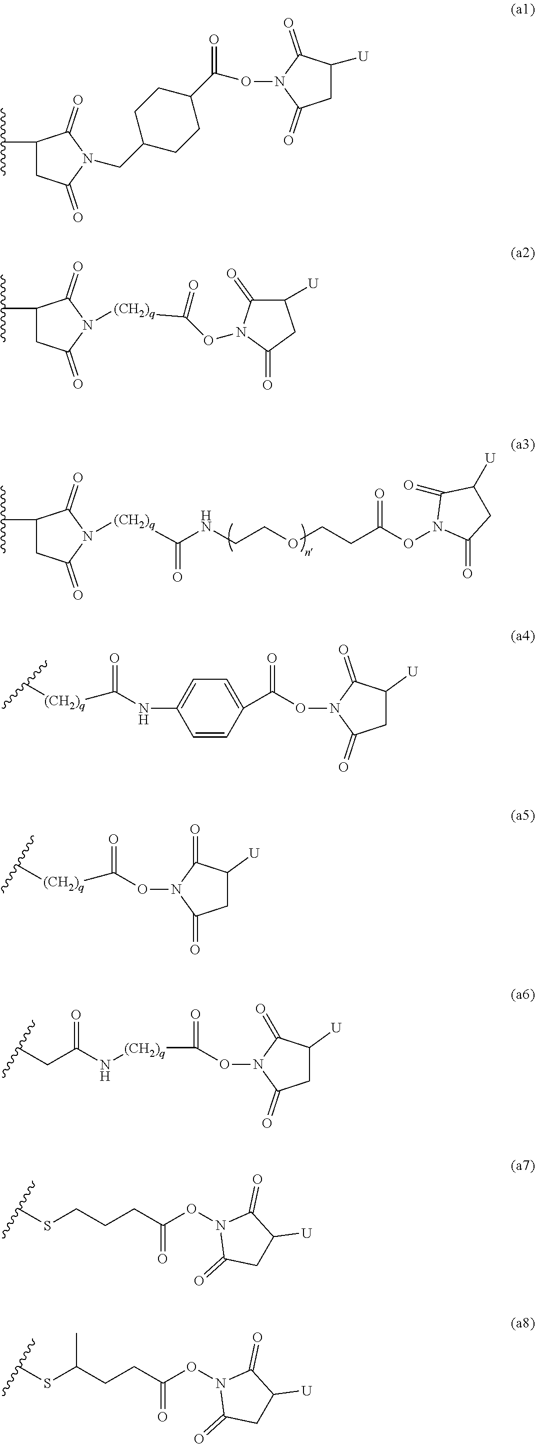

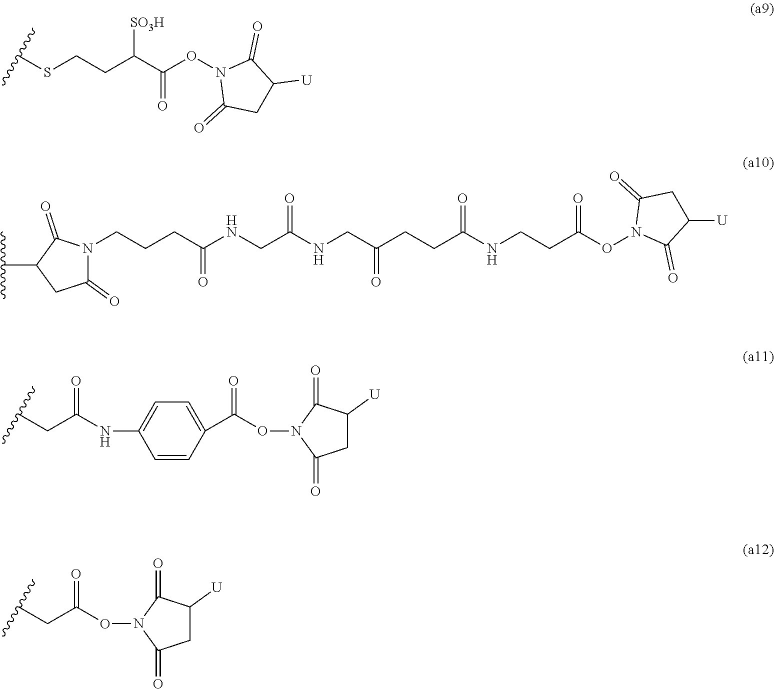

wherein:

q is an integer from 1 to 5;

n′ is an integer from 2 to 6;

U is —H or SO₃H;

R^{e} is a linear or branched alkyl having 1 to 6 carbon atoms or is selected from phenyl, nitrophenyl (e.g., 2 or 4-nitrophenyl), dinitrophenyl (e.g., 2,4-dinitrophenyl), carboxynitrophenyl (e.g., 3-carboxy-4-nitrophenyl), pyridyl or nitropyridyl (e.g., 4-nitropyridyl); and

the remaining variables are as defined in the 2^{nd}, 3^{rd}, 4^{th}, 5^{th}, 6^{th}, 7^{th}, 8^{th}, 9^{th}, 10^{th}, 11^{th}, or 12^{th} embodiment or any specific embodiment described therein.

In a specific embodiment, Z^{s} is H. In another specific embodiment, Z^{s} is —SR^{e}, wherein R^{e} is methyl. In yet another specific embodiment, Z^{s} is represented by formula (a7) or (a9). In another specific embodiment, Z^{s} is represented by formula (a16) or (a17).

In a 16^{th} embodiment, for compounds described in any one of the 1^{st} to 15^{th} embodiments, or a pharmaceutically acceptable salt thereof, the double line [symbol] between N and C represents a double bond, X is absent and Y is H; and the remaining variables are as defined in the 1^{st} 2^{nd} 3^{rd}, 4^{th}, 5^{th}, 6^{th}, 7^{th}, 8^{th}, 9^{th}, 10^{th}, 11^{th}, 12^{th}, 13^{th}, 14^{th}, or 15^{th} embodiment or any specific embodiments described therein.

In a 17^{th} embodiment, for compounds described in any one of the 1^{st} to 15^{th} embodiments, or a pharmaceutically acceptable salt thereof, the double line [symbol] between N and C represents a single bond, X is H and Y is —SO₃H; and the remaining variables are as defined in the 1^{st}, 2^{nd} 3^{rd}, 4^{th}, 5^{th}, 6^{th}, 7^{th}, 8^{th}, 9^{th}, 10^{th}, 11^{th}, 12^{th}, 13^{th}, 14^{th}, or 15^{th} embodiment or any specific embodiments described therein. In a specific embodiment, the pharmaceutically acceptable salt is a sodium or potassium salt. In another specific embodiment, the pharmaceutically acceptable salt is a sodium salt.

In a 18^{th} embodiment, for compounds described in any one the 2^{nd} to 17^{th} embodiments, or a pharmaceutically acceptable salt thereof, a1 is an integer from 1 to 7; and the remaining variables are as defined in the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, or 17^{th} embodiment or any specific embodiments described therein.

In a specific embodiment, AA′ and AA² are each independently selected from Arginine (Arg), Histidine (His), Lysine (Lys), Aspartic acid (Asp), Glutamic Acid (Glu), Serine (Ser), Threonine (Thr), Asparagine (Asn), Glutamine (Gln), Cysteine (Cys), Selenocysteine (Sec), Glycine (Gly), Proline (Pro), Alanine (Ala), Valine (Val), Isoleucine (Ile), Leucine (Leu), Methionine (Met), Phenylalanine (Phe), Tyrosine (Tyr) and Tryptophan (Trp).

In a 19^{th} embodiment, for compounds described in the 18^{th} embodiment, or a pharmaceutically acceptable salt thereof, AA¹-(AA²)ₐ₁ is selected from Gly-Gly-Gly, Ala-Val, Val-Ala, Val-Cit, Val-Lys, Phe-Lys, Lys-Lys, Ala-Lys, Phe-Cit, Leu-Cit, Lle-Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu, β-Ala-Leu-Ala-Leu, Gly-Phe-Leu-Gly, Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Thr-Thr, Thr-Met, Met-Thr, Leu-Ala, Cit-Val, Gln-Val, Ser-Val, Leu-Gln, Gln-Leu, Phe-Arg, Arg-Phe, Tyr-Arg, Arg-Tyr, Phe-Gln, Gln-Phe, Val-Thr, Thr-Val, Met-Tyr, and Tyr-Met; and the remaining variables are as defined in the 18^{th} embodiment.

In a specific embodiment, AA¹-(AA²)ₐ₁ is Ala-Ala, L-Ala-L-Ala, Ala-Val, L-Ala-L-Val, Gln-Val, L-Gln-L-Val, Gln-Leu, L-Gln-L-Leu, Ser-Val, or L-Ser-L-Val.

In a 20^{th} embodiment, the compound of the present invention is represented by the following formula in Table D:

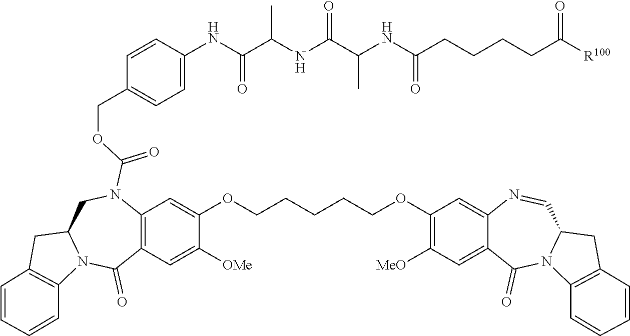

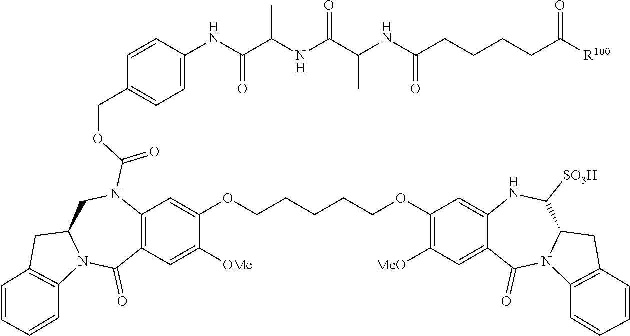

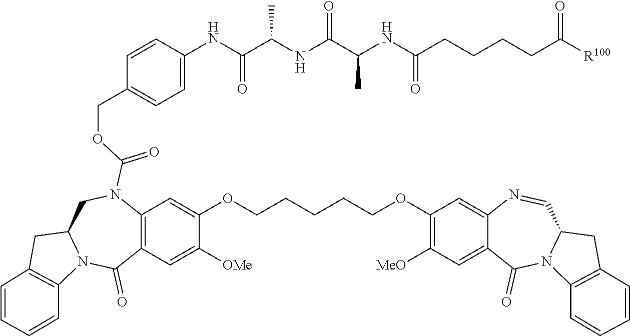

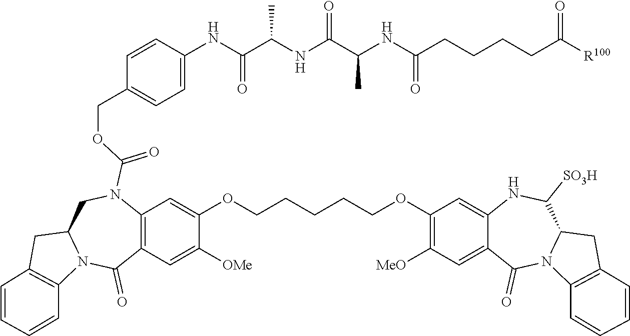

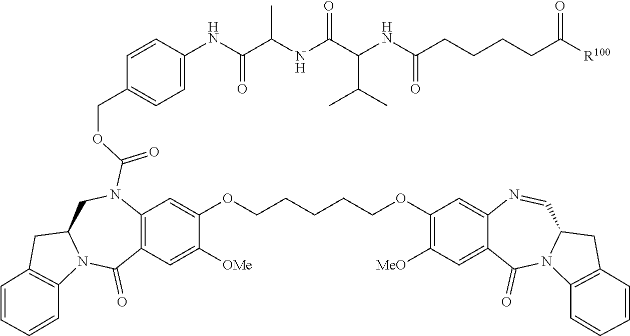

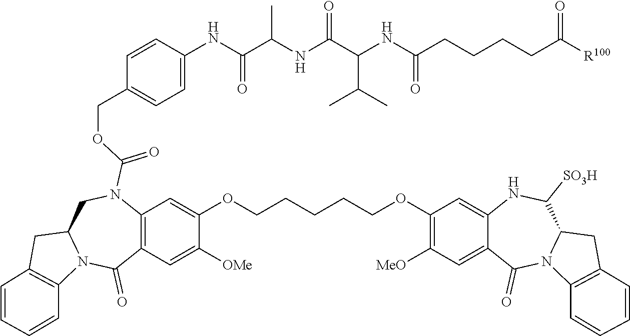

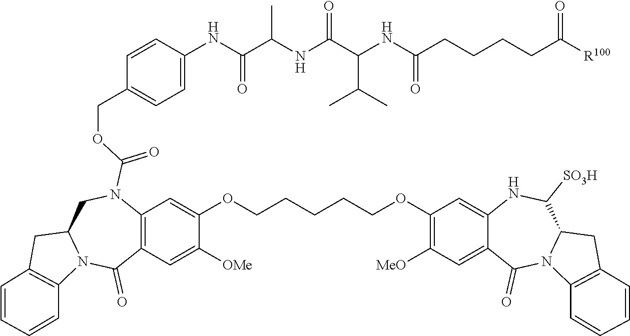

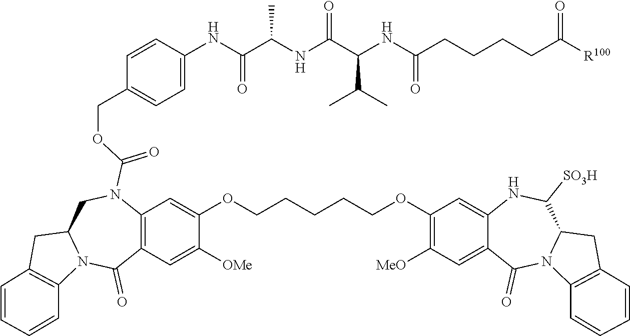

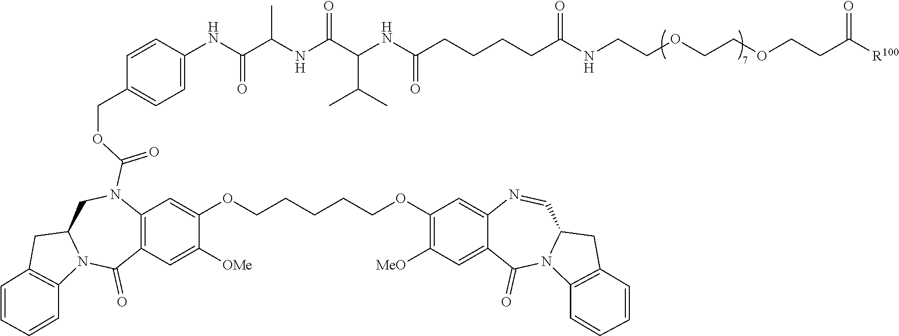

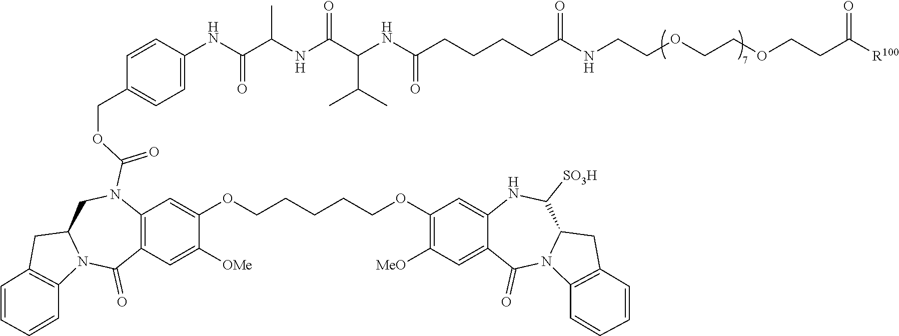

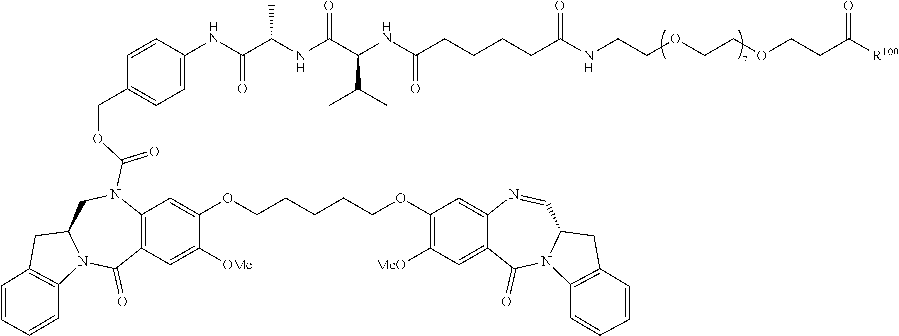

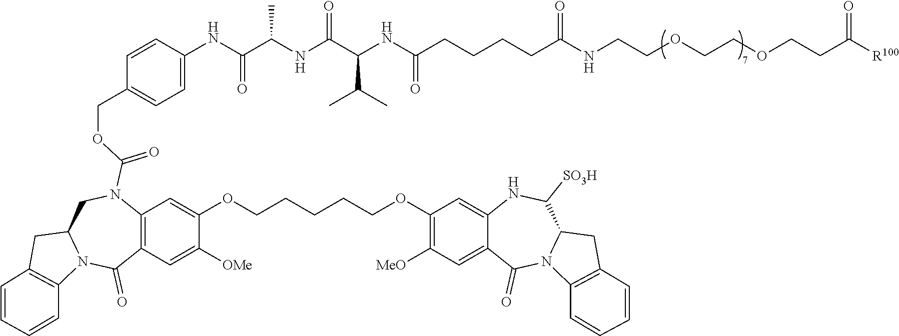

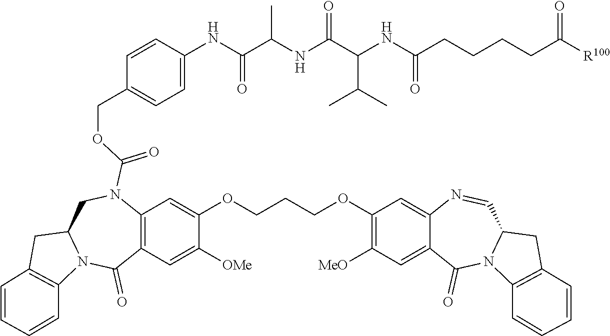

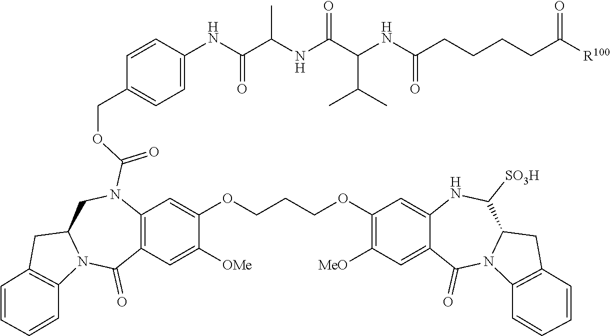

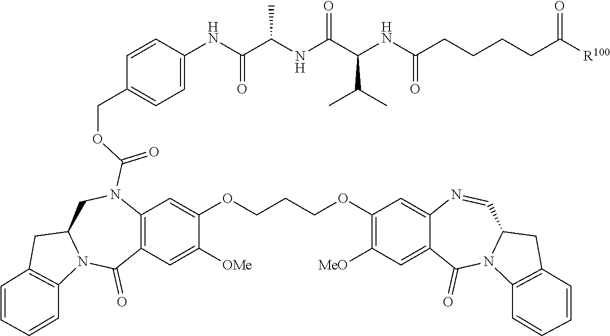

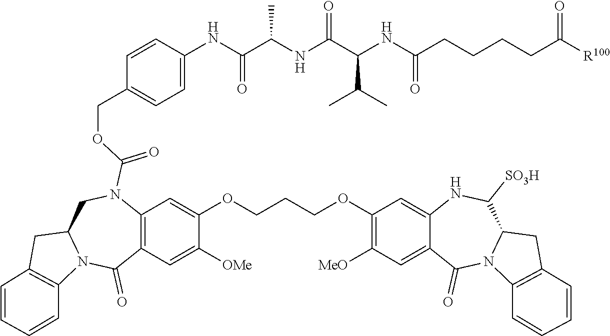

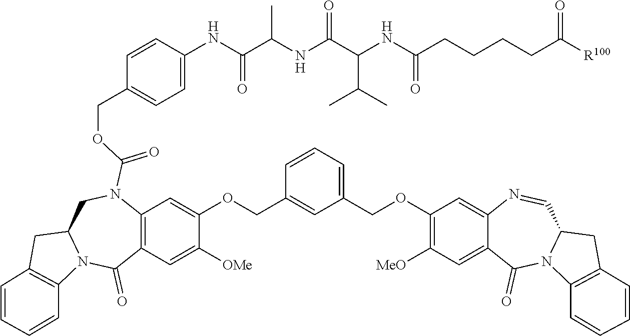

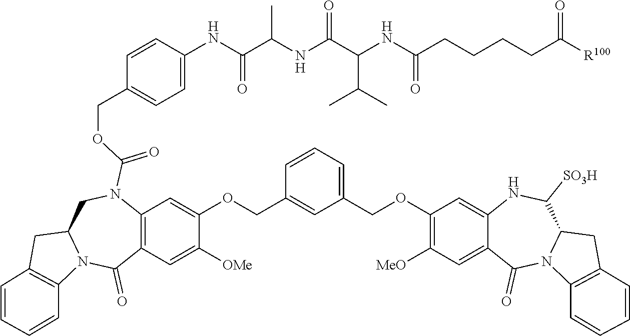

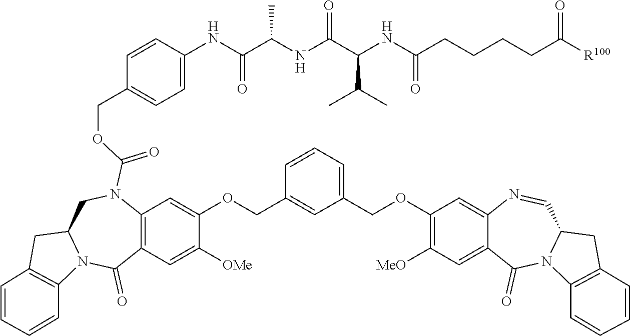

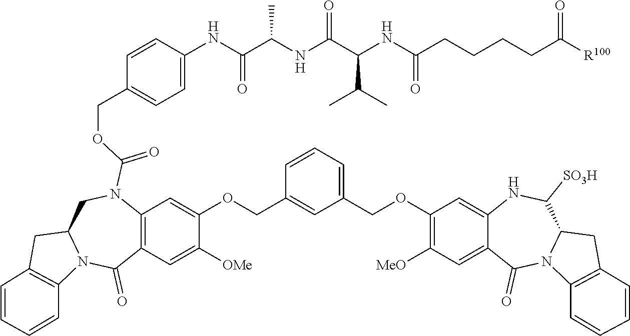

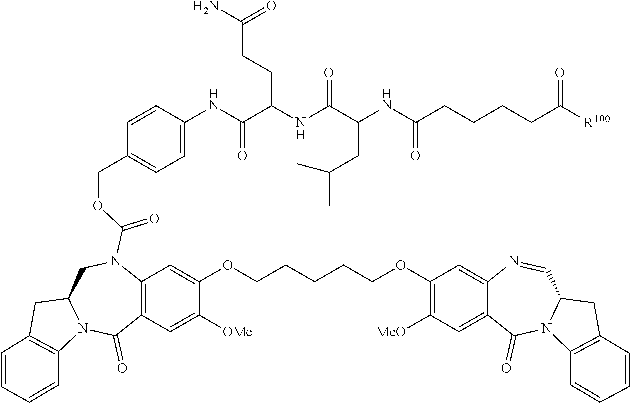

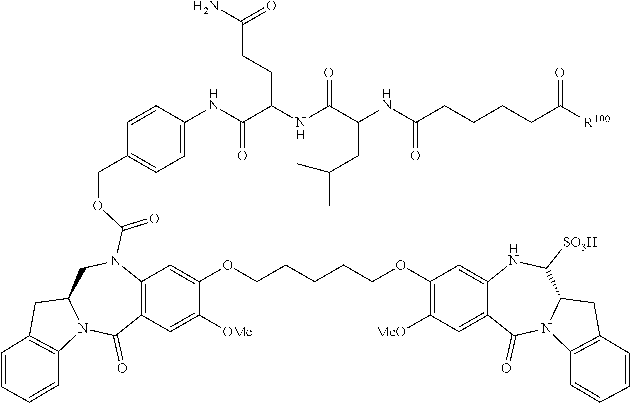

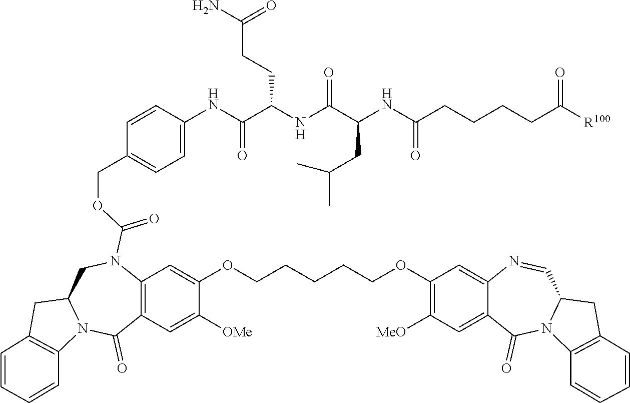

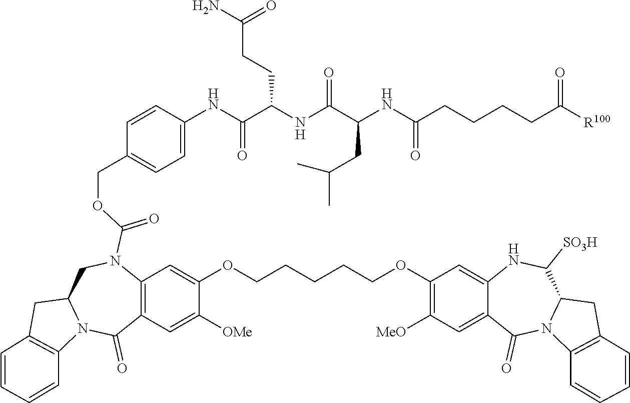

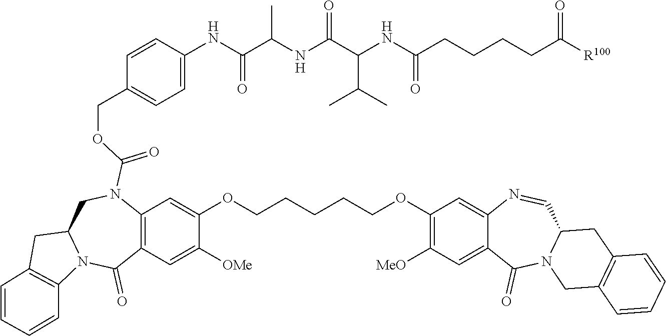

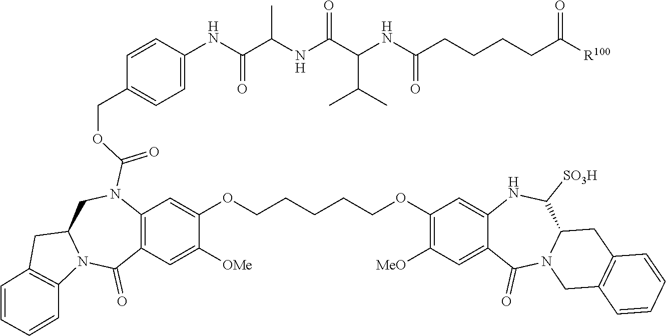

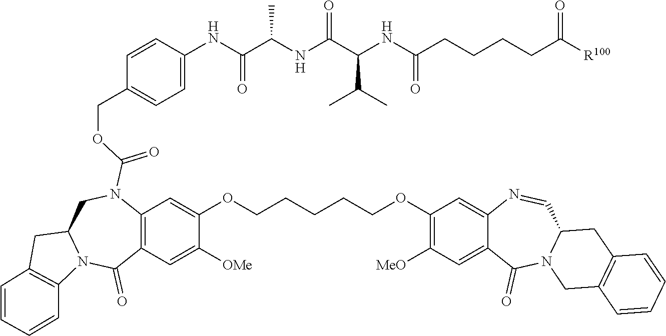

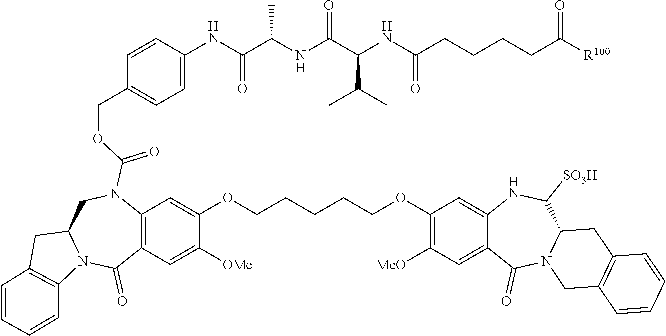

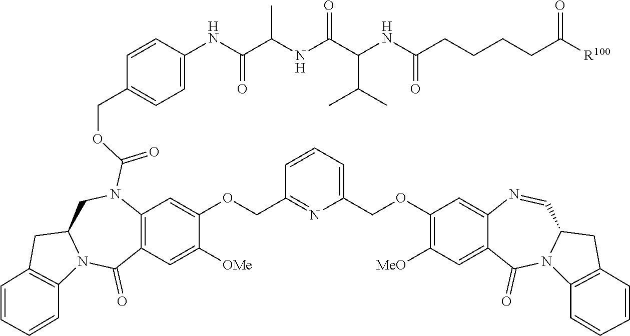

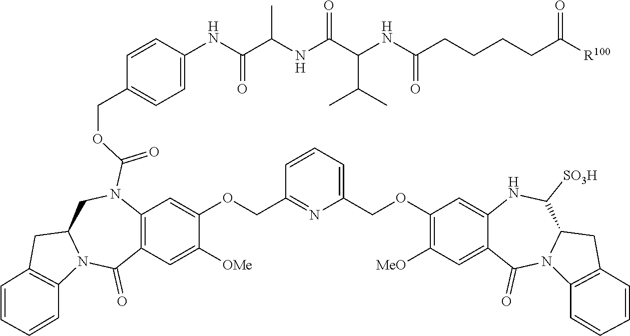

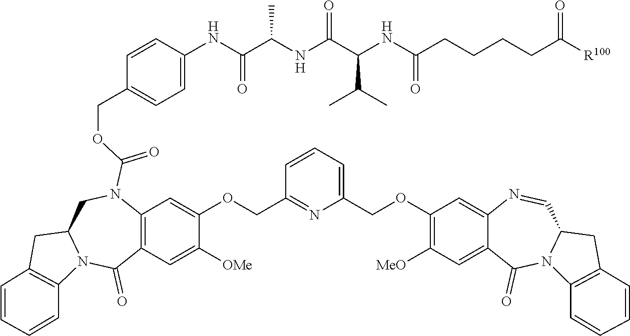

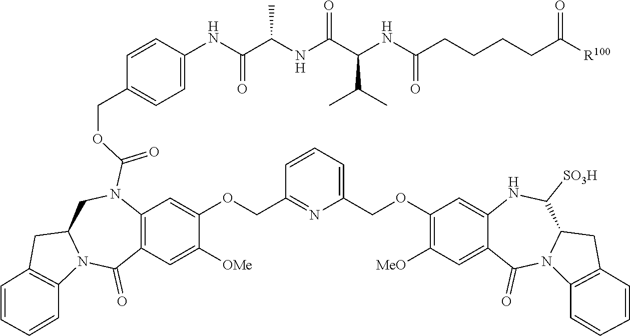

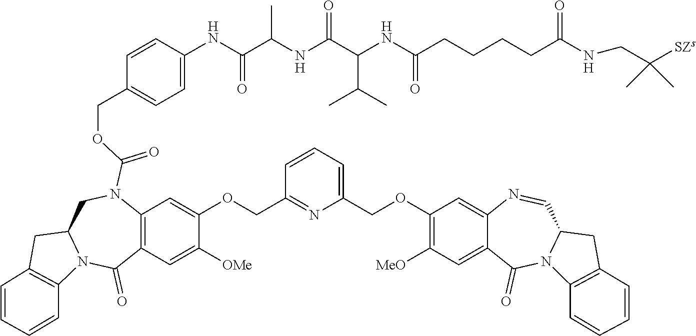

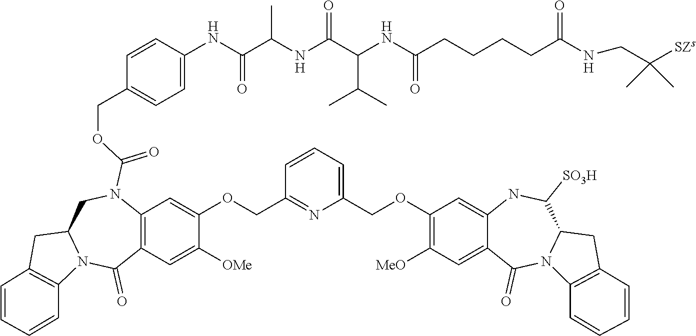

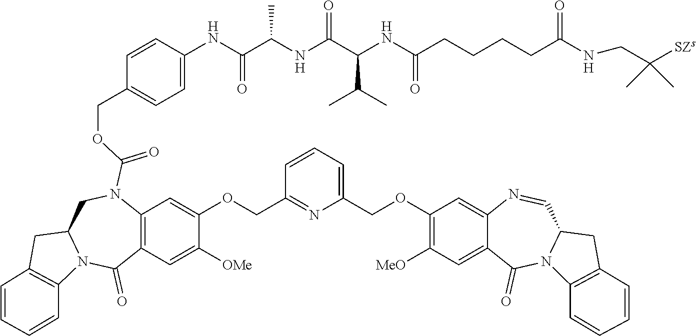

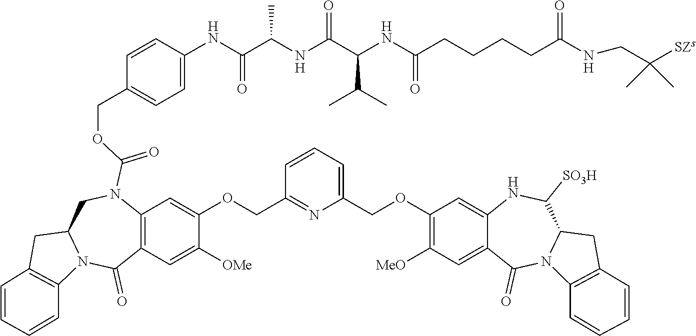

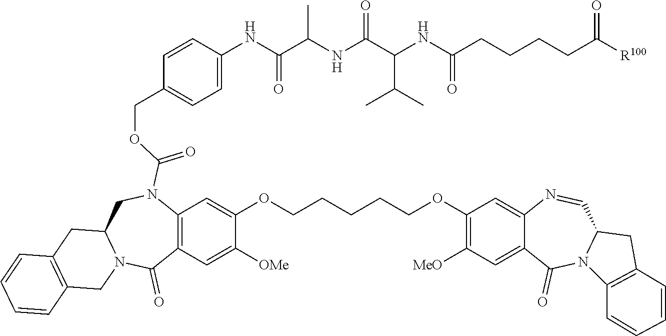

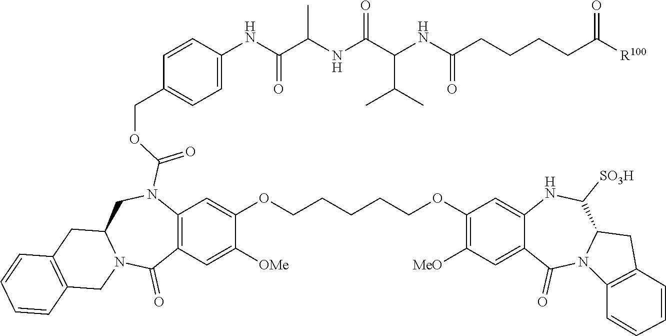

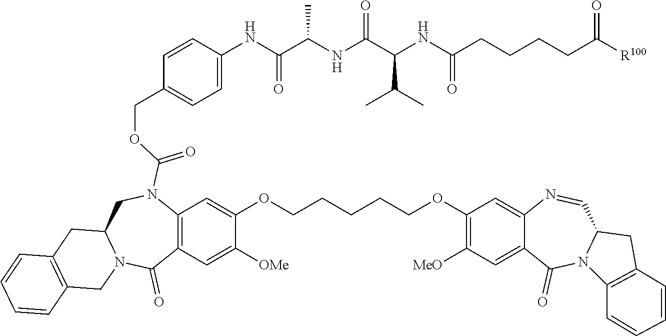

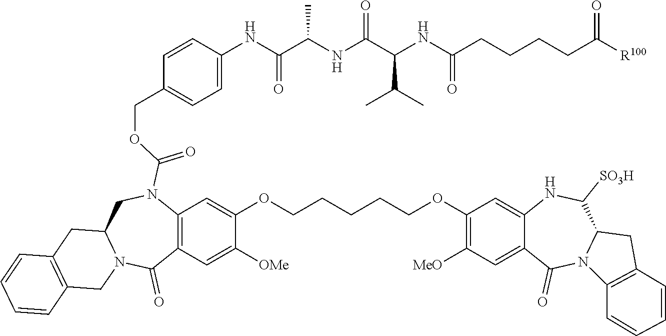

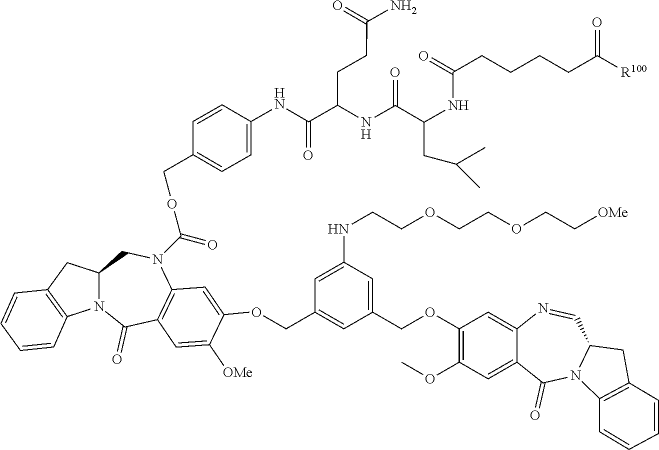

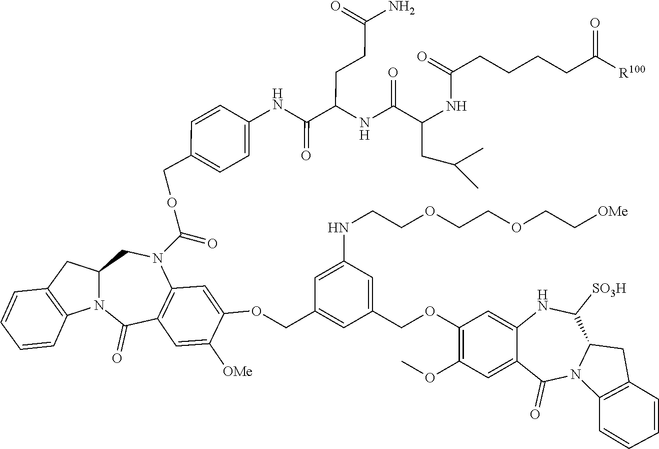

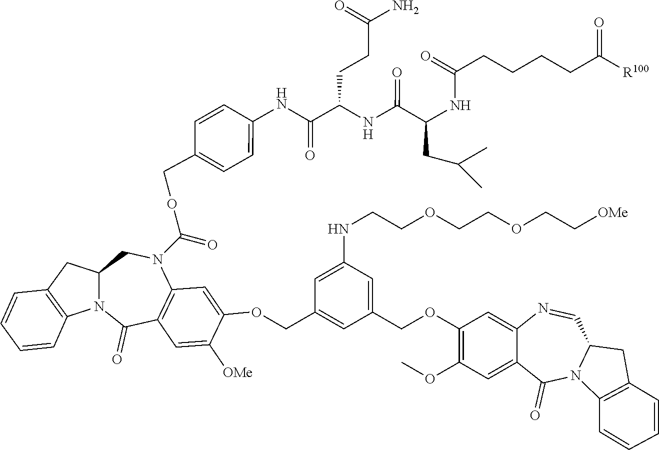

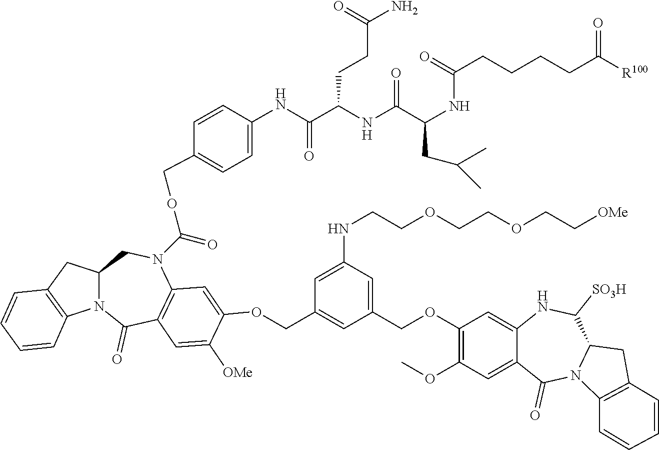

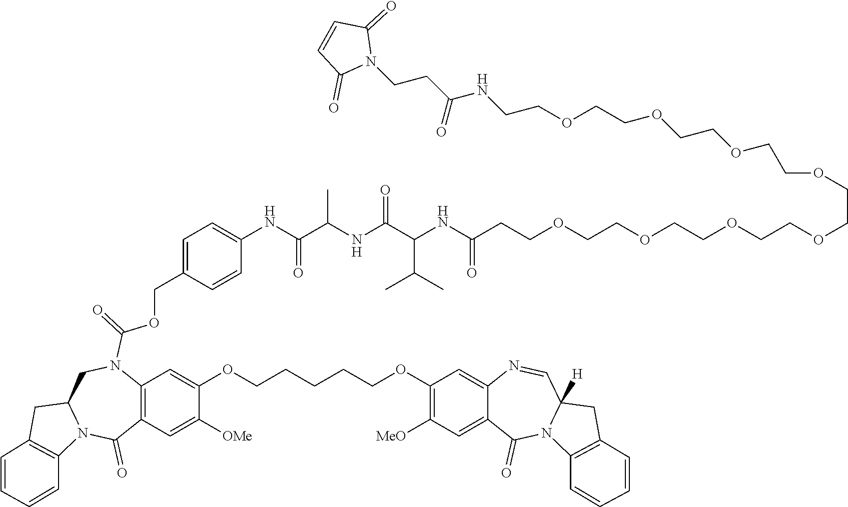 [SMILES: COc1cc2c(cc1OCCCCCOc1cc3c(cc1OC)C(=O)N1c4ccccc4C[C@H]1CN3C(=O)OCc1ccc(NC(=O)C(C)NC(=O)C(NC(=O)CCOCCOCCOCCOCCOCCOCCOCCOCCNC(=O)CCN3C(=O)C=CC3=O)C(C)C)cc1)N=C[C@@H]1Cc3ccccc3N1C2=O]

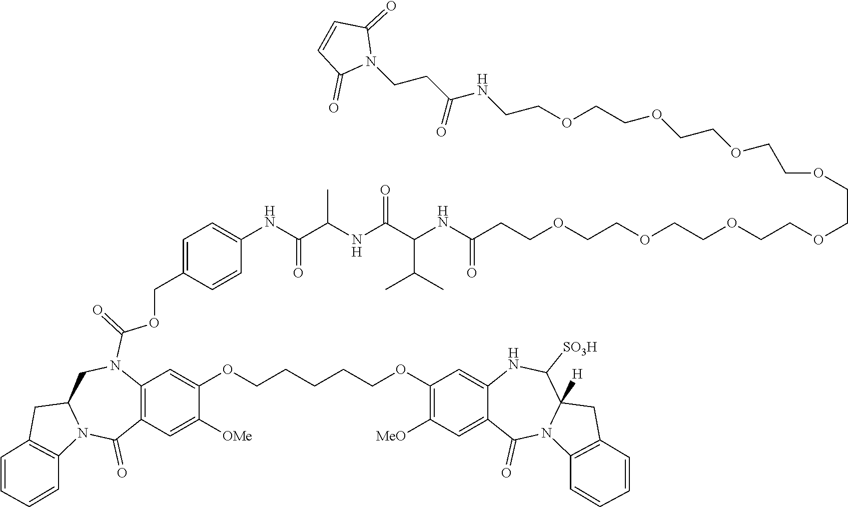 [SMILES: COc1cc2c(cc1OCCCCCOc1cc3c(cc1OC)C(=O)N1c4ccccc4C[C@H]1CN3C(=O)OCc1ccc(NC(=O)C(C)NC(=O)C(NC(=O)CCOCCOCCOCCOCCOCCOCCOCCOCCNC(=O)CCN3C(=O)C=CC3=O)C(C)C)cc1)NC(S(=O)(=O)O)[C@@H]1Cc3ccccc3N1C2=O]

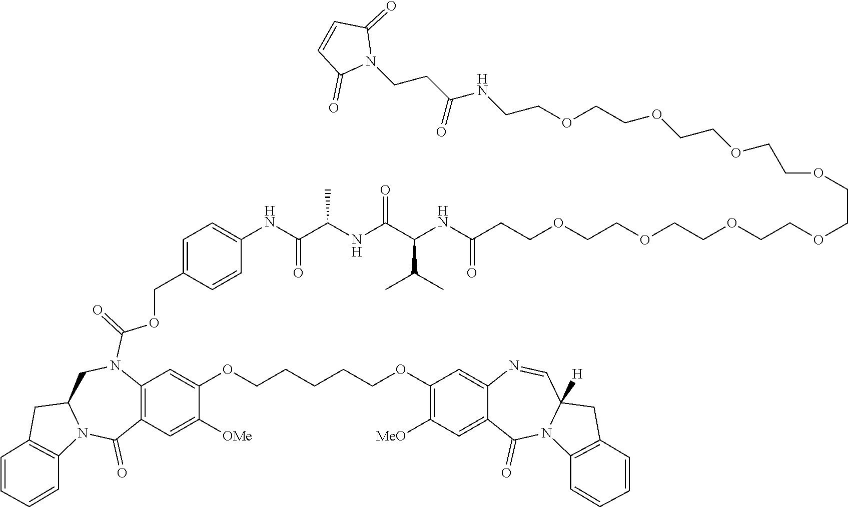 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)cc1)N=C[C@@H]1Cc3ccccc3N1C2=O]

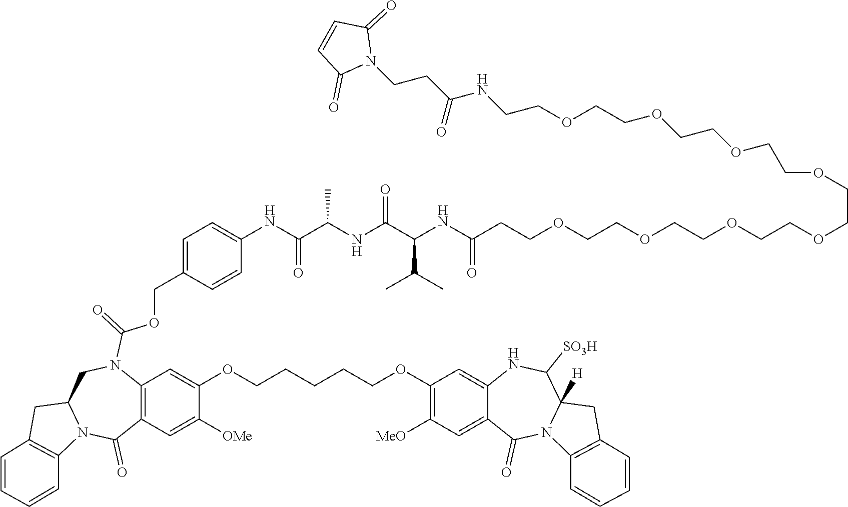 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)cc1)NC(S(=O)(=O)O)[C@@H]1Cc3ccccc3N1C2=O]

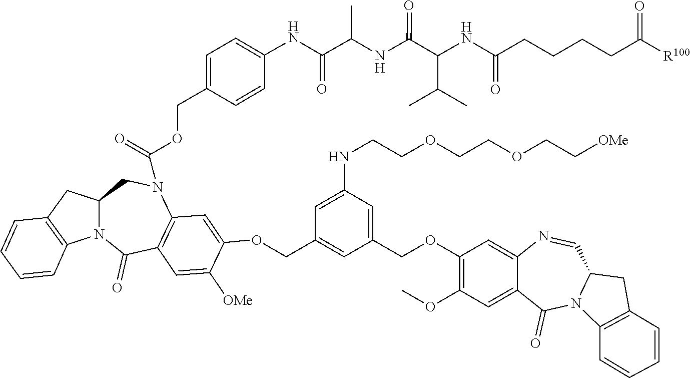

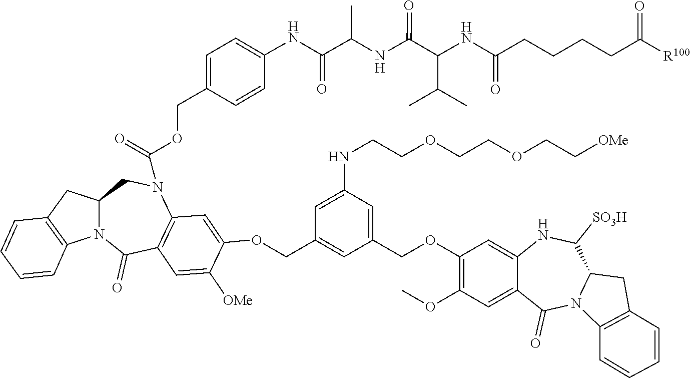

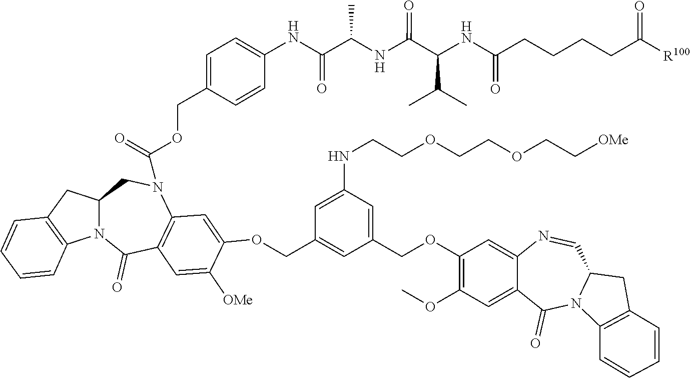

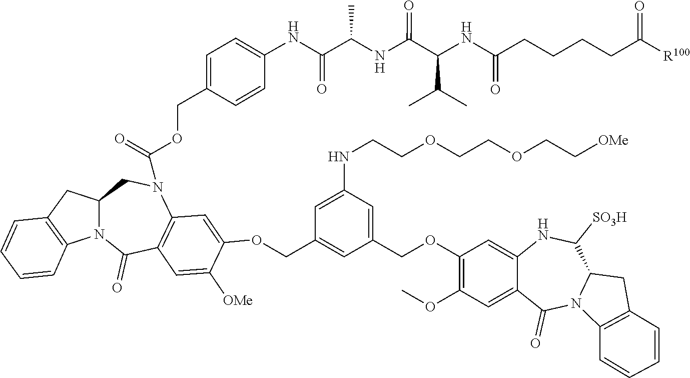

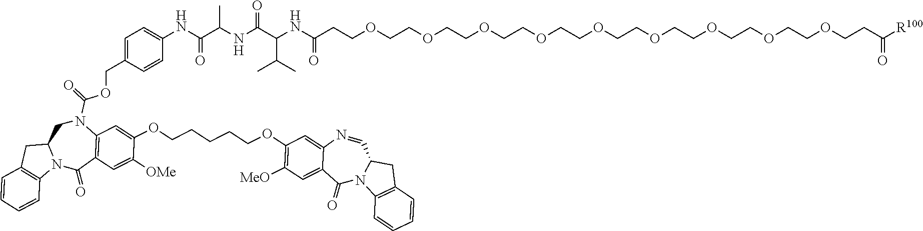

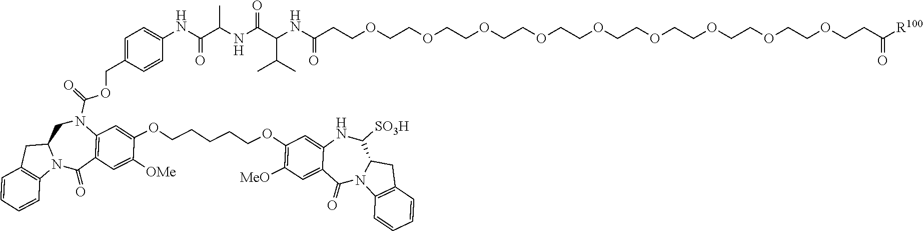

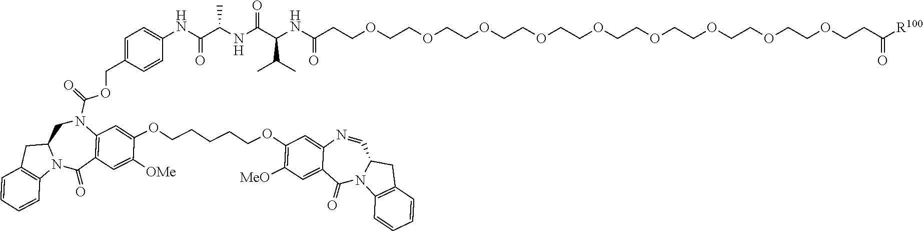

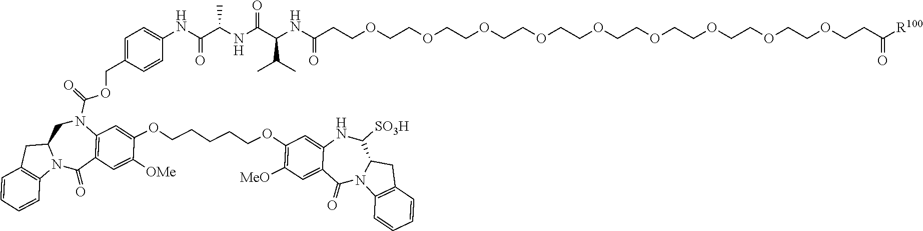

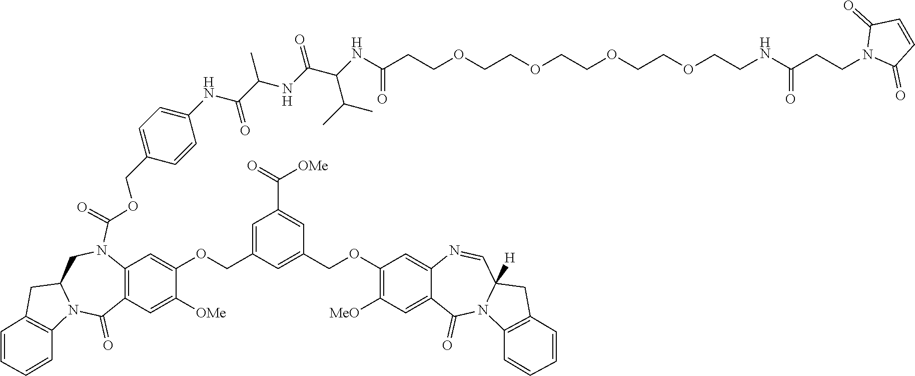 [SMILES: COC(=O)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)C(C)NC(=O)C(NC(=O)CCOCCOCCOCCOCCNC(=O)CCN3C(=O)C=CC3=O)C(C)C)cc2)c1]

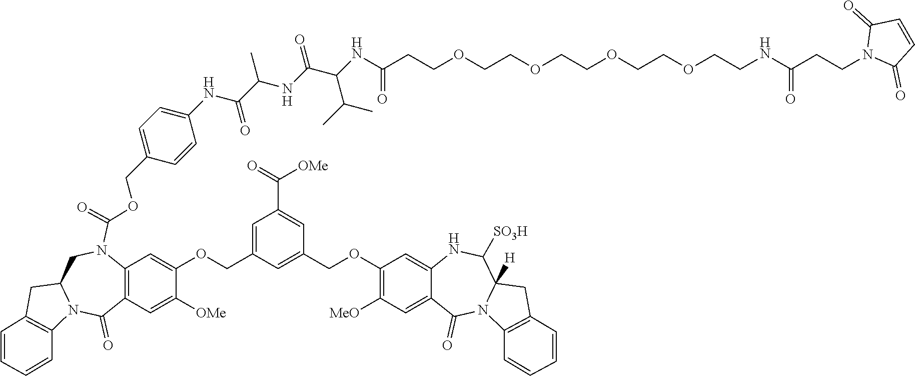 [SMILES: COC(=O)c1cc(COc2cc3c(cc2OC)C(=O)N2c4ccccc4C[C@H]2C(S(=O)(=O)O)N3)cc(COc2cc3c(cc2OC)C(=O)N2c4ccccc4C[C@H]2CN3C(=O)OCc2ccc(NC(=O)C(C)NC(=O)C(NC(=O)CCOCCOCCOCCOCCNC(=O)CCN3C(=O)C=CC3=O)C(C)C)cc2)c1]

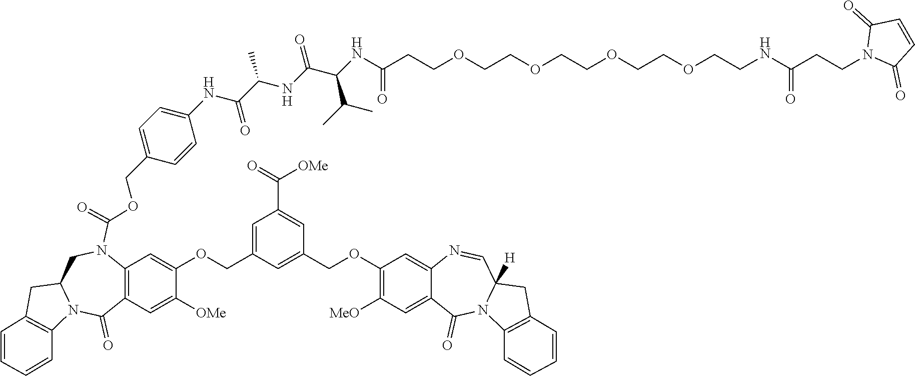 [SMILES: COC(=O)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](NC(=O)CCOCCOCCOCCOCCNC(=O)CCN3C(=O)C=CC3=O)C(C)C)cc2)c1]

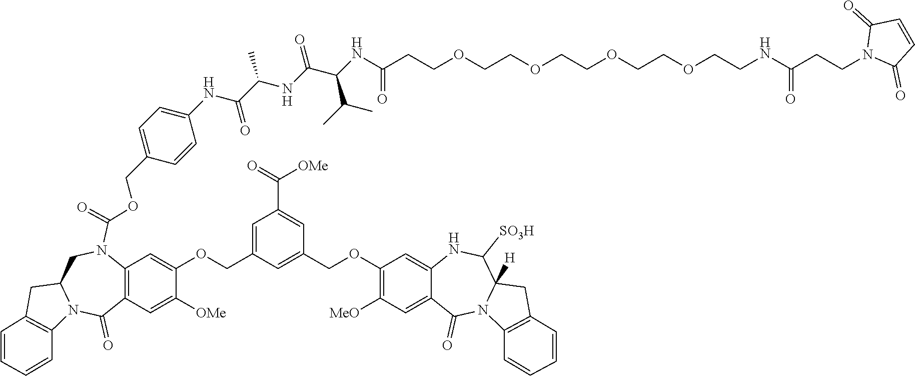 [SMILES: COC(=O)c1cc(COc2cc3c(cc2OC)C(=O)N2c4ccccc4C[C@H]2C(S(=O)(=O)O)N3)cc(COc2cc3c(cc2OC)C(=O)N2c4ccccc4C[C@H]2CN3C(=O)OCc2ccc(NC(=O)[C@H](C)NC(=O)[C@@H](NC(=O)CCOCCOCCOCCOCCNC(=O)CCN3C(=O)C=CC3=O)C(C)C)cc2)c1]

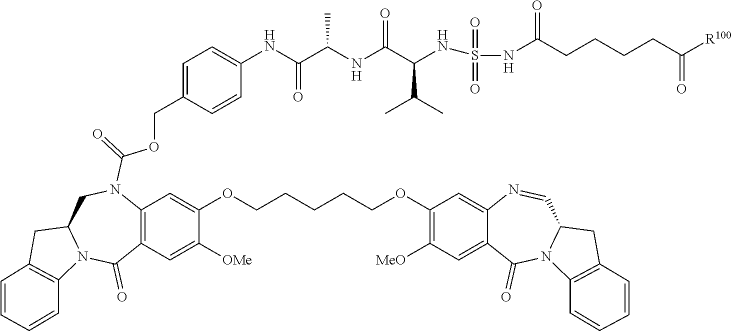

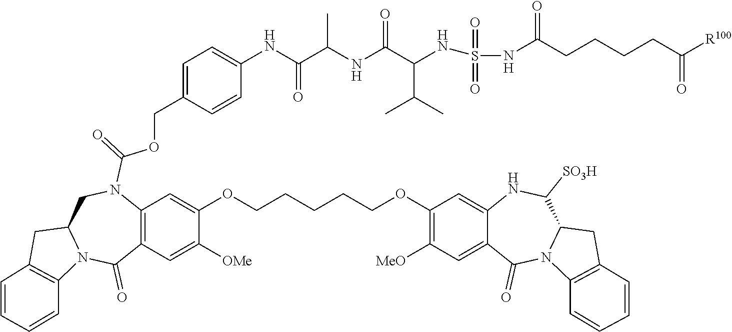

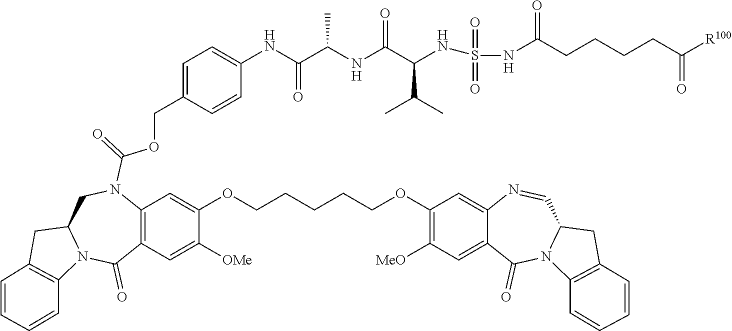

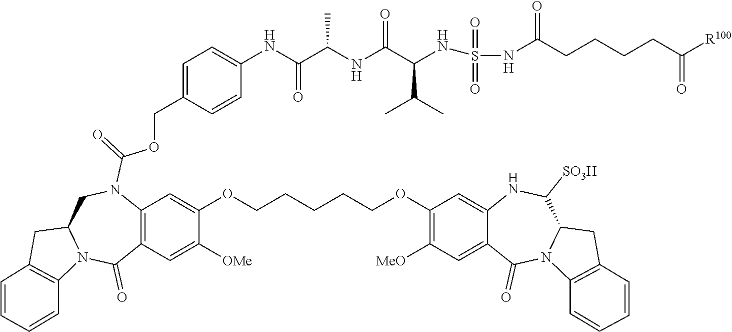

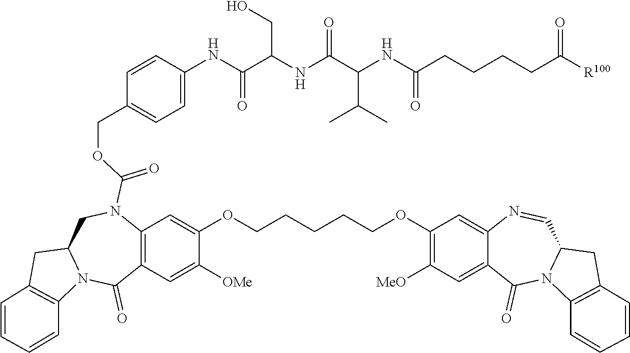

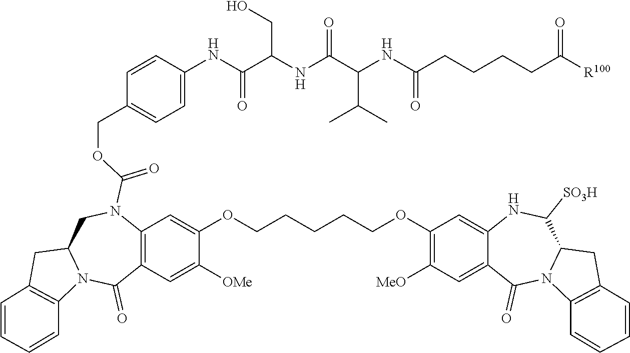

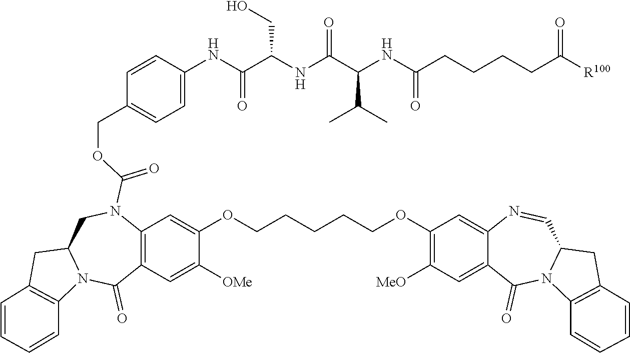

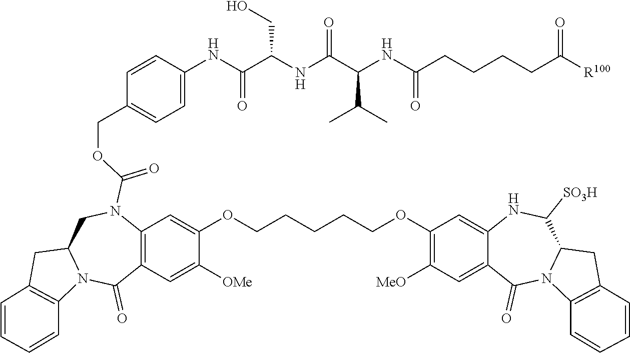

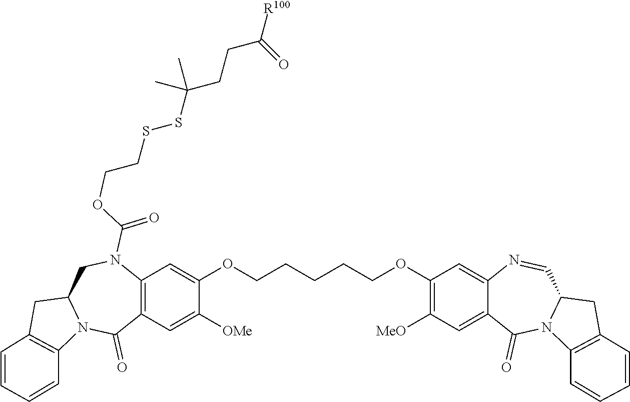

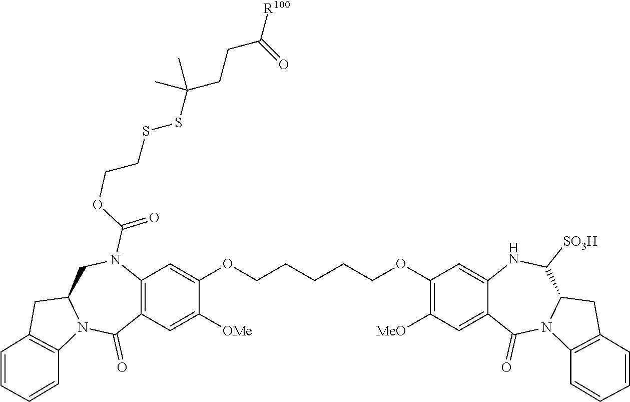

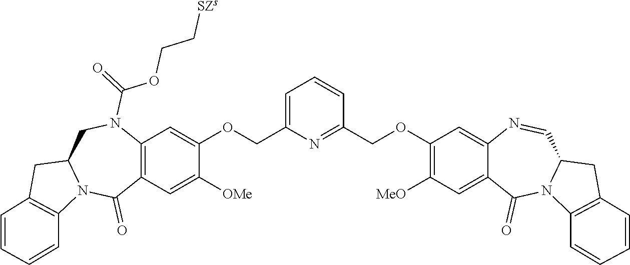

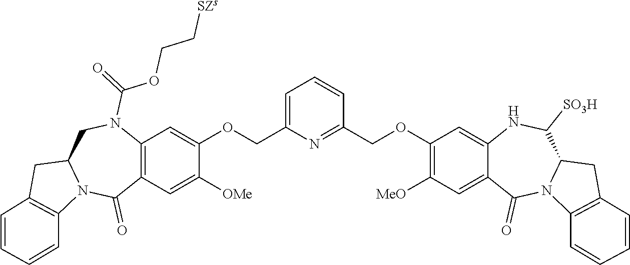

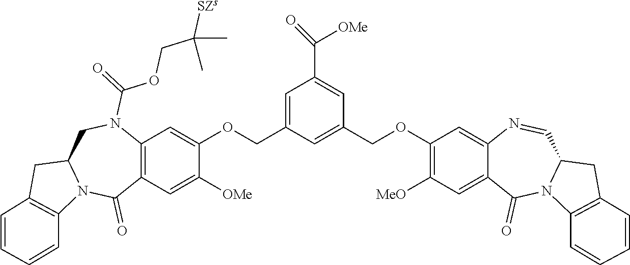

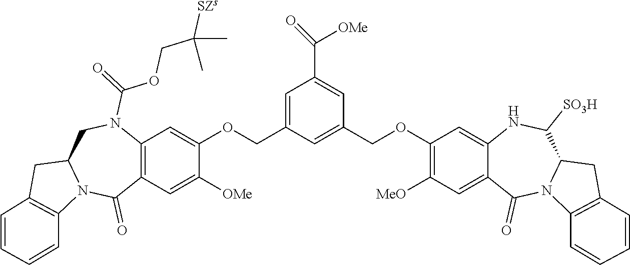

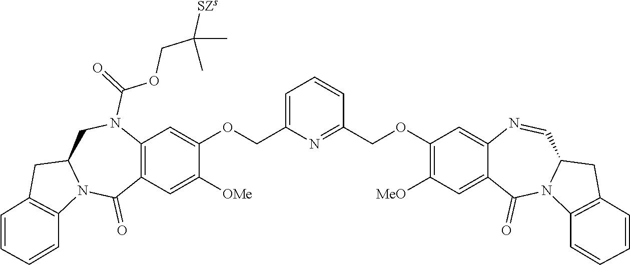

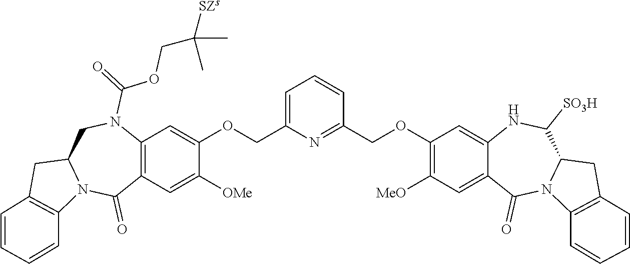

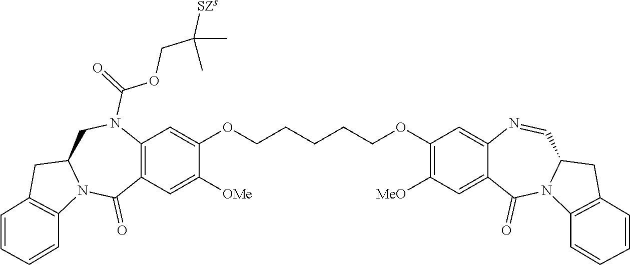

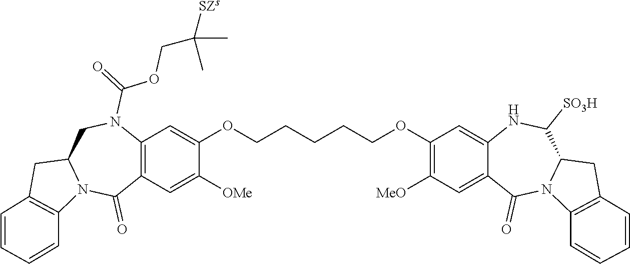

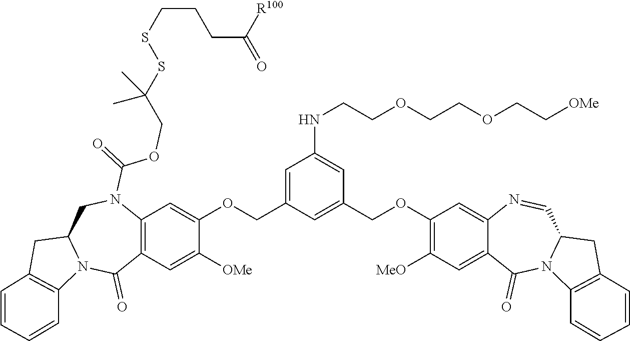

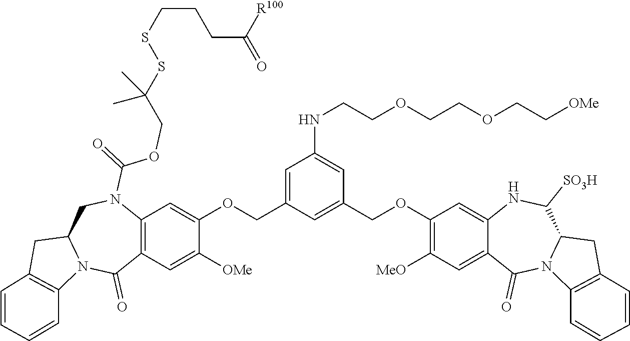

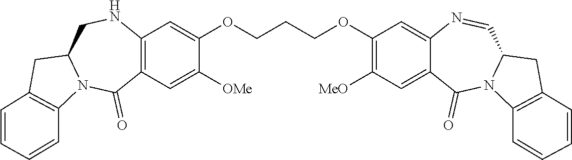 [SMILES: COc1cc2c(cc1OCCCOc1cc3c(cc1OC)C(=O)N1c4ccccc4C[C@H]1CN3)N=C[C@@H]1Cc3ccccc3N1C2=O]

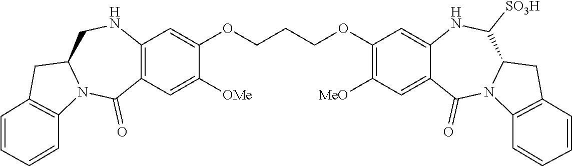

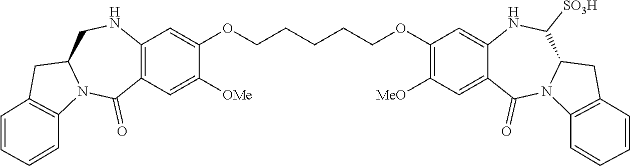

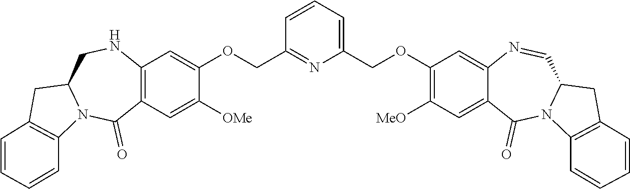 [SMILES: COc1cc2c(cc1OCc1cccc(COc3cc4c(cc3OC)C(=O)N3c5ccccc5C[C@H]3CN4)n1)N=C[C@@H]1Cc3ccccc3N1C2=O]

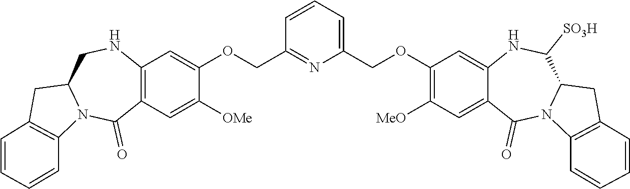

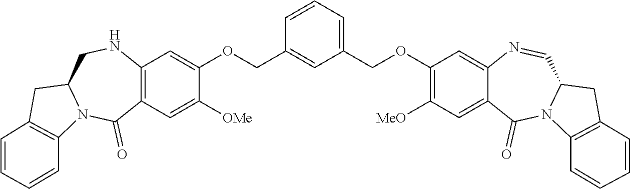 [SMILES: COc1cc2c(cc1OCc1cccc(COc3cc4c(cc3OC)C(=O)N3c5ccccc5C[C@H]3CN4)c1)N=C[C@@H]1Cc3ccccc3N1C2=O]

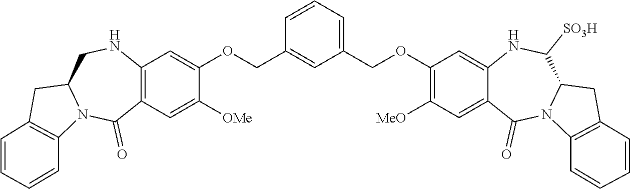

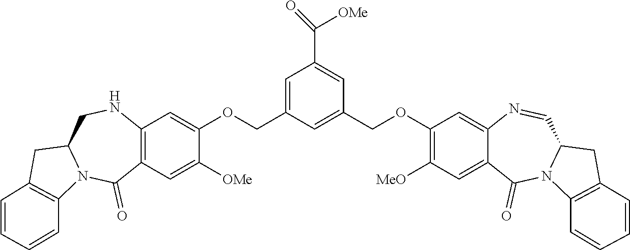 [SMILES: COC(=O)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

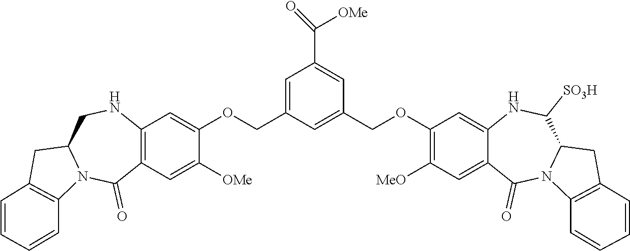

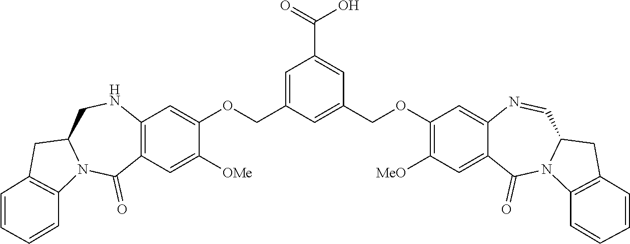 [SMILES: COc1cc2c(cc1OCc1cc(COc3cc4c(cc3OC)C(=O)N3c5ccccc5C[C@H]3CN4)cc(C(=O)O)c1)N=C[C@@H]1Cc3ccccc3N1C2=O]

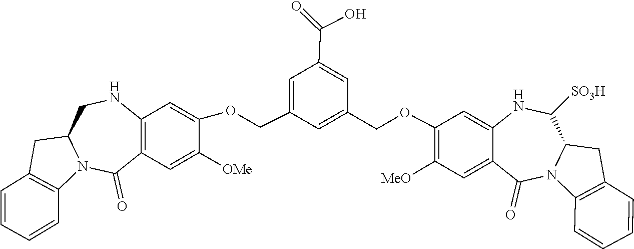

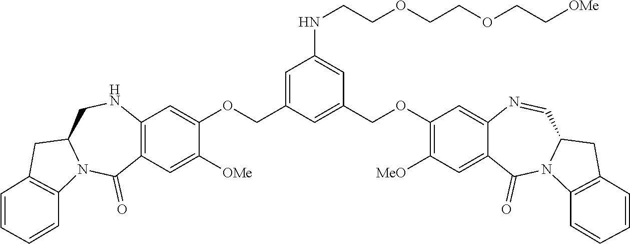 [SMILES: COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

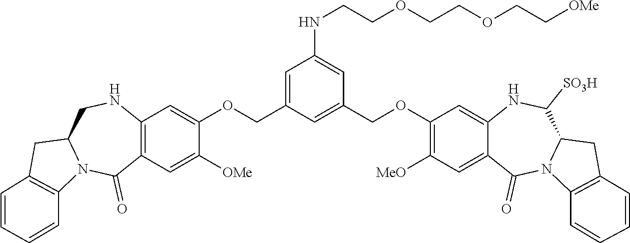

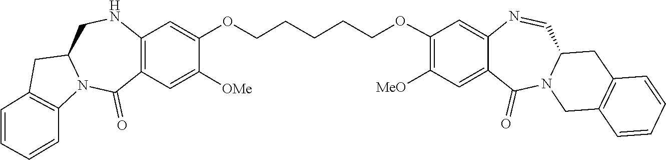 [SMILES: COc1cc2c(cc1OCCCCCOc1cc3c(cc1OC)C(=O)N1c4ccccc4C[C@H]1CN3)N=C[C@@H]1Cc3ccccc3CN1C2=O]

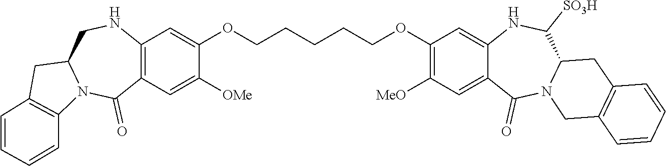

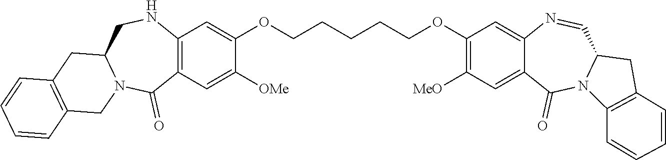 [SMILES: COc1cc2c(cc1OCCCCCOc1cc3c(cc1OC)C(=O)N1Cc4ccccc4C[C@H]1CN3)N=C[C@@H]1Cc3ccccc3N1C2=O]

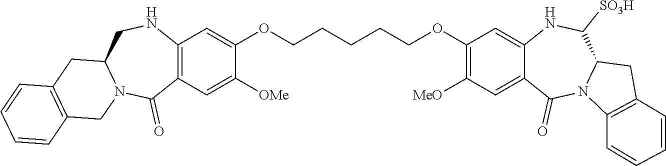

or a pharmaceutically acceptable salt thereof, wherein:

R¹⁰⁰ is —OH, —OMe or

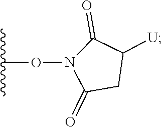

Z^{s} is H, SR^{e}, or is selected from the following formulae:

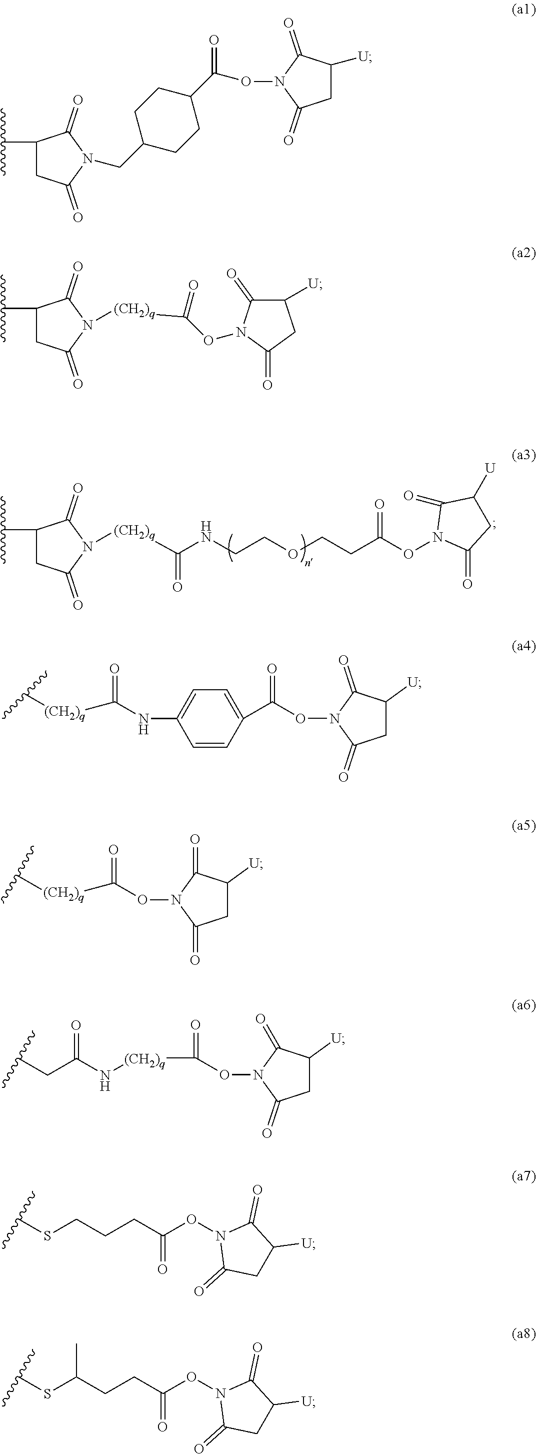

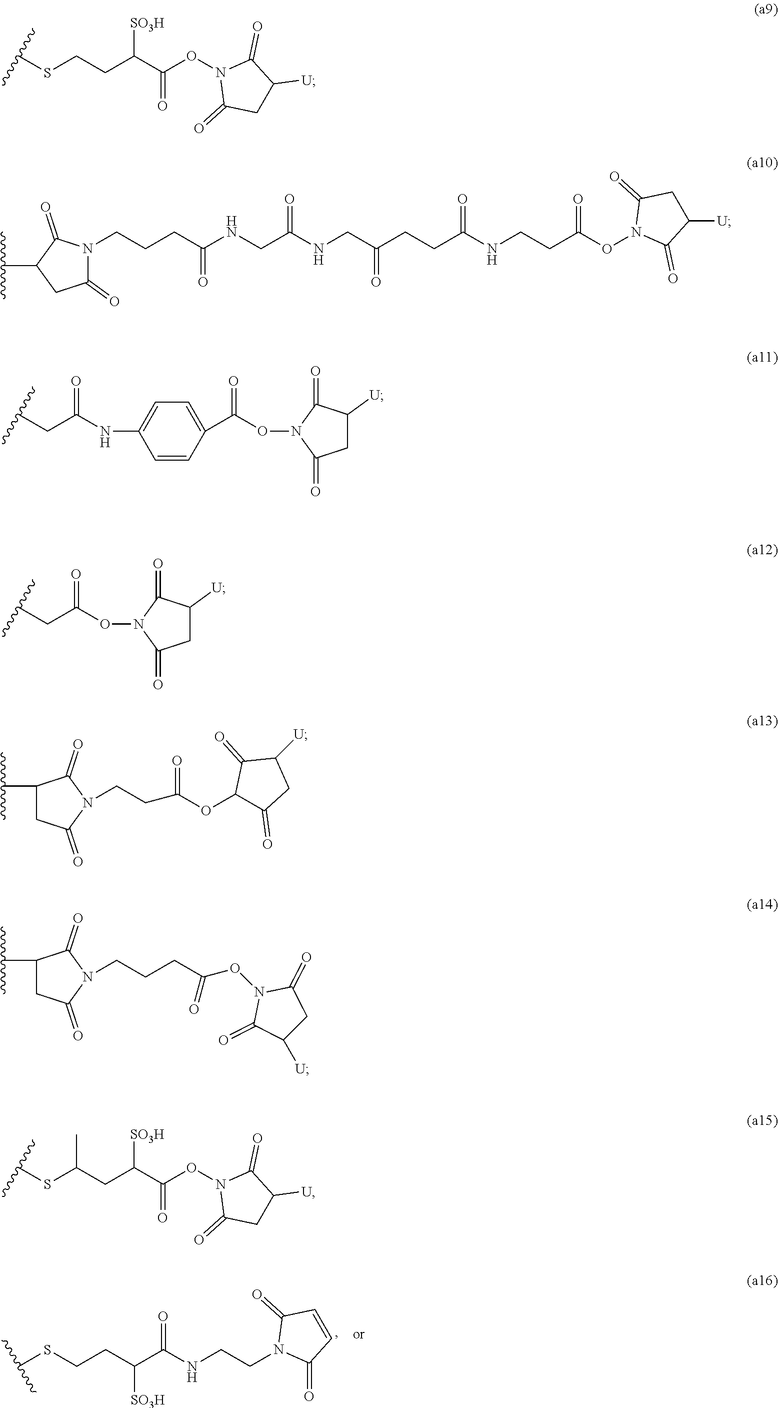

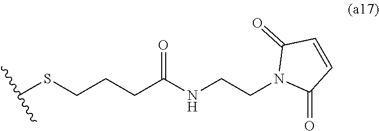

wherein:

q is an integer from 1 to 5;

n′ is an integer from 2 to 6;

U is —H or SO₃H;

R^{e} is a linear or branched alkyl having 1 to 6 carbon atoms or is selected from phenyl, nitrophenyl (e.g., 2 or 4-nitrophenyl), dinitrophenyl (e.g., 2,4-dinitrophenyl), carboxynitrophenyl (e.g., 3-carboxy-4-nitrophenyl), pyridyl or nitropyridyl (e.g., 4-nitropyridyl); and the remaining variables are as defined in the 1^{St} embodiment.

In a specific embodiment, R¹⁰⁰ is

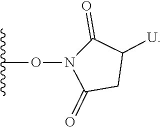

In a more specific embodiment, U is H.

In a 21^{st} embodiment, for compounds described above (e.g., compounds described in the first aspect or any embodiments described therein or in the 1^{St} to 20^{th} embodiments or any embodiments or specific embodiments described therein), the pharmaceutically acceptable salt thereof is a sodium or potassium salt. In one embodiment, the pharmaceutically acceptable salt is a sodium salt. In one embodiment, the pharmaceutically acceptable salt is a potassium salt.

### Conjugates of the Present Invention

In a second aspect, the present invention provides a cell-binding agent-cytotoxic agent conjugate comprising a cell-binding agent described herein covalently linked to a cytotoxic compound described herein.

In a 22^{nd} embodiment, the conjugate of the present invention is represented by the following formula:

CBA-(-Cy)_{w},

or a pharmaceutically acceptable salt thereof, wherein:

CBA is a cell-binding agent;

Cy is a cytotoxic agent represented by the following formula:

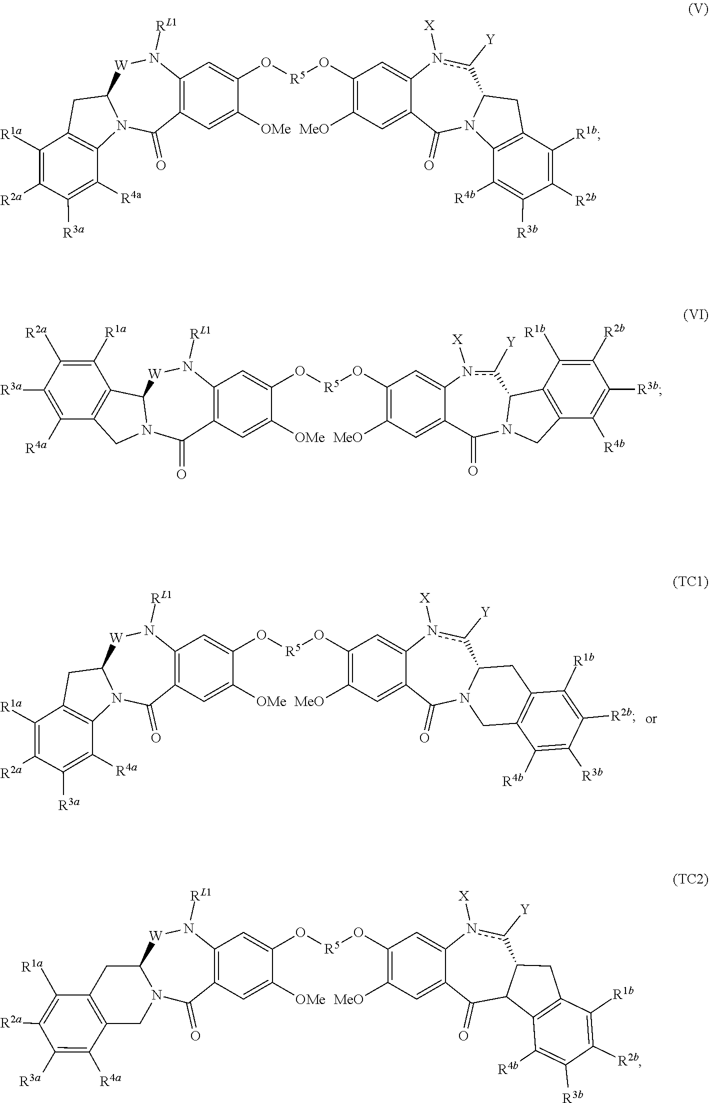

or a pharmaceutically acceptable salt thereof, wherein:

the double line [symbol] between N and C represents a single bond or a double bond, provided that when it is a double bond X is absent and Y is H, or a C₁₋₄alkyl, and when it is a single bond, X is H and Y is —OH or —SO₃H;

W is —C(═O)— or —C(Y′)—;

Y′ is H or C₁₋₄alkyl;

R^{1a}, R^{2a}, R^{3a}, R^{4a}, R^{1b}, R^{2b}, R^{3b} and R^{4b} are each independently selected from the group consisting of H, a C₁₋₁₀alkyl, —(OCH₂CH₂)ₙ—OR^{c}, halogen, —NH(C═NH)NH₂, —OR, —NR′R″, —NO₂, —NR′COR″, —SR, —SOR′, —SO₂R′, —SO₃H, —OSO₃H, —SO₂NR′R″, —CN, —N₃, —COR′, —OCOR′, and —OCONR′R″;

R^{c} is H or a C₁₋₄alkyl;

n is an integer from 1 to 24;

R, for each occurrence, is independently selected from the group consisting of H, —(CH₂CH₂O)ₙ—R^{c}, C₁₋₁₀alkyl, a C₃₋₈cycloalkyl, a 6- to 18-membered aryl, a 5- to 18-membered heteroaryl ring containing one or more heteroatoms independently selected from N, O and S, or a 3- to 18-membered heterocyclic ring containing 1 to 6 heteroatoms independently selected from O, S, N and P;

R′ and R″ are each independently selected from —H, —OH, —OR, —NHR, —NR₂, —COR, a C₁₋₁₀alkyl, a-(CH₂CH₂O)ₙ—R^{c}, and a 3- to 18-membered heterocyclic ring having 1 to 6 heteroatoms independently selected from O, S, N and P;

R⁵ is a C₃₋₁₂alkylene, which chain can be interrupted by one or more groups selected from —O—, —S—, —NH—, —NMe-, benzene ring, a 4 to 7-membered heteroaryl ring and a 4 to 7-membered heterocyclic ring, wherein the benzene, the 4 to 7-membered heteroaryl ring and the 4 to 7-membered heterocyclic ring are substituted with 1 to 4 R⁶;

R⁶ for each occurrence is independently selected from H, C₁₋₁₀alkyl, —(CH₂CH₂O)ₙ—R^{c}, halogen, —NH(C═NH)NH₂, —OR, —NR′R″, —NO₂, —NCO, —NR′COR″, —SR, —SOR′, —SO₂R′, —SO₃H, —OSO₃H, —SO₂NR′R″, —CN, —N₃, —COR′, —OCOR′, and —OCONR′R″; and

R^{L1} is a self-immolative linker covalently linked to the CBA, provided the conjugate of formula (V) is not:

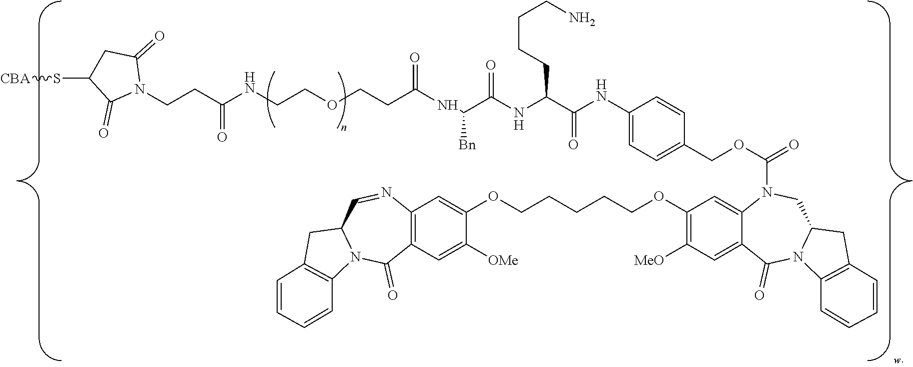

In a 23^{rd} embodiment, for conjugates of formula (V), (VI), (TC1) or (TC2) described in the 22^{nd} embodiment, or a pharmaceutically acceptable salt thereof, wherein Cy is represented by one of the following formulae in Table E:

| | |
|---|---|
| 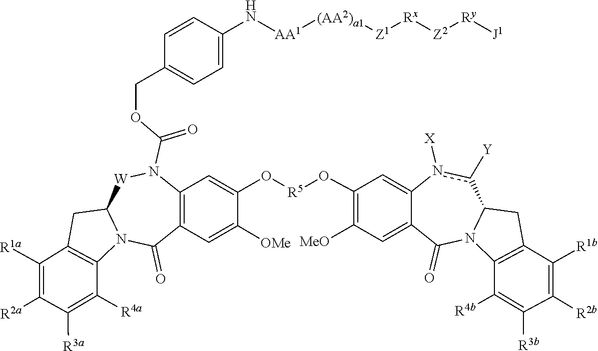 | (VIIa) |
| 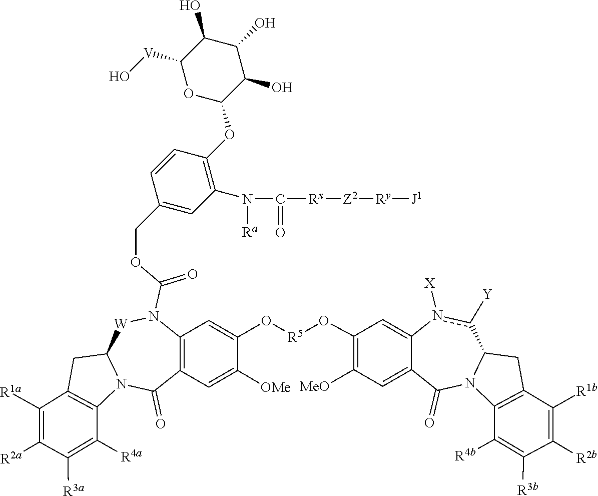 | (VIIb) |
| 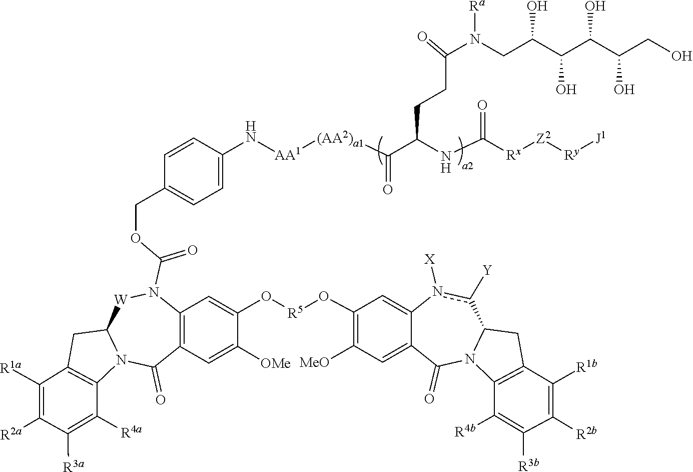 | (VIIc) |
| 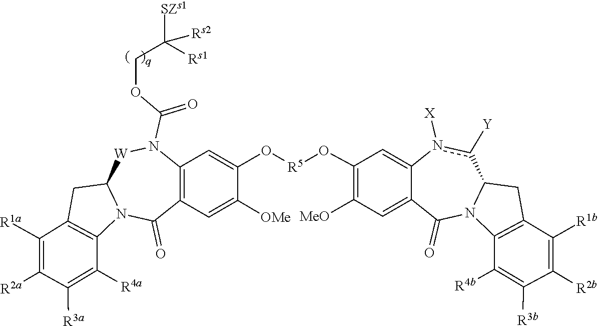 | (VIId) |
| 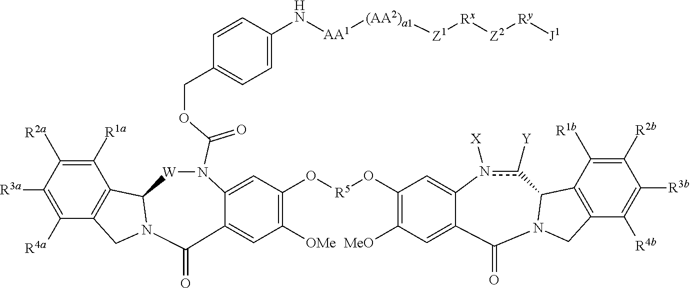 | (VIIIa) |
| 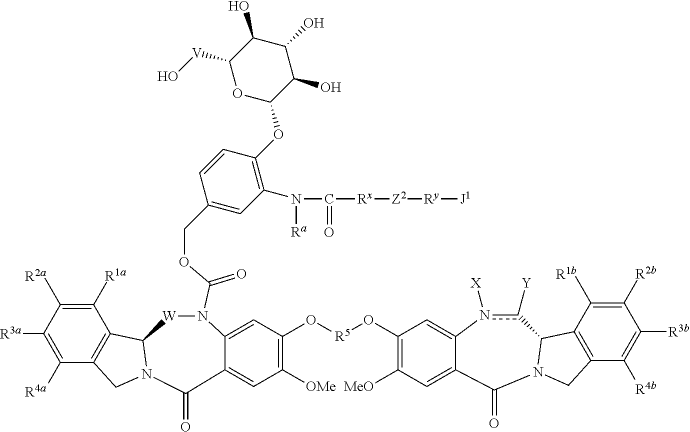 | (VIIIb) |
| 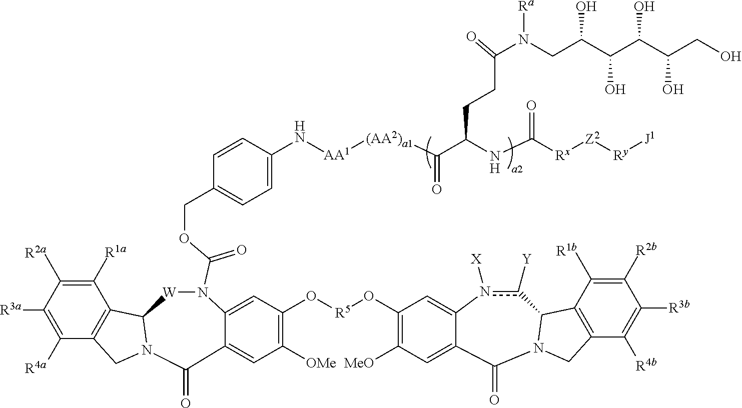 | (VIIIc) |
| 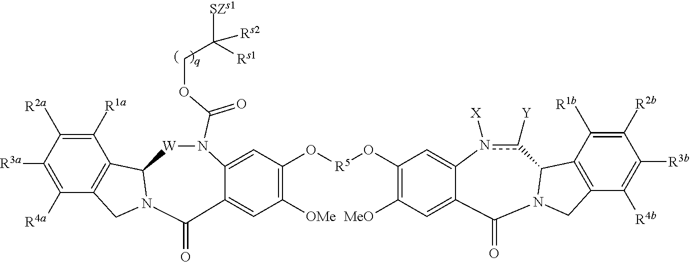 | (VIIId) |
| 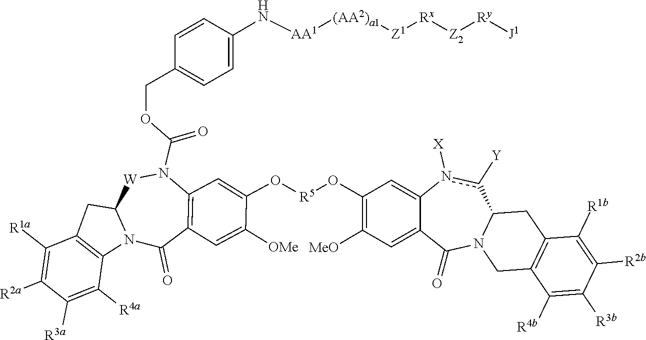 | (TC1a) |
| 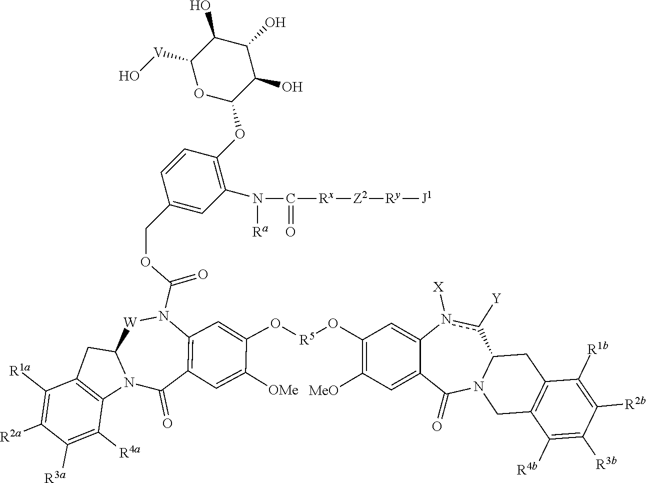 | (TC1b) |
| 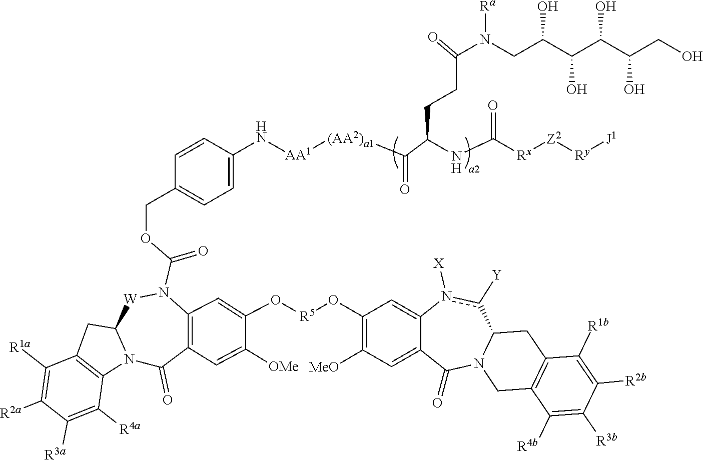 | (TC1c) |
| 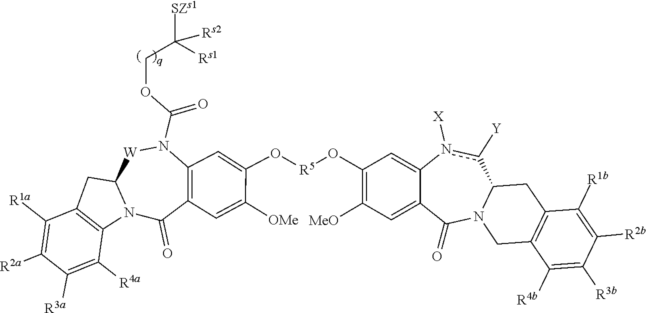 | (TC1d) |
| 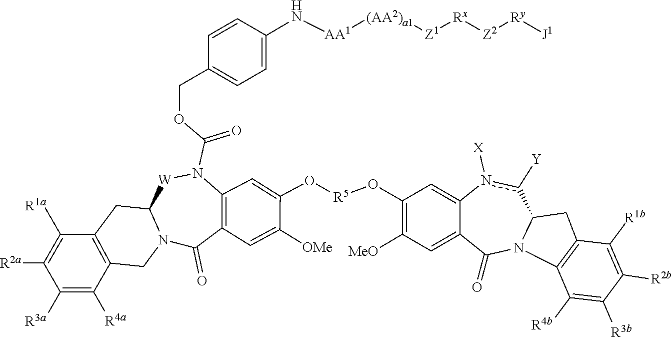 | (TC2a) |
| 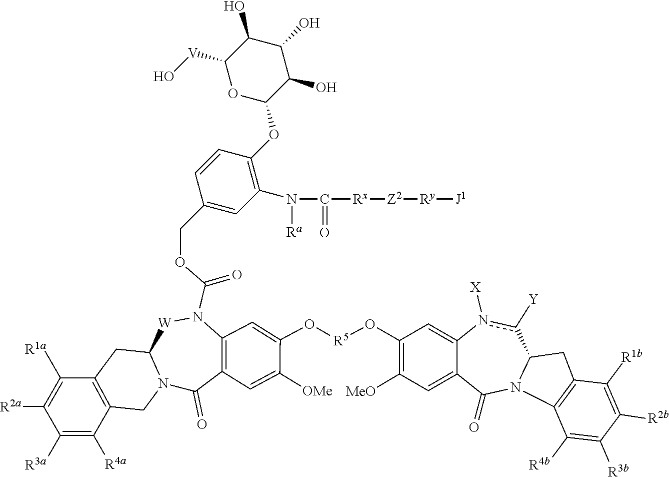 | (TC2b) |
| 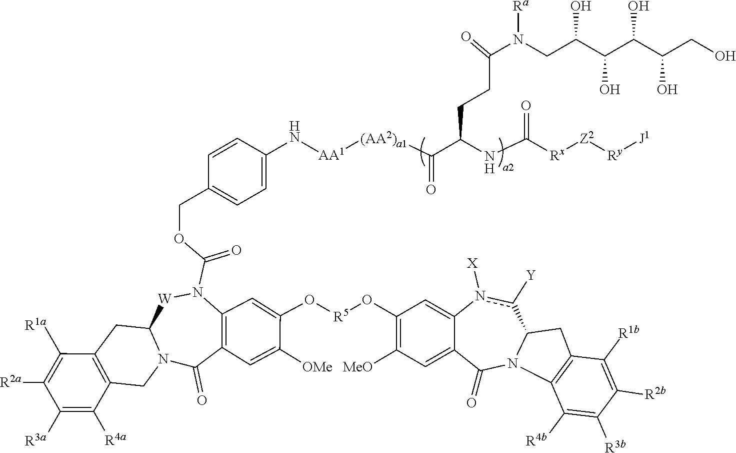 | (TC2c) |
| 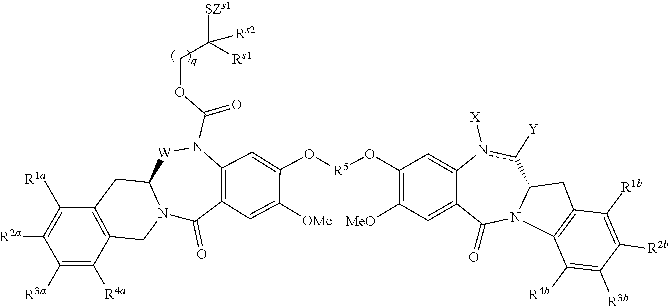 | (TC2d) |

or a pharmaceutically acceptable salt thereof, wherein:

AA¹ and AA² are each independently an amino acid residues;

a1 is an integer from 1 to 19;

a2 is an integer from 1 to 5;

R^{a} is H or C₁₋₄alkyl;

q is 1, 2, 3 or 4;

R^{s1} and R^{s2} are each independently H or C₁₋₄alkyl, or R^{s1} and R^{s2} taken together with the carbon atom to which they are attached form a 3 to 5-membered cycloalkyl ring, provided when q is 1, R^{s1} and R^{s2} taken together with the carbon atom to which they are attached form a 4 or 5-membered cycloalkyl ring;

V is C(═O) or CH₂;

Z¹ is —C(═O)— or —SO₂—NH—C(═O)—, wherein the —SO₂— group in —SO₂—NH—C(═O)— is connected to P¹;

R^{x} is absent, C₁₋₁₀alkylene, C₃₋₈cycloalkyl, —(CH₂CH₂O)ₘ₁—C₁₋₁₀alkylene- or C₁₋₁₀-alkylene-(OCH₂CH₂)ₘ₂—;

m1 and m2 are each independently an integer from 1 to 24;

Z² is absent, —C(═O)NH— or —NH—C(═O)—;

R^{y} is absent, C₁₋₁₀alkylene, —(CH₂CH₂O)ₘ₃—C₁₋₁₀alkylene- or C₁₋₁₀alkylene-(OCH₂CH₂)ₘ₄⁻;

m3 and m4 are each independently an integer from 1 to 24;

Z^{s1} is a bifunctional crosslinker that is covalently linked to the CBA and the cytotoxic compound, wherein the crosslinker is covalently linked to the cytotoxic compound via a disulfide bond or a thioether bond;

J¹ is a moiety formed by reacting an amine reactive group or a thiol reactive group of the cytotoxic agent with an amine group or a thiol group located on CBA; and the remaining variables are as defined in the second aspect or the 22^{nd} embodiment.

In a specific embodiment, for formula (VIId), (VIIId), (TC1d) or (TC2d), q is 1. In a more specific embodiment, for formula (VIId), (VIIId), (TC1d) or (TC2d), q is 1; and R^{s1} and R^{s2} are both methyl.

In another specific embodiment, for formula (VIIb), (VIIc), (VIIIb), (VIIIc), (TC1b), (TC1c), (TC2b) or (TC2c), R^{a} is H, methyl or ethyl. In a more specific embodiment, R^{a} is H. In a more specific embodiment, R^{a} is methyl.

In a 24^{th} embodiment, for conjugates described in the 22^{nd} embodiment, or a pharmaceutically acceptable salt thereof, wherein R^{1a}, R^{2a}, R^{3a}, R^{4a}, R^{1b}, R^{2b}, R^{3b} and R^{4b} are all H; and the remaining variables are as defined in the second aspect or the 22^{nd} or 23^{rd} embodiment or any specific embodiment described therein.

In a 25^{th} embodiment, for conjugates of the 22^{nd} or 23^{rd} embodiment, or a pharmaceutically acceptable salt thereof, wherein R⁵ is a C₃₋₇alkylene; and the remaining variables are as defined in the second aspect or the 22^{nd}, 23^{rd}, or 24^{th} embodiment or any specific embodiments described therein. In a specific embodiment, R⁵ is —(CH₂)₃—, —(CH₂)₅— or —(CH₂)₇—. In a more specific embodiment, R⁵ is —(CH₂)₇—. In a more specific embodiment, R⁵ is —(CH₂)₅—. In a more specific embodiment, R⁵ is —(CH₂)₃—.

In a 26^{th} embodiment, for conjugates of the 22^{nd} or 23^{rd} embodiment, or a pharmaceutically acceptable salt thereof, wherein R⁵ is represented by the following formula:

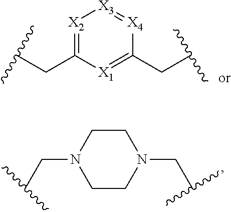

wherein X₁, X₂, X₃ and X₄ are each independently N or CR⁶, provided at least one of X₁, X₂, X₃ and X₄ is CR⁶; and the remaining variables are as defined in the second aspect or the 22^{nd}, 23^{rd}, or 24^{th} embodiment or any specific embodiment described therein.

In a specific embodiment, R⁵ is

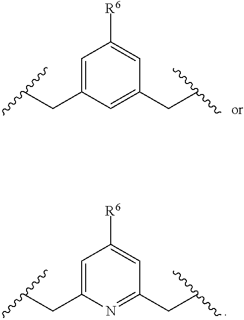

In a more specific embodiment, R⁵ is

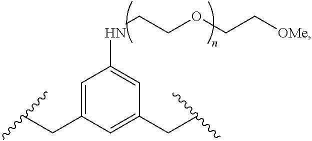

wherein n is an integer from 1 to 8. In a further specified embodiment, n is 1, 2, 3, or 4. In a more specific embodiment, n is 1. In a more specific embodiment, n is 2. In a more specific embodiment, n is 3. In a more specific embodiment, n is 4. In a more specific embodiment, R⁵ is

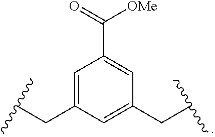 [SMILES: *Cc1cc(C*)cc(C(=O)OC)c1]

In a more specific embodiment, R⁵ is

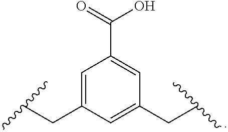 [SMILES: *Cc1cc(C*)cc(C(=O)O)c1]

In another more specific embodiment, R⁵ is

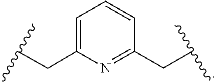 [SMILES: *Cc1cccc(C*)n1]

In a 27^{th} embodiment, for conjugates of the 23^{nd} embodiment, or a pharmaceutically acceptable salt thereof, Cy is represented by one of the following formulae in Table F:

| | |
|---|---|
| 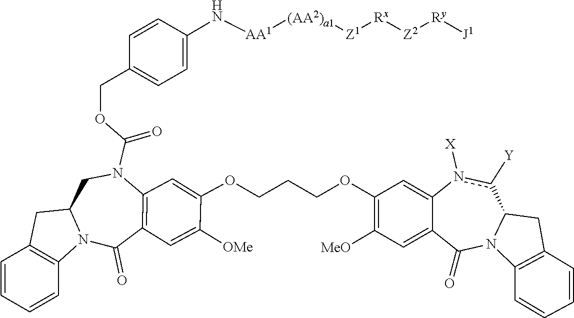 | (VIIaA) |
| 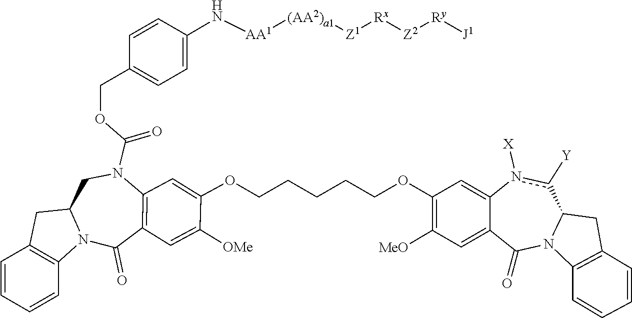 | (VIIaB) |
| 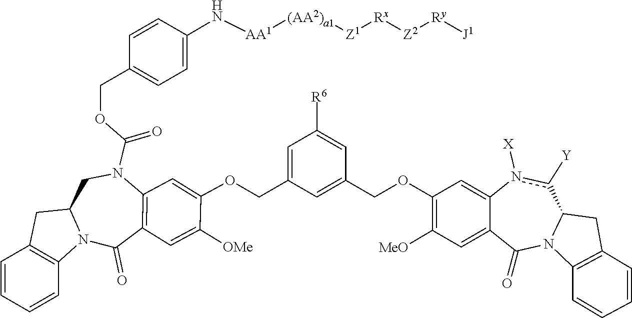 | (VIIaC) |
| 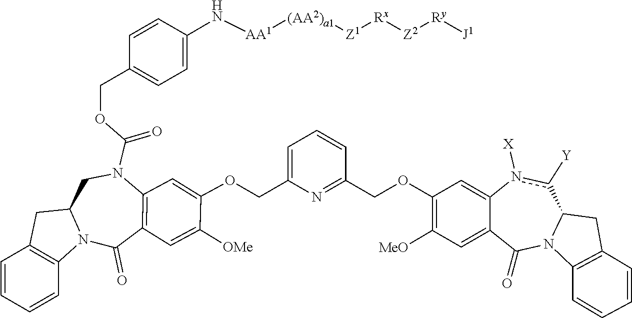 | (VIIaD) |
| 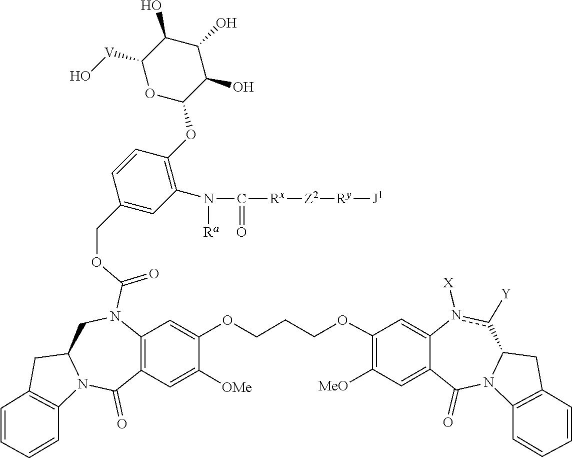 | (VIIbA) |
| 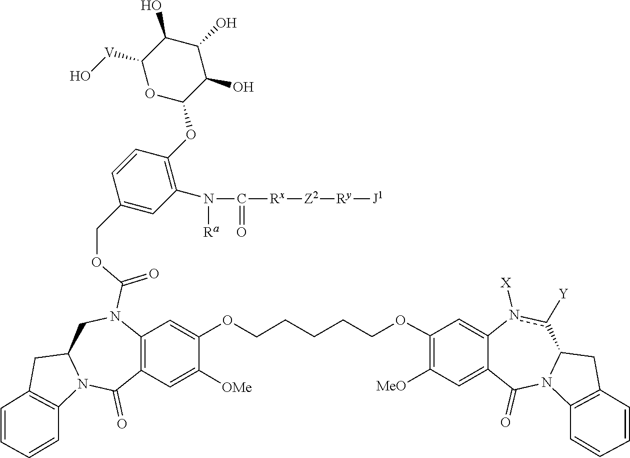 | (VIIbB) |
| 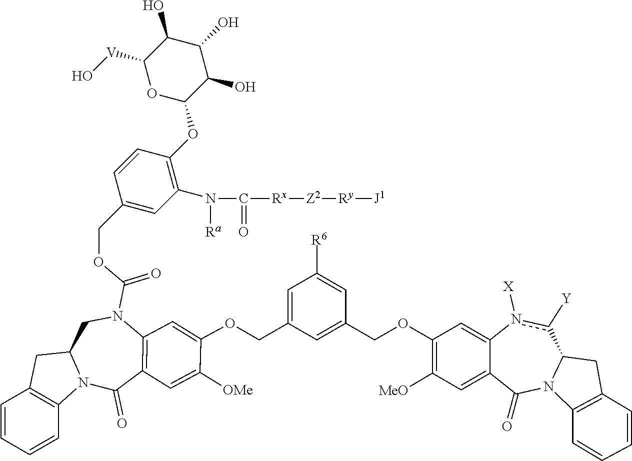 | (VIIbC) |
| 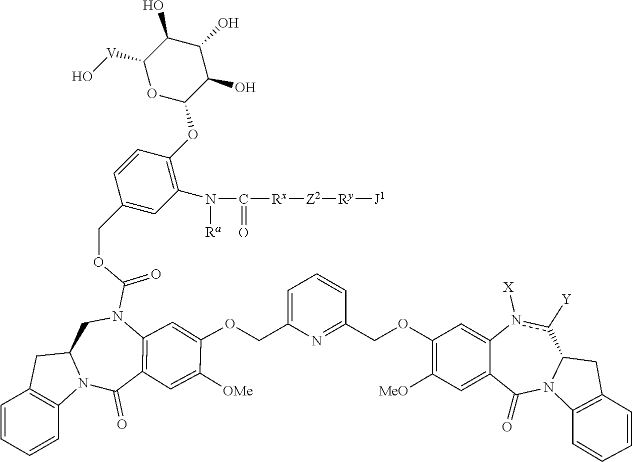 | (VIIbD) |
| 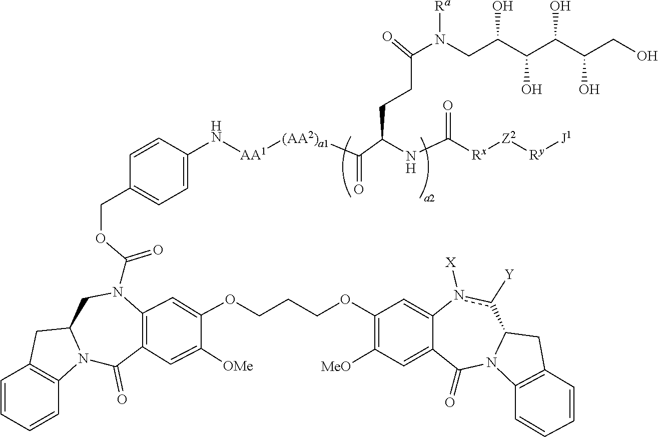 | (VIIcA) |
| 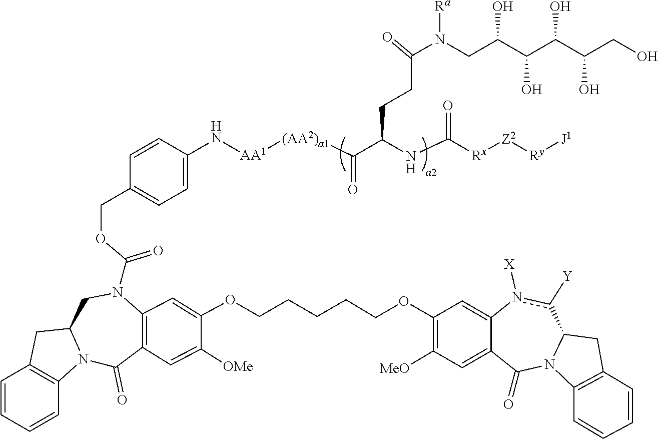 | (VIIcB) |
| 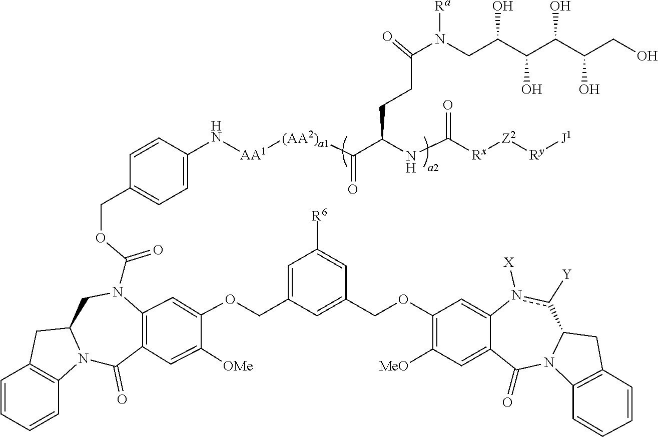 | (VIIcC) |
| 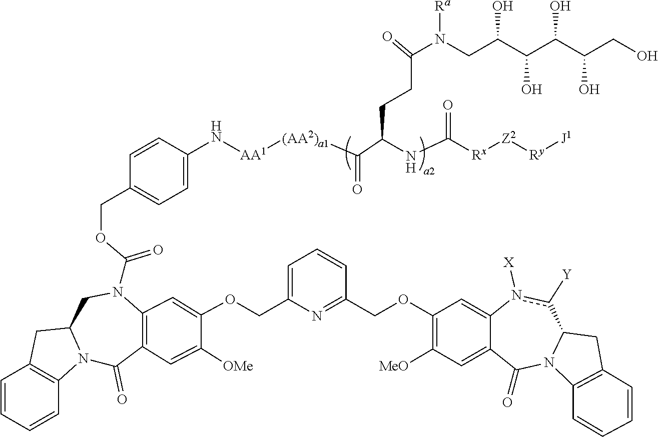 | (VIIcD) |
| 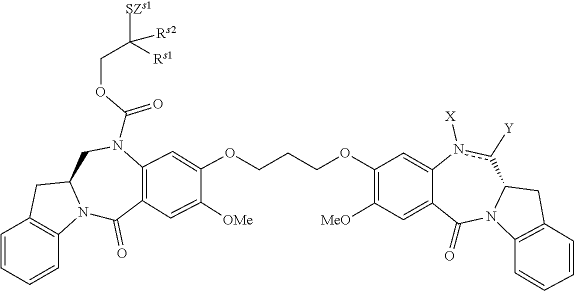 | (VIIdA) |
| 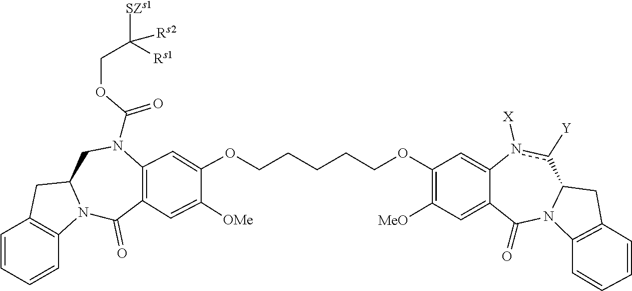 | (VIIdB) |
| 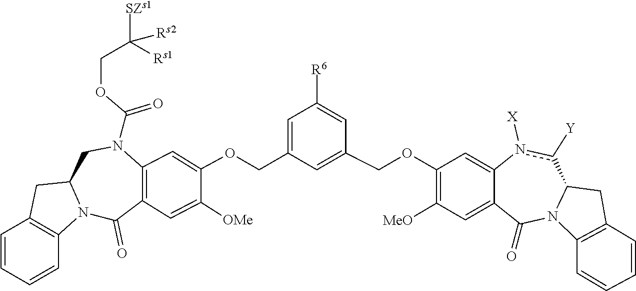 | (VIIdC) |
| 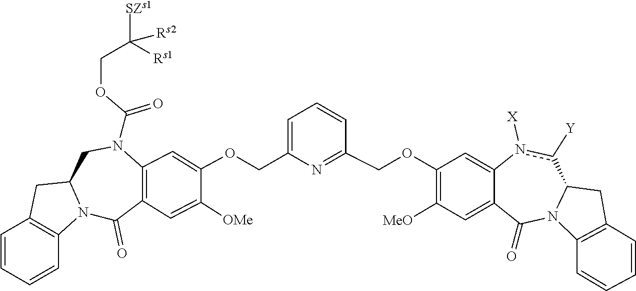 | (VIIdD) |
| 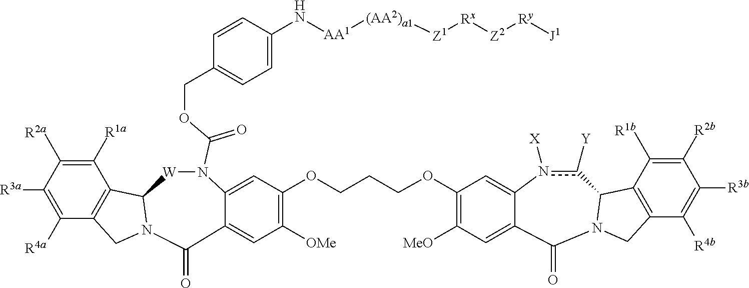 | (VIIIaA) |
| 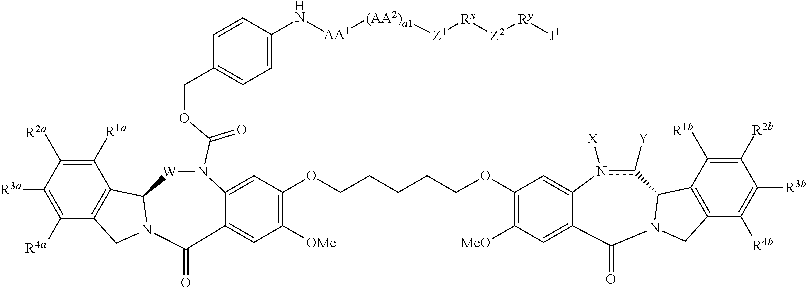 | (VIIIaB) |
| 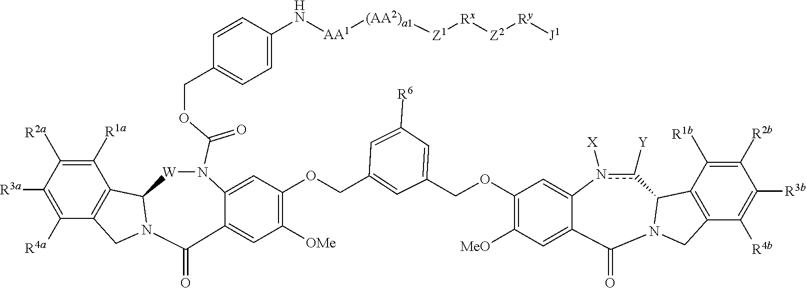 | (VIIIaC) |
| 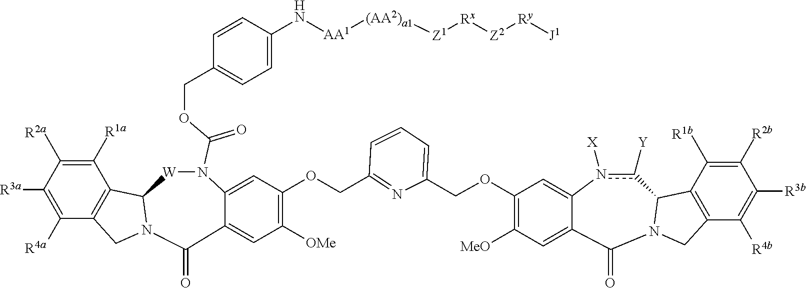 | (VIIIaD) |
| 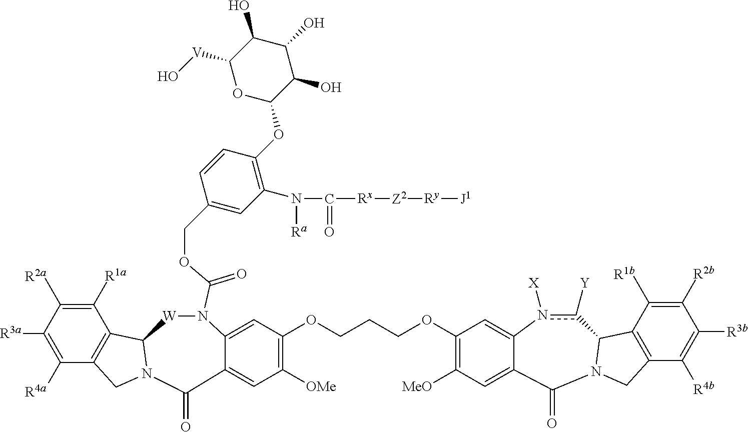 | (VIIIbA) |
| 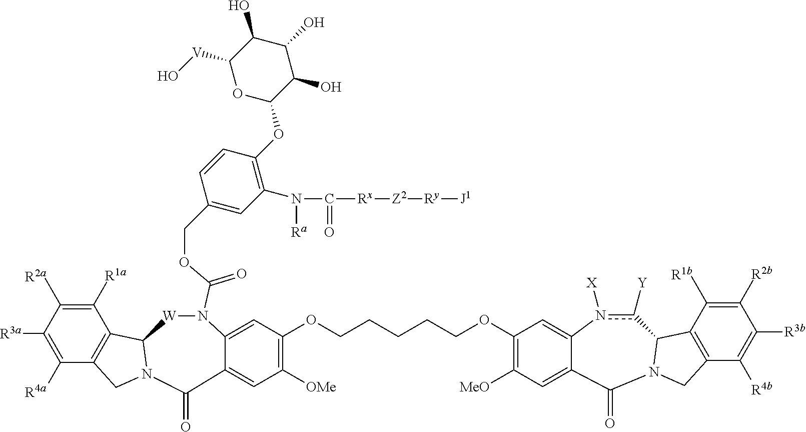 | (VIIIbB) |
| 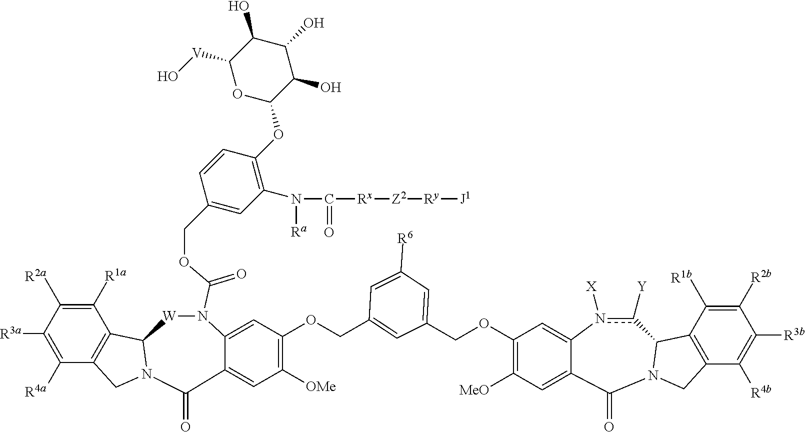 | (VIIIbC) |
| 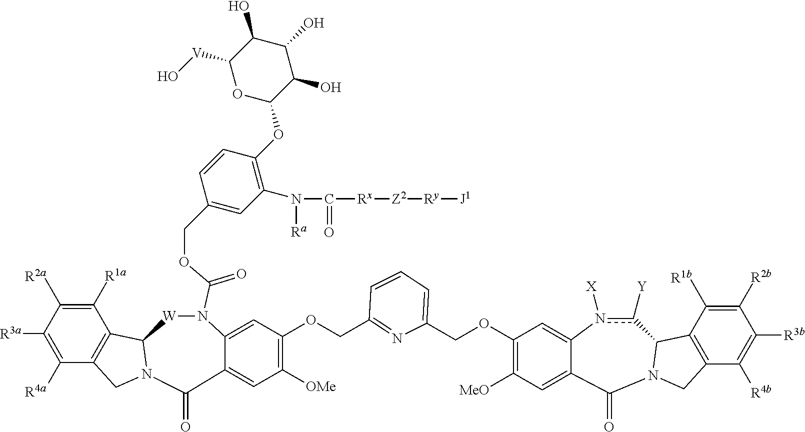 | (VIIIbD) |
| 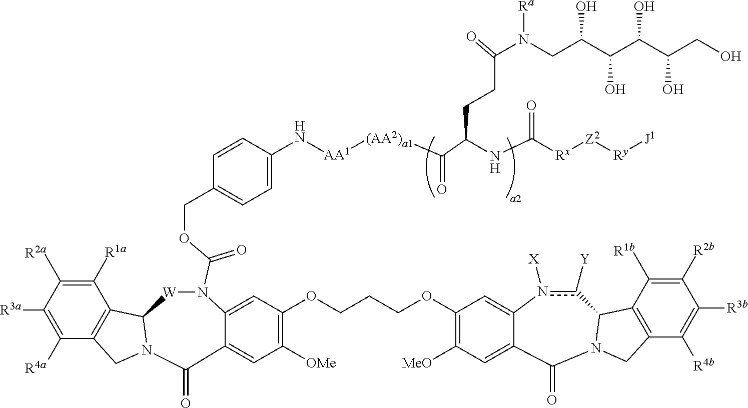 | (VIIIcA) |
| 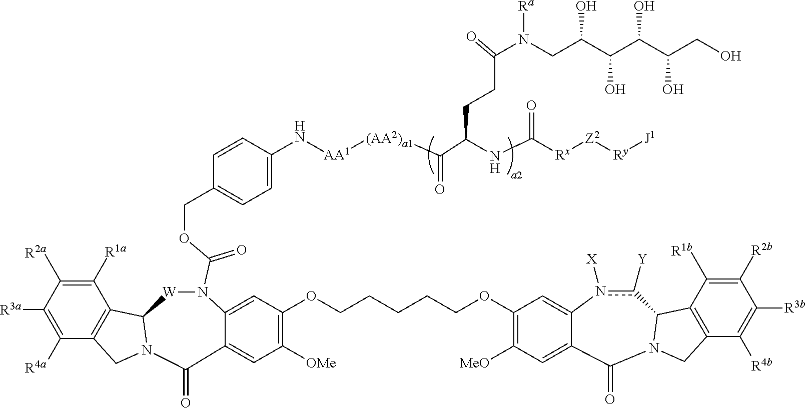 | (VIIIcB) |
| 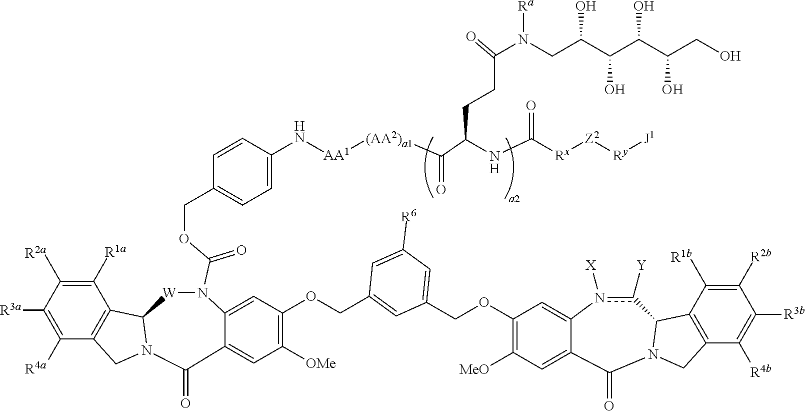 | (VIIIcC) |
| 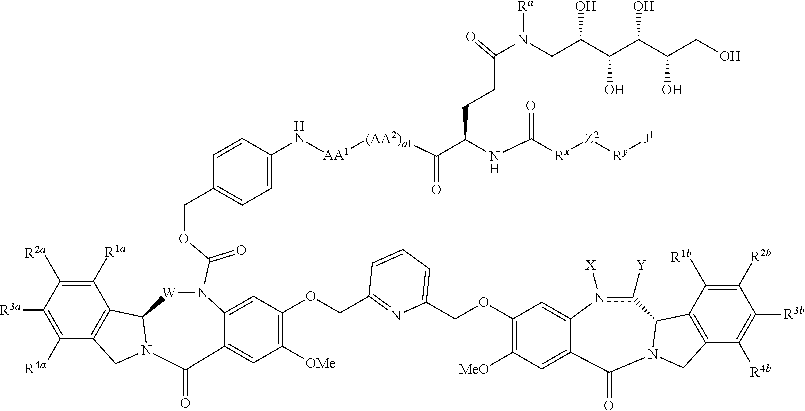 | (VIIIcD) |
| 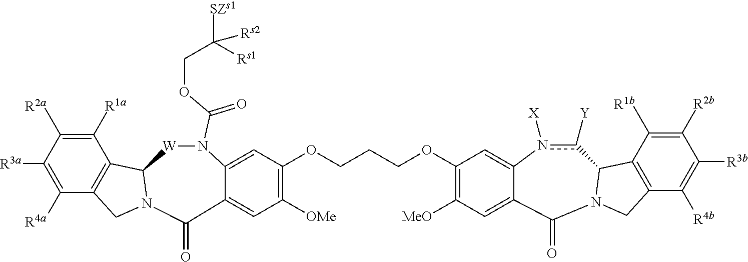 | (VIIIdA) |
| 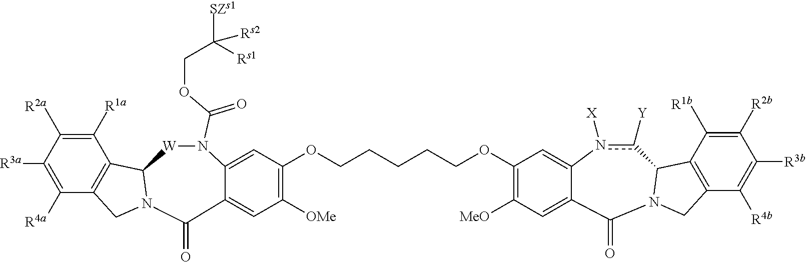 | (VIIIdB) |
| 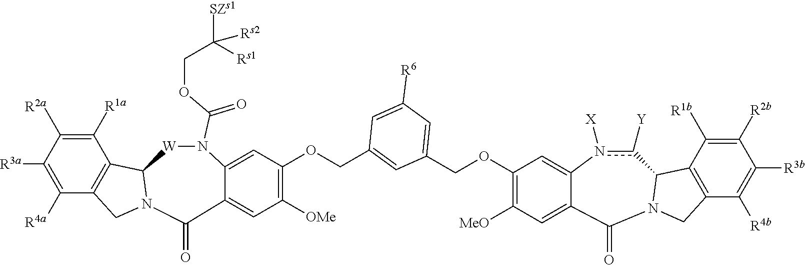 | (VIIIdC) |
| 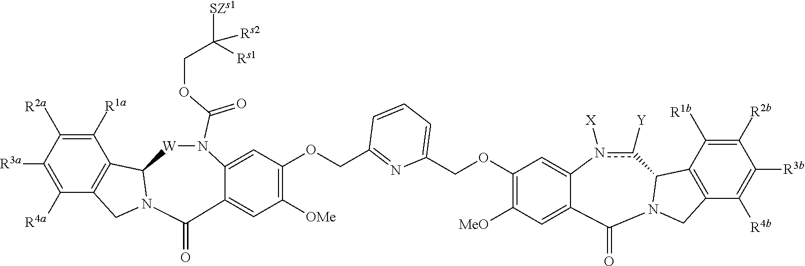 | (VIIIdD) |
| 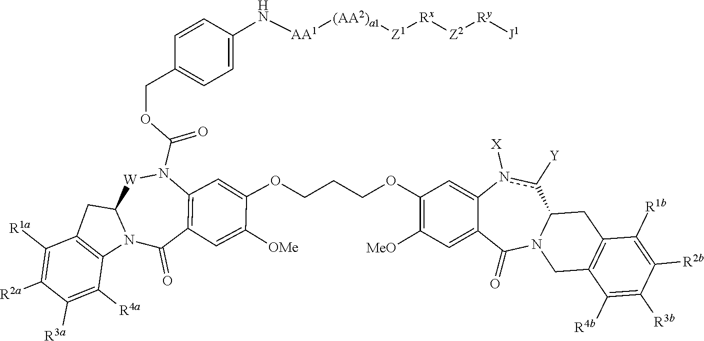 | (TC1aA) |
| 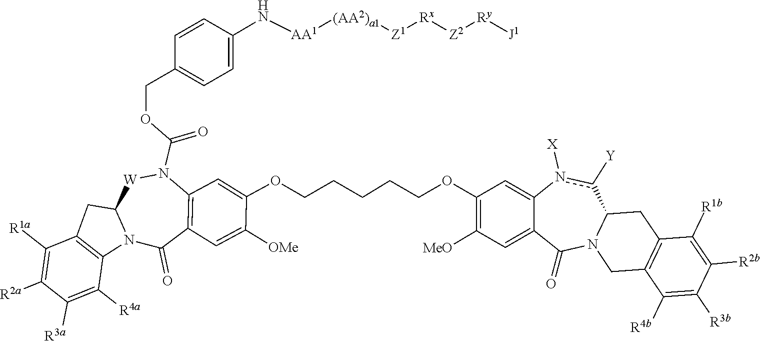 | (TC1aB) |
| 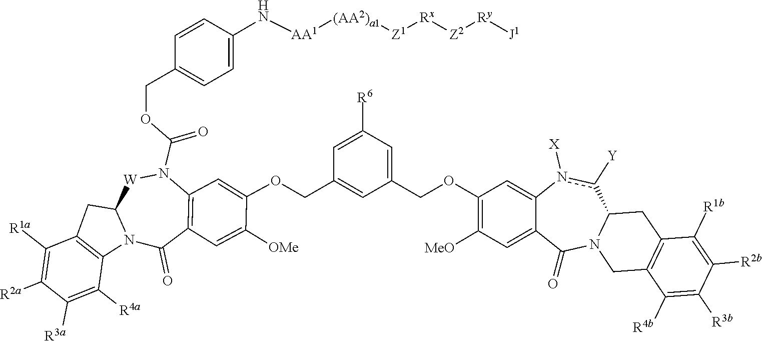 | (TC1aC) |
| 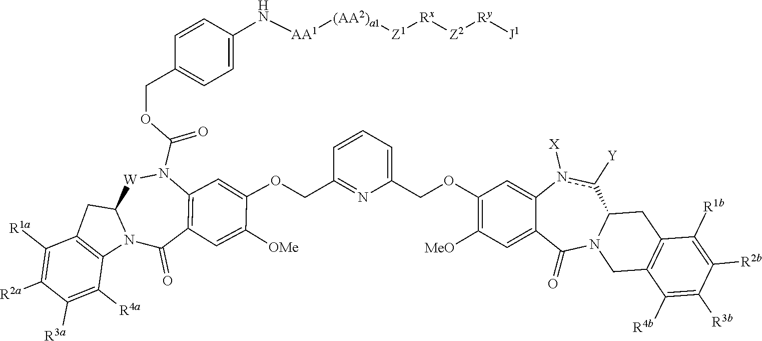 | (TC1aD) |
| 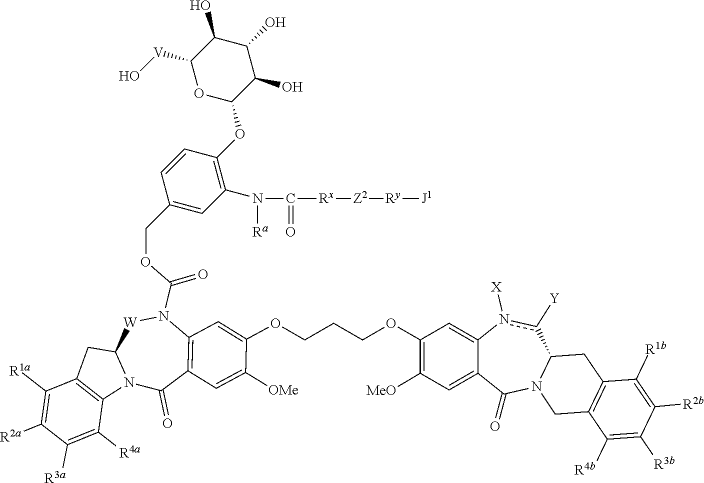 | (TC1bA) |
| 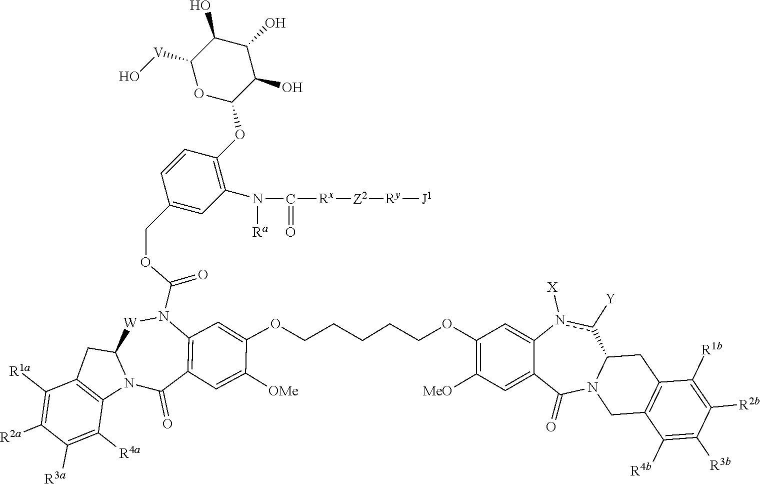 | (TC1bB) |
| 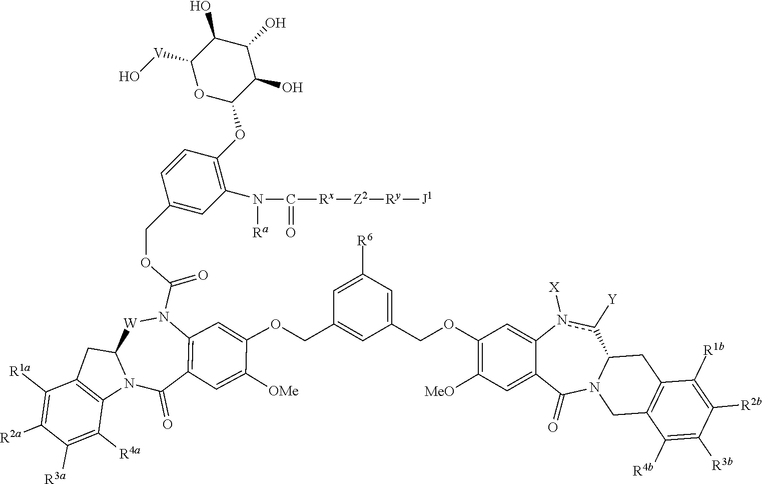 | (TC1bC) |
| 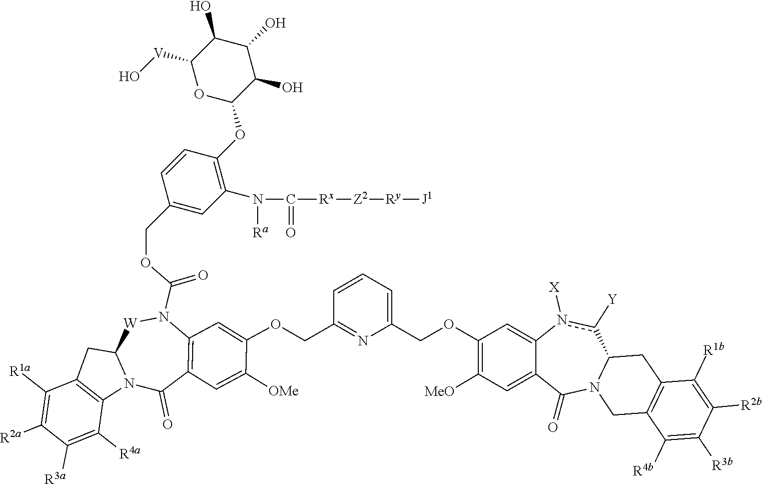 | (TC1bD) |
| 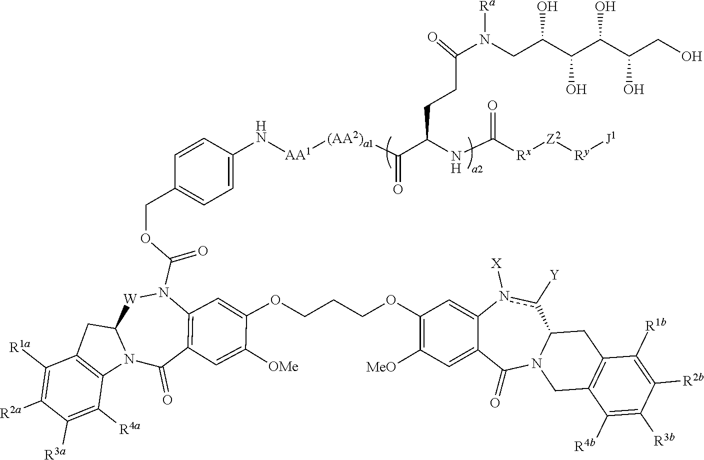 | (TC1cA) |
| 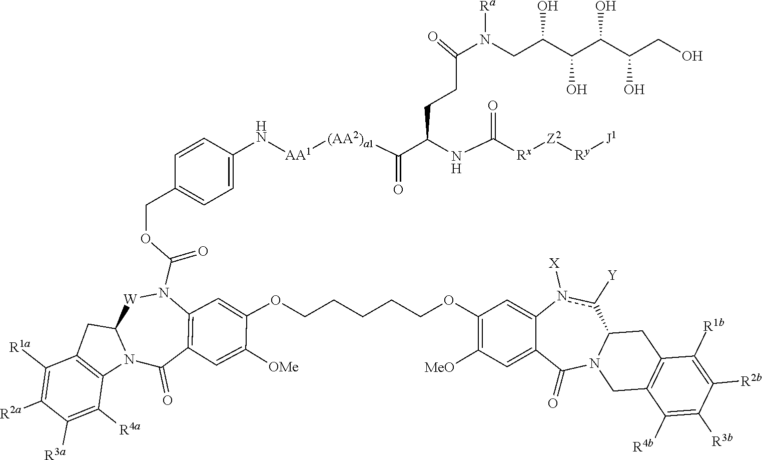 | (TC1cB) |
| 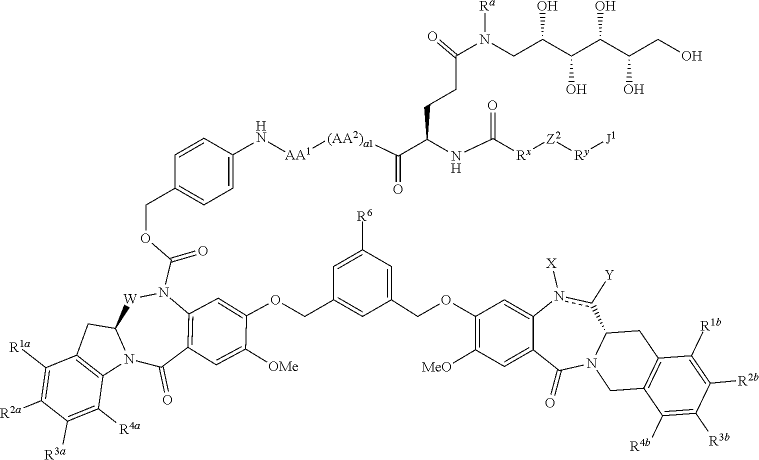 | (TC1cC) |
| 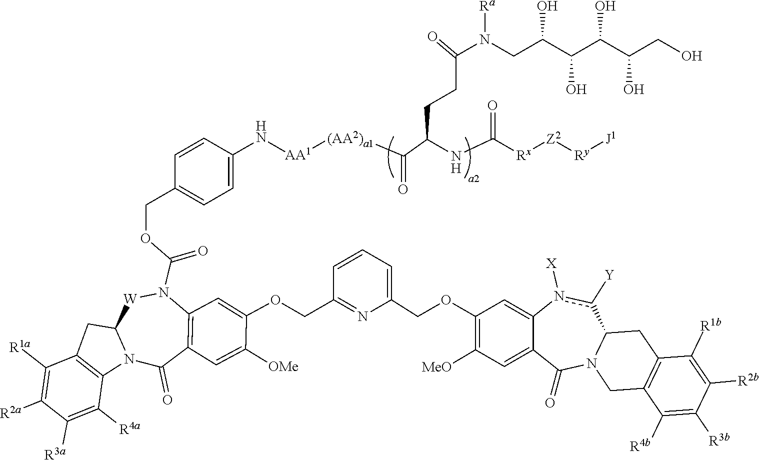 | (TC1cD) |
| 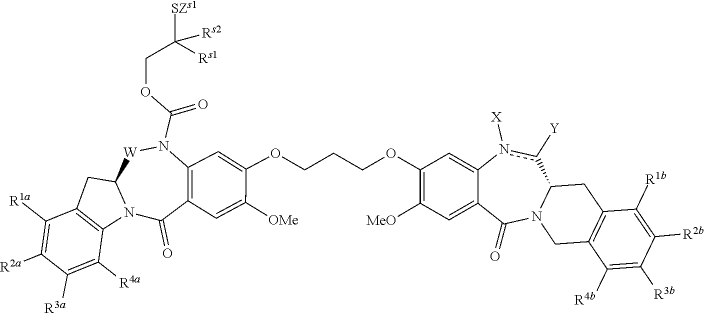 | (TC1dA) |
| 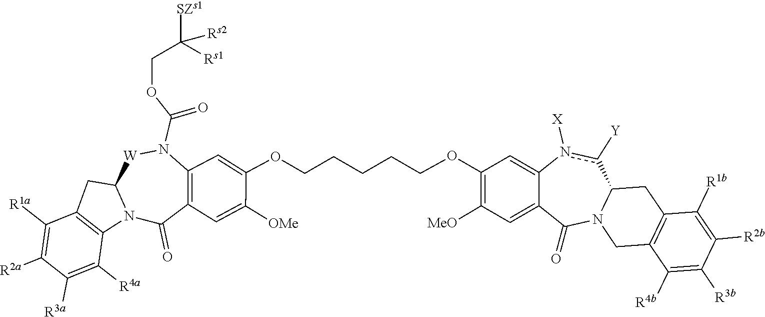 | (TC1dB) |
| 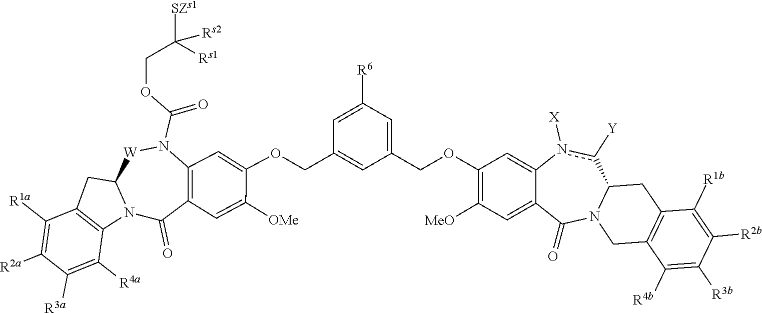 | (TC1dC) |
| 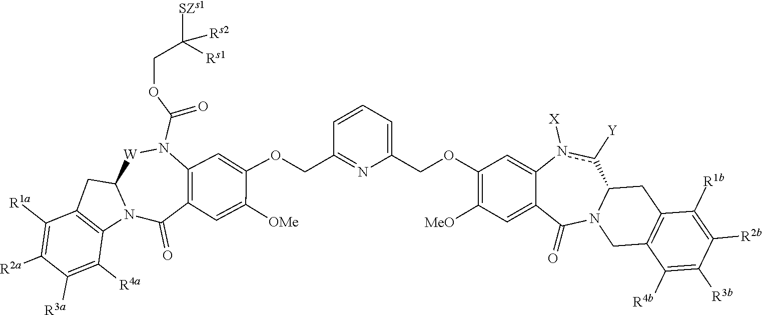 | (TC1dD) |
| 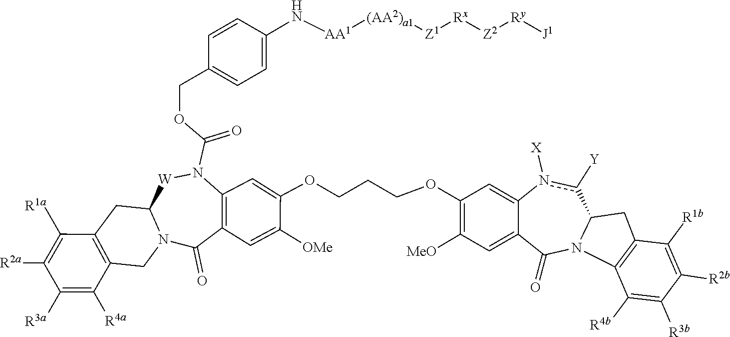 | (TC2aA) |
| 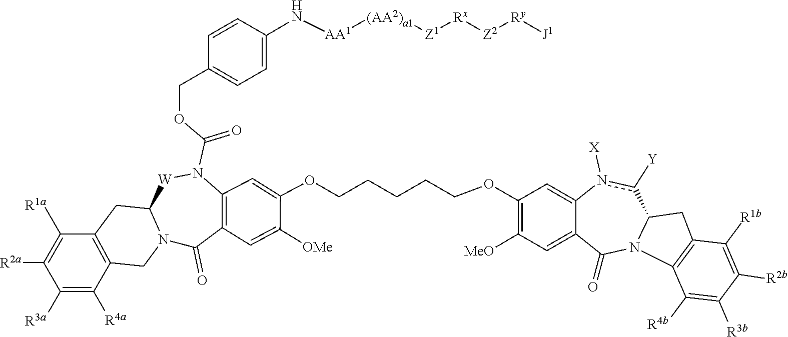 | (TC2aB) |
| 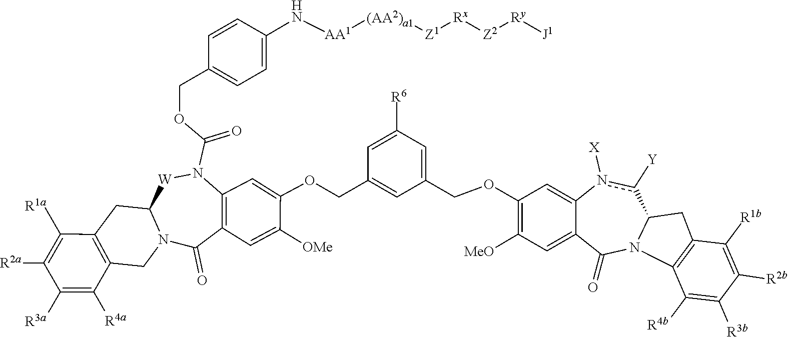 | (TC2aC) |
| 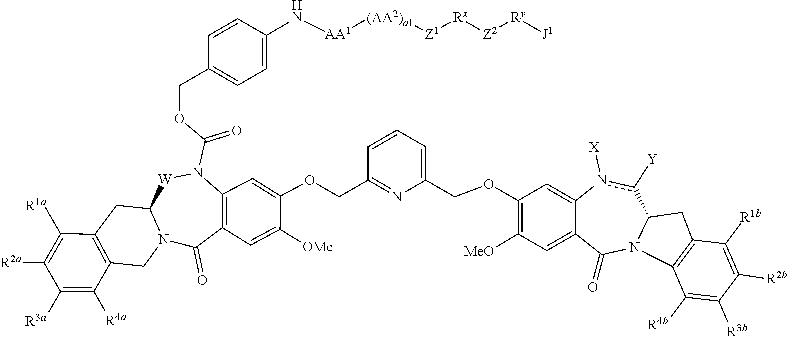 | (TC2aD) |
| 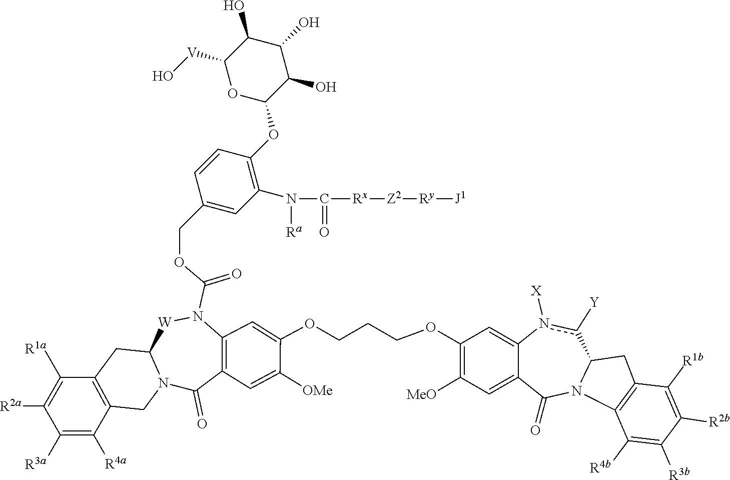 | (TC2bA) |
| 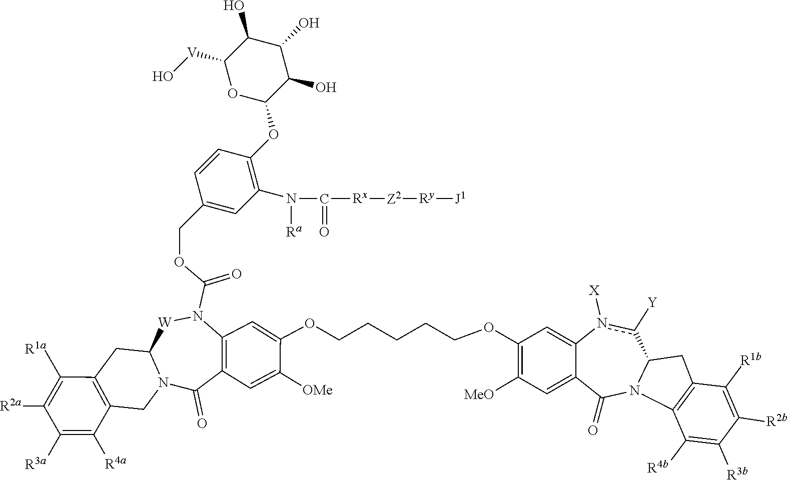 | (TC2bB) |
| 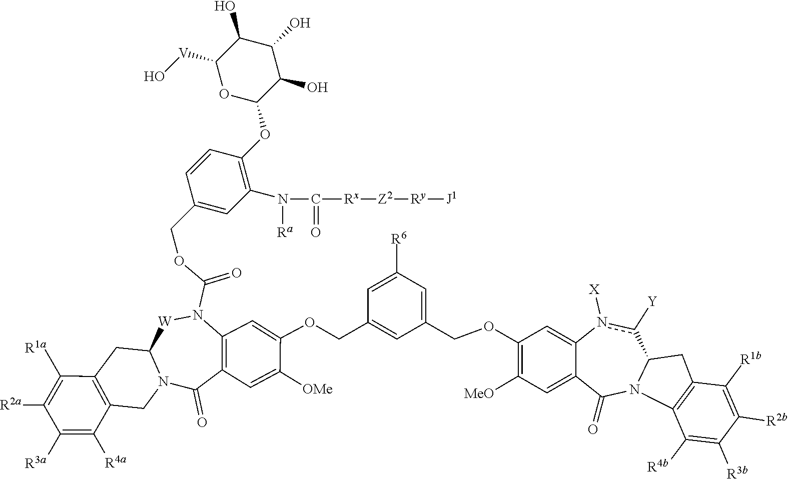 | (TC2bC) |
| 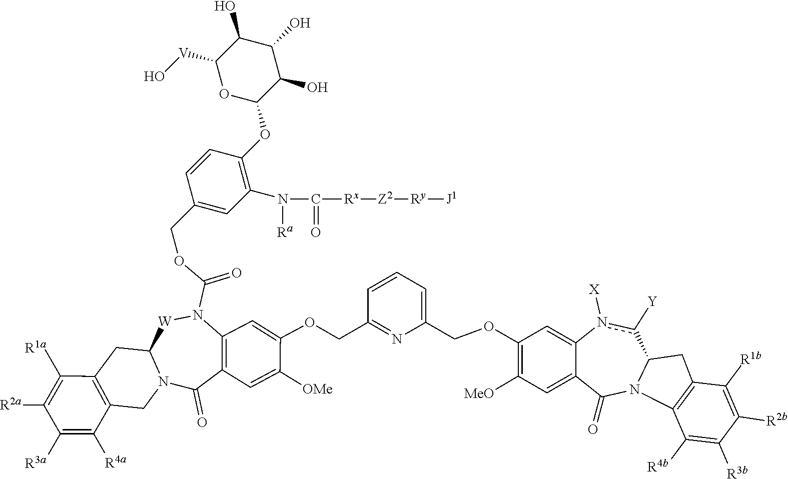 | (TC2bD) |
| 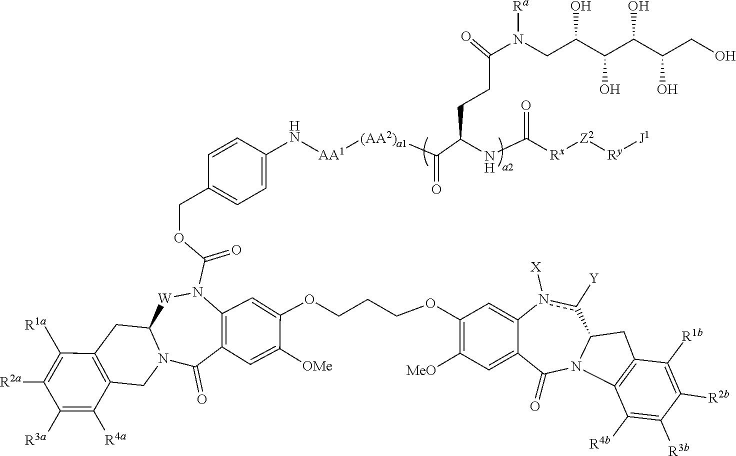 | (TC2cA) |
| 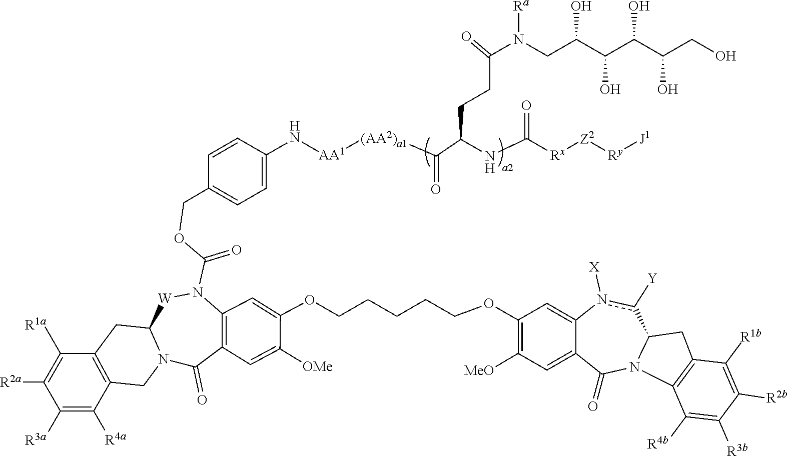 | (TC2cB) |
| 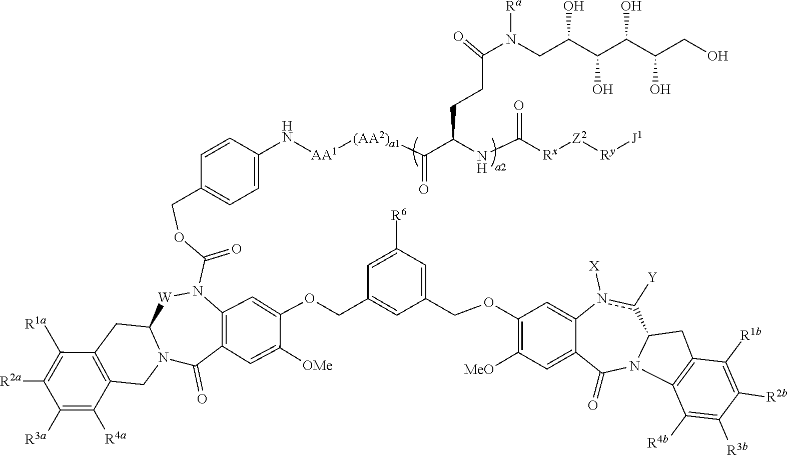 | (TC2cC) |
| 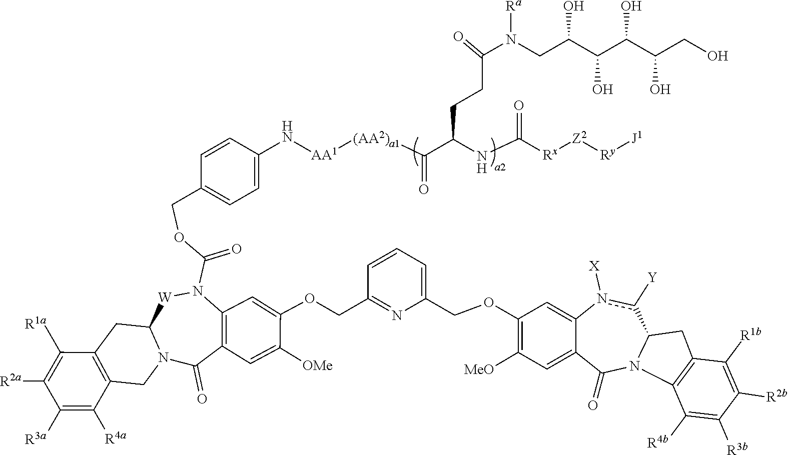 | (TC2cD) |
| 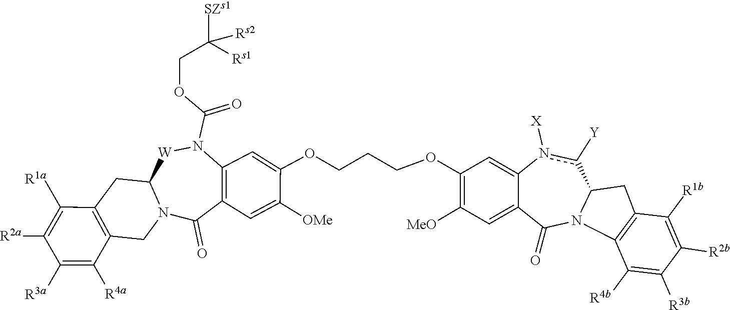 | (TC2dA) |
| 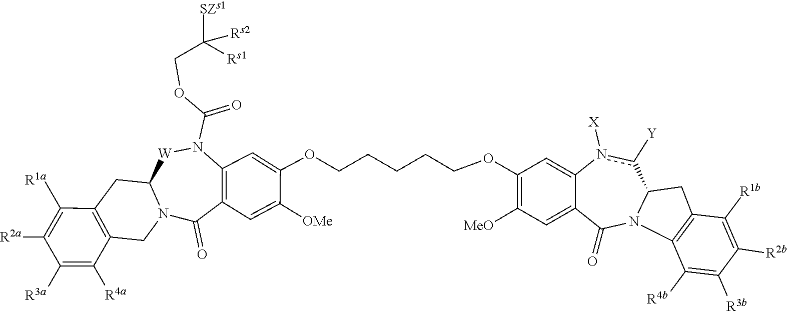 | (TC2dB) |
| 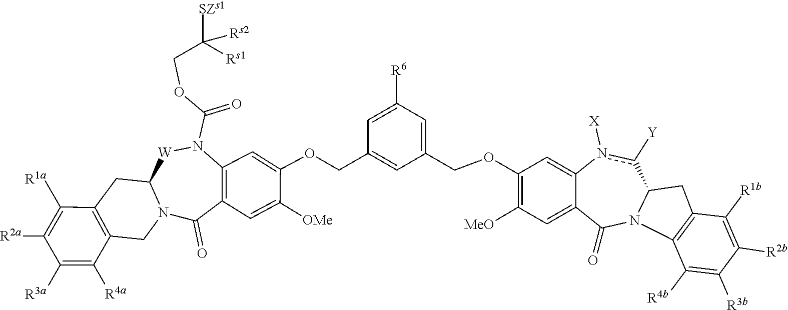 | (TC2dC) |
| 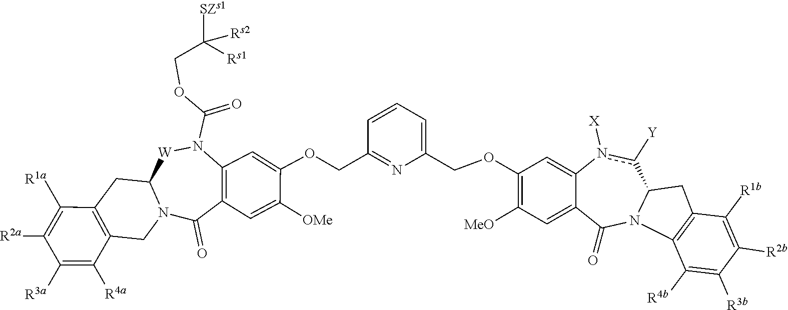 | (TC2dD) |

or a pharmaceutically acceptable salt thereof, wherein the remaining variables are as defined in the second aspect or the 23^{rd} embodiment or any specific embodiment described therein.

In a 28^{th} embodiment, for conjugates described in the 23^{rd} to 27^{th} embodiments, or a pharmaceutically acceptable salt thereof, Z¹ is —C(═O)—; and the remaining variables are as defined in the second aspect or the 23^{rd}, 24^{th}, 25^{th}, 26^{th}, or 27^{th} embodiment or any specific embodiments described therein.

In a 29^{th} embodiment, for conjugates described in the 23^{rd} to 28^{th} embodiments, or a pharmaceutically acceptable salt thereof, R^{x} is C₁₋₆alkylene; Z² and R^{y} are both absent; and the remaining variables are as defined in the second aspect or the 23^{rd}, 24^{th}, 25^{th}, 26^{th}, 27^{th}, or 28^{th} embodiments or any specific embodiment described therein. In another embodiment, R^{x}, Z² and R^{y} are absent; and the remaining variables are as defined in the 23^{rd}, 24^{th}, 25^{th}, 26^{th}, 27^{th}, or 28^{th} embodiment or any specific embodiment described therein.

In a 30^{th} embodiment, for conjugates described in the 23^{rd} to 28^{th} embodiments, or a pharmaceutically acceptable salt thereof, R^{x} is —(CH₂CH₂O)ₘ₁—C₁₋₆alkylene-; Z² is —NH— C(═O)— or —C(═O)—NH—; R^{y} is C₁₋₆alkylene; and the remaining variables are as defined in the second aspect or the 23^{rd}, 24^{th}, 25^{th}, 26^{th}, 27^{th}, or 28^{th} embodiment or any specific embodiments described therein.

In a 31^{th} embodiment, for conjugates described in the 23^{rd} to 28^{th} embodiments, or a pharmaceutically acceptable salt thereof, R^{x} is C₁₋₆alkylene; Z² is —NH—C(═O)— or —C(═O)—NH—; R^{y} is —(CH₂CH₂O)ₘ₂—C₁₋₆alkylene-; and the remaining variables are as defined in the second aspect or the 23^{rd}, 24^{th}, 25^{th}, 26^{th}, 27^{th}, or 28^{th} embodiment or any specific embodiments described therein.

In a 32^{nd} embodiment, for conjugates of the 27^{th} embodiment, or a pharmaceutically acceptable salt thereof, Cy is represented by one of the following formulae in Table G:

| | |
|---|---|
| 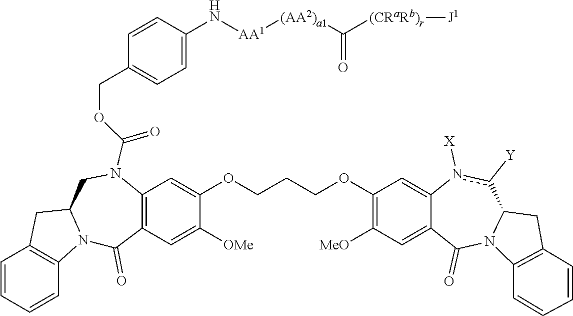 | (VIIa1A) |
| 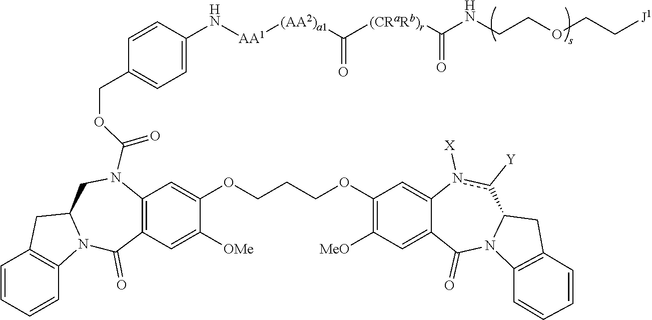 | (VIIa2A) |
| 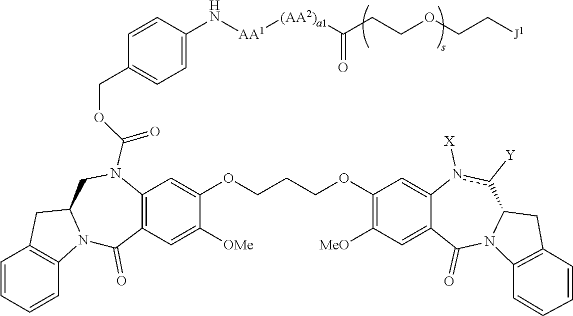 | (VIIa3A) |
| 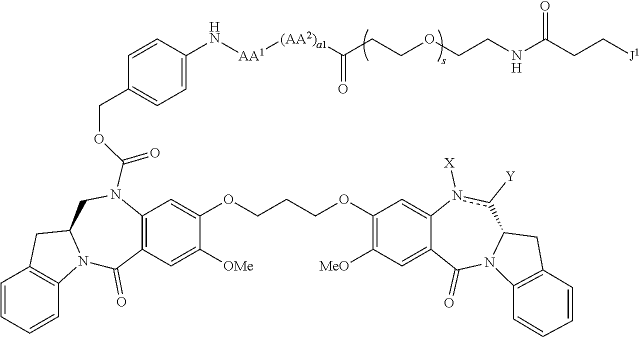 | (VIIa4A) |
| 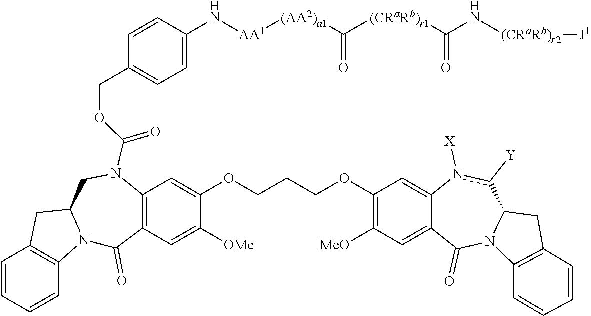 | (VIIa5A) |
| 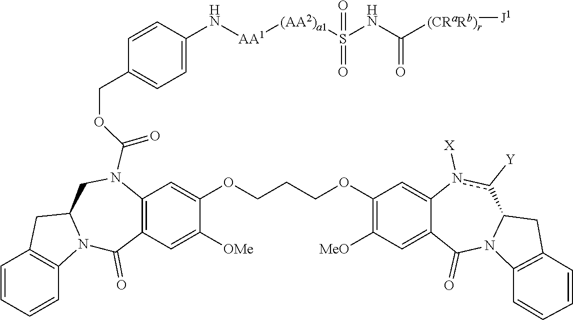 | (VIIa6A) |
| 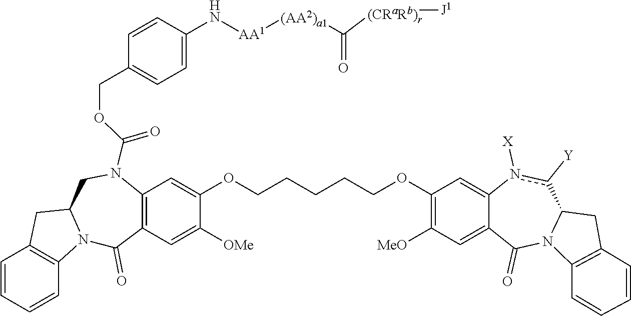 | (VIIa1B) |
| 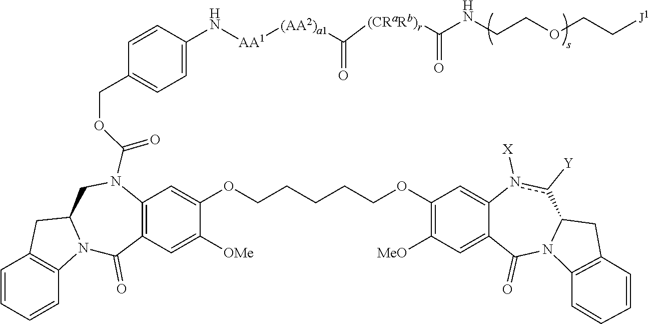 | (VIIa2B) |
| 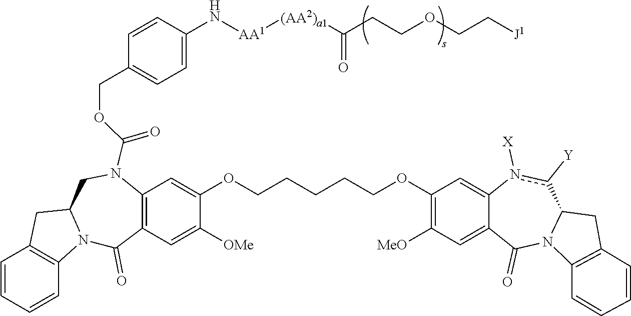 | (VIIa3B) |
| 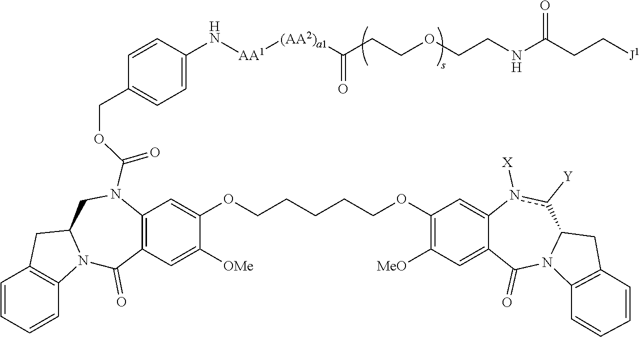 | (VIIa4B) |
| 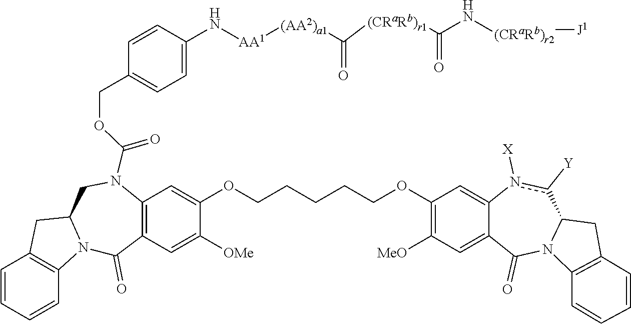 | (VIIa5B) |
| 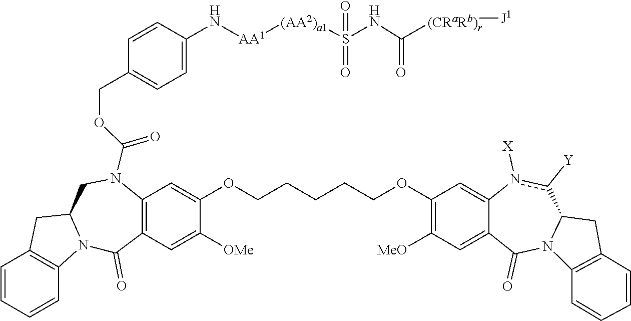 | (VIIa6B) |
| 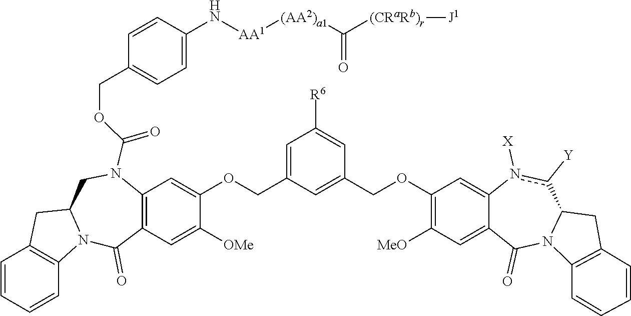 | (VIIa1C) |
| 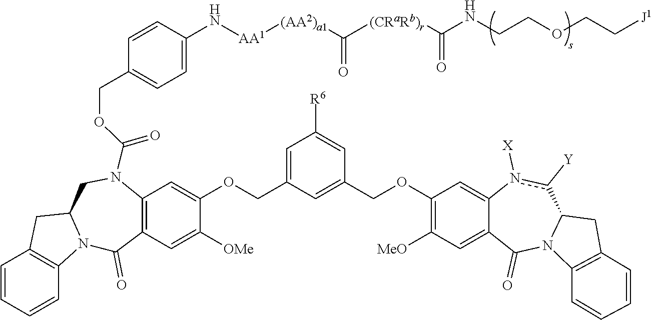 | (VIIa2C) |
| 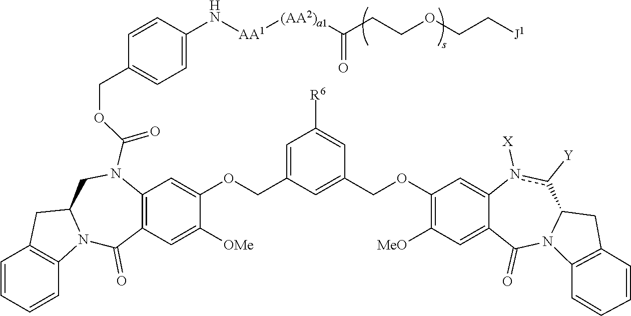 | (VIIa3C) |
| 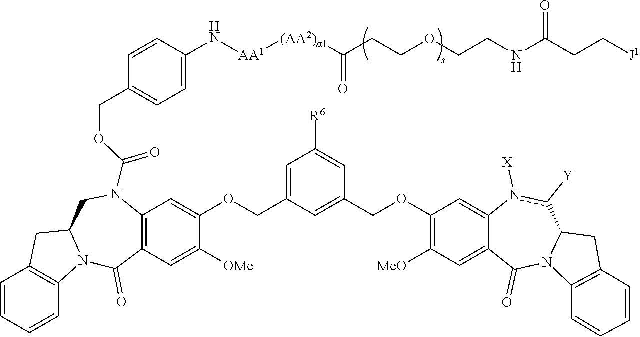 | (VIIa4C) |
| 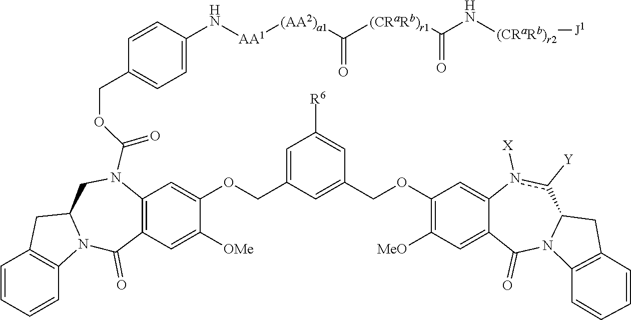 | (VIIa5C) |
| 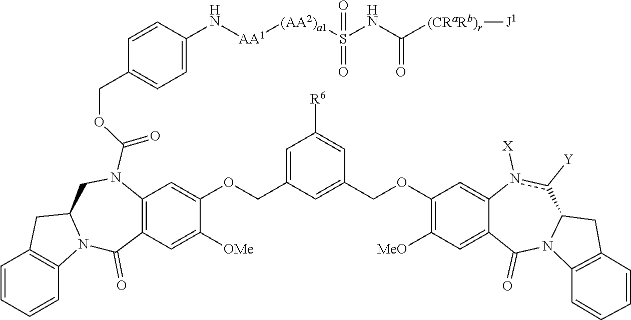 | (VIIa6C) |
| 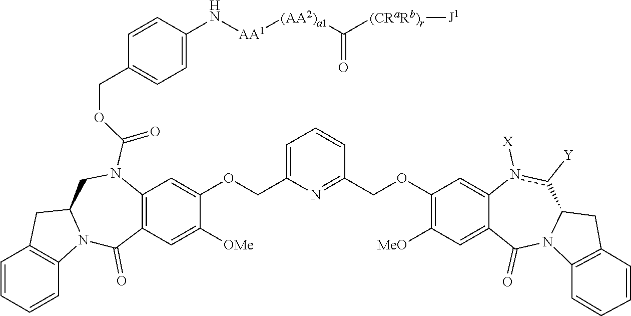 | (VIIa1D) |
| 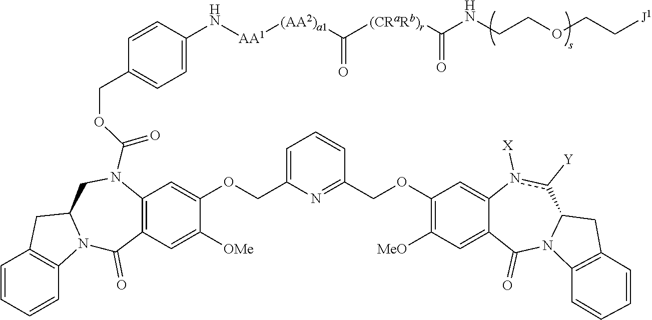 | (VIIa2D) |
| 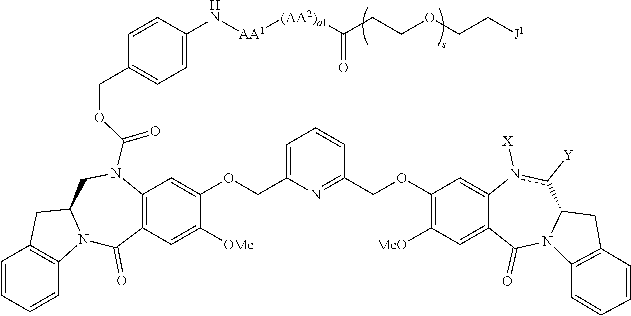 | (VIIa3D) |
| 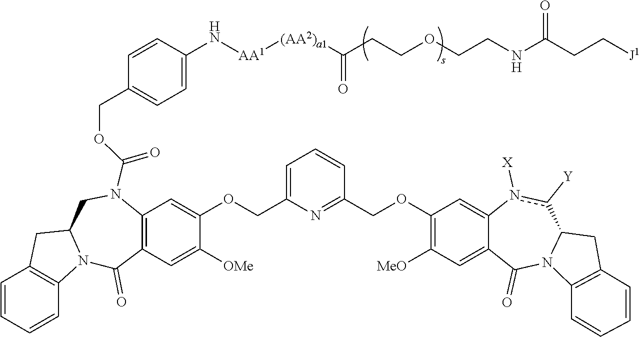 | (VIIa4D) |
| 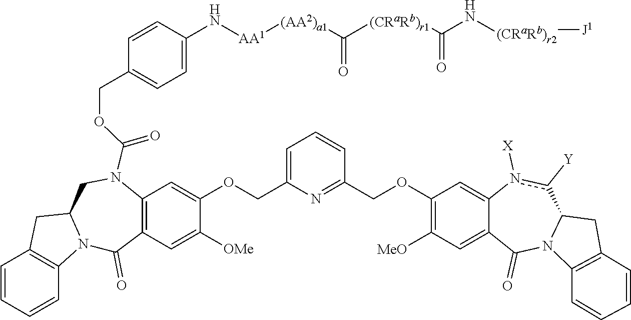 | (VIIa5D) |
| 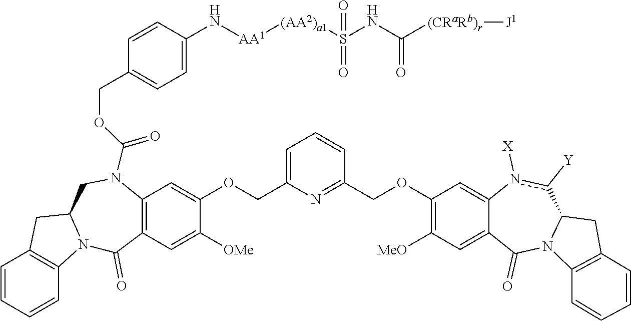 | (VIIa6D) |
| 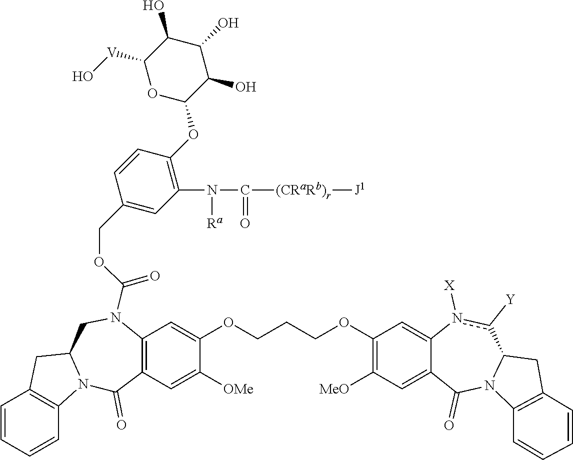 | (VIIb1A) |
| 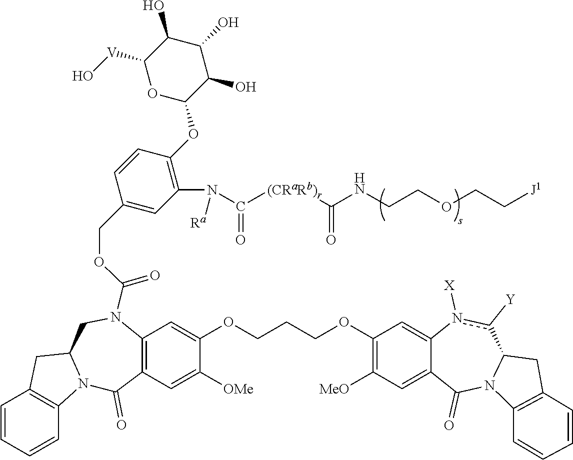 | (VIIb2A) |
| 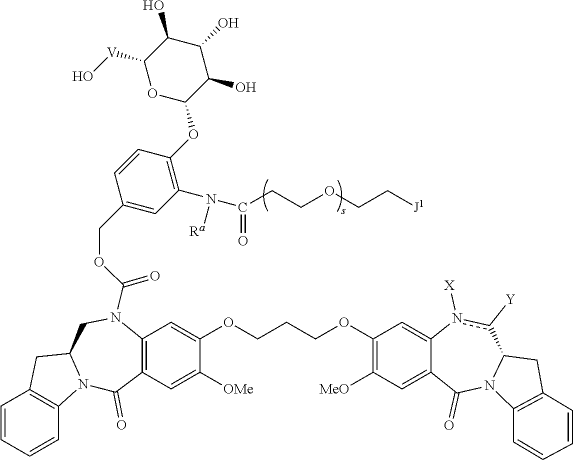 | (VIIb3A) |
| 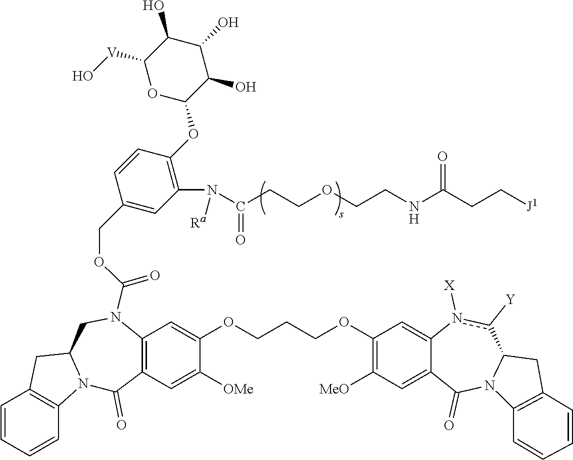 | (VIIb4A) |
| 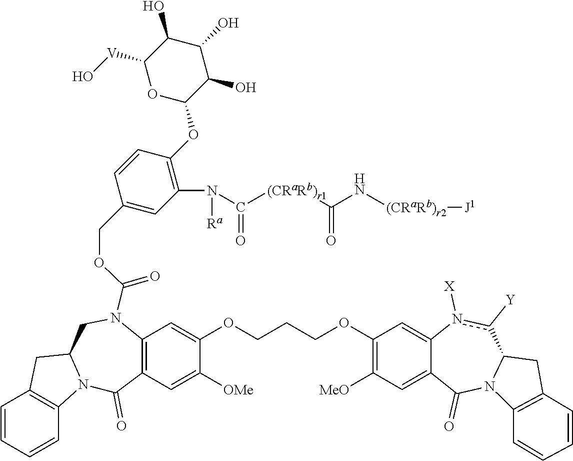 | (VIIb5A) |
| 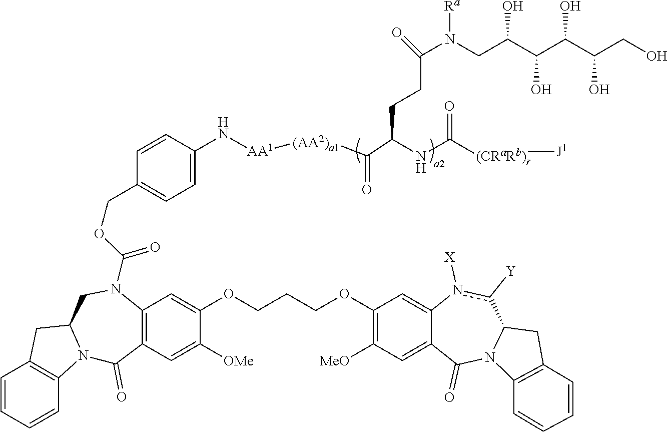 | (VIIc1A) |
| 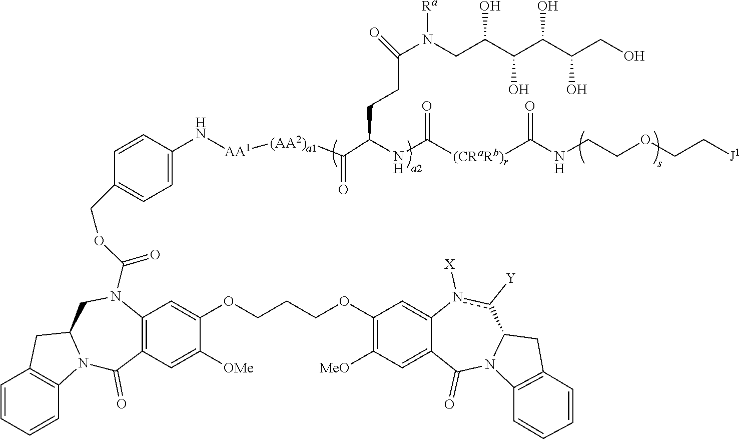 | (VIIc2A) |
| 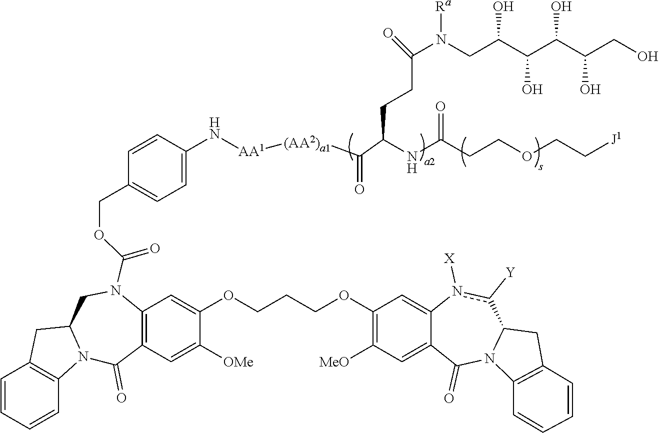 | (VIIc3A) |
| 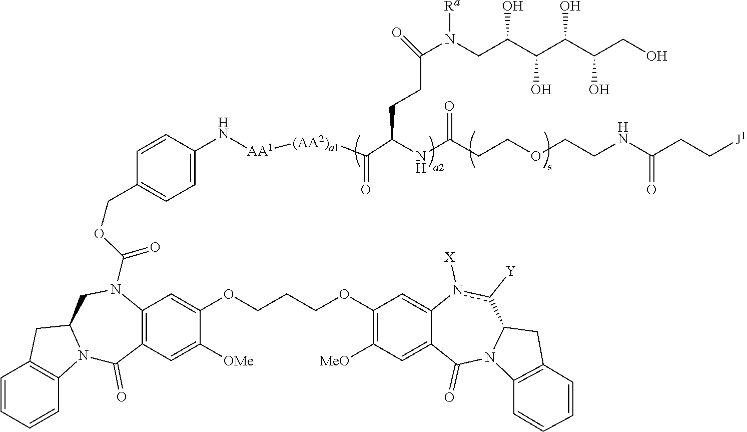 | (VIIc4A) |
| 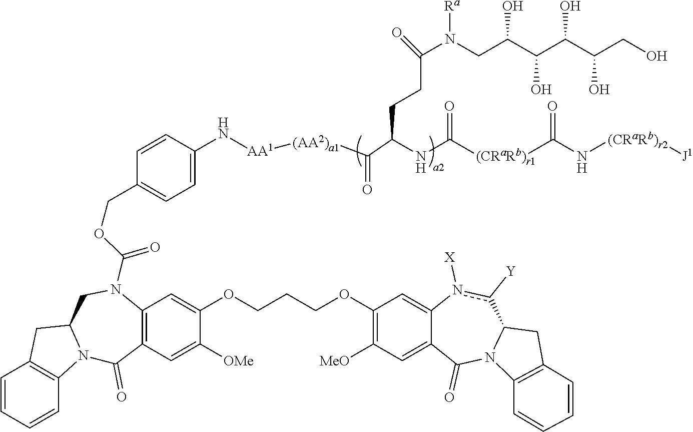 | (VIIc5A) |
| 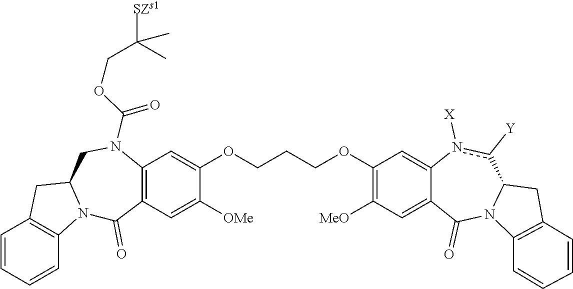 | (VIId1A) |
| 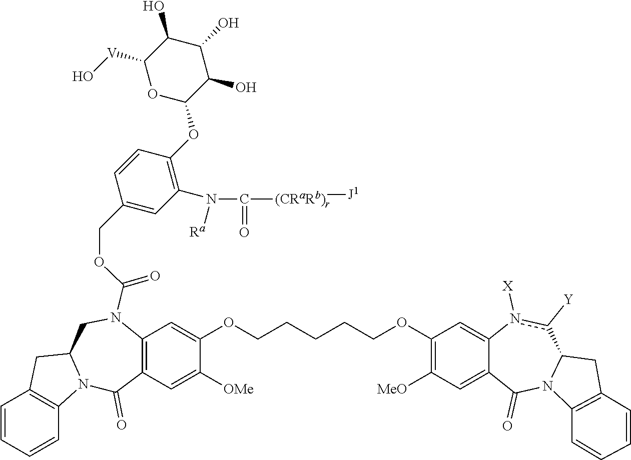 | (VIIb1B) |
| 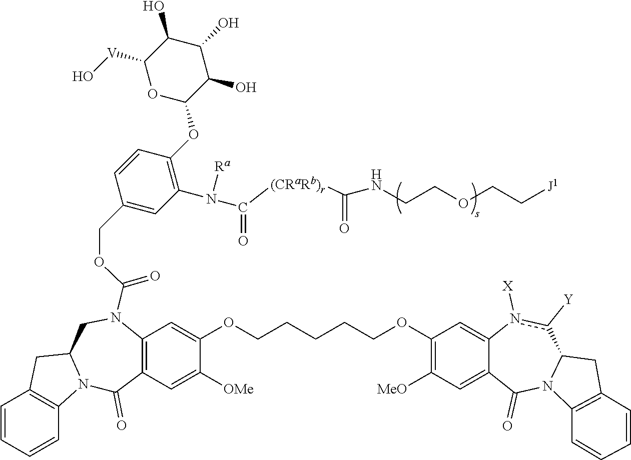 | (VIIb2B) |
| 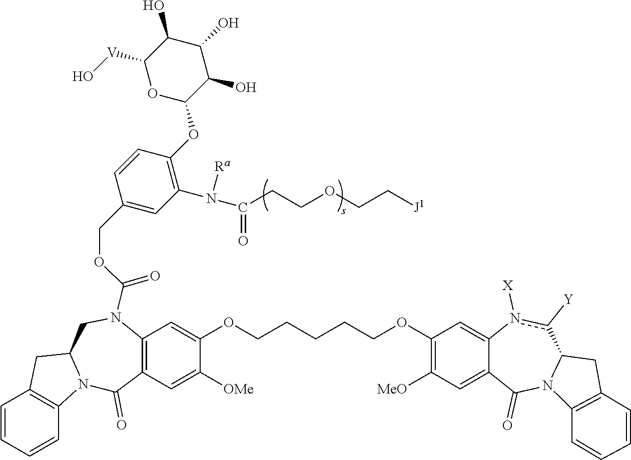 | (VIIb3B) |
| 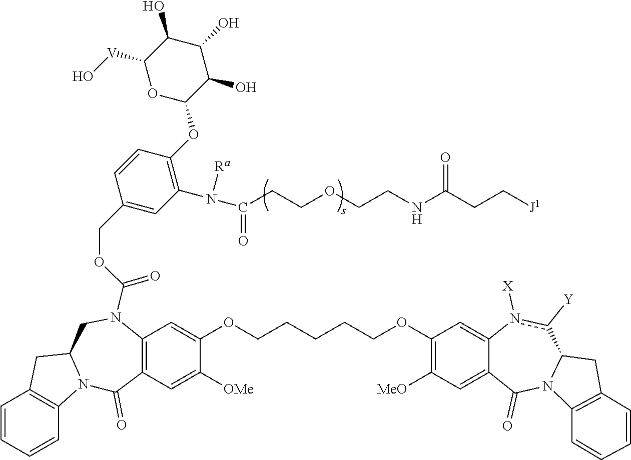 | (VIIb4B) |
| 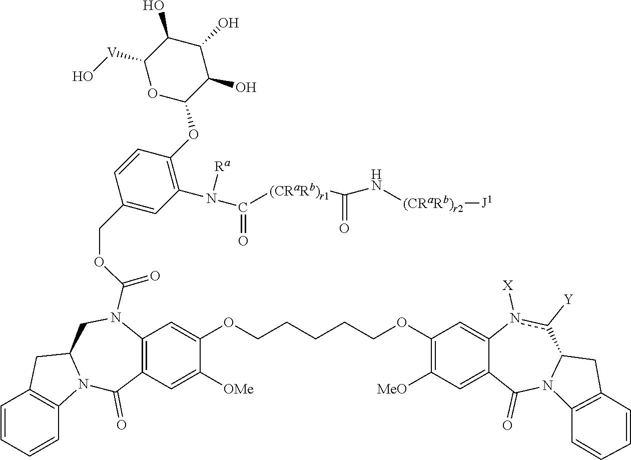 | (VIIb5B) |
| 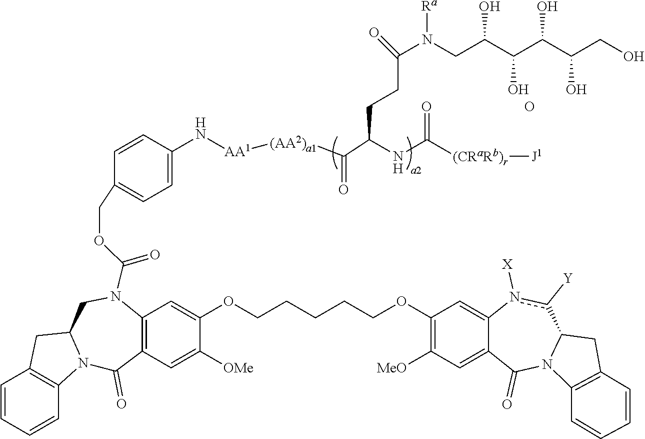 | (VIIc1B) |
| 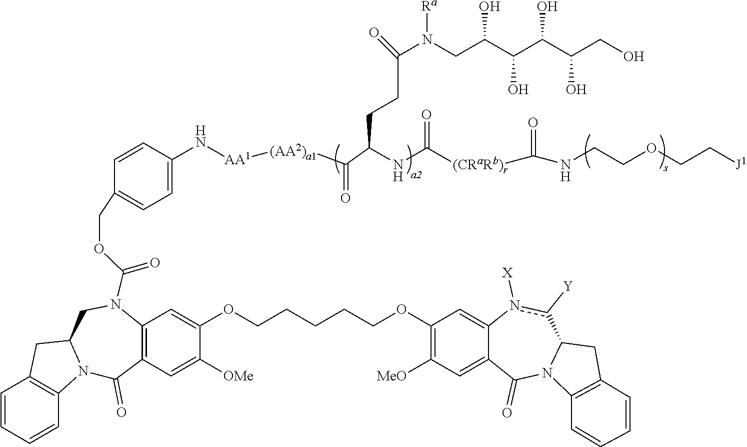 | (VIIc2B) |
| 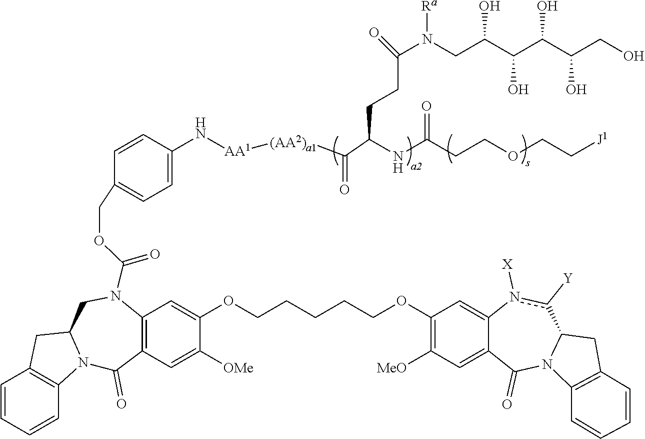 | (VIIc3B) |
| 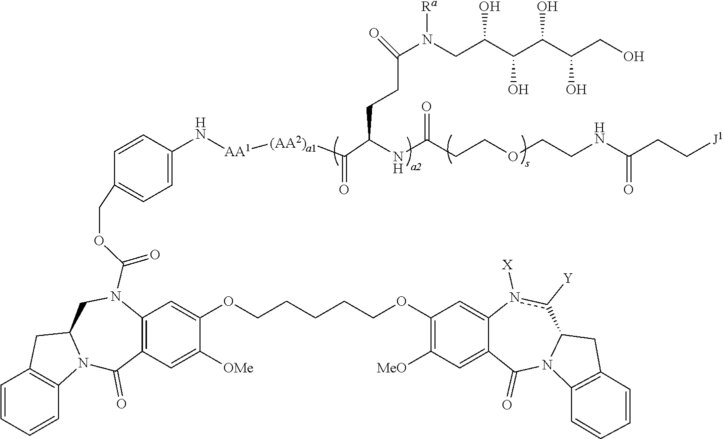 | (VIIc4B) |
| 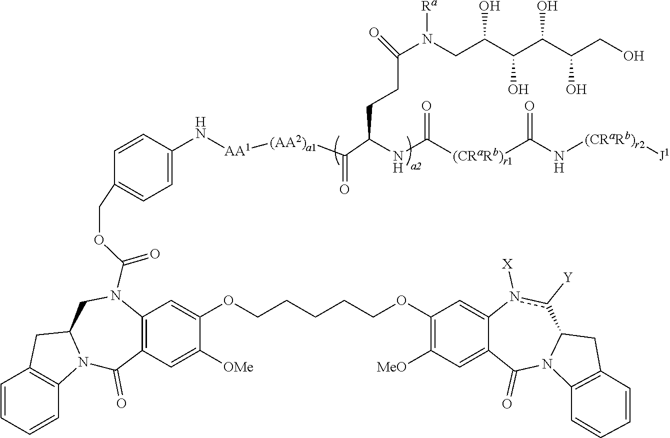 | (VIIc5B) |
| 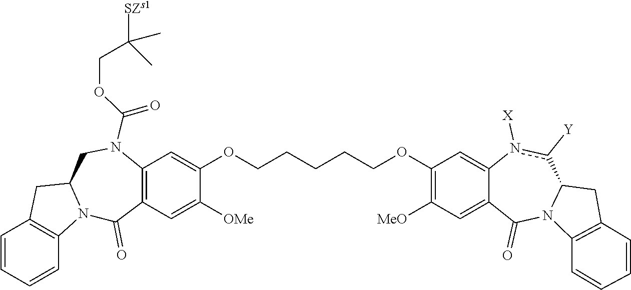 | (VIId1B) |
| 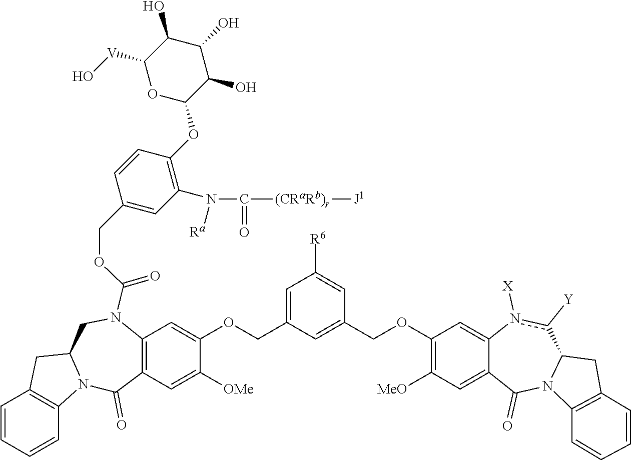 | (VIIb1C) |
| 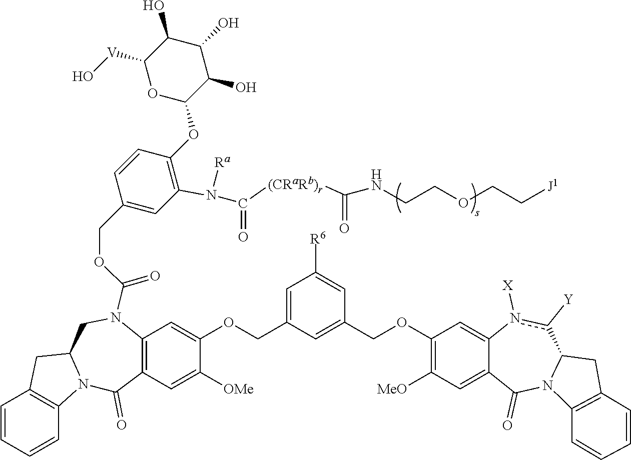 | (VIIb2C) |
| 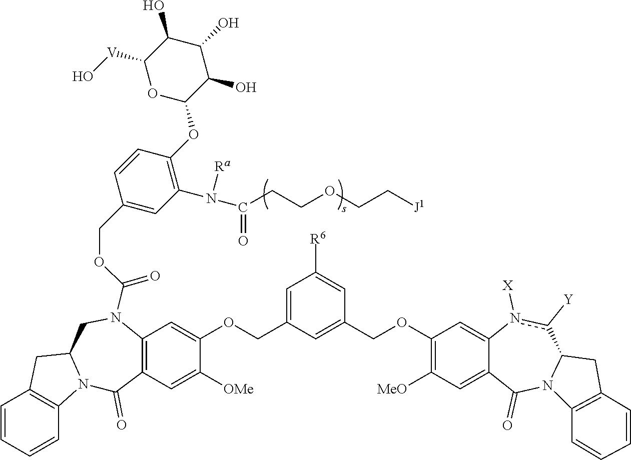 | (VIIb3C) |
| 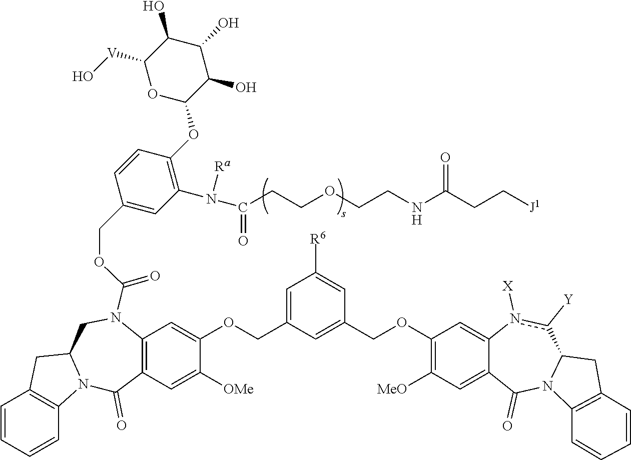 | (VIIb4C) |
| 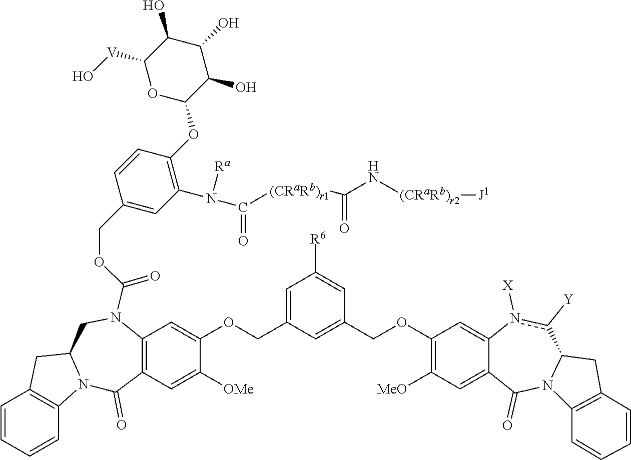 | (VIIb5C) |
| 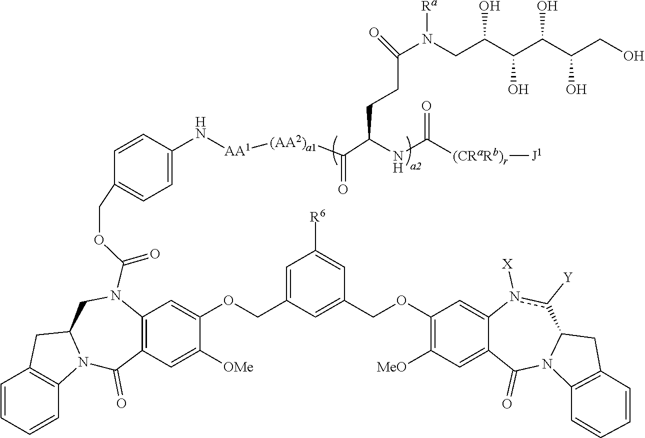 | (VIIc1C) |
| 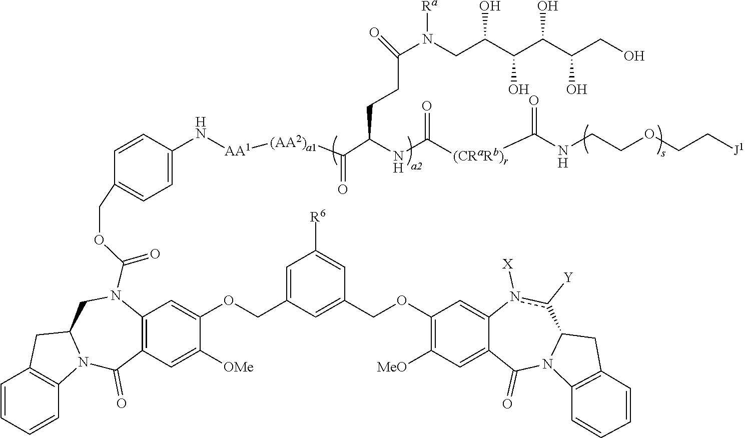 | (VIIc2C) |
| 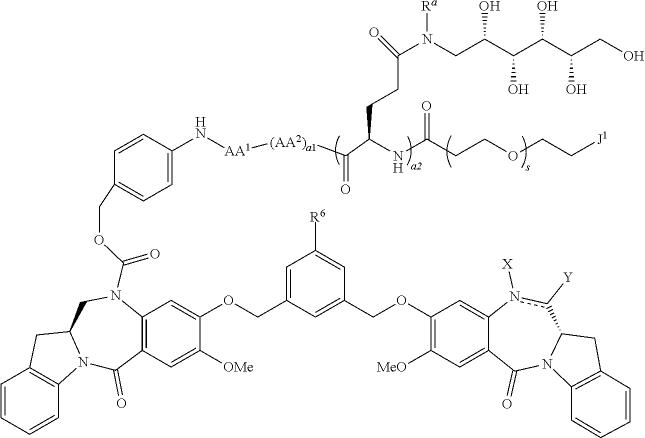 | (VIIc3C) |
| 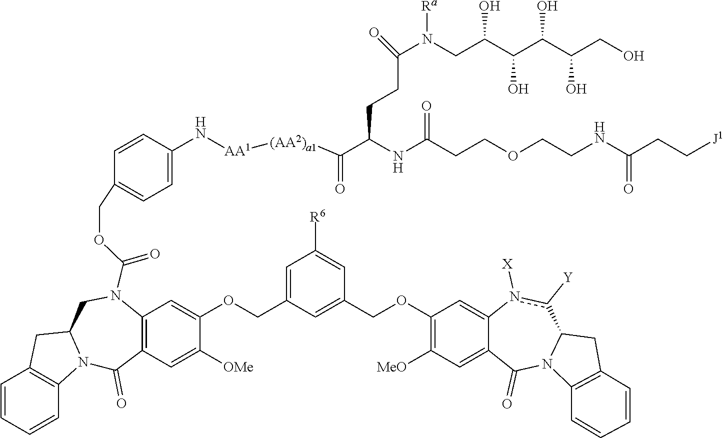 | (VIIc4C) |
| 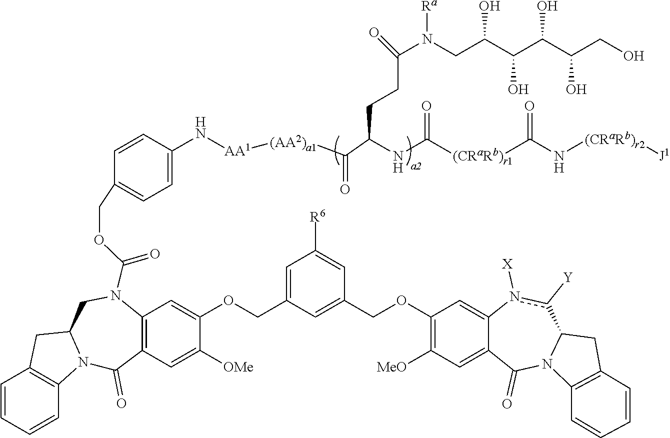 | (VIIc5C) |
| 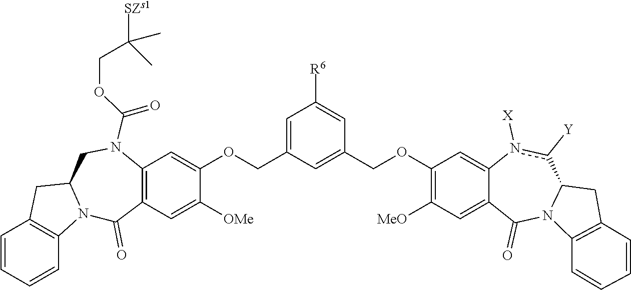 | (VIId1C) |
| 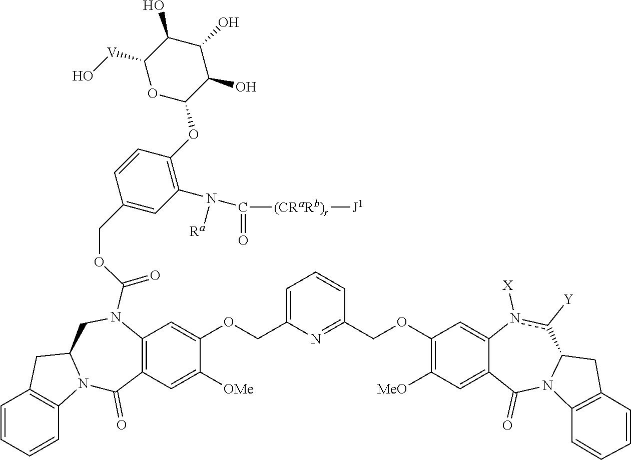 | (VIIb1D) |
| 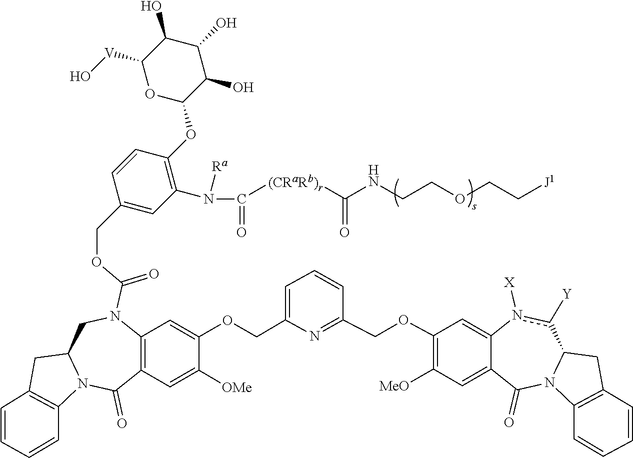 | (VIIb2D) |
| 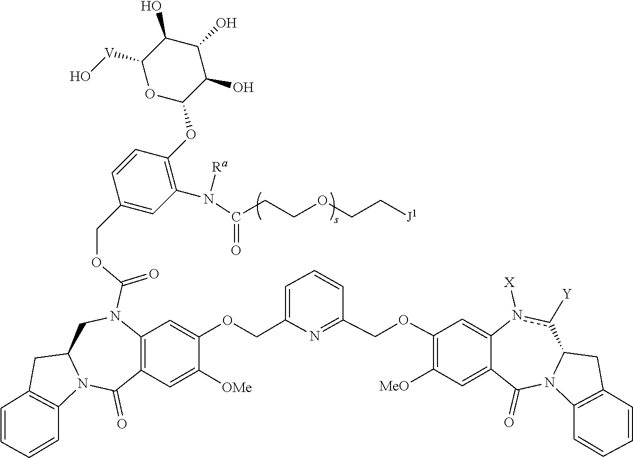 | (VIIb3D) |
| 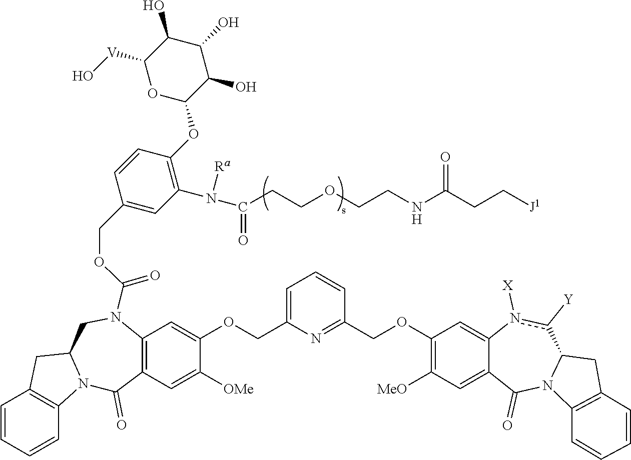 | (VIIb4D) |
| 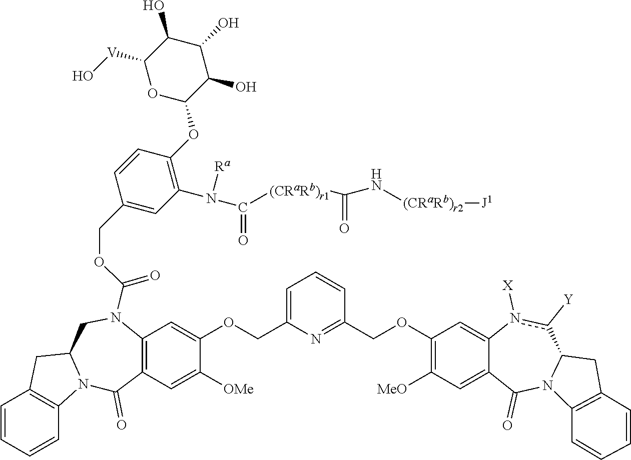 | (VIIb5D) |
| 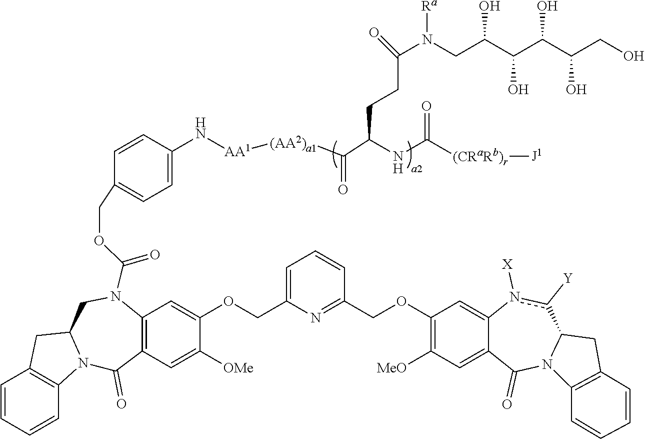 | (VIIc1D) |
| 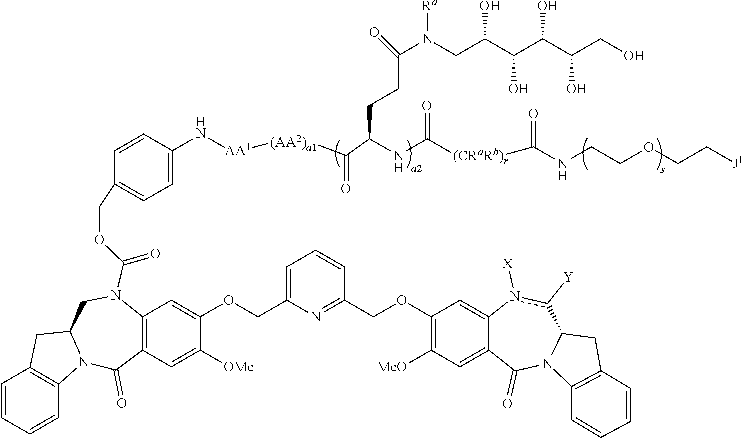 | (VIIc2D) |
| 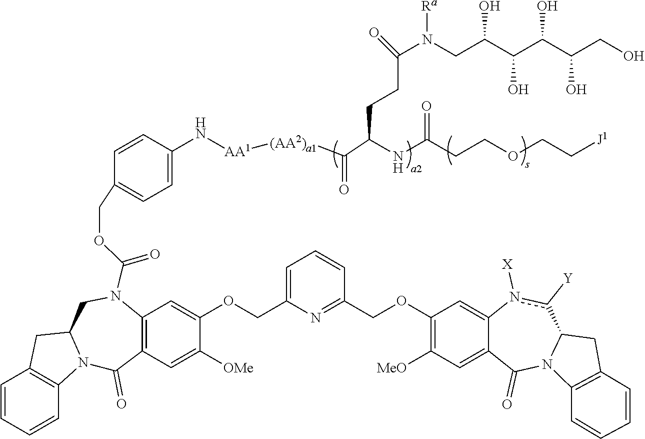 | (VIIc3D) |
| 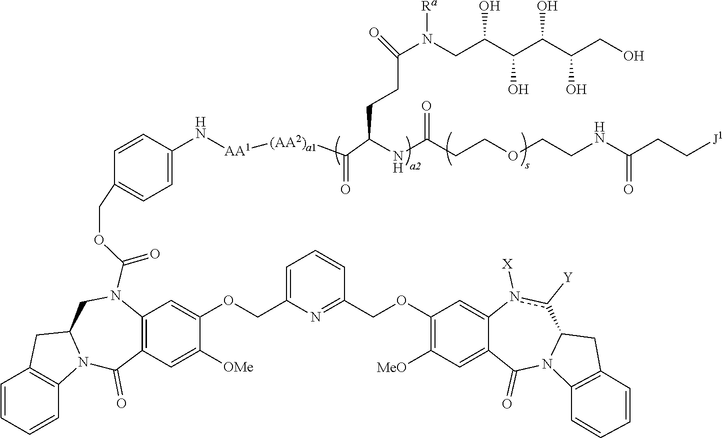 | (VIIc4D) |
| 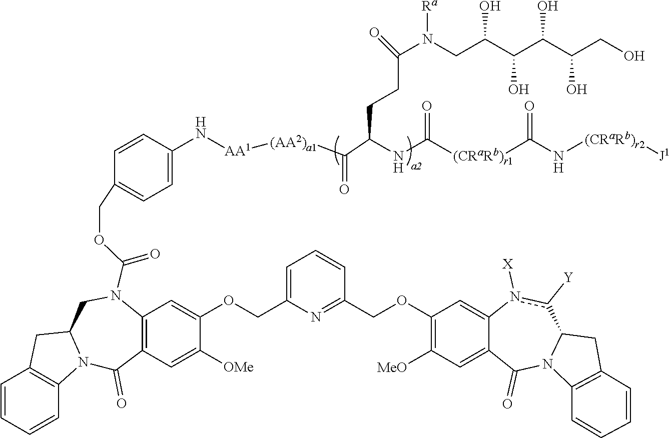 | (VIIc5D) |
| 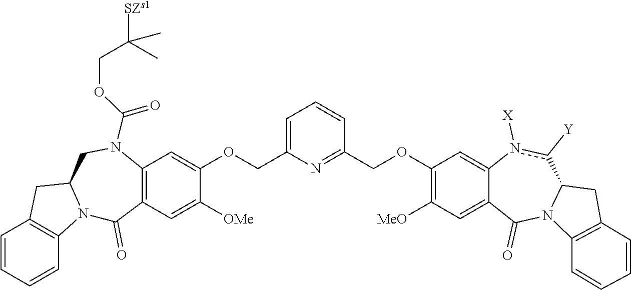 | (VIId1D) |

or a pharmaceutically acceptable salt thereof, wherein:

R⁶ is —C(═O)OR^{6a} or —NR^{6b}(CH₂CH₂O)ₙCH₂CH₂OR^{6c};

R^{6a}, R^{6b} and R^{6c} are each independently H or C₁₋₄alkyl;

n is an integer from 1 to 8;

R^{a} and R^{b}, for each occurrence, are independently H or C₁₋₄alkyl;

r, r1 and r2 are each independently an integer from 2 to 6;

s is an integer from 2 to 12; and

the remaining variables are as defined in the second aspect or the 27^{th} embodiment.

Also included in the 32^{nd} embodiment is a conjugate of the second as aspect, wherein Cy is represented by the following formula:

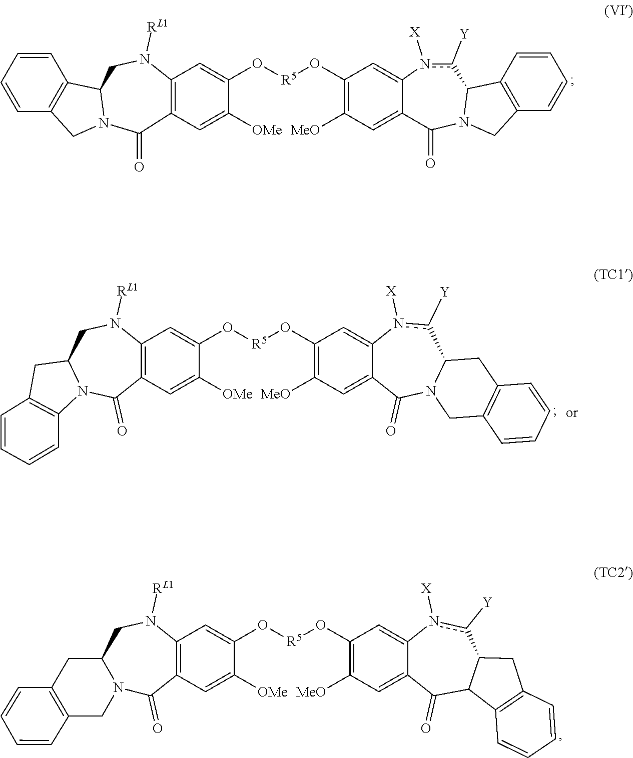

Or a pharmaceutically acceptable salt thereof, wherein:

the double line [symbol] between N and C represents a single bond or a double bond, provided that when it is a double bond X is absent and Y is H, or a C₁₋₄alkyl, and when it is a single bond, X is H and Y is —SO₃H;

R^{L1} is represented by any one of following formula:

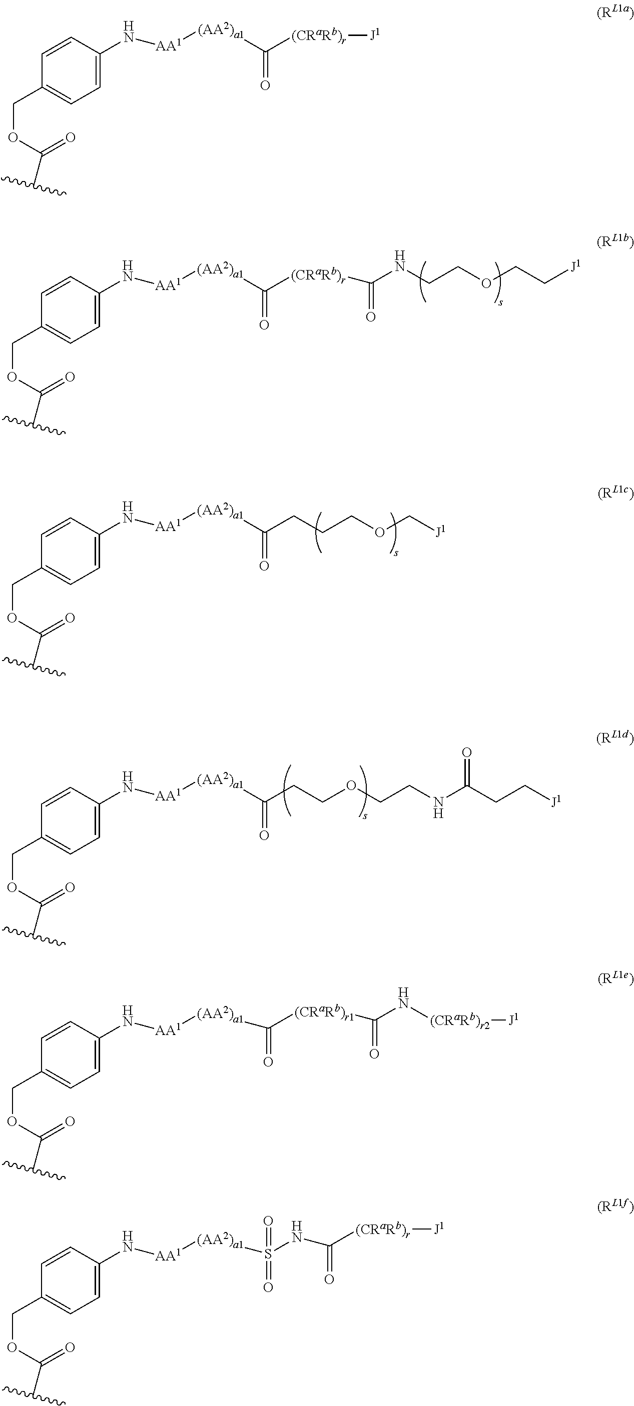

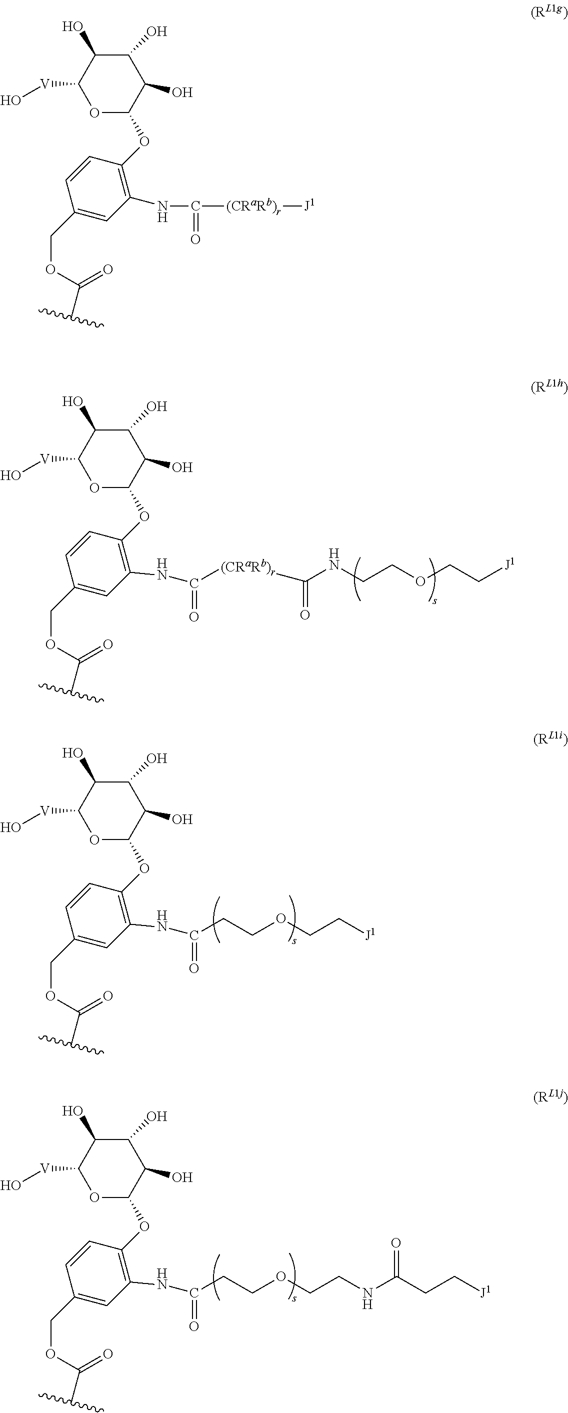

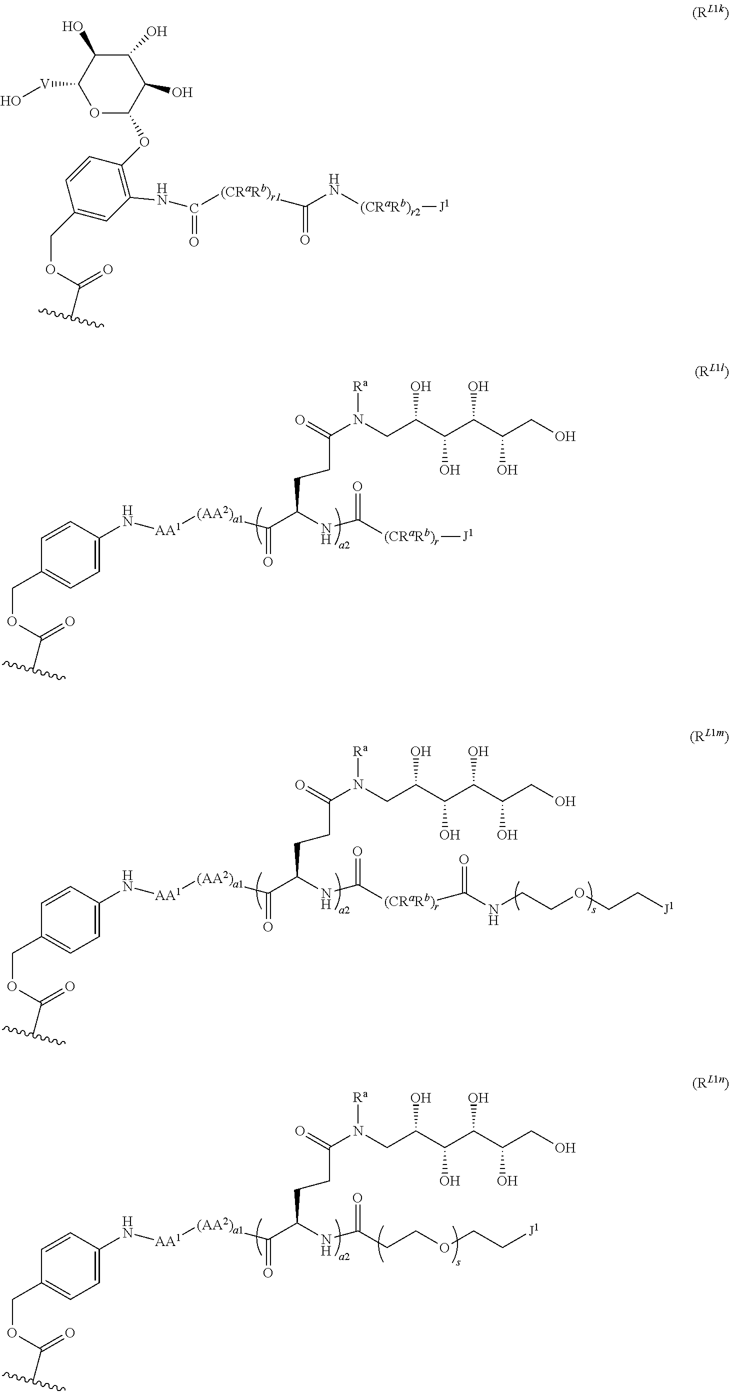

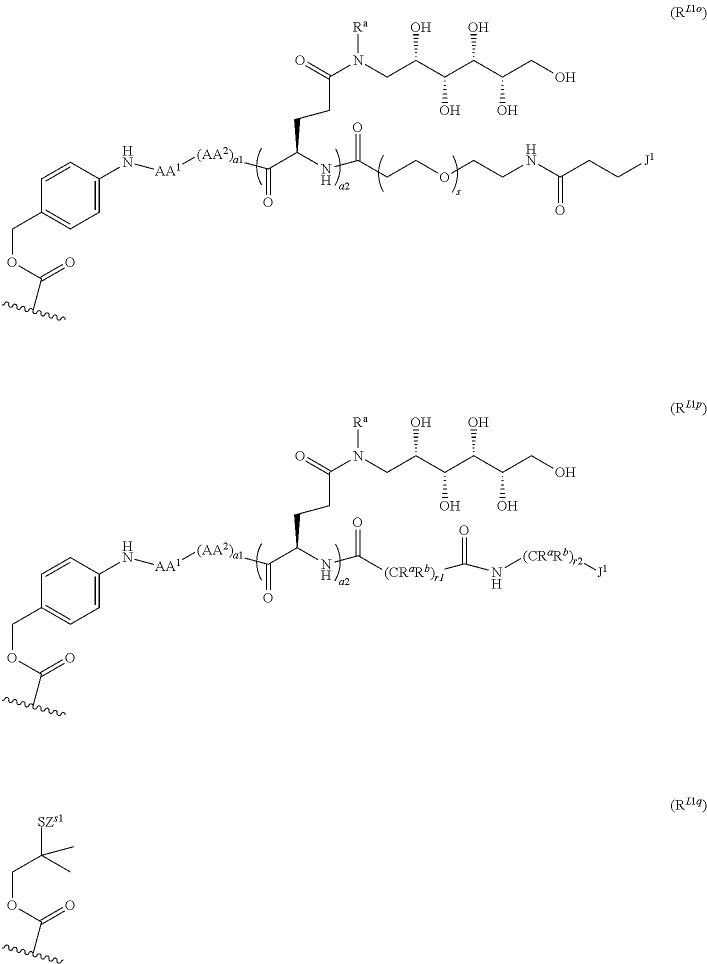

R⁵ is represented by one of the following formulae:

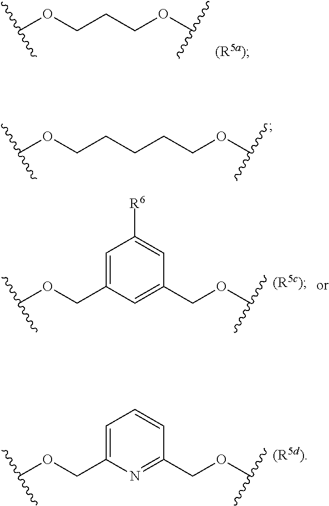

Any combination of R^{L1} and R⁵ are included in the invention.

In a 33^{rd} embodiment, for conjugates of the 32^{nd} embodiment, or a pharmaceutically acceptable salt thereof, wherein:

R^{6a} and R^{6c} are both Me;

R^{6b} is H;

n is 1, 2, 3 or 4;

R^{a} and R^{b}, for each occurrence, are independently H or Me;

r is 4;

r1 is 4;

r2 is 2;

s is 1, 2, 3 or 4; and

the remaining variables are as defined in the second aspect or the 32^{nd} embodiment.

In a 34^{th} embodiment, for conjugates described in the 23^{rd} to 33^{rd} embodiments, or a pharmaceutically acceptable salt thereof, J¹ is —C(═O)—; and the remaining variables are as defined in the second aspect or the 23^{rd}, 24^{th}, 25^{th}, 26^{th}, 27^{th}, 28^{th}, 29^{th}, 30^{th}, 31^{st}, 32^{nd} or 33^{rd} embodiment or any specific embodiment described herein.

In a 35^{th} embodiment, for conjugates described in the 23^{rd} to 33^{rd} embodiments, or a pharmaceutically acceptable salt thereof, J¹ is

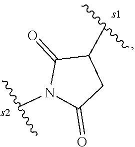

wherein s1 is the site connected to CBA and s2 is the site connected to the rest of the cytotoxic compound; and the remaining variables are as defined in the second aspect or the 23^{rd}, 24^{th}, 25^{th}, 26^{th}, 27^{th}, 28^{th}, 29^{th}, 30^{th}, 31^{st}, 32^{nd} or 33^{rd} embodiment or any specific embodiment described herein.

In a 36^{th} embodiment, for conjugates described in the 23^{rd} to 33^{rd} embodiments, or a pharmaceutically acceptable salt thereof, wherein J¹ is —SZ^{s1}, wherein Z^{s1} is selected from the following formulae:

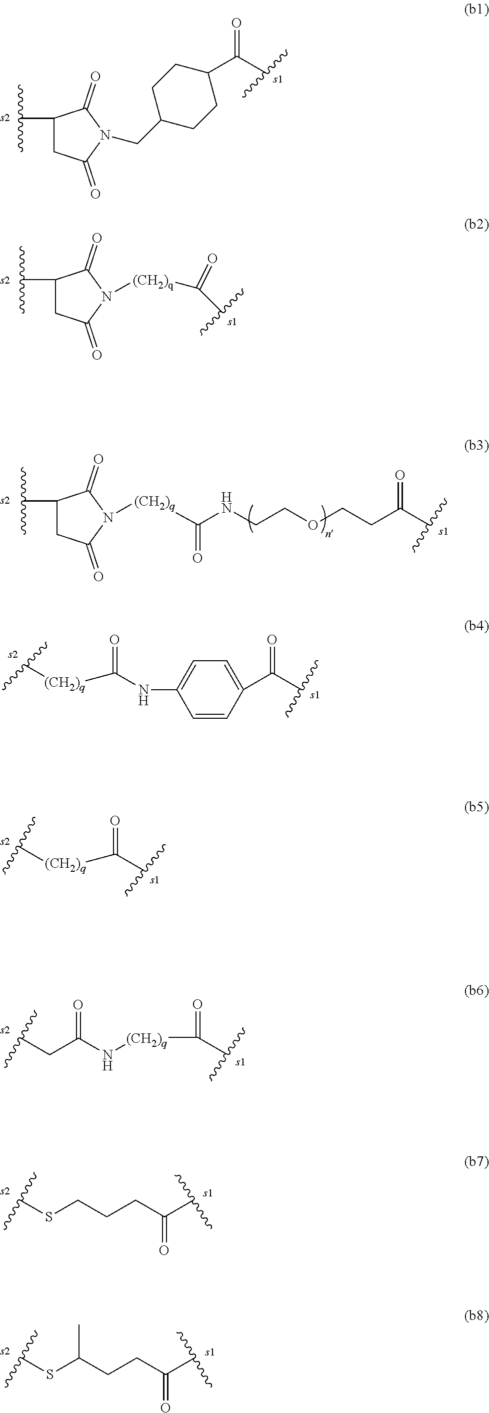

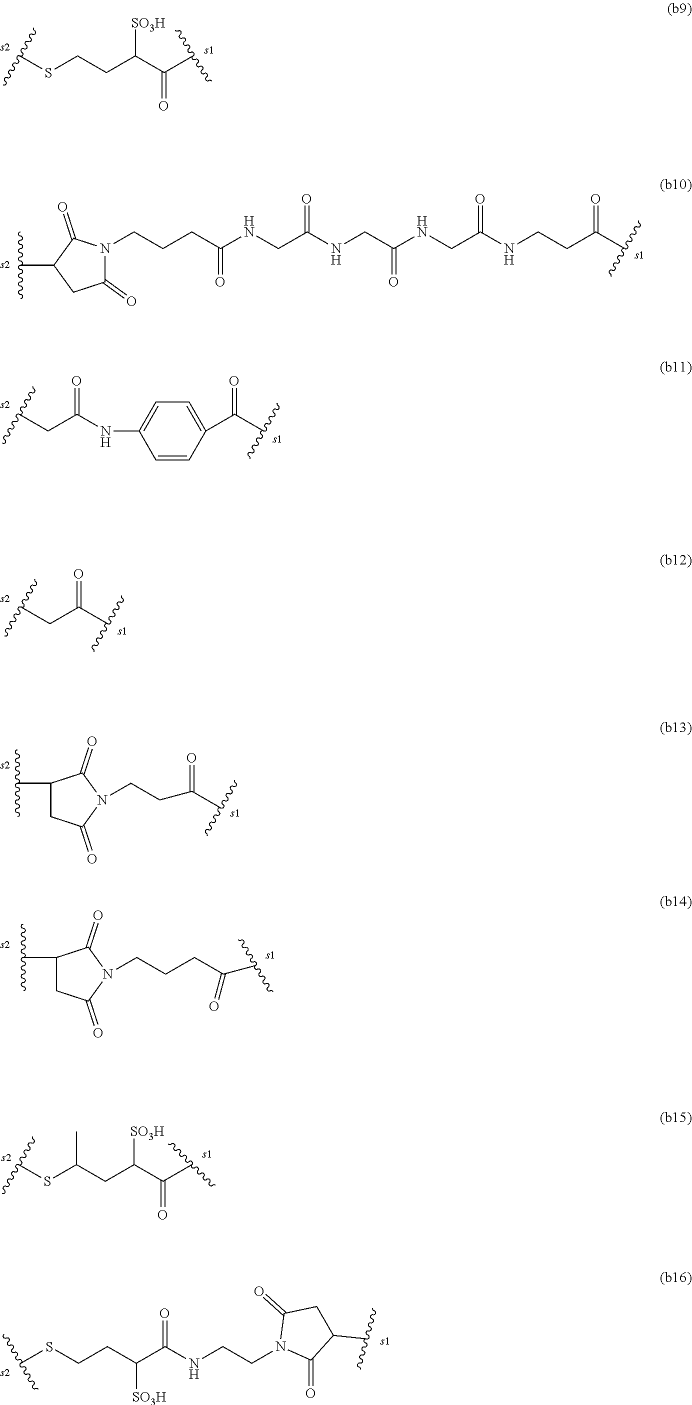

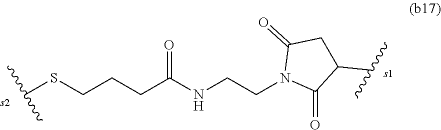

wherein:

q is an integer from 1 to 5;

n′ is an integer from 2 to 6;

s1 is the site connected to CBA;

s2 is the site connected to the rest of the cytotoxic compound; and

the remaining variables are as defined in the second aspect or the 23^{rd}, 24^{th}, 25^{th}, 26^{th}, 27^{th}, 28^{th}, 29^{th}, 30^{th}, 31^{st}, 32^{nd}, or 33^{rd} embodiment or any specific embodiment described herein.

In a specific embodiment, Z^{s1} is represented by formula (b7) or (b9). In another specific embodiment, Z^{s1} is represented by formula (b16) or (b17).

In a 37^{th} embodiment, for conjugates of the 22^{nd} to 36^{th} embodiment, or a pharmaceutically acceptable salt thereof, wherein the double line [symbol] between N and C represents a double bond, X is absent and Y is H; and the remaining variables are as defined in the second aspect or the 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, or 36^{th} embodiment or any specific embodiments described herein.

In a 38^{th} embodiment, for conjugates of the 22^{nd} to 36^{th} embodiment, or a pharmaceutically acceptable salt thereof, the double line [symbol] between N and C represents a single bond, X is H and Y is —SO₃H; and the remaining variables are as defined in the second aspect or the 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, or 36^{th} embodiment or any specific embodiment described herein. In a specific embodiment, the pharmaceutically acceptable salt is a sodium or potassium salt. In another specific embodiment, the pharmaceutically acceptable salt is a sodium salt.

In a 39^{th} embodiment, for conjugates described in the 23^{rd} to 38^{th} embodiments, or a pharmaceutically acceptable salt thereof, wherein a1 is an integer from 1 to 7; and the remaining variables are as defined in the second aspect or the 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, or 38^{th} embodiment or any specific embodiments described herein.

In a specific embodiment, AA¹ and AA² are each independently selected from In a specific embodiment, AA¹ and AA² are each independently selected from Arginine (Arg), Histidine (His), Lysine (Lys), Aspartic acid (Asp), Glutamic Acid (Glu), Serine (Ser), Threonine (Thr), Asparagine (Asn), Glutamine (Gln), Cysteine (Cys), Selenocysteine (Sec), Glycine (Gly), Proline (Pro), Alanine (Ala), Valine (Val), Isoleucine (Ile), Leucine (Leu), Methionine (Met), Phenylalanine (Phe), Tyrosine (Tyr) and Tryptophan (Trp).

In a 40^{th} embodiment, for conjugates of the 39^{th} embodiment, or a pharmaceutically acceptable salt thereof, wherein AA¹-(AA²)ₐ₁ is selected from Gly-Gly-Gly, Ala-Val, Val-Ala, Val-Cit, Val-Lys, Phe-Lys, Lys-Lys, Ala-Lys, Phe-Cit, Leu-Cit, Lle-Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu, β-Ala-Leu-Ala-Leu, Gly-Phe-Leu-Gly, Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Thr-Thr, Thr-Met, Met-Thr, Leu-Ala, Cit-Val, Gln-Val, Ser-Val, Leu-Gln, Gln-Leu, Phe-Arg, Arg-Phe, Tyr-Arg, Arg-Tyr, Phe-Gln, Gln-Phe, Val-Thr, Thr-Val, Met-Tyr, and Tyr-Met; and the remaining variables are as defined in the second aspect or the 39^{th} embodiment.

In a specific embodiment, AA¹-(AA²)ₐ₁ is Ala-Ala, L-Ala-L-Ala, Ala-Val, L-Ala-L-Val, Gln-Val, L-Gln-L-Val, Gln-Leu, L-Gln-L-Leu, Ser-Val, or L-Ser-L-Val.

In a 41^{st} embodiment, the conjugate of the present invention is selected from one of the following in Table H:

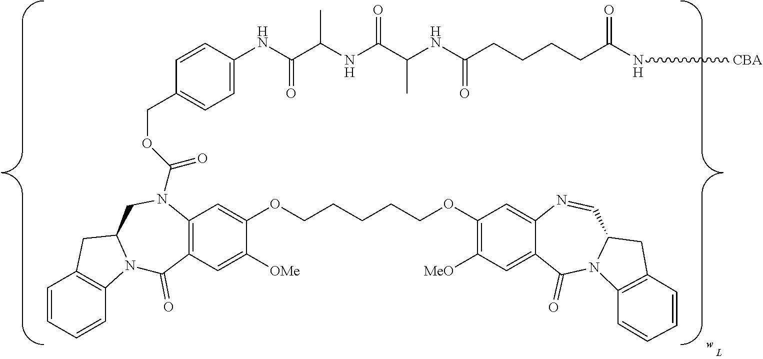

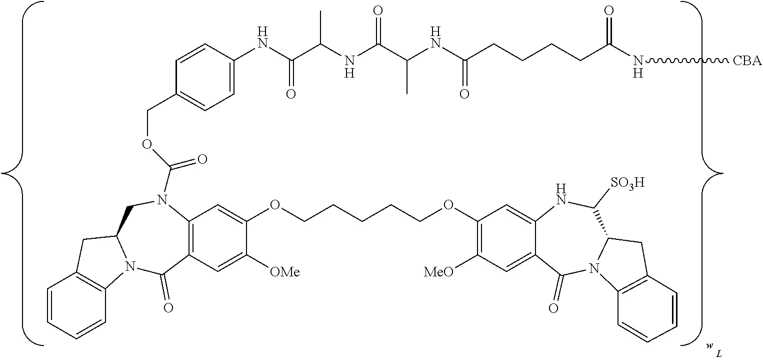

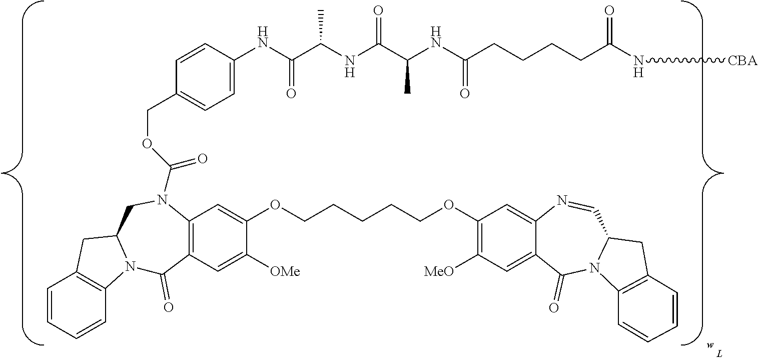

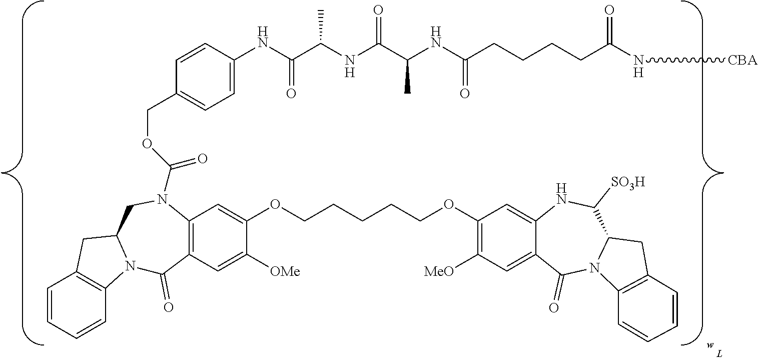

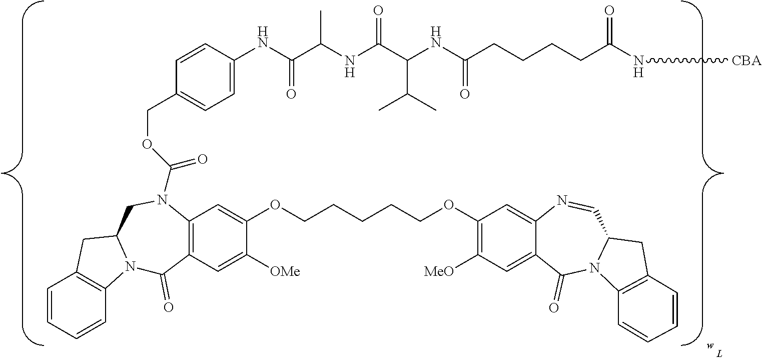

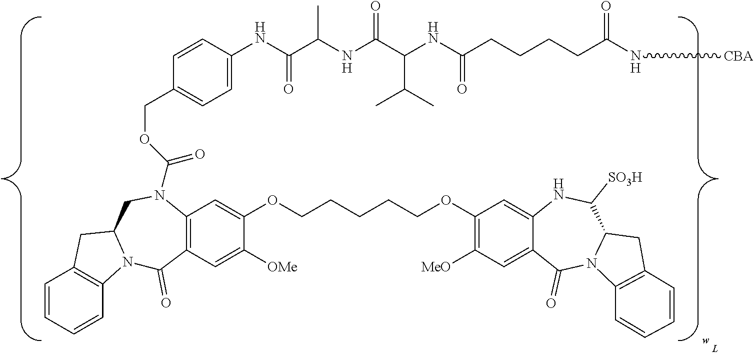

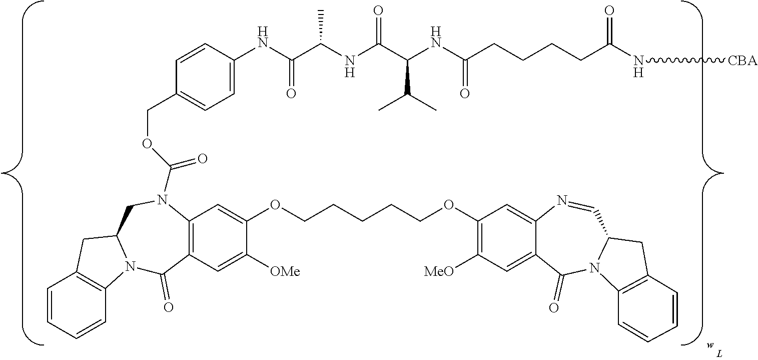

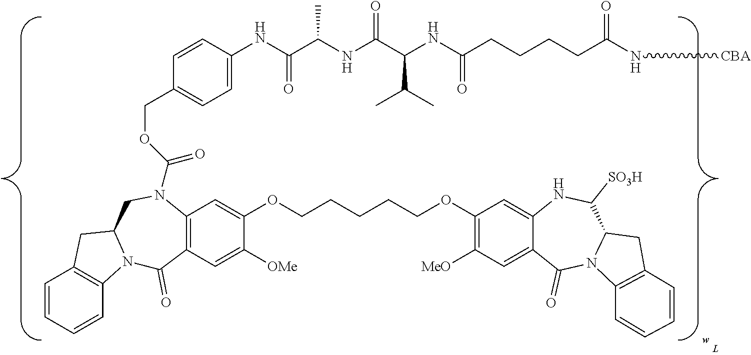

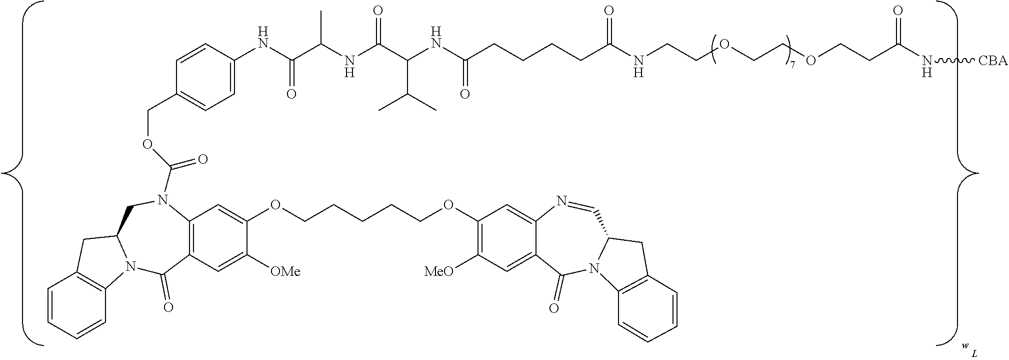

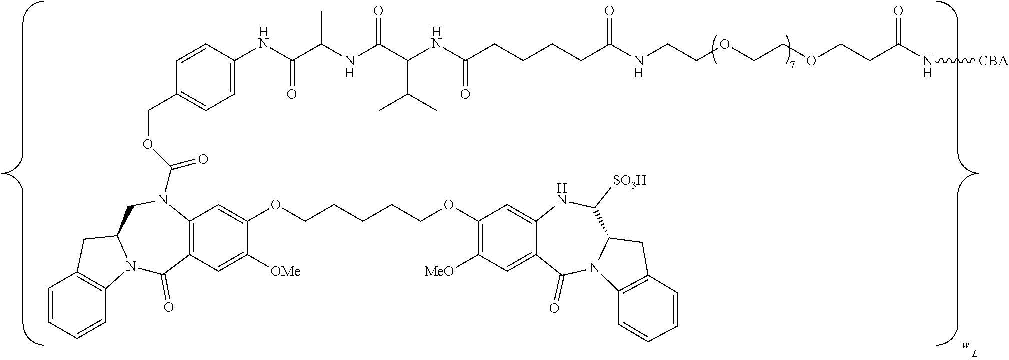

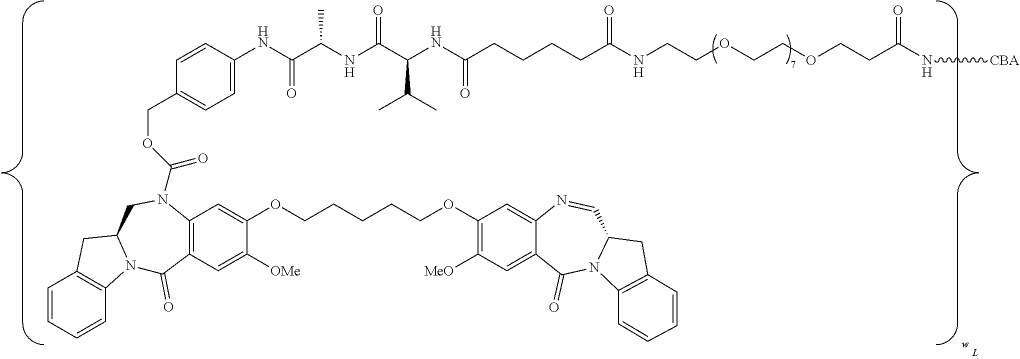

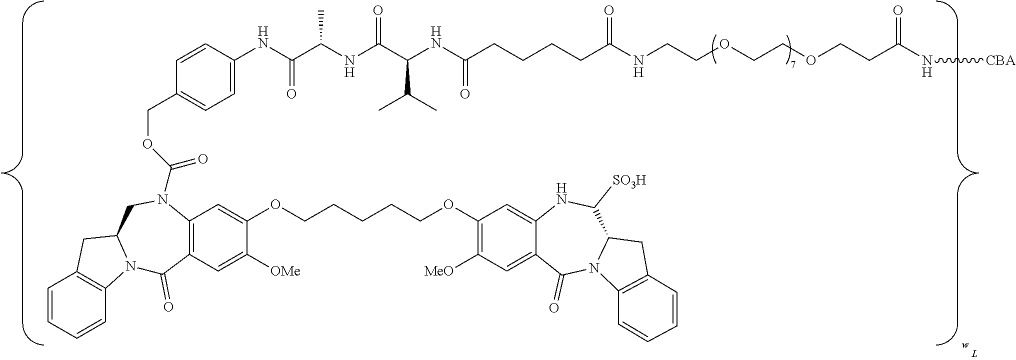

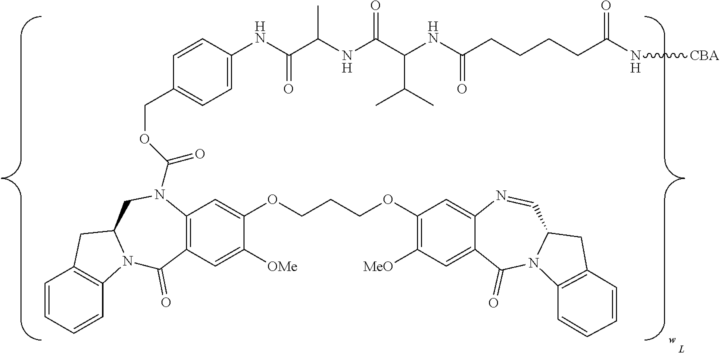

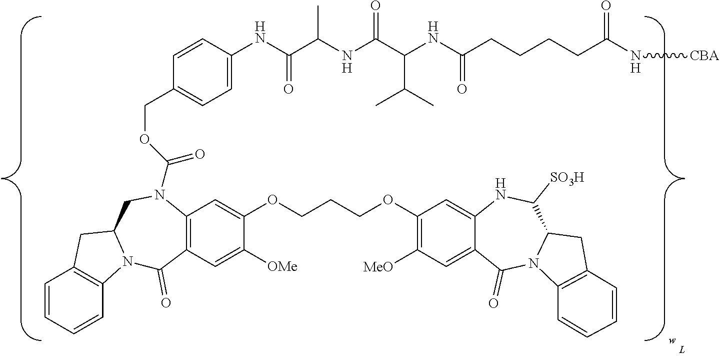

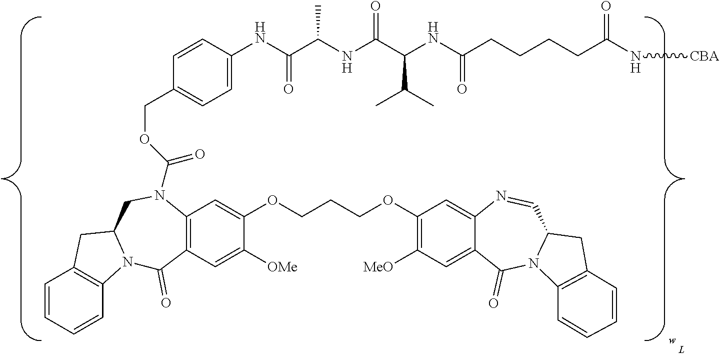

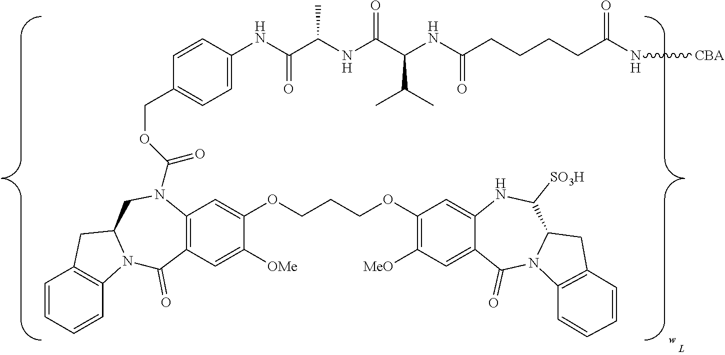

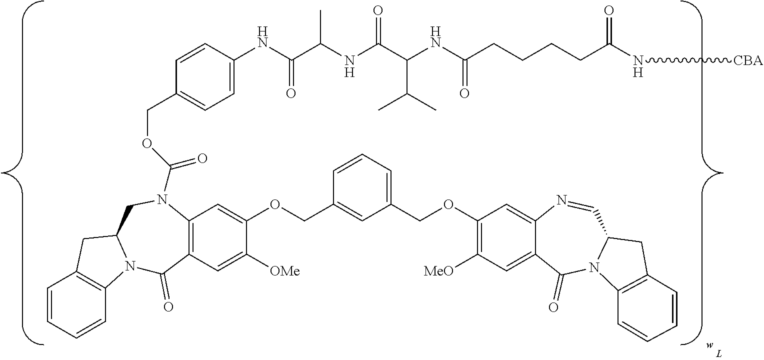

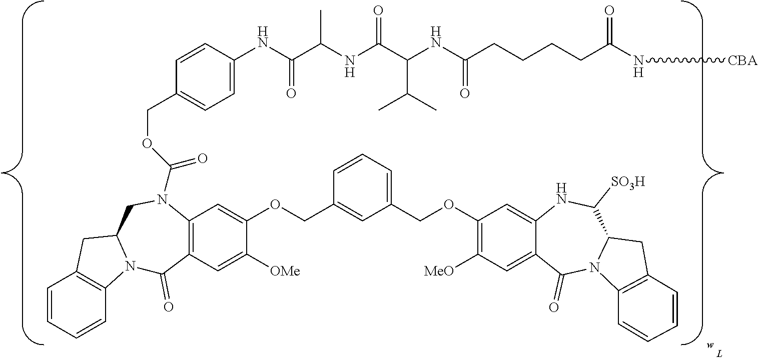

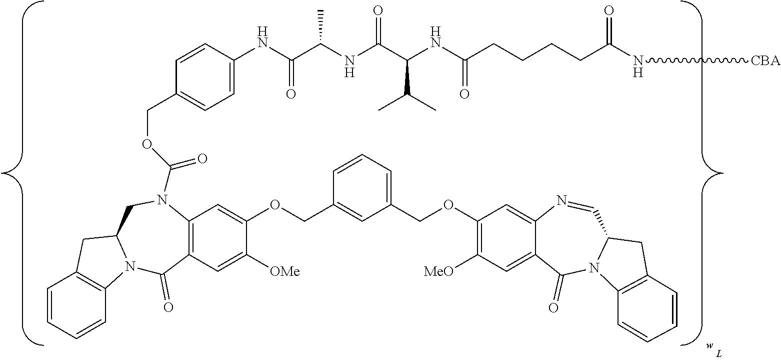

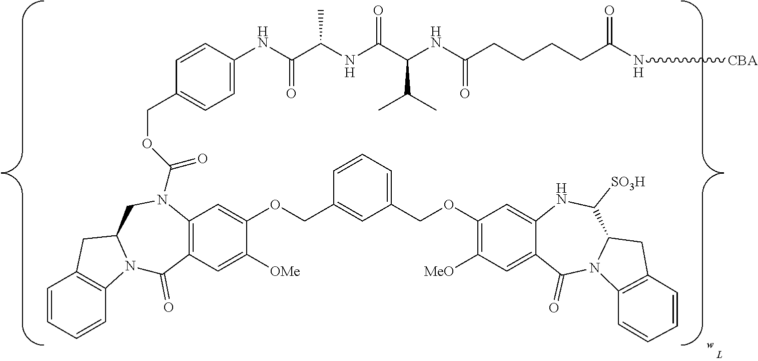

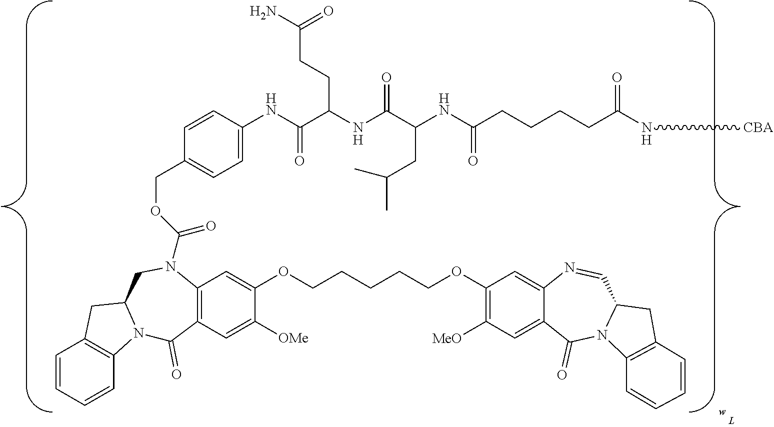

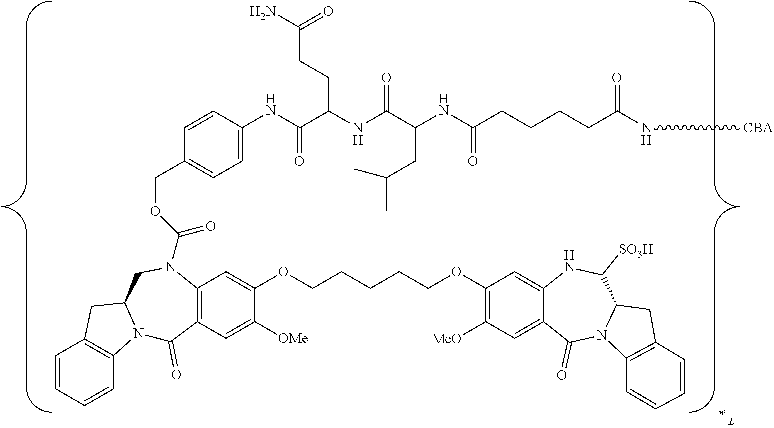

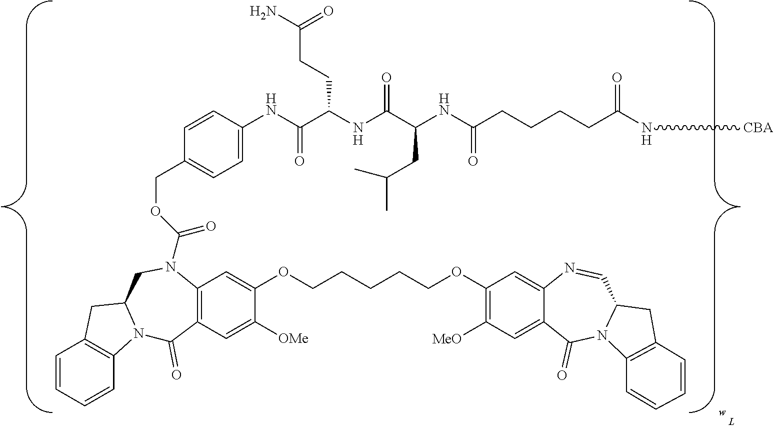

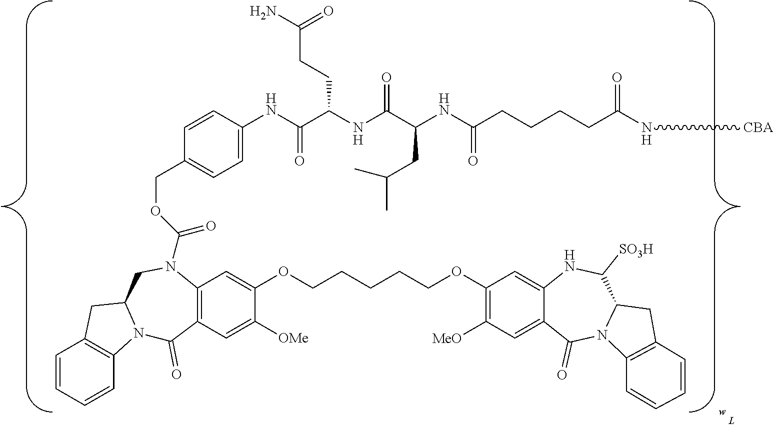

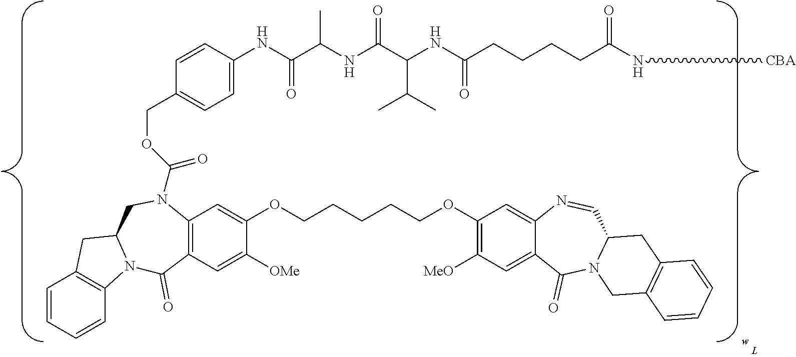

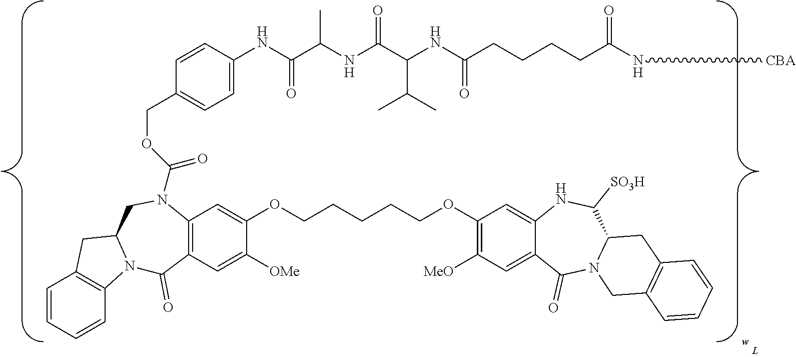

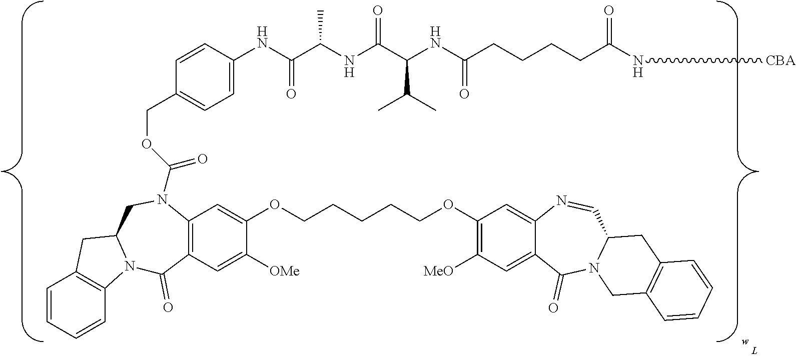

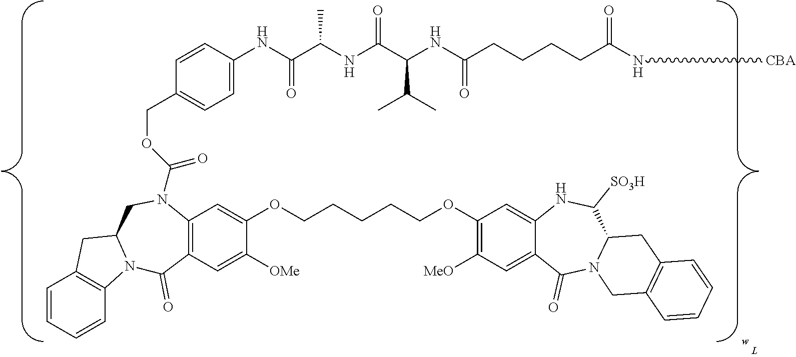

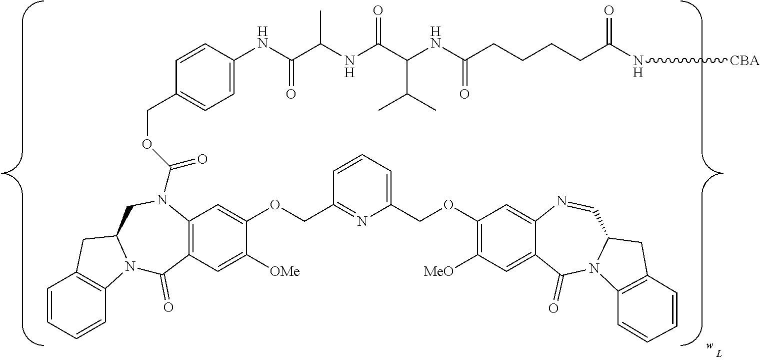

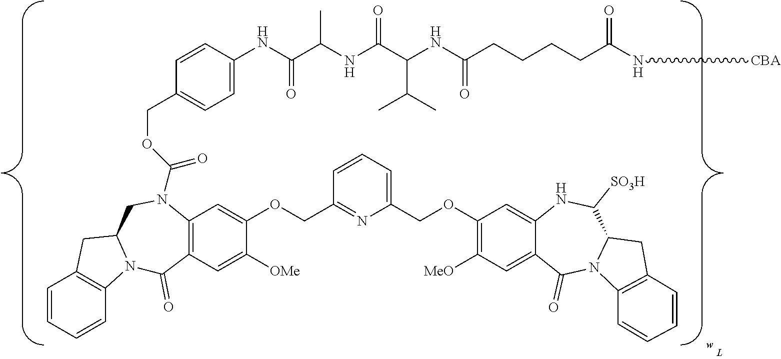

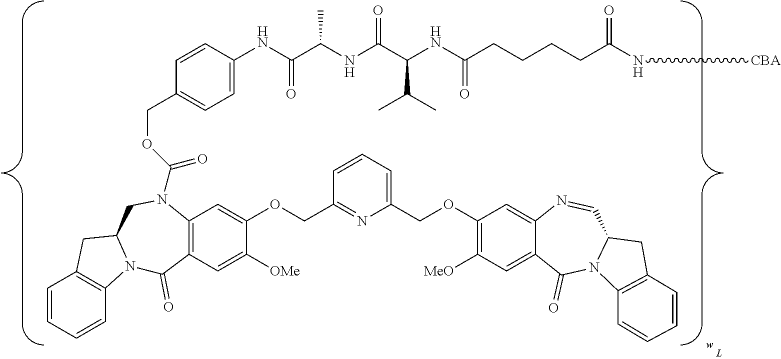

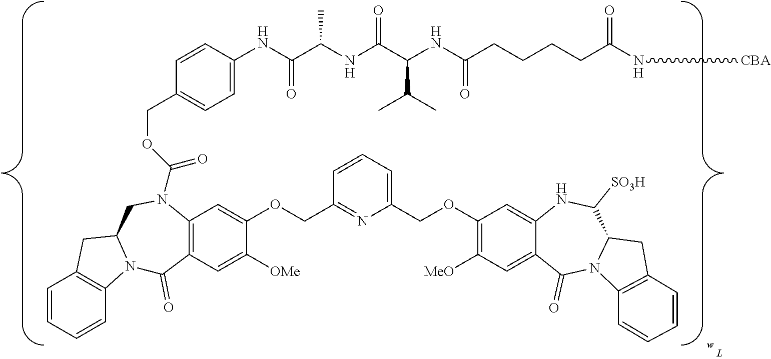

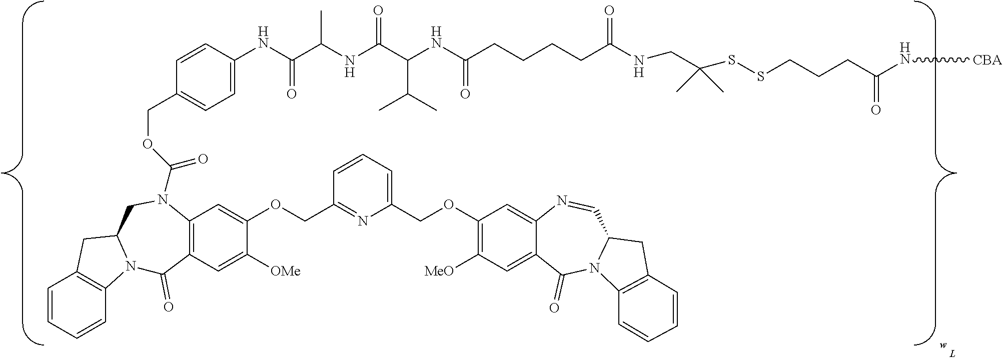

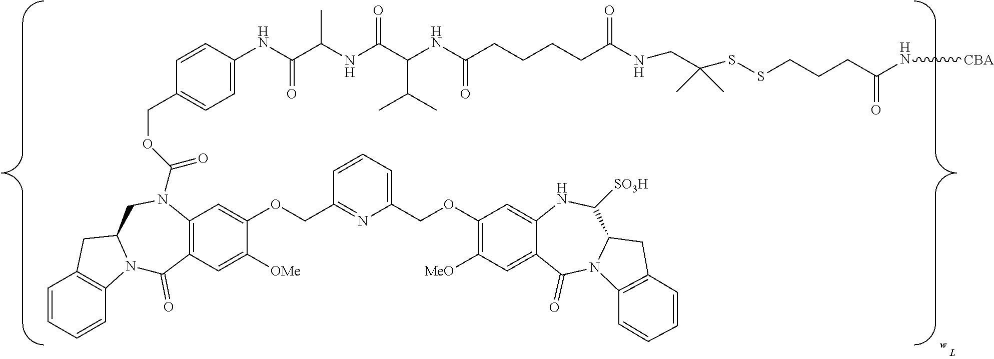

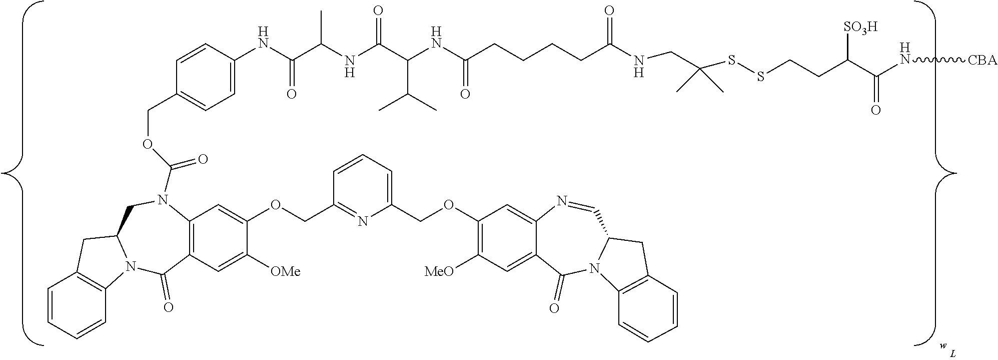

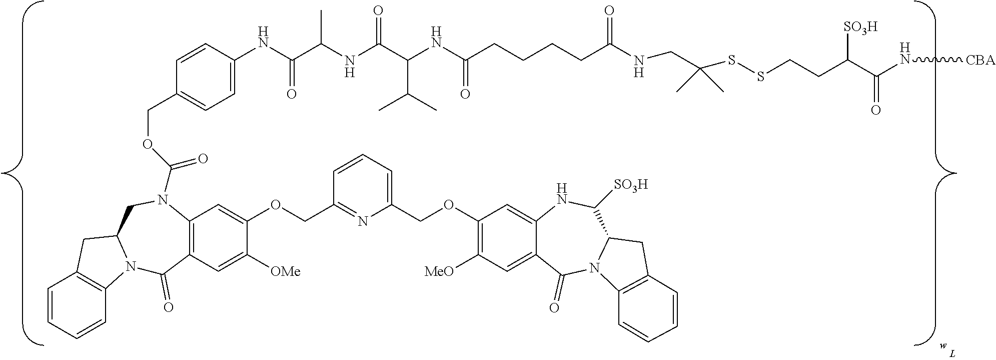

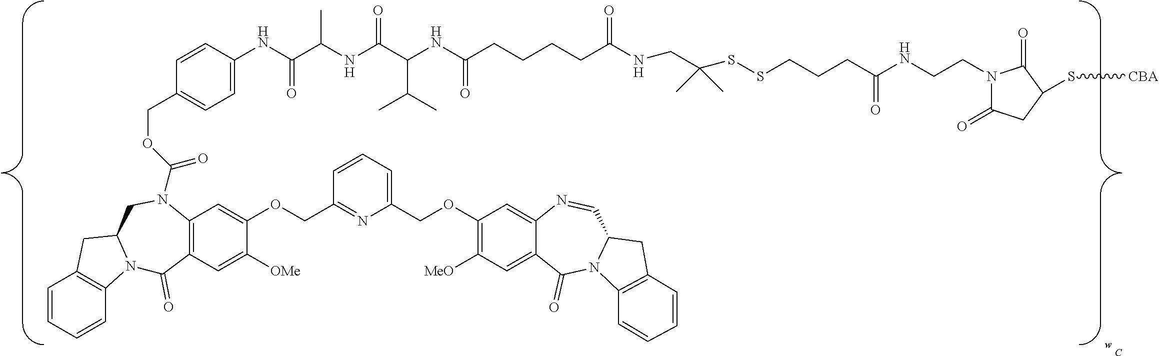

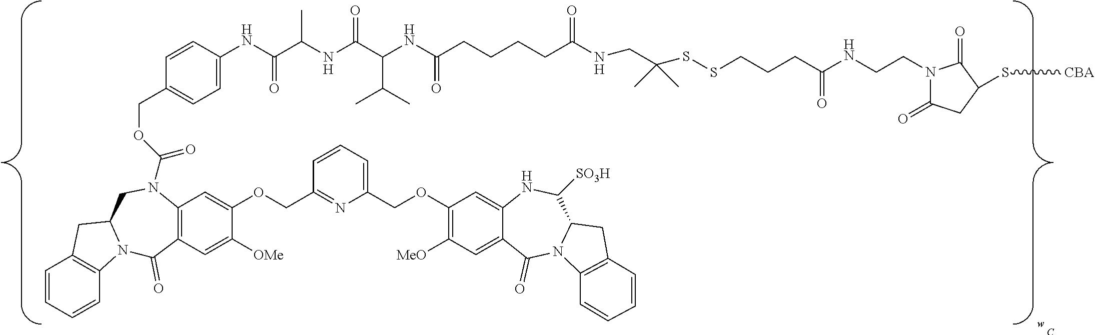

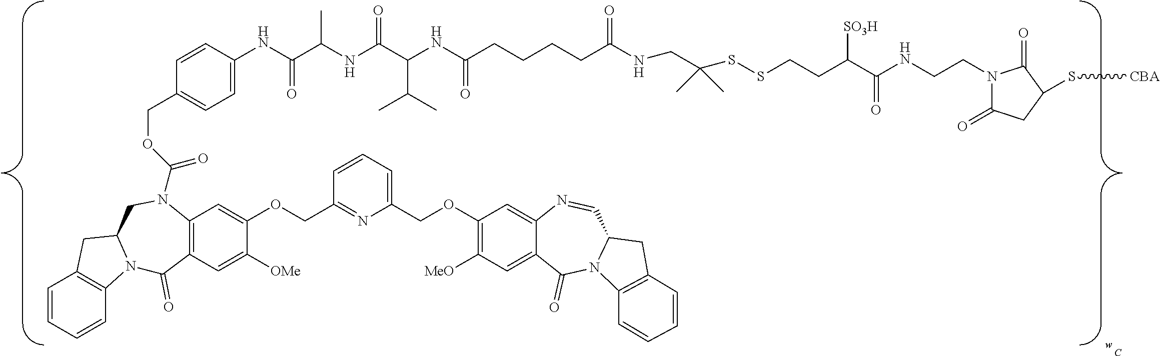

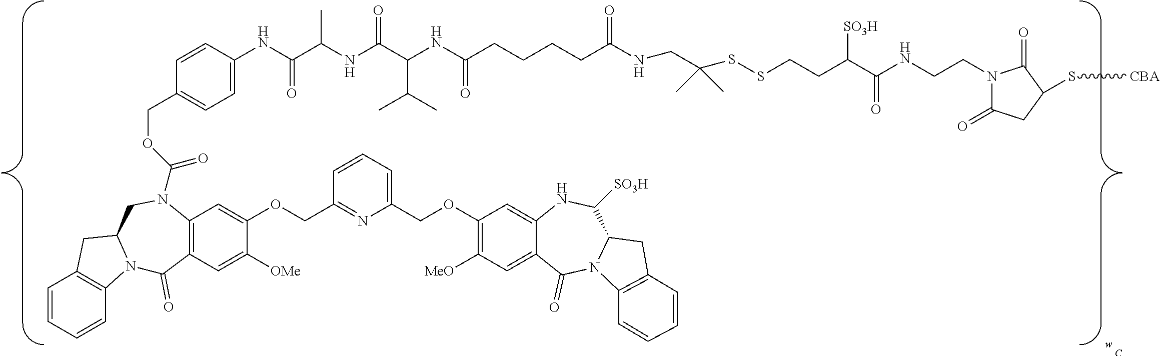

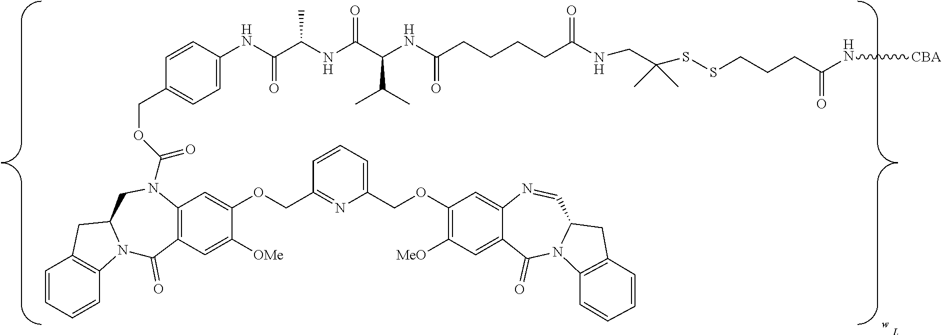

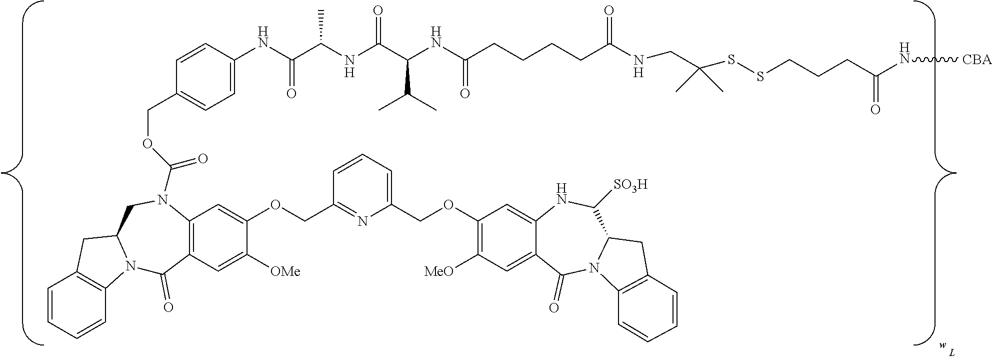

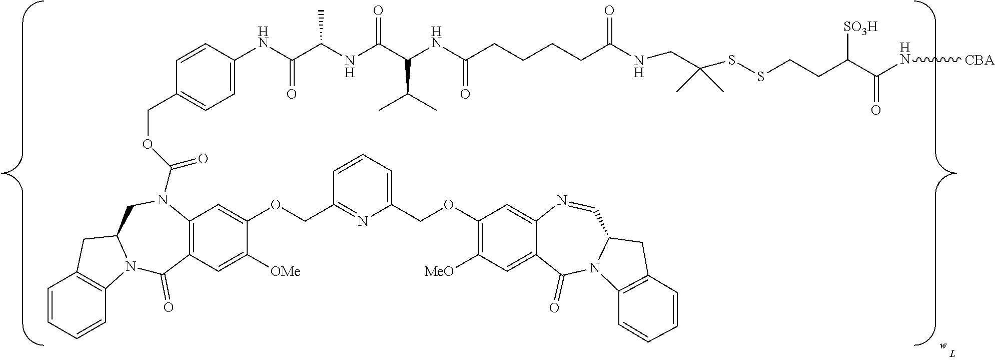

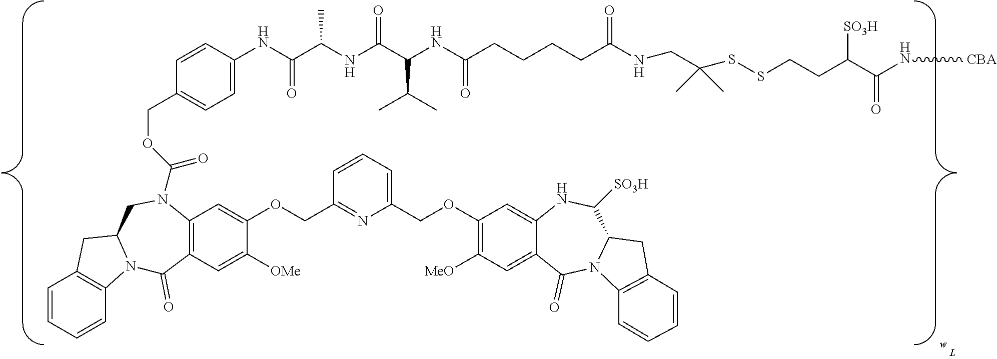

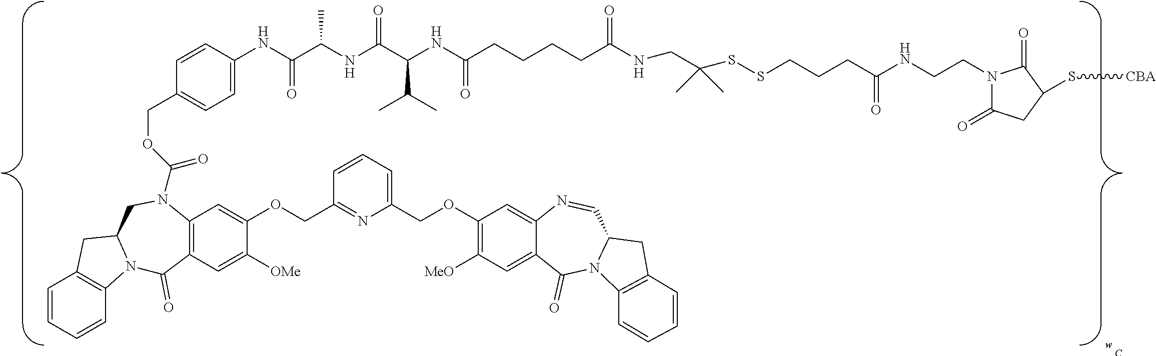

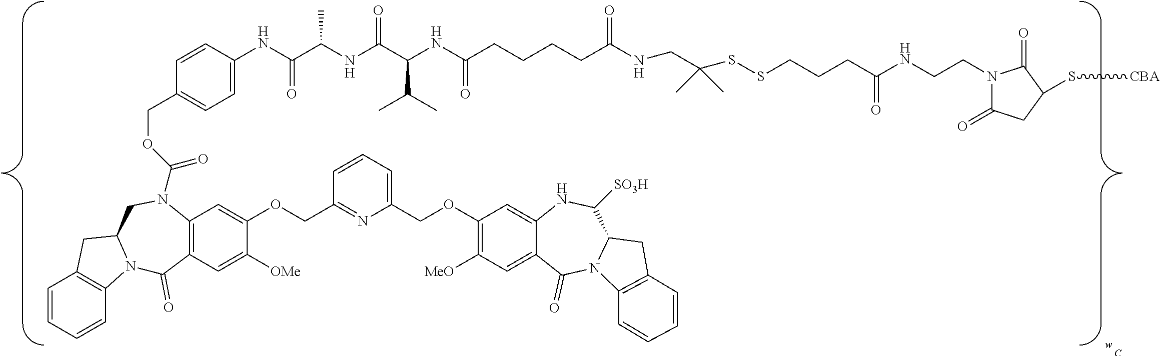

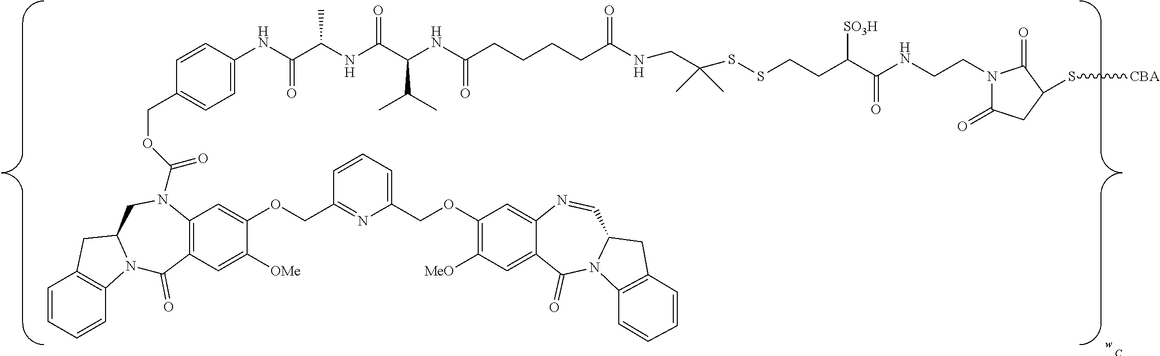

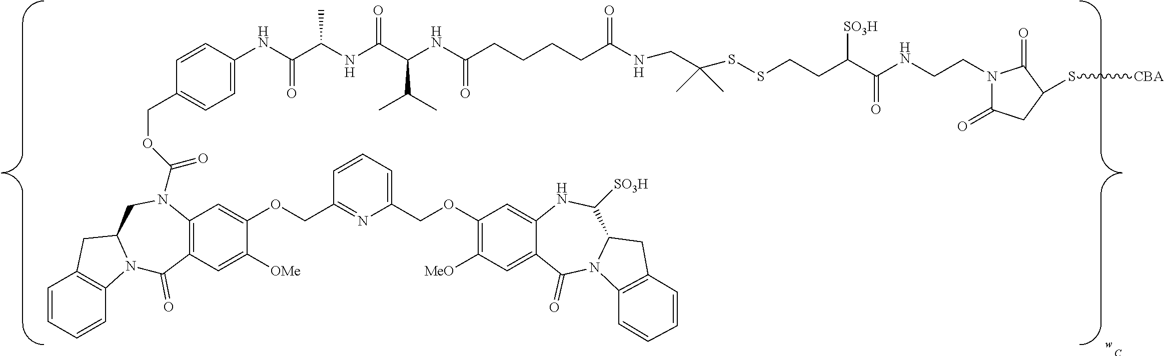

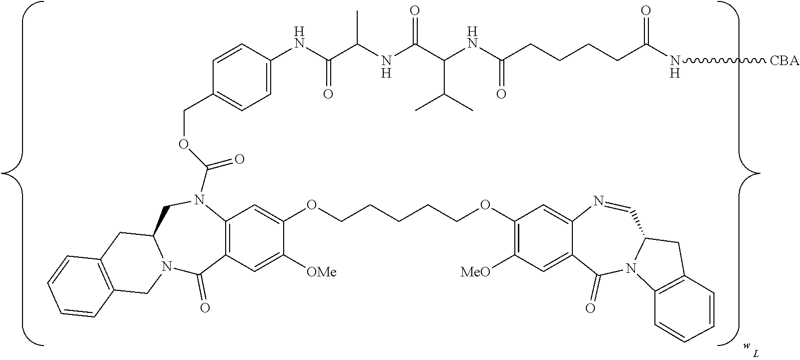

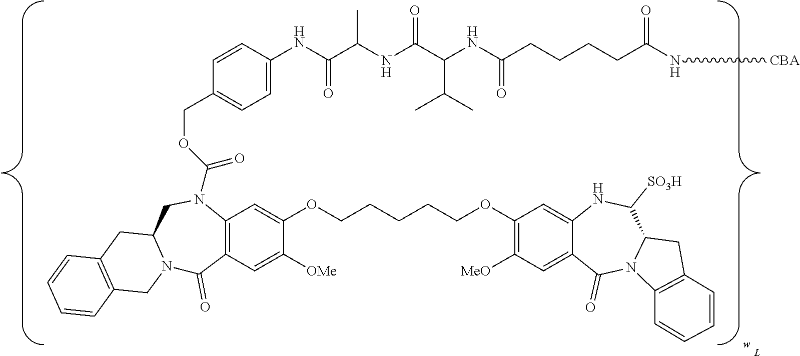

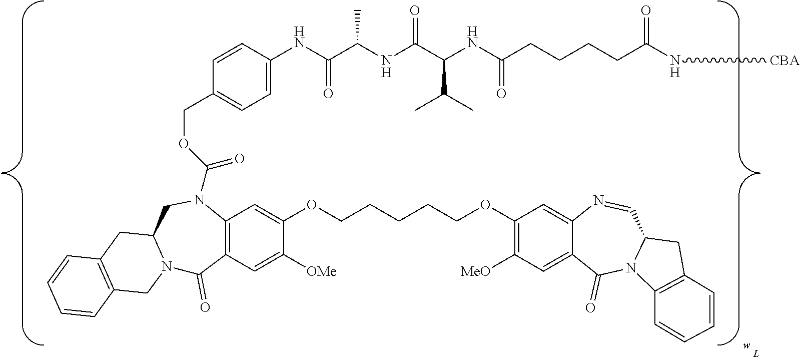

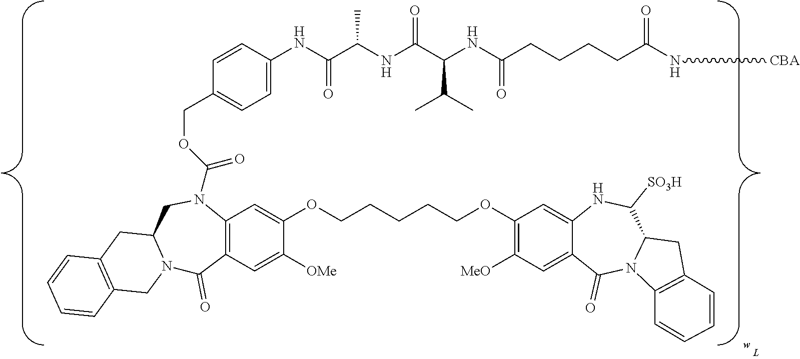

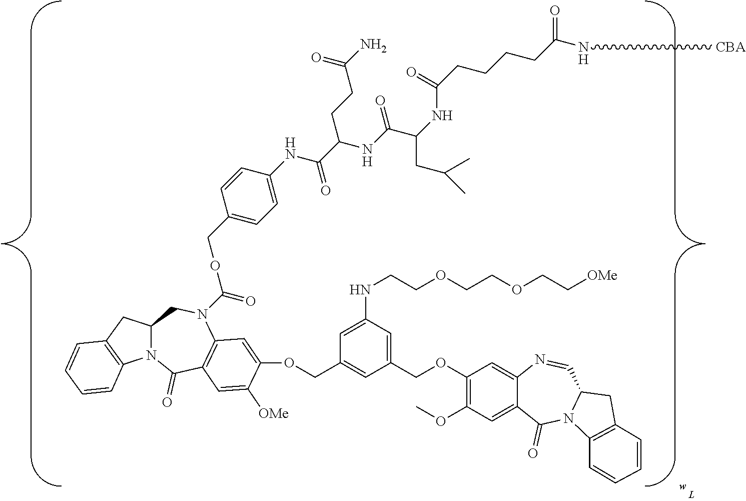

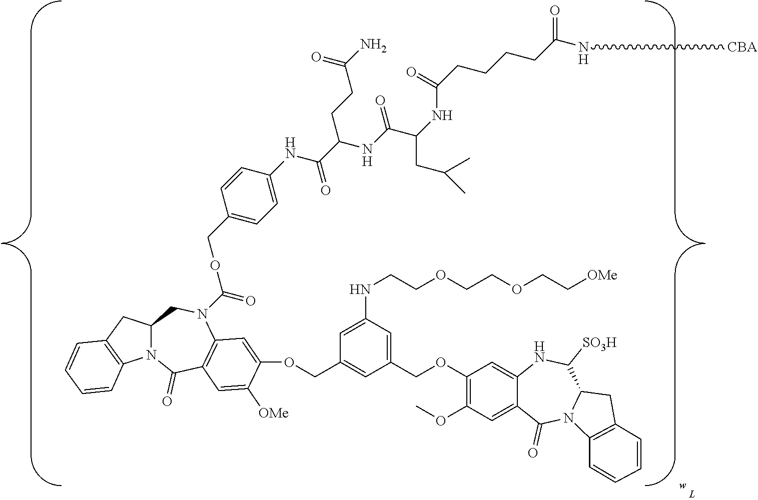

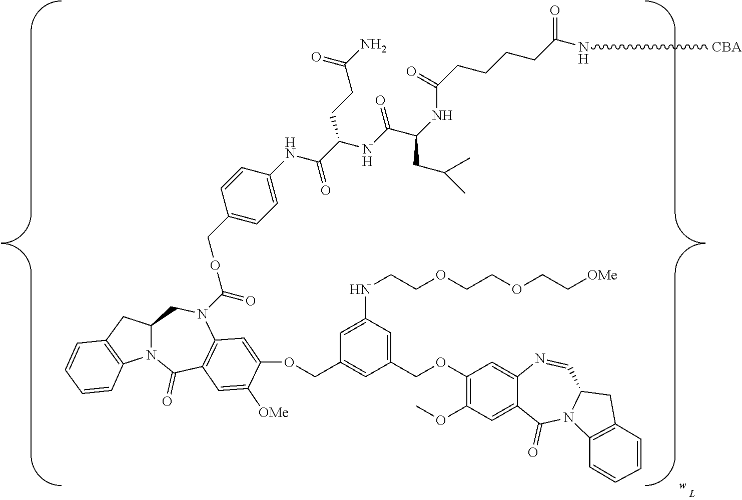

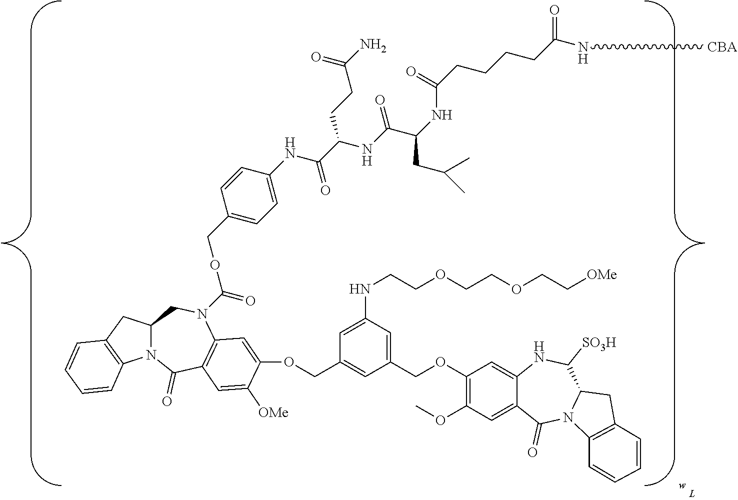

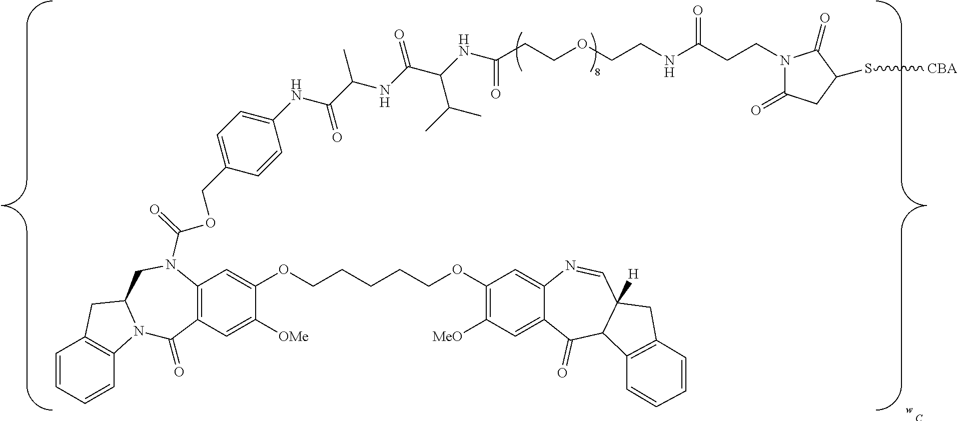

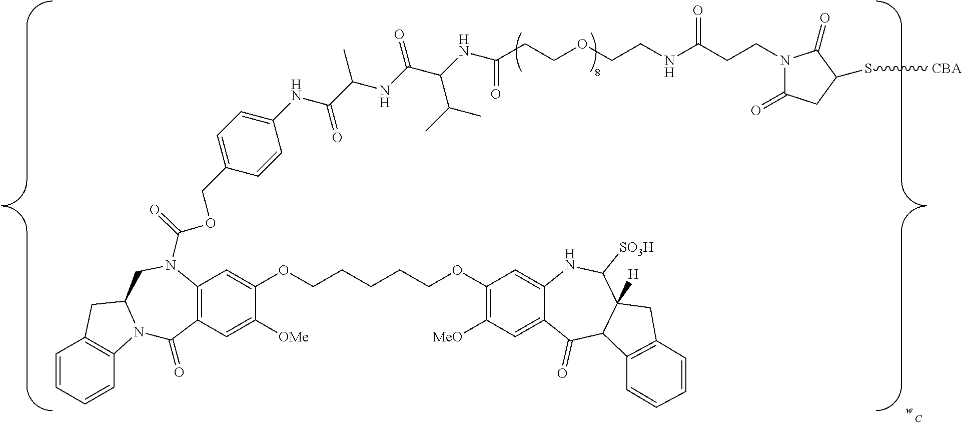

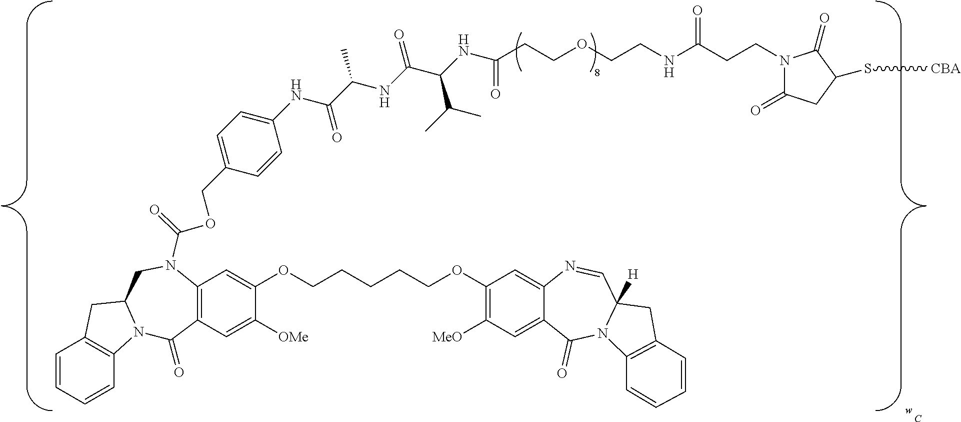

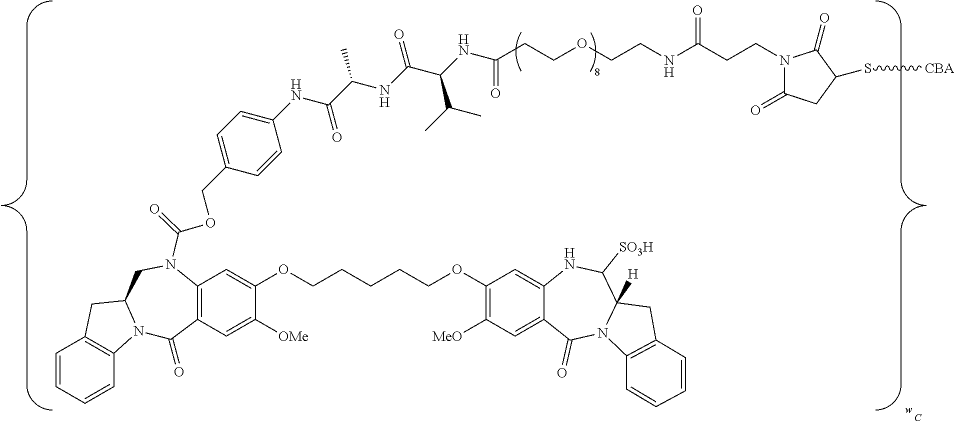

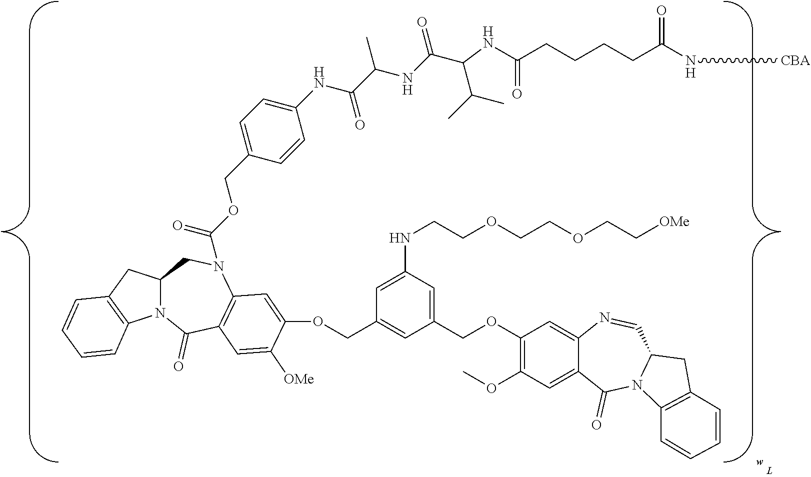

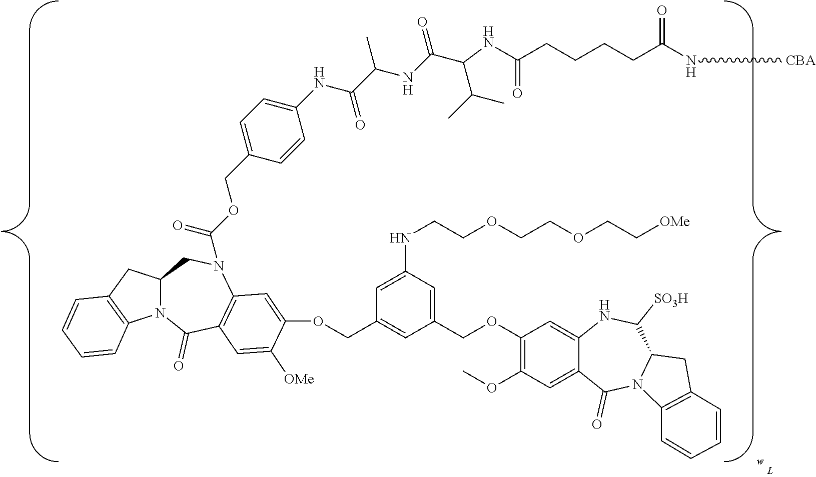

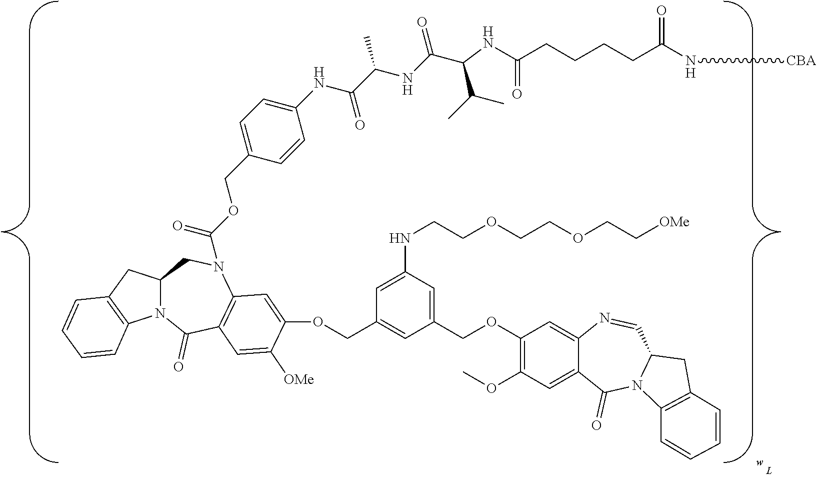

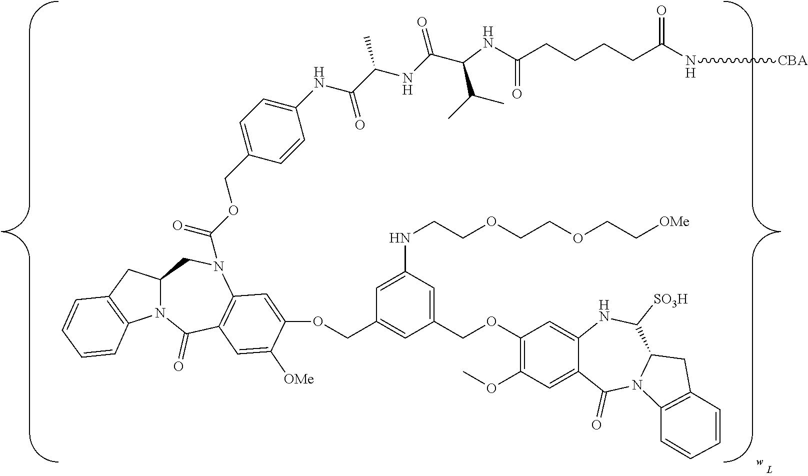

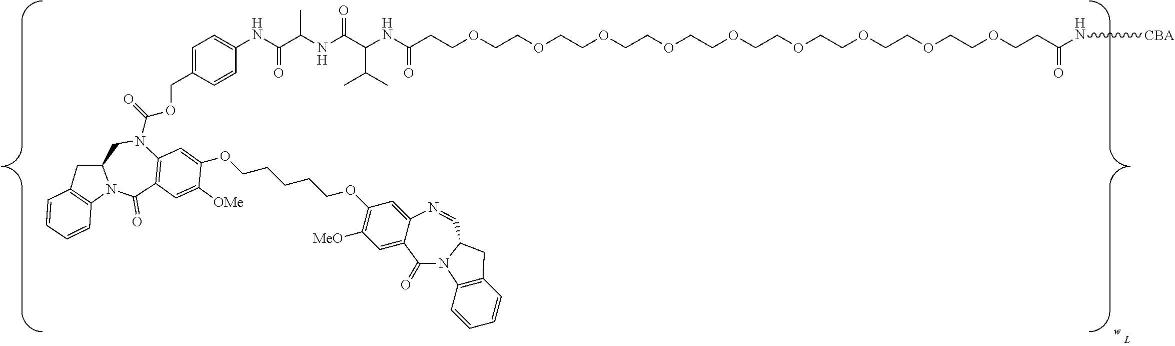

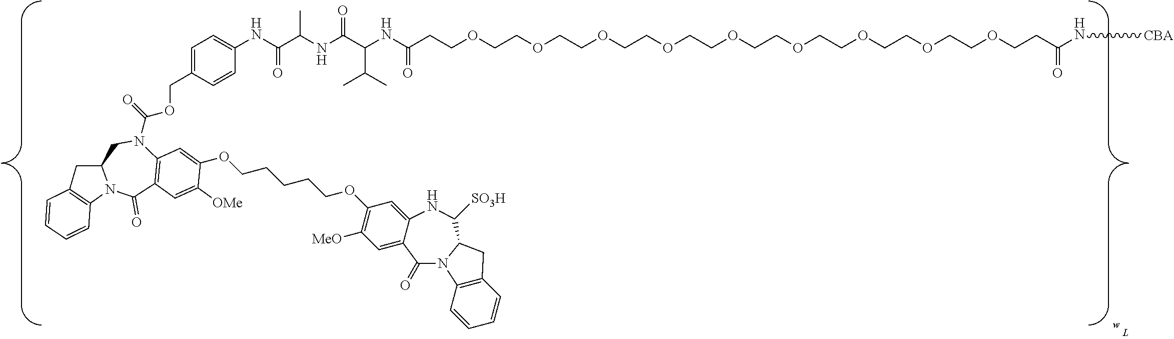

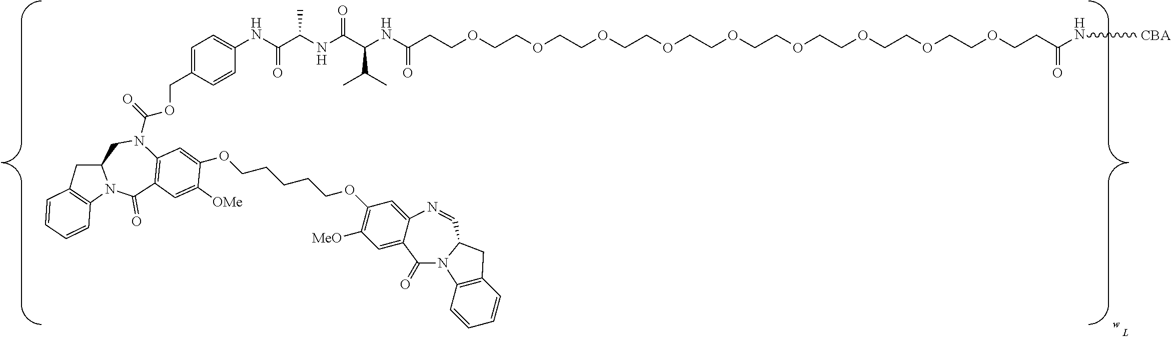

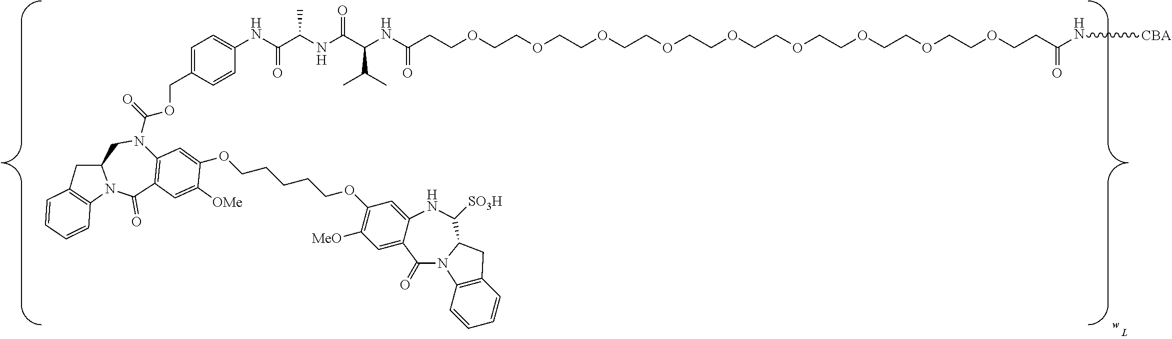

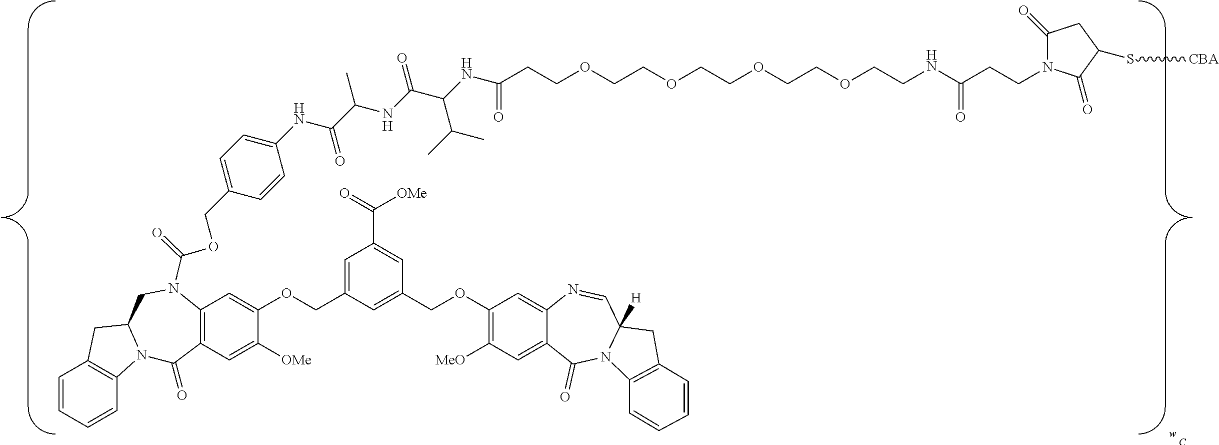

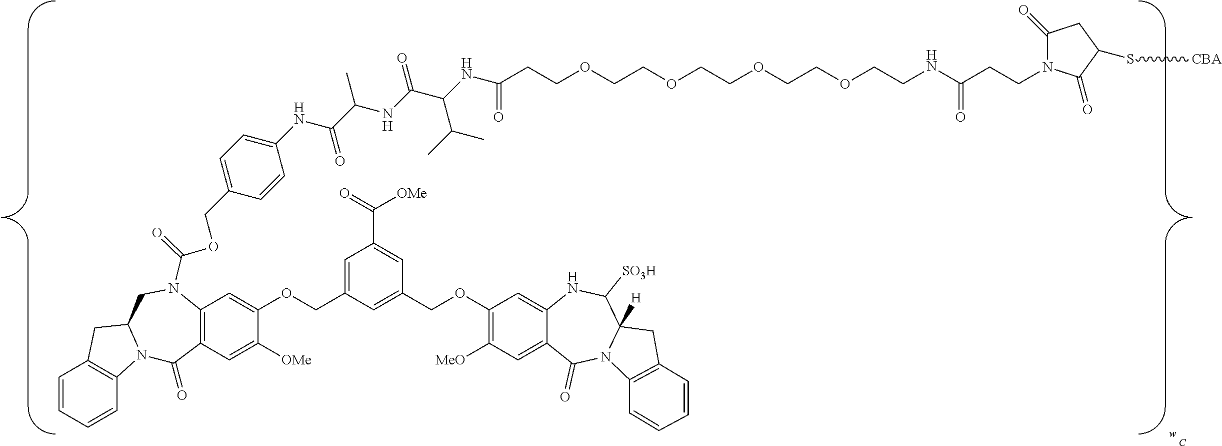

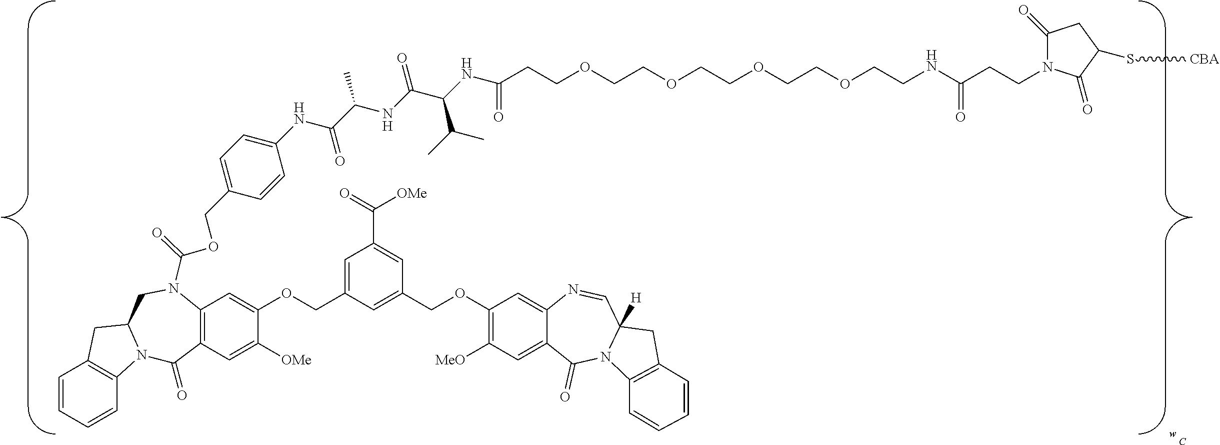

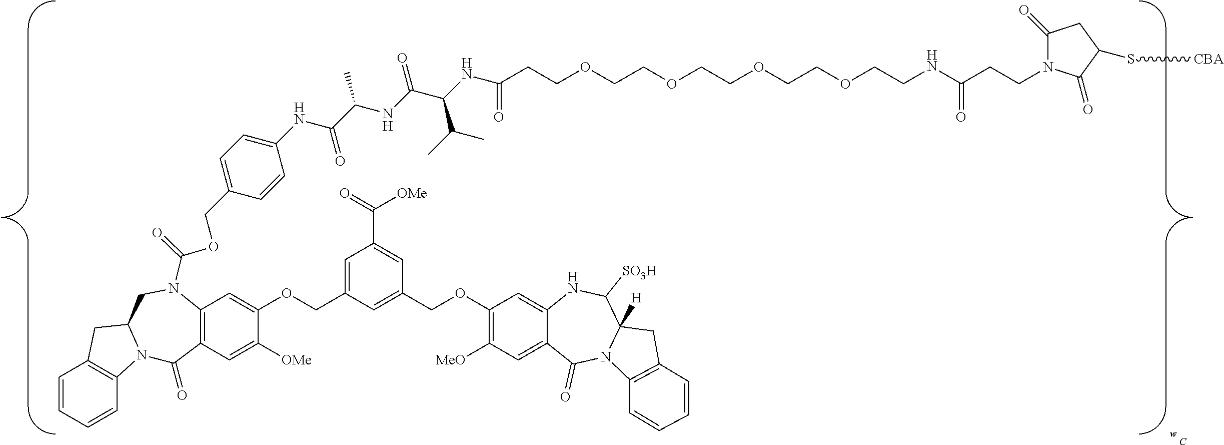

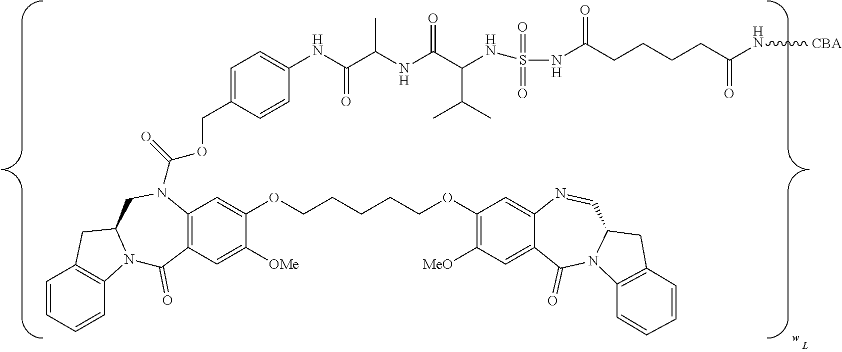

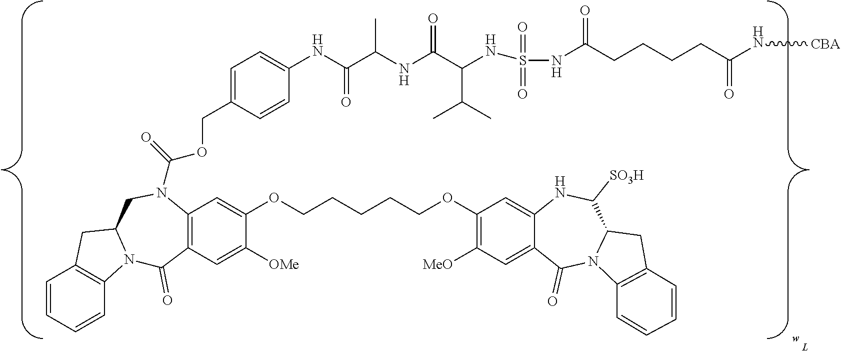

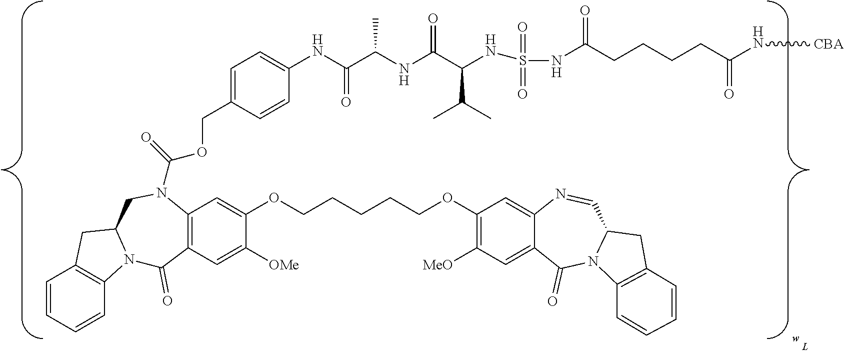

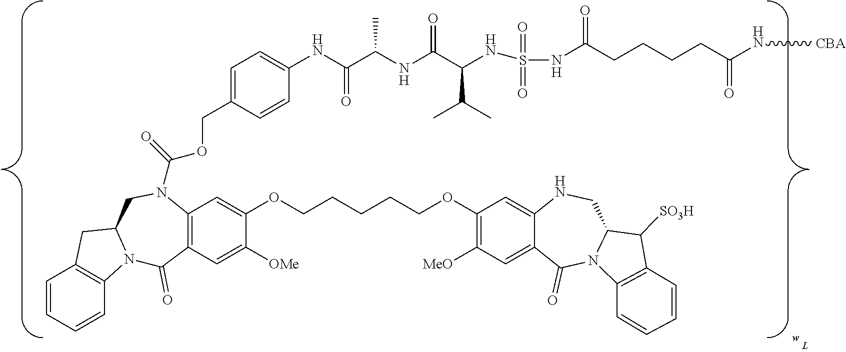

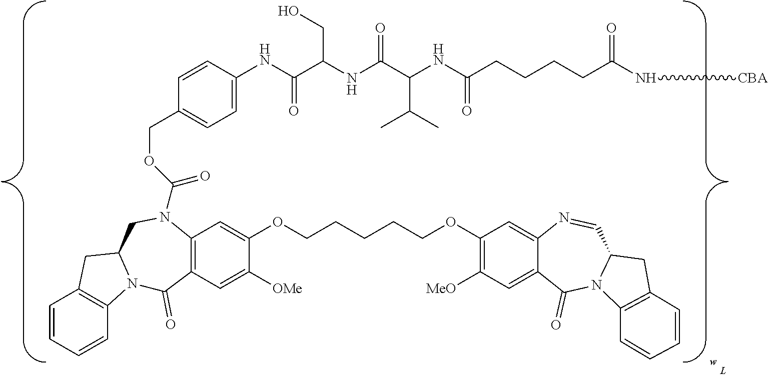

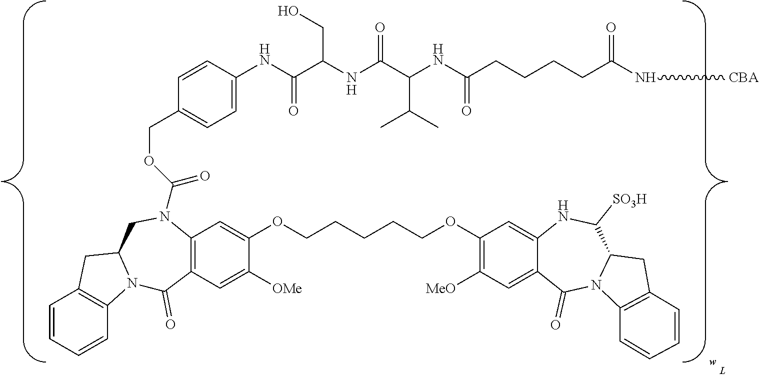

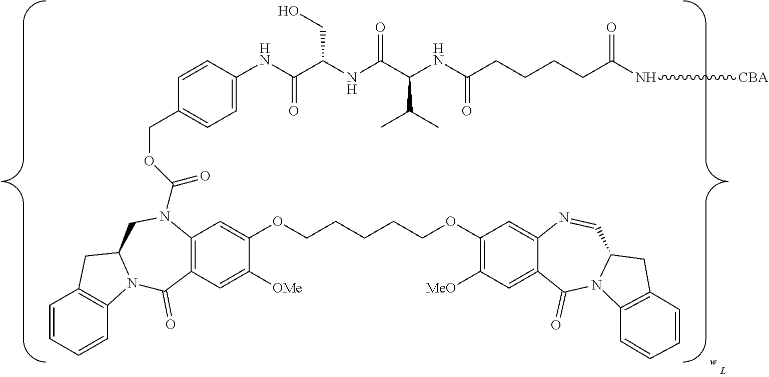

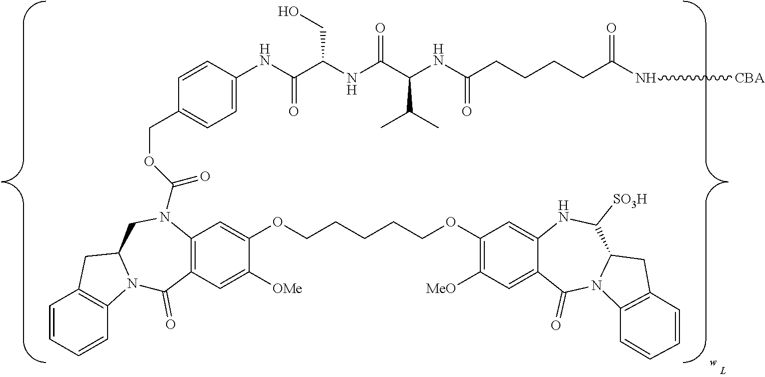

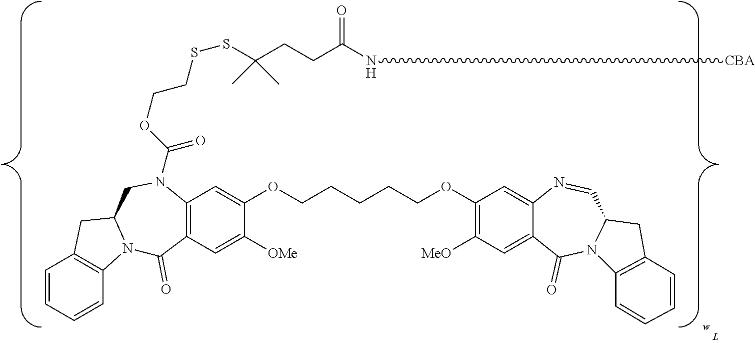

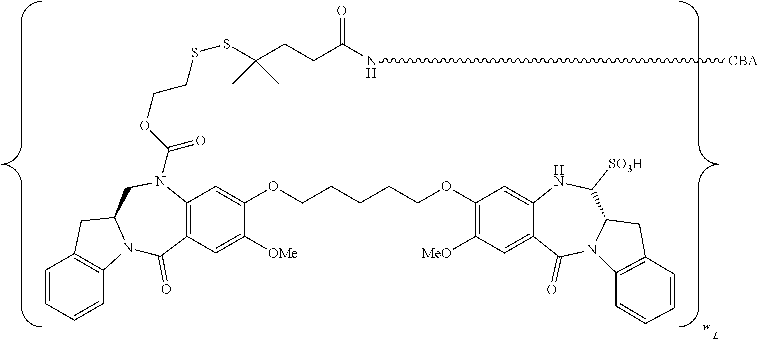

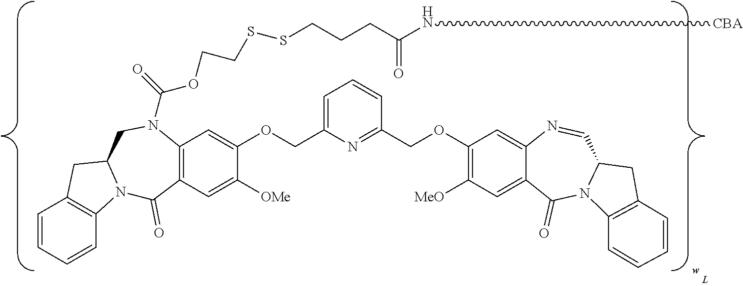

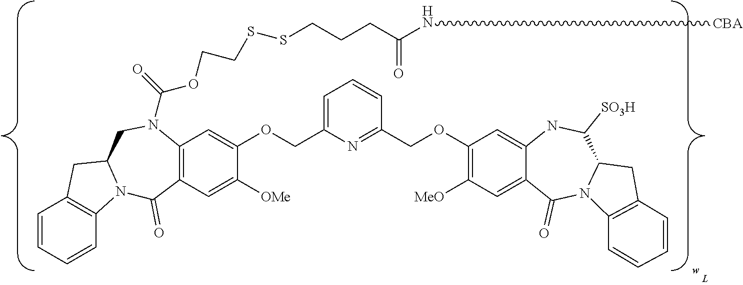

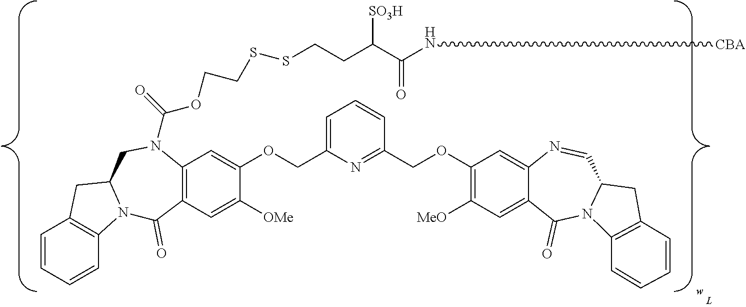

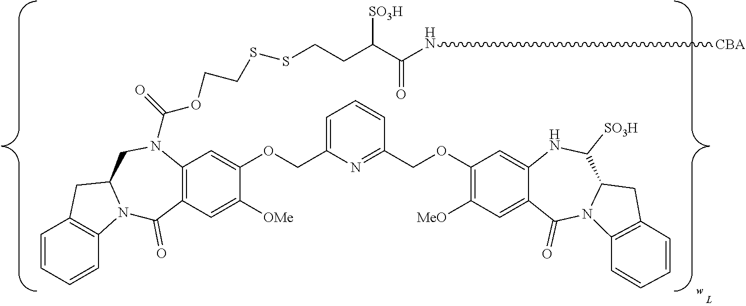

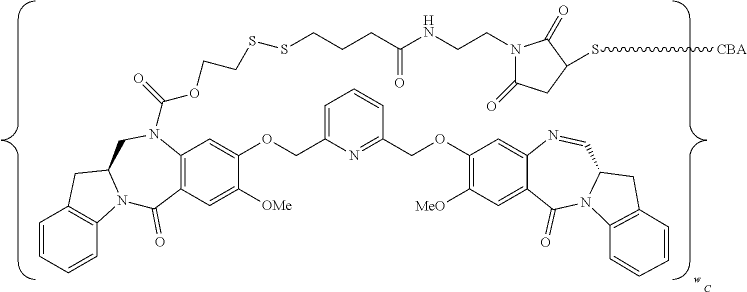

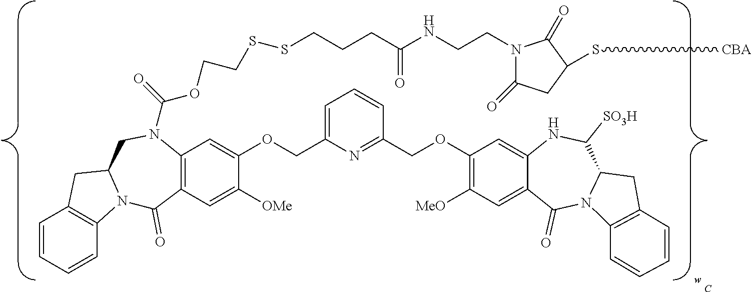

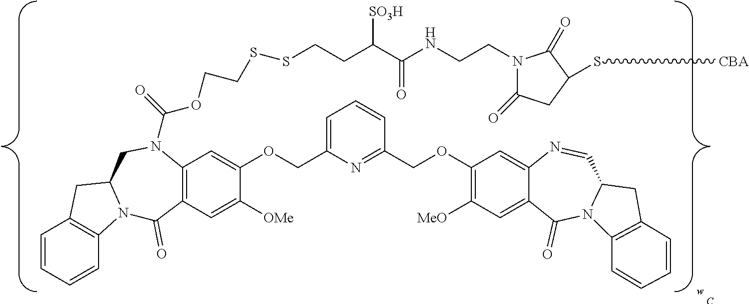

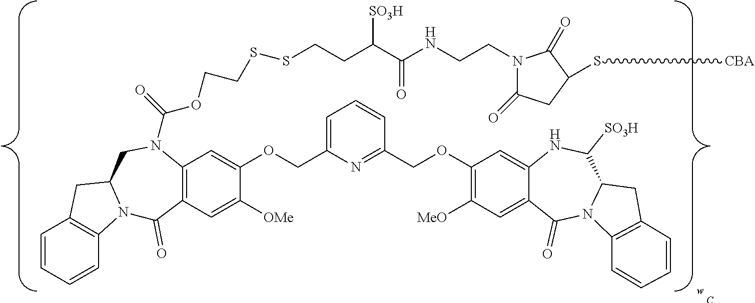

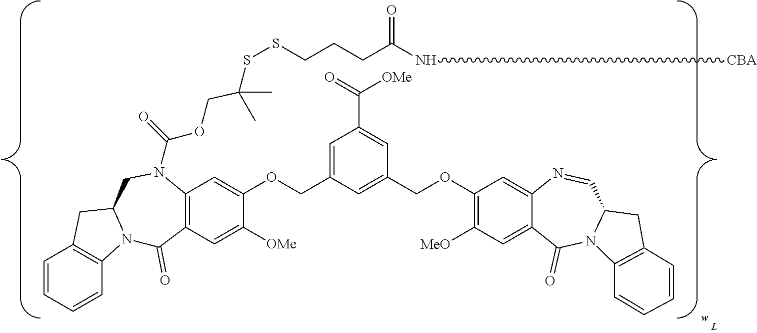

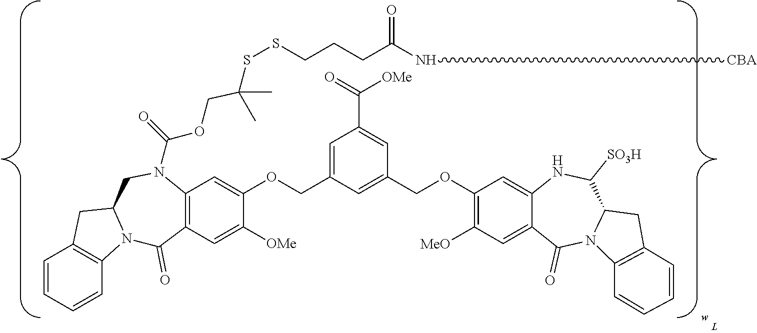

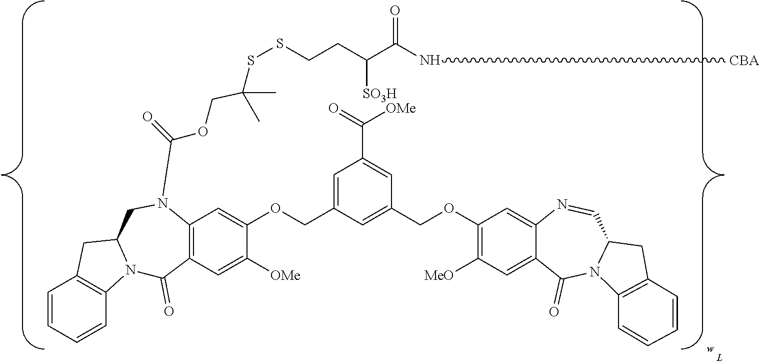

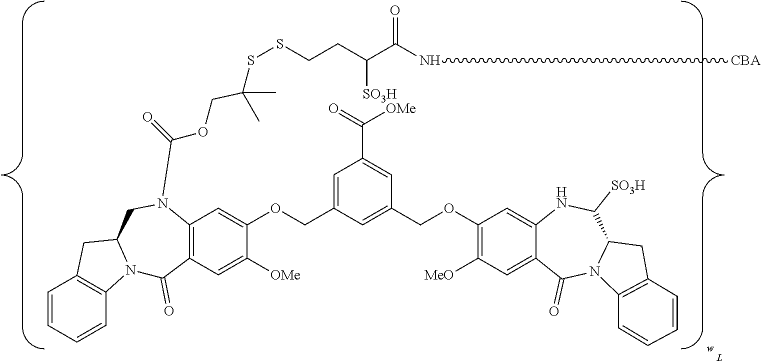

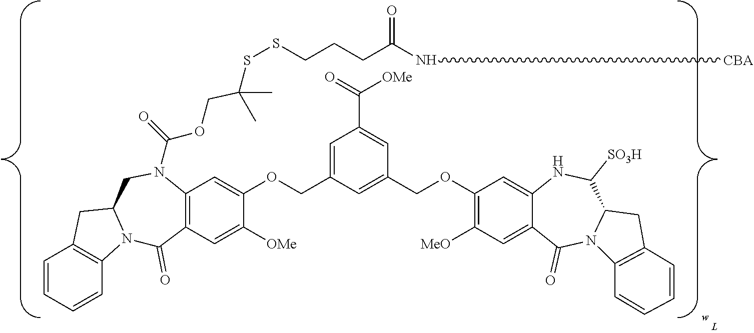

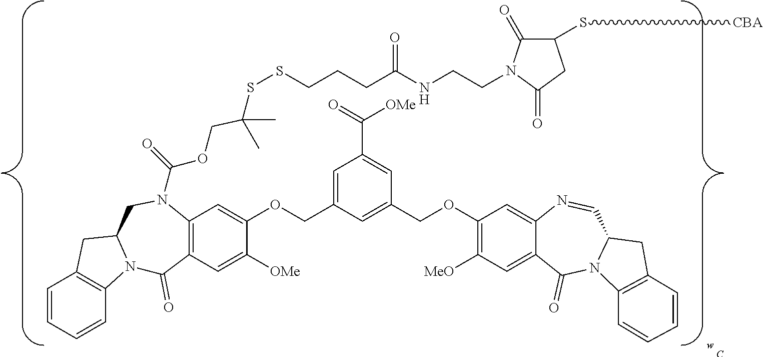

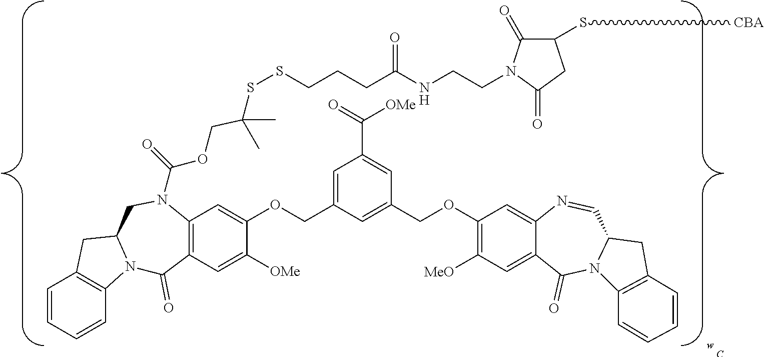

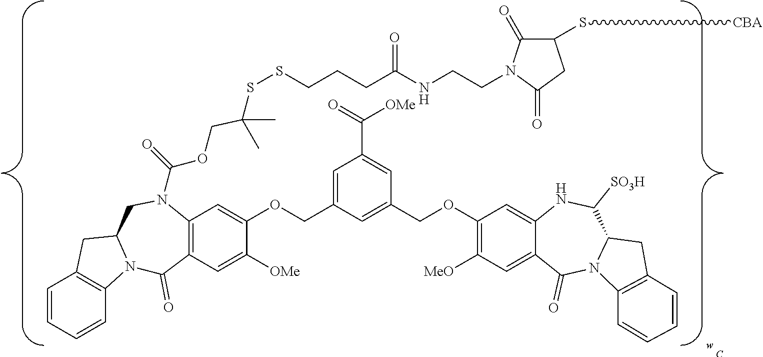

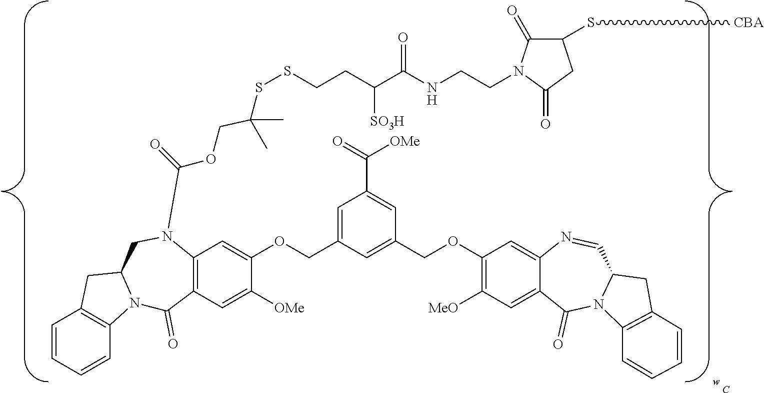

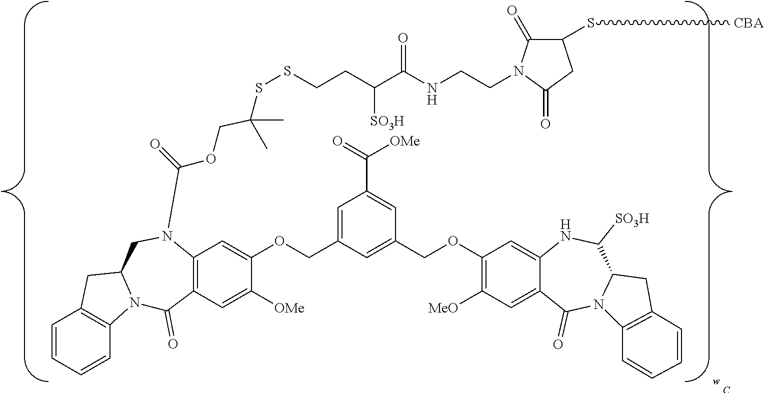

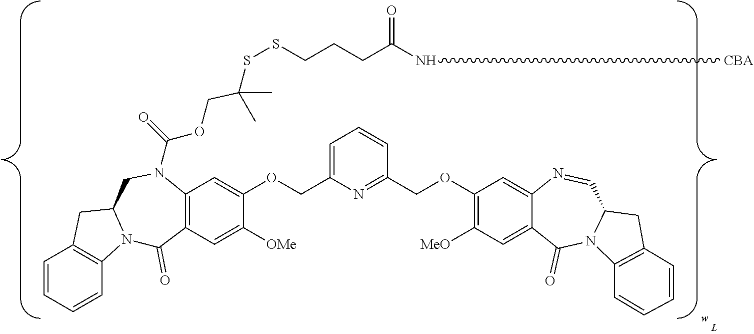

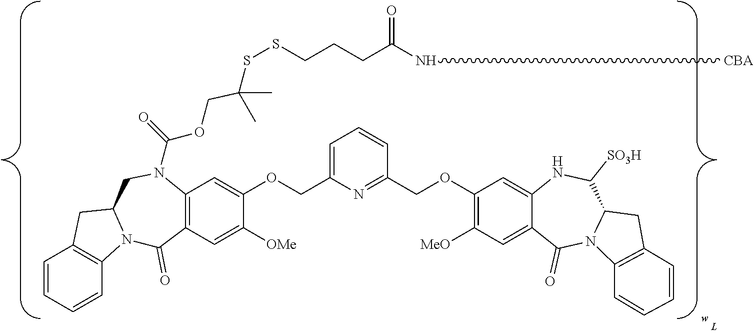

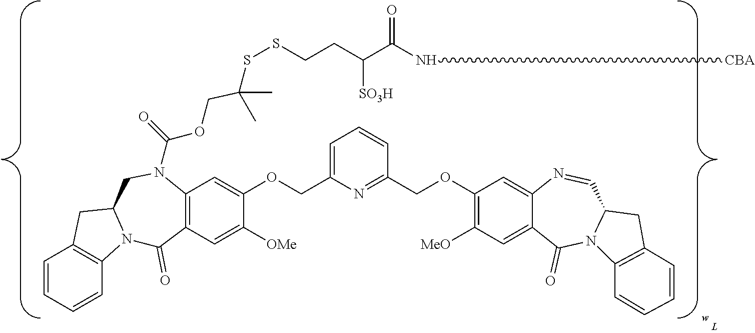

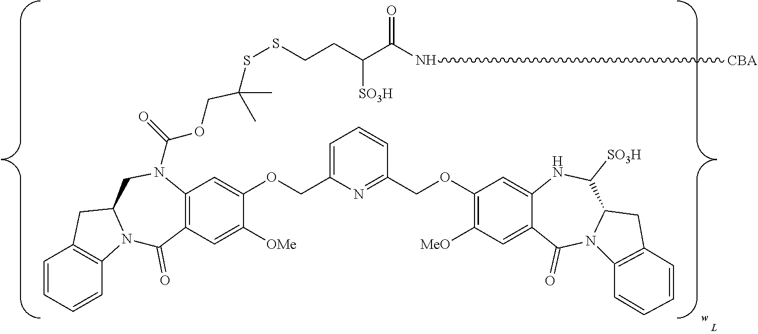

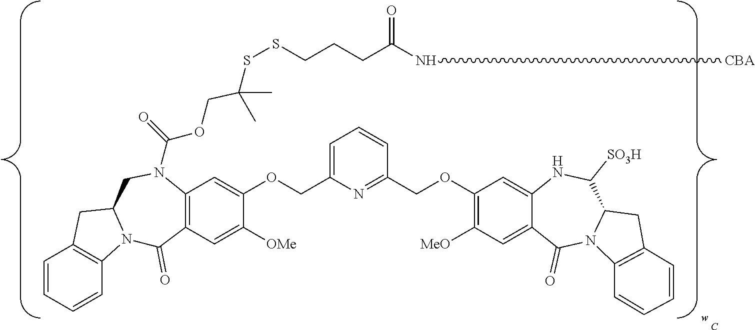

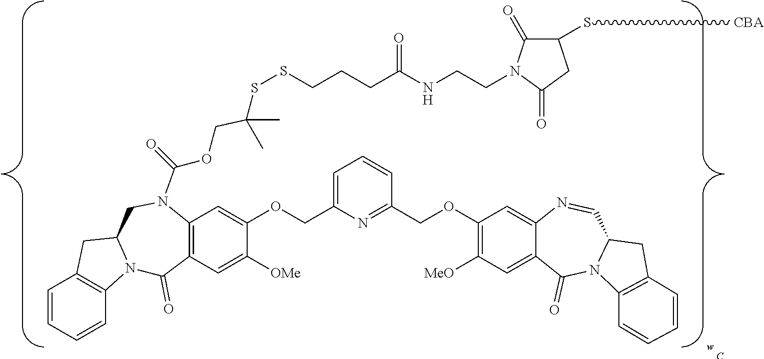

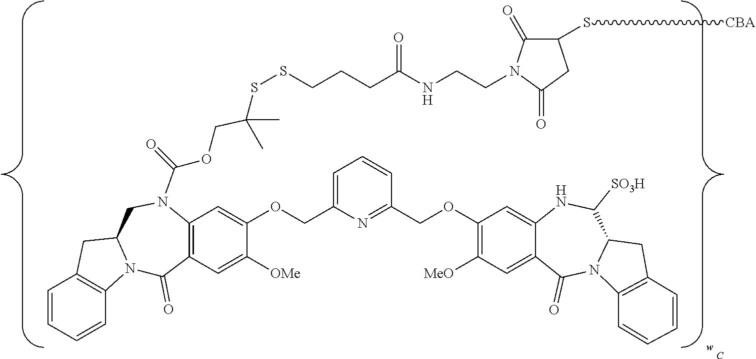

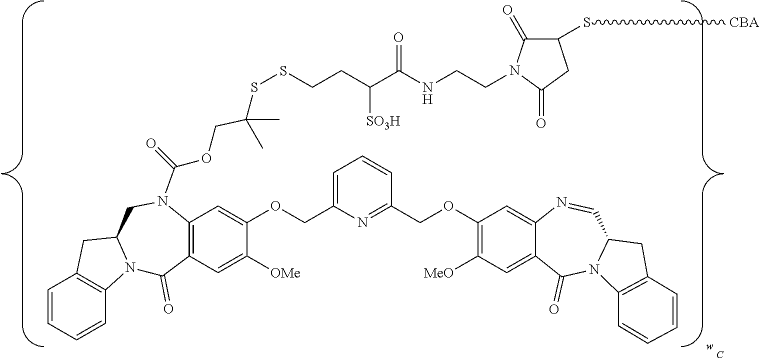

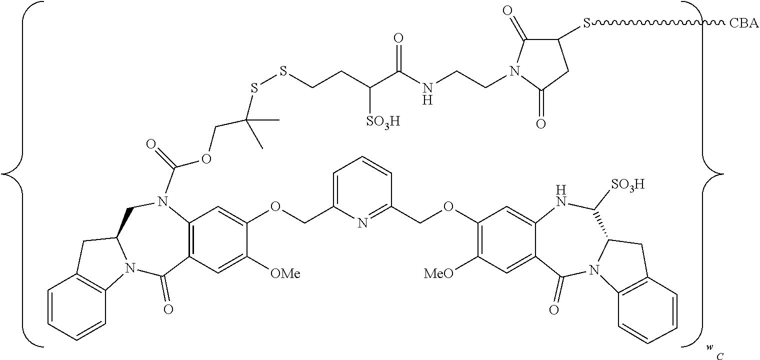

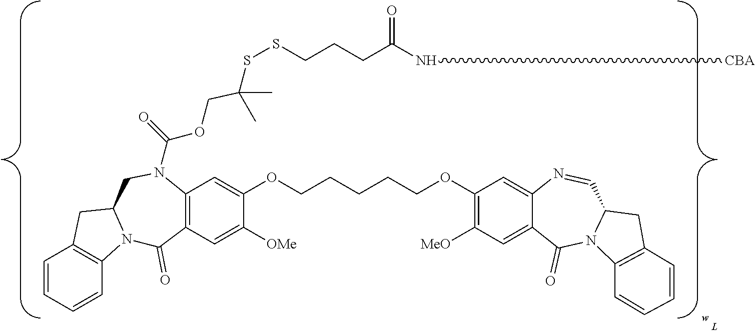

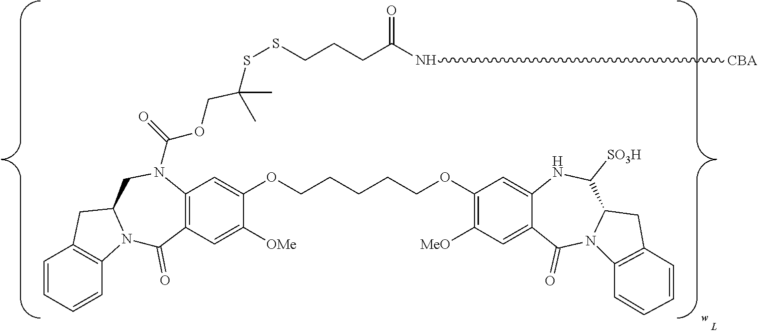

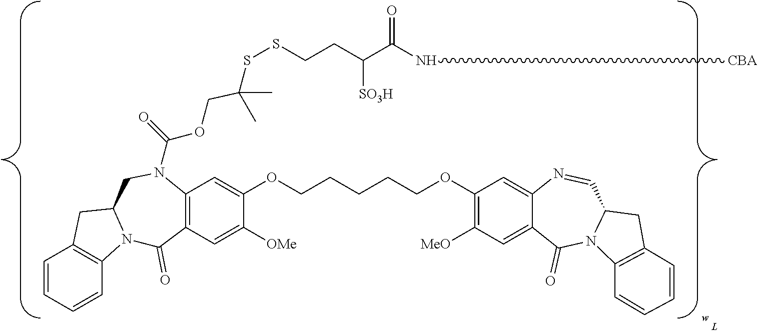

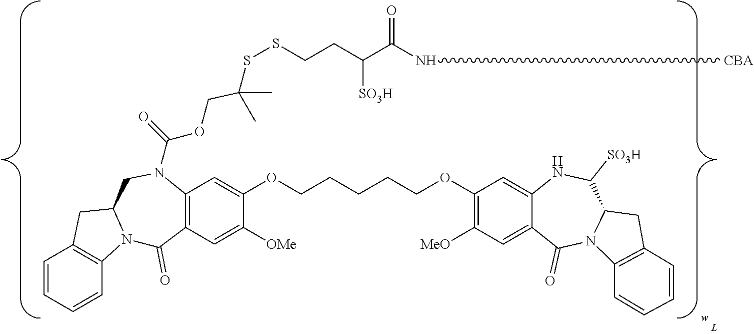

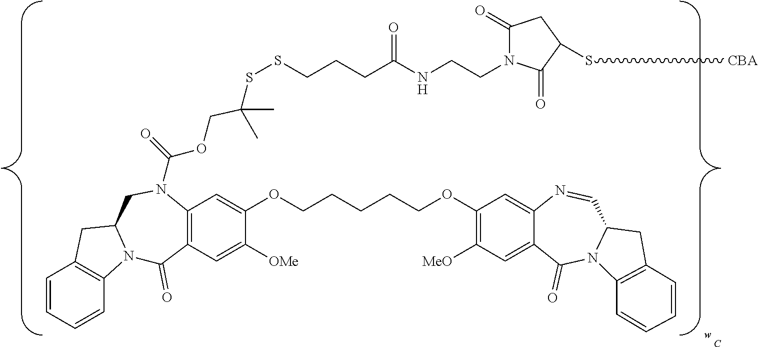

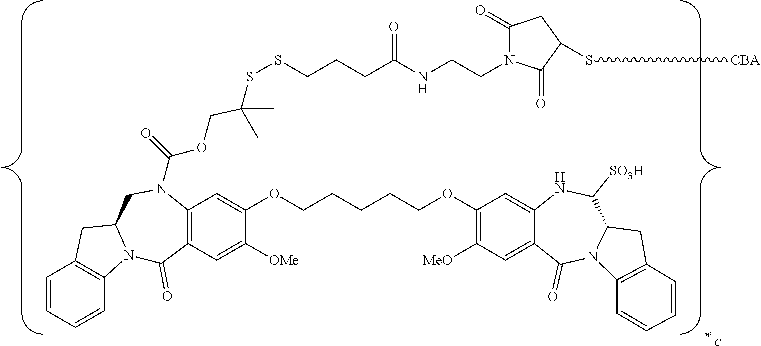

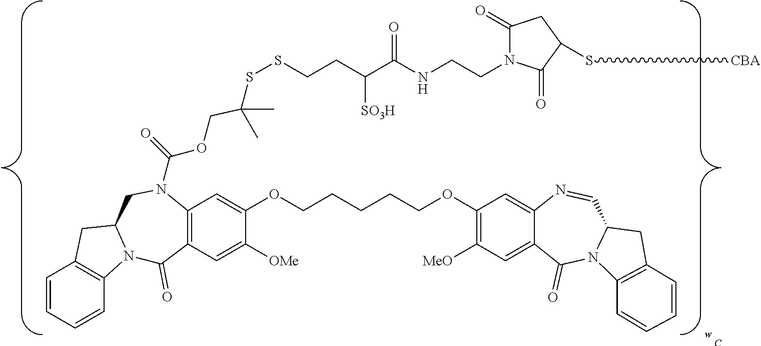

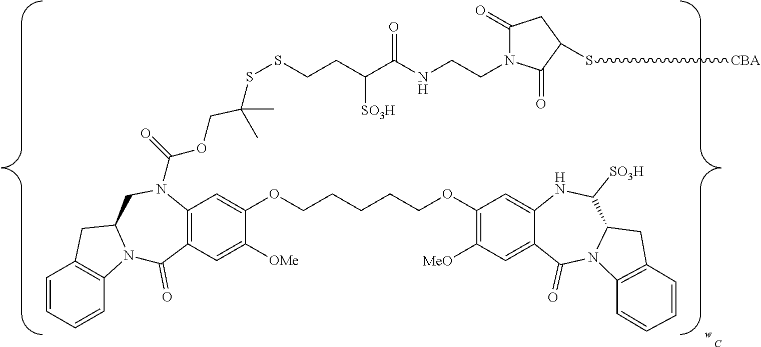

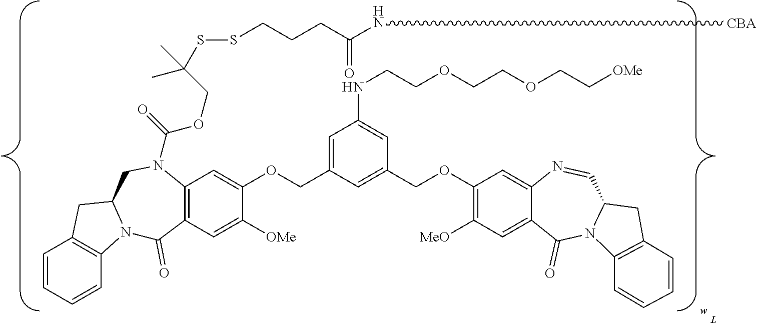

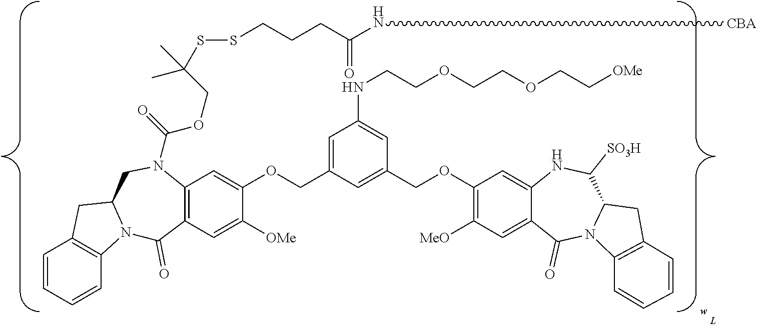

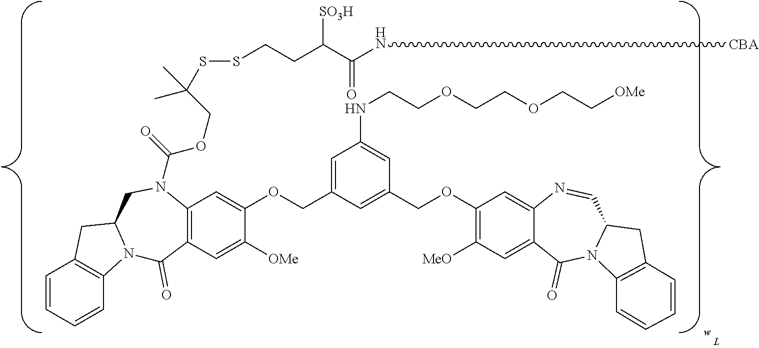

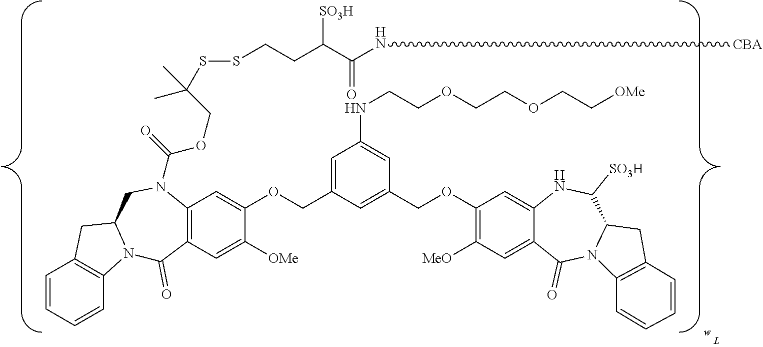

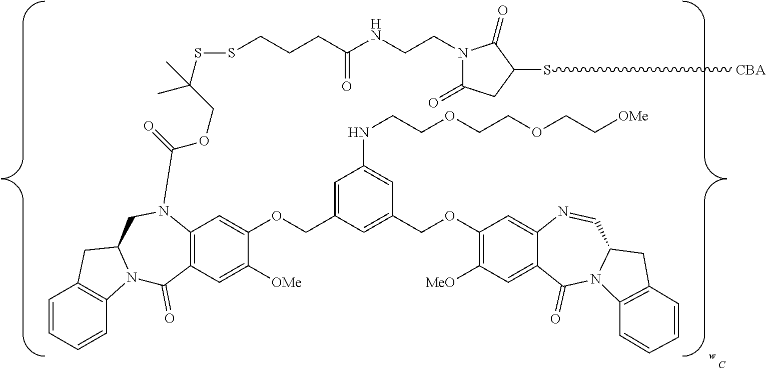

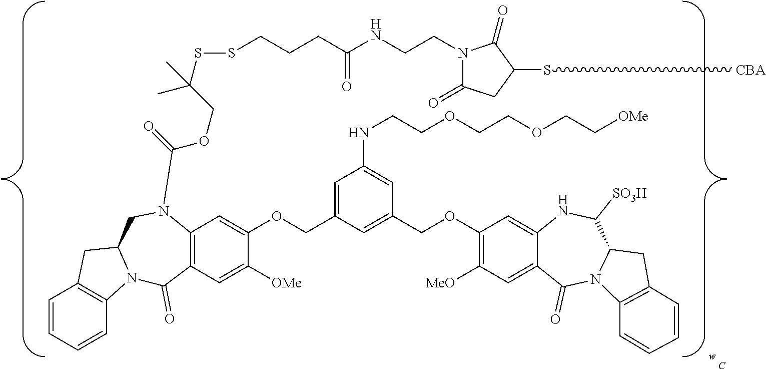

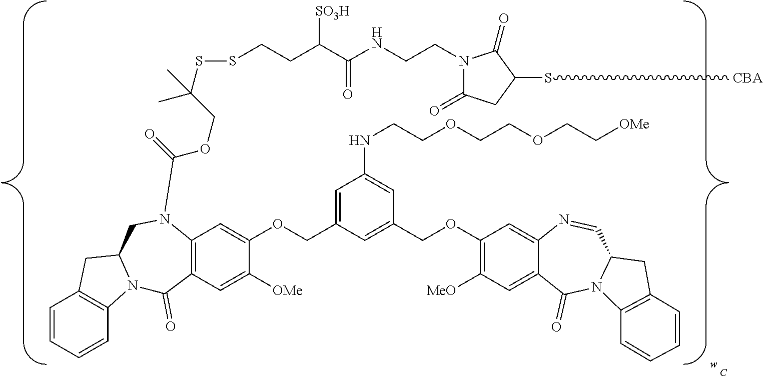

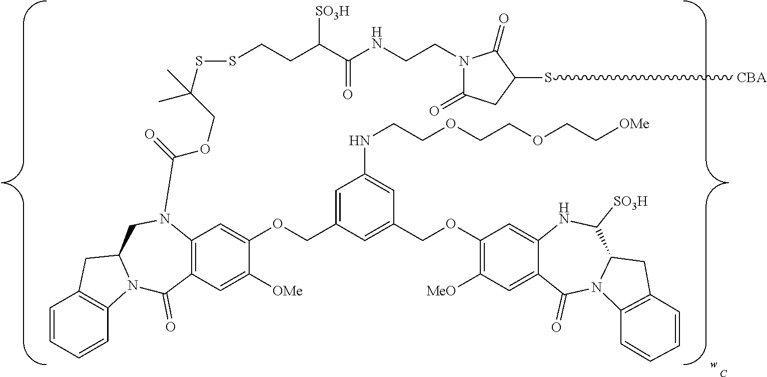

or a pharmaceutically acceptable salt thereof, wherein:

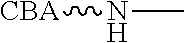

is the cell-binding agent covalently linked to the cytotoxic compound through an amine group located on the CBA;

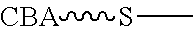

is the cell-binding agent covalently linked to the cytotoxic compound through thiol group located on the CBA;

w_{L} is an integer from 1 to 20;

w_{C} is an integer from 1 to 4; and

the remaining variables are as defined in the second aspect or the 22^{nd} embodiment.

In a 42^{nd} embodiment, for conjugates described above (e.g., conjugates described in the second aspect or any embodiments described therein or in the 22^{nd} to 41^{st} embodiments or any embodiments or specific embodiments described therein), the pharmaceutically acceptable salt thereof is a sodium or potassium salt. In one embodiment, the pharmaceutically acceptable salt is a sodium salt. In one embodiment, the pharmaceutically acceptable salt is a potassium salt.

### Cell-Binding Agents

Cell-binding agents in the immunoconjugates of the present invention can be of any kind presently known, or that become known, including peptides and non-peptides. Generally, these can be antibodies (such as polyclonal antibodies and monoclonal antibodies, especially monoclonal antibodies), lymphokines, hormones, growth factors, vitamins (such as folate etc., which can bind to a cell surface receptor thereof, e.g., a folate receptor), nutrient-transport molecules (such as transferrin), or any other cell-binding molecule or substance.

In certain embodiments, the cell-binding agent is an antibody, a single chain antibody, an antibody fragment that specifically binds to the target cell, a monoclonal antibody, a single chain monoclonal antibody, a monoclonal antibody fragment (or “antigen-binding portion” or “antigen-binding fragment”) that specifically binds to a target cell, a chimeric antibody, a chimeric antibody fragment (or “antigen-binding portion” or “antigen-binding fragment”) that specifically binds to the target cell, a domain antibody (e.g., sdAb), or a domain antibody fragment that specifically binds to the target cell.

In certain embodiments, the cell-binding agent is a humanized antibody, a humanized single chain antibody, or a humanized antibody fragment (or “antigen-binding portion” or “antigen-binding fragment”).

In certain embodiments, the cell-binding agent is a resurfaced antibody, a resurfaced single chain antibody, or a resurfaced antibody fragment (or “antigen-binding portion” or “antigen-binding fragment”).

In certain embodiments, the cell-binding agent is an antibody or an antigen-binding portion thereof (including antibody derivatives), the CBA may bind to a ligand on the target cell, such as a cell-surface ligand, including cell-surface receptors.

In certain embodiments, the cell-binding agent (CBA) binds to target cells selected from tumor cells, virus infected cells, microorganism infected cells, parasite infected cells, autoimmune cells, activated cells, myeloid cells, activated T-cells, B cells, or melanocytes; cells expressing the CA6, CAK1, CD4, CD5, CD6, CD19, CD20, CD22, CD30, CD33, CD37, CD38, CD40, CD44, CD56, CD123, CD138, EpCAM, CanAg, CALLA, CEACAM5, FGFR3, LAMP1, p-cadherin, Her-2 or Her-3 antigens; or cells expressing insulin growth factor receptor, epidermal growth factor receptor, and folate receptor.

In certain embodiments, the cell-binding agent is a cysteine-engineered antibody or antigen-binding fragment thereof. In certain embodiments, the cysteine-engineered antibody or antigen-binding fragment thereof is an anti-folate receptor antibody or an antigen-binding fragment thereof, an anti-EGFR antibody or an antigen-binding fragment thereof, an anti-CD33 antibody or an antigen-binding fragment thereof, an anti-CD19 antibody or an antigen-binding fragment thereof, an anti-Muc1 antibody or an antigen-binding fragment thereof, an anti-CD37 antibody an antigen-binding fragment thereof, anti-cMet antibody or an antigen-binding fragment thereof, or anti-EpCAM antibody or an antigen-binding fragment thereof.

In certain embodiments, the cell-binding agent is an antibody or antigen-binding fragment thereof that: (a) binds an epitope within amino acids 101 to 346 of human CD123/IL3-Rα antigen, and (b) inhibits IL3-dependent proliferation in antigen-positive TF-1 cells (see WO2017/004026, incorporated herein by reference in their entirety).

In certain embodiments, the cell-binding agent is an anti-CD123 antibody or antigen-binding fragment thereof as described in WO2017/004026, which is incorporated herein by reference.

In certain embodiments, the anti-CD123 antibody or antigen-binding fragment thereof may comprise: a) at least one light chain variable region or fragment thereof comprising three sequential complementarity-determining regions (CDR) CDR_{L}1, CDR_{L}2, and CDR_{L}3, respectively, wherein CDR_{L}1 has the amino acid sequence of RASQDINSYLS (SEQ ID NO:1), CDR_{L}2 has the amino acid sequence of RVNRLVD (SEQ ID NO:2), and, CDR_{L}3 has the amino acid sequence of LQYDAFPYT (SEQ ID NO:3); and b) at least one heavy chain variable region or fragment thereof comprising three sequential complementarity-determining regions (CDR) CDR_{H}1, CDR_{H}2, and CDR_{H}3, respectively, wherein, CDR_{H}1 has the amino acid sequence of SSIMH (SEQ ID NO:4), CDR_{H}2 has the amino acid sequence of YIKPYNDGTKYNEKFKG (SEQ ID NO:5), and, CDR_{H}3 has the amino acid sequence of EGGNDYYDTMDY (SEQ ID NO:6).

In certain embodiments, the anti-CD123 antibody or antigen-binding fragment thereof comprises a heavy chain variable region (V_{H}) having the amino acid sequence of

(SEQ ID NO: 7)
QVQLVQSGAEVKKPGASVKVSCKASGYIFT [symbol] WVRQAPGQGLEWIG [symbol] [symbol]
RATLTSDRSTSTAYMELSSLRSEDTAVYYCAR [symbol]
WGQGTLVTVSS

and a light chain variable region (V_{L}) having the amino acid sequence of

(SEQ ID NO: 9)
DIQMTQSPSSLSASVGDRVTITC [symbol] WFQQKPGKAPKTLIY [symbol]
GVPSRFSGSGSGNDYTLTISSLQPEDFATYYC [symbol]
FGQGTKVEIKR.

In certain embodiments, the anti-CD123 antibody has a heavy chain full length sequence of

(SEQ ID NO: 8)
QVQLVQSGAEVKKPGASVKVSCKASGYIFT [symbol] WVRQAPGQGLEWIG [symbol] [symbol]
RATLTSDRSTSTAYMELSSLRSEDTAVYYCAR [symbol]
WGQGTLVTVSSASTKGPSVFPLAPSSKSTSGGTAALGC
LVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLD
SDGSFFLYSKLTVDKSRWQQGNVFSCSVMHEALHNHYTQKSLCLSPG

and a light chain full length sequence of

(SEQ ID NO: 10)
DIQMTQSPSSLSASVGDRVTITC [symbol] WFQQKPGKAPKTLIY [symbol]
GVPSRFSGSGSGNDYTLTISSLQPEDFATYYC [symbol] FG
QGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGL
SSPVTKSFNRGEC

In certain embodiments, the cell-binding agent is an anti-CD33 antibody or an antigen-binding fragment thereof as described in U.S. Pat. Nos. 7,342,110 and 7,557,189, which are incorporated herein by reference.

In certain embodiments, the anti-CD33 antibody or antigen-binding fragment thereof may comprise: a) at least one light chain variable region or fragment thereof comprising three sequential complementarity-determining regions (CDR) CDR_{L}1, CDR_{L}2, and CDR_{L}3, respectively, wherein CDR_{L}1 has the amino acid sequence of KSSQSVFFSSSQKNYLA (SEQ ID NO:11), CDR_{L}2 has the amino acid sequence of WASTRES (SEQ ID NO:12), and, CDR_{L}3 has the amino acid sequence of HQYLSSRT (SEQ ID NO:13); and b) at least one heavy chain variable region or fragment thereof comprising three sequential complementarity-determining regions (CDR) CDR_{H}1, CDR_{H}2, and CDR_{H}3, respectively, wherein, CDR_{H}1 has the amino acid sequence of SYYIH (SEQ ID NO:14), CDR_{H}2 has the amino acid sequence of VIYPGNDDISYNQKFQG (SEQ ID NO:15), and, CDR_{H}3 has the amino acid sequence of EVRLRYFDV (SEQ ID NO:16).

In certain embodiments, the anti-CD33 antibody or antigen-binding fragment thereof comprises a heavy chain variable region (V_{H}) having the amino sequence of

(SEQ ID NO: 17)
QVQLQQPGAEVVKPGASVKMSCKASGYTFT [symbol]
WIKQTPGQGLEWVG [symbol] [symbol]
KATLTADKSSTTAYMQLSSLTSEDSAVYYCAR [symbol]
WGQGTTVTVSS

and a light chain variable region (V_{L}) having the amino acid sequence of

(SEQ ID NO: 19)
EIVLTQSPGSLAVSPGERVTMSC [symbol] W
YQQIPGQSPRLLIY [symbol] [symbol] GVPDRFTGSGSGTDFTLTIS
SVQPEDLAIYYC [symbol] FGQGTKLEIKR.

In certain embodiments, the anti-CD33 antibody has a heavy chain full length sequence of

(SEQ ID NO: 18)
QVQLQQPGAEVVKPGASVKMSCKASGYTFT [symbol] WIKQTPGQGL
EWVG [symbol] [symbol] GKATLTADKSSTTAYMQLSSL
TSEDSAVYYC [symbol] WGQGTTVTVSSASTKGPSVFPLAP
SSKSTSGGTAALGCLVKDYFPEPVTVSWNSGALTSGVHTFPAVL
QSSGLYSLSSVVTVPSSSLGTQTYICNVNHKPSNTKVDKKVEPK
SCDKTHTCPPCPAPELLGGPSVFLFPPKPKDTLMISRTPEVTCV
VVDVSHEDPEVKFNWYVDGVEVHNAKTKPREEQYNSTYRVVSVL
TVLHQDWLNGKEYKCKVSNKALPAPIEKTISKAKGQPREPQVYT
LPPSRDELTKNQVSLTCLVKGFYPSDIAVEWESNGQPENNYKT
TPPVLDSDGSFFLYSKLTVDKSRWQQGNVFSCSVMHEALHNHYT
QKSLSLSPG

and a light chain full length sequence of

(SEQ ID NO: 20)
EIVLTQSPGSLAVSPGERVTMSC [symbol] WY
QQIPGQSPRLLIY [symbol] [symbol] GVPDRFTGSGSGTDFTLTISSV
QPEDLAIYYC [symbol] FGQGTKLEIKRTVAAPSVFIFPPS
DEQLKSGTASVVCLLNNFYPREAKVQWKVDNALQSGNSQESV
TEQDSKDSTYSLSSTLTLSKADYEKHKVYACEVTHQGLSSPV
TKSFNRGEC.

In certain embodiments, the anti-CD33 antibody is huMy9-6 antibody.

In certain embodiment, the cell-binding agent is an anti-ADAM9 antibody or an antigen-binding fragment thereof as described in WO2018/119196 and U.S. Provisional Application Nos. 62/690,052 and 62/691,342, each of which are incorporated herein by reference.

In certain embodiments, the anti-ADAM9 antibody or antigen-binding fragment thereof is a humanized anti-ADAM9 antibody or antigen-binding fragment thereof that specifically binds to human ADAM9 and cyno ADAM9.

In certain embodiments, the humanized anti-ADAM9 antibody or ADAM9-binding fragment thereof is optimized to have at least a 100-fold enhancement in binding affinity to cyno ADAM9 and retains high affinity binding to human ADAM9 as compared to the chimeric or murine parental antibody.

In certain embodiments, the anti-ADAM9 antibody or antigen-binding fragment thereof (e.g., the humanized anti-ADAM9 antibody or antigen-binding fragment thereof) comprises: a) at least one light chain variable region or fragment thereof comprising three sequential complementarity-determining regions (CDR) CDR_{L}1, CDR_{L}2, and CDR_{L}3, respectively, wherein CDR_{L}1 has the amino acid sequence of KASQSVDYSGDSYMN (SEQ ID NO:21), CDR_{L}2 has the amino acid sequence of AASDLES (SEQ ID NO:22), and, CDR_{L}3 has the amino acid sequence of QQSHEDPFT (SEQ ID NO:23); and b) at least one heavy chain variable region or fragment thereof comprising three sequential complementarity-determining regions (CDR) CDR_{H}1, CDR_{H}2, and CDR_{H}3, respectively, wherein, CDR_{H}1 has the amino acid sequence of SYWMH (SEQ ID NO:24), CDR_{H}2 has the amino acid sequence of EIIPIFGHTNYNEKFKS (SEQ ID NO:25), and, CDR_{H}3 has the amino acid sequence of GGYYYYPRQGFLDY (SEQ ID NO:26).

In certain embodiments, the anti-ADAM9 antibody or antigen-binding fragment thereof (e.g., the humanized anti-ADAM9 antibody or antigen-binding fragment thereof) comprises a heavy chain variable region (V_{H}) having the amino sequence of

(SEQ ID NO: 27)
EVQLVESGGG LVKPGGSLRLSCAASGFTFS [symbol]
WVRQA PGKGLEWVG [symbol] [symbol]
RFTI SLDNSKNTLY
LQMGSLRAED TAVYYCA [symbol] [symbol]
WGQGTTVT VSS

and a light chain variable region (V_{L}) having the amino acid sequence of

(SEQ ID NO: 28)
DIVMTQSPDSLAVSLGERATISC [symbol]
YQQKPGQPPKLLIY [symbol] [symbol] GIPARFSGSG
SGTDFTLTIS SLEPEDFATYYC [symbol]
FGQGTKLEI K.

In certain embodiments, the anti-ADAM9 antibody has a heavy chain full length sequence of

(SEQ ID NO: 29)
EVQLVESGGG LVKPGGSLRL SCAASGFTFS [symbol] WVRQA
PGKGLEWVG [symbol] [symbol] RFTI SLDNSKNTLY
LQMGSLRAED TAVYYCAR [symbol] [symbol] WGQGTTVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKR VEPKSCDKTH TCPPCPAPEL
LGGPSVFLFP PKPKDTL [symbol] R [symbol] PEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPS
REEMTKNQVS LTCLVKGFYP SDIAVEWESN GQPENNYKTT
PPVLDSDGSF FLYSKLTVDK SRWQQGNVFS CSVMHEALHN
HYTQKSLCLS PG

and a light chain full length sequence of

(SEQ ID NO: 30)
DIVMTQSPDSLAVSLGERATISC [symbol] WYQQK
PGQPPKLLIY [symbol] [symbol] GIPARFSGSGSGTDFTLTISSLEPEDF
ATYYC [symbol] FGQGTKLEIKRTVAAPSVFIFPPSDEQLKS
GTASVVCLLNNFYPREAKVQWKVDNALQSGNSQESVTEQDSKDST
YSLSSTLTLSKADYEKHKVYACEVTHQGLSSPVTKSFNRGEC.

In certain embodiment, the cell-binding agent is an anti-EpCAM antibody or an antigen-binding fragment thereof as described in U.S. Provisional Application No. 62/751,530, incorporated herein by reference.

In certain embodiments, the anti-EpCAM antibody or antigen-binding fragment thereof may comprise: a) at least one light chain variable region or fragment thereof comprising three sequential complementarity-determining regions (CDR) CDR_{L}1, CDR_{L}2, and CDR_{L}3, respectively, wherein CDR_{L}1 has the amino acid sequence of RSSRSLLHSDGFTYLY (SEQ ID NO:31), CDR_{L}2 has the amino acid sequence of QTSNLAS (SEQ ID NO:32), and, CDR_{L}3 has the amino acid sequence of AQNLELPNT (SEQ ID NO:33); and b) at least one heavy chain variable region or fragment thereof comprising three sequential complementarity-determining regions (CDR) CDR_{H}1, CDR_{H}2, and CDR_{H}3, respectively, wherein, CDR_{H}1 has the amino acid sequence of NYYIH (SEQ ID NO:34), CDR_{H}2 has the amino acid sequence of WIYPGNVYIQYNEKFKG (SEQ ID NO:35), and, CDR_{H}3 has the amino acid sequence of DGPWFAY (SEQ ID NO:36).

In certain embodiments, the anti-EpCAM antibody or antigen-binding fragment thereof comprises a heavy chain variable region (V_{H}) having the amino sequence of

(SEQ ID NO: 37)
QVQLVQSGAEVKKPGASVKVSCKASGYTFT [symbol] W
VRQAPGQRLEYIG [symbol] [symbol] R
ATLTADKSASTAYMELSSLRSEDTAVYYCAR [symbol]
WGQGTLVTVSS

and a light chain variable region (V_{L}) having the amino acid sequence of

(SEQ ID NO: 38)
DIVLTQTPLSLSVTPGQPASISC [symbol]
WFLQKPGQSPQLLIY [symbol] [symbol] GVPDRFSSSGSGTDFT
LKISRVEAEDVGVYYC [symbol] FGQGTKLEIK.

In certain embodiments, the anti-EpCAM antibody has a heavy chain full length sequence of

(SEQ ID NO: 39)
QVQLVQSGAEVKKPGASVKVSCKASGYTFT [symbol] M
DMWVRQAPGQRLEYIG [symbol] [symbol] RATLTA
DKSASTAYMELSSLRSEDTAVYYCAR [symbol] WGQG
TLVTVSSASTKGPSVFPLAPSSKSTSGGTAALGCLVKDY
FPEPVTVSWNSGALTSGVHTFPAVLQSSGLYSLSSVVTV
PSSSLGTQTYICNVNHKPSNTKVDKKVEPKSCDKTHTCP
PCPAPELLGGPSVFLFPPKPKDTLMISRTPEVTCVVVDV
SHEDPEVKFNWYVDGVEVHNAKTKPREEQYNSTYRVVSV
LTVLHQDWLNGKEYKCKVSNKALPAPIEKTISKAKGQPR
EPQVYTLPPSRDELTKNQVSLTCLVKGFYPSDIAVEWES
NGQPENNYKTTPPVLDSDGSFFLYSKLTVDKSRWQQGNV
FSCSVMHEALHNHYTQKSLSLSPG

and a light chain full length sequence of

(SEQ ID NO: 40)
DIVLTQTPLSLSVTPGQPASISC [symbol] L
YWFLQKPGQSPQLLIY [symbol] [symbol] GVPDRFSSS
GSGTDFTLKISRVEAEDVGVYYC [symbol]
FGQGTKLEIKRTVAAPSVFIFPPSDEQLKSGT
ASVVCLLNNFYPREAKVQWKVDNALQSGNSQE
SVTEQDSKDSTYSLSSTLTLSKADYEKHKVYA
CEVTHQGLSSPVTKSFNRGEC.

In certain embodiments, the cell-binding agent is an anti-folate receptor antibody. In certain embodiments, the cell-binding agent is an anti-human folate receptor 1 (FOLR1) antibody or an antigen-binding fragment thereof as described in U.S. Pat. Nos. 8,709,432, 8,557,966, and WO2011106528, all of which are incorporated herein by reference.

In certain embodiments, the anti-FOLR1 antibody or antigen-binding fragment thereof may comprise: a) at least one light chain variable region or fragment thereof comprising three sequential complementarity-determining regions (CDR) CDR_{L}1, CDR_{L}2, and CDR_{L}3, respectively, wherein CDR_{L}1 has the amino acid sequence of KASQSVSFAGTSLMH (SEQ ID NO:41), CDR_{L}2 has the amino acid sequence of RASNLEA (SEQ ID NO:42), and, CDR_{L}3 has the amino acid sequence of QQSREYPYT (SEQ ID NO:43); and b) at least one heavy chain variable region or fragment thereof comprising three sequential complementarity-determining regions (CDR) CDR_{H}1, CDR_{H}2, and CDR_{H}3, respectively, wherein, CDR_{H}1 has the amino acid sequence of GYFMN (SEQ ID NO:44) or GYTFTGYFMN (SEQ ID NO:47), CDR_{H}2 has the amino acid sequence of RIHPYDGDTFYNQKFQG (SEQ ID NO:45) or RIHPYDGDTF (SEQ ID NO:48), and, CDR_{H}3 has the amino acid sequence of YDGSRAMDY (SEQ ID NO:46). In certain embodiments, the anti-FOLR1 antibody or antigen-binding fragment thereof comprises a) a light chain variable region comprising a CDR_{L}1 having an amino sequence set forth in SEQ ID NO:41, a CDR_{L}2 having an amino sequence set forth in SEQ ID NO:42, and a CDR_{L}3 having an amino sequence set forth in SEQ ID NO:43; and b) a heavy chain variable region comprising a CDR_{H}1 having an amino sequence set forth in SEQ ID NO:44, a CDR_{H}2 having an amino sequence set forth in SEQ ID NO:45, and a CDR_{H}3 having an amino sequence set forth in SEQ ID NO:46. In certain embodiments, the anti-FOLR1 antibody or antigen-binding fragment thereof comprises a) a light chain variable region comprising a CDR_{L}1 having an amino sequence set forth in SEQ ID NO:41, a CDR_{L}2 having an amino sequence set forth in SEQ ID NO:42, and a CDR_{L}3 having an amino sequence set forth in SEQ ID NO:43; and b) a heavy chain variable region comprising a CDR_{H}1 having an amino sequence set forth in SEQ ID NO:47, a CDR_{H}2 having an amino sequence set forth in SEQ ID NO:48, and a CDR_{H}3 having an amino sequence set forth in SEQ ID NO:46.

In certain embodiments, the anti-FOLR1 antibody or antigen-binding fragment thereof comprises a heavy chain variable region V_{H} having the amino sequence of

(SEQ ID NO: 49)
QVQLVQSGAEVVKPGASVKISCKAS [symbol] W
VKQSPGQSLEWIG [symbol] [symbol] KATLT
VDKSSNTAHMELLSLTSEDFAVYY [symbol] Y
WGQGTTVTVSS

and a light chain variable region (V_{L}) having the amino acid sequence of

(SEQ ID NO: 50)
DIVLTQSPLSLAVSLGQPAIISC [symbol] WY
HQKPGQQPRLLIY [symbol] [symbol] GVPDRFSGSGSKTDFTLNISP
VEAEDAATYYC [symbol] FGGGTKLEIKR,
or
(SEQ ID NO: 51)
DIVLTQSPLSLAVSLGQPAIISC [symbol] W
YHQKPGQQPRLLIY [symbol] [symbol] GVPDRFSGSGSKTDFTLTI
SPVEAEDAATYYC [symbol] FGGGTKLEIKR.

In certain embodiments, the anti-FOLR1 antibody has a heavy chain full length sequence of

(SEQ ID NO: 52)
QVQLVQSGAEVVKPGASVKISCKAS [symbol] W
VKQSPGQSLEWIG [symbol] [symbol] KATLT
VDKSSNTAHMELLSLTSEDFAVYY [symbol] W
GQGTTVTVSSASTKGPSVFPLAPSSKSTSGGTAALG
CLVKDYFPEPVTVSWNSGALTSGVHTFPAVLQSSGL
YSLSSVVTVPSSSLGTQTYICNVNHKPSNTKVDKKV
EPKSCDKTHTCPPCPAPELLGGPSVFLFPPKPKDTL
MISRTPEVTCVVVDVSHEDPEVKFNWYVDGVEVHNA
KTKPREEQYNSTYRVVSVLTVLHQDWLNGKEYKCKV
SNKALPAPIEKTISKAKGQPREPQVYTLPPSRDELT
KNQVSLTCLVKGFYPSDIAVEWESNGQPENNYKTTP
PVLDSDGSFFLYSKLTVDKSRWQQGNVFSCSVMHEA
LHNHYTQKSLSLSPGK

and a light chain full length sequence of

(SEQ ID NO: 53)
DIVLTQSPLSLAVSLGQPAIISC [symbol] WYHQKPGQQPRLL
IY [symbol] [symbol] GVPDRFSGSGSKTDFTLNISPVEAEDAATYYC [symbol]
FGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGEC,
or
(SEQ ID NO: 54)
DIVLTQSPLSLAVSLGQPAIISC [symbol] WYHQKPGQQPRLL
IY [symbol] [symbol] GVPDRFSGSGSKTDFTLTISPVEAEDAATYYC [symbol]
FGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGEC.

In certain embodiments, the anti-FOLR1 antibody is huMov19 or M9346A antibody.

In certain embodiments, the antibody described herein is a murine, non-human mammal, chimeric, humanized, or human antibody. For example, the humanized antibody may be a CDR-grafted antibody or resurfaced antibody. In certain embodiments, the antibody is a full-length antibody. In certain embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In certain embodiments, the cell-binding agent is an alternative protein scaffold, such as a Centyrin (a protein scaffold based on a consensus sequence of fibronectin type III (FN3) repeats; see U.S. Patent Publication 2010/0255056, 2010/0216708 and 2011/0274623 incorporated herein by reference), an Ankyrin Repeat Protein (e.g., a designed ankyrin repeat protein, known as DARPin; see U.S. Patent Publication Nos. 2004/0132028, 2009/0082274, 2011/0118146, and 2011/0224100, incorporated herein by reference, and also see C. Zahnd et al., Cancer Res. (2010) 70:1595-1605; Zahnd et al., J. Biol. Chem. (2006) 281(46):35167-35175; and Binz, H. K., Amstutz, P. & Pluckthun, A., Nature Biotechnology (2005) 23:1257-1268, incorporated herein by reference), an ankyrin-like repeats protein or synthetic peptide (see e.g., U.S. Patent Publication No. 2007/0238667; U.S. Pat. No. 7,101,675; WO 2007/147213; and WO 2007/062466, incorporated herein by reference), an Adnectin (a fibronectin domain scaffold protein; see US Patent Publication Nos. 2007/0082365; 2008/0139791, incorporated herein by reference), Avibody (including diabodies, triabodies, and tetrabodies; see U.S. Publication Nos. 2008/0152586 and 2012/0171115), dual receptor retargeting (DART) molecules (P. A. Moore et al., Blood, 2011; 117(17):4542-4551; Veri MC, et al., Arthritis Rheum, 2010 Mar. 30; 62(7):1933-43; Johnson S, et al. J Mol Biol, 2010 Apr. 9; 399(3):436-49), and cell penetrating supercharged proteins (Methods in Enzymol. 502, 293-319 (2012).

### Production of Cell-Binding Agent-Drug Conjugates

In order to link the cytotoxic compounds or derivative thereof of the present invention to the cell-binding agent, the cytotoxic compound can comprise a linking moiety with a reactive group bonded thereto. These compounds can be directly linked to the cell-binding agent. Representative processes for linking the cytotoxic compounds having a reactive group bonded thereof with the cell-binding agent to produce the cell-binding agent-cytotoxic agent conjugates are described in Example s32-36.

In some embodiments, a bifunctional crosslinking reagent can be first reacted with the cytotoxic compound to provide the compound bearing a linking moiety with one reactive group bonded thereto (i.e., drug-linker compound), which can then react with a cell binding agent. Alternatively, one end of the bifunctional crosslinking reagent can first react with the cell binding agent to provide the cell binding agent bearing a linking moiety with one reactive group bonded thereto, which can then react with a cytotoxic compound. The linking moiety can contain a chemical bond that allows for the release of the cytotoxic moiety at a particular site. Suitable chemical bonds are well known in the art and include disulfide bonds, thioether bonds, acid labile bonds, photolabile bonds, peptidase labile bonds and esterase labile bonds (see for example U.S. Pat. Nos. 5,208,020; 5,475,092; 6,441,163; 6,716,821; 6,913,748; 7,276,497; 7,276,499; 7,368,565; 7,388,026 and 7,414,073). Preferred are disulfide bonds, thioether and peptidase labile bonds. Other linkers that can be used in the present invention include non-cleavable linkers, such as those described in are described in detail in U.S. publication number 2005/0169933, or charged linkers or hydrophilic linkers and are described in US 2009/0274713, US 2010/01293140 and WO 2009/134976, each of which is expressly incorporated herein by reference, each of which is expressly incorporated herein by reference.

In some embodiments, a solution of a cell-binding agent (e.g., an antibody) in aqueous buffer may be incubated with a molar excess of a bifunctional crosslinking agent, such as N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB) to introduce dithiopyridyl groups. The modified cell-binding agent (e.g., modified antibody) is then reacted with the thiol-containing cytotoxic compound described herein, to produce a disulfide-linked cell-binding agent-cytotoxic agent conjugate of the present invention.

In another embodiment, the thiol-containing cytotoxic compound described herein, can react with a bifunctional crosslinking agent such as N-succinimidyl-4-(2-pyridyldithio)pentanoate (SPP), N-succinimidyl-4-(2-pyridyldithio)butanoate (SPDB), N-succinimidyl-4-(2-pyridyldithio)2-sulfo butanoate (sulfo-SPDB) to form a cytotoxic agent-linker compound, which can then react with a cell-biding agent to produce a disulfide-linked cell-binding agent-cytotoxic agent conjugate of the present invention. The cytotoxic agent-linker compound can be prepared in situ without purification before reacting with the cell-binding agent. Alternatively, the cytotoxic agent-linker compound can be purified prior to reacting with the cell-binding agent.

The cell binding agent-cytotoxic agent conjugate may be purified using any purification methods known in the art, such as those described in U.S. Pat. No. 7,811,572 and US Publication No. 2006/0182750, both of which are incorporated herein by reference. For example, the cell-binding agent-cytotoxic agent conjugate can be purified using tangential flow filtration, adsorptive chromatography, adsorptive filtration, selective precipitation, non-absorptive filtration or combination thereof. Preferably, tangential flow filtration (TFF, also known as cross flow filtration, ultrafiltration and diafiltration) and/or adsorptive chromatography resins are used for the purification of the conjugates.

The number of cytotoxic molecules bound per antibody molecule can be determined spectrophotometrically by measuring the ratio of the absorbance at 280 nm and 330 nm. In some embodiments, an average of 1-10 cytotoxic compounds/antibody molecule(s) can be linked by the methods described herein. In some embodiments, the average number of linked cytotoxic compounds per antibody molecule (DAR) is 2-5, and more specifically 2.5-4.0. In some embodiment, a composition (e.g., pharmaceutical composition) comprising the conjugates of the invention has a DAR value between 2 and 8, 2 and 5, more specifically between 2.5 and 4.0.

In some embodiments, when the antibody is linked to the cytotoxic agent through a cysteine thiol group, the conjugate has 1 to 4 cytotoxic compounds per antibody molecule. In some embodiments, the conjugate has 1 or 2 cytotoxic compounds per antibody molecule. In some embodiments, the conjugate has 2 cytotoxic compounds per antibody molecule. In some embodiments, the average number of linked cytotoxic compounds per antibody molecule (DAR) is 1.5 to 2.5, more specifically 1.8-2.2. In some embodiments, a composition (e.g., pharmaceutical composition) comprising the conjugates of the invention has a DAR value between 1.0 and 2.5, between 1.5 and 2.5, more specifically between 1.8 and 2.2 or between 1.9 and 2.1.

Representative processes for preparing the cell-binding agent-drug conjugates of the present invention are described in 8,765,740 and U.S. Application Publication No. 2012/0238731. The entire teachings of these references are incorporated herein by reference.

### Compositions and Methods of Use

The present invention includes a composition (e.g., a pharmaceutical composition) comprising the cytotoxic compounds described herein, derivatives thereof, or conjugates thereof, (and/or solvates, hydrates and/or salts thereof) and a carrier (a pharmaceutically acceptable carrier).

The pharmaceutical compositions described herein can be administered in any number of ways for either local or systemic treatment. Administration can be topical (such as to mucous membranes including vaginal and rectal delivery) such as transdermal patches, ointments, lotions, creams, gels, drops, suppositories, sprays, liquids and powders; pulmonary (e.g., by inhalation or insufflation of powders or aerosols, including by nebulizer; intratracheal, intranasal, epidermal and transdermal); oral; or parenteral including intravenous, intraarterial, subcutaneous, intraperitoneal or intramuscular injection or infusion; or intracranial (e.g., intrathecal or intraventricular) administration. In some particular embodiments, the administration is intravenous. The pharmaceutical compositions described herein can also be used in vitro or in ex vivo.

The present compositions are useful for inhibiting abnormal cell growth or treating a proliferative disorder in a mammal (e.g., human).

The present invention includes a method of inhibiting abnormal cell growth or treating a proliferative disorder, an autoimmune disorder, destructive bone disorder, infectious disease, viral disease, fibrotic disease, neurodegenerative disorder, pancreatitis or kidney disease in a mammal (e.g., human) comprising administering to said mammal a therapeutically effective amount of cytotoxic compounds described herein, derivatives thereof, or conjugates thereof, (and/or solvates and salts thereof) or a composition thereof.

In certain embodiments, the proliferative disorder in a mammal is cancer, including hematologic cancer, leukemia, or lymphoma. In certain embodiments, the proliferative disorder is a cancer of a lymphatic organ, or a hematological malignancy.

For example, the cancer may be selected from the group consisting of: acute myeloid leukemia (AML, including CD33-low AML, P-glycoprotein positive AML, relapsed AML, or refractory AML), chronic myelogenous leukemia (CML), including blastic crisis of CML and Abelson oncogene associated with CML (Bcr-ABL translocation), myelodysplastic syndrome (MDS), acute lymphoblastic leukemia (ALL), including, but not limited to, acute B lymphoblastic leukemia or B-cell acute lymphoblastic leukemia (B-ALL), chronic lymphocytic leukemia (CLL), including Richter's syndrome or Richter's transformation of CLL, hairy cell leukemia (HCL), acute promyelocytic leukemia (APL), B-cell chronic lymphoproliferative disease (B-CLPD), atypical chronic lymphocytic leukemia (preferably with a marked CD11c expression), diffuse large B-cell lymphoma (DLBCL), blastic plasmacytoid dendritic cell neoplasm (BPDCN), non-Hodgkin lymphomas (NHL), including mantel cell leukemia (MCL), and small lymphocytic lymphoma (SLL), Hodgkin's lymphoma, systemic mastocytosis, and Burkitt's lymphoma.

In certain embodiments, the cancer may be selected form the group consisting of lung cancer (e.g., non-small-cell lung cancer), colorectal cancer, bladder cancer, gastric cancer, pancreatic cancer, renal cell carcinoma, prostate cancer, esophageal cancer, breast cancer, head and neck cancer, uterine cancer, ovarian cancer, liver cancer, cervical cancer, thyroid cancer, testicular cancer, myeloid cancer, melanoma, and lymphoid cancer. In certain embodiments, the cancer is non-small-cell lung cancer, colorectal cancer, gastric cancer, breast cancer (e.g., triple negative breast cancer (TNBC)) or pancreatic cancer. In further embodiments, immunoconjugates of the present invention may be useful in the treatment of non-small-cell lung cancer (squamous cell, nonsquamous cell, adenocarcinoma, or large-cell undifferentiated carcinoma), colorectal cancer (adenocarcinoma, gastrointestinal carcinoid tumors, gastrointestinal stromal tumors, primary colorectal lymphoma, leiomyosarcoma, or squamous cell carcinoma) or breast cancer (e.g., triple negative breast cancer (TNBC))

Suitable pharmaceutically acceptable carriers, diluents, and excipients are well known and can be determined by those of ordinary skill in the art as the clinical situation warrants. Examples of suitable carriers, diluents and/or excipients include: (1) Dulbecco's phosphate buffered saline, pH about 7.4, containing or not containing about 1 mg/mL to 25 mg/mL human serum albumin, (2) 0.9% saline (0.9% w/v NaCl), and (3) 5% (w/v) dextrose; and may also contain an antioxidant such as tryptamine and a stabilizing agent such as Tween 20.

### Exemplification

### Example 1. Synthesis of Compound 18 and Compound 19

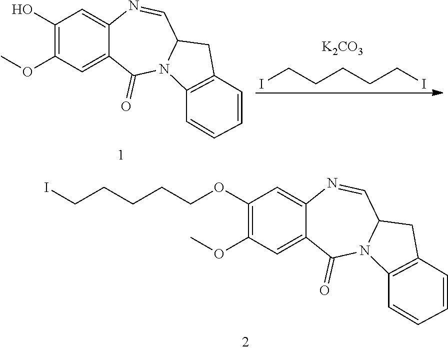

Compound 1 (1 g, 3.40 mmol) was dissolved in dimethylformamide (22.65 ml). 1,5-Diiodopentane (3.66 ml, 23.78 mmol) and potassium carbonate (1.174 g, 8.49 mmol) were added. The reaction was protected from light and stirred at room temperature overnight. The reaction was diluted with methylene chloride and washed with aqueous ammonium chloride and brine. The organic layer was dried over anhydrous sodium sulfate, filtered, and concentrated in vacuo. The crude material was purified by silica gel chromatography in ethyl acetate/dichloromethane to yield compound 2 (1.05 g, y=63%) MS (m/z): 491.1 (M+1)+. UPLC=4.93 min (10 min method).

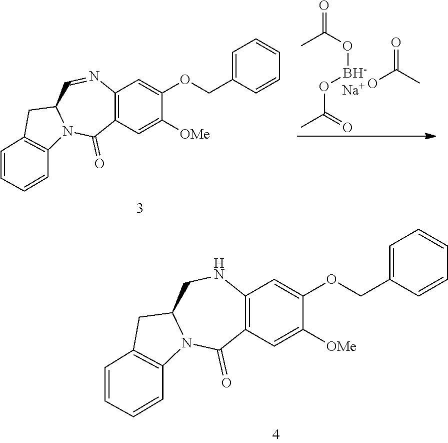

(S)-9-(benzyloxy)-8-methoxy-12a,13-dihydro-6H-benzo[5,6][1,4]diazepino[1,2-a]indol-6-one (4.3 g, 11.19 mmol) was dissolved in anhydrous 1,2-Dichloroethane (102 ml) under nitrogen and sodium triacetoxyborohydride (7.11 g, 33.6 mmol) was added. The reaction stirred at room temperature for 4 hours. The mixture was cooled to 0° C. and quenched with saturated ammonium chloride and then extracted with dichloromethane. The combined organics were washed with brine, dried with anhydrous magnesium sulfate, filtered and concentrated. The crude compound 4 was carried on without further purification, assuming 100% yield. MS (m/z): 387.2 (M+1)⁺. UPLC=1.65 min (2.5 min method). (UPLC 2.5 min method) Analytical BEH Phenyl HPLC Method:

| Column: | Acquity UPLC BEH- C18 2.1 × 50 mm, 1.7 μm | |
|---|---|---|
| | particle size, P/N 186002350, SN 02743604615173 | |
| Flow rate: | 0.8 mL/min | |
| Temperature: | Ambient | |
| Mobile phase A: | deionized water + 0.1% formic acid | |
| Mobile phase B: | acetonitrile | |
| Gradient: | Time | % B |
| | 0 | 5 |
| | 1.8 | 98 |
| | 2 | 98 |
| | 2.1 | 5 |
| | 2.5 | 5 |

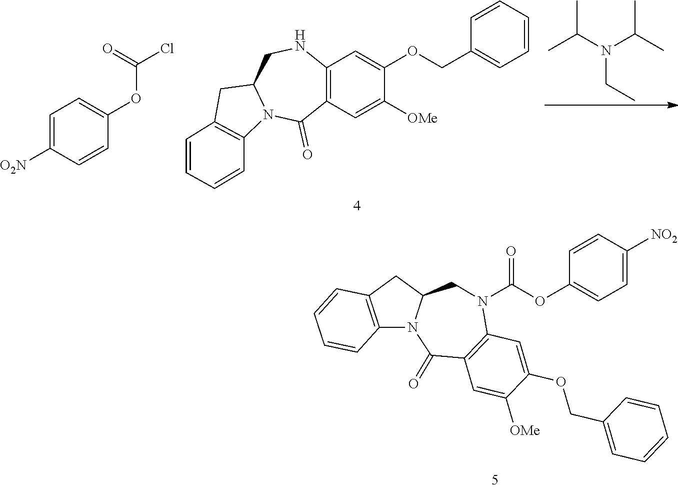

To a solution of Compound 4 (4.32 g, 11.19 mmol) was added Hunig's base (2.339 ml, 13.43 mmol) followed by a solution of 4-nitrophenyl carbonochloridate (2.58 g, 12.31 mmol) in dichloromethane at room temperature. After 4 hours the reaction was quenched with water, and layers separated. The organic layer was washed with water, saturated sodium bicarbonate, and brine. It was dried over magnesium sulfate, filtered and stripped. The crude solid was purified by silica gel chromatography in ethyl acetate/hexanes to give Compound 5 as a pale yellow fluffy solid (4.4 g, y=71%) MS (m/z): 552.5 (M+1)⁺. UPLC=1.87 min (2.5 min method).

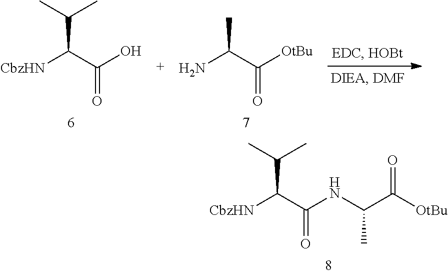

Compound 6 (3.0 g, 11.94 mmol) and Compound 7 (1.907 g, 13.13 mmol) were dissolved in dimethylformamide (23.88 mL). EDC-HCl (2.52 g, 13.13 mmol) and HOBt (2.011 g, 13.13 mmol) were added to the reaction mixture, followed by DIEA (4.59 mL, 26.3 mmol). The reaction was stirred at room temperature overnight under Ar. The reaction mixture was diluted with dichloromethane and was washed with saturated sodium bicarbonate, saturated ammonium chloride, water and brine. The organic layer was dried over sodium sulfate, filtered and concentrated. The crude residue was purified by silica gel flash chromatography (ethyl acetate/hexanes) to obtain Compound 8 as a white solid (3.68 g, 81% yield). ¹H NMR (400 MHz, CDCl₃): δ 7.39-7.29 (m, 5H), 6.29 (bd, 1H, J=6.9 Hz), 5.34 (bd, 1H, J=8.4 Hz), 5.11 (s, 2H), 4.45 (p, 1H, J=7.2 Hz), 4.02-3.98 (m, 1H), 2.18-2.09 (m, 1H), 1.56 (s, 9H), 1.37 (d, 3H, J=7.0 Hz), 0.98 (d, 3H, J=6.8 Hz), 0.93 (d, 3H, J=6.8 Hz). LCMS=5.571 min (8 min method). Mass observed (ESI⁺): 323.25 (M-tBu+H).

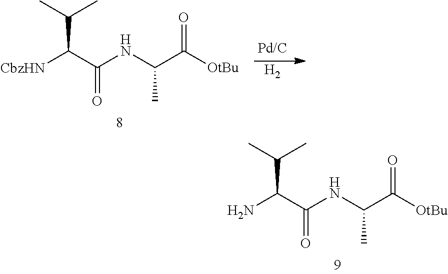 [SMILES: CC(C)[C@H](NC(=O)OCc1ccccc1)C(=O)N[C@@H](C)C(=O)OC(C)(C)C ; "H2" ; CC(C)[C@H](N)C(=O)N[C@@H](C)C(=O)OC(C)(C)C]

Compound 8 (3.68 g, 9.72 mmol) was dissolved in methanol (30.9 mL) and water (1.543 mL). The solution was purged with Ar and was degassed for 5 minutes. Pd/C (10%, wet, 0.517 g) was added slowly to the reaction mixture. H₂ was then bubbled in for a minute. Bubbling was discontinued and the reaction was then stirred under a H₂ balloon overnight. The reaction mixture was filtered through Celite and the filter cake was washed with methanol (30 mL) and was concentrated to obtain compound 9 as a white solid (2.35 g, 99% yield). ¹H NMR (400 MHz, CDCl₃): δ 7.79-7.77 (m, 1H), 4.50 (p, 1H, J=7.3 Hz), 3.27 (d, 1H, J=3.9 Hz), 2.34-2.26 (m, 1H), 1.49 (s, 9H), 1.40 (d, 3H, J=7.1 Hz), 1.01 (d, 3H, J=7.0 Hz), 0.86 (d, 3H, J=6.9 Hz).

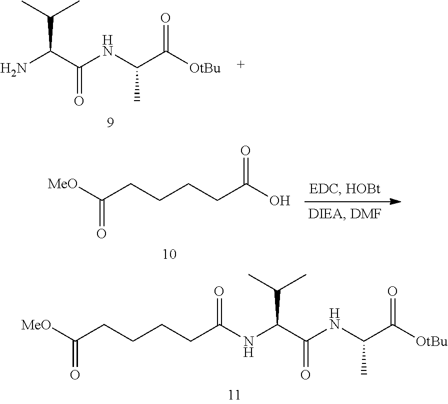 [SMILES: CC(C)[C@H](N)C(=O)N[C@@H](C)C(=O)OC(C)(C)C ; COC(=O)CCCCC(=O)O ; COC(=O)CCCCC(=O)N[C@H](C(=O)N[C@@H](C)C(=O)OC(C)(C)C)C(C)C]

Compound 9 (2.35 g, 9.62 mmol) and mono methyladipate (1.69 g, 10.58 mmol) were dissolved in dimethylformamide (32.1 mL). EDC-HCl (1.94 g, 10.10 mmol) and HOBt (1.47 g, 9.62 mmol) were added to the reaction mixture, followed by DIEA (3.36 mL, 19.24 mmol). The reaction was stirred at room temperature overnight. The reaction mixture was diluted with dichloromethane/methanol (20 mL, 5:1) and was washed with saturated ammonium chloride, saturated sodium bicarbonate, water and brine. The organic layer was dried over sodium sulfate, filtered and concentrated. The crude product was purified by silica gel flash chromatography (ethyl acetate/hexanes, gradient, 0% to 50%) to obtain compound 11 as a white solid (2.77 g, 75% yield). ¹H NMR (400 MHz, CDCl₃): δ 6.29 (d, 1H, J=7.2 Hz), 6.12 (d, 1H, J=8.6 Hz), 4.43 (p, 1H, J=7.2 Hz), 4.27 (dd, 1H, J=6.4, 8.6 Hz), 3.66 (s, 3H), 2.35-2.31 (m, 2H), 2.26-2.23 (m, 2H), 2.12-2.03 (m, 1H), 1.70-1.63 (m, 4H), 1.46 (s, 9H), 1.36 (d, 3H, J=7.1 Hz), 0.95 (apparent t, 6H, J=6.6 Hz).

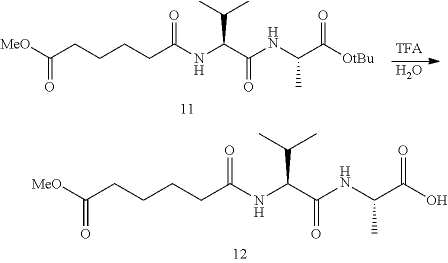 [SMILES: COC(=O)CCCCC(=O)N[C@H](C(=O)N[C@@H](C)C(=O)OC(C)(C)C)C(C)C ; O ; COC(=O)CCCCC(=O)N[C@H](C(=O)N[C@@H](C)C(=O)O)C(C)C]

TFA (8.28 mL, 108.0 mmol) and water (0.56 mL) were added to neat compound 11 (2.77 g, 7.17 mmol) at room temperature and was stirred for 2.5 h. acetonitrile (30 mL) was added to the reaction mixture and was concentrated. This was repeated 2 more times to obtain compound 12 as a pale yellow solid (2.0 g, 84% yield). ¹H NMR (400 MHz, CDCl₃): δ 8.11 (bs, 1H), 7.29 (d, 1H, J=8.9 Hz), 7.14 (d, 1H, 6.8 Hz), 4.58 (p, 1H, J=7.1 Hz), 4.37 (t, 1H, J=8.7 Hz), 3.68 (s, 3H), 2.37-2.32 (m, 4H), 2.03-1.99 (m, 2H), 1.69-1.63 (m, 4H), 1.49 (d, 3H, J=7.2 Hz), 0.97 (d, 3H, J=4.8 Hz), 0.96 (d, 3H, J=4.8 Hz).

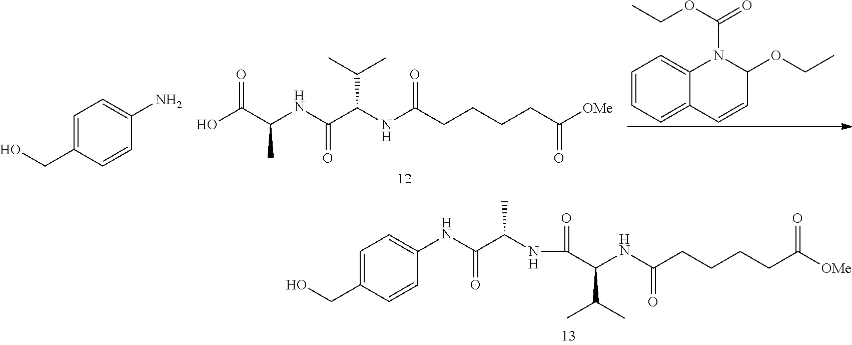

(6-methoxy-6-oxohexanoyl)-L-valyl-L-alanine (4.93 g, 14.92 mmol) was suspended in anhydrous dichloromethane (49.7 ml) and anhydrous methanol (24.87 ml). EEDQ (6.64 g, 26.9 mmol) and then (4-aminophenyl)methanol (2.205 g, 17.91 mmol) were added and the reaction was stirred overnight at room temperature. The reaction mixture was concentrated and coevaporated with dichloromethane. The crude material was purified by silica gel chromatography in dichloromethane/methanol to give Compound 13 (2.04 g, y=31%) MS (m/z): 434.3 (M−1)⁻. UPLC=1.23 min (2.5 min method).

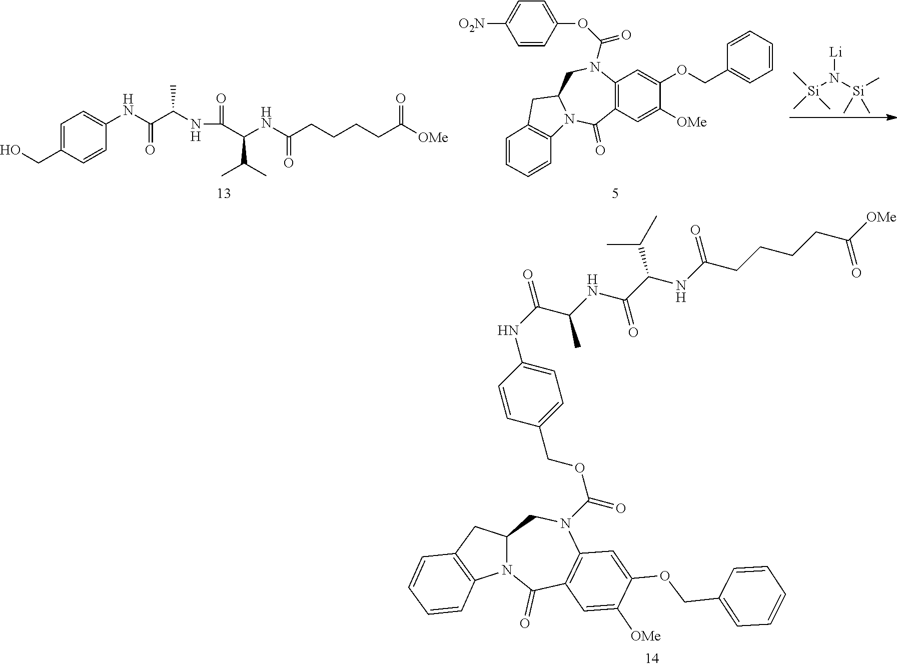

To a solution of compound 13 (805 mg, 1.848 mmol) in anhydrous THF (3501 μl) and anhydrous dimethylacetamide (7001 μl) was added lithium bis(trimethylsilyl)amide (1M in THF) (1848 μl, 1.848 mmol) at OC. The reaction was stirred for 15 minutes before compound 5 (850 mg, 1.540 mmol) in anhydrous THF (3501 μl) was added. The reaction mixture was stirred at 0 C and allowed to warm to room temperature overnight. The reaction was quenched at 0 C with saturated ammonium chloride causing a precipitate to form. The solution was extracted with dichloromethane. The combined organics were washed with water (2×). The organic was dried with magnesium sulfate, filtered and concentrated. It was purified by silica gel chromatography (dichloromethane/methanol) to give compound 14 (430 mg, y=33%). MS (m/z): 848.7 (M+1)⁺. UPLC=1.74 min (2.5 min method).

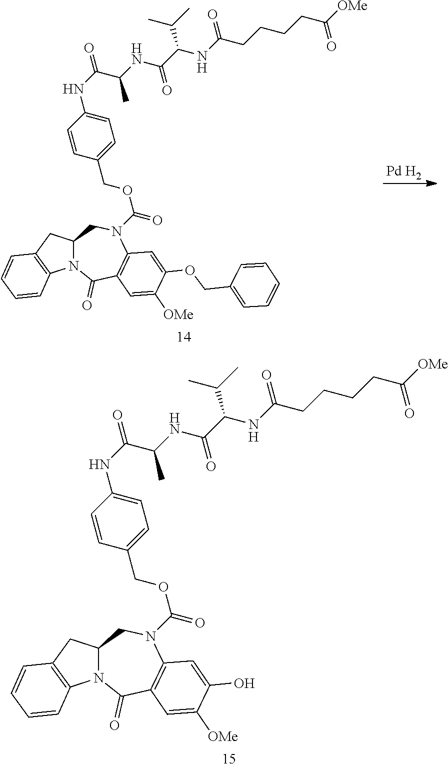 [SMILES: COC(=O)CCCCC(=O)N[C@H](C(=O)N[C@@H](C)C(=O)Nc1ccc(COC(=O)N2C[C@@H]3Cc4ccccc4N3C(=O)c3cc(OC)c(OCc4ccccc4)cc32)cc1)C(C)C ; "Pd H2" ; COC(=O)CCCCC(=O)N[C@H](C(=O)N[C@@H](C)C(=O)Nc1ccc(COC(=O)N2C[C@@H]3Cc4ccccc4N3C(=O)c3cc(OC)c(O)cc32)cc1)C(C)C]

To a solution of compound 14 (0.38 g, 0.448 mmol) in anhydrous methanol (6.40 ml) degassed with Argon, was added palladium on carbon 10% (0.048 g). The solution was degassed again and then stirred under an atmosphere of hydrogen at room temperature and monitored by UPLC until complete. After 1 hr and 30 minutes the reaction mixture was filtered through celite, rinsing with dichloromethane/methanol. The crude solid was purified by silica gel chromatography in dichloromethane/methanol and the pure fractions collected to give compound 15 (263 mg, y=77%). MS (m/z): 758.6 (M+1)⁺. UPLC=1.52 min (2.5 min method).

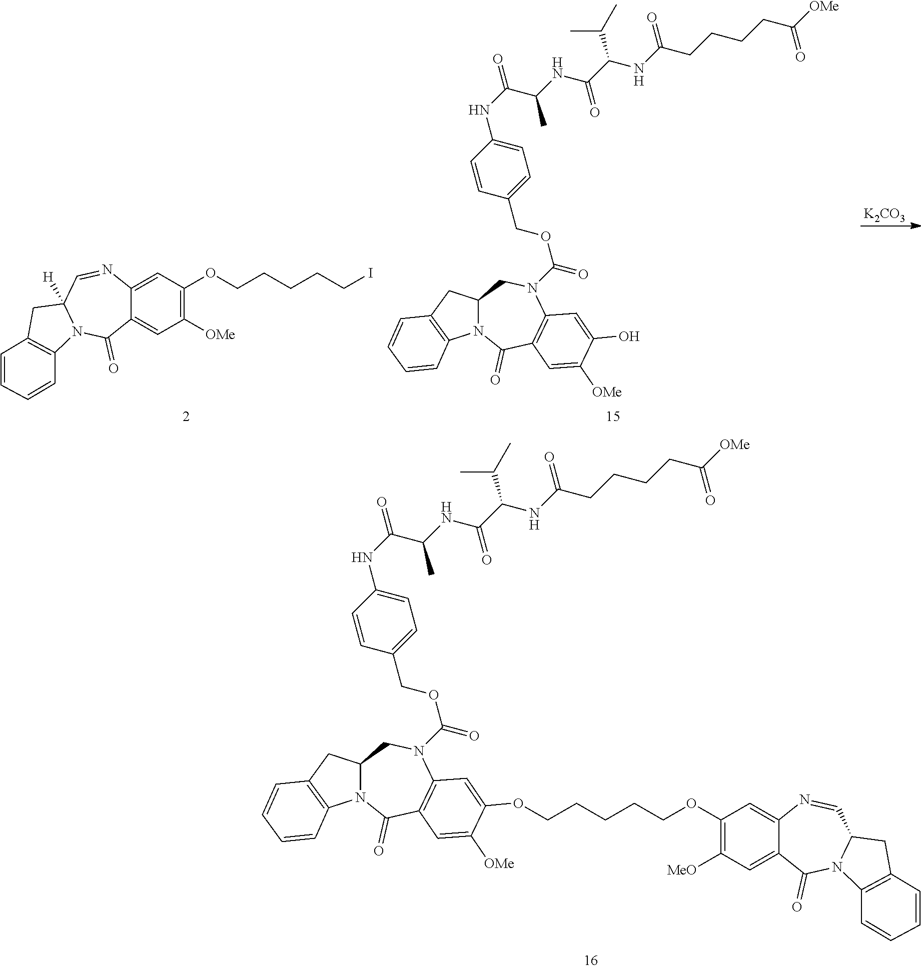 [SMILES: COc1cc2c(cc1OCCCCCI)N=C[C@@H]1Cc3ccccc3N1C2=O ; COC(=O)CCCCC(=O)N[C@H](C(=O)N[C@@H](C)C(=O)Nc1ccc(COC(=O)N2C[C@@H]3Cc4ccccc4N3C(=O)c3cc(OC)c(O)cc32)cc1)C(C)C ; "K2CO3" ; COC(=O)CCCCC(=O)N[C@H](C(=O)N[C@@H](C)C(=O)Nc1ccc(COC(=O)N2C[C@@H]3Cc4ccccc4N3C(=O)c3cc(OC)c(OCCCCCOc4cc5c(cc4OC)C(=O)N4c6ccccc6C[C@H]4C=N5)cc32)cc1)C(C)C]

Compound 15 (414 mg, 0.0546 mmol) and compound 2 (321 mg, 0.656 mmol) were dissolved in anhydrous dimethylacetamide (5463 μl). Potassium carbonate (151 mg, 1.093 mmol) was added and the reaction stirred overnight at room temperature under nitrogen. The reaction was precipitated with water, stirred for five minutes and filtered. The resulting solid was dissolved in 20% methanol/dichloromethane, transferred to a separatory funnel, washed with water, dried over anhydrous magnesium sulfate and concentrated in vacuo and purified via silica gel chromatography in dichloromethane/methanol. Evaporation of the pure fractions gave compound 16 (329 mg, y=54%). MS (m/z): 1121.3 (M+1)⁺. UPLC=1.88 min (2.5 min method).

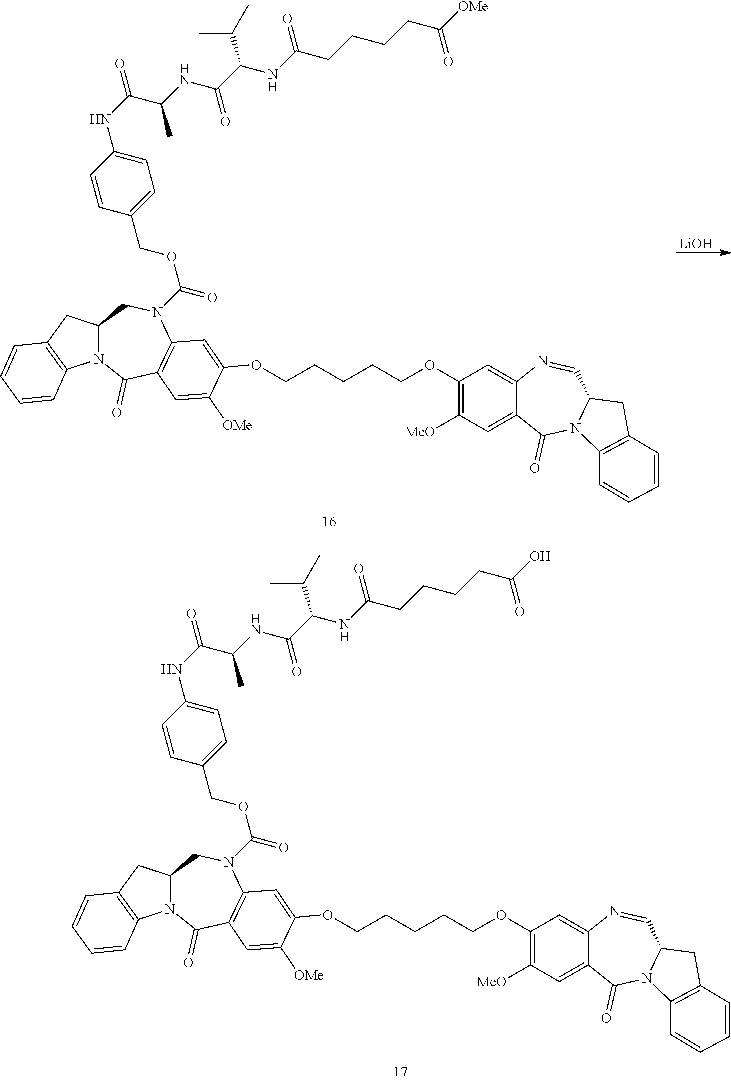 [SMILES: COC(=O)CCCCC(=O)N[C@H](C(=O)N[C@@H](C)C(=O)Nc1ccc(COC(=O)N2C[C@@H]3Cc4ccccc4N3C(=O)c3cc(OC)c(OCCCCCOc4cc5c(cc4OC)C(=O)N4c6ccccc6C[C@H]4C=N5)cc32)cc1)C(C)C ; "LiOH" ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)O)C(C)C)cc1)N=C[C@@H]1Cc3ccccc3N1C2=O]

Compound 16 (0.329 g, 0.294 mmol) was dissolved in anhydrous Tetrahydrofuran (11.01 ml) and deionized water (3.67 ml). Lithium hydroxide (0.021 g, 0.881 mmol) was added and the reaction monitored by UPLC until complete conversion to the desired product. After stirring for 1 hour and 30 minutes at room temperature it was diluted with 30% methanol/dichloromethane and deionized water, then slowly acidified with 0.5 M HCl to pH˜3. The acidified aqueous layer was extracted twice with 30% methanol/dichloromethane. The combined organics were washed with water to pH=5, dried with magnesium sulfate, filtered over celite, concentrated and co-evaporated with dichloromethane to give compound 17 as a yellow solid that was used without further purification. (290 mg, y=89%). MS (m/z): 1107.2 (M+1)⁺. UPLC=1.78 min (2.5 min method).

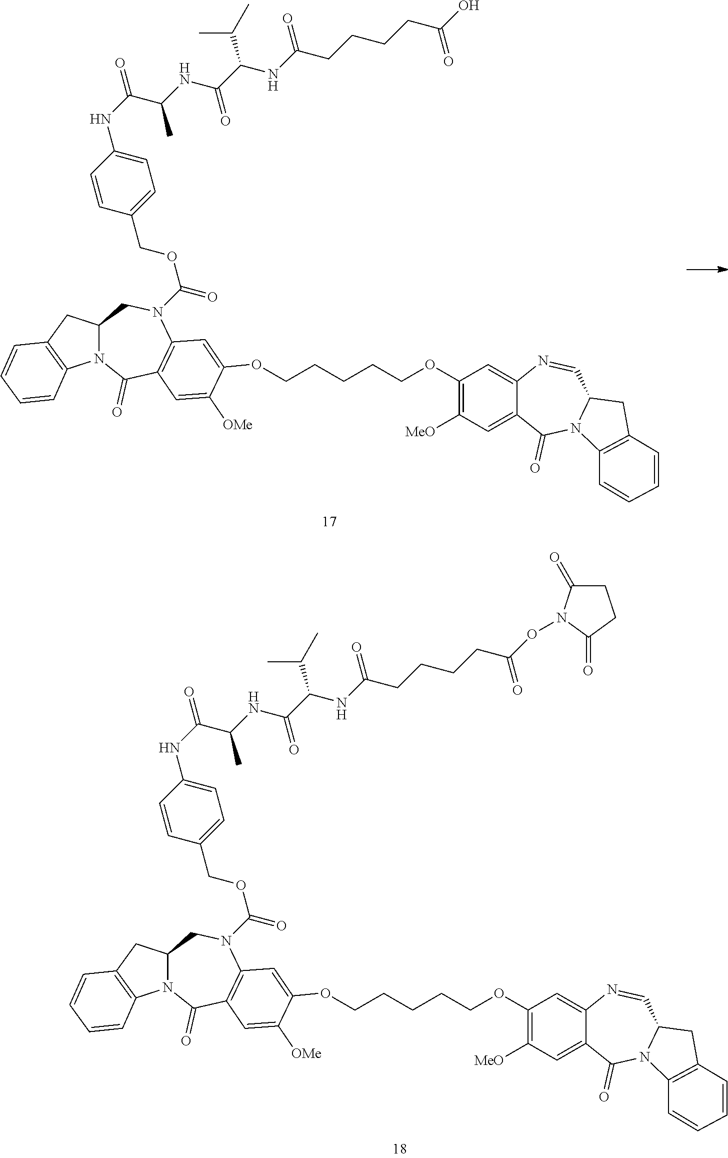 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)O)C(C)C)cc1)N=C[C@@H]1Cc3ccccc3N1C2=O ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)ON3C(=O)CCC3=O)C(C)C)cc1)N=C[C@@H]1Cc3ccccc3N1C2=O]

Compound 17 (0.29 g, 0.262 mmol) was suspended in anhydrous dichloromethane (6.55 ml). N-hydroxy succinimide (0.091 g, 0.786 mmol) and EDC.HCl (0.251 g, 1.311 mmol) were added and the starting material became soluble. The reaction was stirred at room temperature for 1 hour and was complete. The reaction was diluted with dichloromethane and washed with water. The organic was dried over magnesium sulfate, filtered and stripped to give 310 mgs crude material that was purified by RP-HPLC (C18 Kromasil, deionized water/acetonitrile). The fractions containing desired material were frozen and lyophilized to give pure final compound 18 (140 mgs, y=56%* based on recovery of amount purified). MS (m/z): 1204.4 (M+1)⁺. UPLC=1.86 min (2.5 min method).

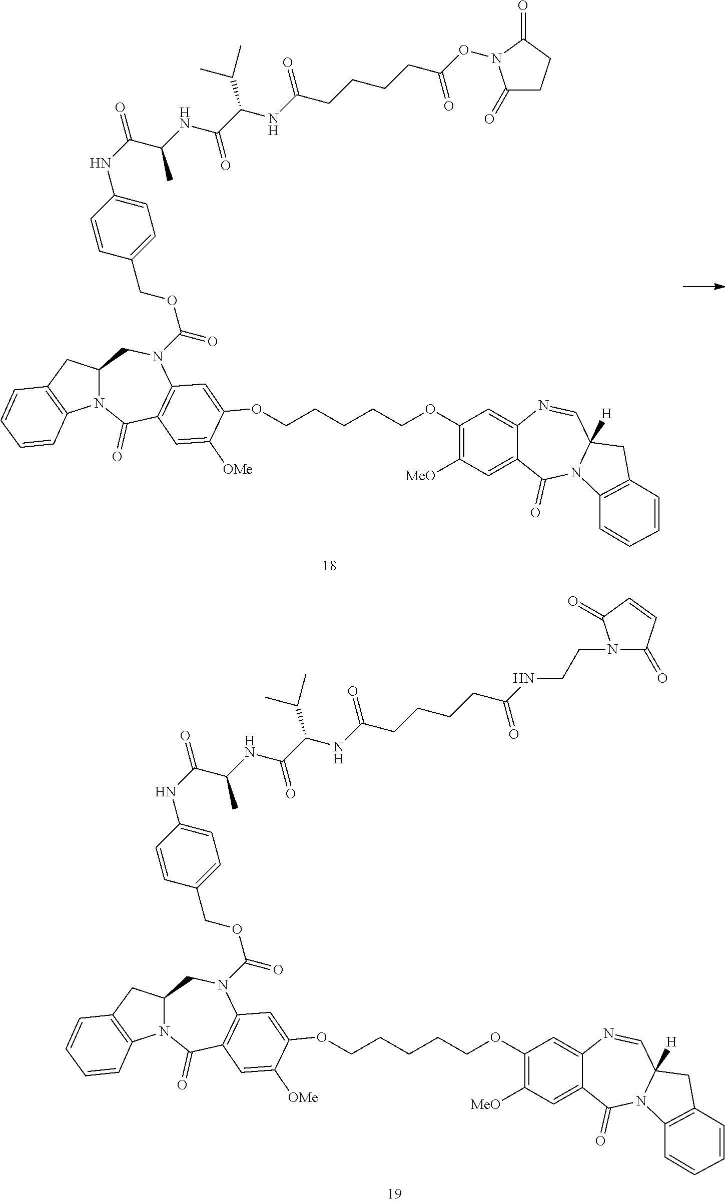 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)ON3C(=O)CCC3=O)C(C)C)cc1)N=C[C@@H]1Cc3ccccc3N1C2=O ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)NCCN3C(=O)C=CC3=O)C(C)C)cc1)N=C[C@@H]1Cc3ccccc3N1C2=O]

Compound 18 (96.6 mg, 0.080 mmol) was dissolved in anhydrous dichloromethane (3211 μl). 1-(2-aminoethyl)-1H-pyrrole-2,5-dione hydrochloride (18.29 mg, 0.096 mmol) was added followed by anhydrous DIPEA (28.0 μl, 0.161 mmol). The reaction was monitored by UPLC and after 1 hour and 30 minutes was concentrated to dryness. The crude solid was redissolved in acetonitrile/deionized water and a few drops formic acid and purified by RP-HPLC (C18 Kromasil, deionized water/acetonitrile). The fractions containing desired material were frozen and lyophilized to give pure final compound 19 (54 mgs, y=55%). MS (m/z): 1229.4 (M+1)⁺. UPLC=1.78 min (2.5 min method).

### Example 2. Synthesis of Compound 23

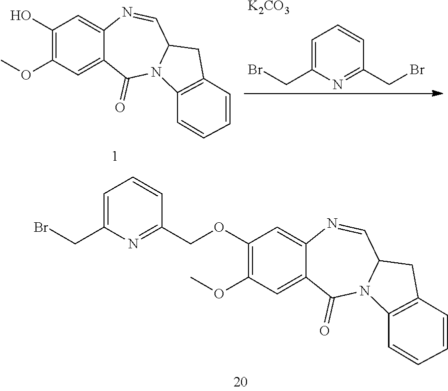

Compound 1 (0.173 g, 0.588 mmol) was dissolved in dimethylformamide (3.92 ml) and 2,6-bis(bromomethyl)pyridine (1.090 g, 4.11 mmol) and potassium carbonate (0.203 g, 1.470 mmol) were added. The reaction stirred for 4 hours 30 min at which compound 1 was consumed. The reaction was diluted with ethyl acetate and washed with saturated ammonium chloride, water, and brine. The crude material was dissolved in dichloromethane and purified by silica gel chromatography (dichloromethane/ethyl acetate). The pure fractions were combined to give compound 20 (193 mgs, y=68%). MS (m/z): 478.3 (M+1)⁺. UPLC=1.6 min (2.5 min method).

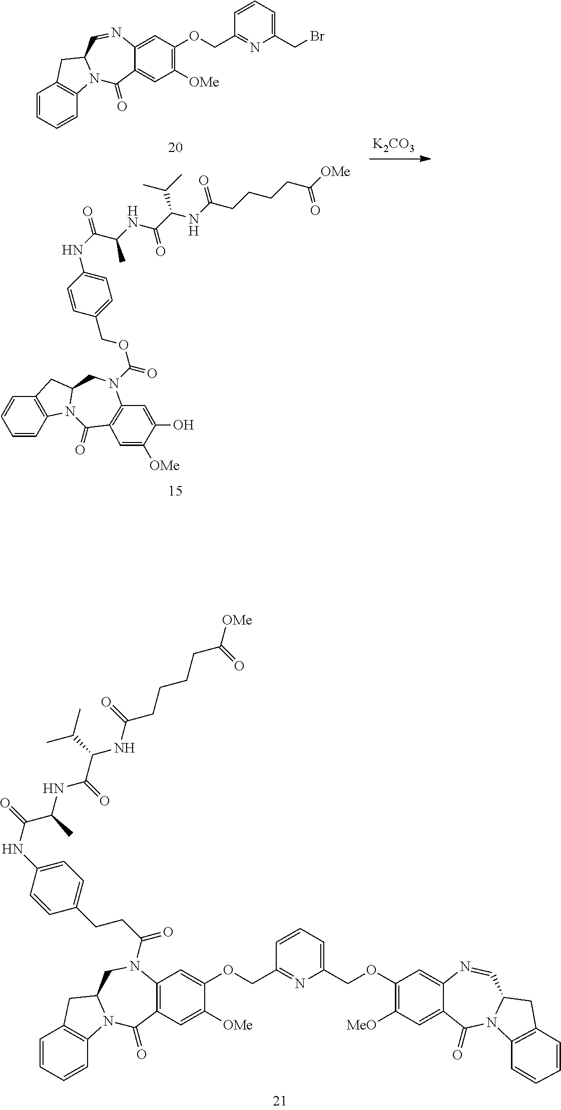 [SMILES: COc1cc2c(cc1OCc1cccc(CBr)n1)N=C[C@@H]1Cc3ccccc3N1C2=O ; "K2CO3" ; COC(=O)CCCCC(=O)N[C@H](C(=O)N[C@@H](C)C(=O)Nc1ccc(COC(=O)N2C[C@@H]3Cc4ccccc4N3C(=O)c3cc(OC)c(O)cc32)cc1)C(C)C ; COC(=O)CCCCC(=O)N[C@H](C(=O)N[C@@H](C)C(=O)Nc1ccc(CCC(=O)N2C[C@@H]3Cc4ccccc4N3C(=O)c3cc(OC)c(OCc4cccc(COc5cc6c(cc5OC)C(=O)N5c7ccccc7C[C@H]5C=N6)n4)cc32)cc1)C(C)C]

Compound 15 (86 mg, 0.113 mmol) and compound 20 (65.1 mg, 0.136 mmol) were dissolved in anhydrous dimethylacetamide (1135 μl). Potassium carbonate (31.4 mg, 0.227 mmol) was added and the reaction was stirred overnight at room temperature. The reaction was precipitated with water, stirred for 5 minutes, and filtered. The resulting solid was dissolved in 20% methanol/dichloromethane, transferred to a separatory funnel, washed with water, dried over anhydrous magnesium sulfate and concentrated. The crude material was purified via silica gel column chromatography (dichloromethane/methanol) to give compound 21 (96 mg, y=73%). MS (m/z): 1156.2 (M+1)⁺. UPLC=1.84 (2.5 min method).

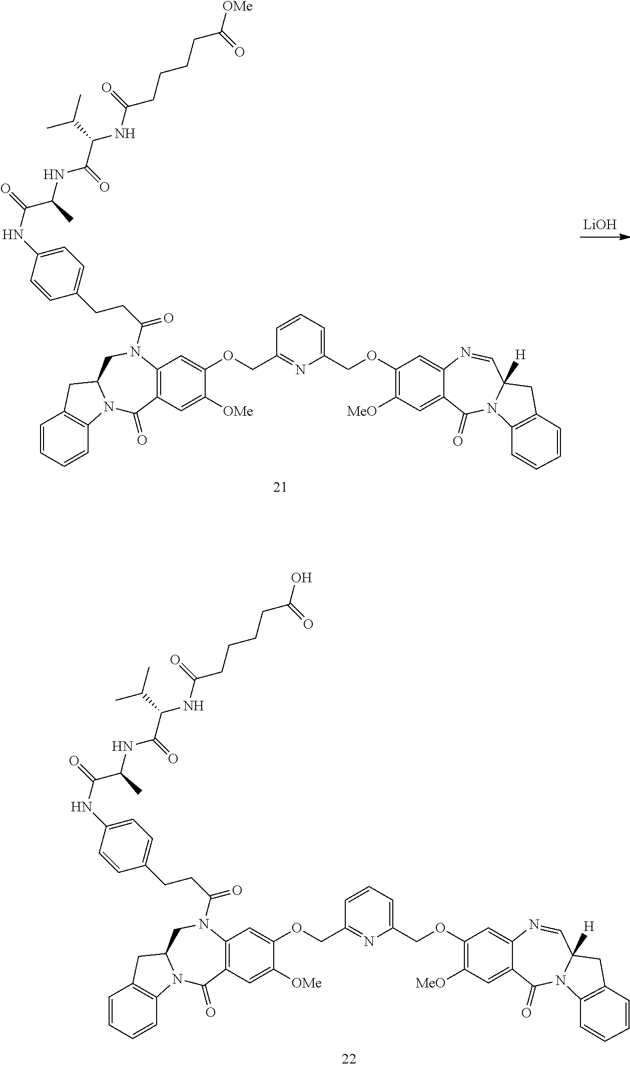 [SMILES: COC(=O)CCCCC(=O)N[C@H](C(=O)N[C@@H](C)C(=O)Nc1ccc(CCC(=O)N2C[C@@H]3Cc4ccccc4N3C(=O)c3cc(OC)c(OCc4cccc(COc5cc6c(cc5OC)C(=O)N5c7ccccc7C[C@H]5C=N6)n4)cc32)cc1)C(C)C ; "LiOH" ; COc1cc2c(cc1OCc1cccc(COc3cc4c(cc3OC)C(=O)N3c5ccccc5C[C@H]3CN4C(=O)CCc3ccc(NC(=O)[C@H](C)NC(=O)[C@@H](NC(=O)CCCCC(=O)O)C(C)C)cc3)n1)N=C[C@@H]1Cc3ccccc3N1C2=O]

Compound 22 was prepared similarly as Compound 17. The crude material was used without further purification to give (94 mgs, y=99%). MS (m/z): 1142.2 (M+1)⁺. UPLC=1.76 (2.5 min method).

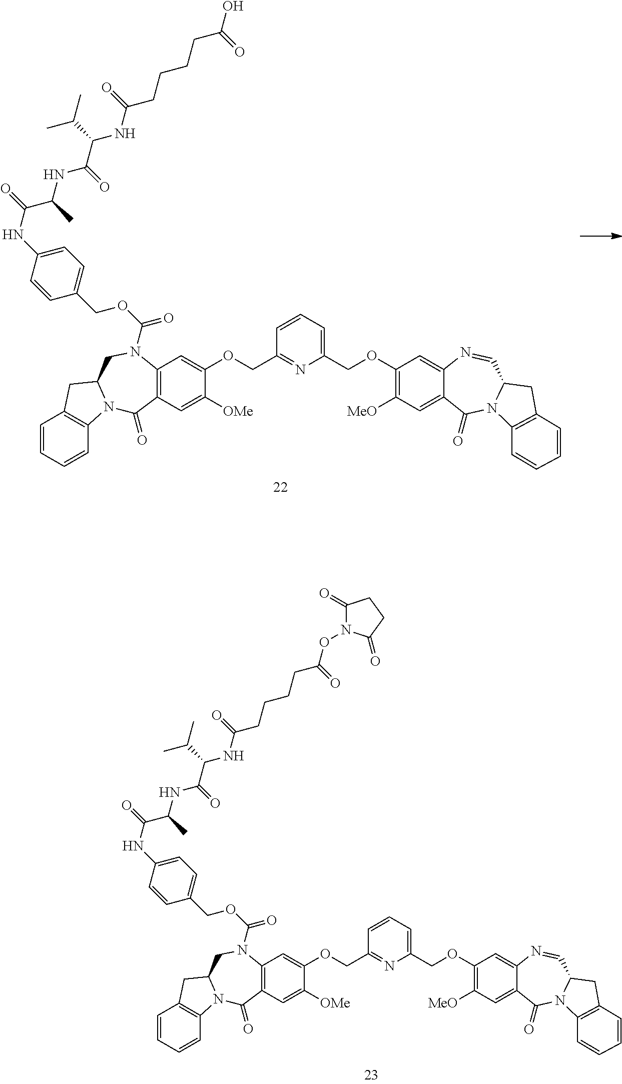 [SMILES: COc1cc2c(cc1OCc1cccc(COc3cc4c(cc3OC)C(=O)N3c5ccccc5C[C@H]3CN4C(=O)OCc3ccc(NC(=O)[C@H](C)NC(=O)[C@@H](NC(=O)CCCCC(=O)O)C(C)C)cc3)n1)N=C[C@@H]1Cc3ccccc3N1C2=O ; COc1cc2c(cc1OCc1cccc(COc3cc4c(cc3OC)C(=O)N3c5ccccc5C[C@H]3CN4C(=O)OCc3ccc(NC(=O)[C@H](C)NC(=O)[C@@H](NC(=O)CCCCC(=O)ON4C(=O)CCC4=O)C(C)C)cc3)n1)N=C[C@@H]1Cc3ccccc3N1C2=O]

Compound 23 was prepared similarly as Compound 18. The crude material was purified via RPHPLC (C18 column, acetonitrile/water) to give the final compound 23 (41 mg, y=40%). MS (m/z): 1239.3 (M+1)⁺. UPLC=1.82 min (2.5 min method).

### Example 3. Synthesis of Compounds 25 and 26

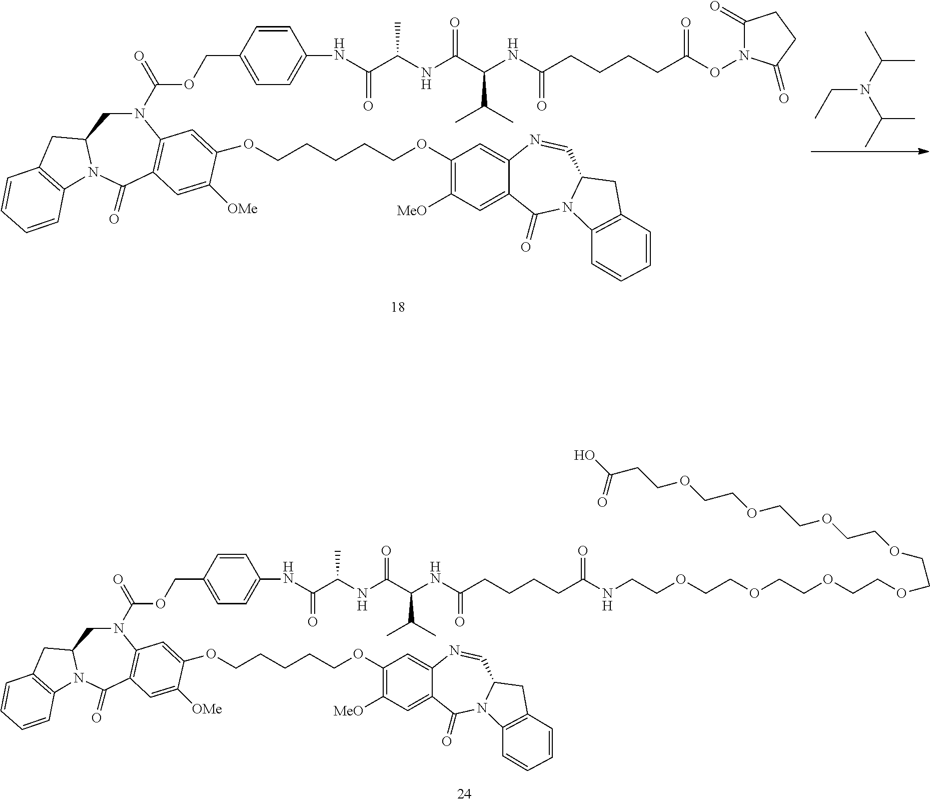

Compound 18 (49 mg, 0.041 mmol) was dissolved in anhydrous dichloromethane (1629 μl). 1-amino-3,6,9,12,15,18,21,24-octaoxaheptacosan-27-oic acid (19.78 mg, 0.045 mmol) and DIPEA (10.64 μl, 0.061 mmol) were added at room temperature. The reaction stirred for 1 hour and 30 minutes and was then concentrated to dryness. The crude residue was purified by silica gel chromatography in dichloromethane/methanol. The pure fractions were collected and coevaporated with dichloromethane to give compound 24 as a fluffy white solid (39 mg, y=63%). UPLC=1.60 min (2.5 min method).

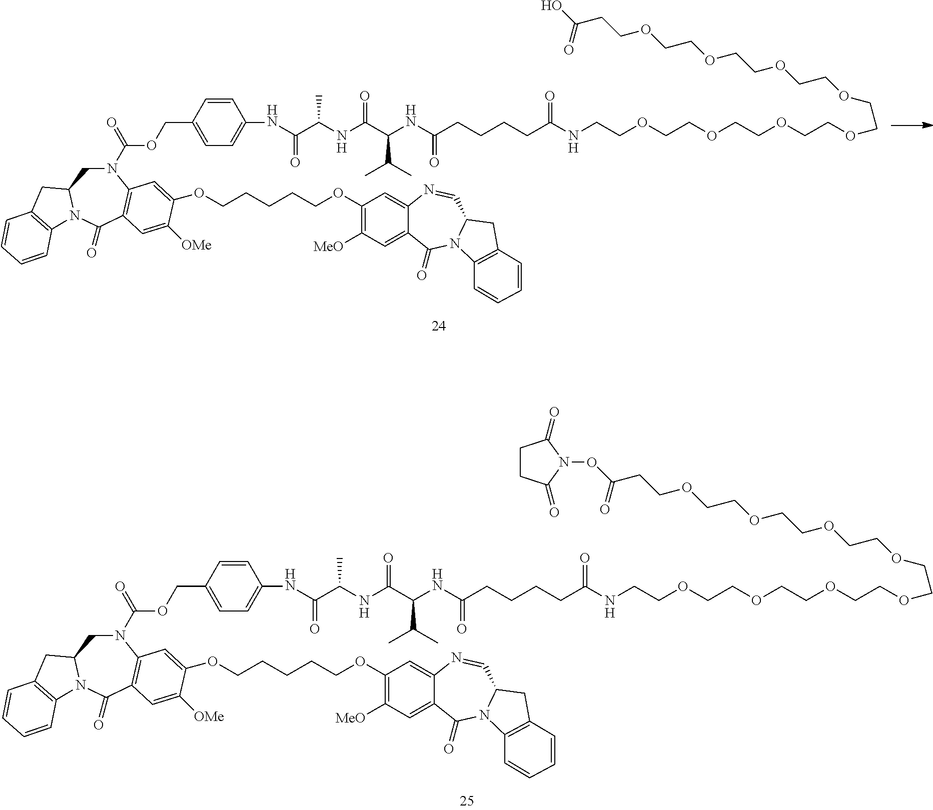 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)NCCOCCOCCOCCOCCOCCOCCOCCOCCC(=O)O)C(C)C)cc1)N=C[C@@H]1Cc3ccccc3N1C2=O ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)NCCOCCOCCOCCOCCOCCOCCOCCOCCC(=O)ON3C(=O)CCC3=O)C(C)C)cc1)N=C[C@@H]1Cc3ccccc3N1C2=O]

Compound 25 was prepared similarly as Compound 18. The crude material was used directly in the next reaction or purified via RPHPLC (C18 column, acetonitrile/water) to give the final pure compound 25 (10 mg, y=49%). MS (m/z): 1627.2 (M+1)⁺. UPLC=1.66 min (2.5 min method).

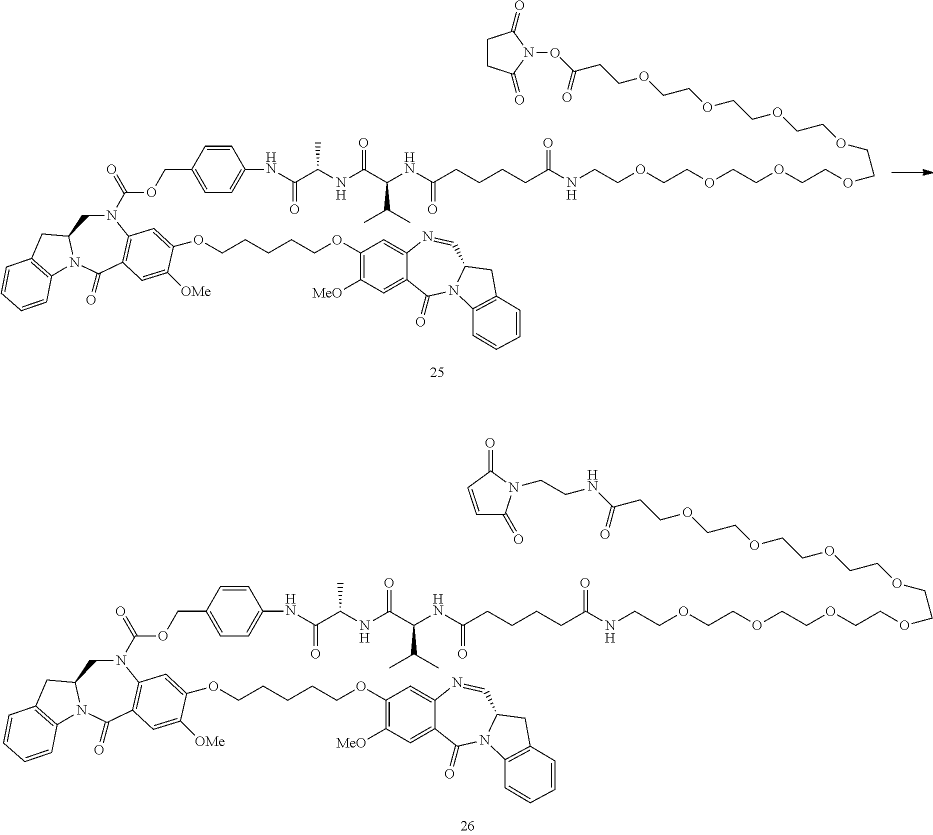 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)NCCOCCOCCOCCOCCOCCOCCOCCOCCC(=O)ON3C(=O)CCC3=O)C(C)C)cc1)N=C[C@@H]1Cc3ccccc3N1C2=O ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)NCCOCCOCCOCCOCCOCCOCCOCCOCCC(=O)NCCN3C(=O)C=CC3=O)C(C)C)cc1)N=C[C@@H]1Cc3ccccc3N1C2=O]

Compound 26 was prepared similarly as Compound 19. The crude material was purified via RPHPLC (C18 column, acetonitrile/water) to give the final pure compound 26 (12 mg, y=48%). MS (m/z): 1652.2 (M+1)⁺. UPLC=1.60 min (2.5 min method).

### Example 4. Synthesis of Compound 36 and Compound 37

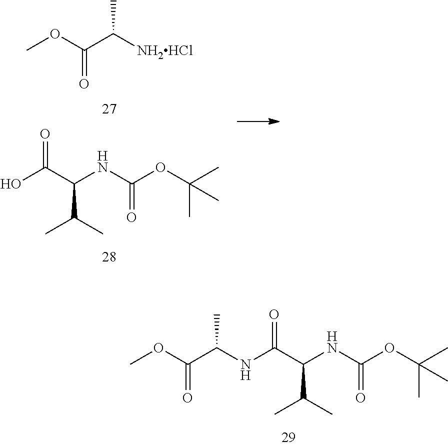

(tert-butoxycarbonyl)-L-valine (10.58 g, 48.7 mmol) was dissolved in dichloromethane (97 ml). CDI (9.48 g, 58.4 mmol) was added in portions at room temperature. The mixture stirred at room temperature under Argon for 2.5 hours and the reaction had turned yellow. Methyl L-alaninate hydrochloride (7.00 g, 50.2 mmol) was added and the mixture became pale yellow. It continued stirring under argon overnight. The mixture was diluted with dichloromethane, washed with 1 M aqueous HCl (×2), saturated aqueous sodium bicarbonate, and brine. The organic was dried over anhydrous magnesium sulfate, filtered and concentrated to give compound 29 as a white solid that was used crude without further purification (12.85 g, y=87%).

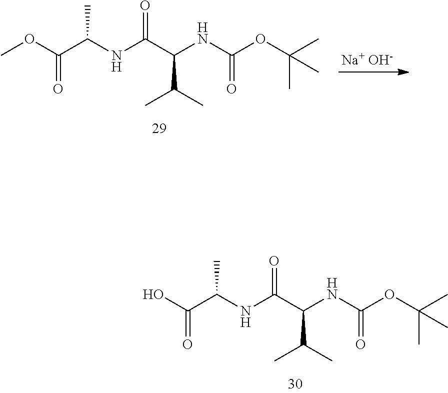 [SMILES: "Na+ OH-" ; COC(=O)[C@H](C)NC(=O)[C@@H](NC(=O)OC(C)(C)C)C(C)C ; CC(C)[C@H](NC(=O)OC(C)(C)C)C(=O)N[C@@H](C)C(=O)O]

Compound 29 (12.85 g, 42.5 mmol) was dissolved in methanol (42.5 ml) and cooled to 0C. 1 M Sodium Hydroxide (47.8 ml, 47.8 mmol) was added to reaction and the reaction was monitored by TLC with bromocresol green stain. After 2.5 hours water was added to clear the hazy solution. The solution was then acidified to pH-3-4 with 1M HCl while in ice bath causing precipitate to form. It was removed from the ice bath and extracted with ethyl acetate three times, washed with brine, dried over sodium sulfate, filtered and stripped to give a white sticky solid. The crude material was placed on the high vacuum and used without further purification to give compound 30 (12.05 g, y=98%).

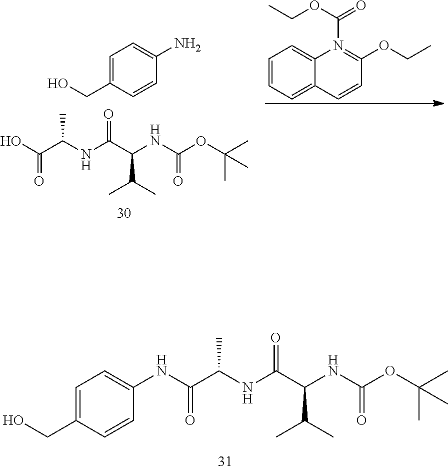

Compound 30 (12.05 g, 41.8 mmol) was suspended in anhydrous dichloromethane (139 ml) and methanol (69.7 ml). EEDQ (18.60 g, 75 mmol) and then (4-aminophenyl)methanol (6.18 g, 50.1 mmol) were added and the reaction was stirred overnight under Argon at room temperature. The reaction mixture was concentrated and coevaporated with dichloromethane. The solid was triturated with ethyl acetate and then purified by silica gel chromatography (dichloromethane/methanol) to give compound 31 (5.45 g, y=33%).

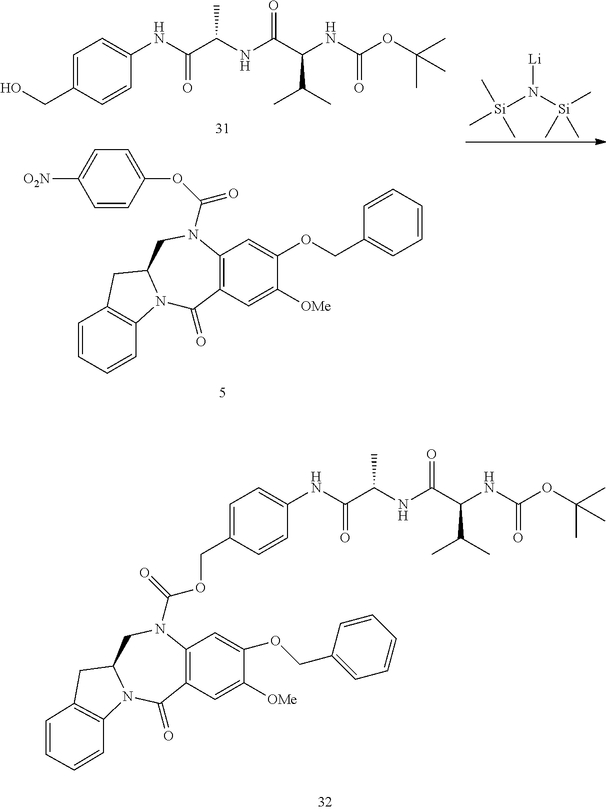

Compound 31 and compound 5 were reacted similarly to the formation of compound 14. The material was purified by silica gel chromatography (dichloromethane/methanol) to give compound 32 (507 mg, y=54%). MS (m/z): 806.5 (M+1)⁺ and 804.5 (M−1)⁻. UPLC=1.86 min (2.5 min method).

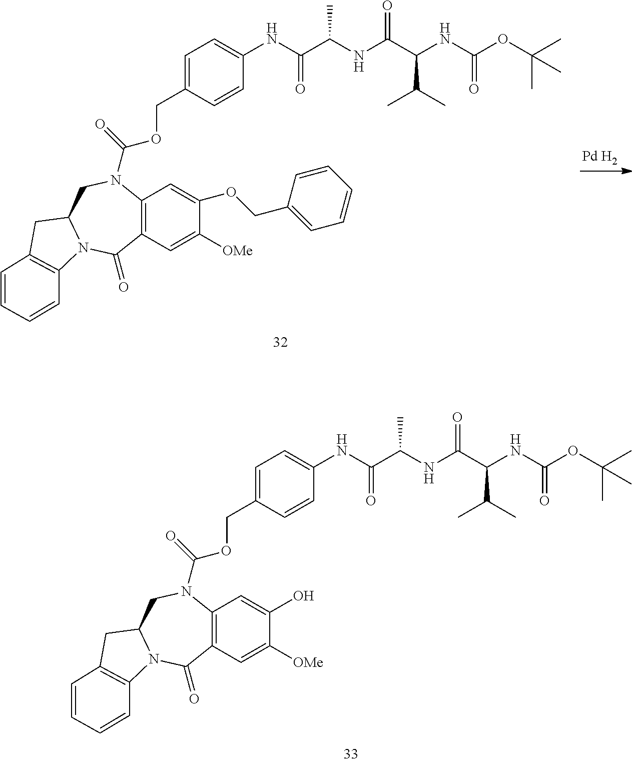 [SMILES: COc1cc2c(cc1OCc1ccccc1)N(C(=O)OCc1ccc(NC(=O)[C@H](C)NC(=O)[C@@H](NC(=O)OC(C)(C)C)C(C)C)cc1)C[C@@H]1Cc3ccccc3N1C2=O ; "Pd H2" ; COc1cc2c(cc1O)N(C(=O)OCc1ccc(NC(=O)[C@H](C)NC(=O)[C@@H](NC(=O)OC(C)(C)C)C(C)C)cc1)C[C@@H]1Cc3ccccc3N1C2=O]

Compound 33 was prepared similarly as Compound 15. The crude material was purified by silica gel chromatography to give (213 mg, y=47%). MS (m/z): 716.6 (M+1)⁺ and 714.5 (M−1)⁻. UPLC=1.61 min (2.5 min method).

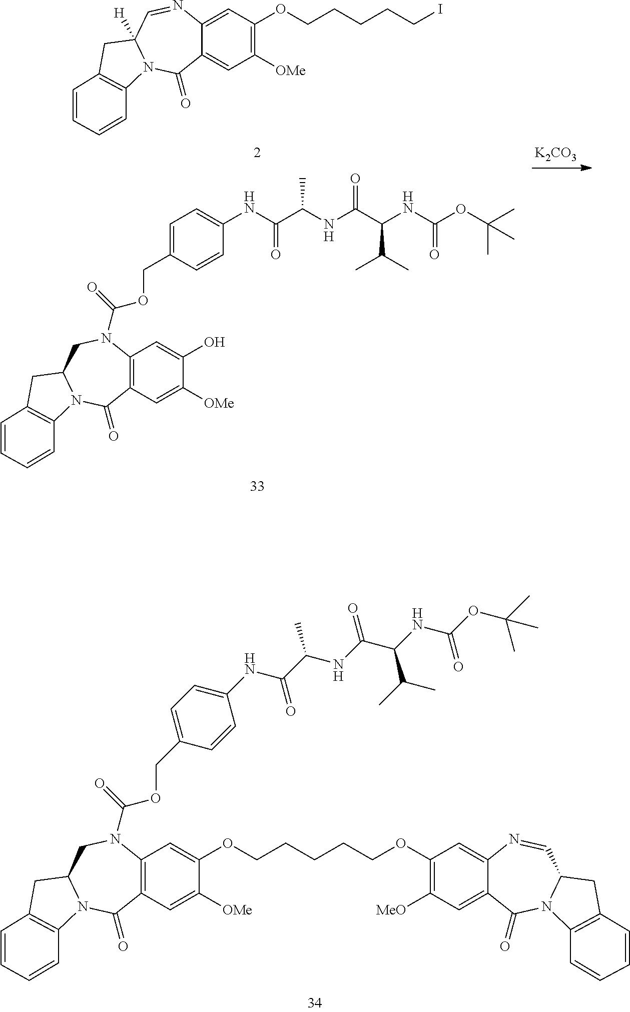 [SMILES: COc1cc2c(cc1OCCCCCI)N=C[C@@H]1Cc3ccccc3N1C2=O ; "K2CO3" ; COc1cc2c(cc1O)N(C(=O)OCc1ccc(NC(=O)[C@H](C)NC(=O)[C@@H](NC(=O)OC(C)(C)C)C(C)C)cc1)C[C@@H]1Cc3ccccc3N1C2=O ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)OC(C)(C)C)C(C)C)cc1)N=C[C@@H]1Cc3ccccc3N1C2=O]

Compound 2 and compound 33 were reacted similar as in the preparation of compound 16. The crude material was purified by silica gel chromatography (dichloromethane/methanol) to give compound 34 (140 mg, y=94%). MS (m/z): 1078.7 (M+1)⁺. UPLC=1.84 min (2.5 min method).

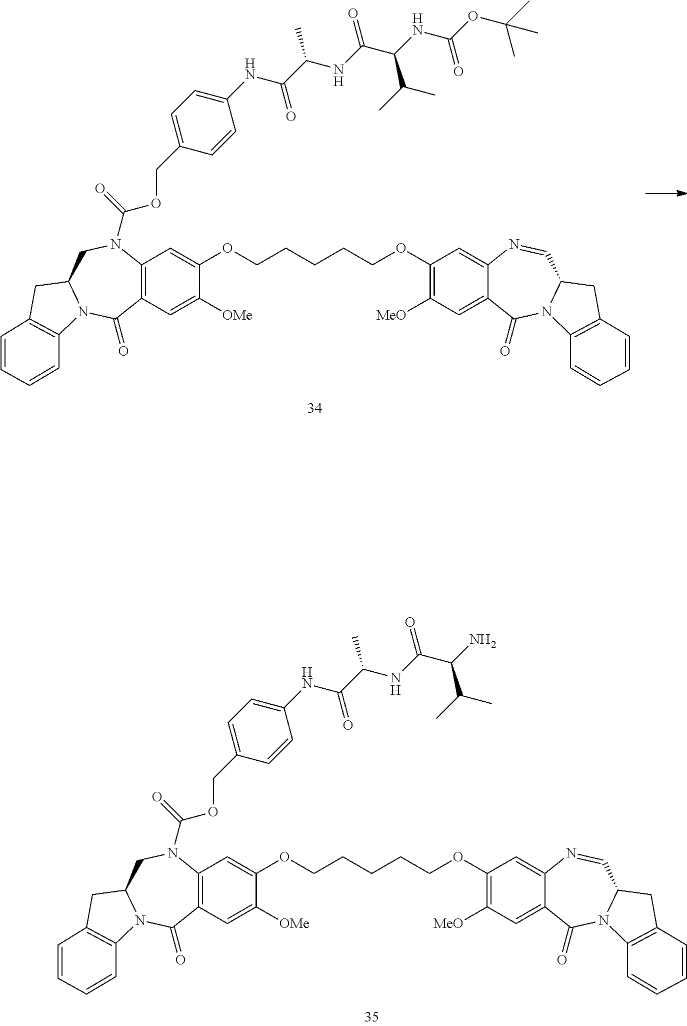 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)OC(C)(C)C)C(C)C)cc1)N=C[C@@H]1Cc3ccccc3N1C2=O ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](N)C(C)C)cc1)N=C[C@@H]1Cc3ccccc3N1C2=O]

Compound 34 (70 mg, 0.065 mmol) was dissolved in anhydrous dichloromethane (600 μl) and cooled to OC in an ice bath. A freshly mixed solution of anhydrous dichloromethane (600 μl) and TFA (601 μl) was added and the solution turned bright yellow. The reaction was monitored by UPLC and stirred under argon at OC for 50 minutes until complete consumption of the starting material. It was diluted with dichloromethane and poured into an ice/saturated sodium bicarbonate solution. The separated organic was washed with brine, dried over magnesium sulfate, filtered and stripped to give compound 35 (55 mg, y=87%) as a pale yellow solid that was used directly without further purification. MS (m/z): 978.7 (M+1)⁺. UPLC=1.44 min (2.5 min method).

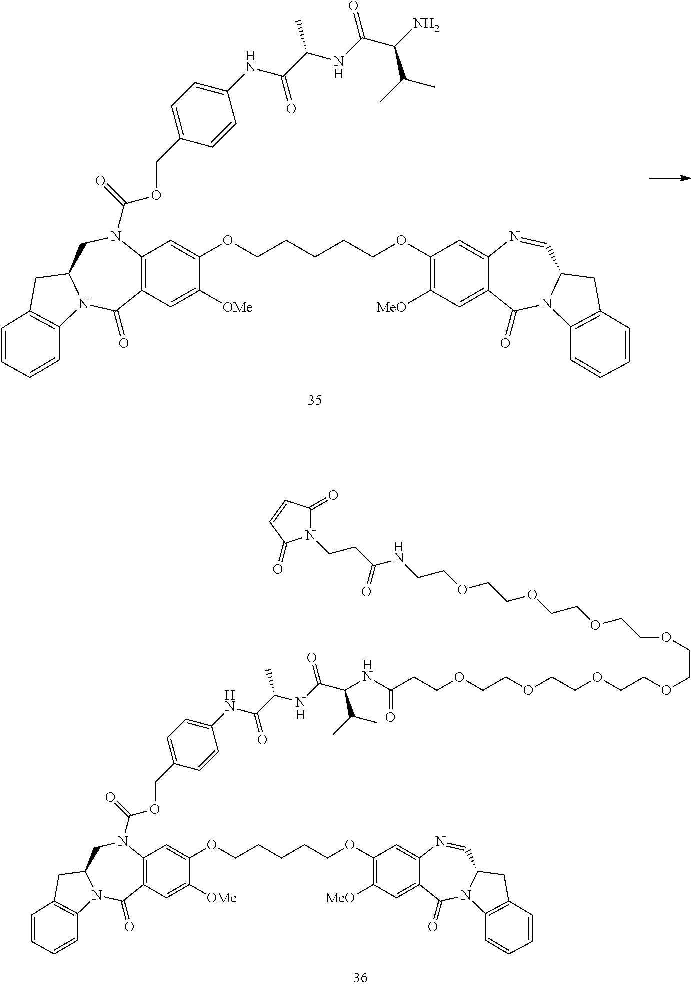 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](N)C(C)C)cc1)N=C[C@@H]1Cc3ccccc3N1C2=O ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)cc1)N=C[C@@H]1Cc3ccccc3N1C2=O]

Compound 35 (55 mg, 0.056 mmol) and 1-(2,5-dioxo-2,5-dihydro-1H-pyrrol-1-yl)-3-oxo-7,10,13,16,19,22,25,28-octaoxa-4-azahentriacontan-31-oic acid (33.3 mg, 0.056 mmol) were dissolved in anhydrous dichloromethane (3514 μl). EDC (10.78 mg, 0.056 mmol) was added and the reaction was degassed three times and stirred at room temperature under argon. It was monitored by UPLC and after 1 hour the reaction was diluted with dichloromethane and washed with water and brine. The organic was dried with magnesium sulfate, filtered and stripped to give 65 mg crude. The crude material was purified via RPHPLC (C18 column, acetonitrile/water) to give the final pure compound 36 (48 mg, y=55%). MS (m/z): 1553.7 (M+1)⁺. UPLC=1.63 min (2.5 min method).

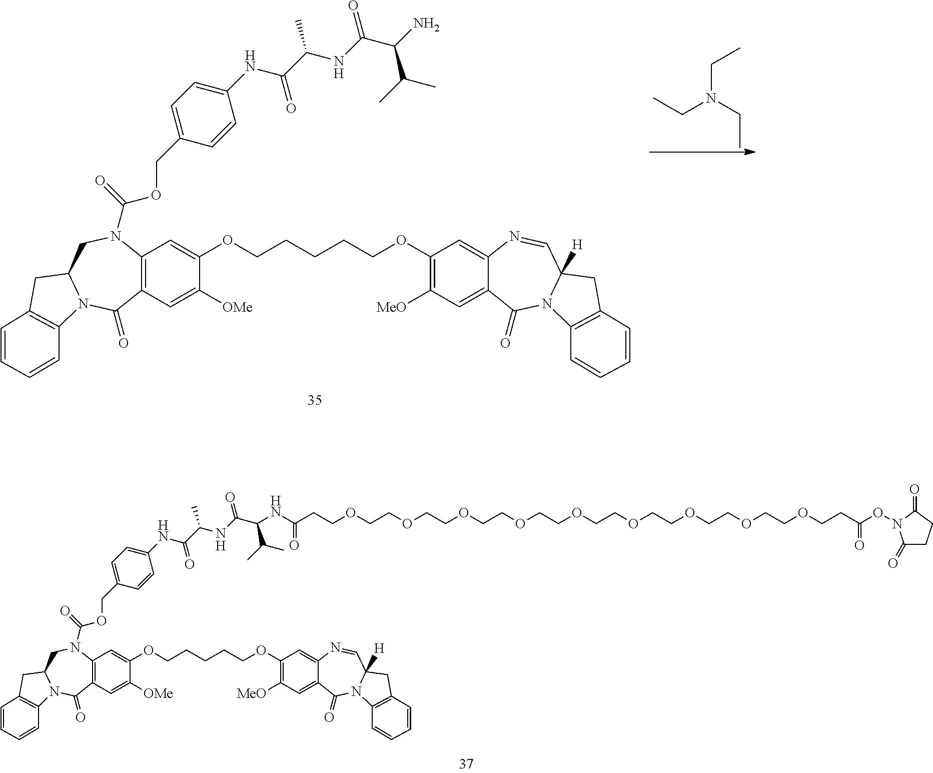

Bis(2,5-dioxopyrrolidin-1-yl) 4,7,10,13,16,19,22,25,28-nonaoxahentriacontanedioate (34.8 mg, 0.049 mmol) was dissolved in anhydrous dimethylformamide (981 μl) and added directly to compound 35 (48 EDC 0.7 m, 0 mol). Added Triethylamine (10.26 μl, 0.074 mmol) and then degassed the reaction and stirred at room temperature under argon. An additional 40 μl of triethylamine was added and the reaction was heated to 35C for 2.5 hours and then stirred at room temperature overnight. The reaction was diluted with water and dichloromethane. The aqueous contained all desired product. Added acetonitrile to the separated aqueous, froze and lyophilized. Dissolved the crude lyophilized material in dimethylacetamide and purified via RPHPLC (water/acetonitrile). Froze and lyophilized the pure fractions to give compound 37 (8 mg, y=10%). MS (m/z): 1572.4 (M+1)⁺. UPLC=1.70 min (2.5 min method).

### Example 5. Synthesis of Compound 46 and Compound 47

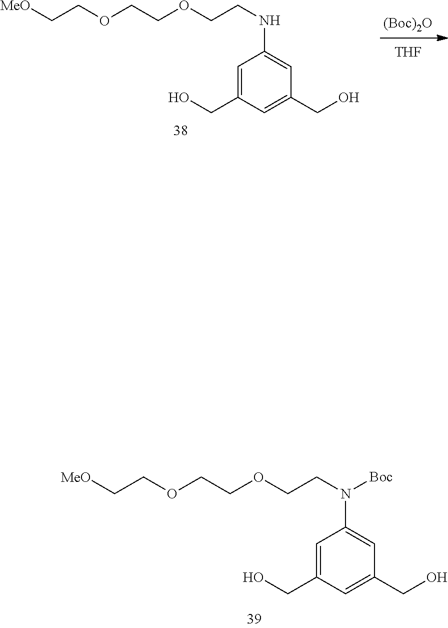 [SMILES: "(Boc)2O" ; COCCOCCOCCNc1cc(CO)cc(CO)c1 ; COCCOCCOCCN(C(=O)OC(C)(C)C)c1cc(CO)cc(CO)c1 ; "THF"]

To a stirred solution of the aniline 38 (339 mg, 1.1 mmol) in anhydrous tetrahydrofuran (4.0 mL) was added Boc anhydride (272 mg, 1.2 mmol). The mixture was continued to be stirred at room temperature for three days. The reaction mixture was concentrated under reduced pressure and the residue was purified by silica gel chromatography (dichloromethane/methanol) to give compound 39 (405 mg, y=90%) as colorless oil. ¹H NMR (400 Hz, CDCl₃): δ 7.00 (s, 2H), 6.97 (s, 1H), 4.38 (s, 4H), 4.12 (s, 2h), 3.64 (t, J=5.6 Hz, 2H), 3.48-3.44 (m, 8H), 3.40-3.38 (m, 2H), 3.21 (s, 3H), 1.31 (s, 9H); ³C NMR (400 Hz, CDCl₃): δ 154.65, 142.3, 142.1, 124.1, 122.7, 80.2, 71.6, 70.3, 70.1, 69.9, 68.5, 63.9, 58.65, 49.4, 28.1.

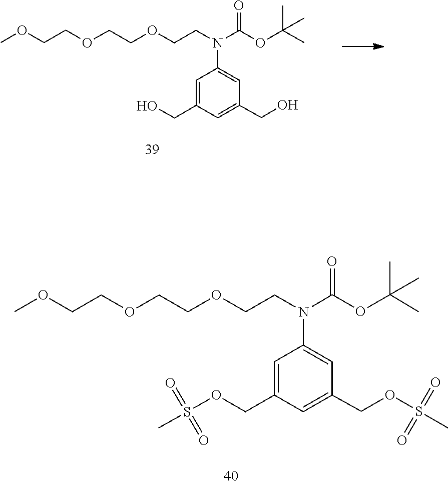

Compound 39 (3 g, 7.51 mmol) was dissolved in anhydrous dichloromethane (75 ml) and cooled to −5° C. in an acetone/dry ice bath. Triethylamine (5.23 ml, 37.5 mmol) was added followed by the addition of methanesulfonic anhydride (3.27 g, 18.77 mmol) and the resulting mixture was stirred at −5° C. for two hours. The reaction was diluted with cold ethyl acetate and washed with ice water twice. The filtrate was dried over anhydrous magnesium sulfate, filtered and concentrated in vacuo to give compound 40 (3.58 g, y=86% yield). The crude material was placed on high vacuum and then used directly without further purification. MS (m/z): 556.2 (M+1)⁺. UPLC=1.48 min (2.5 min method).

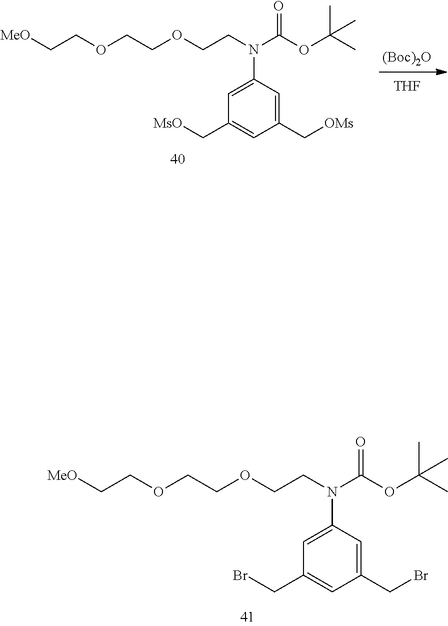 [SMILES: "(Boc)2O" ; COCCOCCOCCN(C(=O)OC(C)(C)C)c1cc(COS(C)(=O)=O)cc(COS(C)(=O)=O)c1 ; COCCOCCOCCN(C(=O)OC(C)(C)C)c1cc(CBr)cc(CBr)c1 ; "THF"]

Compound 40 (3.58 g, 6.44 mmol) was dissolved in dimethylformamide (32.2 ml). Sodium bromide (3.31 g, 32.2 mmol) was added and the reaction stirred at room temperature overnight to give one new peak by UPLC. Diluted the reaction mixture with water and extracted with ethyl acetate. Washed organic with water and dried over anhydrous magnesium sulfate, filtered and stripped to give compound 41 (3.01 g, y=89%) that was carried on without further purification. UPLC=1.79 min (2.5 min method).

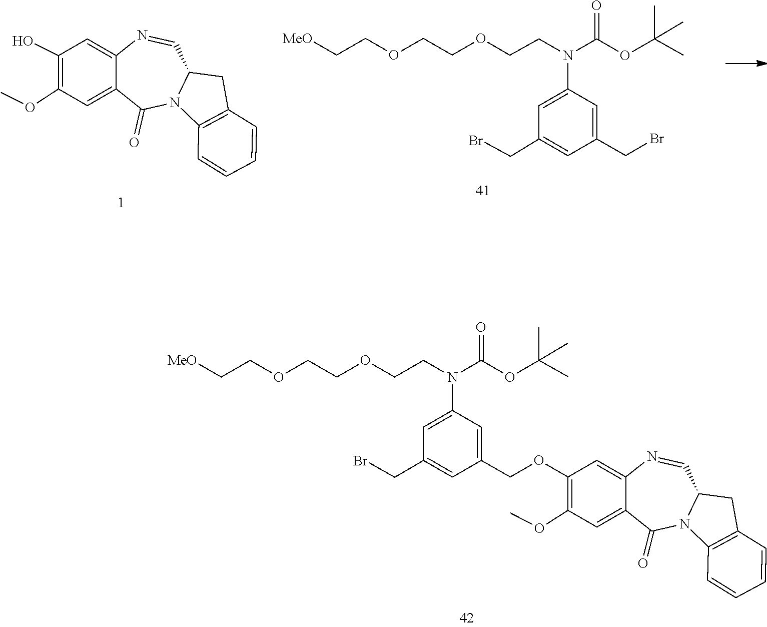 [SMILES: COc1cc2c(cc1O)N=C[C@@H]1Cc3ccccc3N1C2=O ; COCCOCCOCCN(C(=O)OC(C)(C)C)c1cc(CBr)cc(CBr)c1 ; COCCOCCOCCN(C(=O)OC(C)(C)C)c1cc(CBr)cc(COc2cc3c(cc2OC)C(=O)N2c4ccccc4C[C@H]2C=N3)c1]

Compound 1 (0.241 g, 0.819 mmol) and compound 41 (3.01 g, 5.73 mmol) were dissolved in dimethylformamide (5.46 ml). Potassium carbonate (0.283 g, 2.047 mmol) was added and the reaction stirred at room temperature for 2.5 hours until complete consumption of the compound 1. The reaction was diluted with ethyl acetate and washed with saturated ammonium chloride, water, and brine. The crude material was dissolved in dichloromethane and purified by silica gel chromatography (dichloromethane/ethyl acetate). The pure monocoupled material was collected to give compound 42 (322 mg, y=53%). MS (m/z): 738.3 (M+1)⁺. UPLC=1.80 min (2.5 min method).

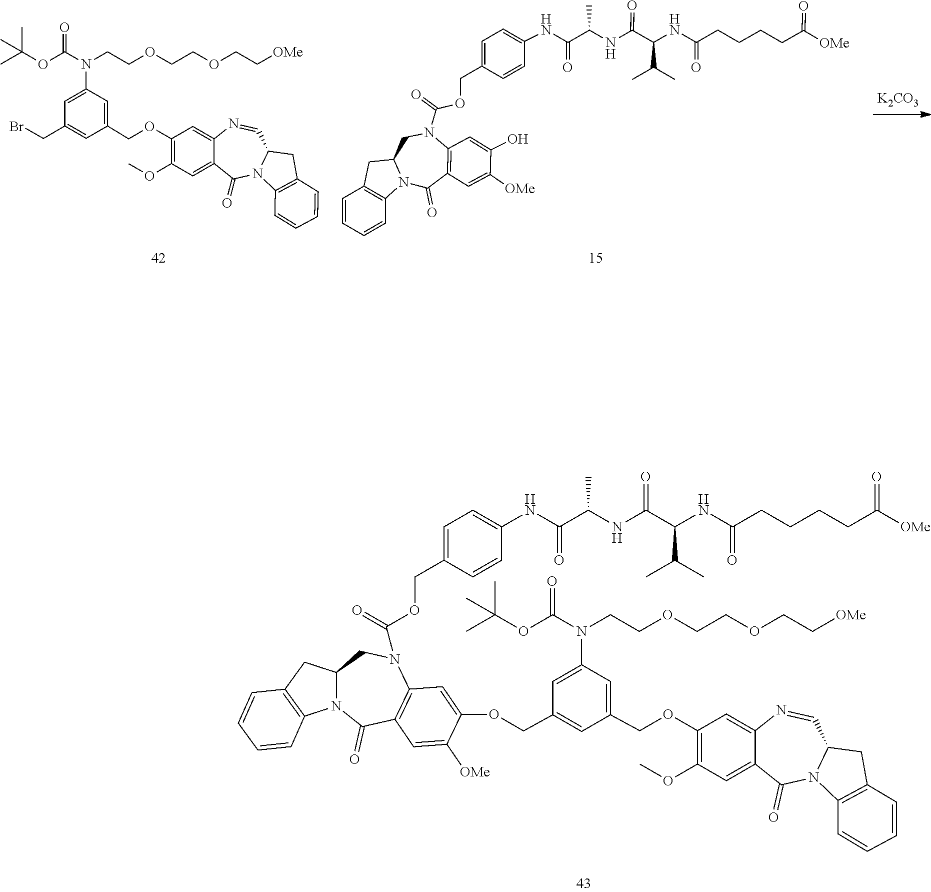

Compound 42 (94 mg, 0.127 mmol) and compound 15 (88 mg, 0.116 mmol) were dissolved in anhydrous dimethylacetamide (1157 μl). Potassium carbonate (32.0 mg, 0.231 mmol) was added and the reaction stirred overnight at room temperature. The reaction was precipitated with water, stirred for 5 minutes and filtered. The resulting solid was dissolved in dichloromethane with a small amount of methanol dried over anhydrous magnesium sulfate, filtered, and concentrated in vacuo. The crude material was purified via silica gel column (dichloromethane/methanol) to give compound 43 as a fluffy solid (110 mg, y=67%). MS (m/z): 1416.2 (M+1)⁺. UPLC=1.84 min (2.5 min method).

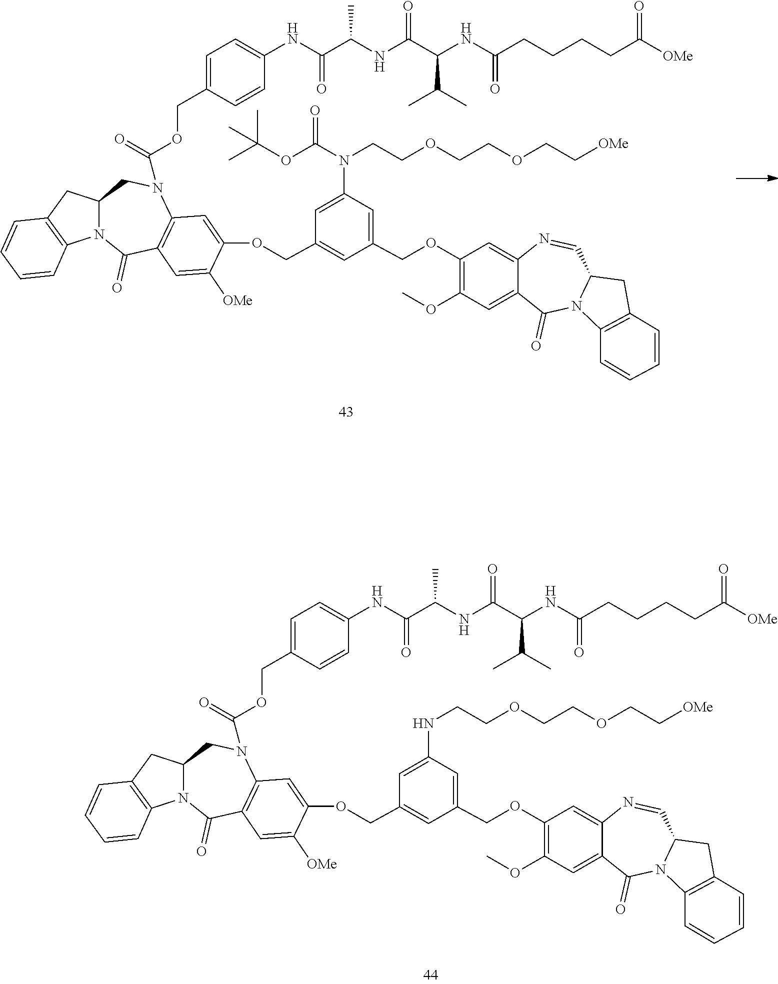 [SMILES: COCCOCCOCCN(C(=O)OC(C)(C)C)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](NC(=O)CCCCC(=O)OC)C(C)C)cc2)c1 ; COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](NC(=O)CCCCC(=O)OC)C(C)C)cc2)c1]

Compound 43 (110 mg, 0.078 mmol) was dissolved in anhydrous dichloromethane (700 μl) and cooled to OC in an ice bath. A freshly mixed solution of anhydrous dichloromethane (700 μl) and TFA (719 μl) was added. The reaction stirred at OC for 45 minutes. It was diluted reaction with dichloromethane, poured into an ice/saturated sodium bicarbonate solution, and then the separated organics were washed with brine, dried over magnesium sulfate, filtered and stripped. The crude was purified by silica gel chromatography in dichloromethane/methanol to give compound 44 (84 mg, y=82%). MS (m/z): 1316.2 (M+1)⁺. UPLC=1.72 min (2.5 min method).

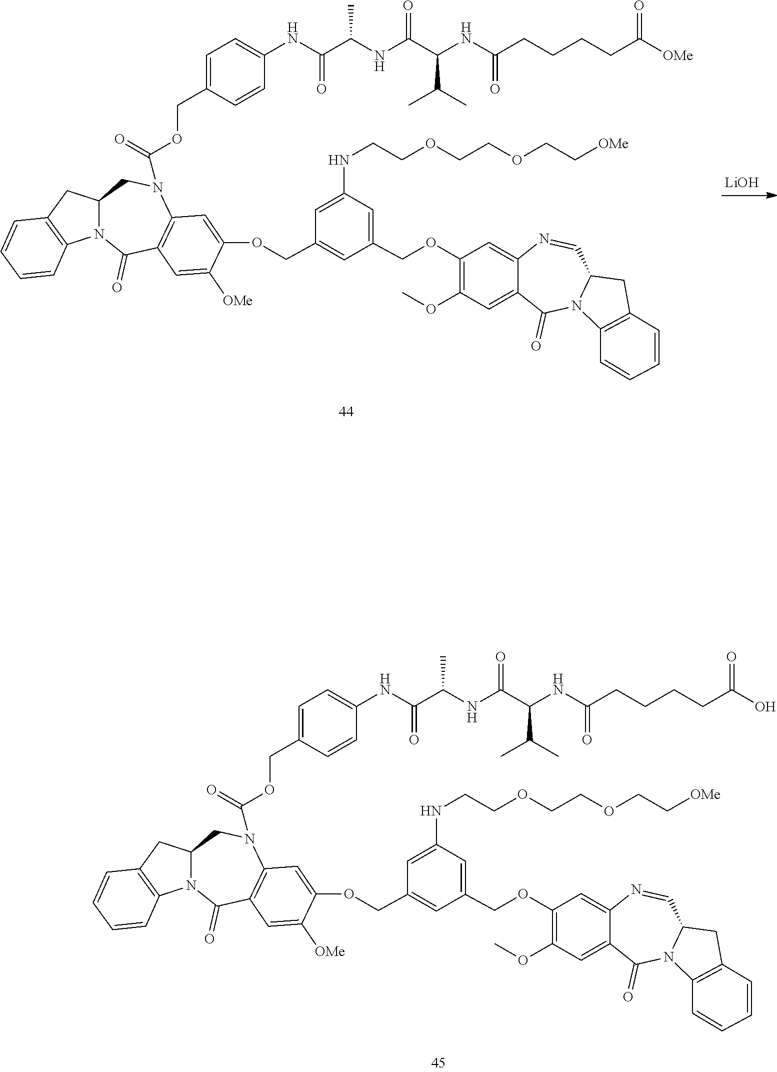 [SMILES: COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](NC(=O)CCCCC(=O)OC)C(C)C)cc2)c1 ; "LiOH" ; COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](NC(=O)CCCCC(=O)O)C(C)C)cc2)c1]

Compound 44 (84 mg, 0.064 mmol) was dissolved in anhydrous Tetrahydrofuran (2395 μl) and deionized water (798 μl). Lithium hydroxide (4.59 mg, 0.192 mmol) was added and the reaction stirred at room temperature for 1 hour. It was diluted with 30% methanol/dichloromethane and deionized water, then slowly acidified with 0.5 M HCl to pH˜3. The aqueous was extracted two more times with 30% methanol/dichloromethane. The combined organics were washed with water to pH=5, dried with magnesium sulfate, filtered over celite, concentrated and co-evaporated with anhydrous dichloromethane to get 89 mg crude compound 45. Assume 100% for next reaction mass. MS (m/z): 1302.4 (M+1)⁺. UPLC=1.64 min (2.5 min method).

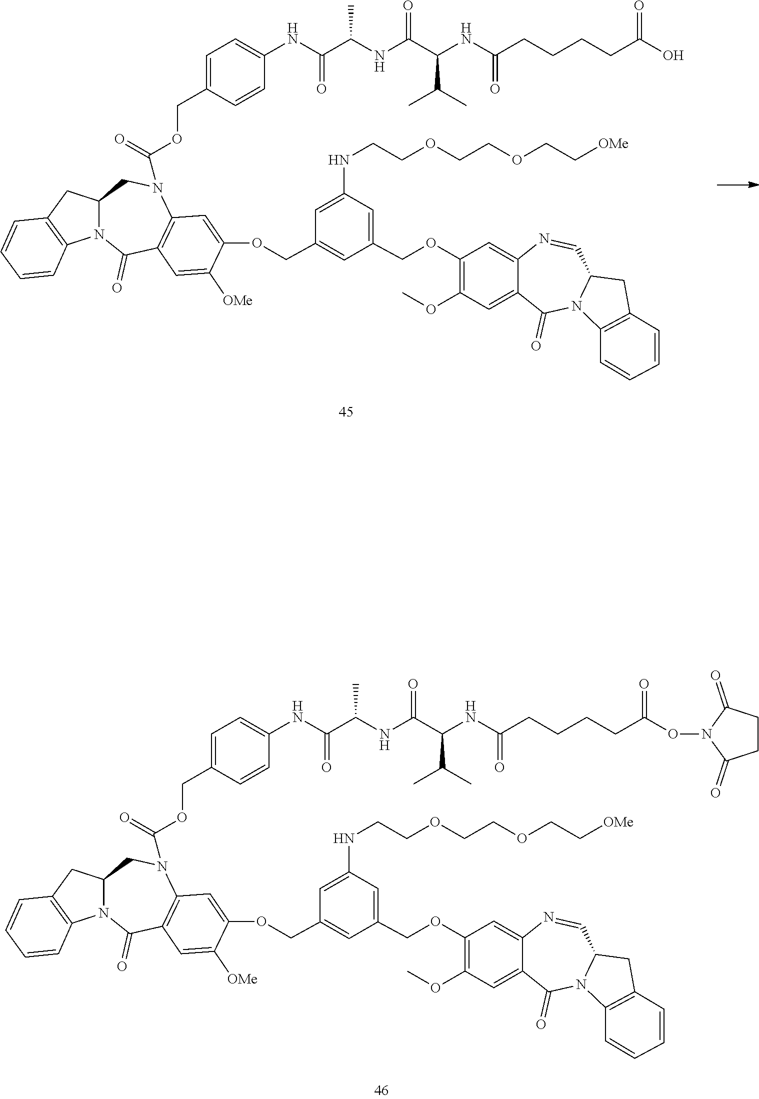 [SMILES: COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](NC(=O)CCCCC(=O)O)C(C)C)cc2)c1 ; COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](NC(=O)CCCCC(=O)ON3C(=O)CCC3=O)C(C)C)cc2)c1]

Compound 45 (83 mg, 0.064 mmol) was dissolved in anhydrous dichloromethane (1594 μl). N-hydroxy succinimide (22.02 mg, 0.191 mmol) and EDC.HCl (61.1 mg, 0.319 mmol) were added and the reaction stirred under argon for 50 minutes. It was diluted with dichloromethane and washed with water. The organic was dried over Magnesium sulfate, filtered and stripped. The crude solid was either used directly in the next fraction or purified by RP-HPLC (C18 Kromasil, acetonitrile/deionized water). The pure fractions were frozen and lyophilized to give compound 46 (51 mg, y=57%). MS (m/z): 1399.3 (M+1)⁺. UPLC=1.70 min (2.5 min method).

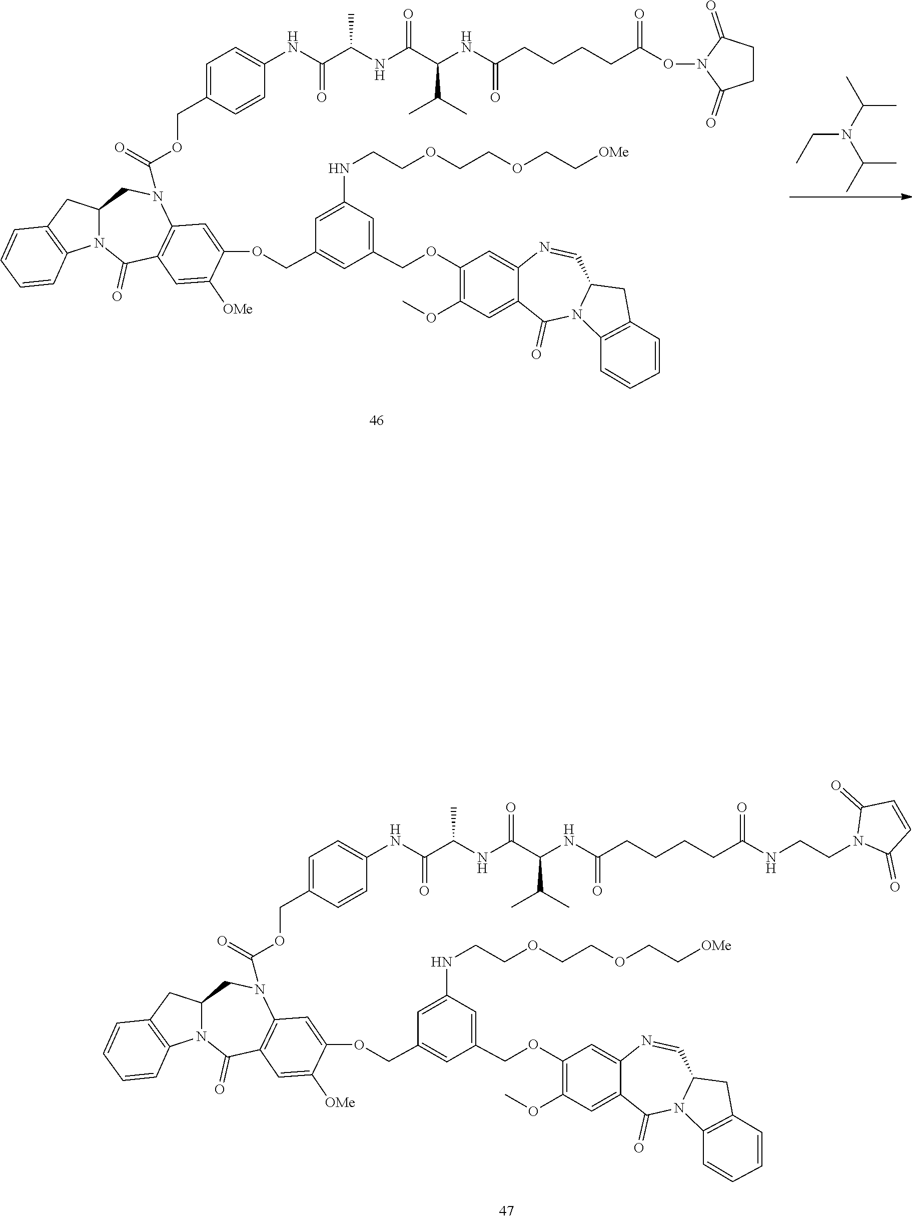

Compound 46 (53.7 mg, 0.038 mmol) was dissolved in anhydrous dichloromethane (1536 μl). 1-(2-aminoethyl)-1H-pyrrole-2,5-dione hydrochloride (8.02 mg, 0.042 mmol) was added followed by anhydrous N-ethyl-N-isopropylpropan-2-amine (13.38 μl, 0.077 mmol). The reaction was monitored by UPLC and after 1.5 hours was concentrated to dryness. The crude solid was dissolved in acetonitrile/deionized water/Tetrohydrofuran and a few drops of formic acid and purified by RP-HPLC (C18, water/acetonitrile). The pure fractions were combined and frozen and lyophilized to yield pure compound 47 (20.4 mg, 37%). MS (m/z): 1424.7 (M+1)⁺. UPLC=1.65 min (2.5 min method).

### Example 6. Synthesis of Compound 55 and Compound 56

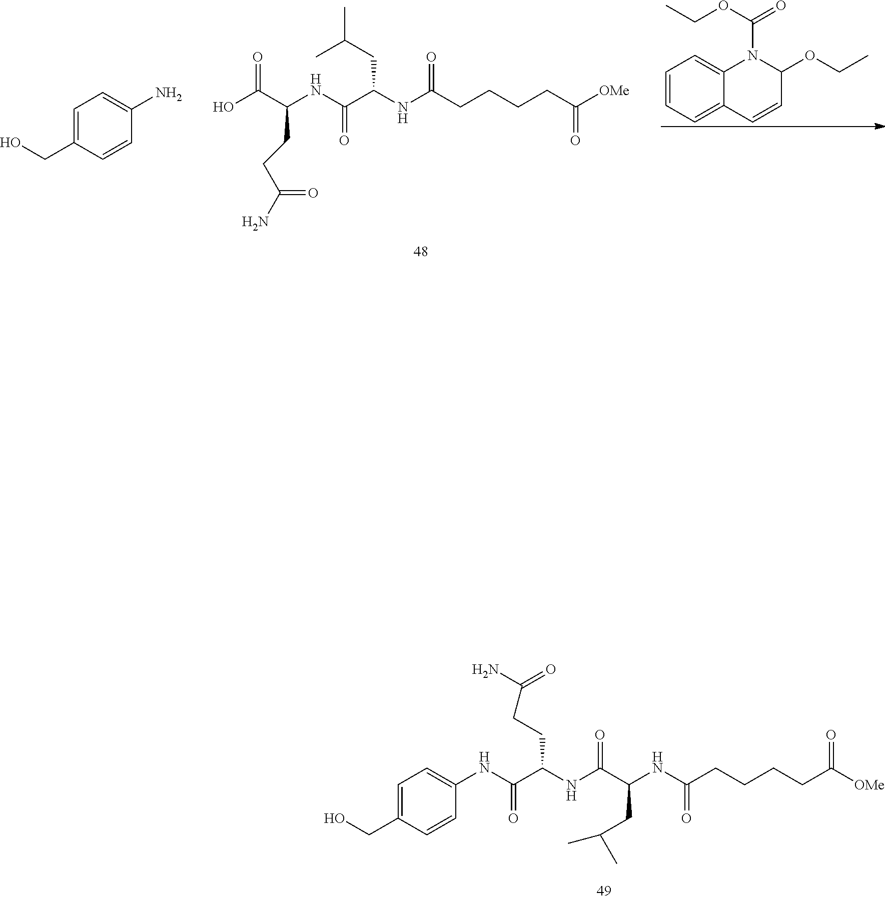 [SMILES: Nc1ccc(CO)cc1 ; COC(=O)CCCCC(=O)N[C@@H](CC(C)C)C(=O)N[C@@H](CCC(N)=O)C(=O)O ; CCOC(=O)N1c2ccccc2C=CC1OCC ; COC(=O)CCCCC(=O)N[C@@H](CC(C)C)C(=O)N[C@@H](CCC(N)=O)C(=O)Nc1ccc(CO)cc1]

To a solution of (6-methoxy-6-oxohexanoyl)-L-leucyl-L-glutamine (0.83 g, 2.067 mmol) and (4-aminophenyl)methanol (0.306 g, 2.481 mmol) in anhydrous dichloromethane (9.19 ml) and anhydrous methanol (4.59 ml) (bright yellow solution) was added EEDQ (1.023 g, 4.13 mmol) and the reaction mixture was stirred for 18 hours at ambient temperature under nitrogen. The reaction mixture was concentrated and the resulting residue was triturated with ethyl acetate. The off-white solid was filtered, washed with ethyl acetate and dried to give methyl 6-(((S)-1-(((S)-5-amino-1-((4-(hydroxymethyl)phenyl)amino)-1,5-dioxopentan-2-yl)amino)-4-methyl-1-oxopentan-2-yl)amino)-6-oxohexanoate (0.5 g, y=48%) as a white solid. MS (m/z): 508.05 (M+1)⁺. LC=3.72 min

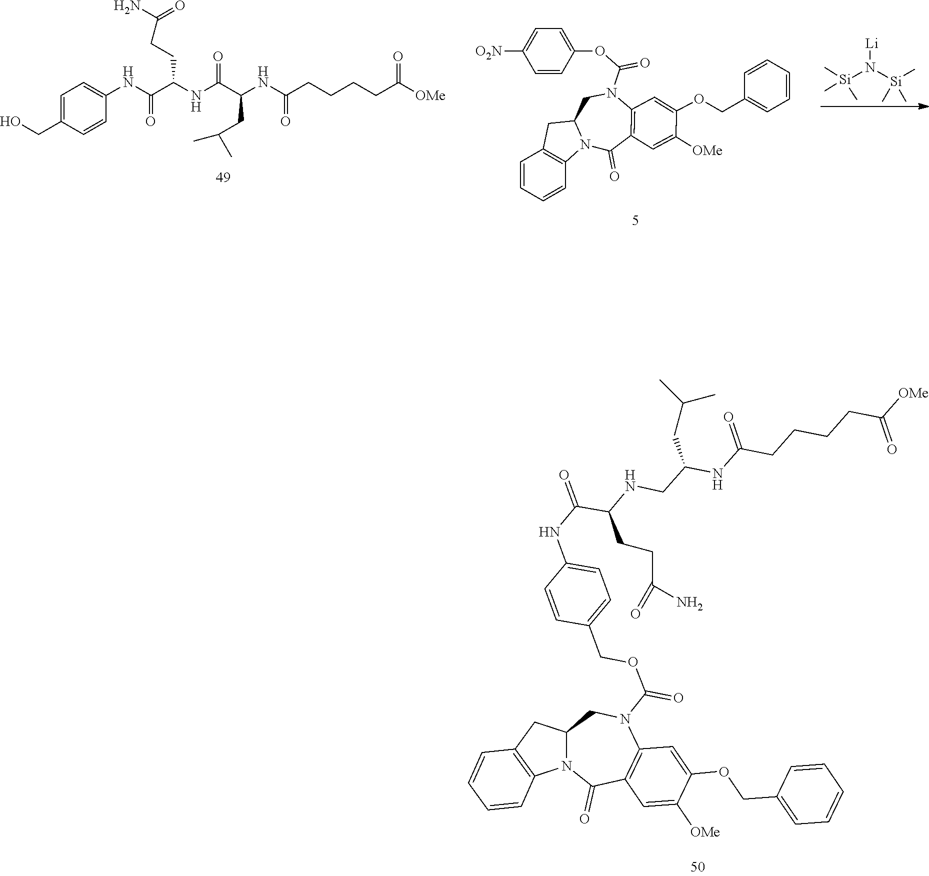

To a solution of methyl 6-(((S)-1-(((S)-5-amino-1-((4-(hydroxymethyl)phenyl)amino)-1,5-dioxopentan-2-yl)amino)-4-methyl-1-oxopentan-2-yl)amino)-6-oxohexanoate (450 mg, 0.888 mmol) in anhydrous tetrahydrofuran (1.68 ml) and anhydrous N,N-Dimethylacetamide (3.36 ml) was added lithium bis(trimethylsilyl)amide (1M in Tetrahydrofuran, 0.888 ml, 0.888 mmol) at 0° C. The clear yellow reaction was stirred for 15 minutes then compound 5 (408 mg, 0.740 mmol) in anhydrous Tetrahydrofuran (1.68 ml) was added. The reaction mixture was stirred at 0° C. and allowed to warm to room temperature over 18 hours. The mixture was quenched reaction at 0° C. with saturated aqueous ammonium chloride solution and extracted with dichloromethane (3×50 mL). The combined organic layers were washed with water (2×50 mL) and then dried over anhydrous magnesium sulfate, filtered and concentrated. The crude material was purified by ISCO (24 g silica column, methanol/dichloromethane) to give compound 50 (95 mg, 14% yield) as a white solid. MS (m/z): 919.8 (M+1)⁺. UPLC=5.54 min (10 min method).

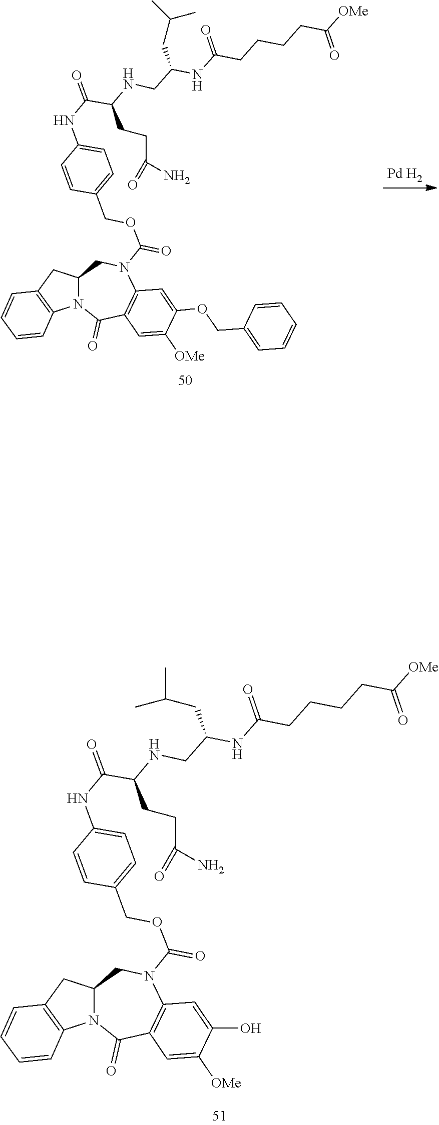 [SMILES: COC(=O)CCCCC(=O)N[C@H](CN[C@@H](CCC(N)=O)C(=O)Nc1ccc(COC(=O)N2C[C@@H]3Cc4ccccc4N3C(=O)c3cc(OC)c(OCc4ccccc4)cc32)cc1)CC(C)C ; "Pd H2" ; COC(=O)CCCCC(=O)N[C@H](CN[C@@H](CCC(N)=O)C(=O)Nc1ccc(COC(=O)N2C[C@@H]3Cc4ccccc4N3C(=O)c3cc(OC)c(O)cc32)cc1)CC(C)C]

A solution of 4-((S)-5-amino-2-((S)-2-(6-methoxy-6-oxohexanamido)-4-methylpentanamido)-5-oxopentanamido)benzyl (S)-9-(benzyloxy)-8-methoxy-6-oxo-12a,13-dihydro-6H-benzo[5,6][1,4]diazepino[1,2-a]indole-11(12H)-carboxylate (91 mg, 0.099 mmol) in anhydrous methanol (1.5 ml) was degassed and palladium on carbon 10% (10.54 mg, 0.099 mmol) was added. The mixture was stirred under hydrogen balloon (1 atm) at room temperature for four hours after which it was filtered through celite, rinsing with methanol. The filtrate was evaporated to obtain DP (83 mg, 100%) as a white solid. MS (m/z): 829.6 (M+1)⁺827.5 (M−1) UPLC=4.25 min (10 min method).

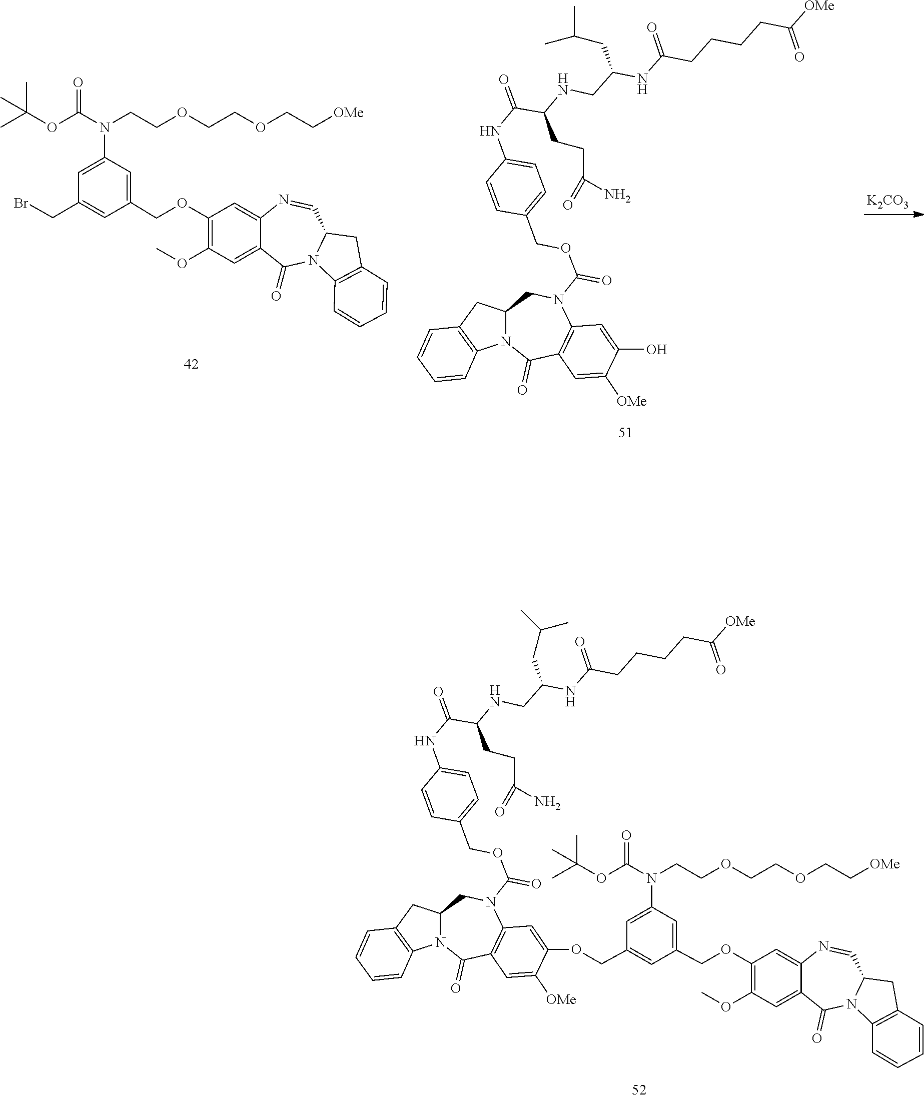 [SMILES: COCCOCCOCCN(C(=O)OC(C)(C)C)c1cc(CBr)cc(COc2cc3c(cc2OC)C(=O)N2c4ccccc4C[C@H]2C=N3)c1 ; COC(=O)CCCCC(=O)N[C@H](CN[C@@H](CCC(N)=O)C(=O)Nc1ccc(COC(=O)N2C[C@@H]3Cc4ccccc4N3C(=O)c3cc(OC)c(O)cc32)cc1)CC(C)C ; "K2CO3" ; COCCOCCOCCN(C(=O)OC(C)(C)C)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](CCC(N)=O)NC[C@H](CC(C)C)NC(=O)CCCCC(=O)OC)cc2)c1]

Compound 42 (215 mg, 0.291 mmol) and Compound 51 (201 mg, 0.242 mmol) were dissolved in anhydrous dimethylacetamide (2425 μl). Potassium carbonate (67.0 mg, 0.485 mmol) was added and the reaction stirred overnight at room temperature. The reaction was precipitated with water, stirred for 5 minutes and filtered. The resulting yellow solid was dissolved in dichloromethane/5% methanol and washed with water. The organic was dried over anhydrous magnesium sulfate, filtered and stripped. The material was purified by silica gel chromatography (dichloromethane/methanol) to give the desired product, compound 52 (323 mg, y=64%). MS (m/z): 1487.6 (M+1)⁺. UPLC=1.77 min (2.5 min method).

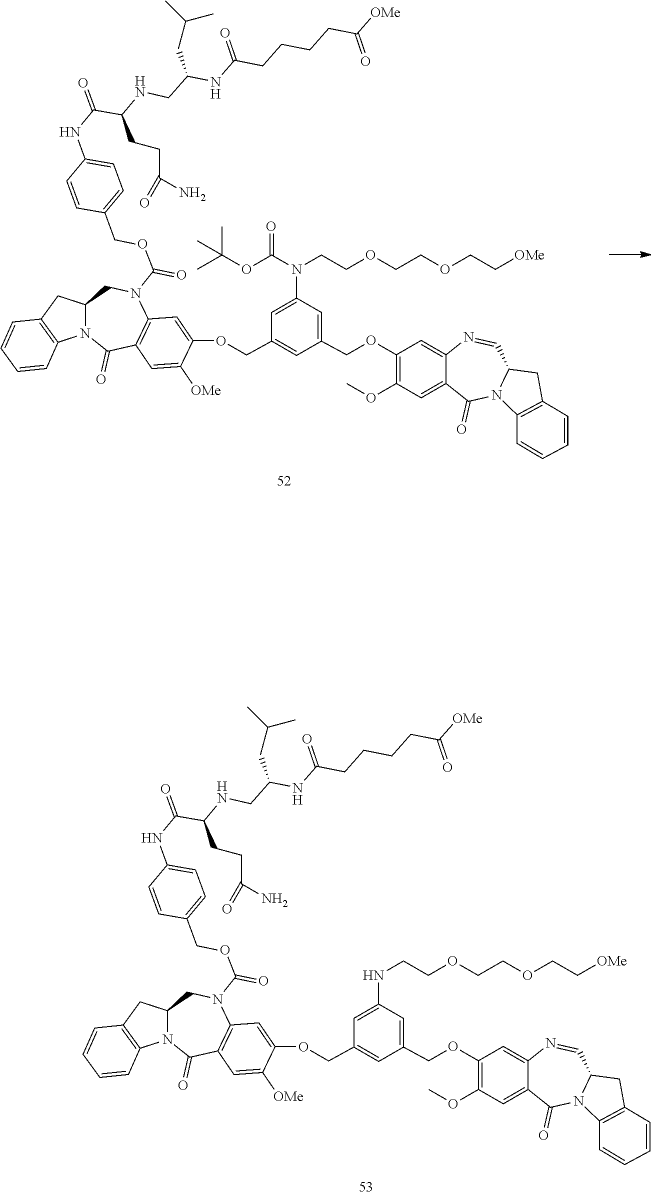 [SMILES: COCCOCCOCCN(C(=O)OC(C)(C)C)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](CCC(N)=O)NC[C@H](CC(C)C)NC(=O)CCCCC(=O)OC)cc2)c1 ; COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](CCC(N)=O)NC[C@H](CC(C)C)NC(=O)CCCCC(=O)OC)cc2)c1]

Compound 53 was prepared similarly as Compound 44. The crude product was purified by silica gel chromatography (dichloromethane/methanol). MS (m/z): 1387.7 (M+1)⁺. UPLC=1.8 (2.5 min method).

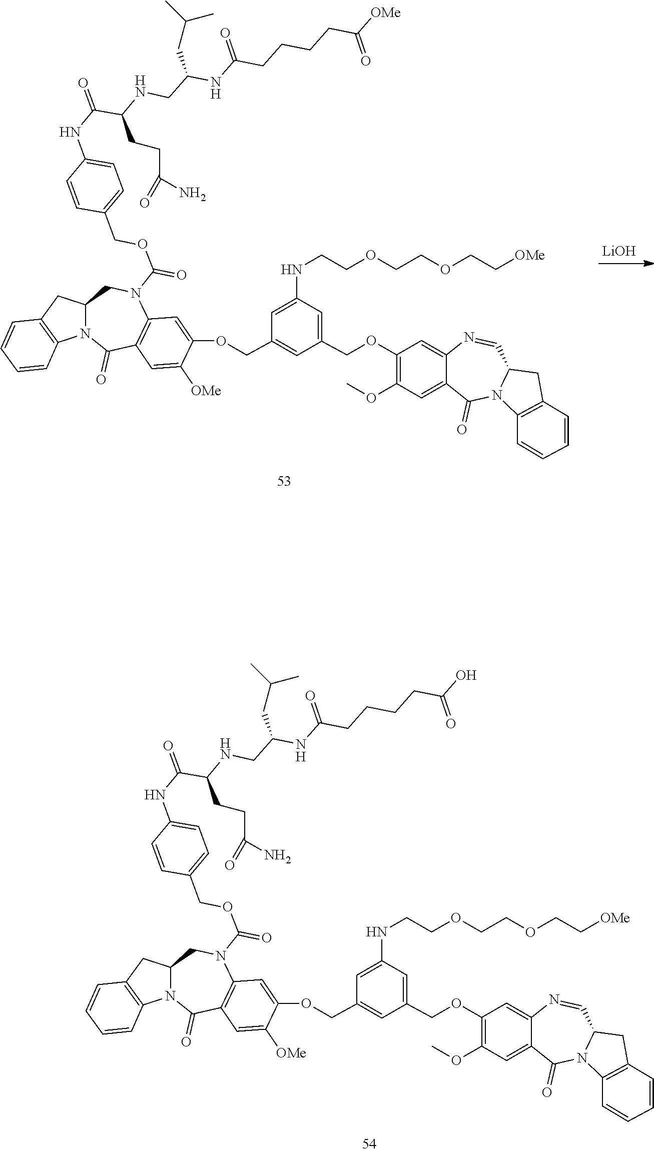 [SMILES: COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](CCC(N)=O)NC[C@H](CC(C)C)NC(=O)CCCCC(=O)OC)cc2)c1 ; "LiOH" ; COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](CCC(N)=O)NC[C@H](CC(C)C)NC(=O)CCCCC(=O)O)cc2)c1]

Compound 54 was prepared similarly as Compound 45. The crude product was purified by silica gel chromatography (dichloromethane/methanol) (117 mg, y=45.5%). MS (m/z): 1373.9 (M+1)⁺. UPLC=1.71 (2.5 min method).

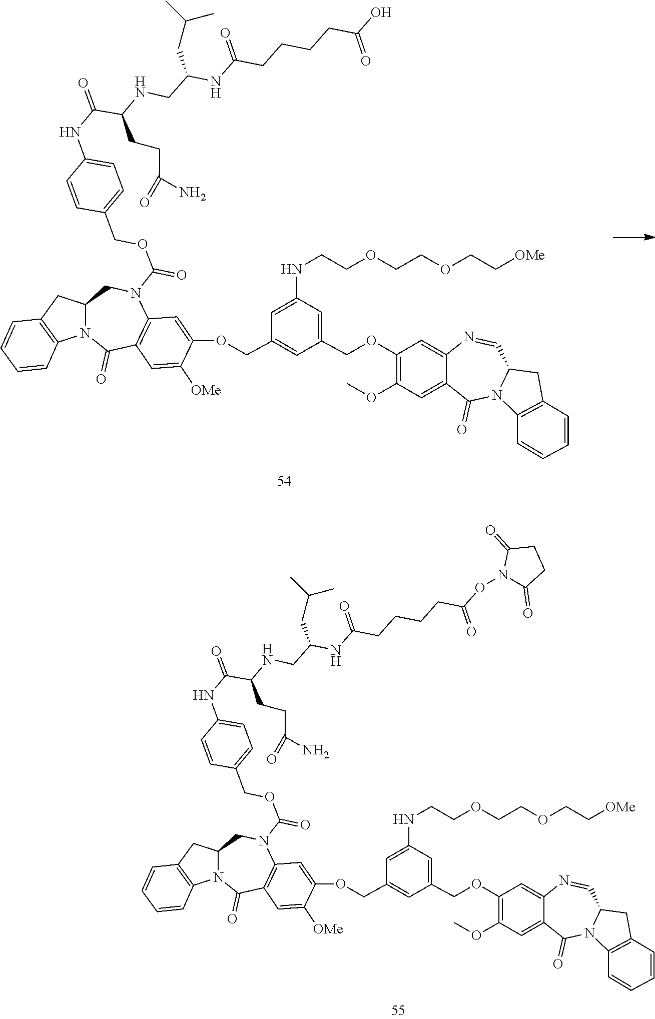 [SMILES: COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](CCC(N)=O)NC[C@H](CC(C)C)NC(=O)CCCCC(=O)O)cc2)c1 ; COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](CCC(N)=O)NC[C@H](CC(C)C)NC(=O)CCCCC(=O)ON3C(=O)CCC3=O)cc2)c1]

Compound 55 was prepared similarly as Compound 46. The crude product used directly in the next reaction or purified by RPHPLC (water/acetonitrile) (34.5 mg, y=49%). MS (m/z): 1470.8 (M+1)⁺. UPLC=1.74 (2.5 min method).

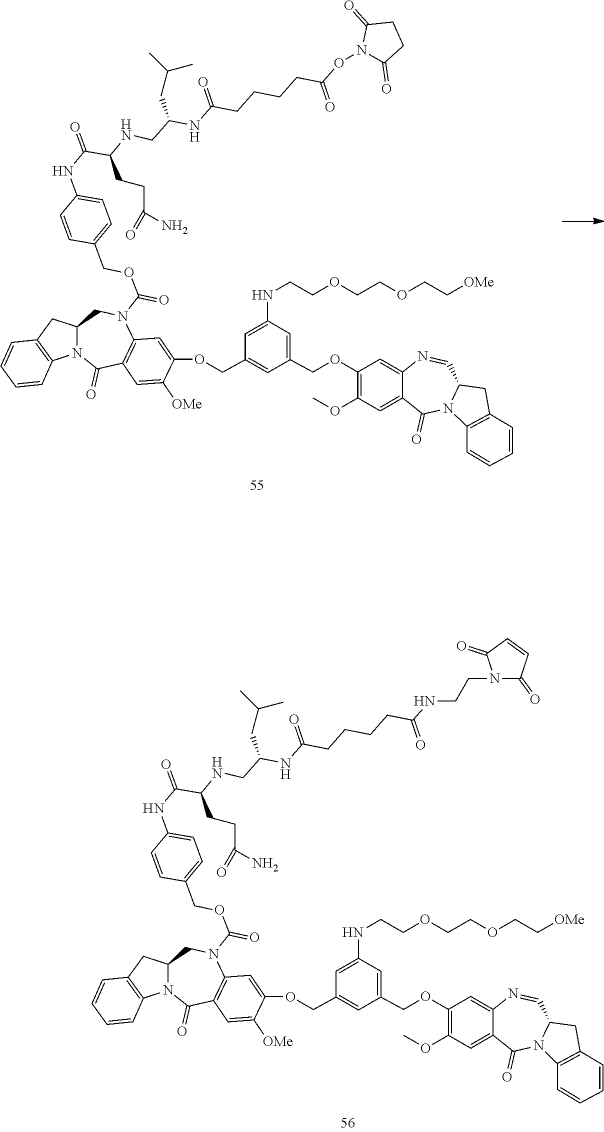 [SMILES: COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](CCC(N)=O)NC[C@H](CC(C)C)NC(=O)CCCCC(=O)ON3C(=O)CCC3=O)cc2)c1 ; COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](CCC(N)=O)NC[C@H](CC(C)C)NC(=O)CCCCC(=O)NCCN3C(=O)C=CC3=O)cc2)c1]

Compound 56 was prepared similarly as Compound 47. The crude product was purified by RPHPLC (water/acetonitrile) (26 mg, y=47%). MS (m/z): 1495.9 (M+1)⁺. UPLC=1.71 (2.5 min method).

### Example 7. Synthesis of Compound 65

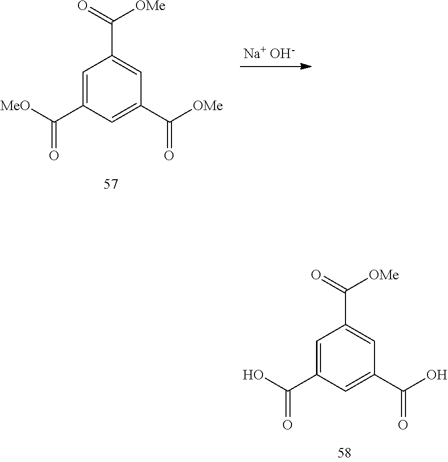 [SMILES: "Na+ OH-" ; COC(=O)c1cc(C(=O)OC)cc(C(=O)OC)c1 ; COC(=O)c1cc(C(=O)O)cc(C(=O)O)c1]

Sodium Hydroxide (2.065 g, 51.6 mmol) was added to a stirred solution of trimethyl benzene-1,3,5-tricarboxylate (6.2 g, 24.58 mmol) in methanol (82 ml) and water (16.39 ml). The reaction mixture was refluxed (oil bath at 85° C.) under argon for 3 hours and a white precipitate formed. The reaction was cooled to room temperature and diluted with water until all the solids were dissolved. The solution was acidified to pH-2-3 with aqueous 5N HCl. The methanol was removed in vacuo and the resulting aqueous was extracted with ethyl acetate three times. The combined organic was dried over sodium sulfate, filtered and stripped. The white product was dissolved in hot ethyl acetate and then allowed to cool. The solution was filtered (precipitate was by-product) and the filtrate was evaporated to give desired compound 58 (4.34 g, y=79%). MS (m/z): 225.0 (M+1)⁺ and 224.0 (M−1)⁻. UPLC=1.07 min (2.5 min method). ¹H NMR (400 MHz, DMSO-d6): δ 13.62 (bs, 2H), 8.65 (s, 3H), 3.93 (s, 3H).

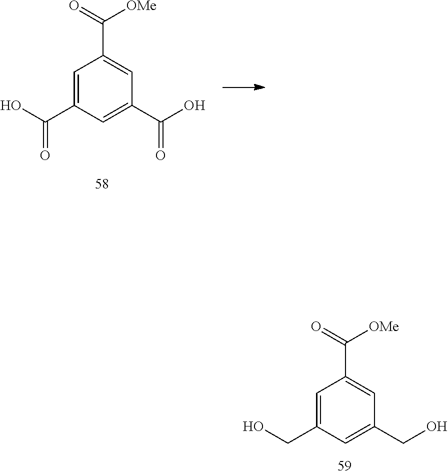 [SMILES: COC(=O)c1cc(C(=O)O)cc(C(=O)O)c1 ; COC(=O)c1cc(CO)cc(CO)c1]

Compound 58 (1.94 g, 8.65 mmol) was dissolved in THF (34.6 ml) and cooled to 0° C. BH3.DMS (2M in THF, 17.31 ml, 34.6 mmol) was added slowly under argon causing vigorous bubbling. The reaction warmed to room temperature and stirred overnight. It was slowly quenched with methanol until the formation of bubbles lessened in intensity and then water was added until the evolution of gas ceased and all solids had dissolved. The solution was extracted with ethyl acetate twice. The combined organics were washed sequentially with 75 ml of ˜3% H₂O₂ solution, 150 ml of 0.5M aq. citric acid solution and brine. The organic was dried, concentrated and purified by silica gel chromatography (hexanes/ethyl acetate) to give compound 59 (0.82 g, y=32%). ¹H NMR (400 MHz, DMSO-d6): δ 7.81 (s, 2H), 7.52 (s, 1H), 5.33 (bs, 2H), 4.56 (s, 4H), 3.86 (s, 3H).

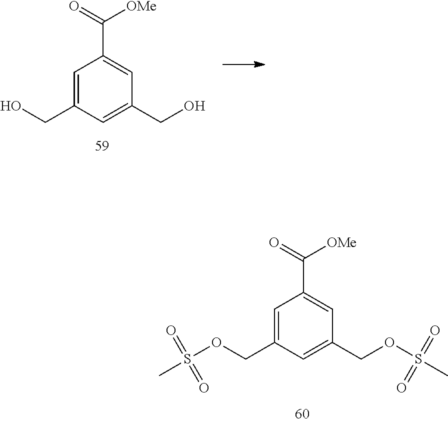

Compound 59 (0.82 g, 4.18 mmol) was dissolved in anhydrous dichloromethane (41.8 ml) and cooled to −5° C. in an acetone/ice bath. Triethylamine (2.91 ml, 20.90 mmol) was added followed by the addition of methanesulfonic anhydride (1.820 g, 10.45 mmol) and the resulting mixture was stirred at −5° C. for 1.5 hours. It was diluted with cold ethyl acetate and water and extracted twice more with ethyl acetate. The combined organics were washed with water, dried over anhydrous magnesium sulfate, filtered and concentrated (<25 C) in vacuo to give compound 60 (1.12 g, y=76%) that was used directly in the next reaction.

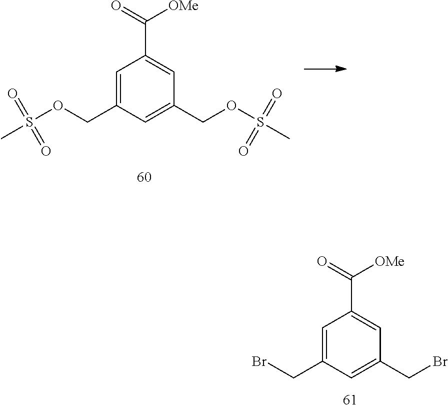

Compound 60 (1.12 g, 3.18 mmol) was dissolved in anhydrous dimethylformamide (15.89 ml) and sodium bromide (1.635 g, 15.89 mmol) was added. The reaction stirred at room temperature overnight. It was diluted with water and extracted with ethyl acetate. The organic was washed with water and dried over anhydrous magnesium sulfate, filtered and stripped. The crude material was placed on the high vacuum until dry to give compound 61 (0.97 g, y=94%) that was used without further purification. UPLC=1.66 min (2.5 min method).

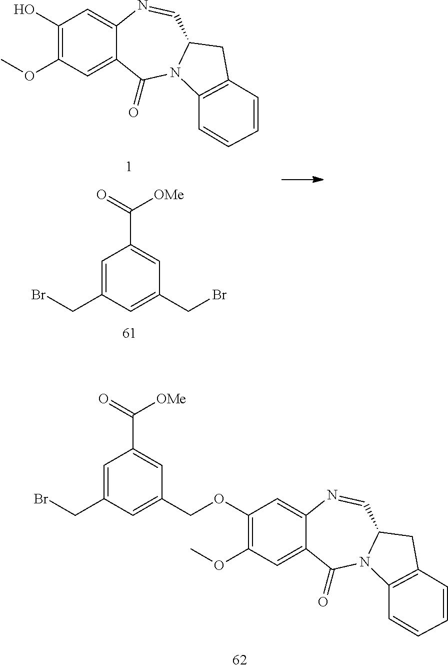 [SMILES: COc1cc2c(cc1O)N=C[C@@H]1Cc3ccccc3N1C2=O ; COC(=O)c1cc(CBr)cc(CBr)c1 ; COC(=O)c1cc(CBr)cc(COc2cc3c(cc2OC)C(=O)N2c4ccccc4C[C@H]2C=N3)c1]

Compound 1 (0.126 g, 0.429 mmol) and compound 61 (0.967 g, 3.00 mmol) were dissolved in anhydrous dimethylformamide (4.29 ml) and Potassium carbonate (0.148 g, 1.073 mmol) was added. After 1 hour and 20 minutes all of compound 1 had been consumed and the reaction was diluted with ethyl acetate and washed with saturated ammonium chloride, water, and brine. The organic was dried over magnesium sulfate, filtered, and stripped. Pure compound 62 was isolated by silica gel chromatography (133 mg, y=58%). UPLC=1.71 min (2.5 min method).

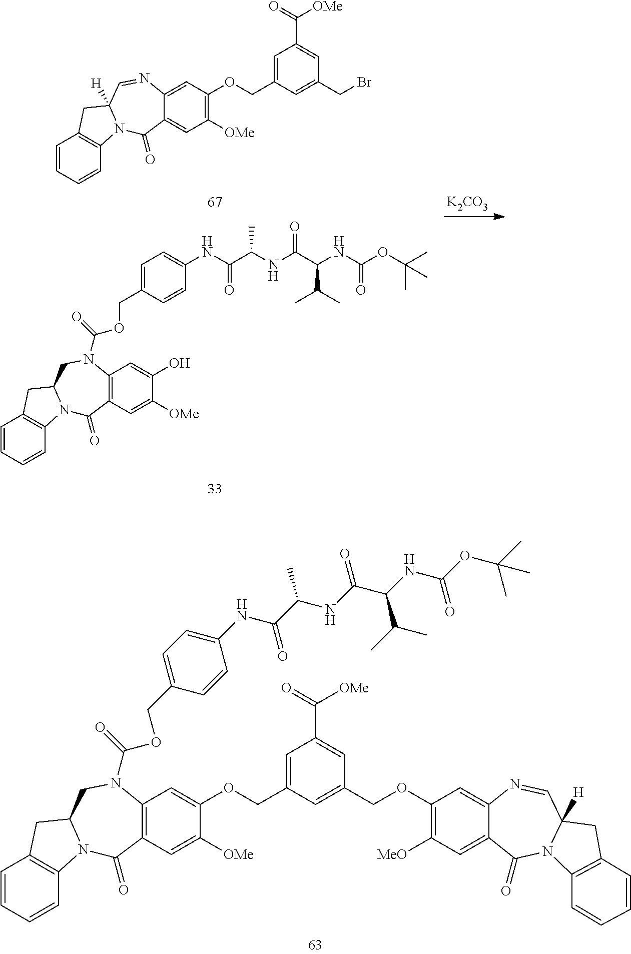 [SMILES: COC(=O)c1cc(CBr)cc(COc2cc3c(cc2OC)C(=O)N2c4ccccc4C[C@H]2C=N3)c1 ; "K2CO3" ; COc1cc2c(cc1O)N(C(=O)OCc1ccc(NC(=O)[C@H](C)NC(=O)[C@@H](NC(=O)OC(C)(C)C)C(C)C)cc1)C[C@@H]1Cc3ccccc3N1C2=O ; COC(=O)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](NC(=O)OC(C)(C)C)C(C)C)cc2)c1]

Compound 33 (63.6 mg, 0.089 mmol) and compound 62 (63 mg, 0.098 mmol) were dissolved in anhydrous dimethylacetamide (888 μl). Potassium carbonate (24.54 mg, 0.178 mmol) was added and the reaction stirred overnight under argon. The reaction was precipitated with water, stirred for 5 minutes and filtered. The resulting white solid was dissolved in dichloromethane, transferred to a separatory funnel, washed with water, dried over anhydrous magnesium sulfate, filtered and stripped. The crude was purified via column to give compound 63 (79 mg, y=76%). MS (m/z): 1171.0 (M+1)⁺. UPLC=1.89 min (2.5 min method).

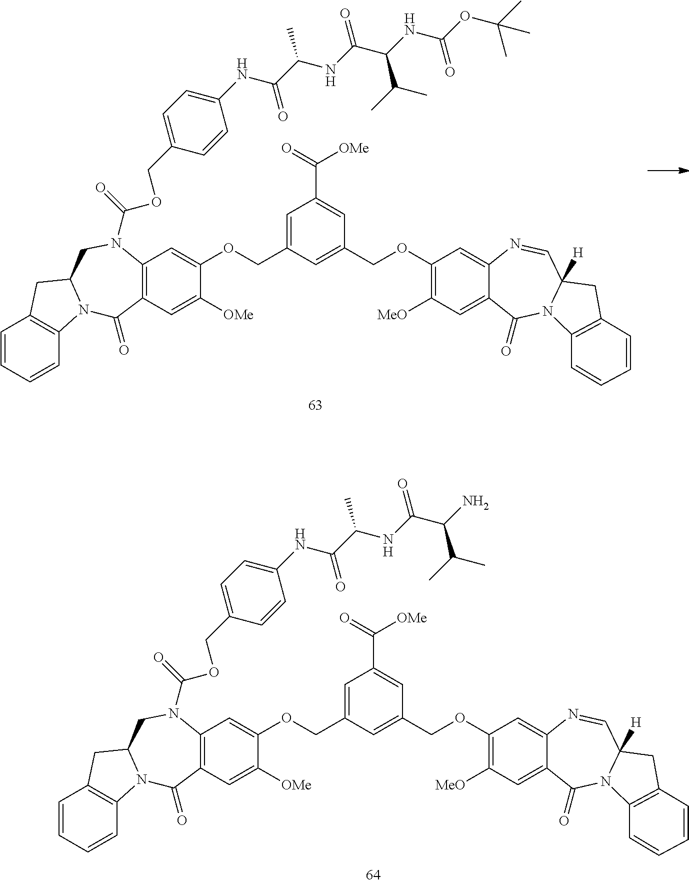 [SMILES: COC(=O)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](NC(=O)OC(C)(C)C)C(C)C)cc2)c1 ; COC(=O)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](N)C(C)C)cc2)c1]

Compound 64 was prepared similarly as Compound 35. The crude material was used directly without purification (54 mg, y=75%). MS (m/z): 1171.1 (M+1)⁺. UPLC=1.51 min (2.5 min method).

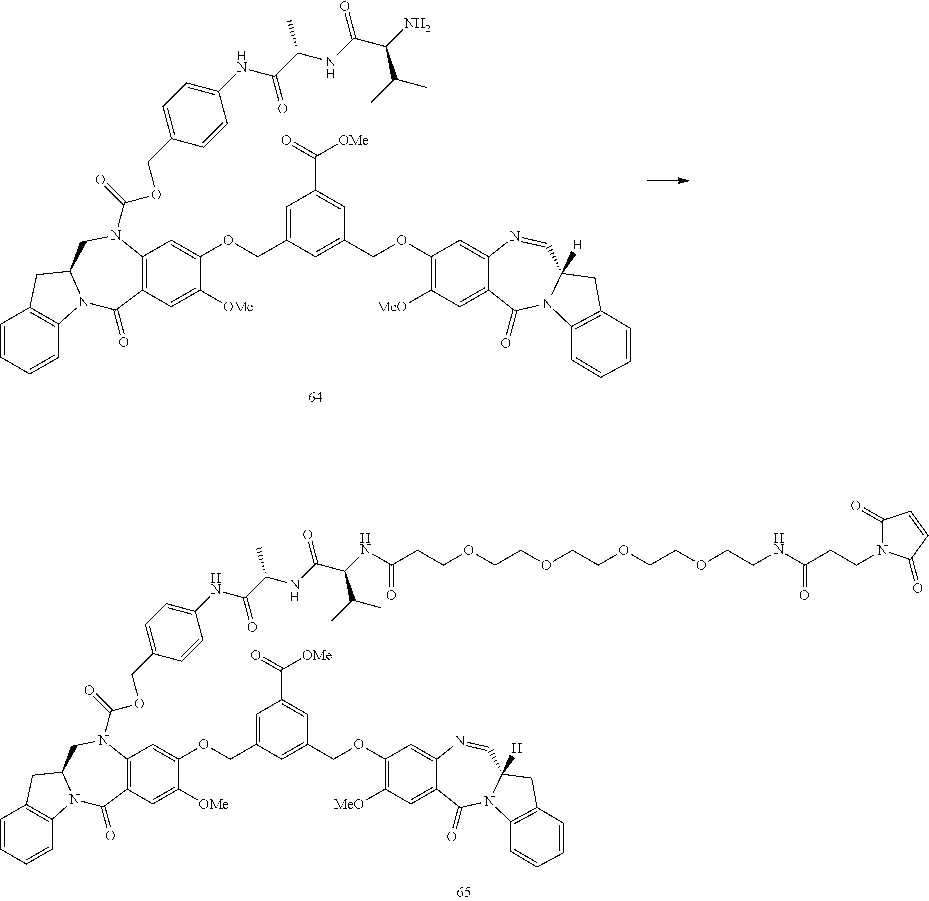 [SMILES: COC(=O)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](N)C(C)C)cc2)c1 ; COC(=O)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCc2ccc(NC(=O)[C@H](C)NC(=O)[C@@H](NC(=O)CCOCCOCCOCCOCCNC(=O)CCN3C(=O)C=CC3=O)C(C)C)cc2)c1]

Compound 64 (54 mg, 0.050 mmol) and 1-(2,5-dioxo-2,5-dihydro-1H-pyrrol-1-yl)-3-oxo-7,10,13,16-tetraoxa-4-azanonadecan-19-oic acid (21.01 mg, 0.050 mmol) were dissolved in anhydrous dichloromethane (3154 μl). EDC (9.67 mg, 0.050 mmol) was added and the reaction degassed and stirred at room temperature under argon. At 1 hour the reaction was diluted with dichloromethane and washed with water and brine. The organic was dried with magnesium sulfate, filtered and stripped. The crude material was dissolved in acetonitrile and Tetrahydrofurna plus a few drops of formic acid and purified by semi prep RP-HPLC (C18, water/acetonitrile). The pure fractions were frozen and lyophilized to provide compound 65 (29.5 mg, y=40%). MS (m/z): 1469.6 (M+1)⁺. UPLC=1.68 min (2.5 min method).

### Example 8. Synthesis of Compound 73

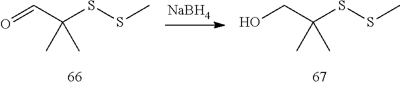

To a solution of 2-methyl-2-(methyldisulfanyl)propanal (1 g, 6.66 mmol) in anhydrous methanol (44.4 ml) was added Sodium borohydride (0.252 g, 6.66 mmol). The reaction stirred 90 minutes at room temperature upon which it was quenched with aqueous hydrochloric acid (0.5M) and diluted with ethyl acetate (100 ml). Aqueous saturated sodium bicarbonate solution was added until pH ˜10 and then extracted with ethyl acetate (2×50 ml). The organic extracts were combined, washed with brine, dried over anhydrous magnesium sulfate, filtered and concentrated. The crude was carried on without further purification, assuming 100% yield.

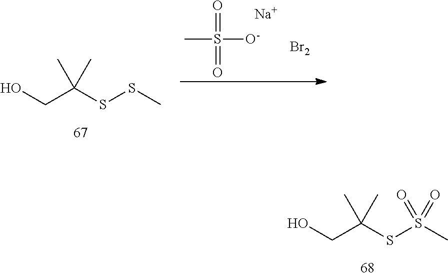

To a cooled solution of 2-methyl-2-(methyldisulfanyl)propan-1-ol (1.2 g, 7.88 mmol) in anhydrous dichloromethane (52.5 ml) was added sodium methanesulfonate (4.65 g, 39.4 mmol) and dibromine (1.009 ml, 19.70 mmol) at 0° C. After stirring at ambient temperature for 90 minutes the reaction mixture was filtered through celite and rinsed with dichloromethane. The filtrate was concentrated and purified by ISCO (40 g silica column, ethyl acetate/hexanes) to give S-(1-hydroxy-2-methylpropan-2-yl) methanesulfonothioate (1.1 g, y=76%). ¹H NMR (400 Hz, d₆-DMSO): δ 5.39 (t, J=5.6 Hz, 1H), 3.54 (d, J=5.6 Hz, 2H), 3.50 (s, 3H), 1.44 (s, 6H)

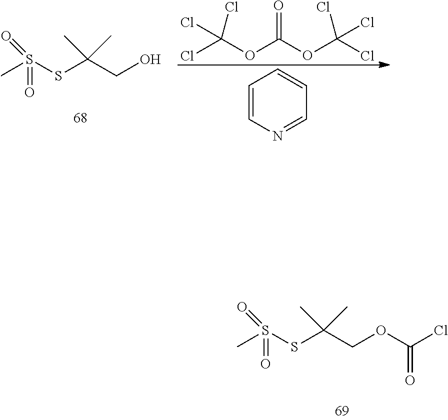

To a solution of S-(1-hydroxy-2-methylpropan-2-yl) methanesulfonothioate (47 mg, 0.255 mmol) and triphosgene (26.5 mg, 0.089 mmol) in anhydrous dichloromethane (1.27 ml) was added pyridine (19.60 μl, 0.242 mmol). The reaction stirred at ambient temperature for four hours after which it was diluted with dichloromethane and washed with water. The organic layer was dried with anhydrous magnesium sulfate, filtered and concentrated to get a clear colorless oil. The crude compound 69 was carried on without further purification, assuming 100% yield.

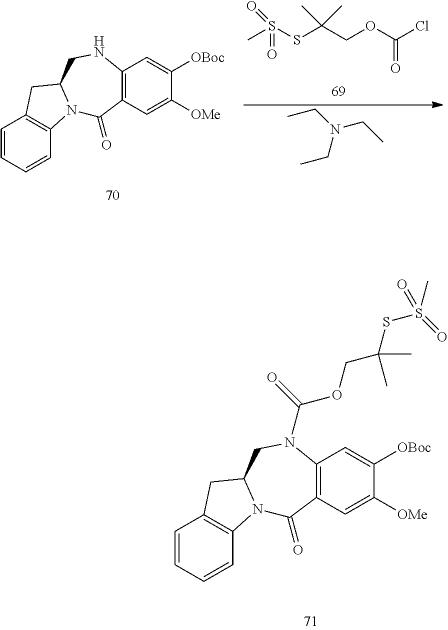

To a solution of (S)-tert-butyl (8-methoxy-6-oxo-11,12,12a,13-tetrahydro-6H-benzo[5,6][1,4]diazepino[1,2-a]indol-9-yl) carbonate (40 mg, 0.101 mmol) and S-(1-((chlorocarbonyl)oxy)-2-methylpropan-2-yl) methanesulfonothioate (62.2 mg, 0.252 mmol) in anhydrous 1,2-Dichloroethane (1.00 ml) was added triethylamine (56.3 μl, 0.404 mmol). The mixture stirred at ambient temperature for 30 minutes and was then diluted with dichloromethane. The organic layer was washed with brine, dried and concentrated. The crude material was purified by silica gel chromatography in ethyl acetate/hexanes to obtain Compound 71 as a white solid (50 mg, y=82%). MS (m/z): 607.6 (M+1)⁺. UPLC=1.81 min (2.5 min method).

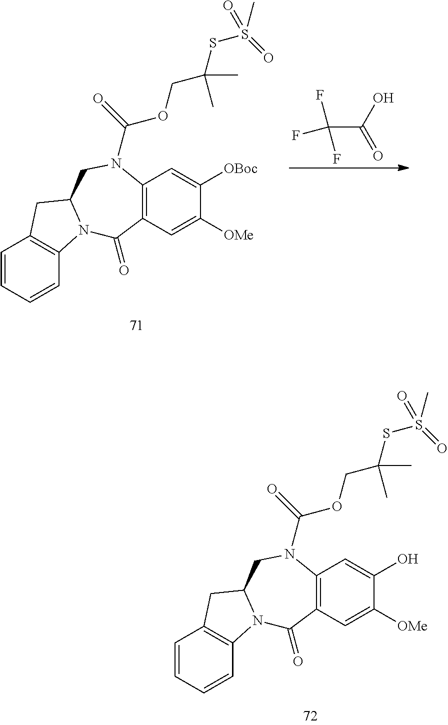

To a solution 2-methyl-2-((methylsulfonyl)thio)propyl (S)-9-((tert-butoxycarbonyl)oxy)-8-methoxy-6-oxo-12a,13-dihydro-6H-benzo[5,6][1,4]diazepino[1,2-a]indole-11(12H)-carboxylate (0.910 g, 1.5 mmol) in anhydrous dichloromethane (15.00 ml) was slowly added trifluoroacetic acid (1.733 ml, 22.50 mmol). The mixture was allowed to stir at ambient temperature for five hours after which it was diluted with dichloromethane and washed with aqueous saturated sodium bicarbonate and brine. The organic layer was dried with anhydrous magnesium sulfate, filtered and concentrated. The crude material was purified by silica gel chromatography in ethyl acetate/dichloromethane to obtain compound 72 as a white crystalline solid (0.68 g, y=89%). MS (m/z): 507.1 (M+1)⁺505.1 (M−1)⁻ UPLC=1.51 min (2.5 min method).

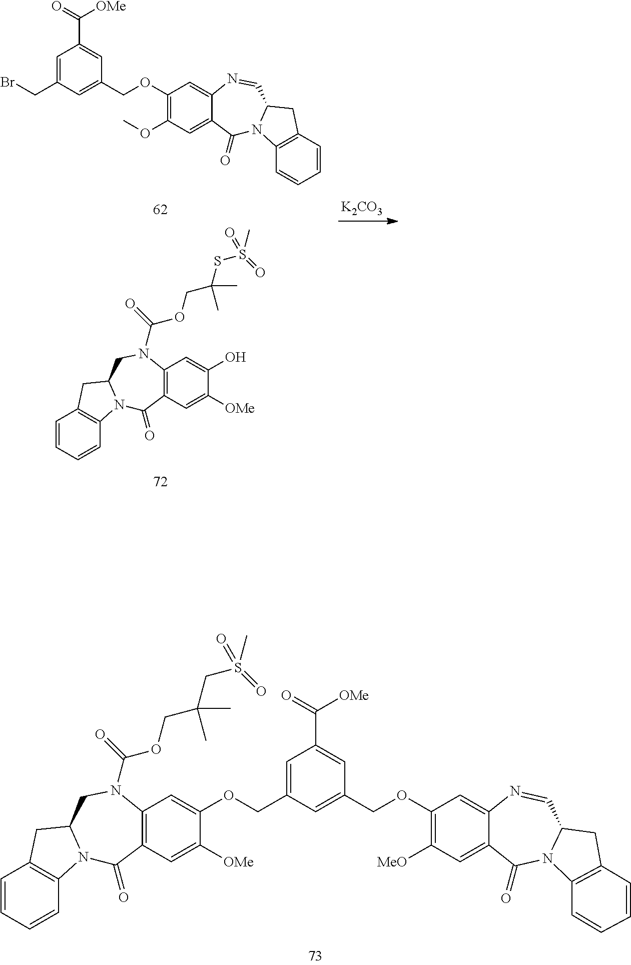

Compound 62 (69.6 mg, 0.130 mmol) and compound 72 (59 mg, 0.108 mmol) were dissolved in anhydrous dimethylacetamide (1083 μl). Potassium carbonate (29.9 mg, 0.217 mmol) was added and the reaction was stirred overnight at room temperature. The reaction was slurried with water and filtered. The resulting solid was dissolved in dichloromethane with a little methanol, dried over anhydrous magnesium sulfate and concentrated to get 0.114 mg of compound 73 (70% pure, y=76%) as a crude yellow solid. Pure material was obtained by RP-HPLC (C18, deionized water/acetonitrile). MS (m/z): 961.7 (M+1)⁺. UPLC=1.85 min (2.5 min method).

### Example 9. Synthesis of Compound 75

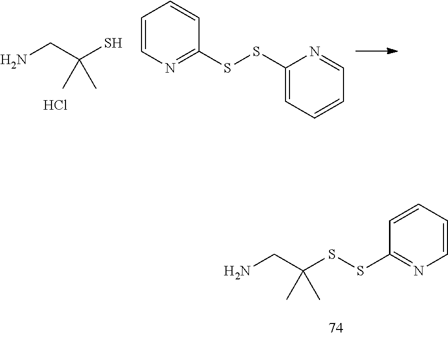

Dimethylcysteamine HCl (500 mg, 3.53 mmol) was dissolved in methanol (11.765 mL). Added Aldrithiol (1166 mg, 5.29 mmol) and stirred yellow solution overnight. LCMS indicated product formation at 0.394 minutes. Added triethylamine (0.492 mL, 3.53 mmol) and stirred for 5 minutes and then the reaction mixture was concentrated. The crude residue was purified by silica gel chromatography (dichloromethane/methanol) to give compound 74 as an off white sticky solid (700 mg, 93%).

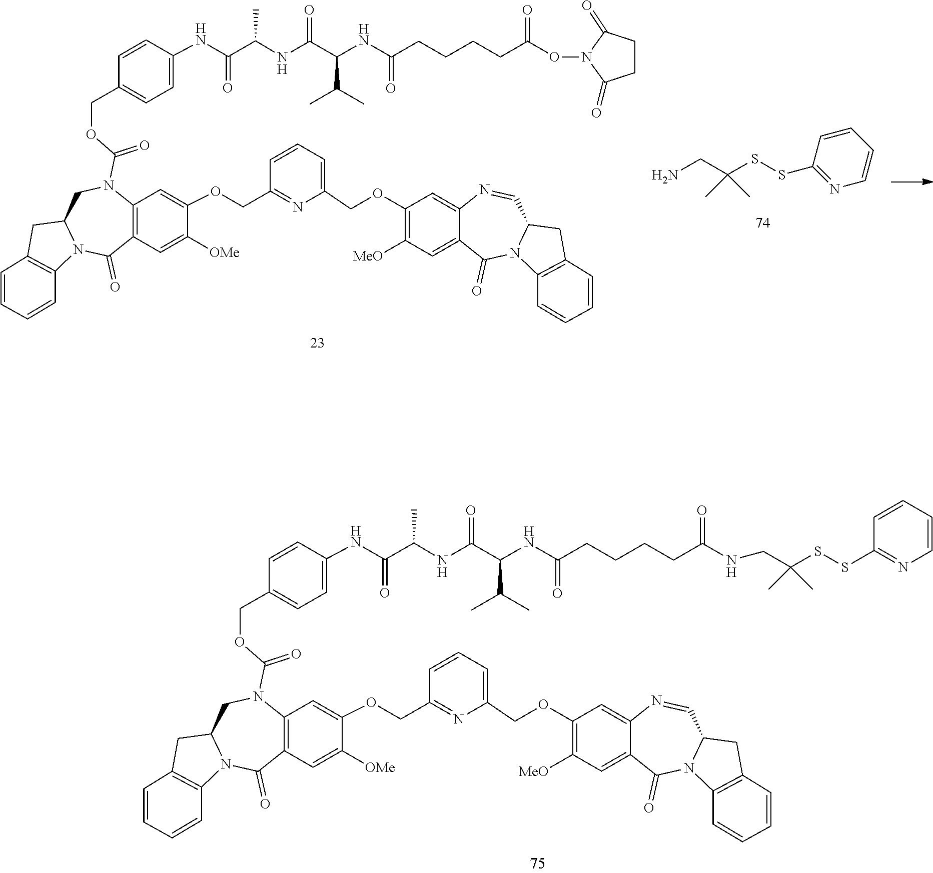 [SMILES: COc1cc2c(cc1OCc1cccc(COc3cc4c(cc3OC)C(=O)N3c5ccccc5C[C@H]3CN4C(=O)OCc3ccc(NC(=O)[C@H](C)NC(=O)[C@@H](NC(=O)CCCCC(=O)ON4C(=O)CCC4=O)C(C)C)cc3)n1)N=C[C@@H]1Cc3ccccc3N1C2=O ; CC(C)(CN)SSc1ccccn1 ; COc1cc2c(cc1OCc1cccc(COc3cc4c(cc3OC)C(=O)N3c5ccccc5C[C@H]3CN4C(=O)OCc3ccc(NC(=O)[C@H](C)NC(=O)[C@@H](NC(=O)CCCCC(=O)NCC(C)(C)SSc4ccccn4)C(C)C)cc3)n1)N=C[C@@H]1Cc3ccccc3N1C2=O]

Compound 23 (10 mg, 8.08 μmol) was dissolved in anhydrous dichloromethane (0.25 ml). Compound 74 (2.077 mg, 9.69 μmol) and then DIPEA (2.81 μl, 0.016 mmol) were added. The reaction stirred for 1 hour and was concentrated to dryness. The crude material was dissolved in acetonitrile/H2O with 2 drops formic acid and purified by RP-HPLC (C18, deionized water/acetonitrile). The desired fractions were frozen and lyophilized to give compound 75 (8 mg, y=74%). MS (m/z): 1338.5 (M+1)⁺. UPLC=1.88 min (2.5 min method).

### Example 10. Synthesis of Compound 79

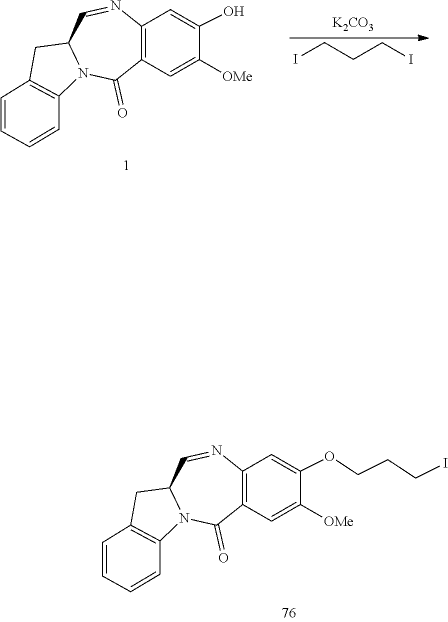 [SMILES: COc1cc2c(cc1O)N=C[C@@H]1Cc3ccccc3N1C2=O ; "K2CO3" ; ICCCI ; COc1cc2c(cc1OCCCI)N=C[C@@H]1Cc3ccccc3N1C2=O]

Compound 1 (250 mg, 0.807 mmol) was dissolved in anhydrous dimethylformamide (4035 μl). 1,3-diiodopropane (1399 μl, 12.10 mmol) and potassium carbonate (335 mg, 2.421 mmol) were added. The reaction stirred overnight at room temperature. It was diluted with ethyl acetate and water. The organic was separated and washed with water three times. It was dried, concentrated, and purified by silica gel chromatography (hexanes/ethyl acetate) to provide compound 76 (215 mg, 57%).

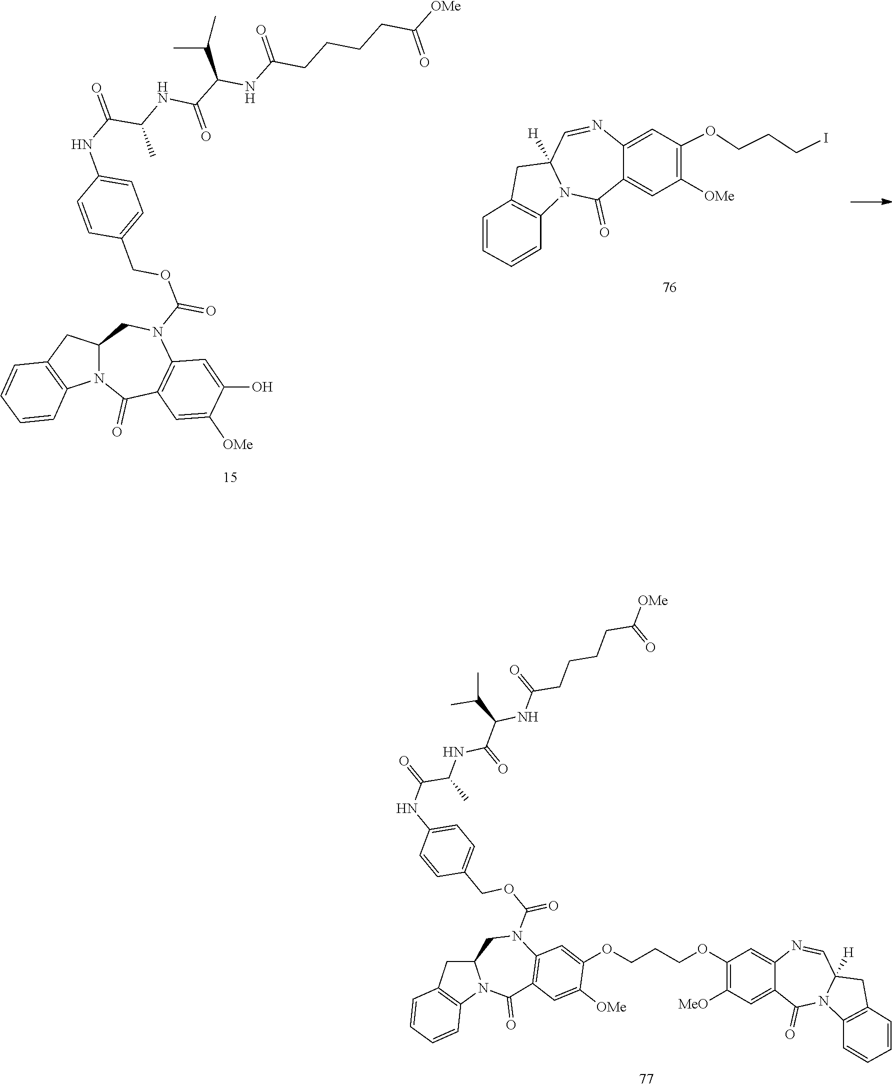 [SMILES: COC(=O)CCCCC(=O)N[C@@H](C(=O)N[C@H](C)C(=O)Nc1ccc(COC(=O)N2C[C@@H]3Cc4ccccc4N3C(=O)c3cc(OC)c(O)cc32)cc1)C(C)C ; COc1cc2c(cc1OCCCI)N=C[C@@H]1Cc3ccccc3N1C2=O ; COC(=O)CCCCC(=O)N[C@@H](C(=O)N[C@H](C)C(=O)Nc1ccc(COC(=O)N2C[C@@H]3Cc4ccccc4N3C(=O)c3cc(OC)c(OCCCOc4cc5c(cc4OC)C(=O)N4c6ccccc6C[C@@H]4C=N5)cc32)cc1)C(C)C]

Compounds 15 and 76 were reacted similarly as Compound 16. The crude material was purified by silica gel chromatography (dichloromethane/methanol) to give compound 77 (54.3 mg, 85% pure, y=30%). MS (m/z): 1093.0 (M+1)⁺. UPLC=1.83 min (2.5 min method).

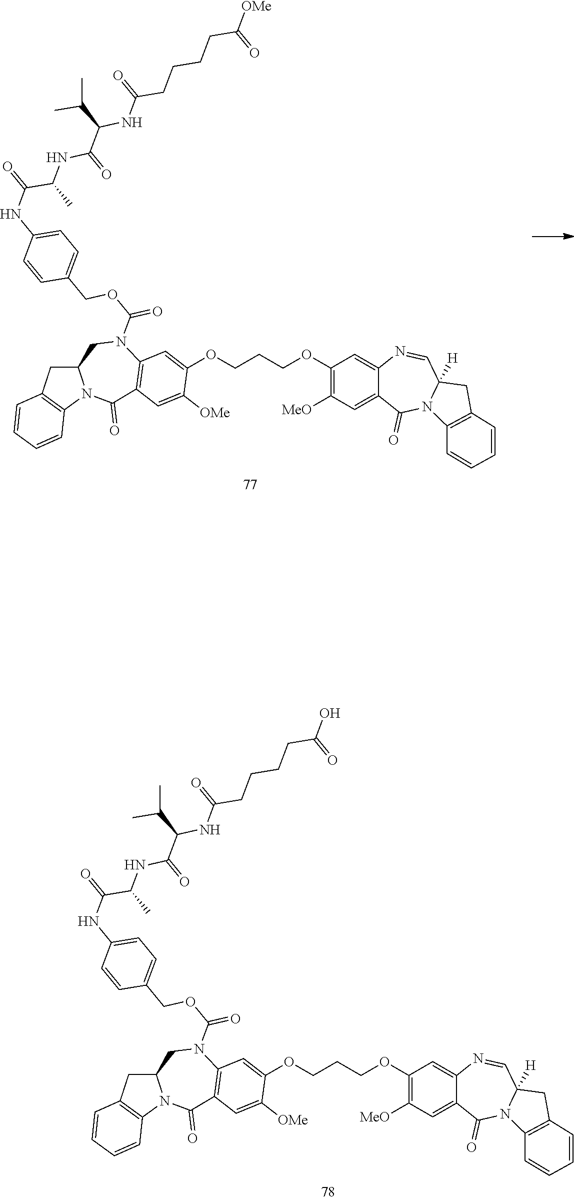 [SMILES: COC(=O)CCCCC(=O)N[C@@H](C(=O)N[C@H](C)C(=O)Nc1ccc(COC(=O)N2C[C@@H]3Cc4ccccc4N3C(=O)c3cc(OC)c(OCCCOc4cc5c(cc4OC)C(=O)N4c6ccccc6C[C@@H]4C=N5)cc32)cc1)C(C)C ; COc1cc2c(cc1OCCCOc1cc3c(cc1OC)C(=O)N1c4ccccc4C[C@H]1CN3C(=O)OCc1ccc(NC(=O)[C@@H](C)NC(=O)[C@H](NC(=O)CCCCC(=O)O)C(C)C)cc1)N=C[C@H]1Cc3ccccc3N1C2=O]

Compound 78 was prepared similarly as Compound 17. The crude material was used directly in the next reaction, assuming 100% yield. MS (m/z): 1078.8 (M+1)⁺. UPLC=1.74 (2.5 min method).

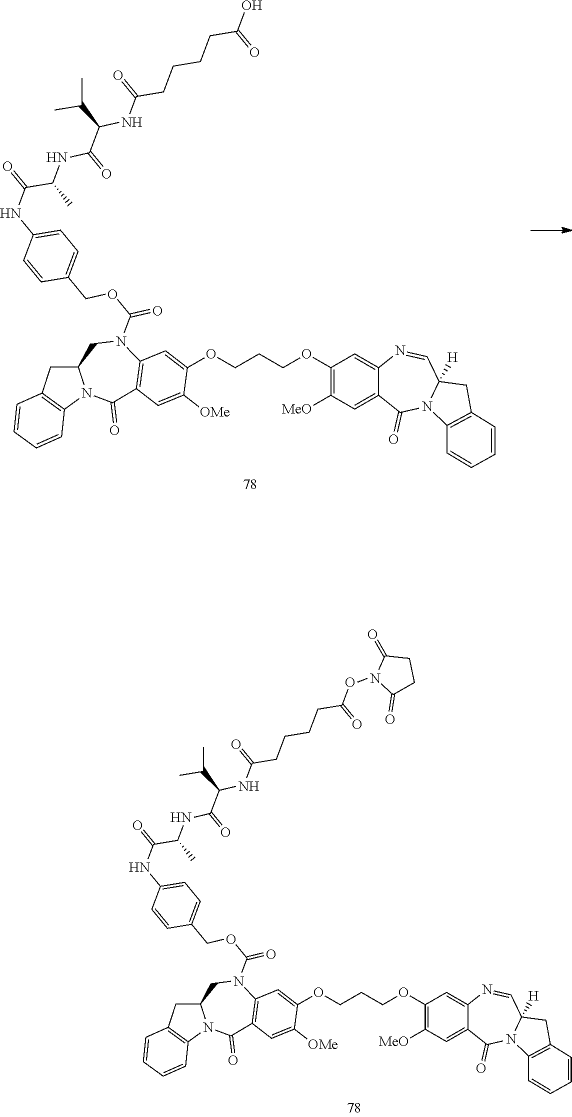 [SMILES: COc1cc2c(cc1OCCCOc1cc3c(cc1OC)C(=O)N1c4ccccc4C[C@H]1CN3C(=O)OCc1ccc(NC(=O)[C@@H](C)NC(=O)[C@H](NC(=O)CCCCC(=O)O)C(C)C)cc1)N=C[C@H]1Cc3ccccc3N1C2=O ; COc1cc2c(cc1OCCCOc1cc3c(cc1OC)C(=O)N1c4ccccc4C[C@H]1CN3C(=O)OCc1ccc(NC(=O)[C@@H](C)NC(=O)[C@H](NC(=O)CCCCC(=O)ON3C(=O)CCC3=O)C(C)C)cc1)N=C[C@H]1Cc3ccccc3N1C2=O]

Compound 79 was prepared similarly as Compound 18. The crude material was purified via RPHPLC (C18 column, water/acetonitrile) to give the final compound 79 (38 mg, y=65%). MS (m/z): 1175.8 (M+1)⁺. UPLC=1.80 min (2.5 min method).

### Example 11. Synthesis of Compound 83

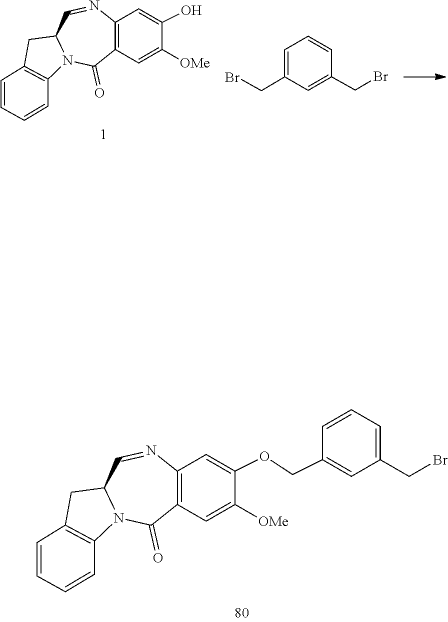 [SMILES: COc1cc2c(cc1O)N=C[C@@H]1Cc3ccccc3N1C2=O ; BrCc1cccc(CBr)c1 ; COc1cc2c(cc1OCc1cccc(CBr)c1)N=C[C@@H]1Cc3ccccc3N1C2=O]

Compound 1 (250 mg, 0.807 mmol) was dissolved in anhydrous dimethylformamide (4035 μl). 1,3-bis(bromomethyl)benzene (1704 mg, 6.46 mmol) and potassium carbonate (335 mg, 2.421 mmol) were added. The reaction stirred overnight at room temperature. It was crashed out with water and filtered. The solid was purified by silica gel chromatography to remove excess starting materials and compound 80 was used immediately in the next reaction. MS (m/z): 477.4 (M+1)⁺. UPLC=1.77 (2.5 min method).

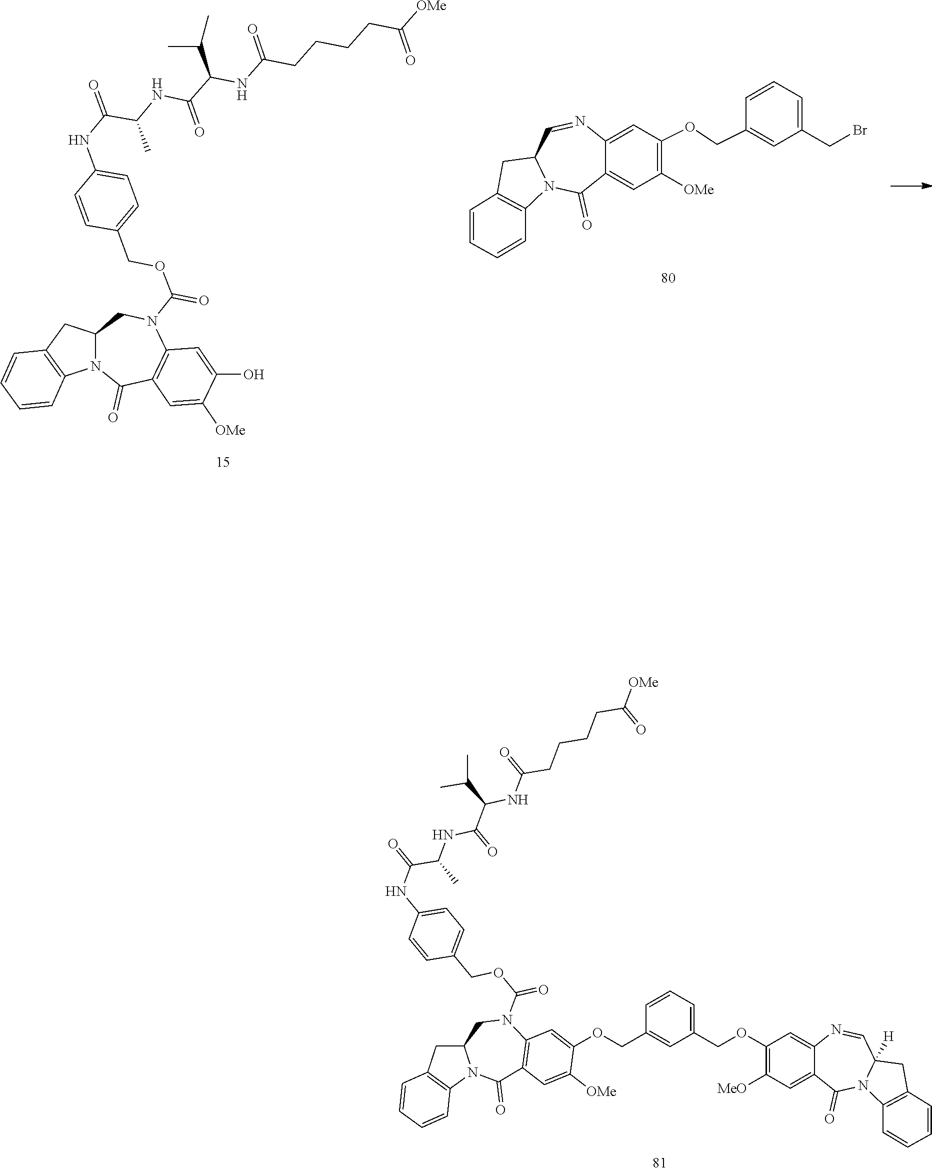 [SMILES: COC(=O)CCCCC(=O)N[C@@H](C(=O)N[C@H](C)C(=O)Nc1ccc(COC(=O)N2C[C@@H]3Cc4ccccc4N3C(=O)c3cc(OC)c(O)cc32)cc1)C(C)C ; COc1cc2c(cc1OCc1cccc(CBr)c1)N=C[C@@H]1Cc3ccccc3N1C2=O ; COC(=O)CCCCC(=O)N[C@@H](C(=O)N[C@H](C)C(=O)Nc1ccc(COC(=O)N2C[C@@H]3Cc4ccccc4N3C(=O)c3cc(OC)c(OCc4cccc(COc5cc6c(cc5OC)C(=O)N5c7ccccc7C[C@@H]5C=N6)c4)cc32)cc1)C(C)C]

CoCCompound 80 (64.6 mg, 0.135 mmol) was dissolved in anhydrous dimethylacetamide (3260 μl). Potassium carbonate (45.1 mg, 0.326 mmol) and compound 15 (124 mg, 0.163 mmol) were sequentially added and the reaction proceeded to completion (12-15 h) at room temperature under argon. The reaction was precipitated with water and filtered. The collected solid was dissolved in dichloromethane, transferred to a separatory funnel, washed with water, brine, dried over anhydrous sodium sulfate and concentrated in vacuo. It was purified via silica gel chromatography (dichloromethane/methanol) to give compound 81 (93.7 mg, y=50%). MS (m/z): 1154.8 (M+1)⁺. UPLC=1.92 (2.5 min method).

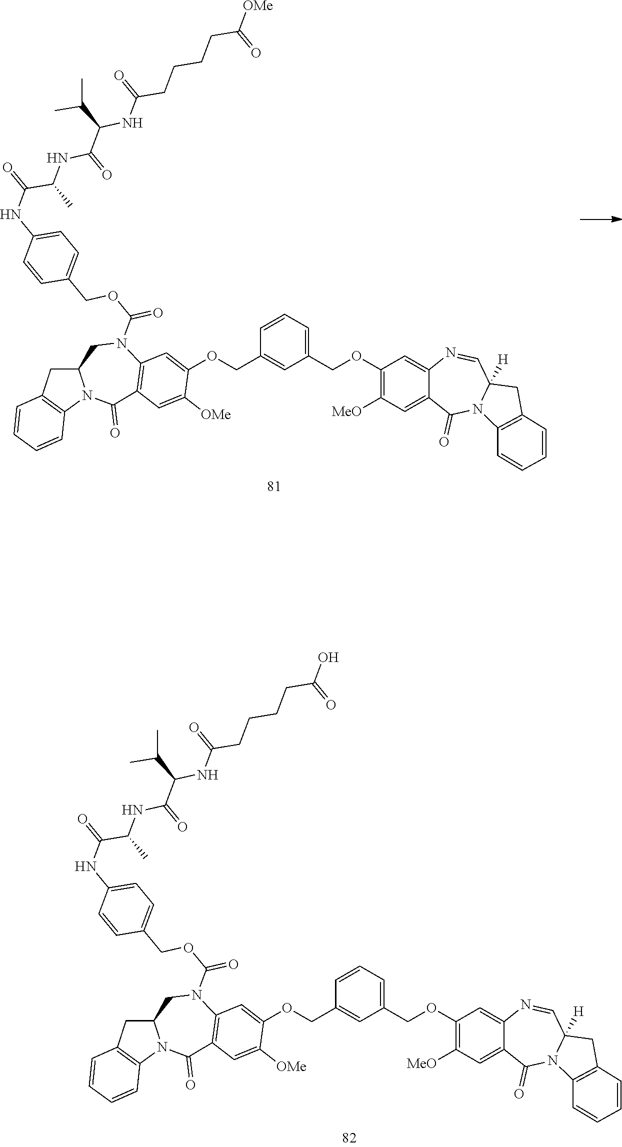 [SMILES: COC(=O)CCCCC(=O)N[C@@H](C(=O)N[C@H](C)C(=O)Nc1ccc(COC(=O)N2C[C@@H]3Cc4ccccc4N3C(=O)c3cc(OC)c(OCc4cccc(COc5cc6c(cc5OC)C(=O)N5c7ccccc7C[C@@H]5C=N6)c4)cc32)cc1)C(C)C ; COc1cc2c(cc1OCc1cccc(COc3cc4c(cc3OC)C(=O)N3c5ccccc5C[C@H]3CN4C(=O)OCc3ccc(NC(=O)[C@@H](C)NC(=O)[C@H](NC(=O)CCCCC(=O)O)C(C)C)cc3)c1)N=C[C@H]1Cc3ccccc3N1C2=O]

Compound 82 was prepared similarly as Compound 17. The crude material was used without further purification. Assumed 100% yield. MS (m/z): 1140.9 (M+1)⁺. UPLC=1.85 (2.5 min method).

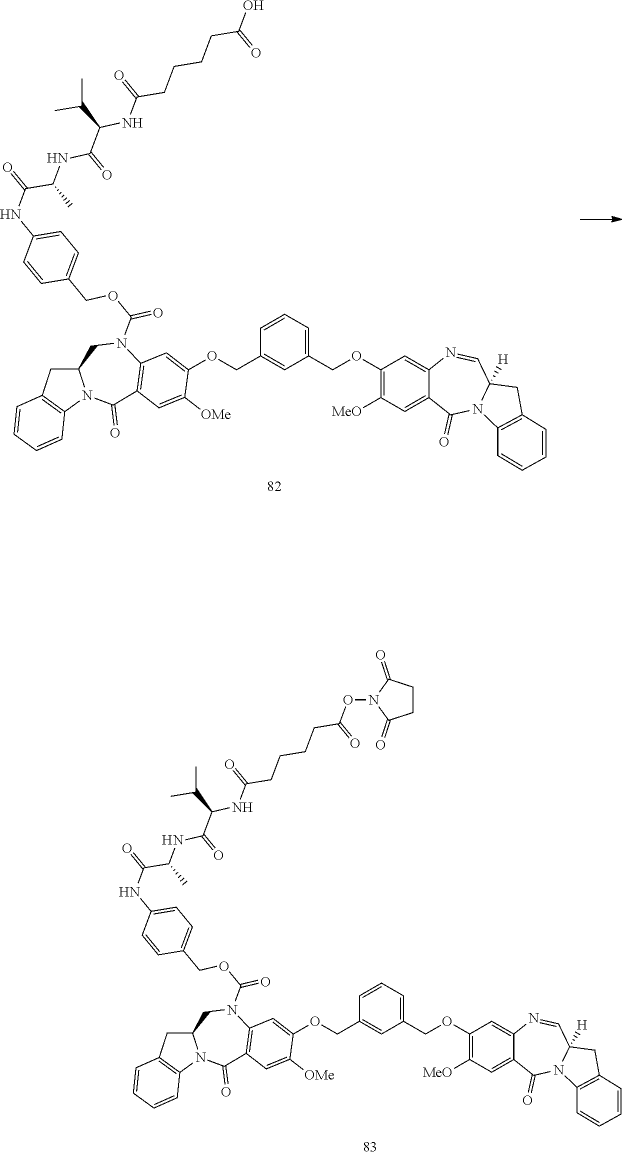 [SMILES: COc1cc2c(cc1OCc1cccc(COc3cc4c(cc3OC)C(=O)N3c5ccccc5C[C@H]3CN4C(=O)OCc3ccc(NC(=O)[C@@H](C)NC(=O)[C@H](NC(=O)CCCCC(=O)O)C(C)C)cc3)c1)N=C[C@H]1Cc3ccccc3N1C2=O ; COc1cc2c(cc1OCc1cccc(COc3cc4c(cc3OC)C(=O)N3c5ccccc5C[C@H]3CN4C(=O)OCc3ccc(NC(=O)[C@@H](C)NC(=O)[C@H](NC(=O)CCCCC(=O)ON4C(=O)CCC4=O)C(C)C)cc3)c1)N=C[C@H]1Cc3ccccc3N1C2=O]

Compound 83 was prepared similarly as Compound 18. The crude material was purified via RPHPLC (C18 column, water/acetonitrile) to give the final compound 83 (13.8 mg, y14%). MS (m/z): 1237.7 (M+1)⁺. UPLC 1.92 min (2.5 mm method).

### Example 12. Synthesis of Compound 95

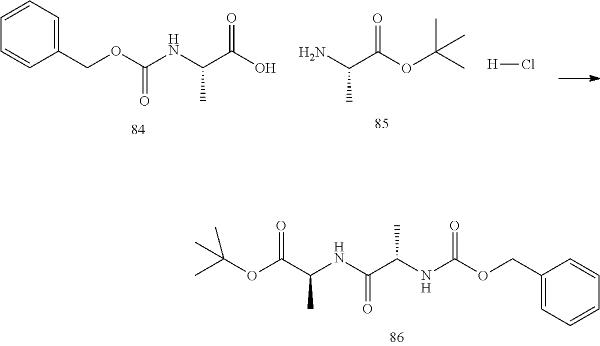

(S)-2-(((benzyloxy)carbonyl)amino)propanoic acid (5 g, 22.40 mmol) and (S)-tert-butyl 2-aminopropanoate hydrochloride (4.48 g, 24.64 mmol) were dissolved in anhydrous N,N-Dimethylformamide (44.8 ml). 3-(3-Dimethylaminopropyl)-1-ethyl-carbodiimide hydrochloride (4.72 g, 24.64 mmol), 1-Hydroxybenzotriazole hydrate (3.43 g, 22.40 mmol), and diisopropylethylamine (9.75 ml, 56.0 mmol) were added. The reaction stirred under argon, at room temperature, overnight. The reaction mixture was diluted with dichloromethane and then washed with saturated ammonium chloride, saturated sodium bicarbonate, water, and brine. The organic was dried over sodium sulfate and concentrated. The crude oil was purified via silica gel chromatography (hexanes/ethyl acetate) to yield compound 86 (6.7 g, y=85%)¹H NMR (400 Hz, CDCl₃): δ 7.38-7.31 (m, 5H), 6.53-6.42 (m, 1H), 5.42-5.33 (m, 1H), 5.14 (s, 2H), 4.48-4.41 (m, 1H), 4.32-4.20 (m, 1H), 1.49 (s, 9H), 11.42 (d, J=6.8 Hz, 3H), 1.38 (d, J=7.2 Hz, 3H).

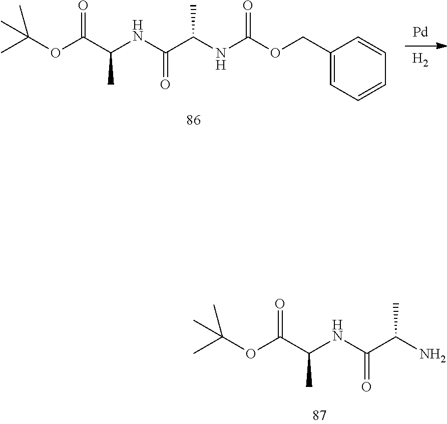

Compound 86 (6.7 g, 19.12 mmol) was dissolved in methanol (60.7 ml) and water (3.03 ml). The solution was purged with argon for five minutes. Palladium on carbon (wet, 10%) (1.017 g, 0.956 mmol) was added slowly. The reaction was stirred overnight under an atmosphere of hydrogen. The solution was filtered through celite, rinsed with methanol and concentrated. It was azeotroped with methanol and acetonitrile and the resulting oil was placed directly on the high vacuum to give compound 87 (4.02 g, y=97%) which was used directly in the next step. H NMR (400 Hz, CDCl₃): δ 7.78-7.63 (m, 1H), 4.49-4.42 (m, 1H), 3.55-3.50 (m, 1H), 1.73 (s, 2H), 1.48 (s, 9H), 1.39 (d, J=7.2 Hz, 3H), 1.36 (d, J=6.8 Hz, 3H).

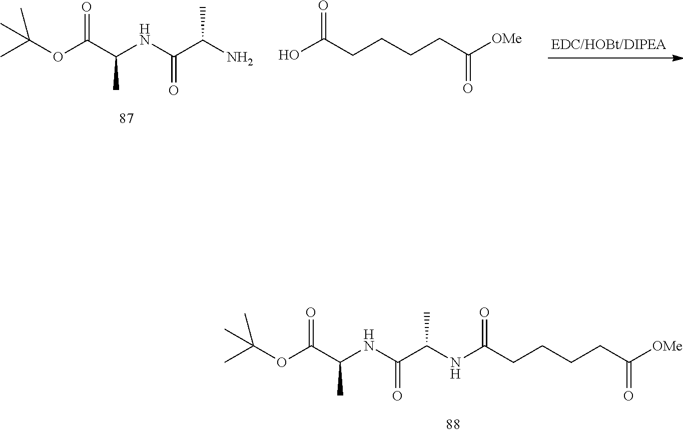 [SMILES: C[C@H](N)C(=O)N[C@@H](C)C(=O)OC(C)(C)C ; COC(=O)CCCCC(=O)O ; "EDC/HOBt/DIPEA" ; COC(=O)CCCCC(=O)N[C@@H](C)C(=O)N[C@@H](C)C(=O)OC(C)(C)C]

Compound 87 (4.02 g, 18.59 mmol) and monomethyladipate (3.03 ml, 20.45 mmol) were dissolved in anhydrous N,N-Dimethylformamide (62.0 ml). 3-(3-Dimethylaminopropyl)-1-ethyl-carbodiimide hydrochloride (3.92 g, 20.45 mmol) and 1-Hydroxybenzotriazole hydrate ((2.85 g, 18.59 mmol) and diispopropylethylamine (6.49 ml, 37.2 mmol) were added. The mixture was stirred overnight at room temperature. The reaction was diluted with dichloromethane/methanol (150 mL, 5:1) and washed with saturated ammonium chloride, saturated sodium bicarbonate, and brine. It was dried over sodium sulfate, filtered and stripped. The compound was azeotroped with acetonitrile (5×), then pumped on the high vacuum at 35° C. to give compound 88 (6.66 g, y=100%). The crude material was taken onto next step without purification. ¹H NMR (400 Hz, CDCl₃): δ 6.75 (d, J=6.8 Hz, 1H), 6.44 (d, J=6.8 Hz, 1H), 4.52-4.44 (m, 1H), 4.43-4.36 (m, 1H), 3.65 (s, 3H), 2.35-2.29 (m, 2H), 2.25-2.18 (m, 2H), 1.71-1.60 (m, 4H), 1.45 (s, 9H), 1.36 (t, J=6 Hz, 12.8 Hz, 6H)

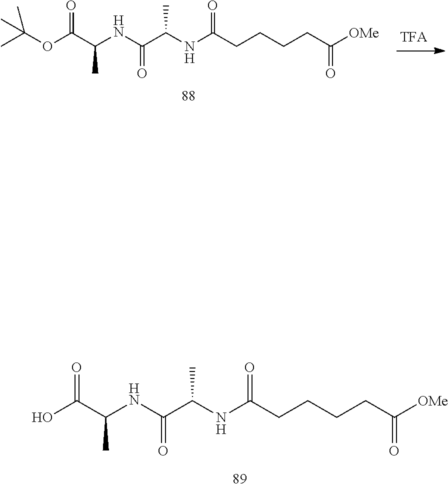 [SMILES: COC(=O)CCCCC(=O)N[C@@H](C)C(=O)N[C@@H](C)C(=O)OC(C)(C)C ; COC(=O)CCCCC(=O)N[C@@H](C)C(=O)N[C@@H](C)C(=O)O ; "TFA"]

Compound 88 (5.91 g, 16.5 mmol) was stirred in trifluoroacetic acid (28.6 ml, 372 mmol) and deionized water (1.5 ml) at room temperature for three hours. It was rotovaped with acetonitrile and placed on high vacuum until the solvents were removed to give crude compound 89 as a sticky solid (5.88 g y=105%). ¹H NMR (400 Hz, CDCl₃): δ 7.21 (d, J=6.8 Hz, 1H), 6.81 (d, J=7.6 Hz, 1H), 4.69-4.60 (m, 1H), 4.59-4.51 (m, 1H), 3.69 (s, 3H), 2.40-2.33 (m, 2H), 2.31-2.24 (m, 2H), 1.72-1.63 (m, 4H), 1.51-1.45 (m, 3H), 1.42-1.37 (m, 3H)

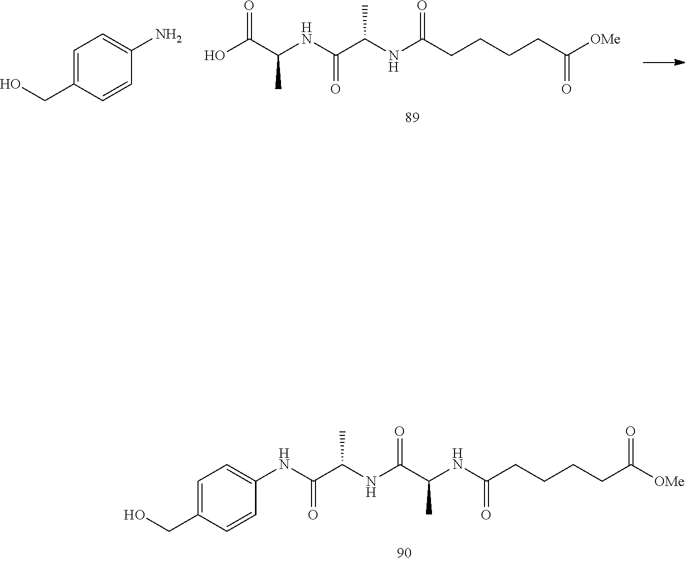 [SMILES: Nc1ccc(CO)cc1 ; COC(=O)CCCCC(=O)N[C@@H](C)C(=O)N[C@@H](C)C(=O)O ; COC(=O)CCCCC(=O)N[C@@H](C)C(=O)N[C@@H](C)C(=O)Nc1ccc(CO)cc1]

Compound 90 was prepared similarly as Compound 13. The crude material was slurried with ethyl acetate and filtered. (800 mg, y=81%). LCMS=3.1 min (8 min method).

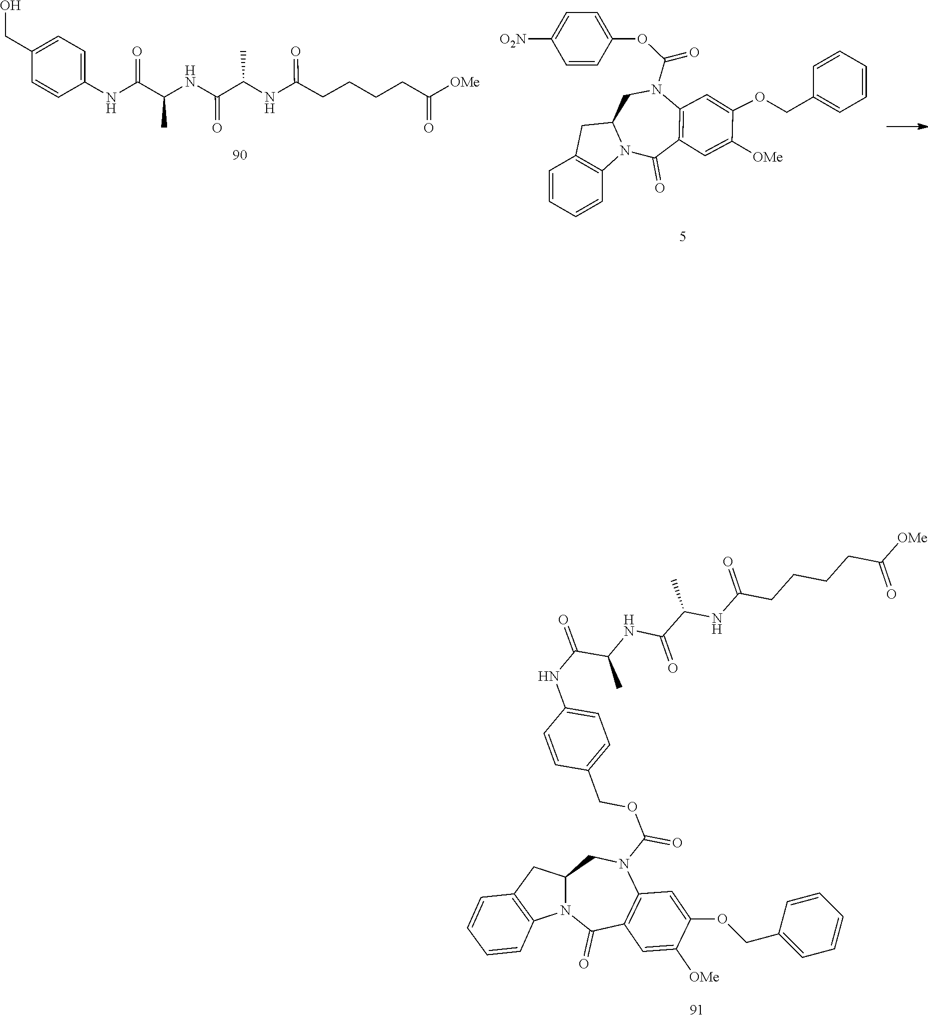 [SMILES: COC(=O)CCCCC(=O)N[C@@H](C)C(=O)N[C@@H](C)C(=O)Nc1ccc(CO)cc1 ; COc1cc2c(cc1OCc1ccccc1)N(C(=O)Oc1ccc([N+](=O)[O-])cc1)C[C@@H]1Cc3ccccc3N1C2=O ; COC(=O)CCCCC(=O)N[C@@H](C)C(=O)N[C@@H](C)C(=O)Nc1ccc(COC(=O)N2C[C@@H]3Cc4ccccc4N3C(=O)c3cc(OC)c(OCc4ccccc4)cc32)cc1]

Compound 91 was prepared similarly as Compound 14. The crude material was purified by silica gel chromatography (dichloromethane/methanol) (52 mg, y=17.5%). MS (m/z): 820.6 (M+1)⁺. MS (m/z): 818.7 (M−1)−. UPLC=5.5 min (10 min method).

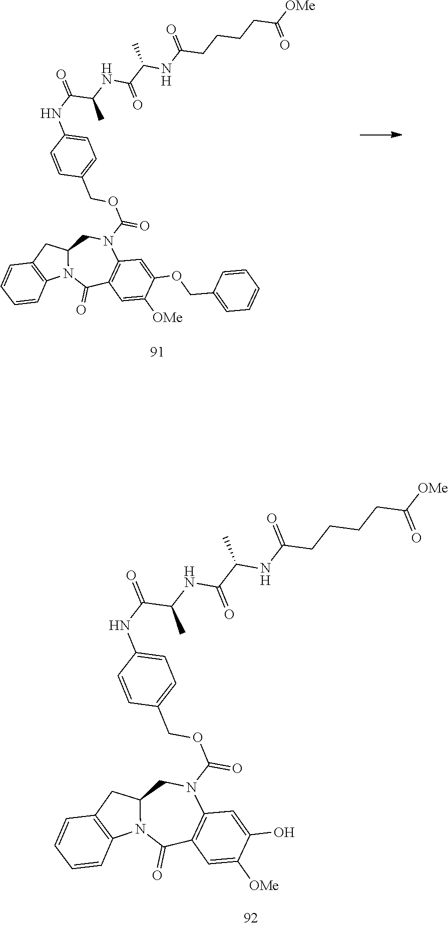 [SMILES: COC(=O)CCCCC(=O)N[C@@H](C)C(=O)N[C@@H](C)C(=O)Nc1ccc(COC(=O)N2C[C@@H]3Cc4ccccc4N3C(=O)c3cc(OC)c(OCc4ccccc4)cc32)cc1 ; COC(=O)CCCCC(=O)N[C@@H](C)C(=O)N[C@@H](C)C(=O)Nc1ccc(COC(=O)N2C[C@@H]3Cc4ccccc4N3C(=O)c3cc(OC)c(O)cc32)cc1]

To a solution of compound 91 (10 mg, 0.012 mmol) in ethanol (47.7 μl, 0.817 mmol) was added cyclohexa-1,4-diene (1.745 μl, 0.018 mmol), (dimethyl-13-sulfanyl)-11-oxidane (0.077 μl, 1.085 μmol) followed by palladium (1.953 mg, 0.018 mmol). The reaction was stirred at 45C overnight at which point there was ˜50% conversion. Additional Pd/Alox, DMSO, cyclohexadiene, were added using similar ratio and quantity as precedent. The reaction was completed after 4 h. The Pd was then filtered on celite and the crude solution evaporated. The filtered product was used in the next step without further purification.

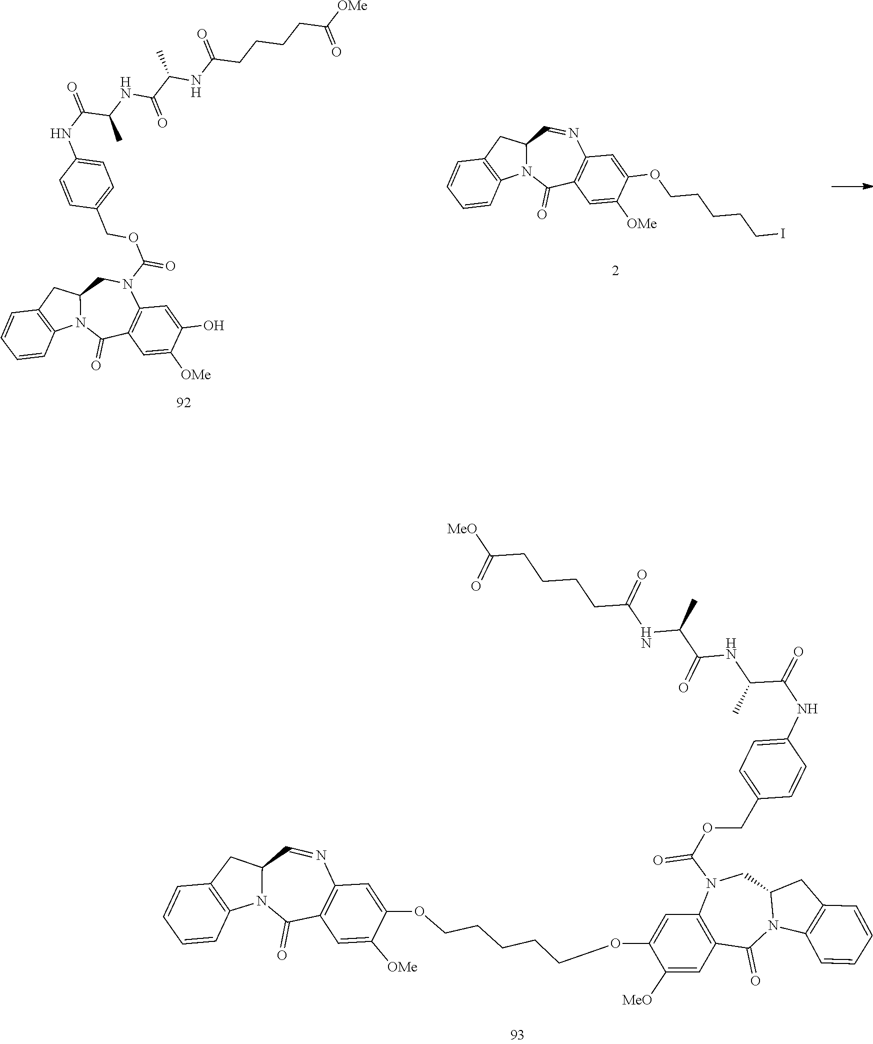 [SMILES: COC(=O)CCCCC(=O)N[C@@H](C)C(=O)N[C@@H](C)C(=O)Nc1ccc(COC(=O)N2C[C@@H]3Cc4ccccc4N3C(=O)c3cc(OC)c(O)cc32)cc1 ; COc1cc2c(cc1OCCCCCI)N=C[C@@H]1Cc3ccccc3N1C2=O ; COC(=O)CCCCC(=O)N[C@@H](C)C(=O)N[C@@H](C)C(=O)Nc1ccc(COC(=O)N2C[C@@H]3Cc4ccccc4N3C(=O)c3cc(OC)c(OCCCCCOc4cc5c(cc4OC)C(=O)N4c6ccccc6C[C@H]4C=N5)cc32)cc1]

Compound 92 and Compound 2 were reacted similarly as in the synthesis of Compound 16. Compound 93 was used without purification in the next reaction. (52 mg, y=67%). MS (m/z): 1093.2 (M+1)⁺. UPLC=5.78 min (10 min method).

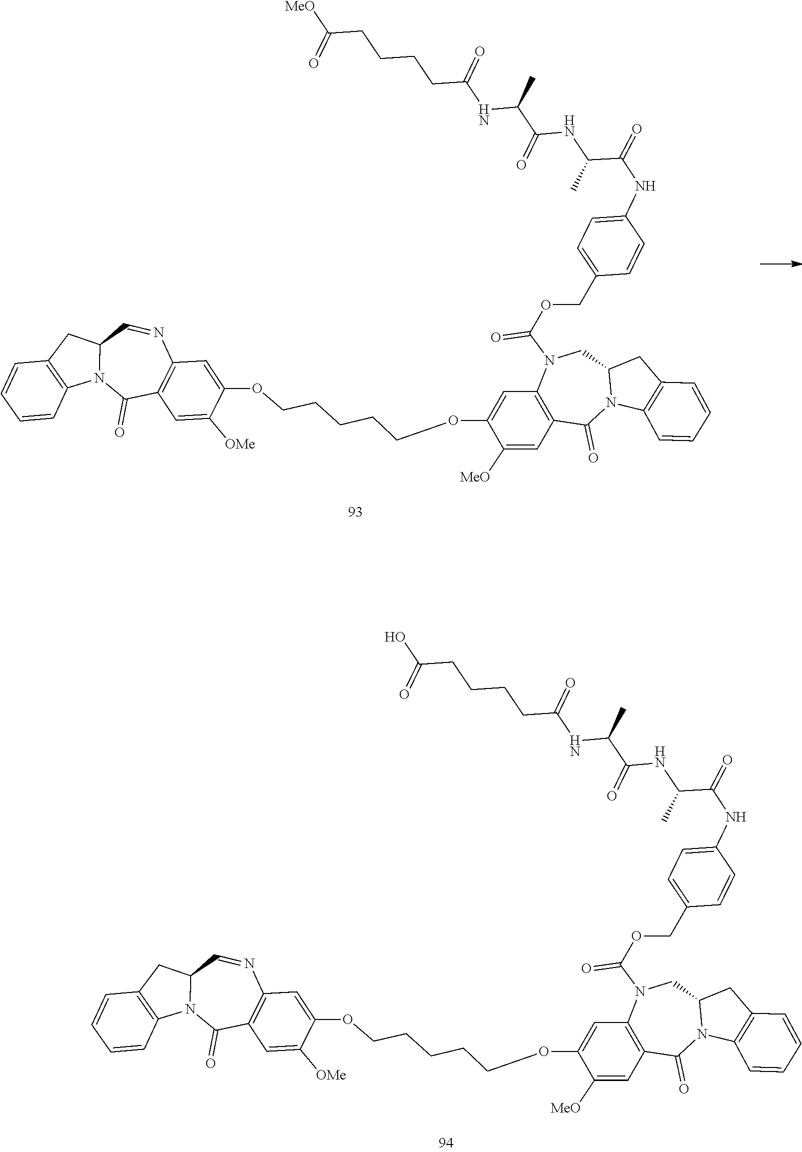 [SMILES: COC(=O)CCCCC(=O)N[C@@H](C)C(=O)N[C@@H](C)C(=O)Nc1ccc(COC(=O)N2C[C@@H]3Cc4ccccc4N3C(=O)c3cc(OC)c(OCCCCCOc4cc5c(cc4OC)C(=O)N4c6ccccc6C[C@H]4C=N5)cc32)cc1 ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@H](C)NC(=O)CCCCC(=O)O)cc1)N=C[C@@H]1Cc3ccccc3N1C2=O]

Compound 94 was prepared similarly as Compound 17. The crude material was used without further purification (33 mg, y=96%). MS (m/z): 1079.2 (M+1)⁺. UPLC=1.76 (2.5 min method).

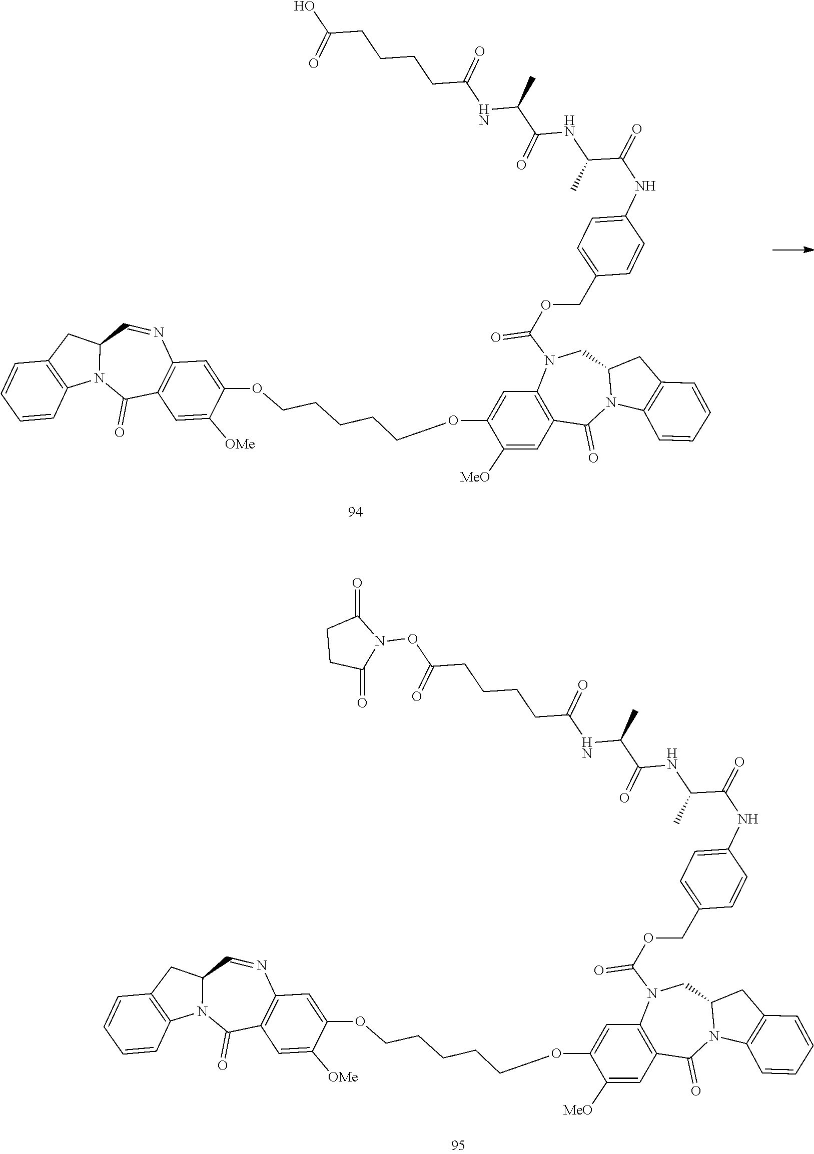 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@H](C)NC(=O)CCCCC(=O)O)cc1)N=C[C@@H]1Cc3ccccc3N1C2=O ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@H](C)NC(=O)CCCCC(=O)ON3C(=O)CCC3=O)cc1)N=C[C@@H]1Cc3ccccc3N1C2=O]

To a solution of compound 94 (33 mg, 0.031 mmol) in anhydrous dichloromethane (1 ml) was added N-ethyl-N-isopropylpropan-2-amine (8.23 μl, 0.046 mmol) followed by bis(2,5-dioxopyrrolidin-1-yl) carbonate (10.19 mg, 0.040 mmol) at room temperature. The reaction was stirred until the starting material was consumed. It was then quenched with water, the layers were separated, and the aqueous layer extracted once with dichloromethane. The combined organic layers were washed with brine, dried over magnesium sulfate and filtered. The solvent was removed and crude material purified by RPHPLC (water/acetonitrile) to give final compound 95 (12 mg, y=33%). MS (m/z): 1176.4 (M+1)⁺. UPLC=1.84 (2.5 min method).

### Example 13. Synthesis of Compound 99

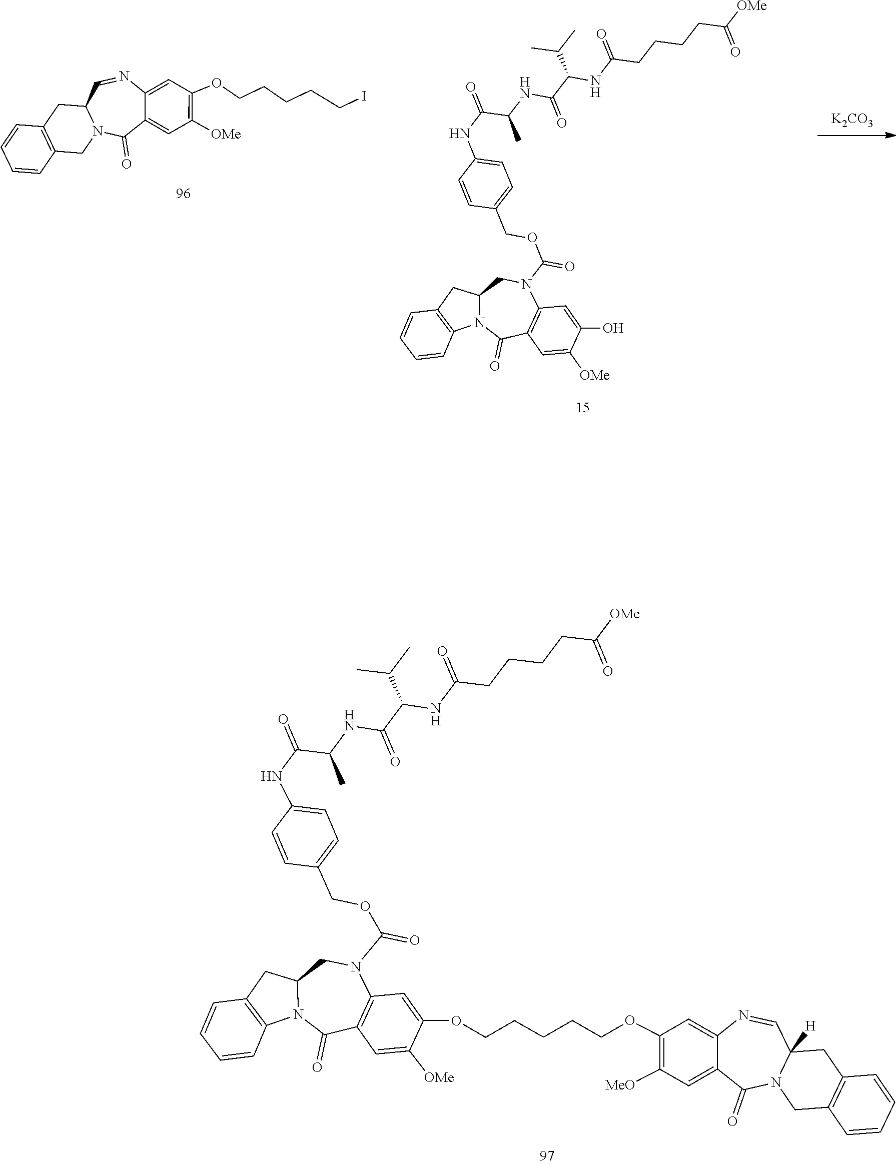 [SMILES: COc1cc2c(cc1OCCCCCI)N=C[C@@H]1Cc3ccccc3CN1C2=O ; COC(=O)CCCCC(=O)N[C@H](C(=O)N[C@@H](C)C(=O)Nc1ccc(COC(=O)N2C[C@@H]3Cc4ccccc4N3C(=O)c3cc(OC)c(O)cc32)cc1)C(C)C ; "K2CO3" ; COC(=O)CCCCC(=O)N[C@H](C(=O)N[C@@H](C)C(=O)Nc1ccc(COC(=O)N2C[C@@H]3Cc4ccccc4N3C(=O)c3cc(OC)c(OCCCCCOc4cc5c(cc4OC)C(=O)N4Cc6ccccc6C[C@H]4C=N5)cc32)cc1)C(C)C]

To a solution of (S)-3-((5-iodopentyl)oxy)-2-methoxy-7,12-dihydrobenzo[5,6][1,4]diazepino [1,2-b]isoquinolin-14(6aH)-one (51.1 mg, 0.101 mmol) and 4-((S)-2-((S)-2-(6-methoxy-6-oxohexanamido)-3-methylbutanamido)propanamido)benzyl (S)-9-hydroxy-8-methoxy-6-oxo-12a,13-dihydro-6H-benzo[5,6][1,4]diazepino[1,2-a]indole-11(12H)-carboxylate (64 mg, 0.084 mmol) in anhydrous N,N-Dimethylacetamide (845 μl) was added potassium carbonate (23.34 mg, 0.169 mmol) and the reaction was stirred under nitrogen at ambient temperature for 18 hours. Deionized water (10 mL) was added to the reaction and the resulting white solid was filtered, then re-dissolved in dichloromethane, transferred to a separatory funnel, and washed with water. The organic layer was dried over anhydrous magnesium sulfate, filtered, and concentrated in vacuo. The crude solid was purified via silica gel chromotography in methanol/dichloromethane to obtain compound 97 (80 mg, y=84%) as a white solid. MS (m/z): 1135.1 (M+1)⁺. UPLC=5.91 min (10 min method).

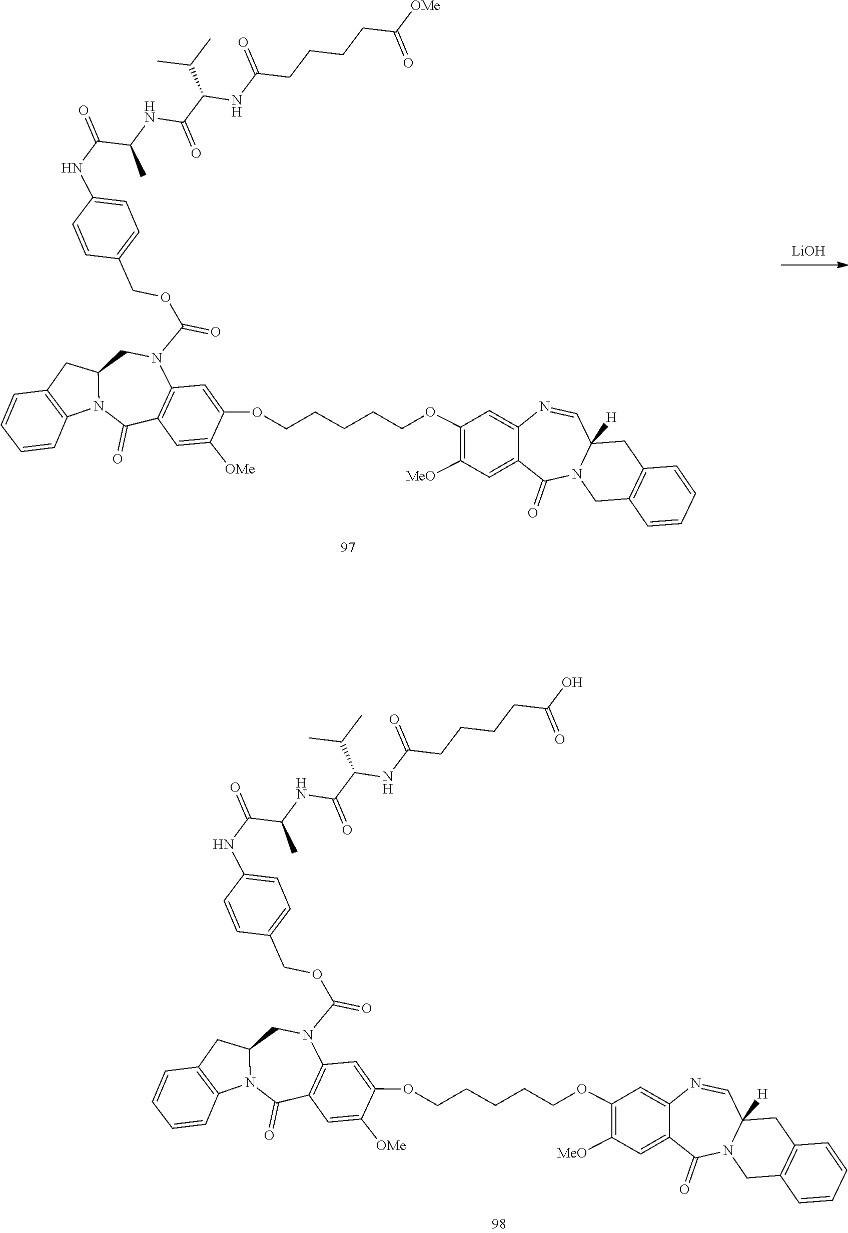 [SMILES: COC(=O)CCCCC(=O)N[C@H](C(=O)N[C@@H](C)C(=O)Nc1ccc(COC(=O)N2C[C@@H]3Cc4ccccc4N3C(=O)c3cc(OC)c(OCCCCCOc4cc5c(cc4OC)C(=O)N4Cc6ccccc6C[C@H]4C=N5)cc32)cc1)C(C)C ; "LiOH" ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)O)C(C)C)cc1)N=C[C@@H]1Cc3ccccc3CN1C2=O]

To a cooled solution (0° C.) of 4-((S)-2-((S)-2-(6-methoxy-6-oxohexanamido)-3-methylbutanamido)propanamido)benzyl (S)-8-methoxy-9-((5-(((S)-2-methoxy-14-oxo-6a,7,12,14-tetrahydrobenzo[5,6] [1,4]diazepino[1,2-b]isoquinolin-3-yl)oxy)pentyl)oxy)-6-oxo-12a,13-dihydro-6H-benzo[5,6][1,4]diazepino[1,2-a]indole-11(12H)-carboxylate (62 mg, 0.055 mmol) in anhydrous Tetrahydrofuran (2.0 ml) and deionized water (683 μl) was added lithium hydroxide (3.93 mg, 0.164 mmol). The reaction stirred at ambient temperature under nitrogen for 90 min after which the mixture was diluted with 20% methanol/dichloromethane (10 ml) and deionized water (5 ml). The mixture was acidified to pH=3 with aqueous hydrochloric acid (0.5M, 1 mL) and extracted with 20% methanol/dichloromethane (2×20 mL). The organic layer was washed with deionized water, dried with anhydrous magnesium sulfate, filtered through celite and concentrated. The crude material was purified by silica gel chromatography in methanol/dichloromethane to yield DP (36 mg, y=58%). MS (m/z): 1121.3 (M+1)⁺. UPLC=1.66 min (2.5 min method).

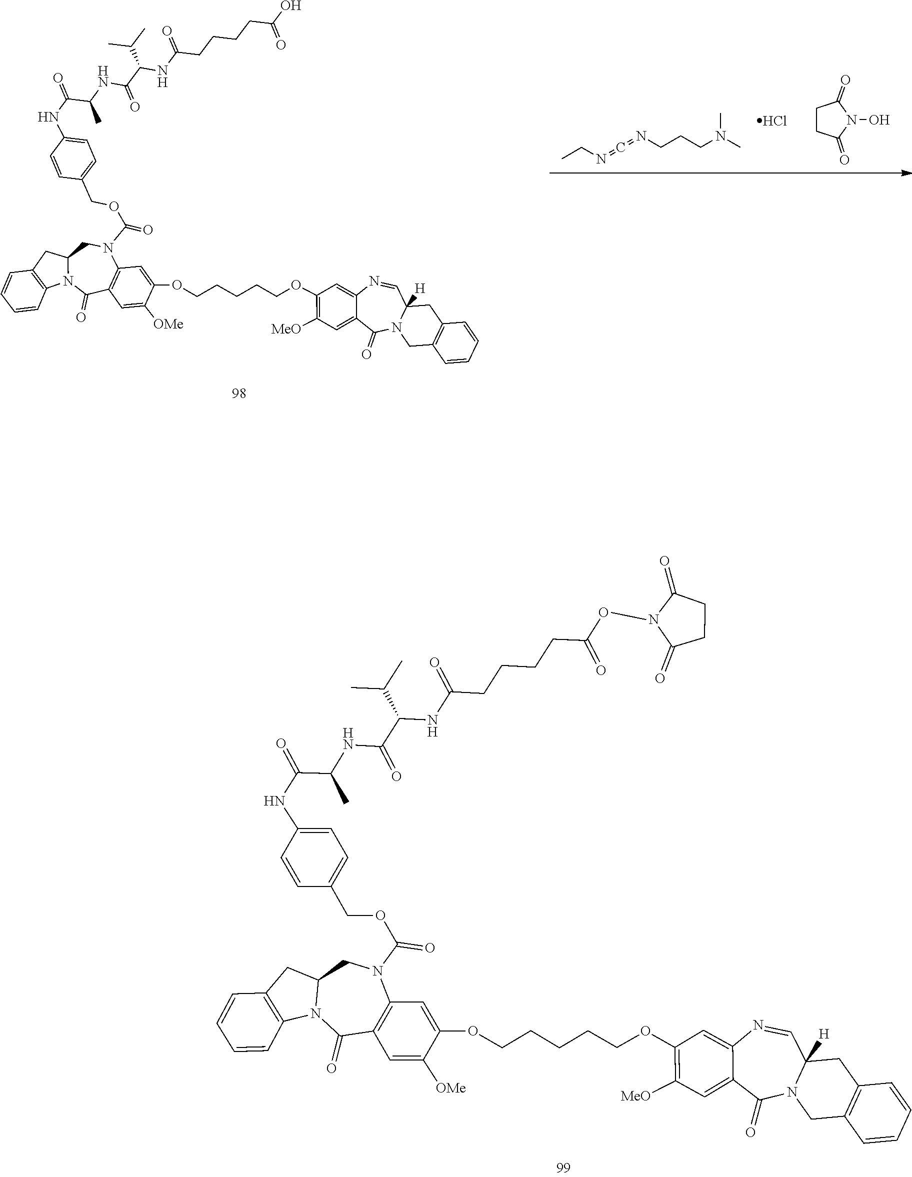 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)O)C(C)C)cc1)N=C[C@@H]1Cc3ccccc3CN1C2=O ; CCN=C=NCCCN(C)C ; Cl ; O=C1CCC(=O)N1O ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)ON3C(=O)CCC3=O)C(C)C)cc1)N=C[C@@H]1Cc3ccccc3CN1C2=O]

To a solution of compound 98 (36 mg, 0.032 mmol) and N-hydroxy succinimide (11.10 mg, 0.096 mmol) in anhydrous dichloromethane (321 μl) was added 1-ethyl-3-(3-dimethylaminopropyl) carbodiimide hydrochloride (30.8 mg, 0.161 mmol). The reaction was stirred for two hours under nitrogen at ambient temperature upon which it was diluted with dichloromethane and washed with water. The organic layer was dried with anhydrous magnesium sulfate, filtered and concentrated in vacuo. The crude product was purified by RP-HPLC (Kromasil C18, acetonitrile/Deionized water, 50-65% over 30 min) and fractions containing product were frozen and lyophilized to yield compound 99 (18 mg, y=46%) as a white solid. MS (m/z): 1218.1 (M+1)⁺. UPLC=1.75 min (2.5 min method).

### Example 14. Synthesis of Compound 106

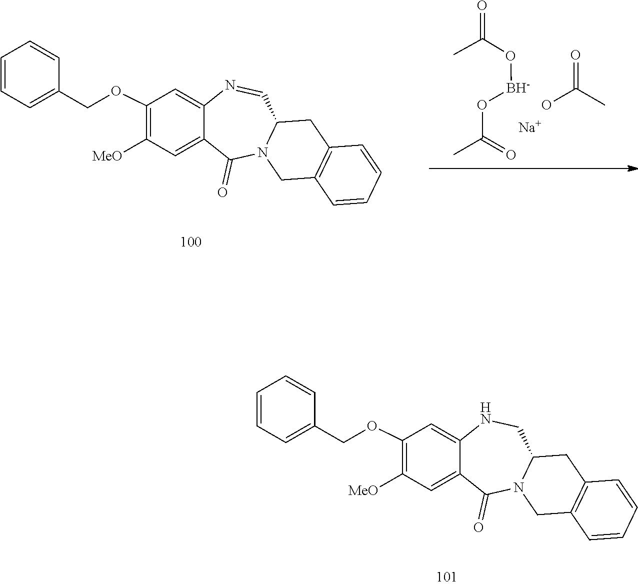

To a suspension of (S)-3-(benzyloxy)-2-methoxy-7,12-dihydrobenzo[5,6][1,4]diazepino [1,2-b]isoquinolin-14(6aH)-one (1.1 g, 2.76 mmol) in anhydrous 1,2-Dichloroethane (18.40 ml) was added sodium triacetoxyborohydride (1.755 g, 8.28 mmol) and the mixture was stirred for 20 hours at ambient temperature. The mixture was quenched with saturated aqueous ammonium chloride solution and extracted with dichloromethane. The organic extracts were washed with brine solution, dried with anhydrous magnesium sulfate, filtered and concentrated to obtain crude (S)-3-hydroxy-2-methoxy-6,6a,7,12-tetrahydrobenzo[5,6][1,4]diazepino[1,2-b]isoquinolin-14(5H)-one as a light yellow solid, which was used for next reaction without purification (1.1 g, y=99%). MS (m/z): 401.6 (M+1)⁺. UPLC=1.69 min (2.5 min method).

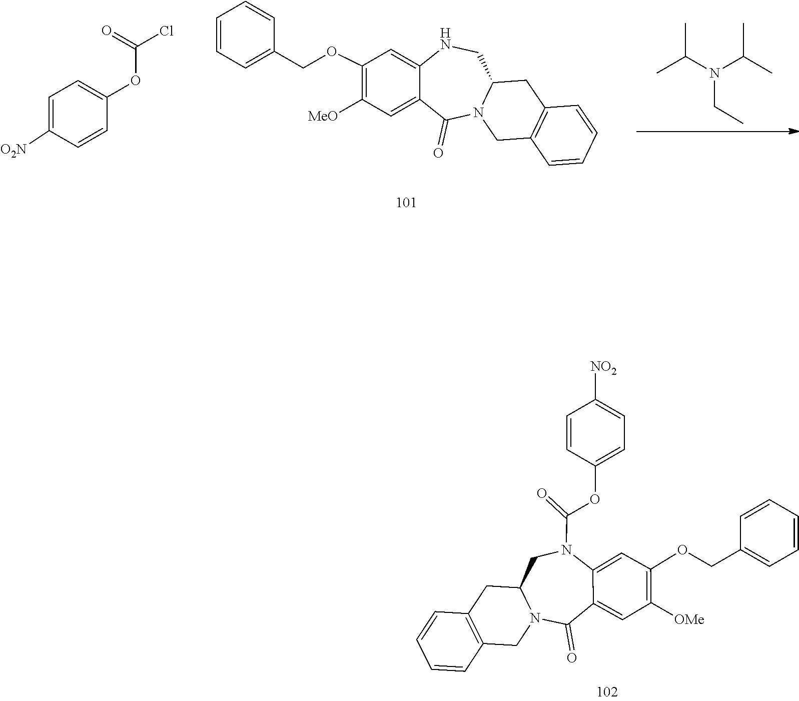

To a suspension of (S)-3-(benzyloxy)-2-methoxy-6,6a,7,12-tetrahydrobenzo[5,6][1,4]diazepino[1,2-b]isoquinolin-14(5H)-one (1.1 g, 2.75 mmol) in anhydrous dichloromethane (12.21 ml) was added N-ethyl-N-isopropylpropan-2-amine (0.591 ml, 3.30 mmol). Then a solution of 4-nitrophenyl carbonochloridate (0.634 g, 3.02 mmol) in anhydrous dichloromethane (6.10 ml) was added. The white slurry became clear yellow and continued stirring at ambient temperature for 20 hours. The reaction was diluted with dichloromethane and water and the layers were separated. The organic layer was washed with water (50 ml), saturated aqueous sodium bicarbonate solution (50 ml), brine solution (50 mL), dried over anhydrous magnesium sulfate, filtered and concentrated. The resulting solid was purified on ISCO (ethyl acetate/hexanes, 40 g silica column) to yield compound 102 (1.2 g, 77% yield). MS (m/z): 566.5 (M+1)⁺. UPLC=1.94 min (2.5 min method).

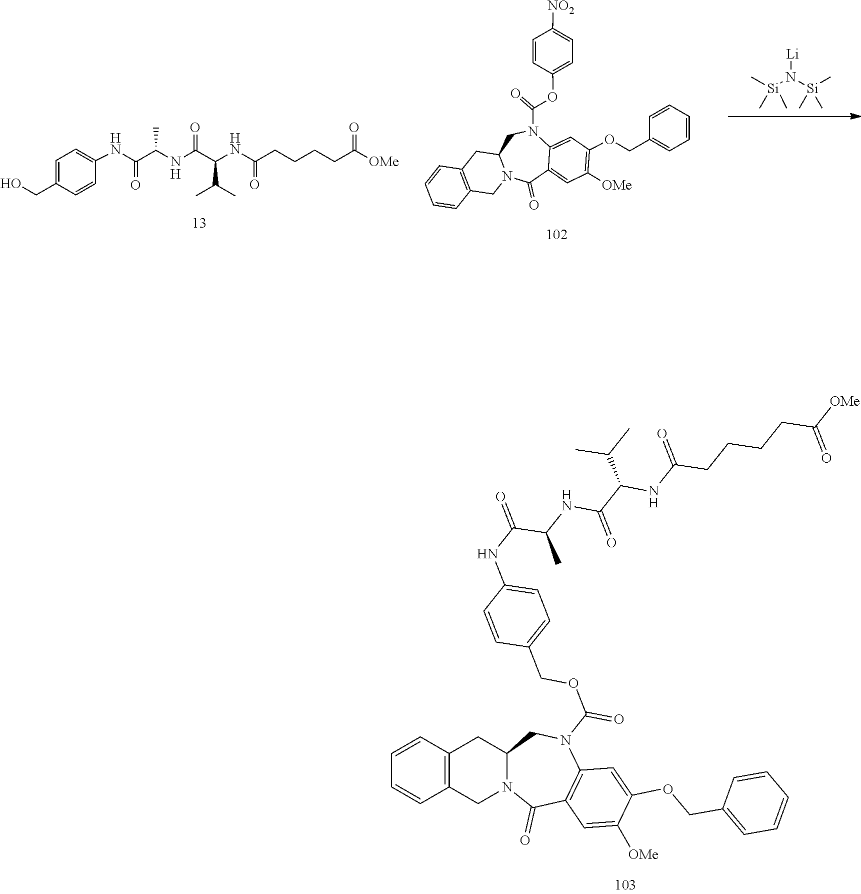

To a solution of methyl 6-(((S)-1-(((S)-1-((4-(hydroxymethyl)phenyl)amino)-1-oxopropan-2-yl)amino)-3-methyl-1-oxobutan-2-yl)amino)-6-oxohexanoate (410 mg, 0.941 mmol) in anhydrous tetrahydrofuran (1.78 ml) and anhydrous N,N-dimethylacetamide (3.56 ml) was added lithium bis(trimethylsilyl)amide (1M in tetrahydrofuran, 0.94 ml, 0.941 mmol) at 0° C. The clear yellow reaction was stirred for 15 min before 4-nitrophenyl (S)-3-(benzyloxy)-2-methoxy-14-oxo-6,6a,7,12-tetrahydrobenzo[5,6][1,4]diazepino[1,2-b]isoquinoline-5(14H)-carboxylate (444 mg, 0.785 mmol) in anhydrous tetrahydrofuran (1.78 ml) was added.

The reaction mixture was stirred for 18 hours under nitrogen from 0° C. to ambient temperature, upon which it was cooled in an ice bath and quenched with saturated aqueous ammonium chloride solution. The mixture was extracted with dichloromethane and the combined organic layers were washed with water and brine, then dried with anhydrous magnesium sulfate, filtered and concentrated on high vacuum to remove N,N-dimethylacetamide. The crude yellow oil was purified by silca gel chromatography in methanol/dichloromethane to yield 4-((S)-2-((S)-2-(6-methoxy-6-oxohexanamido)-3-methylbutanamido)propanamido)benzyl (S)-3-(benzyloxy)-2-methoxy-14-oxo-6,6a,7,12-tetrahydrobenzo[5,6][1,4]diazepino[1,2-b]isoquinoline-5(14H)-carboxylate (81 mg, y=27%). MS (m/z): 862.8 (M+1)⁺860.5 (M−1)−. UPLC=1.82 min (2.5 min method).

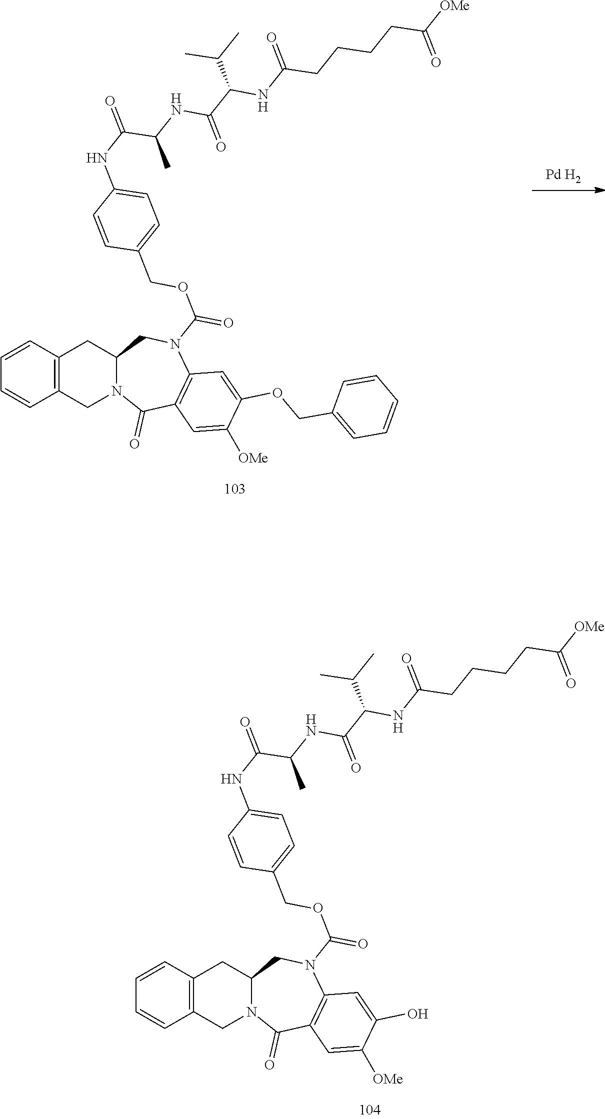 [SMILES: COC(=O)CCCCC(=O)N[C@H](C(=O)N[C@@H](C)C(=O)Nc1ccc(COC(=O)N2C[C@@H]3Cc4ccccc4CN3C(=O)c3cc(OC)c(OCc4ccccc4)cc32)cc1)C(C)C ; "Pd H2" ; COC(=O)CCCCC(=O)N[C@H](C(=O)N[C@@H](C)C(=O)Nc1ccc(COC(=O)N2C[C@@H]3Cc4ccccc4CN3C(=O)c3cc(OC)c(O)cc32)cc1)C(C)C]

A solution of 4-((S)-2-((S)-2-(6-methoxy-6-oxohexanamido)-3-methylbutanamido)propanamido)benzyl (S)-3-(benzyloxy)-2-methoxy-14-oxo-6,6a,7,12-tetrahydrobenzo[5,6][1,4]diazepino[1,2-b]isoquinoline-5(14H)-carboxylate (197 mg, 0.229 mmol) in anhydrous methanol (2.28 ml) was degassed with nitrogen and palladium on carbon (10%, 24.32 mg, 0.229 mmol) was added. The mixture was evacuated and stirred at ambient temperature under hydrogen balloon (1 atm) for two hours. The reaction mixture was filtered rinsing with 20% methanol/dichloromethane. The filtrate was concentrated and purified by silica gel chromatography in methanol/dichloromethane to obtain compound 104 as a bright white solid (111 mg, y=63%). MS (m/z): 772.8 (M+1)⁺, 770.7 (M−1)⁻. UPLC=1.52 min (2.5 min method).

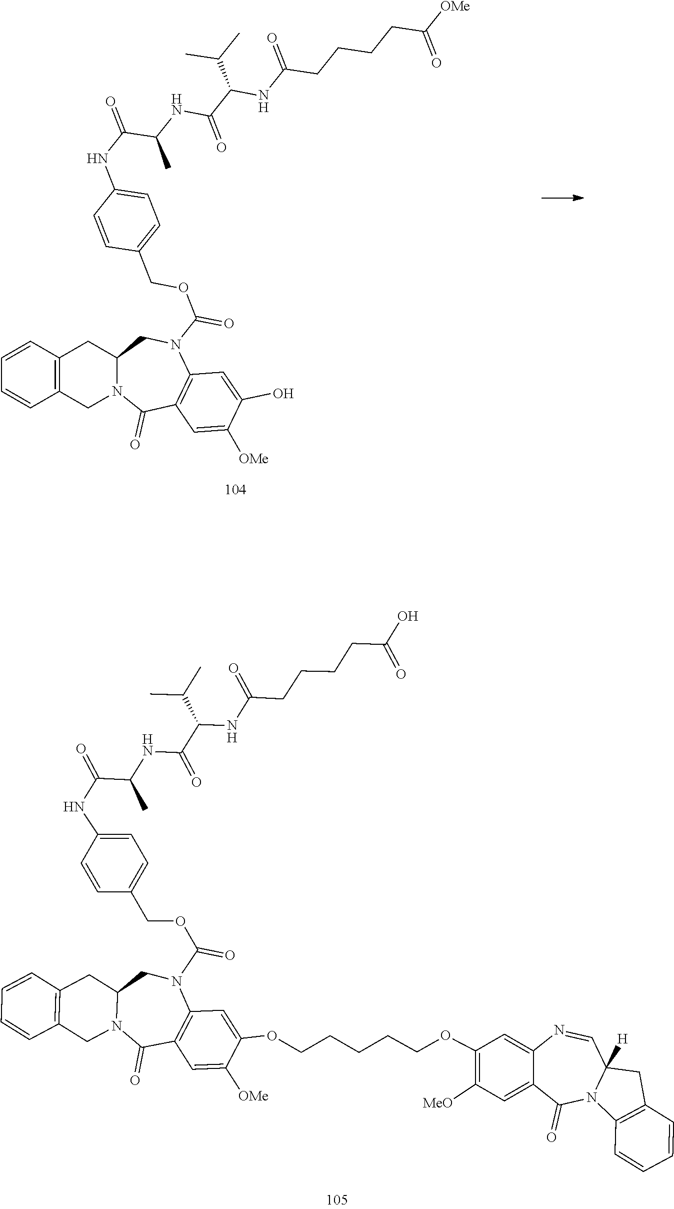 [SMILES: COC(=O)CCCCC(=O)N[C@H](C(=O)N[C@@H](C)C(=O)Nc1ccc(COC(=O)N2C[C@@H]3Cc4ccccc4CN3C(=O)c3cc(OC)c(O)cc32)cc1)C(C)C ; COc1cc2c(cc1OCCCCCOc1cc3c(cc1OC)C(=O)N1Cc4ccccc4C[C@H]1CN3C(=O)OCc1ccc(NC(=O)[C@H](C)NC(=O)[C@@H](NC(=O)CCCCC(=O)O)C(C)C)cc1)N=C[C@@H]1Cc3ccccc3N1C2=O]

Compound 105 was prepared from compound 104 in a similar approach as the conversion of compound 15 to compound 17 over two steps.

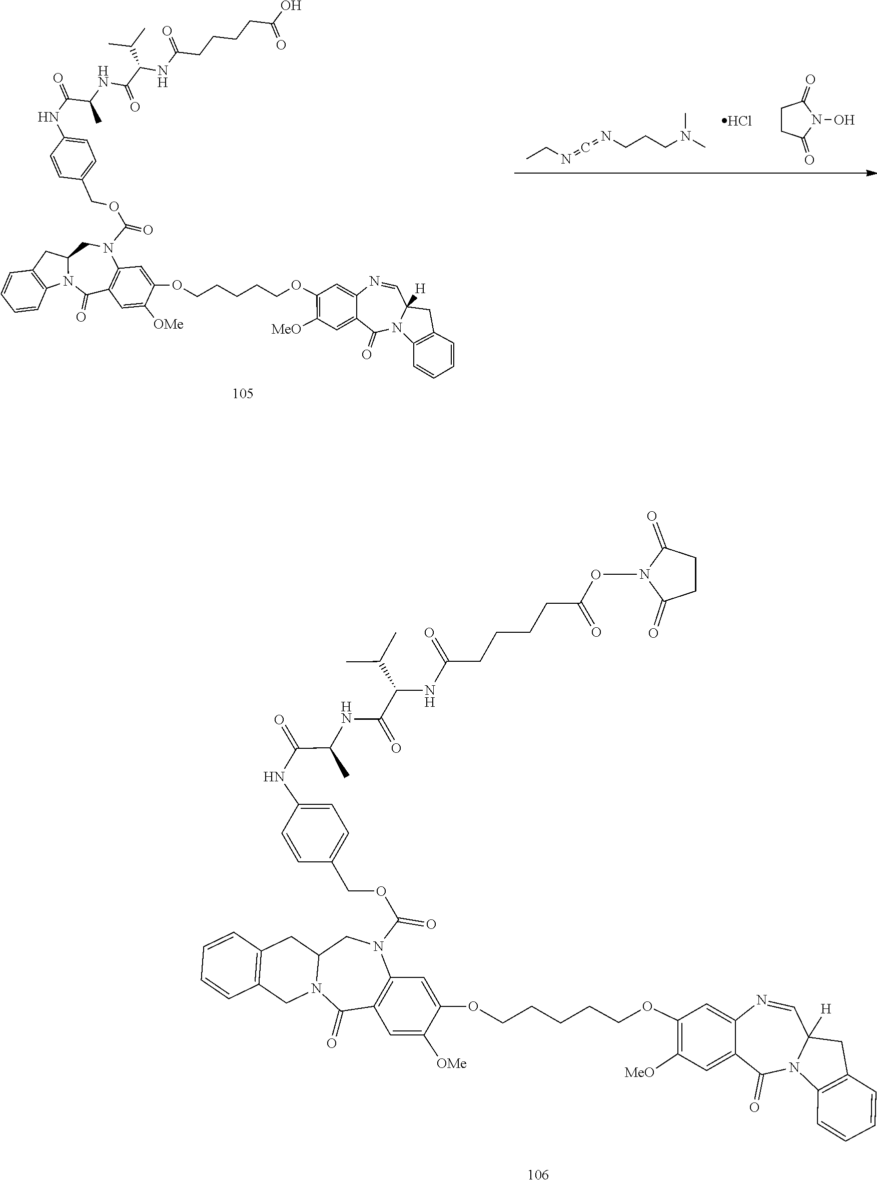 [SMILES: COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NC(=O)CCCCC(=O)O)C(C)C)cc1)N=C[C@@H]1Cc3ccccc3N1C2=O ; CCN=C=NCCCN(C)C ; Cl ; O=C1CCC(=O)N1O ; COc1cc2c(cc1OCCCCCOc1cc3c(cc1OC)C(=O)N1Cc4ccccc4CC1CN3C(=O)OCc1ccc(NC(=O)[C@H](C)NC(=O)[C@@H](NC(=O)CCCCC(=O)ON3C(=O)CCC3=O)C(C)C)cc1)N=CC1Cc3ccccc3N1C2=O]

To a solution of 6-(((S)-1-(((S)-1-((4-((((S)-2-methoxy-3-((5-(((S)-8-methoxy-6-oxo-12a,13-dihydro-6H-benzo[5,6][1,4]diazepino[1,2-a]indol-9-yl)oxy)pentyl)oxy)-14-oxo-5,6,6a,7,12,14-hexahydrobenzo[5,6][1,4]diazepino[1,2-b]isoquinoline-5-carbonyl)oxy)methyl)phenyl)amino)-1-oxopropan-2-yl)amino)-3-methyl-1-oxobutan-2-yl)amino)-6-oxohexanoic acid (70 mg, 0.062 mmol) and N-hydroxy succinimide (21.57 mg, 0.187 mmol) in anhydrous dichloromethane (1.25 ml) was added 1-ethyl-3-(3-dimethylaminopropyl) carbodiimide hydrochloride (59.9 mg, 0.312 mmol). The reaction stirred at ambient temperature under nitrogen for 90 minutes upon which it was diluted with dichloromethane and washed with water. The organic layer was dried with anhydrous magnesium sulfate, filtered and concentrated. The crude white solid was purified by RP-HPLC (C18 Kromasil, acetonitrile/deionized water, 50-65% over 30 min). Fractions containing product were frozen and lyophilized to yield pure compound 106 as a bright white solid (48 mg, y=63% yield). MS (m/z): 1121.2 (M+1)⁺. UPLC=1.94 min (2.5 min method).

### Example 15. Synthesis of Compound 108

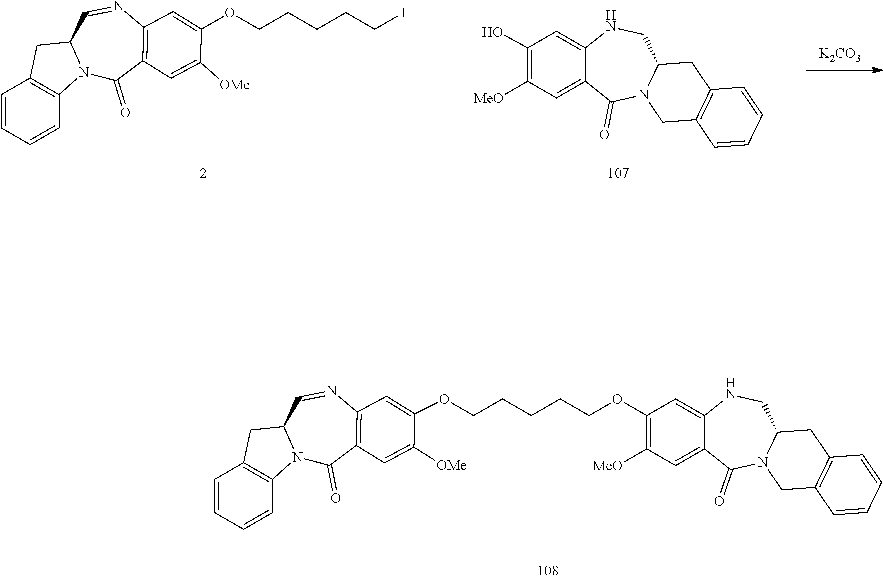 [SMILES: COc1cc2c(cc1OCCCCCI)N=C[C@@H]1Cc3ccccc3N1C2=O ; COc1cc2c(cc1O)NC[C@@H]1Cc3ccccc3CN1C2=O ; "K2CO3" ; COc1cc2c(cc1OCCCCCOc1cc3c(cc1OC)C(=O)N1Cc4ccccc4C[C@H]1CN3)N=C[C@@H]1Cc3ccccc3N1C2=O]

To a solution of compound 2 (48.7 mg, 0.099 mmol) and (S)-3-hydroxy-2-methoxy-6,6a,7,12-tetrahydrobenzo[5,6][1,4]diazepino[1,2-b]isoquinolin-14(5H)-one (28 mg, 0.090 mmol) in anhydrous N,N-dimethylacetamide (601 μl) was added anhydrous potassium carbonate (18.70 mg, 0.135 mmol). The mixture stirred for 18 hours at room temperature. Upon completion of the reaction water was added and the resulting solid was filtered, redissolved in dichloromethane, and washed with water. The organic layer was dried with anhydrous magnesium sulfate, filtered and concentrated. The crude material was purified by silica gel chromatography in methanol/dichloromethane to obtain compound 108 (31 mg, y=50%). MS (m/z): 673.4 (M+1)⁺. UPLC=1.63 min (2.5 min method).

### Example 16. Synthesis of Compound 110

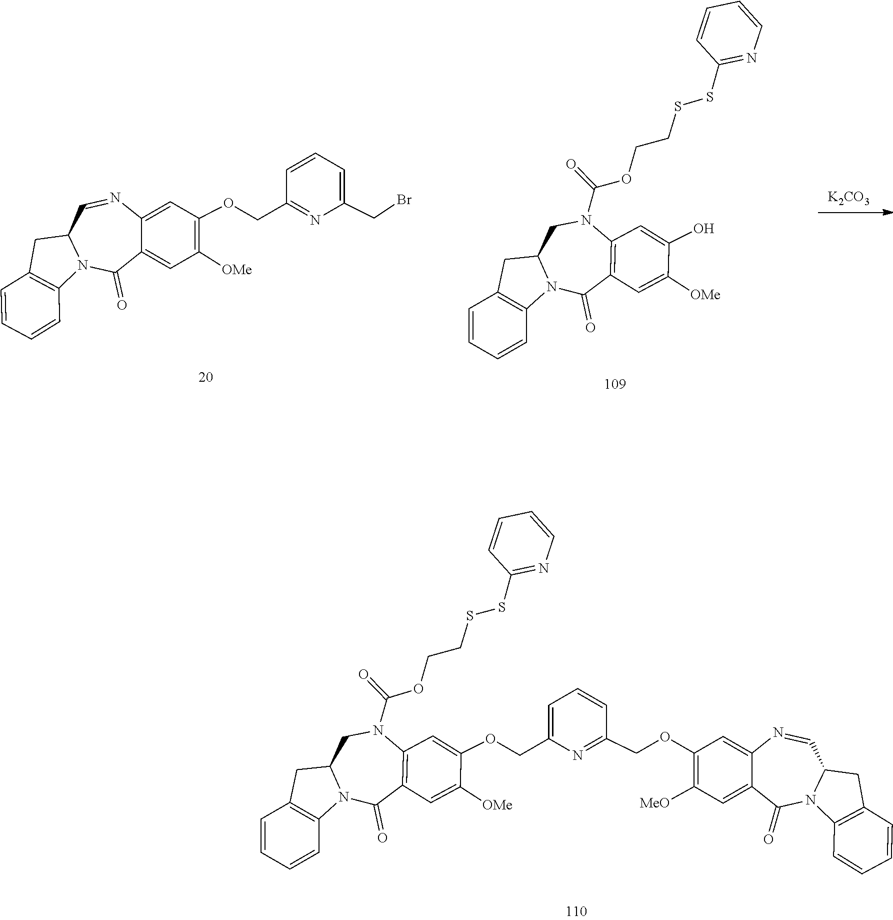 [SMILES: COc1cc2c(cc1OCc1cccc(CBr)n1)N=C[C@@H]1Cc3ccccc3N1C2=O ; COc1cc2c(cc1O)N(C(=O)OCCSSc1ccccn1)C[C@@H]1Cc3ccccc3N1C2=O ; "K2CO3" ; COc1cc2c(cc1OCc1cccc(COc3cc4c(cc3OC)C(=O)N3c5ccccc5C[C@H]3CN4C(=O)OCCSSc3ccccn3)n1)N=C[C@@H]1Cc3ccccc3N1C2=O]

To a solution of compound 20 (101 mg, 0.212 mmol) and 2-(pyridin-2-yldisulfanyl)ethyl (S)-9-hydroxy-8-methoxy-6-oxo-12a,13-dihydro-6H-benzo[5,6][1,4]diazepino[1,2-a]indole-11(12H)-carboxylate (90 mg, 0.177 mmol) in anhydrous N,N-Dimethylacetamide (1.76 ml) was added potassium carbonate (48.8 mg, 0.353 mmol) and the reaction was stirred at ambient temperature for 18 hours under nitrogen. The reaction mixture was diluted with water and the resulting white solid was filtered. Then the solid was dissolved in dichloromethane, transferred to a separatory funnel and washed with water. The organic layer was dried over anhydrous magnesium sulfate and concentrated in vacuo. One third of the crude material was purified via RP-HPLC (C18 Kromasil, acetonitrile/deionized water, 50-65% over 30 min). Fractions containing product were frozen and lyophilized to obtain compound 110 (18 mg, y=33%) as a white solid. MS (m/z): 907.9 (M+1)⁺. UPLC=1.91 min (2.5 min method).

### Example 17. Synthesis of Compound 117

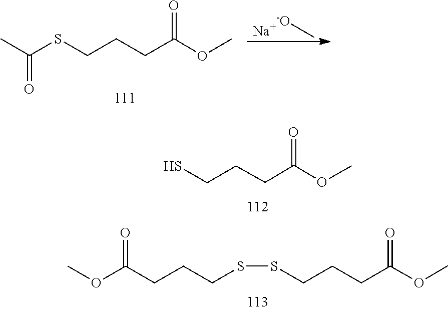

To a solution of methyl 4-(acetylthio)butanoate (2.6 g, 14.75 mmol) in methanol (369 ml) was added sodium methoxide (0.895 g, 16.23 mmol) and the resulting clear orange solution was stirred at room temperature for 3.5 hours. The mixture was concentrated to dryness then redissolved in dichloromethane. The organic layer was washed three times with water and then dried with anhydrous sodium sulfate, filtered and concentrated to obtain 2:1 thiol/disulfide mixture as a yellow oil (0.82 g, 40%) that was used without further purification. H NMR (400 Hz, CDCl₃): δ 33.67 (s, 6H), 2.71 (t, J=7.2 Hz, 2H), 2.61-54 (m, 2H), 2.48-2.42 (m, 4H), 2.05-1.99 (m, 2H), 1.98-1.91 (m, 2H), 1.33 (t, J=8.0 Hz, 1H).

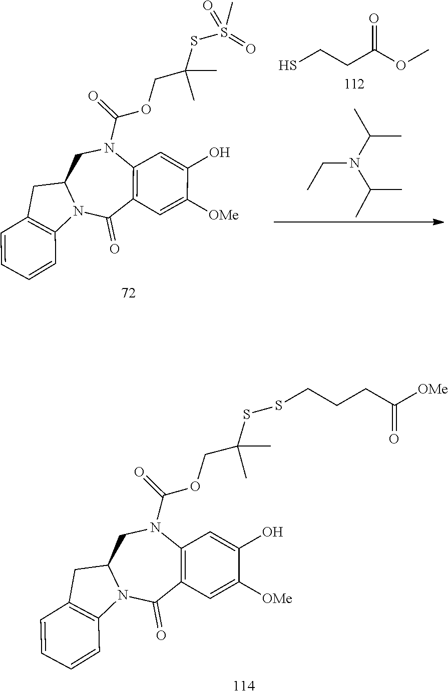

To a solution of 2-methyl-2-((methylsulfonyl)thio)propyl (S)-9-hydroxy-8-methoxy-6-oxo-12a,13-dihydro-6H-benzo[5,6][1,4]diazepino[1,2-a]indole-11(12H)-carboxylate (670 mg, 1.323 mmol) in anhydrous dichloromethane (13 mL) was added Diisopropylethylamine (0.461 mL, 2.65 mmol) and methyl 3-mercaptopropanoate (477 mg, 1.984 mmol). The mixture stirred at room temperature under nitrogen over 18 hours and was then diluted with dichloromethane and washed with water. The organic layer was dried over anhydrous magnesium sulfate, filtered and concentrated. The crude material was purified by silica gel chromatography in ethyl acetate/Hexanes) to yield compound 114 (0.54 g, y=73%) as a white crystalline solid. MS (m/z): 561.3 (M+1)⁺559.3 (M−1)⁻ UPLC=1.67 min (2.5 min method). ¹H NMR (400 Hz, d₆-DMSO): δ 9.85 (s, 1H), 8.02 (d, J=8.0 Hz, 1H), 7.28 (d, J=7.2 Hz, 1H), 7.22 (t, J=7.6 Hz, 1H), 7.12 (s, 1H), 7.06 (t, J=7.2 Hz, 1H), 6.77 (bs, 1H), 4.38-4.29 (m, 1H), 4.02 (t, J=12.4 Hz, 2H), 3.82 (s, 3H), 3.57 (s, 3H), 3.39-3.33 (m, 1H), 2.89 (d, J=16.0Hz, 1H), 2.59 (t, J=6.8 Hz, 2H), 2.35 (t, J=7.2 Hz, 2H), 1.81-1.76 (m, 2H), 1.17-1.05 (m, 6H).

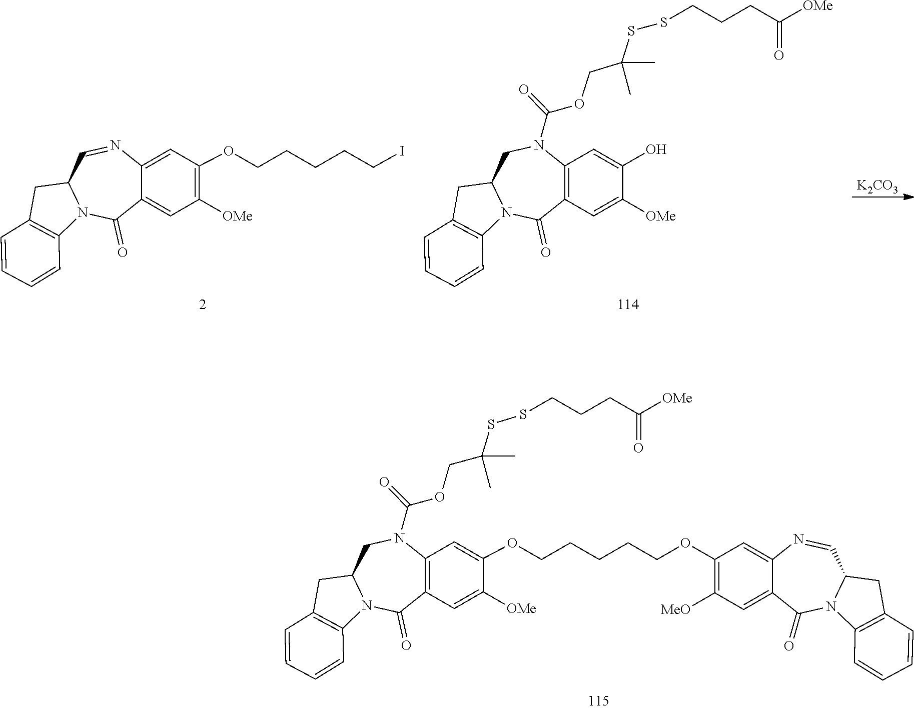 [SMILES: COc1cc2c(cc1OCCCCCI)N=C[C@@H]1Cc3ccccc3N1C2=O ; COC(=O)CCCSSC(C)(C)COC(=O)N1C[C@@H]2Cc3ccccc3N2C(=O)c2cc(OC)c(O)cc21 ; "K2CO3" ; COC(=O)CCCSSC(C)(C)COC(=O)N1C[C@@H]2Cc3ccccc3N2C(=O)c2cc(OC)c(OCCCCCOc3cc4c(cc3OC)C(=O)N3c5ccccc5C[C@H]3C=N4)cc21]

To a suspension of compound 2 (105 mg, 0.193 mmol) and 2-((4-methoxy-4-oxobutyl)disulfanyl)-2-methylpropyl (S)-9-hydroxy-8-methoxy-6-oxo-12a,13-dihydro-6H-benzo[5,6][1,4]diazepino[1,2-a]indole-11(12H)-carboxylate (90 mg, 0.161 mmol) in anhydrous N,N-Dimethylacetamide (1.60 ml) was added potassium carbonate (44.4 mg, 0.321 mmol) at room temperature under nitrogen. The mixture stirred for 18 hours after which it was quenched with water. The resulting white solid was filtered and then re-dissolved in dichloromethane, transferred to a separatory funnel and washed with water. The organic layer was dried over anhydrous magnesium sulfate, filtered, and concentrated in vacuo. The crude material was purified by silica gel chromatography in methanol/dichloromethane to obtain compound 115 (0.15 g, y=100%). MS (m/z): 941.7 (M+1)⁺ UPLC=1.95 min (2.5 min method).

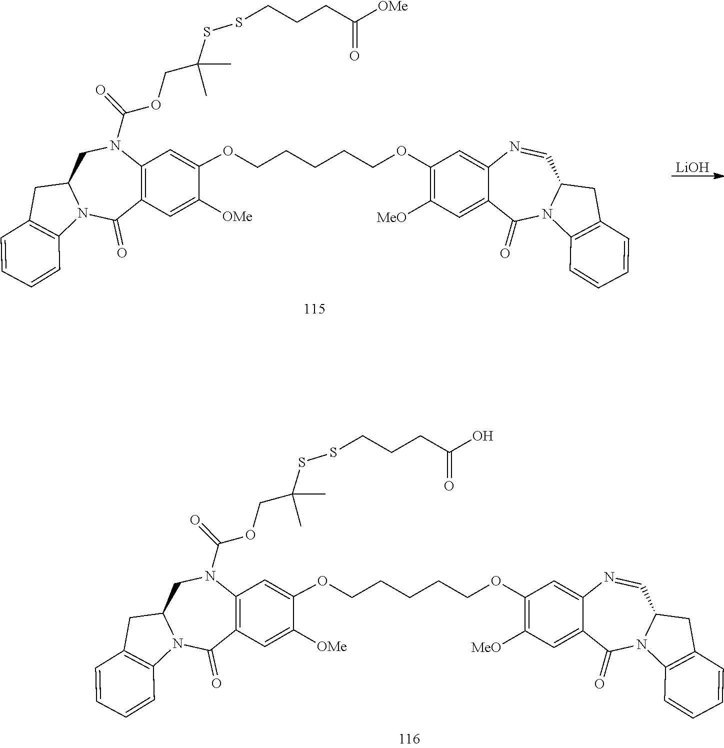 [SMILES: COC(=O)CCCSSC(C)(C)COC(=O)N1C[C@@H]2Cc3ccccc3N2C(=O)c2cc(OC)c(OCCCCCOc3cc4c(cc3OC)C(=O)N3c5ccccc5C[C@H]3C=N4)cc21 ; "LiOH" ; COc1cc2c(cc1OCCCCCOc1cc3c(cc1OC)C(=O)N1c4ccccc4C[C@H]1CN3C(=O)OCC(C)(C)SSCCCC(=O)O)N=C[C@@H]1Cc3ccccc3N1C2=O]

To a cooled solution of 2-((4-methoxy-4-oxobutyl)disulfaneyl)-2-methylpropyl (S)-8-methoxy-9-((5-(((S)-8-methoxy-6-oxo-12a,13-dihydro-6H-benzo[5,6][1,4]diazepino[1,2-a]indol-9-yl)oxy)pentyl)oxy)-6-oxo-12a,13-dihydro-6H-benzo[5,6][1,4]diazepino[1,2-a]indole-11(12H)-carboxylate—methane (180 mg, 0.167 mmol) in anhydrous Tetrahydrofuran (6.25 ml) and deionized water (2.08 ml) was added lithium hydroxide (19.97 mg, 0.834 mmol) at 0° C. The reaction stirred from 0° C. to room temperature over three hours upon which it was diluted with dichloromethane and deionized water. The mixture was acidified to pH=3 with Hydrochloric acid (0.5 M aqueous, 1 mL) and extracted with dichloromethane (2×20 ml). The organic layer was washed with water, dried over anhydrous magnesium sulfate, filtered and concentrated. The crude was carried on without further purification, assuming 100% yield. MS (m/z): 927.6 (M+1)*925.6 (M−1)⁻. UPLC=1.76 min (2.5 min method).

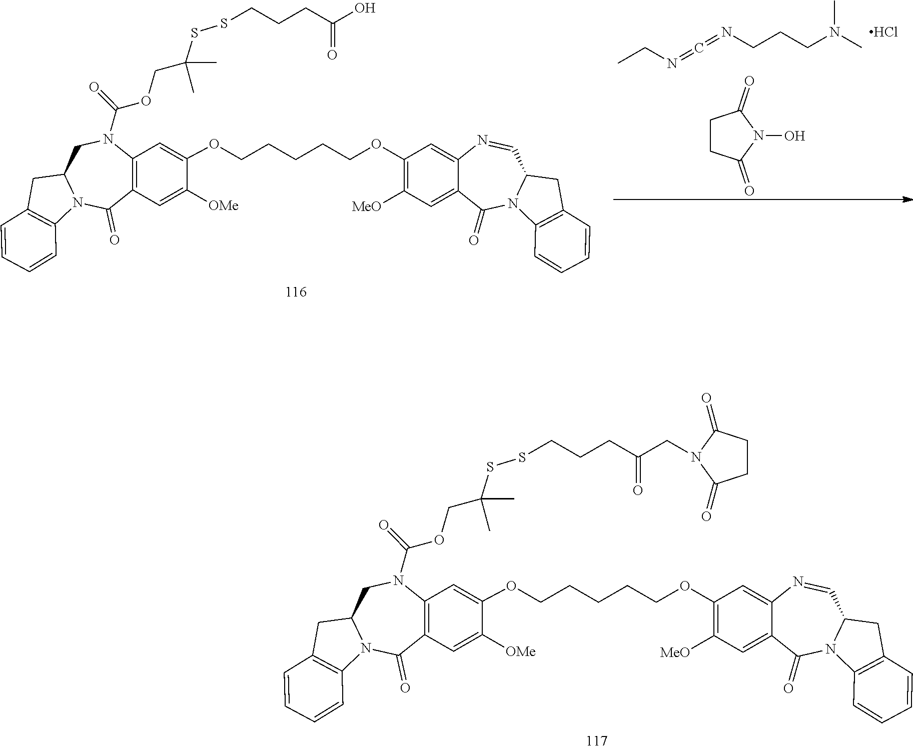

To a solution of 4-((1-(((S)-8-methoxy-9-((5-(((S)-8-methoxy-6-oxo-12a,13-dihydro-6H-benzo[5,6][1,4]diazepino[1,2-a]indol-9-yl)oxy)pentyl)oxy)-6-oxo-11,12,12a,13-tetrahydro-6H-benzo[5,6][1,4]diazepino[1,2-a]indole-11-carbonyl)oxy)-2-methylpropan-2-yl)disulfanyl)butanoic acid (150 mg, 0.165 mmol) and N-hydroxy succinimide (57.0 mg, 0.495 mmol) in anhydrous dichloromethane (3.30 ml) was added 1-ethyl-3-(3-dimethylaminopropyl) carbodiimide hydrochloride (158 mg, 0.825 mmol) and the mixture stirred for three hours at room temperature under nitrogen. The reaction was diluted with dichloromethane and washed with water. The organic layer was dried with anhydrous magnesium sulfate, filtered and concentrated. Half of the crude material was purified by RP-HPLC (C18, acetonitrile/deionized water). Fractions containing DP were frozen and lyophilized to obtain compound 117 (38 mg, y=46%) as a white solid. MS (m/z): 1006.7 (M+1)⁺. UPLC=1.89 min (2.5 min method).

### Example 18. Synthesis of Compound 118

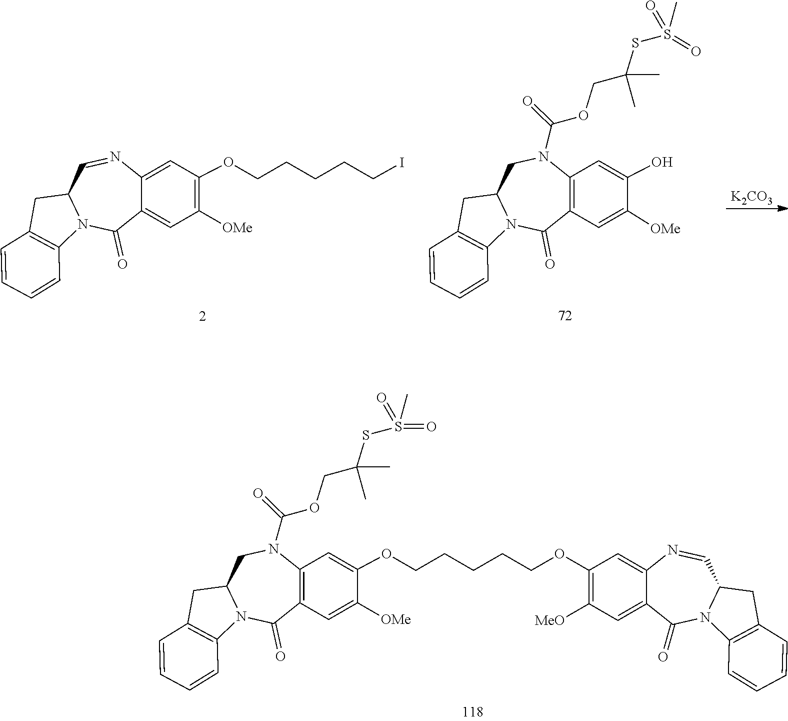

To a suspension of compound 2 (116 mg, 0.213 mmol) and 2-methyl-2-((methylsulfonyl)thio)propyl (S)-9-hydroxy-8-methoxy-6-oxo-12a,13-dihydro-6H-benzo[5,6][1,4]diazepino[1,2-a]indole-11(12H)-carboxylate (90 mg, 0.178 mmol) in anhydrous N,N-Dimethylacetamide (1.77 ml) was added anhydrous potassium carbonate (49.1 mg, 0.355 mmol) and the reaction was stirred for 18 hours at room temperature under nitrogen. water was added to the reaction mixture and the resulting white solid was filtered, re-dissolved in dichloromethane, transferred to a separatory funnel, and washed with water. The organic layer was dried over anhydrous magnesium sulfate and concentrated in vacuo. Half of the crude material was purified via RP-HPLC (C18 Kromasil, acetonitrile/deionized water, 55-65% over 30 min). Pure fractions were extracted with dichloromethane, dried over anhydrous magnesium sulfate, filtered and concentrated to obtain compound 118 (45 mg, y=58%) as a white solid.

MS (m/z): 869.6 (M+1)⁺. UPLC=1.85 min (2.5 min method).

### Example 19. Synthesis of Compound 121

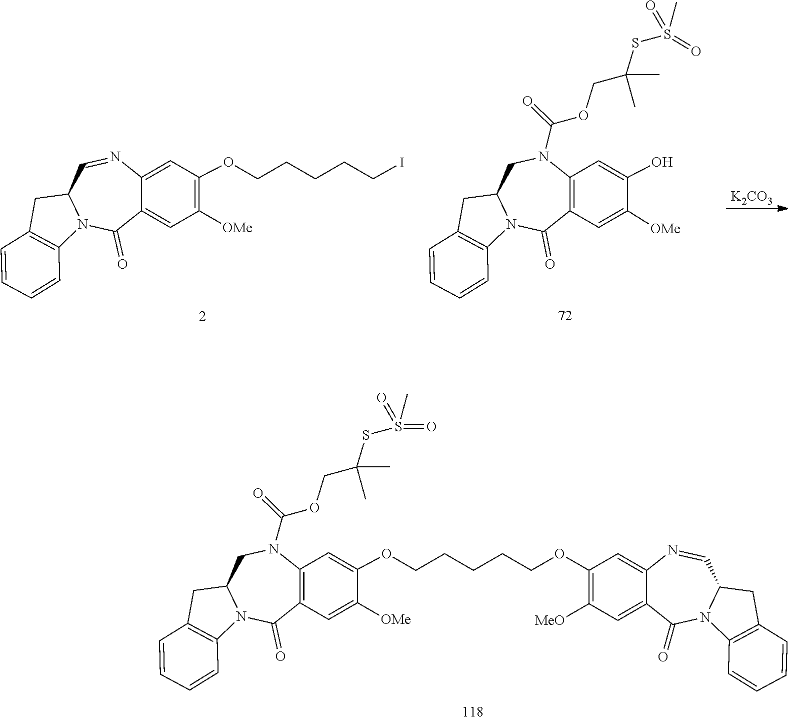

To a suspension of Compound 20 (132 mg, 0.235 mmol) and 2-((4-methoxy-4-oxobutyl)disulfanyl)-2-methylpropyl (S)-9-hydroxy-8-methoxy-6-oxo-12a,13-dihydro-6H-benzo[5,6][1,4]diazepino[1,2-a]indole-11(12H)-carboxylate (110 mg, 0.196 mmol) in anhydrous N,N-Dimethylacetamide (1.96 ml) was added anhydrous potassium carbonate (54.2 mg, 0.392 mmol) and the reaction was stirred for 18 hours at room temperature under nitrogen. water was added to the reaction mixture and the resulting white solid was filtered, re-dissolved in dichloromethane and washed with water. The organic layer was dried over anhydrous magnesium sulfate and concentrated in vacuo. The crude material was purified by flash chromatography (methanol/dichloromethane) to give compound 119 (172 mg, y=90%). MS (m/z): 976.7 (M+1)⁺. UPLC=1.91 min (2.5 min method).

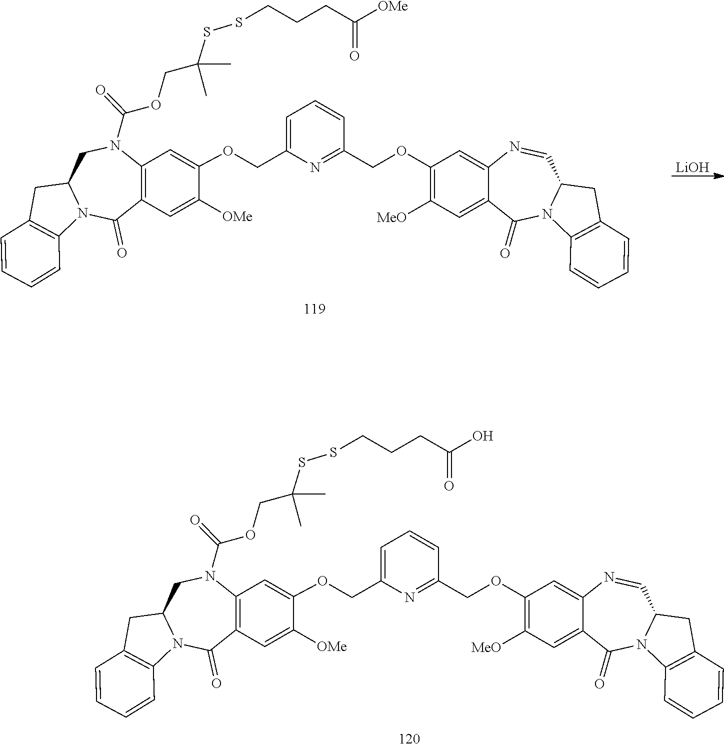 [SMILES: COC(=O)CCCSSC(C)(C)COC(=O)N1C[C@@H]2Cc3ccccc3N2C(=O)c2cc(OC)c(OCc3cccc(COc4cc5c(cc4OC)C(=O)N4c6ccccc6C[C@H]4C=N5)n3)cc21 ; "LiOH" ; COc1cc2c(cc1OCc1cccc(COc3cc4c(cc3OC)C(=O)N3c5ccccc5C[C@H]3CN4C(=O)OCC(C)(C)SSCCCC(=O)O)n1)N=C[C@@H]1Cc3ccccc3N1C2=O]

Compound 119 (200 mg, 0.177 mmol) in anhydrous Tetrahydrofuran (6.62 ml) and water (2.20 ml) was cooled to 0° C. in an ice bath and lithium hydroxide (21.14 mg, 0.883 mmol) was added. The reaction stirred from 0° C. to room temperature for three hours after which it was diluted with dichloromethane and deionized water. The mixture was acidified to pH=3 with hydrochloric acid (0.5 M aqueous, 1 ml) and then extracted with dichloromethane (2×20 ml). The organic layer was washed with water, dried with anhydrous magnesium sulfate, filtered and concentrated to obtain crude compound 120 that was used without purification in the next step. MS (m/z): 962.6 (M+1)⁺960.7 (M−1)⁻. UPLC=1.73 min (2.5 min method).

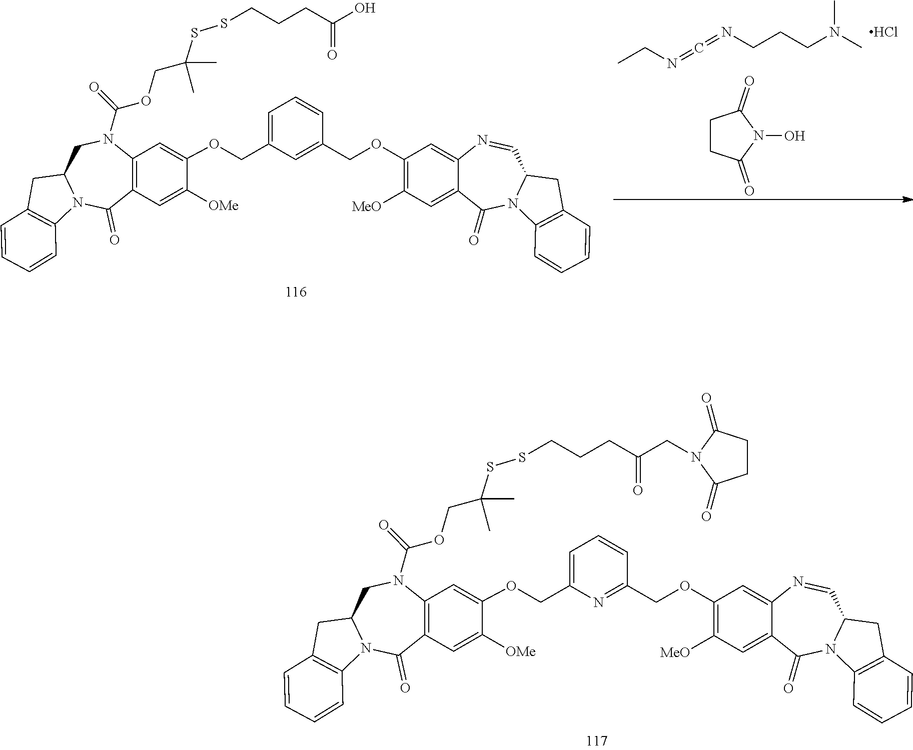

To a solution of 4-((1-(((S)-8-methoxy-9-((6-((((S)-8-methoxy-6-oxo-12a,13-dihydro-6H-benzo[5,6][1,4]diazepino[1,2-a]indol-9-yl)oxy)methyl)pyridin-2-yl)methoxy)-6-oxo-11,12,12a,13-tetrahydro-6H-benzo[5,6][1,4]diazepino[1,2-a]indole-11-carbonyl)oxy)-2-methylpropan-2-yl)disulfanyl)butanoic acid (155 mg, 0.164 mmol) and N-hydroxy succinimide (56.7 mg, 0.493 mmol) in anhydrous dichloromethane (3284 μl) was added 1-ethyl-3-(3-dimethylaminopropyl) carbodiimide hydrochloride (157 mg, 0.821 mmol). The reaction stirred at room temperature for three hours and was then diluted with dichloromethane and washed with water. The organic layer was dried with anhydrous magnesium sulfate, filtered and concentrated. Half of the resulting crude material was purified by RP-HPLC (Kromasil C18, acetonitrile/deionized water). Fractions containing product were frozen and lyophilized to obtain compound 121 (43.5 mg, y=50%) as a white solid. MS (m/z): 1041.7 (M+1)⁺. UPLC=1.85 min (2.5 min method).

### Example 20. Synthesis of Compound 122

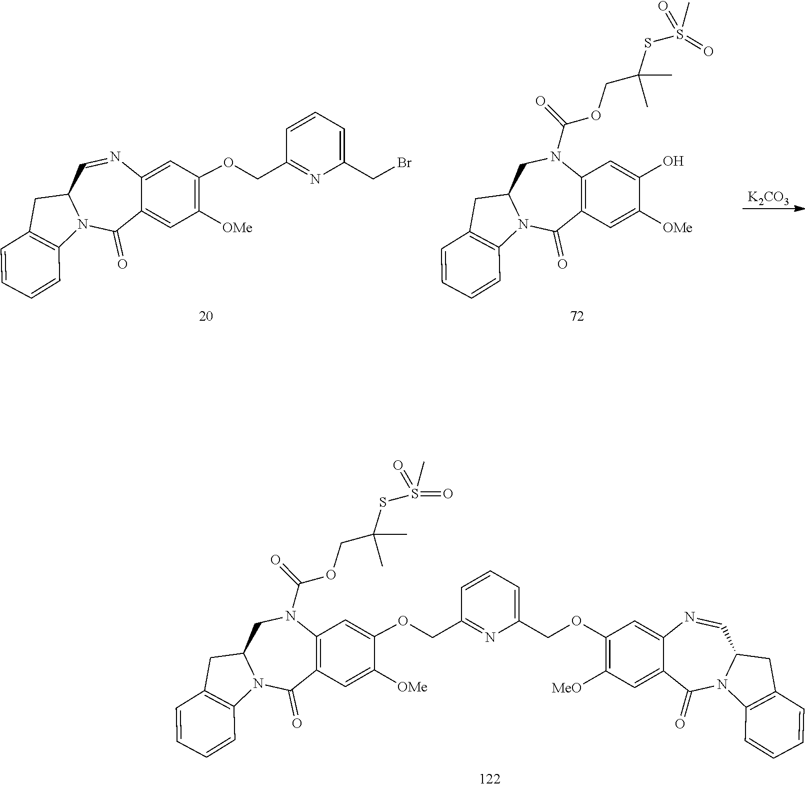

To a suspension of Compound 20 (71.4 mg, 0.149 mmol) and 2-methyl-2-((methylsulfonyl)thio)propyl (S)-9-hydroxy-8-methoxy-6-oxo-12a,13-dihydro-6H-benzo[5,6][1,4]diazepino[1,2-a]indole-11(12H)-carboxylate (63 mg, 0.124 mmol) in anhydrous N,N-Dimethylacetamide (1.24 ml) was added anhydrous potassium carbonate (34.4 mg, 0.249 mmol) and the reaction was stirred for 18 hours at room temperature under nitrogen. water was added to the reaction mixture and the resulting white solid was filtered, redissolved in dichloromethane, transferred to a separatory funnel, and washed with water. The organic layer was dried over anhydrous magnesium sulfate, filtered and concentrated in vacuo. The crude was purified via RP-HPLC (C18 Kromasil, acetonitrile/Deionized water, 50-70% over 30 min). Fractions containing DP were frozen and lyophilized to obtain compound 122 as a white solid (64 mg, y=57%). MS (m/z): 904.6 (M+1)⁺. UPLC=1.85 min (2.5 min method).

### Example 21. Synthesis of Compound 123

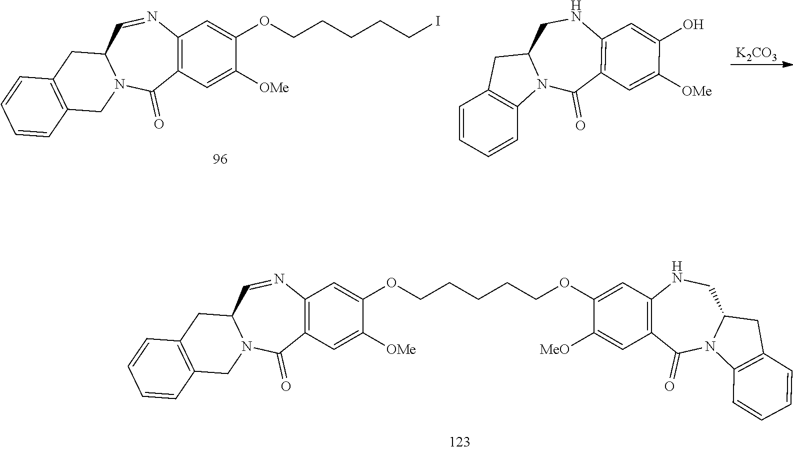

To a solution of compound 96 and (S)-9-hydroxy-8-methoxy-11,12,12a,13-tetrahydro-6H-benzo[5,6][1,4]diazepino[1,2-a]indol-6-one (78 mg, 0.262 mmol) in anhydrous N,N-dimethylacetamide (1.58 ml) was added potassium carbonate (49.3 mg, 0.357 mmol). The reaction was stirred for 18 hours at room temperature after which it was diluted with water. The resulting solid was filtered and then redissolved in dichloromethane and washed with water. The organic layer was dried with anhydrous magnesium sulfate, filtered and concentrated. The crude material was purified by RP-HPLC (acetonitrile/deionized water, 55-75% over 30 min) Fractions containing product were combined and extracted with dichloromethane. The organic layer was dried with anhydrous magnesium sulfate, filtered and concentrated to give compound 123 (75 mg, y=46%). MS (m/z): 673.6 (M+1)⁺. UPLC=1.66 min (2.5 min method).

### Example 22. Synthesis of Compound 126

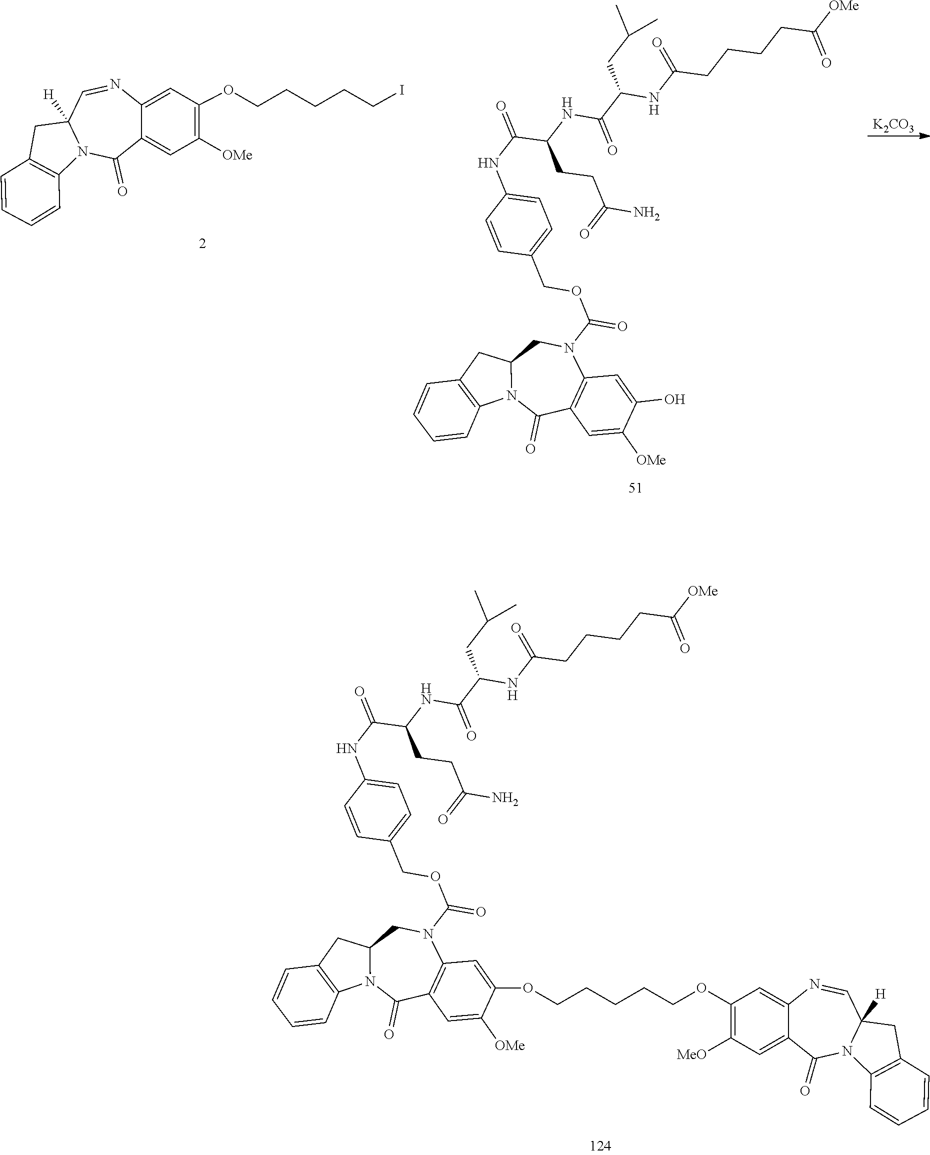 [SMILES: COc1cc2c(cc1OCCCCCI)N=C[C@@H]1Cc3ccccc3N1C2=O ; COC(=O)CCCCC(=O)N[C@@H](CC(C)C)C(=O)N[C@@H](CCC(N)=O)C(=O)Nc1ccc(COC(=O)N2C[C@@H]3Cc4ccccc4N3C(=O)c3cc(OC)c(O)cc32)cc1 ; "K2CO3" ; COC(=O)CCCCC(=O)N[C@@H](CC(C)C)C(=O)N[C@@H](CCC(N)=O)C(=O)Nc1ccc(COC(=O)N2C[C@@H]3Cc4ccccc4N3C(=O)c3cc(OC)c(OCCCCCOc4cc5c(cc4OC)C(=O)N4c6ccccc6C[C@H]4C=N5)cc32)cc1]

To a solution of 4-((S)-5-amino-2-((S)-2-(6-methoxy-6-oxohexanamido)-4-methylpentanamido)-5-oxopentanamido)benzyl (S)-9-hydroxy-8-methoxy-6-oxo-12a,13-dihydro-6H-benzo[5,6][1,4]diazepino[1,2-a]indole-11(12H)-carboxylate (80 mg, 0.097 mmol) and compound 2 (56.8 mg, 0.116 mmol) in anhydrous N,N-Dimethylacetamide (965 μl) was added potassium carbonate (26.7 mg, 0.193 mmol) and the reaction was stirred for 18 hours at room temperature under nitrogen. water was added to the reaction mixture and the resulting solid was filtered, then redissolved in 20% methanol/dichloromethane, transferred to a separatory funnel, and washed with water. The organic layer was dried over anhydrous magnesium sulfate and concentrated in vacuo. The crude material was purified via silica gel chromotography (12 g silica column, methanol/dichloromethane) to obtain compound 124 (70 mg, y=61%). MS (m/z): 1192.0 (M+1)⁺. UPLC=5.55 min (10 min method).

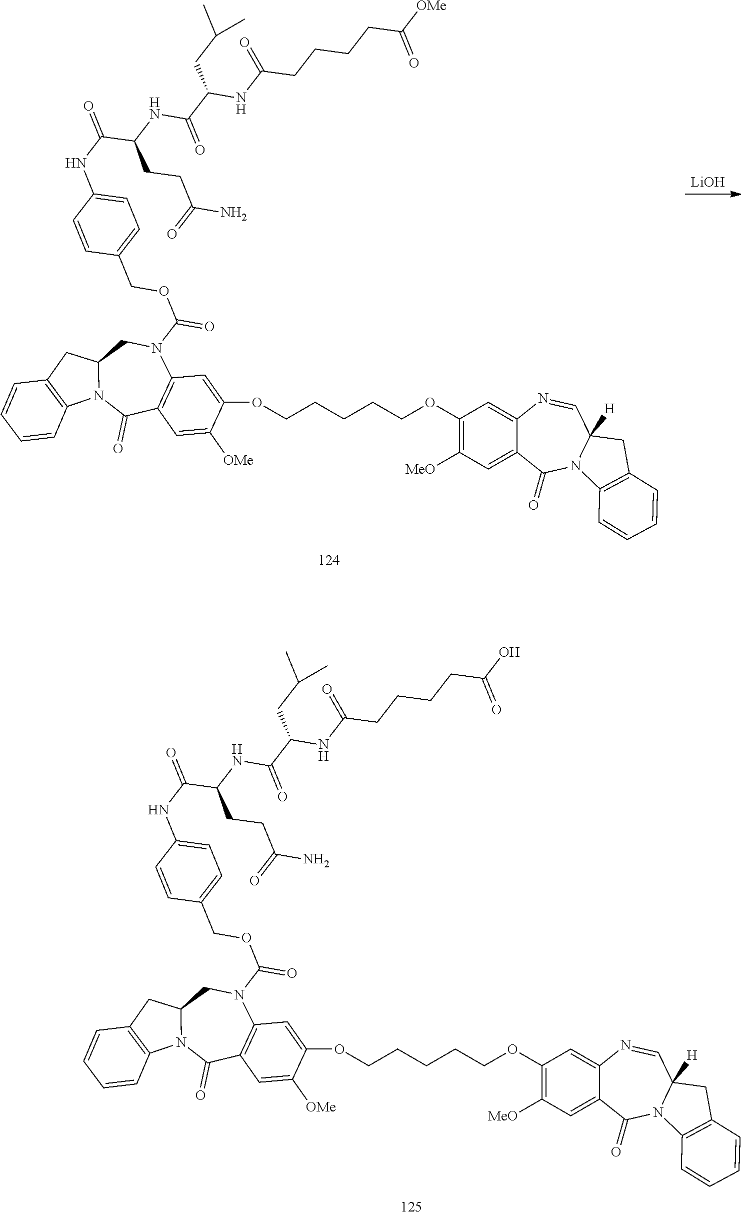 [SMILES: COC(=O)CCCCC(=O)N[C@@H](CC(C)C)C(=O)N[C@@H](CCC(N)=O)C(=O)Nc1ccc(COC(=O)N2C[C@@H]3Cc4ccccc4N3C(=O)c3cc(OC)c(OCCCCCOc4cc5c(cc4OC)C(=O)N4c6ccccc6C[C@H]4C=N5)cc32)cc1 ; "LiOH" ; COc1cc2c(cc1OCCCCCOc1cc3c(cc1OC)C(=O)N1c4ccccc4C[C@H]1CN3C(=O)OCc1ccc(NC(=O)[C@H](CCC(N)=O)NC(=O)[C@H](CC(C)C)NC(=O)CCCCC(=O)O)cc1)N=C[C@@H]1Cc3ccccc3N1C2=O]

To a solution of 4-((S)-5-amino-2-((S)-2-(6-methoxy-6-oxohexanamido)-4-methylpentanamido)-5-oxopentanamido)benzyl (S)-8-methoxy-9-((5-(((S)-8-methoxy-6-oxo-12a,13-dihydro-6H-benzo[5,6][1,4]diazepino [1,2-a]indol-9-yl)oxy)pentyl)oxy)-6-oxo-12a,13-dihydro-6H-benzo[5,6][1,4]diazepino[1,2-a]indole-11(12H)-carboxylate (60 mg, 0.050 mmol) in anhydrous Tetrahydrofuran (1.88 ml) and deionized water (630 μl) was added lithium hydroxide (3.62 mg, 0.151 mmol). After stirring for 90 minutes at room temperature the mixture was diluted with 30% methanol in dichloromethane and deionized water and acidified to pH=3 with hydrochloric acid (0.5 M aqueous). The mixture was extracted with 30% methanol/dichloromethane (3×50 mL). The organic layer was washed with water, dried with anhydrous magnesium sulfate, filtered through celite and evaporated. The crude product 125 was carried on without further purification, assuming 100% yield. MS (m/z): 1178.0 (M+1)⁺. UPLC=5.53 min (10 min method).

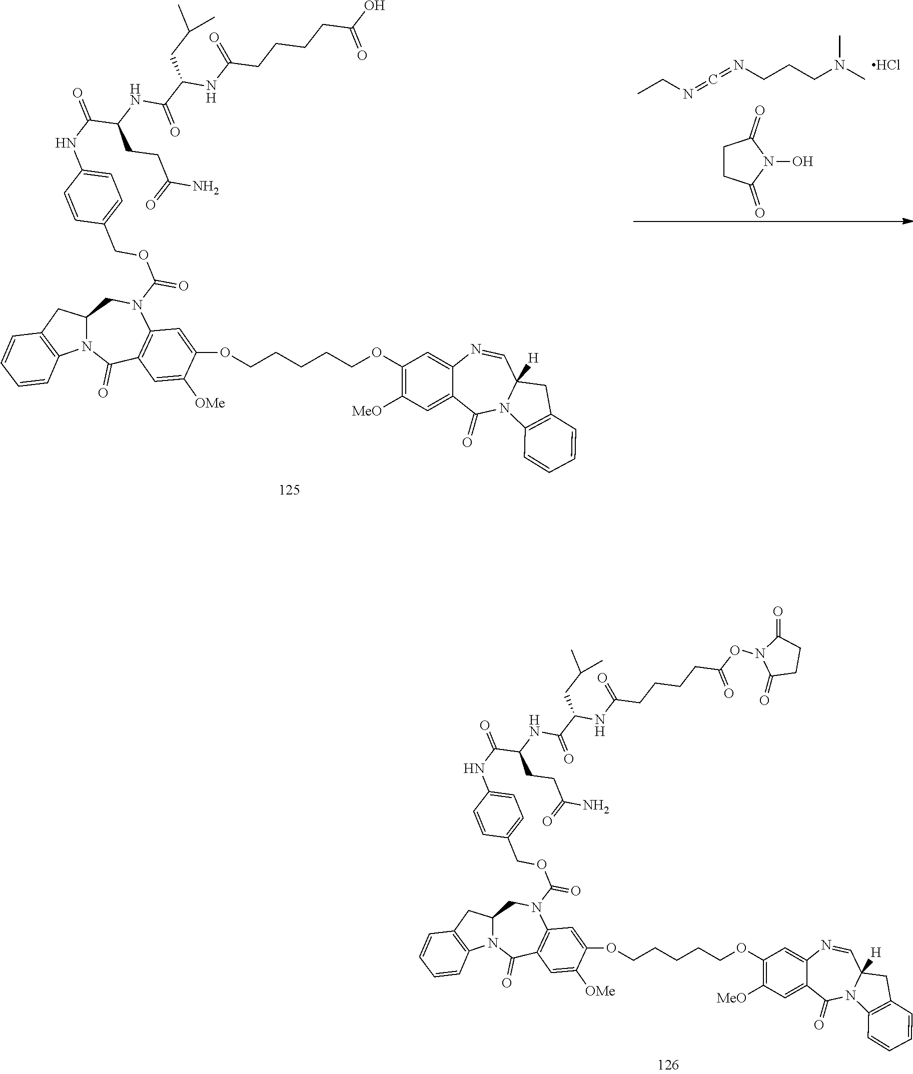

To a solution of compound 125 (58.9 mg, 0.05 mmol) and N-hydroxy succinimide (17.26 mg, 0.150 mmol) in anhydrous dichloromethane (750 μl) and anhydrous N,N-Dimethylformamide (250 μl) was added 1-ethyl-3-(3-dimethylaminopropyl) carbodiimide hydrochloride (47.9 mg, 0.250 mmol). The reaction was allowed to stir at room temperature under nitrogen for three hours, upon which the mixture was concentrated to remove dichloromethane. Acetonitrile and deionized water were added and the mixture was frozen and lyophilized. The crude material was purified by RP-HPLC (C18 Kromasil, acetonitrile/deionized water). Fractions containing DP were frozen and lyophilized as pure compound 126 (32 mg, y=51% over 2 steps). MS (m/z): 1275.0 (M+1)⁺. UPLC=5.77 min (10 min method).

### Example 23. Synthesis of Compound 134

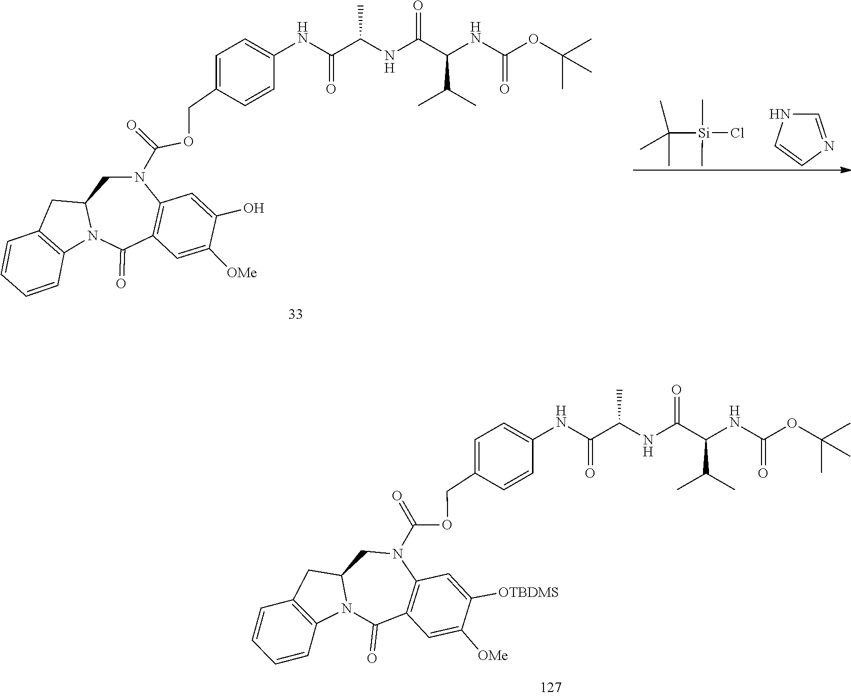

To a solution of 4-((S)-2-((S)-2-((tert-butoxycarbonyl)amino)-3-methylbutanamido)propanamido)benzyl (S)-9-hydroxy-8-methoxy-6-oxo-12a,13-dihydro-6H-benzo[5,6][1,4]diazepino[1,2-a]indole-11(12H)-carboxylate (0.28 g, 0.391 mmol) and t-butyldimethylsilyl chloride (0.077 g, 0.509 mmol) in anhydrous dimethylformamide (2.61 ml) was added Imidazole (0.067 g, 0.978 mmol). The reaction mixture stirred at ambient temperature for 20 hours upon which it was extracted with ethyl acetate. The organic layer was washed with water and brine solution, then dried with anhydrous magnesium sulfate, filtered and concentrated. The crude material was purified by silica gel chromatography in ethyl acetate/hexanes to give compound 127 as a white crystalline solid (0.30 g, y=92%). MS (m/z): 830.8 (M+1)⁺. UPLC=2.04 min (2.5 min method).

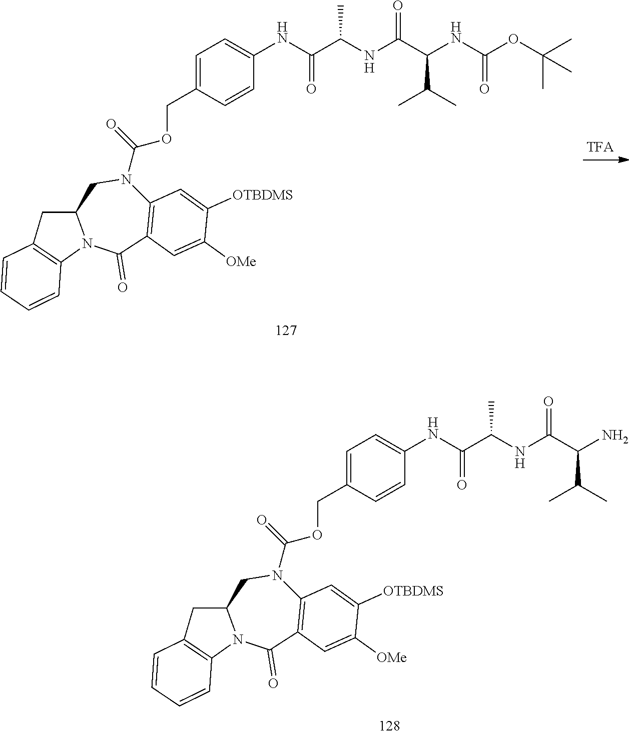 [SMILES: COc1cc2c(cc1O[Si](C)(C)C(C)(C)C)N(C(=O)OCc1ccc(NC(=O)[C@H](C)NC(=O)[C@@H](NC(=O)OC(C)(C)C)C(C)C)cc1)C[C@@H]1Cc3ccccc3N1C2=O ; COc1cc2c(cc1O[Si](C)(C)C(C)(C)C)N(C(=O)OCc1ccc(NC(=O)[C@H](C)NC(=O)[C@@H](N)C(C)C)cc1)C[C@@H]1Cc3ccccc3N1C2=O]

A solution of 4-((S)-2-((S)-2-((tert-butoxycarbonyl)amino)-3-methylbutanamido)propanamido)benzyl (S)-9-((tert-butyldimethylsilyl)oxy)-8-methoxy-6-oxo-12a,13-dihydro-6H-benzo[5,6][1,4]diazepino[1,2-a]indole-11(12H)-carboxylate (0.30 g, 0.361 mmol) in anhydrous dichloromethane (2.5 ml) was cooled to 0° C. in an ice bath. A fresh mixture of trifluoroacetic acid (1.807 ml) in anhydrous dichloromethane (2.5 ml) was added. After stirring for 30 minutes at 0° C. under nitrogen the reaction was poured into an ice/sodium bicarbonate mixture and then extracted with dichloromethane. The extracts were washed with brine, dried over anhydrous magnesium sulfate, filtered and concentrated. The crude compound 128 was carried on without further purification, assuming 100% yield. MS (m/z): 730.6 (M+1)⁺. UPLC=1.53 min (2.5 min method).

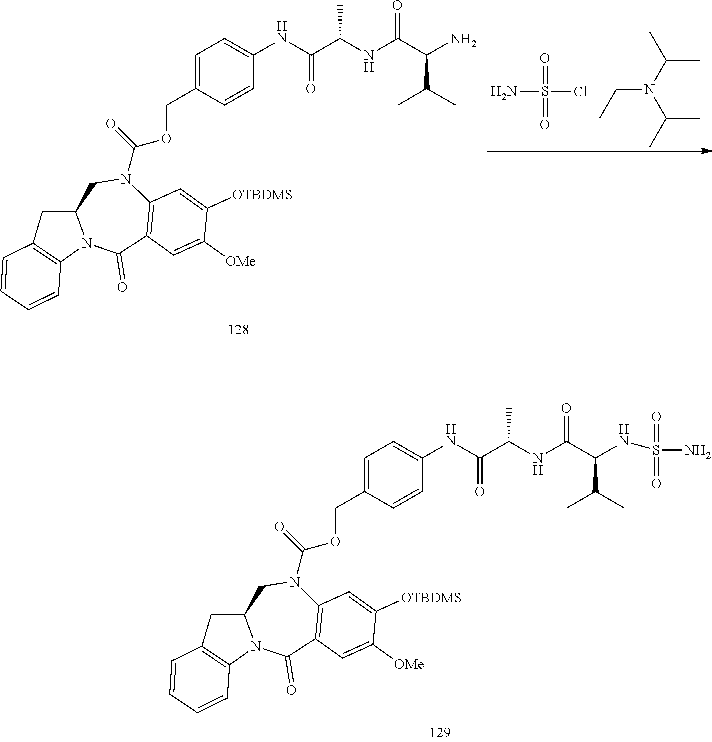

To a solution of 4-((S)-2-((S)-2-amino-3-methylbutanamido)propanamido)benzyl (S)-9-((tert-butyldimethylsilyl)oxy)-8-methoxy-6-oxo-12a,13-dihydro-6H-benzo[5,6][1,4]diazepino[1,2-a]indole-11(12H)-carboxylate (0.23 g, 0.315 mmol) in anhydrous N,N-dimethylacetamide (1.050 ml) was added Diisopropylethylamine (0.137 ml, 0.788 mmol). The reaction was cooled to 0° C. in an ice bath, then sulfamoyl chloride (0.073 g, 0.630 mmol) in anhydrous N,N-dimethylacetamide (0.25 ml) was added. The mixture stirred at 0° C. for 20 min then at room temperature for 20 hours after which it was quenched with saturated aqueous sodium bicarbonate solution. The mixture was extracted with ethyl acetate and the extracts were washed with water and brine. The organic layer was dried with anhydrous magnesium sulfate, filtered and concentrated. The crude was carried on without further purification, assuming 100% yield. MS (m/z): 809.8 (M+1)⁺ 807.8 (M−1)⁻. UPLC=1.84 min (2.5 min method).

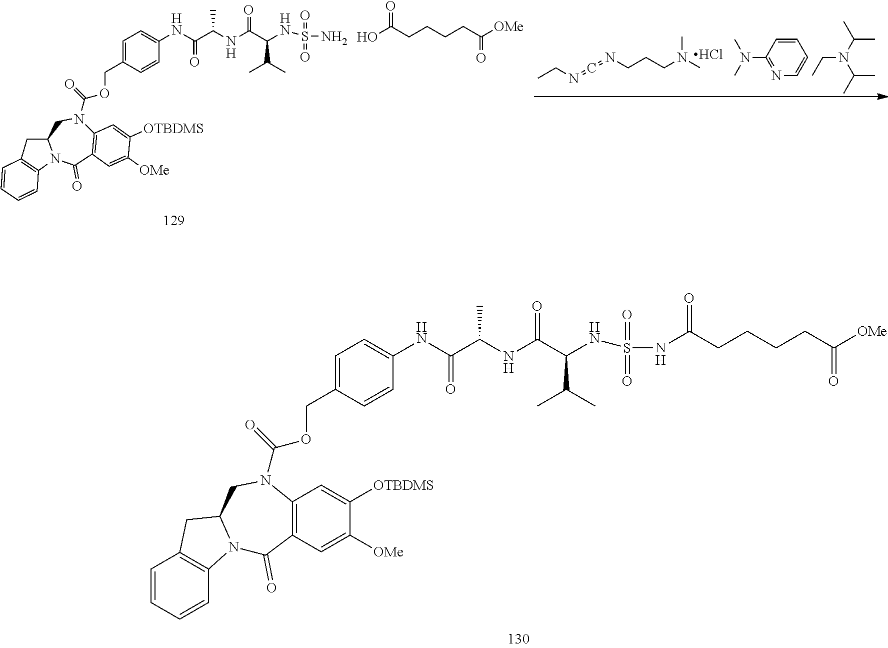

To a solution of 4-((S)-2-((S)-3-methyl-2-(sulfamoylamino)butanamido)propanamido)benzyl (S)-9-((tert-butyldimethylsilyl)oxy)-8-methoxy-6-oxo-12a,13-dihydro-6H-benzo[5,6][1,4]diazepino[1,2-a]indole-11(12H)-carboxylate (0.23 g, 0.284 mmol) and mono-methyl adipate (0.063 ml, 0.426 mmol) in anhydrous dichloromethane (1.895 ml) was added 1-ethyl-3-(3-dimethylaminopropyl) carbodiimide hydrochloride (0.082 g, 0.426 mmol), 4-dimethylaminopyridine (0.017 g, 0.142 mmol) and diisopropylethylamine (0.059 ml, 0.341 mmol) were added. The reaction mixture was stirred at ambient temperature for 20 hours, upon which it was diluted with dichloromethane and washed with water and brine. The organic layer was dried with anhydrous sodium sulfate and concentrated under reduced pressure. The crude material was purified by silica gel chromatography in methanol/dichloromethane) to yield 4-((S)-2-((S)-2-((N-(6-methoxy-6-oxohexanoyl)sulfamoyl)amino)-3-methylbutanamido)propanamido)benzyl (S)-9-((tert-butyldimethylsilyl)oxy)-8-methoxy-6-oxo-12a,13-dihydro-6H-benzo[5,6][1,4]diazepino[1,2-a]indole-11(12H)-carboxylate (86 mg, y=29% over 4 steps). MS (m/z): 952.0 (M+1)⁺949.9 (M−1)⁻. UPLC=1.92 min (2.5 min method).

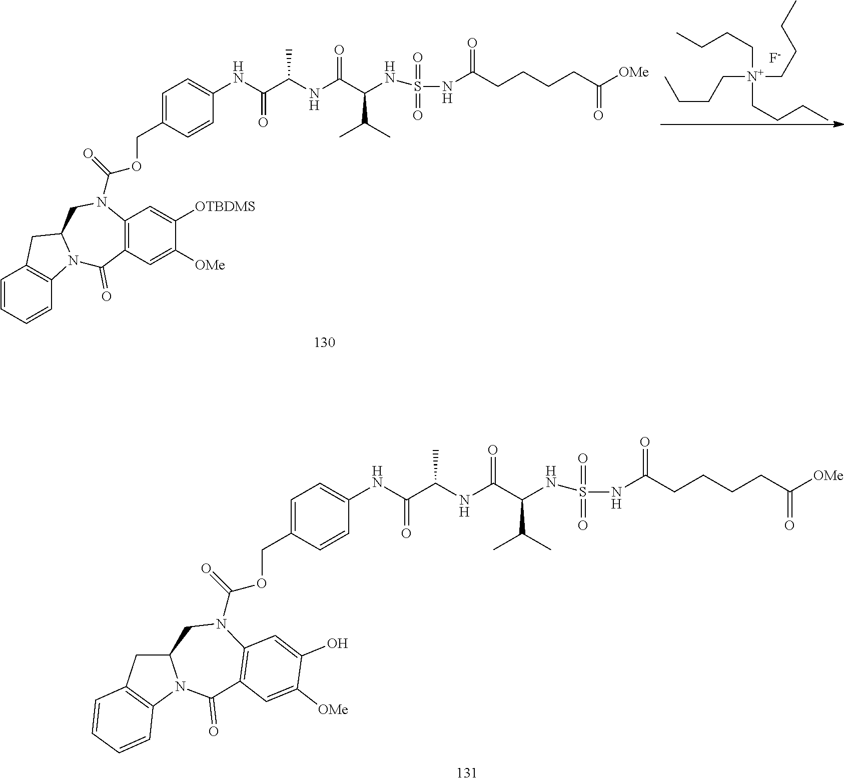

A solution of 4-((S)-2-((S)-2-((N-(6-methoxy-6-oxohexanoyl)sulfamoyl)amino)-3-methylbutanamido)propanamido)benzyl (S)-9-((tert-butyldimethylsilyl)oxy)-8-methoxy-6-oxo-12a,13-dihydro-6H-benzo[5,6][1,4]diazepino[1,2-a]indole-11(12H)-carboxylate (86 mg, 0.090 mmol) in anhydrous Tetrahydrofuran (904 μl) was cooled in an ice bath (0° C.) and tetrabutylammonium fluoride solution (1M in tetrahydrofuran, 181 μl, 0.181 mmol) was added. The mixture stirred for two hours at 0° C. under nitrogen. The reaction mixture was quenched with saturated aqueous ammonium chloride solution and extracted with ethyl acetate. The organic extracts were washed with brine, dried with anhydrous magnesium sulfate, filtered and concentrated. The crude material was purified by silica gel chromatographyI in methanol/dichloromethane) to yield 4-((S)-2-((S)-2-((N-(6-methoxy-6-oxohexanoyl)sulfamoyl)amino)-3-methylbutanamido)propanamido)benzyl (S)-9-hydroxy-8-methoxy-6-oxo-12a,13-dihydro-6H-benzo[5,6][1,4]diazepino[1,2-a]indole-11(12H)-carboxylate (59 mg, y=78%). MS (m/z): 837.8 (M+1)⁺835.7 (M−1)⁻. UPLC=1.54 min (2.5 min method).

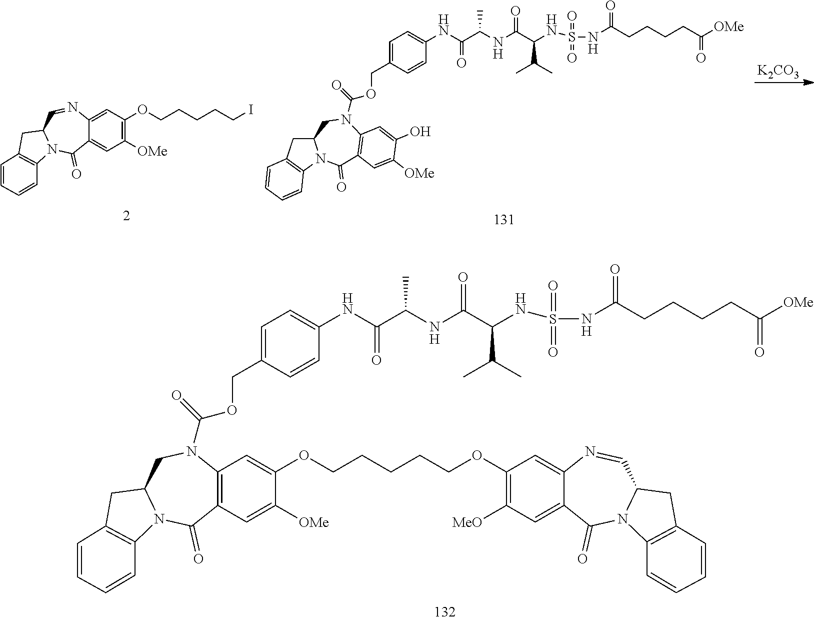

To a solution of (S)-9-((5-iodopentyl)oxy)-8-methoxy-12a,13-dihydro-6H-benzo[5,6][1,4]diazepino[1,2-a]indol-6-one (44.9 mg, 0.092 mmol) E003650-18 and 4-((S)-2-((S)-2-((N-(6-methoxy-6-oxohexanoyl)sulfamoyl)amino)-3-methylbutanamido)propanamido)benzyl (S)-9-hydroxy-8-methoxy-6-oxo-12a,13-dihydro-6H-benzo[5,6][1,4]diazepino[1,2-a]indole-11(12H)-carboxylate (59 mg, 0.070 mmol) in anhydrous N,N-Dimethylacetamide (705 μl) was added anhydrous potassium carbonate (19.49 mg, 0.141 mmol) and the reaction was stirred for 18 hours at room temperature. The reaction mixture was diluted with water and extracted with dichloromethane. The extracts were washed with water, dried over anhydrous magnesium sulfate and concentrated in vacuo. The crude material was purified by silica gel chromatography in methanol/dichloromethane to obtain compound 132 (42 mg, y=50%) as a white solid. MS (m/z): 1200.2 (M+1)⁺ UPLC=1.74 min (2.5 min method).

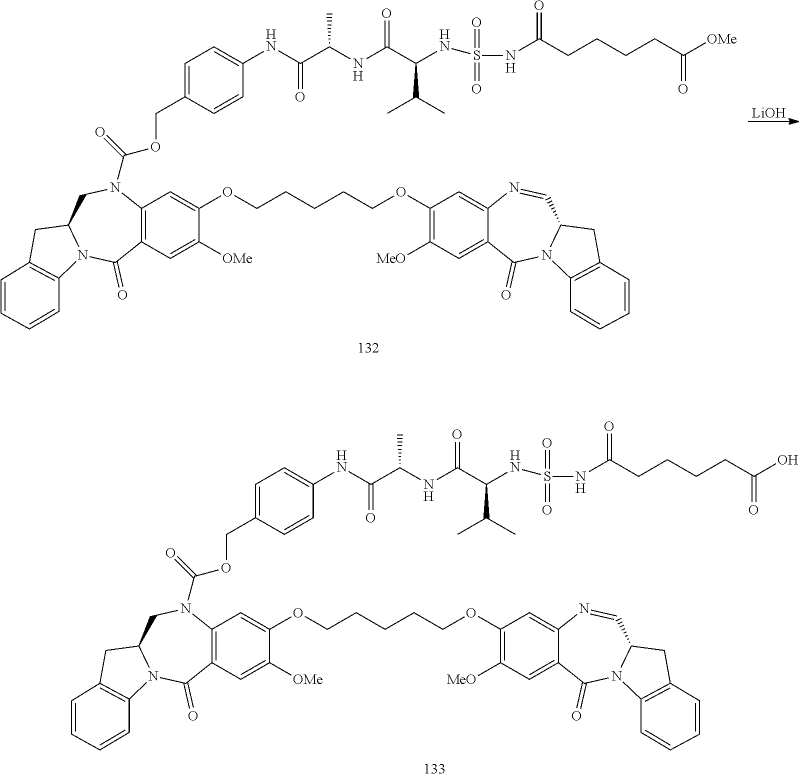 [SMILES: COC(=O)CCCCC(=O)NS(=O)(=O)N[C@H](C(=O)N[C@@H](C)C(=O)Nc1ccc(COC(=O)N2C[C@@H]3Cc4ccccc4N3C(=O)c3cc(OC)c(OCCCCCOc4cc5c(cc4OC)C(=O)N4c6ccccc6C[C@H]4C=N5)cc32)cc1)C(C)C ; "LiOH" ; COc1cc2c(cc1OCCCCCOc1cc3c(cc1OC)C(=O)N1c4ccccc4C[C@H]1CN3C(=O)OCc1ccc(NC(=O)[C@H](C)NC(=O)[C@@H](NS(=O)(=O)NC(=O)CCCCC(=O)O)C(C)C)cc1)N=C[C@@H]1Cc3ccccc3N1C2=O]

A solution of 4-((S)-2-((S)-2-((N-(6-methoxy-6-oxohexanoyl)sulfamoyl)amino)-3-methylbutanamido)propanamido)benzyl (S)-8-methoxy-9-((5-(((S)-8-methoxy-6-oxo-12a,13-dihydro-6H-benzo[5,6][1,4]diazepino[1,2-a]indol-9-yl)oxy)pentyl)oxy)-6-oxo-12a,13-dihydro-6H-benzo[5,6][1,4]diazepino[1,2-a]indole-11(12H)-carboxylate (28 mg, 0.023 mmol) in anhydrous Tetrahydrofuran (875 μl) and deionized water (292 μl) was cooled in an ice bath and lithium hydroxide (1.677 mg, 0.070 mmol) was added. The reaction stirred from 0° C. to room temperature for three hours, after which it was diluted with dichloromethane and deionized water. The mixture was acidified to pH=3 with hydrochloric acid (0.5 M aqueous, 1 mL) and extracted with 20% methanol in dichloromethane (2×˜20 mL). The organic layer was washed with water, dried over anhydrous magnesium sulfate, filtered and concentrated to obtain Compound 133 (17.6 mg, y=63%), which was used without further purification. MS (m/z): 1186.3 (M+1)⁺ UPLC=1.67 min (2.5 min method).

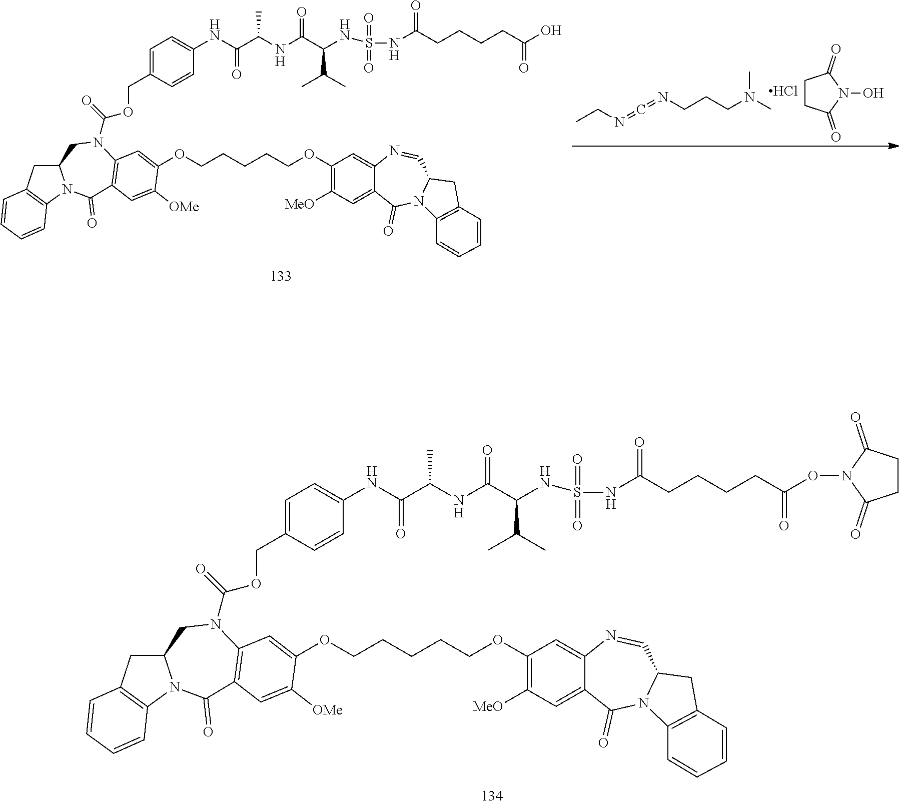

To a solution of 6-((N-((S)-1-(((S)-1-((4-((((S)-8-methoxy-9-((5-(((S)-8-methoxy-6-oxo-12a,13-dihydro-6H-benzo[5,6][1,4]diazepino[1,2-a]indol-9-yl)oxy)pentyl)oxy)-6-oxo-11,12,12a,13-tetrahydro-6H-benzo[5,6][1,4]diazepino[1,2-a]indole-11-carbonyl)oxy)methyl)phenyl)amino)-1-oxopropan-2-yl)amino)-3-methyl-1-oxobutan-2-yl)sulfamoyl)amino)-6-oxohexanoic acid (18 mg, 0.015 mmol) in anhydrous dichloromethane (607 μl) was added N-hydroxy succinimide (5.24 mg, 0.046 mmol) and 1-ethyl-3-(3-dimethylaminopropyl) carbodiimide hydrochloride (14.56 mg, 0.076 mmol). The reaction mixture stirred for 90 minutes at room temperature under nitrogen and was then diluted with dichloromethane and washed with water. The organic layer was dried with anhydrous magnesium sulfate, filtered and concentrated. The crude material was purified by RP-HPLC (C18 in acetonitrile/water, 50-70% over 30 min) Fractions containing product were combined, frozen and lyophilized to obtain compound 134 (5.3 mg, y=27%). MS (m/z): 1283.3 (M+1)⁺ UPLC=1.75 min (2.5 min method).

### Example 24. Synthesis of Compound 147

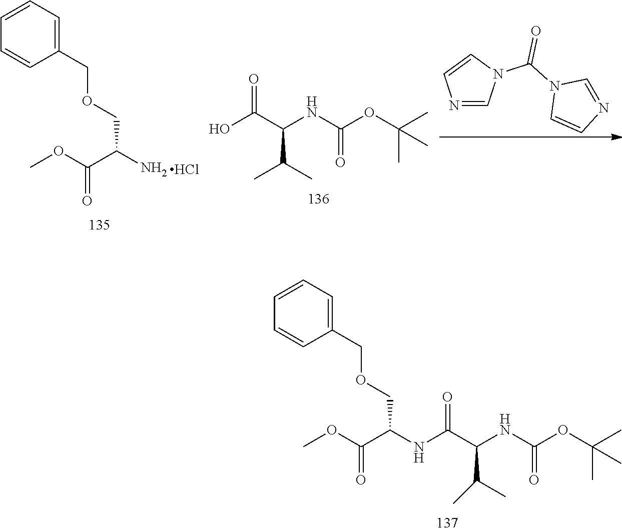

To a solution of (tert-butoxycarbonyl)-L-valine (8.5 g, 39.1 mmol) (Boc-Val-OH) in dichloromethane (78 ml) was added 1,1′-Carbonyldiimidazole (7.61 g, 46.9 mmol) was added in portions at room temperature. The reaction stirred at room temperature under nitrogen for 30 minutes. methyl O-benzyl-L-serinate hydrochloride (9.90 g, 40.3 mmol) ChemImpex in dichloromethane (19.56 ml) was added and the mixture was stirred under nitrogen for an additional 18 hours at room temperature. The reaction was diluted with dichlromethane, washed with hydrochloric acid (1 M aqueous), saturated aqueous sodium bicarbonate and brine. The organic layer was dried over anhydrous mag sulfate, filtered and concentrated to give methyl O-benzyl-N-((tert-butoxycarbonyl)-L-valyl)-L-serinate 17.8 g as a white solid. The crude was carried on without further purification, assuming 100% yield. MS (m/z): 409.6 (M+1)⁺ UPLC=1.60 min (2.5 min method).

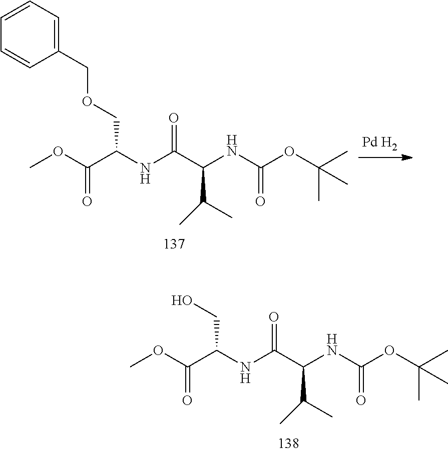

To a solution of methyl O-benzyl-N-((tert-butoxycarbonyl)-L-valyl)-L-serinate (12.7 g, 31.1 mmol) in anhydrous methanol (120 ml) was added dry palladium on carbon (10%, 1.654 g, 1.554 mmol). The black suspension was degassed and purged with hydrogen three times. The mixture was stirred under hydrogen balloon (1 atm) at room temperature for 18 hours after which it was filtered through Celite and rinsed with methanol to obtain methyl (tert-butoxycarbonyl)-L-valyl-L-serinate (10.6 g) as a sticky white solid. The crude was carried on without further purification, assuming 100% yield.

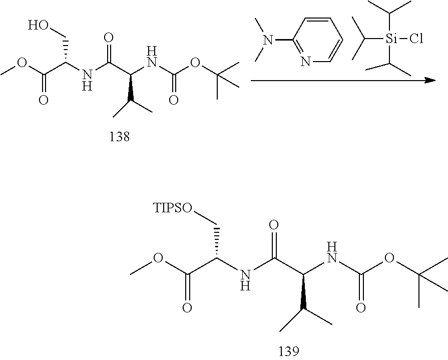

To a solution of methyl (tert-butoxycarbonyl)-L-valyl-L-serinate (4 g, 12.56 mmol) in anhydrous dimethylformamide (100 ml) was added Triisopropylsilyl chloride (4.16 ml, 18.85 mmol) and 4-Dimethylaminopyridine (4.60 g, 37.7 mmol). The mixture was stirred for 18 hours at room temperature under argon. Then diluted with ethyl acetate and washed with water and brine. The organic layer was dried over anhydrous magnesium sulfate, filtered and concentrated. The crude was purified by silica gel chromatography in ethyl acetate/hexanes to obtain methyl N-((tert-butoxycarbonyl)-L-valyl)-O-(triisopropylsilyl)-L-serinate (5.9 g, y=99%) as a white solid.

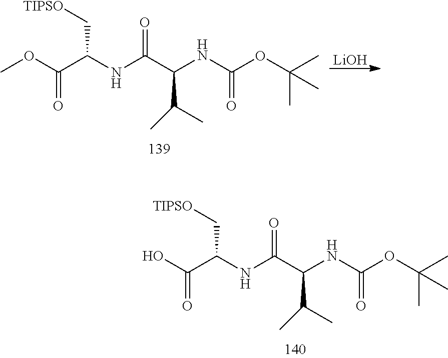

A solution of methyl N-((tert-butoxycarbonyl)-L-valyl)-O-(triisopropylsilyl)-L-serinate (5.9 g, 12.43 mmol) in Tetrahydrofuran (237 ml) and deionized water (118 ml) was cooled to 0° C. in an ice bath. Lithium hydroxide (0.893 g, 37.3 mmol) was added and the reaction was stirred for 2.5 hours at 0° C. The mixture was diluted with water and acidified with hydrochloric acid (1M aqueous). Then it was extracted with ethyl acetate. The extracts were washed with brine, dried with anhydrous magnesium sulfate, filtered and concentrated to obtain tert-butyl ((S)-1-(((S)-1-((4-(hydroxymethyl)phenyl)amino)-1-oxo-3-((triisopropylsilyl)oxy)propan-2-yl)amino)-3-methyl-1-oxobutan-2-yl)carbamate (5.5 g, 96%) as a white crystalline solid which was used without further purification.

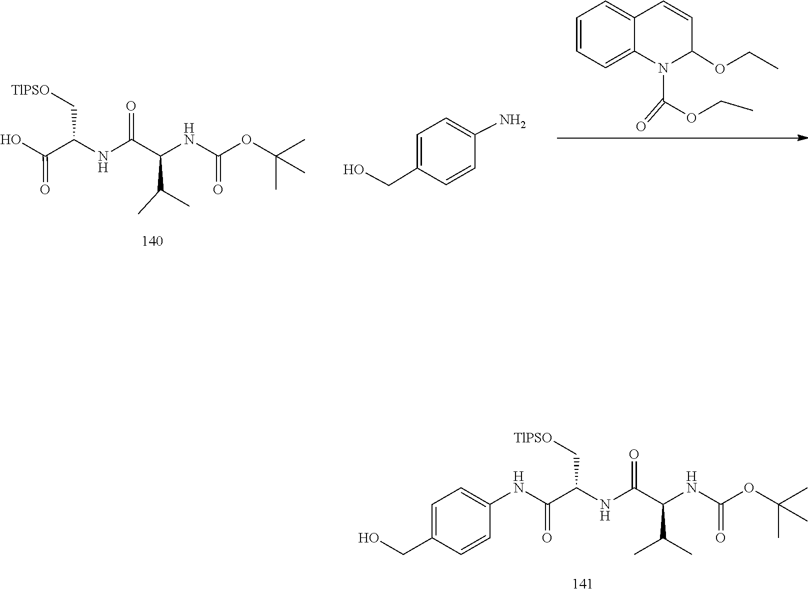

A solution of tert-butyl ((S)-1-(((S)-1-((4-(hydroxymethyl)phenyl)amino)-1-oxo-3-((triisopropylsilyl)oxy)propan-2-yl)amino)-3-methyl-1-oxobutan-2-yl)carbamate and EEDQ (5.62 g, 22.74 mmol) in anhydrous dichloromethane (196 ml) were stirred at room temperature for one hour. Then (4-aminophenyl)methanol (1.4 g, 11.37 mmol) was added and the mixture stirred for an additional 18 hours at room temperature. The reaction was concentrated to dryness, then redissolved in dichlromethane and filtered through celite. The filtrate was evaporated and purified by silica gel chromatography in ethyl acetate/hexanes to obtain tert-butyl ((S)-1-(((S)-1-((4-(hydroxymethyl)phenyl)amino)-1-oxo-3-((triisopropylsilyl)oxy)propan-2-yl)amino)-3-methyl-1-oxobutan-2-yl)carbamate (1.47 g, y=23%) as a crystalline white solid. MS (m/z): 566.7 (M+1)⁺564.7 (M−1) UPLC=1.92 min (2.5 min method).

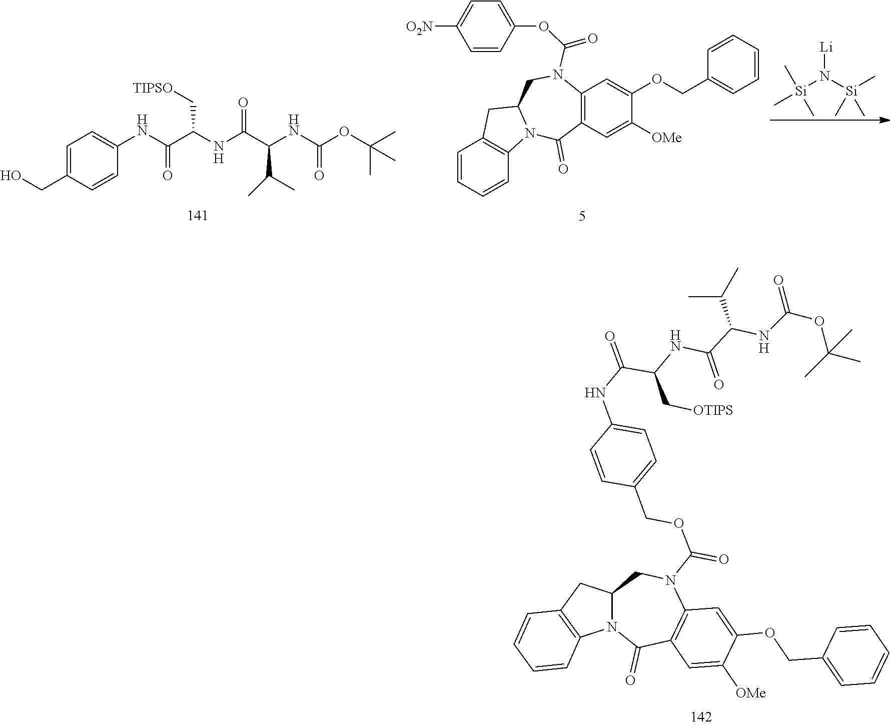

A solution of tert-butyl ((S)-1-(((S)-1-((4-(hydroxymethyl)phenyl)amino)-1-oxo-3-((triisopropylsilyl)oxy)propan-2-yl)amino)-3-methyl-1-oxobutan-2-yl)carbamate (1.436 g, 2.54 mmol) in anhydrous tetrahydrofuran (5.77 ml) and anhydrous N,N-dimethylacetamide (11.54 ml) was cooled to 0° C. in an ice bath. Lithium bis(trimethylsilyl)amide (1M in tetrahydrofuran, 3.05 ml, 3.05 mmol) was added and the mixture stirred for 20 minutes under argon then 4-nitrophenyl (S)-9-(benzyloxy)-8-methoxy-6-oxo-12a,13-dihydro-6H-benzo[5,6][1,4]diazepino[1,2-a]indole-11(12H)-carboxylate (1.4 g, 2.54 mmol) in anhydrous tetrahydrofuran (5.77 ml) was added. The reaction mixture was stirred from 0° C. to room for 18 hours under argon, then quenched with saturated ammonium chloride solution. The mixture was extracted with ethyl acetate and the organic layer was washed with water and brine. The organic layer was dried with anhydrous magnesium sulfate, filtered and concentrated. The crude material was purified by silica gel chromatography in ethyl acetate/dichloromethane to obtain 4-((S)-2-((S)-2-((tert-butoxycarbonyl)amino)-3-methylbutanamido)-3-((triisopropylsilyl)oxy)propanamido)benzyl (S)-9-(benzyloxy)-8-methoxy-6-oxo-12a,13-dihydro-6H-benzo[5,6][1,4]diazepino[1,2-a]indole-11(12H)-carboxylate (0.91 g, y=36%) as a white solid. MS (m/z): 979.2 (M+1)⁺ UPLC=2.24 min (2.5 min method).

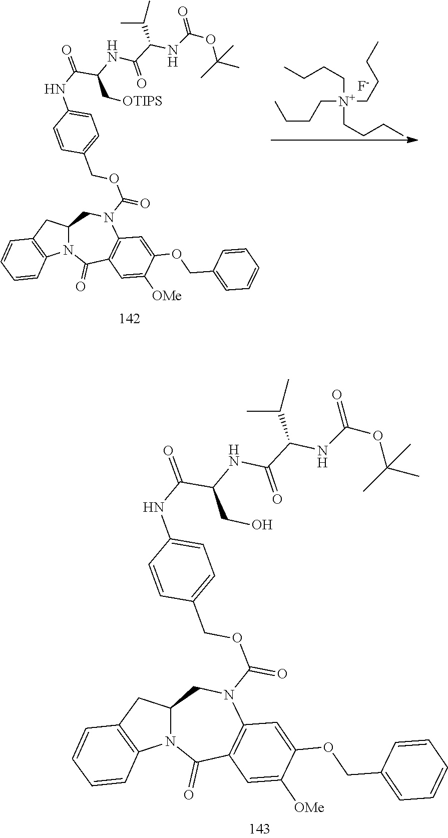

A solution of 4-((S)-2-((S)-2-((tert-butoxycarbonyl)amino)-3-methylbutanamido)-3-((triisopropylsilyl)oxy)propanamido)benzyl (S)-9-(benzyloxy)-8-methoxy-6-oxo-12a,13-dihydro-6H-benzo[5,6][1,4]diazepino[1,2-a]indole-11(12H)-carboxylate (0.5 g, 0.511 mmol) in anhydrous Tetrahydrofuran (5.11 ml) was cooled to 0° C. in an ice bath and tetrabutylammonium fluoride solution (1M in Tetrahydrofuran, 1.022 ml, 1.022 mmol) was added. The reaction mixture stirred at 0° C. for two hours under argon and upon completion was quenched with saturated ammonium chloride solution. The mixture was extracted with ethyl acetate. The extracts were washed with brine, dried with anhydrous magnesium sulfate, filtered and concentrated. The crude material was purified by silica gel chromatography in ethyl acetate/dichloromethane to obtain compound 143 (356 mg, y=85%) as a white solid. MS (m/z): 822.9 (M+1)⁺ UPLC=1.82 min (2.5 min method).

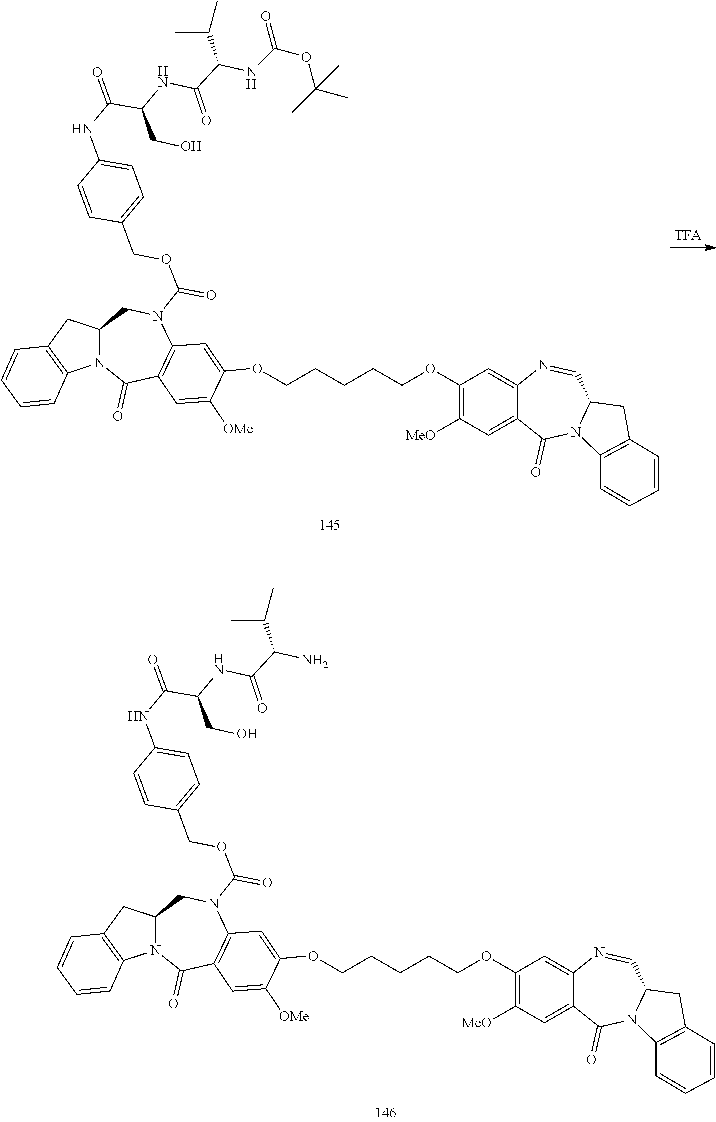 [SMILES: COc1cc2c(cc1OCCCCCOc1cc3c(cc1OC)C(=O)N1c4ccccc4C[C@H]1CN3C(=O)OCc1ccc(NC(=O)[C@H](CO)NC(=O)[C@@H](NC(=O)OC(C)(C)C)C(C)C)cc1)N=C[C@@H]1Cc3ccccc3N1C2=O ; COc1cc2c(cc1OCCCCCOc1cc3c(cc1OC)C(=O)N1c4ccccc4C[C@H]1CN3C(=O)OCc1ccc(NC(=O)[C@H](CO)NC(=O)[C@@H](N)C(C)C)cc1)N=C[C@@H]1Cc3ccccc3N1C2=O]

A solution of 4-((S)-2-((S)-2-((tert-butoxycarbonyl)amino)-3-methylbutanamido)-3-hydroxypropanamido)benzyl (S)-8-methoxy-9-((5-(((S)-8-methoxy-6-oxo-12a,13-dihydro-6H-benzo[5,6][1,4]diazepino[1,2-a]indol-9-yl)oxy)pentyl)oxy)-6-oxo-12a,13-dihydro-6H-benzo[5,6][1,4]diazepino[1,2-a]indole-11(12H)-carboxylate (90 mg, 0.082 mmol) in anhydrous dichloromethane (0.8 ml) was cooled to 0° C. in an ice bath. A fresh mixture of Trifluoroacetic acid (411 μl) in anhydrous dichloromethane (0.4 ml) was added. The reaction stirred for one hour at 0° C. under argon and upon completion the mixture was poured into an ice/saturated sodium bicarbonate mixture. This mixture was extracted with dichloromethane, washed with brine and dried with anhydrous magnesium sulfate, filtered and concentrated to get compound 146 (63 mg, y=77%) as a yellow solid. MS (m/z): 995.2 (M+1)⁺ UPLC=1.51 min (2.5 min method).

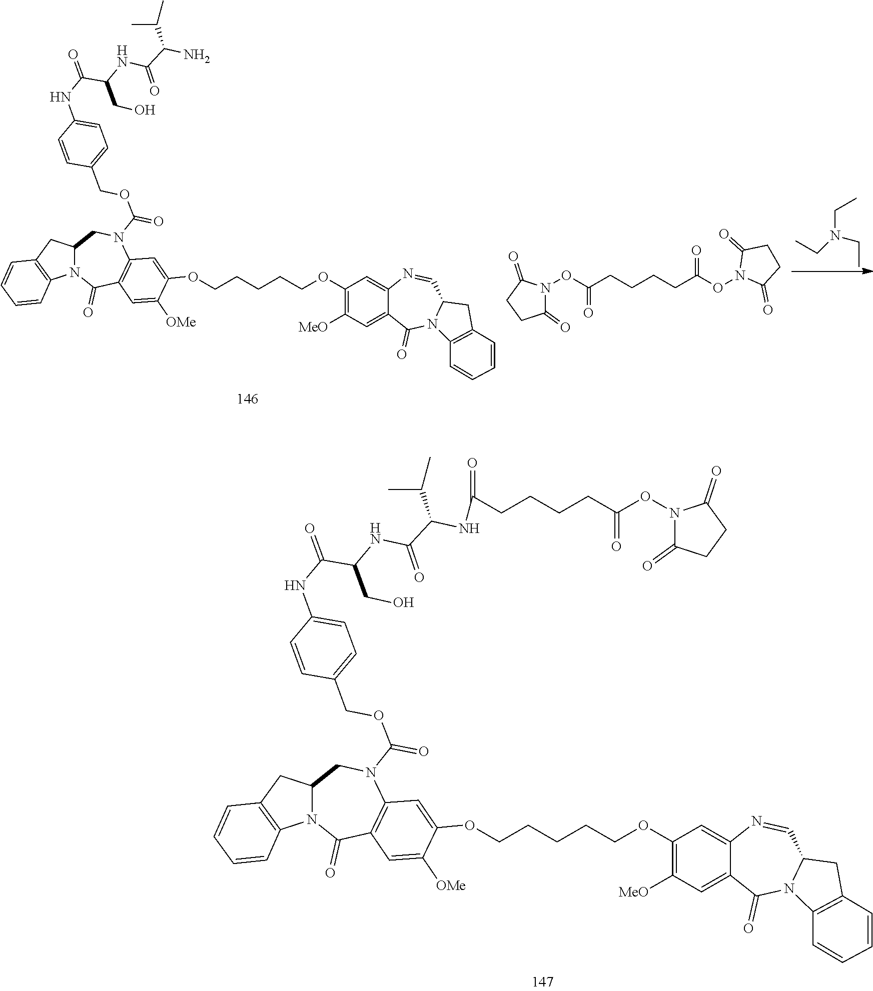

To a solution of 4-((S)-2-((S)-2-amino-3-methylbutanamido)-3-hydroxypropanamido)benzyl (S)-8-methoxy-9-((5-(((S)-8-methoxy-6-oxo-12a,13-dihydro-6H-benzo[5,6][1,4]diazepino[1,2-a]indol-9-yl)oxy)pentyl)oxy)-6-oxo-12a,13-dihydro-6H-benzo[5,6][1,4]diazepino[1,2-a]indole-11(12H)-carboxylate (36 mg, 0.036 mmol) in anhydrous N,N-dimethylformamide (724 μl) was added bis(2,5-dioxopyrrolidin-1-yl) adipate (24.65 mg, 0.072 mmol) and Triethylamine (15.14 μl, 0.109 mmol) were added. The reaction stirred at room temperature under argon for one hour and was then diluted with dichloromethane and washed with water. The organic layer was dried, filtered and coevaporated with acetonitrile. The crude material was purified by RP-HPLC (C18 in acetonitrile/water, 50-70% over 30 min) to give compound 147. MS (m/z): 1220.4 (M+1)+ UPLC=1.75 min (2.5 min method).

### Example 25. Synthesis of Compound 148

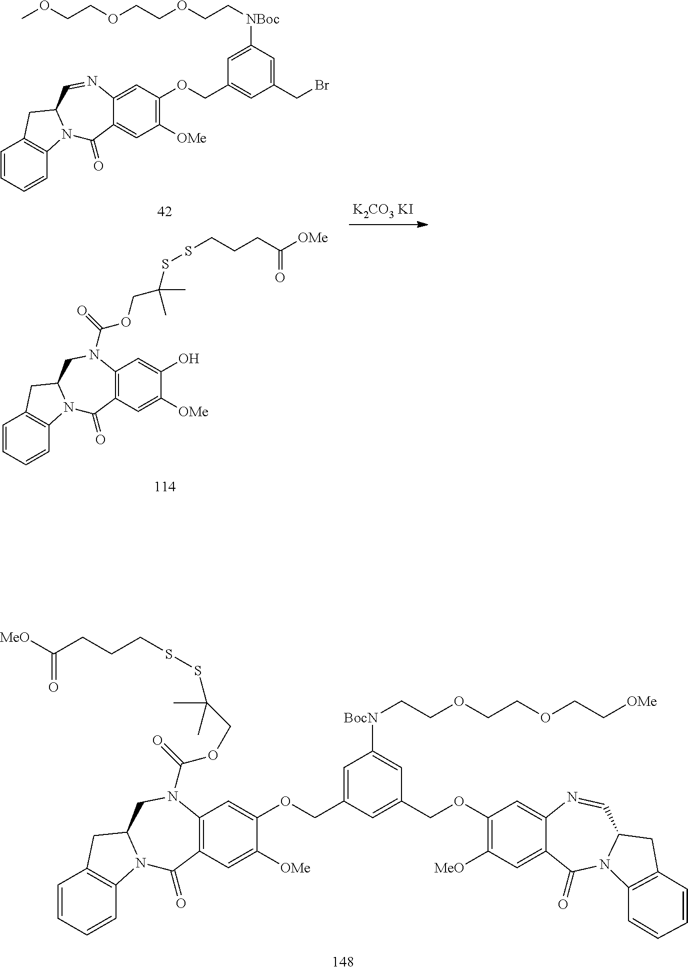 [SMILES: COCCOCCOCCN(C(=O)OC(C)(C)C)c1cc(CBr)cc(COc2cc3c(cc2OC)C(=O)N2c4ccccc4C[C@H]2C=N3)c1 ; COC(=O)CCCSSC(C)(C)COC(=O)N1C[C@@H]2Cc3ccccc3N2C(=O)c2cc(OC)c(O)cc21 ; "K2CO3 KI" ; COCCOCCOCCN(C(=O)OC(C)(C)C)c1cc(COc2cc3c(cc2OC)C(=O)N2c4ccccc4C[C@H]2C=N3)cc(COc2cc3c(cc2OC)C(=O)N2c4ccccc4C[C@H]2CN3C(=O)OCC(C)(C)SSCCCC(=O)OC)c1]

To a suspension of tert-butyl (S)-(3-(bromomethyl)-5-(((8-methoxy-6-oxo-12a,13-dihydro-6H-benzo[5,6][1,4]diazepino [1,2-a]indol-9-yl)oxy)methyl)phenyl)(2-(2-(2-methoxyethoxy)ethoxy)ethyl)carbamate (119 mg, 0.161 mmol) and 2-((4-methoxy-4-oxobutyl)disulfanyl)-2-methylpropyl (S)-9-hydroxy-8-methoxy-6-oxo-12a,13-dihydro-6H-benzo[5,6][1,4]diazepino[1,2-a]indole-11(12H)-carboxylate (75 mg, 0.134 mmol) in anhydrous N,N-dimethylacetamide (1.34 ml) was added potassium carbonate (37.0 mg, 0.268 mmol) and the reaction was stirred for 18 hours at room temperature under nitrogen. water was added to the reaction and the resulting white solid was filtered, then re-dissolved in dichloromethane, transferred to a separatory funnel, washed with water, dried over anhydrous magnesium sulfate and concentrated in vacuo. Purified the crude material via RP-HPLC (C18 Kromasil, acetonitrile/water). Pure fractions were extracted with dichloromethane. Dried with mag sulfate, filtered and concentrated to give compound 148.

### Example 26. Synthesis of Compound 150

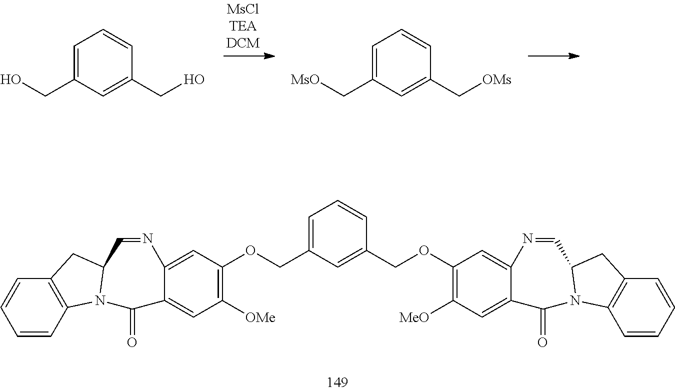 [SMILES: OCc1cccc(CO)c1 ; CS(=O)(=O)OCc1cccc(COS(C)(=O)=O)c1 ; COc1cc2c(cc1OCc1cccc(COc3cc4c(cc3OC)C(=O)N3c5ccccc5C[C@H]3C=N4)c1)N=C[C@@H]1Cc3ccccc3N1C2=O]

To a stirred solution of 1,3-benzenedimethanol (11 mg, 0.08 mmol) in anhydrous dichloromethane (0.8 mL) was added triethylamine (33 μl, 0.24 mmol) then methanesulfonyl chloride (16 μL, 0.21 mmol) dropwise in 15 minutes at −5˜−10° C. The solution was stirred at −5˜−10° C. for another 60 minutes and was quenched with ice/water, diluted with cold ethyl acetate. The mixture was separated and the organic layer was washed with cold water, dried over anhydrous sodium sulfate. It was filtered and the filtrate was evaporated by rotary evaporation in vacuo (temperature <35° C.). The dimesylate obtained was high vacuumed for a few hours before being dissolved in anhydrous dimethylformamide (1.5 mL). Compound 1 (94 mg, 0.32 mmol), anhydrous potassium carbonate (50 mg, 0.36 mmol) and potassium iodide (27 mg, 0.16 mmol) were added subsequently. The mixture was stirred at room temperature for 17 hours (checked by mass spectrum) and diluted with dichloromethane. It was washed with brine, dried over anhydrous sodium sulfate and filtered. The filtrate was evaporated under reduced pressure and the residue was purified by reverse phase HPLC (C18 column, acetonitrile/H₂O, loaded column with acetonitrile/H₂O, 3:1, stirred for 30 min and centrifuged before injection) to furnish dimer compound 149 (6.6 mg) as a white solid. ¹H NMR (400 Hz, CDCl₃): δ 8.21 (d, J=8.0 Hz, 2H), 7.79 (d, J=4.4 Hz, 2H), 7.51 (s, 2H), 7.46 (s, 1H), 7.36 (bs, 3H), 7.23-7.18 (m, 4H), 7.06-7.03 (m, 2H), 6.79 (s, 2H), 5.20 (d, J=12.4 Hz, 2H), 5.14 (d, J=12.4 Hz, 2H), 4.41 (ddd, J1=10.8 Hz, J2=4.4 Hz, J3=4.0 Hz, 2H), 3.92 (s, 6H), 3.64 (dd, J1=17.2 Hz, J2=11.2 Hz, 2H), 3.42 (dd, J1=16.8 Hz, J2=4.0 Hz, 2H); HRMS (ESI, m/z): calc. 691.2557 (M+H)⁺, found 691.2570.

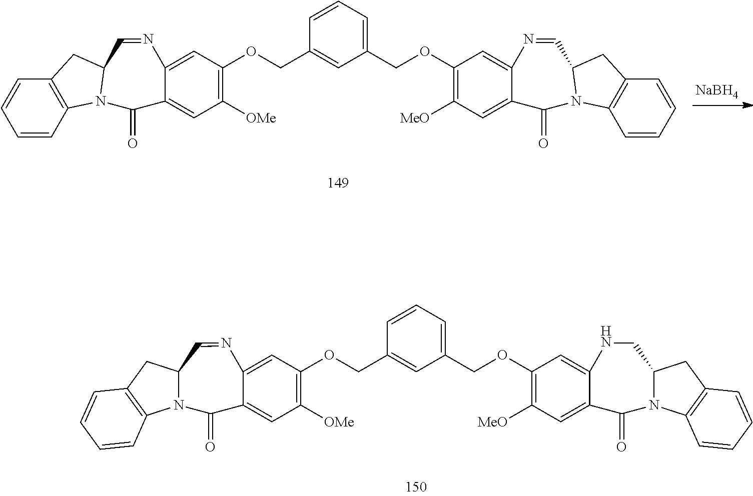

Compound 149 (60 mg, 0.043 mmol) was dissolved in an anhydrous mixture of dichloromethane (0.25 ml) and ethanol (0.5 ml) and cooled to 0° C. in an ice bath. A sodium borohydride (0.493 mg, 0.013 mmol) solution dissolved in ethanol (50 ul) was then added and the mixture was stirred for 5 minutes and the ice bath was removed. The reaction was allowed to stir for 3 hours, quenched at low temperature by adding saturated ammonium chloride and dichloromethane, separated and the organic layer was washed with brine, dried over anhydrous sodium sulfate, filtered, and concentrated in vacuo. The residue was purified by semi-prep RP-HPLC (C18 column, acetonitrile/H₂O and the fractions containing the desired products were extracted with dichloromethane and concentrated to give the mono-imine compound 150 (20 mg, 33%) MS (m/z), expected: 692.7, found: 715.2 (M+Na)⁺, 733.2 (M+H₂O+Na)⁺, 749.2 (M+H₂O+K)⁺.

### Example 27. Synthesis of Compound 155

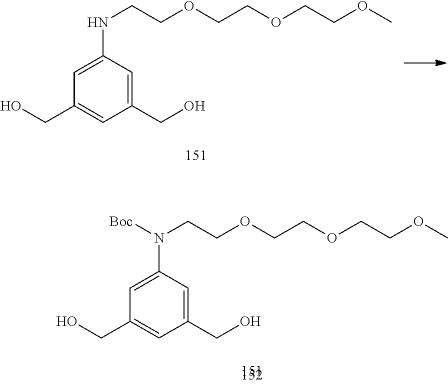 [SMILES: COCCOCCOCCNc1cc(CO)cc(CO)c1 ; COCCOCCOCCN(C(=O)OC(C)(C)C)c1cc(CO)cc(CO)c1]

To a stirred solution of the compound 151 (339 mg, 1.1 mmol) in anhydrous tetrahydrofuran (4.0 mL) was added Boc anhydride (272 mg, 1.2 mmol). The mixture was continued to be stirred at room temperature for three days. The reaction mixture was concentrated under reduced pressure and the residue was purified by silica gel chromatography (dichloromethane/methanol) to give compound 152 (405 mg, y=90%) as colorless oil. 1H NMR (400 Hz, CDCl₃): δ 7.00 (s, 2H), 6.97 (s, 1H), 4.38 (s, 4H), 4.12 (s, 2h), 3.64 (t, J=5.6 Hz, 2H), 3.48-3.44 (m, 8H), 3.40-3.38 (m, 2H), 3.21 (s, 3H), 1.31 (s, 9H); ¹³C NMR (400 Hz, CDCl₃): δ 154.65, 142.3, 142.1, 124.1, 122.7, 80.2, 71.6, 70.3, 70.1, 69.9, 68.5, 63.9, 58.65, 49.4, 28.1.

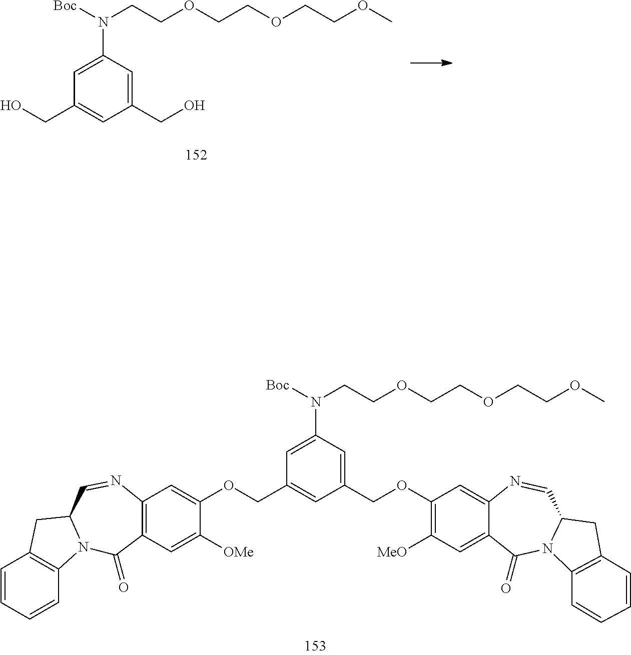 [SMILES: COCCOCCOCCN(C(=O)OC(C)(C)C)c1cc(CO)cc(CO)c1 ; COCCOCCOCCN(C(=O)OC(C)(C)C)c1cc(COc2cc3c(cc2OC)C(=O)N2c4ccccc4C[C@H]2C=N3)cc(COc2cc3c(cc2OC)C(=O)N2c4ccccc4C[C@H]2C=N3)c1]

To a stirred solution of compound 152 (51 mg, 0.128 mmol) in anhydrous dichloromethane was added triethylamine (0.053 mL, 0.383 mmol) at −5-10° C. Methanesulfonyl chloride (0.026 mL, 0.332 mmol) was then added slowly in 15 minutes with a syringe. The mixture was stirred at ˜5-10° C. for 1 hours (TLC, dichloromethane/methanol 10:1). The reaction was quenched with ice/water, diluted with cold AcOEt, separated and the organic layer was washed with cold water, dried over anhydrous Na₂SO₄/MgSO₄, filtered and stripped. The residue was transferred into a small reaction flask with dichloromethane, stripped and high vacuumed. It was dissolved in anhydrous dimethylformamide (0.8 mL) followed by addition of compound 1 (90 mg, 0.31 mmol) and potassium (53 mg, 0.38 mmol). The mixture was stirred at room temperature overnight. It was diluted with dichloromethane, washed with brine, dried over anhydrous sodium sulfate, filtered and stripped. The residue was purified by reverse phase HPLC (CI 8, acetonitrile/water) to give compound 32b (56 mg, 46%) as yellowish solid. %). 1H NMR (400 Hz, CDCl₃): δ 8.29 (d, J=8.0 Hz, 2H), 7.87 (d, J=4.8 Hz, 2H), 7.60 (s, 2H), 7.38-7.36 (m, 3H), 7.33-7.27 (m, 4H), 7.13 (t, J=7.6 Hz, 2H), 6.88 (s, 2H), 5.21 (dd, =20.0 Hz, J₂=12.4 Hz, 4H), 4.49 (dt, Jj=11.2 Hz, J₂=4.0 Hz, 2H), 3.99 (s, 6H), 3.83 (t, J=6.0 Hz, 2H), 3.76-3.48 (m, 14H), 3.35 (s, 3H), 1.43 (s, 9H); MS (m/z): found 992.2 (M+H₂O+Na)⁺, 101+.

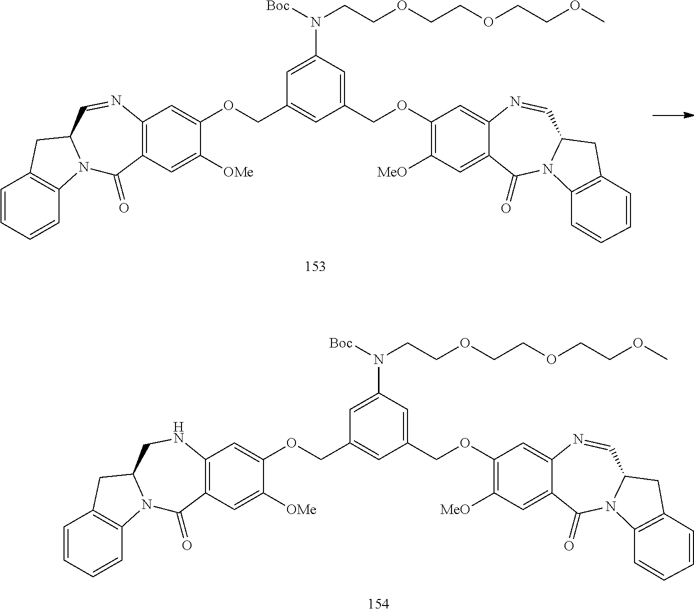 [SMILES: COCCOCCOCCN(C(=O)OC(C)(C)C)c1cc(COc2cc3c(cc2OC)C(=O)N2c4ccccc4C[C@H]2C=N3)cc(COc2cc3c(cc2OC)C(=O)N2c4ccccc4C[C@H]2C=N3)c1 ; COCCOCCOCCN(C(=O)OC(C)(C)C)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

To a stirred solution of compound 153 (56 mg, 0.059 mmol) in anhydrous dichloromethane (0.3 mL) and absolute ethanol (0.9 mL) was added NaBH₄ (2.7 mg, 0.07 mmol) at 0° C. The ice bath was removed and the mixture was stirred at room temperature for 3 hours and then quenched with saturated ammonium chloride, diluted with dichloromethane, separated and the organic layer was washed with brine, dried over anhydrous Na₂SO₄ and filtered through celite and stripped. All the fractions that contained pure product were extracted with dichloromethane and stripped to give compound 154 (20.7 mg, y=37%) as a light yellowish solid. MS (m/z): found 954.2 (M+H)⁺.

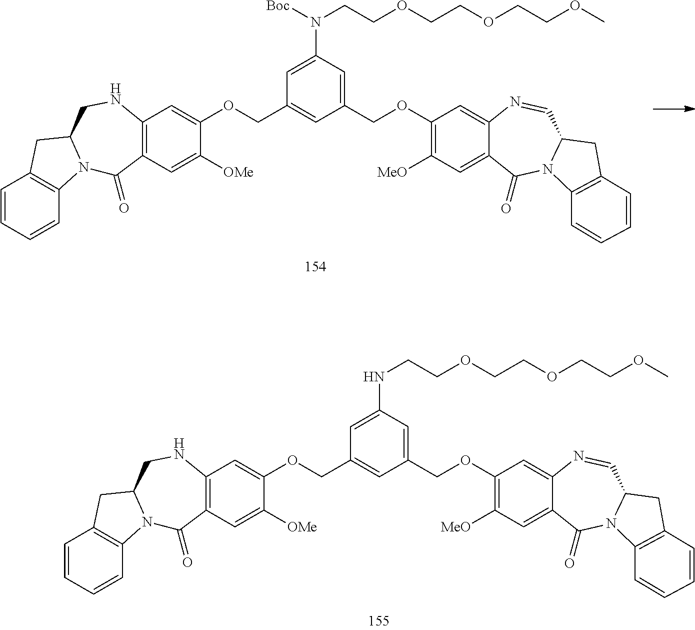 [SMILES: COCCOCCOCCN(C(=O)OC(C)(C)C)c1cc(COc2cc3c(cc2OC)C(=O)N2c4ccccc4C[C@H]2C=N3)cc(COc2cc3c(cc2OC)C(=O)N2c4ccccc4C[C@H]2CN3)c1 ; COCCOCCOCCNc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

Tert-butyl (3-((((S)-8-methoxy-6-oxo-11,12,12a,13-tetrahydro-6H-benzo[5,6][1,4]diazepino[1,2-a]indol-9-yl)oxy)methyl)-5-((((S)-8-methoxy-6-oxo-12a,13-dihydro-6H-benzo[5,6][1,4]diazepino[1,2-a]indol-9-yl)oxy)methyl)phenyl)(2-(2-(2-methoxyethoxy)ethoxy)ethyl)carbamate (273 mg, 0.286 mmol) was dissolved in 2.65 ml anhydrous dichloromethane and cooled to OC in an ice bath. A freshly mixed solution of 2.65 ml anhydrous dichloromethane and trifluroacetic acid (2649 μl) was added. The reaction stirred under argon at OC for 55 minutes. It was diluted with dichloromethane, poured into ice/saturated sodium bicarbonate. The separated organic was washed with brine and dried over magnesium sulfate, filtered and stripped to give 210 mgs of a pale yellow solid. Removed 46 mgs and purified by RPHPLC (water/acetronitrile). The fractions containing the desired compound were frozen and lyophilized to give final compound 155 (32 mg, y=60%). MS (m/z): 854.8 (M+1)⁺. UPLC=1.65 (2.5 min method).

### Example 28. Synthesis of Compound 157

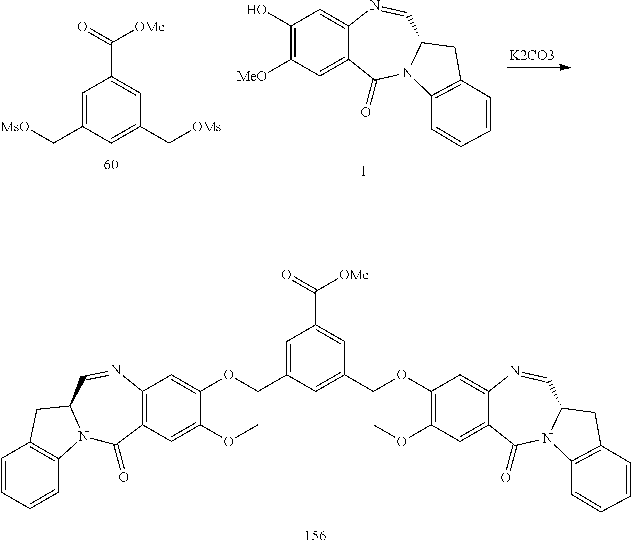

Compound 60 (180 mg, 0.511 mmol) and compound 1 (336 mg, 1.073 mmol) were dissolved in dimethylformamide (2554 μl). Potassium carbonate (176 mg, 1.277 mmol) was added at room temperature causing a color change to bright orange. The reaction was stirred under Ar overnight. The reaction was diluted with dichloromethane and washed with water (2×). The organic was dried, concentrated, and purified by silica gel chromatography (ethyl acetate/hexanes and then 5% methanol/dichloromethane). The pure product was collected to give compound 156 (300 mg, y=78%). MS (m/z): 849.5 (M+1)⁺. LCMS=8.2 (15 min method).

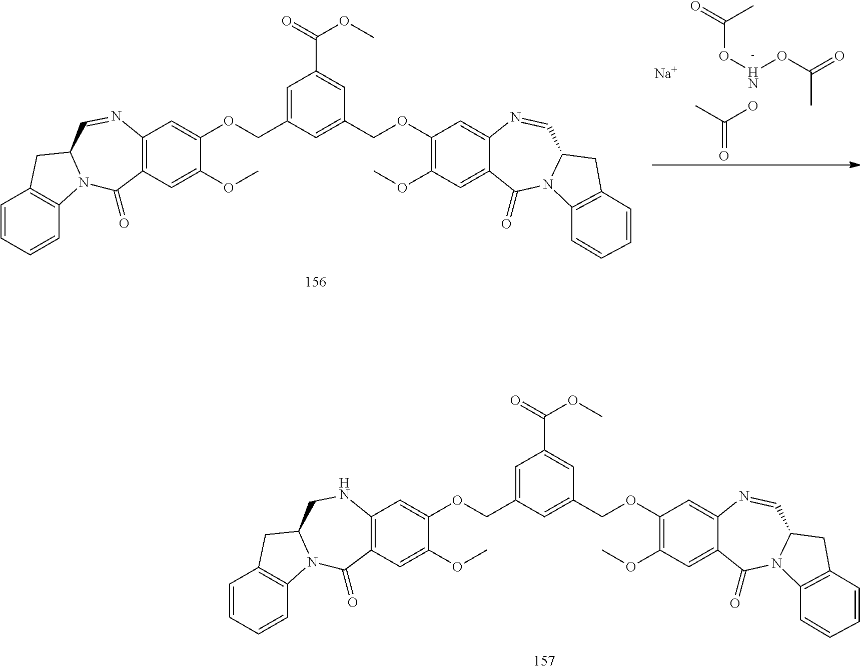

Compound 156 (45 mg, 0.060 mmol) was dissolved in 1, 2-Dichloroethane (601 μl). Sodium Triacetoxyborohydride (11.46 mg, 0.054 mmol) was added at room temperature and stirred for 1 hour. Added another ˜2 mg of STAB and stirred for 15 minutes. The reaction was diluted with ethyl acetate and a few drops of methanol and quenched with an aqueous citric acid solution. The layers were separated and the organic was washed with brine, dried and concentrated. The crude solid was diluted in dimethylformamide/acetonitrile/water/formic acid and purified by reverse phase C18 HPLC. The pure fractions containing the mono reduced product were frozen and lyophilized to give compound 157 as the desired product (10 mg, y=21%). MS (m/z): 751.7 (M+1)⁺. LCMS=5.8 (8 min method).

### Example 29. Synthesis of Compound 159

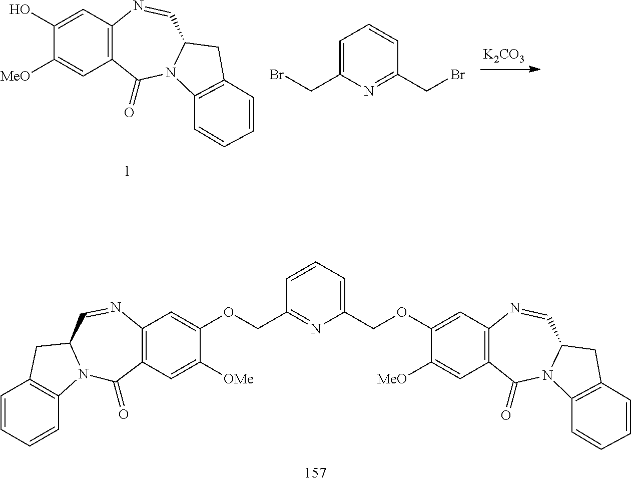

Compound 1 (120 mg, 0.407 mmol) and 2,6-bis(bromomethyl)pyridine (50 mg, 0.185 mmol) were dissolved in anhydrous dimethylformamide (1233 μl) and potassium carbonate (77 mg, 0.555 mmol) was added. The reaction stirred at room temperature for 5 hours and was complete. Water was added to precipitate product. The resulting solid was filtered and washed with water. The solid was redissolved in dichloromethane/methanol. The organic was dried over magnesium sulfate, filtered, and concentrated to give 163 mgs of crude compound 158. The crude material was carried on without further purification, assuming 100% yield. MS (m/z): 692.5 (M+1)⁺. UPLC=1.68 (2.5 min method).

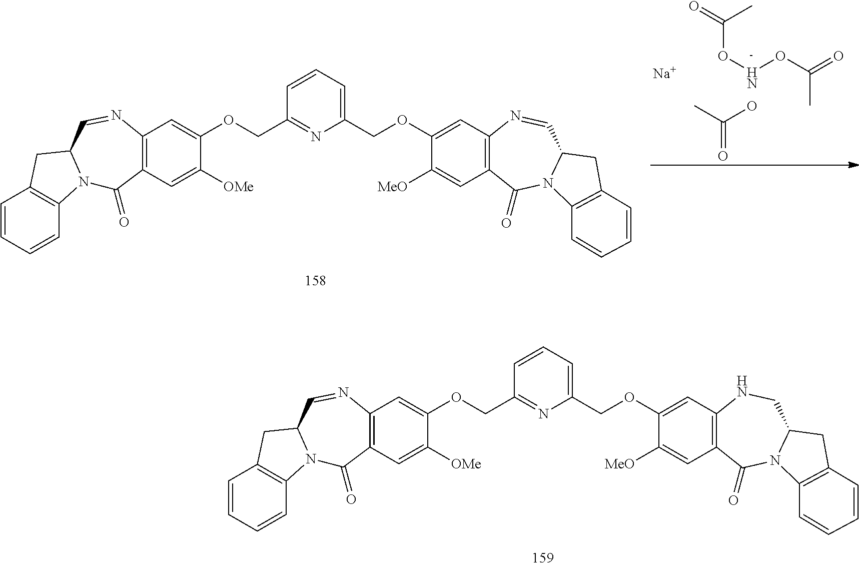

(12aS,12a′S)-9,9′-((pyridine-2,6-diylbis(methylene))bis(oxy))bis(8-methoxy-12a,13-dihydro-6H-benzo[5,6][1,4]diazepino[1,2-a]indol-6-one) (128 mg, 0.185 mmol) was dissolved in 1,2-dichloroethane (1850 μl) and sodium triacetoxyborohydride (39.2 mg, 0.185 mmol) was added at room temperature. The reaction was checked at 45 minutes. Added another 15 mgs of STAB and let stir 30 minutes. The reaction was diluted with dichloromethane and quenched with saturated ammonium chloride. The organic was washed with brine and dried over magnesium sulfate, filtered and stripped to give 165 mg of crude material. About half of the crude material was dissolved in tetrahydrofuran/acetonitrile/water and purified by RPHPLC (water/acetonitrile). The fractions containing the mono reduced product were frozen and lyophilized to give the desired compound 159 (19.5 mg, y=30%). MS (m/z): 694.6 (M+1)⁺. UPLC=1.77 (2.5 min method).

### Example 30. Synthesis of Compound 161

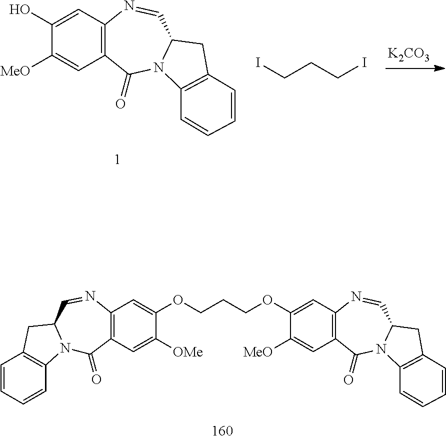

To a solution of compound 1 (147 mg, 0.5 mmol) and 1,3-diiodopropane (23 ul, 0.2 mmol) in anhydrous dimethylformamide (1.0 mL) was added potassium carbonate (111 mg, 0.8 mmol). The mixture was stirred at room temperature overnight (16 hours) and diluted with dichloromethane. It was washed with saturated ammonium chloride and brine, dried over anhydrous sodium sulfate and filtered. The filtrate was evaporated under reduced pressure and the residue was purified through preparative reverse phase HPLC (C18 column, acetonitrile/water) to give compound 160 (18.9 mg, 15%) as a white solid. ¹H NMR (400 Hz, CDCl₃): δ 8.26 (d, J=8.0 Hz, 2H), 7.87 (d, J=4.4 Hz, 2H), 7.55 (s, 2H), 7.26 (s, 4H), 7.12-7.08 (m, 2H), 6.88 (s, 2H), 4.45 (ddd, J₁=10.8 Hz, J₂=4.4 Hz, J₃=4.0 Hz, 2H), 4.36-4.26 (m, 4H), 3.94 (s, 6H), 3.70 (dd, J₁=16.8 Hz, J₂=10.8 Hz, 2H), 3.50 (dd, J₁=16.8 Hz, J₂=4.0 Hz, 2H), 2.45 (p, J=6.0 Hz, 2H); HRMS (ESI, m/z): calc. 629.2400 (M+H)⁺, found 629.2400.

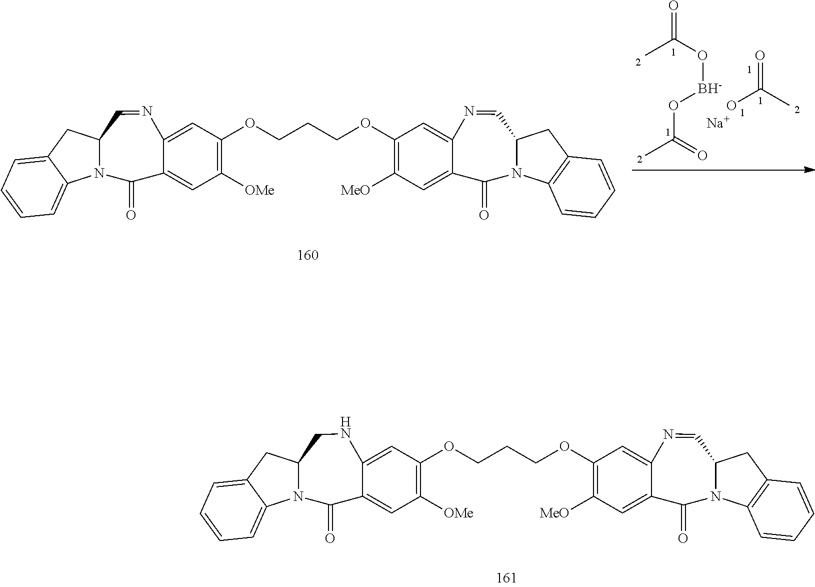

Procedure: To a stirred solution of compound 160 (331 mg, 0.527 mmol) in anhydrous 1,2-dichloroethane (3.5 ml) was added sodium triacetoxyborohydride (117 mg, 0.527 mmol) and the mixture was stirred at room temperature for 90 minutes under nitrogen. Quench the reaction with methanol (˜1 mL) and saturated sodium bicarbonate and diluted with DCM, separated and the organic layer was washed with brine, dried over anhydrous sodium sulfate, filtered and stripped. The crude was purified by semi-preparative HPLC (C18 column, acetonitrile/deionized water) to obtain compound 161 (4.2 mg y=12%) as a white solid. MS (m/z): 630.9 (M+1)⁺. UPLC=2.5 min (7 min method, 30-98%) Agilent.

### Example 31. Synthesis of Compound 162

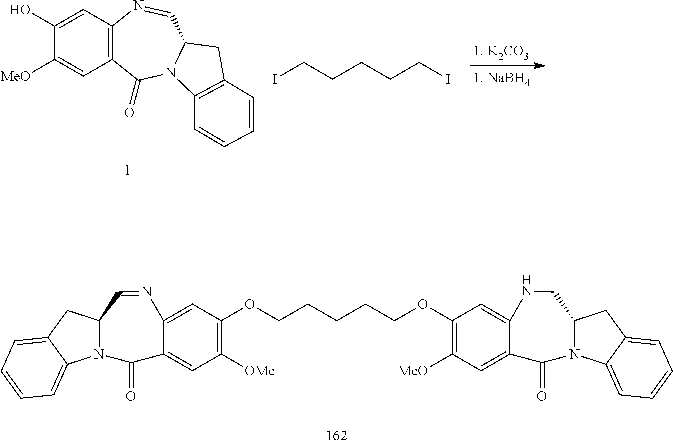

Compound 162 was prepared in a similar fashion to the two step procedure described in Example 30 to obtain compound 62 (0.055 g, 0.083 mmol, 34.3% yield). MS (m/z): 659.1 (M+1)⁺.

### Example 32. Preparation of Anti-FRα Conjugate of Compound 18 (Anti-FRα-18)

A reaction containing 2.0 mg/mL anti-FRα antibody and 3.4 molar equivalents compound 18 (pretreated with 5-fold excess of sodium bisulfite in 5% aqueous dimethylacetamide for 6 hours at 25° C.) in 50 mM HEPES (4-(2-hydroxyethyl)-1-piperazine ethanesulfonic acid) pH 8.5 buffer and 10% v/v dimethylacetamide (N,N-Dimethylacetamide) cosolvent was incubated for 8 hours at 25° C. Post-reaction, the conjugate was purified and buffer exchanged into 10 mM histidine, 250 mM glycine, 1.0% w/v sucrose, 0.01% Tween-20, 50 μM sodium bisulfite pH 5.5 formulation buffer using NAP desalting columns (Illustra Sephadex G-25 DNA Grade, GE Healthcare). Dialysis was performed in the same buffer for 4 hours at room temperature and then overnight at 4° C. utilizing Slide-a-Lyzer dialysis cassettes (ThermoScientific 30,000 MWCO).

The purified conjugate was found to have a final protein concentration of 1.2 mg/ml and an average of 2.6 compound 18 molecules linked per antibody (by UV-Vis using molar extinction coefficients ε_{330 nm}=11, 971 cm⁻¹M⁻¹ and ε₂₈₀ nm=30, 188 cm⁻¹M⁻¹ for compound 18, and ε₂₈₀ nm=201,400 cm⁻¹ M⁻¹ for anti-FRα antibody); 97.5% monomer (by size exclusion chromatography); and <1.1% unconjugated compound 18 (by dual column, reverse-phase HPLC analysis).

### Example 33. Preparation of Anti-EGFR Conjugate of Compound 79 (Anti-EGFR-79)

A reaction containing 2.0 mg/mL anti-EGFR antibody and 4.5 molar equivalents compound 79 (pretreated with 5-fold excess of sodium bisulfite in 5% aqueous dimethylacetamide for 6 hours at 25° C.) in 50 mM HEPES (4-(2-hydroxyethyl)-1-piperazine ethanesulfonic acid) pH 8.5 buffer and 10% v/v dimethylacetamide (N,N-Dimethylacetamide) cosolvent was incubated for 8 hours at 25° C. Post-reaction, the conjugate was purified and buffer exchanged into 10 mM histidine, 250 mM glycine, 1.0% w/v sucrose, 0.01% Tween-20, 50 μM sodium bisulfite pH 5.5 formulation buffer using NAP desalting columns (Illustra Sephadex G-25 DNA Grade, GE Healthcare). Dialysis was performed in the same buffer for 4 hours at room temperature and then overnight at 4° C. utilizing Slide-a-Lyzer dialysis cassettes (ThermoScientific 30,000 MWCO).

The purified conjugate was found to have a final protein concentration of 1.2 mg/ml and an average of 3.4 compound 79 molecules linked per antibody (by UV-Vis using molar extinction coefficients ε_{330 nm}=11, 971 cm⁻¹M⁻¹ and ε₂₈₀ nm=30, 188 cm⁻¹M⁻¹ for compound 79, and ε₂₈₀ nm=201,400 cm⁻¹M⁻¹ for anti-EGFR antibody); 96.3% monomer (by size exclusion chromatography); and <1.0% unconjugated compound 79 (by dual column, reverse-phase HPLC analysis).

### Example 34. Preparation of Anti-FRα Conjugate of Compound 46 (Anti-FRα-46)

A reaction containing 2.0 mg/mL anti-FRα antibody and 5 molar equivalents compound 46 (pretreated with 5-fold excess of sodium bisulfite in 5% aqueous dimethylacetamide for 6 hours at 25° C.) in 50 mM HEPES (4-(2-hydroxyethyl)-1-piperazine ethanesulfonic acid) pH 8.5 buffer and 10% v/v dimethylacetamide (N,N-Dimethylacetamide) cosolvent was incubated for 8 hours at 25° C. Post-reaction, the conjugate was purified and buffer exchanged into 10 mM histidine, 250 mM glycine, 1.0% w/v sucrose, 0.01% Tween-20, 50 μM sodium bisulfite pH 5.5 formulation buffer using NAP desalting columns (Illustra Sephadex G-25 DNA Grade, GE Healthcare). Dialysis was performed in the same buffer for 4 hours at room temperature and then overnight at 4° C. utilizing Slide-a-Lyzer dialysis cassettes (ThermoScientific 30,000 MWCO).

The purified conjugate was found to have a final protein concentration of 1.2 mg/ml and an average of 3.3 compound 46 molecules linked per antibody (by UV-Vis using molar extinction coefficients ε_{330 nm}=15,280 cm⁻¹M⁻¹ and ε₂₈₀ nm=30, 115 cm⁻¹M⁻¹ for compound 46, and ε₂₈₀ nm=201,400 cm⁻¹ M⁻¹ for anti-FRα antibody); 95.1% monomer (by size exclusion chromatography); and <0.8% unconjugated compound 46 (by dual column, reverse-phase HPLC analysis).

### Example 35. Preparation of Anti-FRα Conjugate of Compound 55 (Anti-FRα-55)

A reaction containing 2.0 mg/mL anti-FRα antibody and 5 molar equivalents compound 55 (pretreated with 5-fold excess of sodium bisulfite in 5% aqueous dimethylacetamide for 6 hours at 25° C.) in 50 mM HEPES (4-(2-hydroxyethyl)-1-piperazine ethanesulfonic acid) pH 8.5 buffer and 10% v/v dimethylacetamide (N,N-Dimethylacetamide) cosolvent was incubated for 8 hours at 25° C. Post-reaction, the conjugate was purified and buffer exchanged into 10 mM histidine, 250 mM glycine, 1.0% w/v sucrose, 0.01% Tween-20, 50 μM sodium bisulfite pH 5.5 formulation buffer using NAP desalting columns (Illustra Sephadex G-25 DNA Grade, GE Healthcare). Dialysis was performed in the same buffer for 4 hours at room temperature and then overnight at 4° C. utilizing Slide-a-Lyzer dialysis cassettes (ThermoScientific 30,000 MWCO).

The purified conjugate was found to have a final protein concentration of 2.5 mg/ml and an average of 2.4 compound 55 molecules linked per antibody (by UV-Vis using molar extinction coefficients ε_{330 nm}=15,280 cm⁻¹M⁻¹ and ε_{280 nm}=30, 115 cm⁻¹M⁻¹ for compound 55, and c_{280 nm}=201,400 cm⁻¹ M⁻¹ for anti-FRα antibody); 95.1% monomer (by size exclusion chromatography); and <0.8% unconjugated compound 55 (by dual column, reverse-phase HPLC analysis).

### Example 36. General Preparation of Anti-FRα-C442 or Anti-EGFR-C442 Conjugates of Compound 19 (Anti-FRα-19, Anti-EGFR-19) and Compound 47 (Anti-FRα-47, Anti-EGFR-47)

Antibody bearing two unpaired cysteine residues was prepared according to standard procedures.

To a solution of this intermediate in 15 mM potassium phosphate, 5 mM N,N,N′,N′-ethylenediaminetetraacetic acid (EDTA) pH 6.0 was added propylene glycol and 5-10 eq maleimide (compound 19 or compound 47) as a stock solution in N,N-dimethylacetamide (dimethylacetamide) to give a reaction mixture with a final solvent composition of ˜48% propylene glycol and ˜2% dimethylacetamide in 15 mM potassium phosphate, 5 mM EDTA pH 6.0. The reaction was allowed to proceed overnight at 25° C. The conjugate was purified into 20 mM succinate, 8.5% sucrose, 0.01% Tween-20, 50 μM sodium bisulfite pH 4.2 using Sephadex G-25 desalting columns. Purification was repeated as needed to remove residual unconjugated drug.

The purified conjugate was found to have a final protein concentration of ˜2.5 mg/ml and generally an average of ˜1.8 IGN molecules linked per antibody (by UV-Vis using molar extinction coefficients as above; ˜94% monomer (by size exclusion chromatography); and <1% unconjugated IGN (by dual column, reverse-phase HPLC analysis).

### Example 37. Cytotoxicity Assay

The following cell lines were used for the studies: KB (cervical carcinoma, ATCC), NCI-H2110 (Non Small Cell Lung Carcinoma, ATCC), Namalwa (Burkitt's lymphoma, ATCC), and T47D (breast epithelial cancer, ATCC). The cells were maintained and plated for the cytotox experiments in media recommended by the manufacturers. Cells were plated in the 96-well flat bottom plates at a seeding density of 1,000 cells per well (KB, Namalwa) or 2,000 cell per well (NCI H2110, T47D). Conjugates or free drug compounds were diluted in RPMI-1640 (Life Technologies) supplemented with heat-inactivated 10% FBS (Life Technologies) and 0.1 mg/ml gentamycin (Life Technologies), and added to the plated cells. To determine specificity of cytotoxic activity of the conjugates an excess of unconjugated antibody was added to a separate set of diluted conjugates (+block samples, IC₅₀ table). The plates were incubated at 37° C., 5% CO₂ for either 4 days (T47D cells) or 5 days (KB, NCI H2110 cells). Alamar blue assay (Invitrogen) was used to determine viability of T47D cells, and WST-8 assay (Donjindo Molecular Technologies, Inc.) was applied for viability of KB, NCI H2110, Namalwa cells. The assays were performed in accordance with the manufacturer's protocols. Killing curves and IC₅₀ were generated using a sigmoidal dose-response nonlinear regression curve fit (GraphPad Software Inc.). As shown in Tables 1-3, the cytotoxic compounds and conjugates of the present invention are highly potent against various cancer cells in in vitro cytotoxicity assays.

TABLE 1
IC₅₀ values for free cytotoxic compounds (Molar,
M) determined by in vitro cytotoxicity assays.

| IGN Catabolite | KB | Namalwa |
|---|---|---|
| 150 | 8.00E−12 | 1.00E−12 |
| 162 | 2.00E−11 | 3.00E−12 |
| 157 | 3.00E−12 | 1.00E−12 |
| 155 | 2.00E−11 | 2.00E−12 |
| 161 | 2.50E−10 | 3.00E−11 |
| 159 | 3.00E−12 | 1.00E−12 |
| 123 | 3.00E−12 | 1.00E−12 |
| 108 | 5.00E−11 | 1.00E−11 |

TABLE 2
IC₅₀ values (Molar, M) for anti-FRα-Conjugates
determined by in vitro cytotoxicity assays.

| Peptide | Com- | KB | KB | T47D | T47D |
|---|---|---|---|---|---|
| Linker | pound # | −block | +block | −block | +block |
| Ala-Ala | 95 | 2.00E−11 | 2.00E−9 | 3.00E−10 | 4.00E−9 |
| Ala-Val | 18 | 3.00E−11 | 3.00E−9 | 1.00E−10 | 1.00E−8 |
| Ala-Val | 79 | 1.00E−10 | >3.00E−8 | | |
| Ala-Val | 83 | 7.00E−12 | 2.00E−9 | 7.00E−11 | 1.00E−8 |
| Gln-Leu | 126 | 1.00E−11 | >3.50E−9 | 5.00E−11 | 1.00E−8 |
| Ala-Val | 23 | 2.00E−12 | 4.00E−10 | 6.00E−12 | 2.00E−9 |
| Gln-Leu | 55 | 1.00E−11 | 2.00E−9 | 2.00E−10 | 7.00E−9 |
| Ala-Val | 26 | 2.00E−11 | >3.50E−9 | 3.00E−11 | 9.00E−9 |
| Ala-Val | 46 | 2.00E−11 | 3.50E−9 | 3.00E−10 | 2.00E−8 |
| Ala-Val | 65 | 1.00E−12 | 7.00e−10 | 8.00E−12 | 7.00e−9 |
| — | 110 | 2.00E−11 | 6.00E−11 | 2.00E−10 | 8.00E−10 |
| — | 121 | 1.00E−11 | 1.00E−10 | 5.00E−10 | 2.00E−9 |
| — | 117 | 1.00E−10 | 9.00E−10 | 3.00E−9 | 3.00E−9 |
| — | 148 | 3.00E−11 | 4.00E−10 | 5.00E−9 | 5.00E−9 |
| Ala-Val | 99 | 2.00E−12 | 6.00E−10 | 1.00E−11 | 3.00E−9 |
| Ala-Val | 106 | 7.00E−11 | >3.50E−9 | 2.00E−10 | 3.00E−8 |

TABLE 3
IC₅₀ values (Molar, M) for anti-EGFR-Conjugates determined by in vitro cytotoxicity assays.

| Peptide | | HSC2 − | HSC2 + | KB − | KB + | H2110 − | H2110 + |
|---|---|---|---|---|---|---|---|
| Linker | Compound # | block | block | block | block | block | block |
| Ala-Val | 18 | 6.00E−12 | 1.00E−9 | 4.00E−11 | 3.50E−9 | 4.00E−10 | 8.00E−9 |
| Ala-Val | 79 | 3.00E−10 | >3.50E−9 | 3.00E−9 | >3.50E−9 | >1.00E−8 | >3.50E−8 |
| Gln-Leu | 55 | 4.00E−12 | 9.00E−10 | 3.00E−11 | 2.00E−9 | 1.00E−10 | 6.00E−9 |
| Ala-Val | 46 | 6.00E−12 | 1.00E−9 | 4.00E−11 | 2.00E−9 | 1.00E−10 | 1.00E−8 |
| Ala-Val | 65 | 1.50E−12 | 1.20E−9 | 4.80E−12 | 1.50E−9 | | |
| Ala-Val | 134 | 9.00E−11 | 8.00E−9 | 6.00E−10 | 1.00E−8 | | |
| Ser-Val | 147 | 5.00E−11 | 5.00E−9 | 2.50E−10 | 7.00E−9 | | |

### Example 38. Anti-Tumor Activity (Median Tumor Volume, Mm³) of Anti-FRα-55 in SCID Mice Bearing OV90 Xenografts

Female CB.17 SCID mice, 6 weeks old, were received from Charles River Laboratories. Mice were inoculated with 1×10⁷ OV-90 tumor cells suspended in 0.1 ml 50% matrigel/serum free medium by subcutaneous injection in the right flank. When tumor volumes reached approximately 100 mm³ (day 7 post inoculation), animals were randomized based on tumor volume into 4 groups of 6 mice each. Mice received a single IV administration of vehicle control (0.15 ml/mouse) or anti-FRα-55 at 0.7, 1.4 or 2.7 mg/kg on day 0 (day 7 post inoculation).

Tumor size was measured twice to three times weekly in three dimensions using a caliper. The tumor volume was expressed in mm³ using the formula V=Length×Width×Height×½. A mouse was considered to have a partial regression (PR) when tumor volume was reduced by 50% or greater, complete tumor regression (CR) when no palpable tumor could be detected. Tumor volume was determined by StudyLog software.

Tumor growth inhibition (T/C Value) was determined using the following formula:

T/C(%)=Median tumor volume of the treated/Median tumor volume of the control×100.

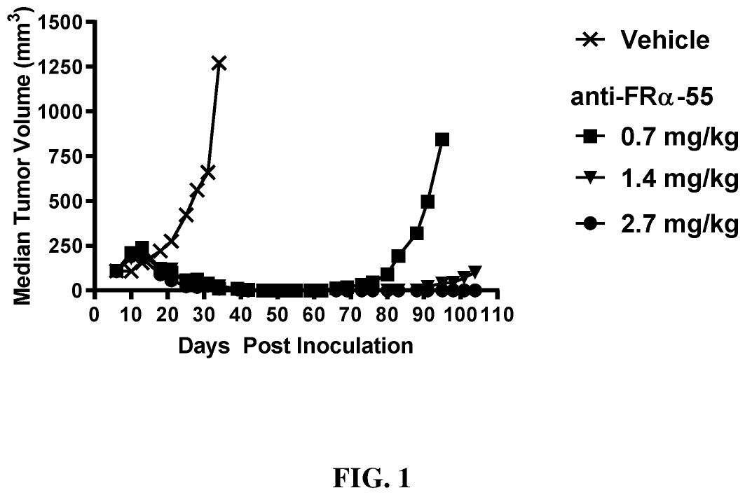

Tumor volume was determined simultaneously for treated (T) and the vehicle control (C) groups when tumor volume of the vehicle control reached predetermined size of 1000 mm³. The daily median tumor volume of each treated group was determined, including tumor-free mice (0 mm³). According to NCI standards, a T/C≤42% is the minimum level of anti-tumor activity. A T/C<10% is considered a high anti-tumor activity level.
As shown in FIG. 1 the anti-FRα-55 conjugate is highly active at all doses tested.

### Example 39. Anti-Tumor Activity (Median Tumor Volume, Mm³) of Anti-FRα-18 in SCID Mice Bearing NCI-H2110 Xenografts

Female CB.17 SCID mice, 6 weeks old, were received from Charles River Laboratories. Mice were inoculated with 1×10⁷ NCI-H2110 tumor cells suspended in 0.1 ml 50% matrigel/serum free medium by subcutaneous injection in the right flank. When tumor volumes reached approximately 100 mm³ (day 6 post inoculation), animals were randomized based on tumor volume into 3 groups of 6 mice each. Mice received a single IV administration of vehicle control (0.15 ml/mouse) or anti-FRα-18 at 1.1 or 2.2 mg/kg on day 0 (day 6 post inoculation).

Tumor size was measured twice to three times weekly in three dimensions using a caliper. The tumor volume was expressed in mm³ using the formula V=Length×Width×Height×½. A mouse was considered to have a partial regression (PR) when tumor volume was reduced by 50% or greater, complete tumor regression (CR) when no palpable tumor could be detected. Tumor volume was determined by StudyLog software.

Tumor growth inhibition (T/C Value) was determined using the following formula:

T/C(%)=Median tumor volume of the treated/Median tumor volume of the control×100.

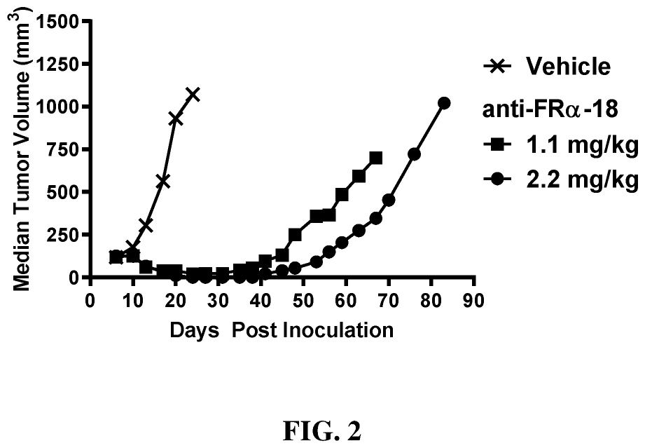

Tumor volume was determined simultaneously for treated (T) and the vehicle control (C) groups when tumor volume of the vehicle control reached predetermined size of 1000 mm³. The daily median tumor volume of each treated group was determined, including tumor-free mice (0 mm³). According to NCI standards, a T/C≤42% is the minimum level of anti-tumor activity. A T/C<10% is considered a high anti-tumor activity level.
As shown in FIG. 2, the anti-FRα-18 conjugate is highly active at all doses tested.

### Example 40. Anti-Tumor Activity (Median Tumor Volume, Mm³) of Anti-EGFR-79 in SCID Mice Bearing FaDu Xenografts

Female CB.17 SCID mice, 6 weeks old, were received from Charles River Laboratories. Mice were inoculated with 1×10⁷ FaDu tumor cells suspended in 0.1 ml 50% matrigel/serum free medium by subcutaneous injection in the right flank. When tumor volumes reached approximately 100 mm³ (day 6 post inoculation), animals were randomized based on tumor volume into 4 groups of 6 mice each. Mice received a single IV administration of vehicle control (0.15 ml/mouse) or anti-EGFR-79 at 3.7, 7.4 or 14.9 mg/kg on day 0 (day 6 post inoculation).

Tumor size was measured twice to three times weekly in three dimensions using a caliper. The tumor volume was expressed in mm³ using the formula V=Length×Width×Height×½. A mouse was considered to have a partial regression (PR) when tumor volume was reduced by 50% or greater, complete tumor regression (CR) when no palpable tumor could be detected. Tumor volume was determined by StudyLog software.

Tumor growth inhibition (T/C Value) was determined using the following formula:

T/C(%)=Median tumor volume of the treated/Median tumor volume of the control×100.

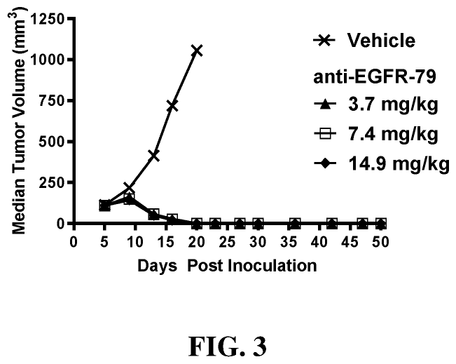

Tumor volume was determined simultaneously for treated (T) and the vehicle control (C) groups when tumor volume of the vehicle control reached predetermined size of 1000 mm³. The daily median tumor volume of each treated group was determined, including tumor-free mice (0 mm³). According to NCI standards, a T/C≤42% is the minimum level of anti-tumor activity. A T/C<10% is considered a high anti-tumor activity level.
As shown in FIG. 3, the anti-EGFR-79 conjugate is highly active at all doses tested.

### Example 41. Anti-Tumor Activity (Median Tumor Volume, Mm³) of Anti-FRα-46 in SCID Mice Bearing Ishikawa Xenografts

Female CB.17 SCID mice, 6 weeks old, were received from Charles River Laboratories. Mice were inoculated with 1×10⁷ Ishikawa tumor cells suspended in 0.1 ml 50% matrigel/serum free medium by subcutaneous injection in the right flank. When tumor volumes reached approximately 100 mm³ (day 23 post inoculation), animals were randomized based on tumor volume into 4 groups of 6 mice each. Mice received a single IV administration of vehicle control (0.15 ml/mouse) or anti-FRα-46 at 0.5, 0.9, and 1.9 mg/kg on day 0 (day 23 post inoculation).

Tumor size was measured twice to three times weekly in three dimensions using a caliper. The tumor volume was expressed in mm using the formula V=Length×Width×Height×½. A mouse was considered to have a partial regression (PR) when tumor volume was reduced by 50% or greater, complete tumor regression (CR) when no palpable tumor could be detected. Tumor volume was determined by StudyLog software.
Tumor growth inhibition (T/C Value) was determined using the following formula:

T/C(%)=Median tumor volume of the treated/Median tumor volume of the control×100.

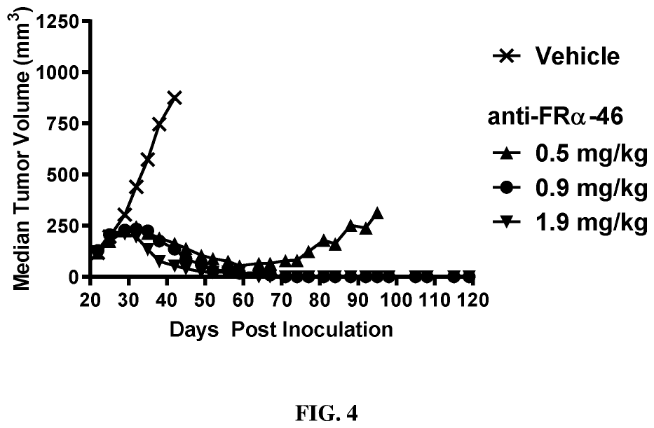

Tumor volume was determined simultaneously for treated (T) and the vehicle control (C) groups when tumor volume of the vehicle control reached predetermined size of 1000 mm³. The daily median tumor volume of each treated group was determined, including tumor-free mice (0 mm³). According to NCI standards, a T/C≤42% is the minimum level of anti-tumor activity. A T/C<10% is considered a high anti-tumor activity level.
As shown in FIG. 4, the anti-FRα-46 conjugate is active at all doses tested.

### Example 42. Anti-Tumor Activity (Median Tumor Volume, Mm³) of Anti-FRα-18 in SCID Mice Bearing KB Xenografts

Female CB.17 SCID mice, 6 weeks old, were received from Charles River Laboratories. Mice were inoculated with 1×10⁷ Ishikawa tumor cells suspended in 0.1 ml 50% matrigel/serum free medium by subcutaneous injection in the right flank. When tumor volumes reached approximately 100 mm³ (day 6 post inoculation), animals were randomized based on tumor volume into 3 groups of 6 mice each. Mice received a single IV administration of vehicle control (0.15 ml/mouse) or anti-FRα-18 at 1.3 and 2.7 mg/kg on day 0 (day 6 post inoculation).

Tumor size was measured twice to three times weekly in three dimensions using a caliper. The tumor volume was expressed in mm using the formula V=Length×Width×Height×½. A mouse was considered to have a partial regression (PR) when tumor volume was reduced by 50% or greater, complete tumor regression (CR) when no palpable tumor could be detected. Tumor volume was determined by StudyLog software.
Tumor growth inhibition (T/C Value) was determined using the following formula:

T/C(%)=Median tumor volume of the treated/Median tumor volume of the control×100.

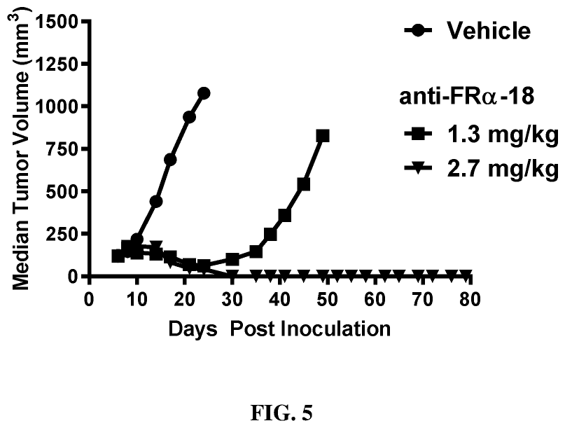

Tumor volume was determined simultaneously for treated (T) and the vehicle control (C) groups when tumor volume of the vehicle control reached predetermined size of 1000 mm³. The daily median tumor volume of each treated group was determined, including tumor-free mice (0 mm³). According to NCI standards, a T/C≤42% is the minimum level of anti-tumor activity. A T/C<10% is considered a high anti-tumor activity level.
As shown in FIG. 5, the anti-FRα-18 conjugate is highly active at all doses tested.

### Example 43. Anti-Tumor Activity (Median Tumor Volume, Mm³) of Anti-FRα-46 in SCID Mice Bearing KB Xenografts

Female CB.17 SCID mice, 6 weeks old, were received from Charles River Laboratories. Mice were inoculated with 1×10⁷ KB tumor cells suspended in 0.1 ml 50% matrigel/serum free medium by subcutaneous injection in the right flank. When tumor volumes reached approximately 100 mm³ (day 6 post inoculation), animals were randomized based on tumor volume into 4 groups of 6 mice each. Mice received a single IV administration of vehicle control (0.15 ml/mouse) or anti-FRα-46 at 0.5, 0.9 and 1.9 mg/kg on day 0 (day 6 post inoculation).

Tumor size was measured twice to three times weekly in three dimensions using a caliper. The tumor volume was expressed in mm using the formula V=Length×Width×Height×½. A mouse was considered to have a partial regression (PR) when tumor volume was reduced by 50% or greater, complete tumor regression (CR) when no palpable tumor could be detected. Tumor volume was determined by StudyLog software.
Tumor growth inhibition (T/C Value) was determined using the following formula:

T/C(%)=Median tumor volume of the treated/Median tumor volume of the control×100.

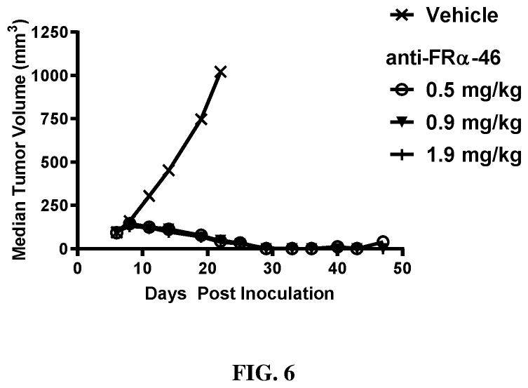

Tumor volume was determined simultaneously for treated (T) and the vehicle control (C) groups when tumor volume of the vehicle control reached predetermined size of 1000 mm³. The daily median tumor volume of each treated group was determined, including tumor-free mice (0 mm³). According to NCI standards, a T/C≤42% is the minimum level of anti-tumor activity. A T/C<10% is considered a high anti-tumor activity level.
As shown in FIG. 6, the anti-FRα-46 conjugate is highly active at all doses tested.

### Example 44. Anti-Tumor Activity (Median Tumor Volume, Mm³) of huCD19-55 in SCID Mice Bearing OCI-Ly18 Xenografts

Female CB.17 SCID mice, 6 weeks old, were received from Charles River Laboratories. Mice were inoculated with 1×10⁷ OCI-Ly18 tumor cells suspended in 0.1 ml 50% matrigel/serum free medium by subcutaneous injection in the right flank. When tumor volumes reached approximately 100 mm³ (day 14 post inoculation), animals were randomized based on tumor volume into 4 groups of 6 mice each. Mice received a single IV administration of vehicle control (0.15 ml/mouse) or huCD19-55 at 1.1 and 2.1 mg/kg on day 0 (day 14 post inoculation).

Tumor size was measured twice to three times weekly in three dimensions using a caliper. The tumor volume was expressed in mm using the formula V=Length×Width×Height×½. A mouse was considered to have a partial regression (PR) when tumor volume was reduced by 50% or greater, complete tumor regression (CR) when no palpable tumor could be detected. Tumor volume was determined by StudyLog software.
Tumor growth inhibition (T/C Value) was determined using the following formula:

T/C(%)=Median tumor volume of the treated/Median tumor volume of the control×100.

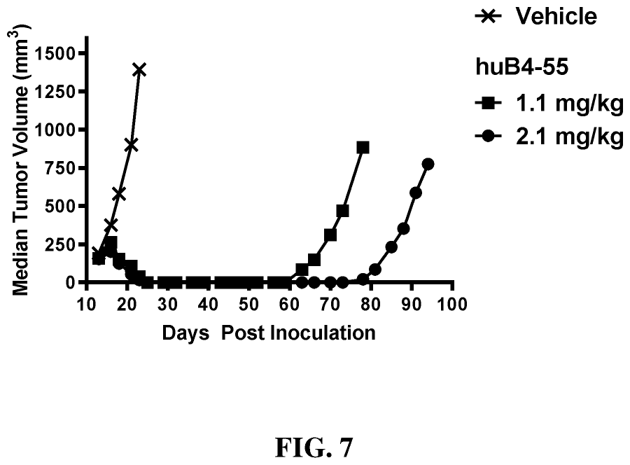

Tumor volume was determined simultaneously for treated (T) and the vehicle control (C) groups when tumor volume of the vehicle control reached predetermined size of 1000 mm³. The daily median tumor volume of each treated group was determined, including tumor-free mice (0 mm³). According to NCI standards, a T/C≤42% is the minimum level of anti-tumor activity. A T/C<10% is considered a high anti-tumor activity level.
As shown in FIG. 7, the huCD19-55 conjugate is highly active at all doses tested.

### Example 45. Bystander Activity of Selected Anti-EGFR-IGN Conjugates

Cells were plated in the 96-well U-bottom, low cluster plates (Costar) at a seeding density of 1000 MDA-MB-468 cells per well or 500 Namalwa cells per well in RPMI-1640 (Thermo Fisher) supplemented with heat-inactived 10% FBS (Thermo Fisher) and 0.1 mg/ml gentamycin (Thermo Fisher). A mixed culture of antigen positive MDA-MDA-468 cells and antigen negative Namalwa cells (or each cell cultured separately) was exposed to serial diluted conjugates in same media and added to the plated cells. Cells were incubated for 5 days, and the inhibition of total cell viability was determined by Cell Titer Glo (Promega) according to the manufacturer's protocol. Results, as shown in Table 4, indicate that all conjugates demonstrate bystander activity on antigen negative cells

TABLE 4
IC₅₀ values (Molar, M) for anti-EGFR-Conjugates determined
by in vitro total cell viability assays.

| Compound | Controls, IC₅₀, M | | Bystander |
|---|---|---|---|
| # | Namalwa | MDA-MB-468 | (MDA-MB-468 + Namalwa) |
| 18 | 2.00E−9 | 2.30E−11 | 3.00E−11 |
| 79 | 6.00E−9 | 1.70E−10 | 2.70E−10 |
| 46 | 3.00E−9 | 1.00E−11 | 1.00E−11 |

### Example 46. Binding of Catabolites of the Compounds of the Invention to DNA

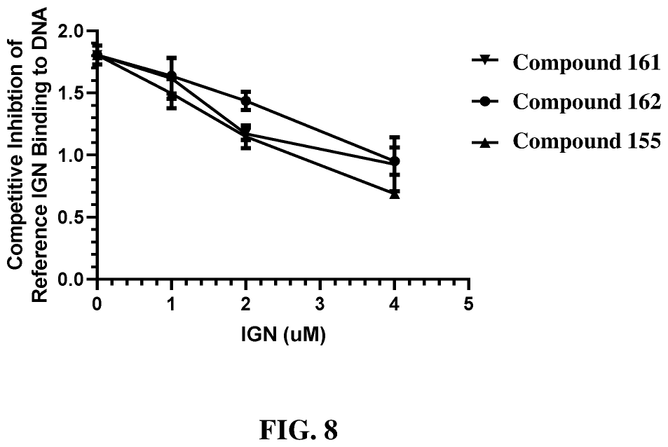

The binding of catabolites of the compounds of the invention to a digoxigenin-labeled hairpin oligonucleotide was measured using a competition ELISA, in which the catabolite at several concentrations was pre-mixed with a biotinylated reference molecule followed by incubation with digoxigenin-labeled hairpin oligonucleotide. The binding of the biotinylated reference compound to the digoxigenin-labeled hairpin oligonucleotide, in the presence of competing catabolites of the invention, was assessed by ELISA. The ELISA method used coated streptavidin for capture and anti-digoxigenin antibody-horseradish peroxidase (HRP) conjugate for detection. Results are shown in FIG. 8.

### Example 47. Comparative Data on Conjugation Efficiency

A comparator indolinobenzodiazepine compound, compound A, was synthesized using procedures similar to those described in WO2013/177481.

Compound A has a self-immolative linker at the N-10 position of the masked imine benzodiazepine monomer (i.e., cleavage of the self-immolative linker results in the formation of an imine bond at the N10-C11 position). In contrast, the compounds of the present invention, such as compound 18, has a self-immolative linker at the N-10 position of the reduced-imine benzodiazepine monomer. The compound of the present invention possesses an imine functionality in the benzodiazepine monomer that does not bear the self-immolative linker. The imine functionality can be modified by treating the compound with sodium bisulfite to form a sulfonated compound with increased solubility before the conjugation reaction with an antibody.

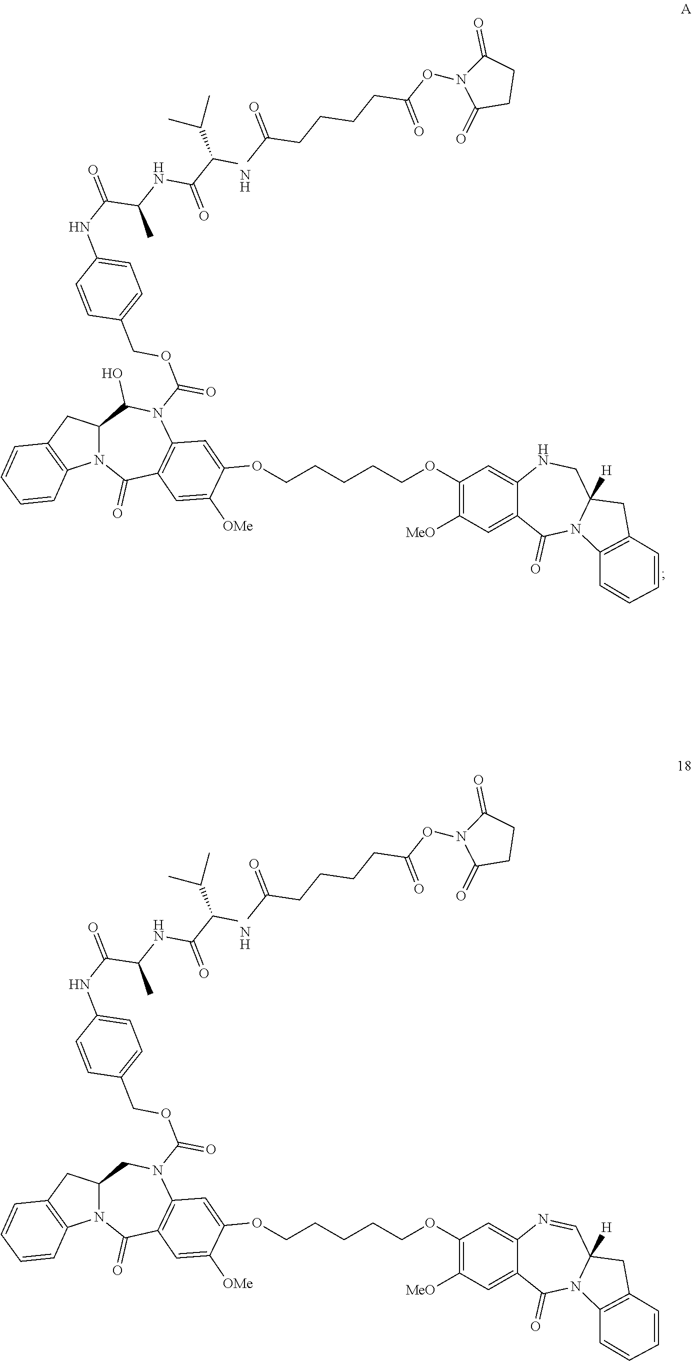

### Synthesis of Compound A

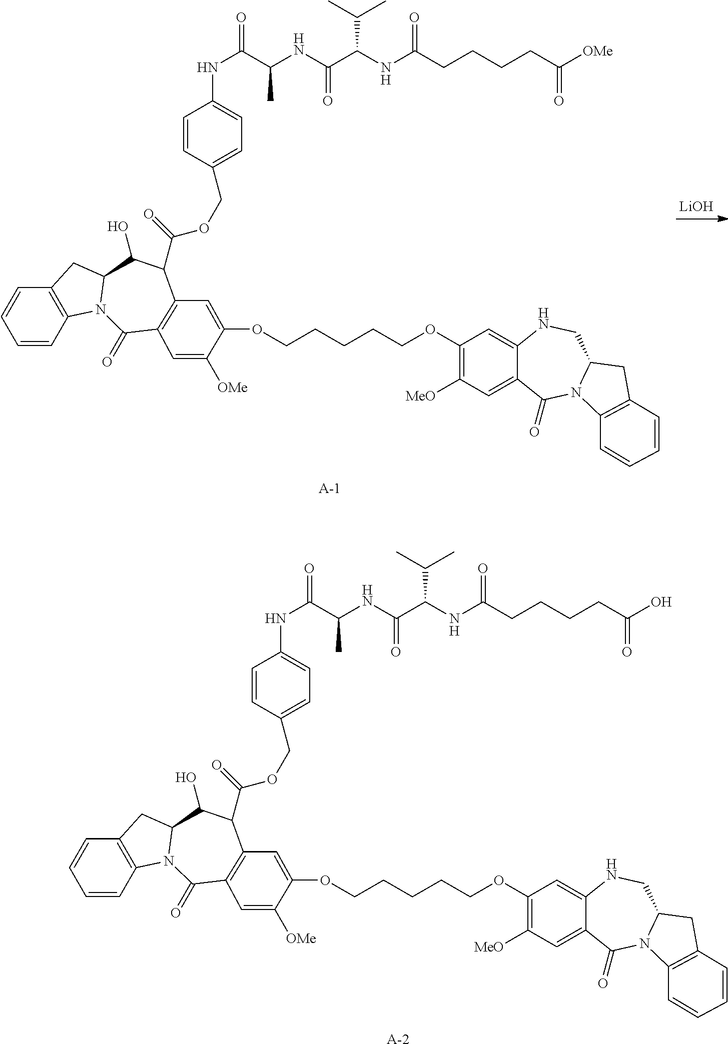

4-((S)-2-((S)-2-(6-methoxy-6-oxohexanamido)-3-methylbutanamido)propanamido)benzyl (12S,12aS)-12-hydroxy-8-methoxy-9-((5-(((S)-8-methoxy-6-oxo-11,12,12a,13-tetrahydro-6H-benzo[5,6][1,4]diazepino[1,2-a]indol-9-yl)oxy)pentyl)oxy)-6-oxo-12a,13-dihydro-6H-benzo[5,6][1,4]diazepino[1,2-a]indole-11(12H)-carboxylate (30 mg, 0.026 mmol) (compound A-1) was prepared using procedures similar to those described in WO2013/177481. Compound A-1 was dissolved in anhydrous tetrahydrofuran (988 μl) and DI water (329 μl). Lithium hydroxide (3.16 mg, 0.132 mmol) was added. The reaction mixture was monitored by LCMS. After stirring for 90 mins at room temp it was diluted with 30% MeOH/DCM and DI water, then acidified with 0.5 M HCl (˜1 mL) to pH-3. White precipitate formed. The solution was extracted 2 times with 30% MeOH/DCM. The organic layer was washed with water, dried with magnesium sulfate, filtered through celite and concentrated to obtain 20 mg crude product as an off white solid, compound A-2.

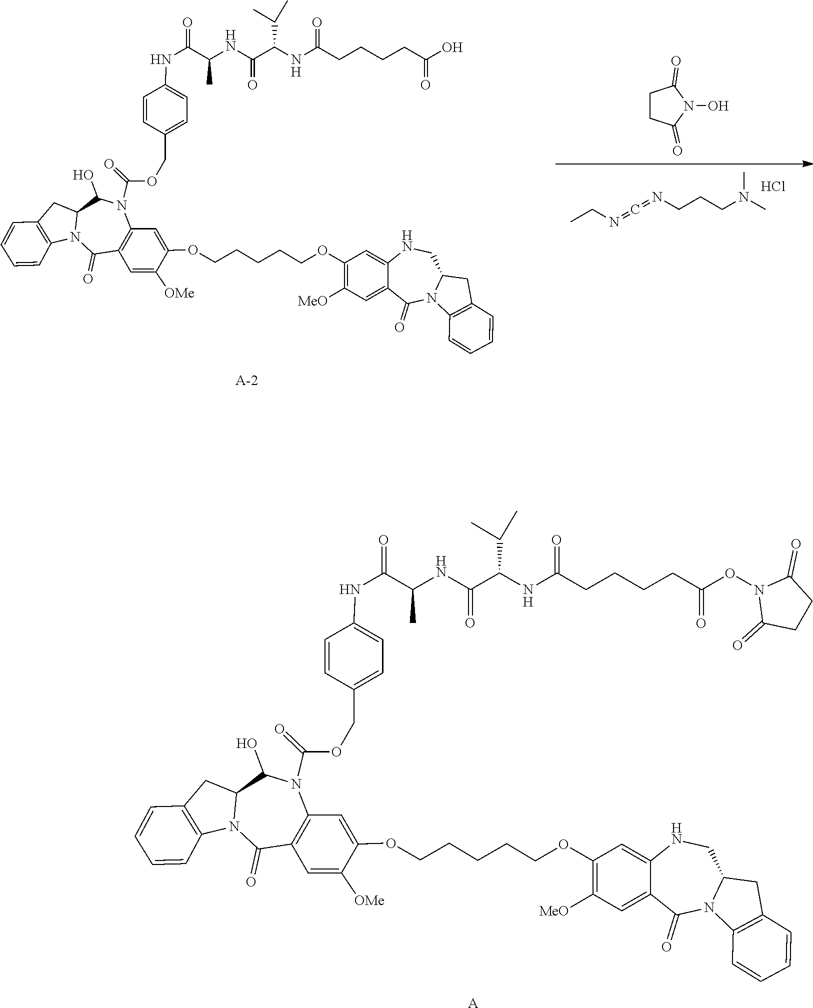

6-(((S)-1-(((S)-1-((4-((((12S,12aS)-12-hydroxy-8-methoxy-9-((5-(((S)-8-methoxy-6-oxo-11,12,12a,13-tetrahydro-6H-benzo[5,6][1,4]diazepino[1,2-a]indol-9-yl)oxy)pentyl)oxy)-6-oxo-11,12,12a,13-tetrahydro-6H-benzo[5,6][1,4]diazepino[1,2-a]indole-11-carbonyl)oxy)methyl)phenyl)amino)-1-oxopropan-2-yl)amino)-3-methyl-1-oxobutan-2-yl)amino)-6-oxohexanoic acid, compound A-2 (20 mg, 0.018 mmol) and N-hydroxy succinimide (6.14 mg, 0.053 mmol) were dissolved in anhydrous dichloromethane (356 μl). EDC•HCl (17.05 mg, 0.089 mmol) was added and the reaction mixture was stirred at room temperature for 90 mins. Upon completion of the reaction, the mixture was diluted with DCM and washed with water. The organic layer was dried with anhydrous magnesium sulfate, filtered and concentrated. The crude product was purified by RP-HPLC (C18 Kromasil, ACN/DI water, 50-65% over 30 mins). Fractions containing the product were combined, frozen and lyophilized to yield compound A.

### Preparation of Antibody Conjugate of Compound A

An antibody conjugate of compound A was prepared by reacting the antibody with excess compound A in a buffer solution containing 50 mM HEPES pH 8.5 and a cosolvent (15% or 30% v/v dimethylacetamide (DMA) or 40% propylene glycol (PG)) at room temperature for 24 hours or longer. Table 5 below shows the conjugation yields, monomer percentage and DAR values.

TABLE 5

| Conditions | Monomer | DAR | Yield |
|---|---|---|---|
| 50 mM HEPES, pH 8.5, 15% DMA, RT, | 61% | 1.3 | 45% |
| 24 h | | | |
| 50 mM HEPES, pH 8.5, 30% DMA, RT, | 87% | ~1.0 | NA |
| 24-72 h | | | |
| 50 mM HEPES, pH 8.5, 40% PG, RT, | 79% | ~1.1 | NA |
| 24-72 h | | | |

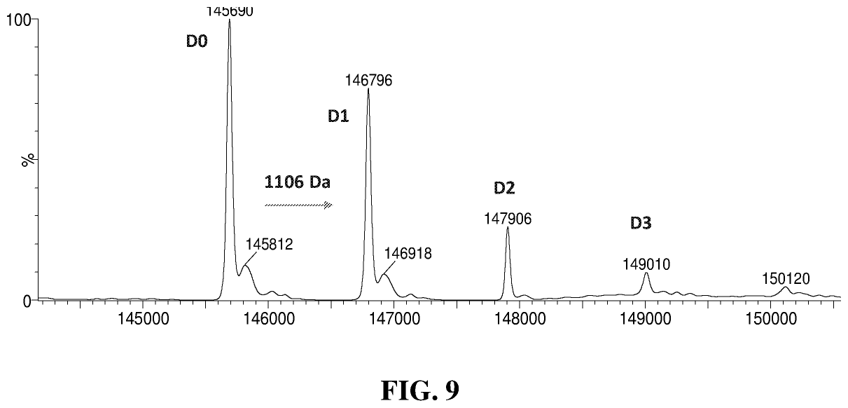

Partly due to the low solubility of compound A, the extent of incorporation of compound A in the conjugate was found to be much lower than the conjugates of the present invention, as indicated by significantly lower DAR values. By comparison, the DAR value of an antibody conjugate of compound 18 is ˜2.6 (see Example 32 above). In addition, FIG. 9 shows that there is a large amount of unconjugated antibody (DO) present in the resulting conjugate. It is also observed that the monomer percentage of the conjugate of compound A is much lower than the conjugate of the present invention. By comparison, the antibody conjugate of compound 18 has a monomer percentage of 97.5%; while the conjugate of compound A has a monomer percentage between 61% to 87%, depending on the reaction conditions used. Increasing the amount of co-solvent used in the conjugation reaction to solubilize compound A did not result in an increase in DAR value.

We claim:

1. A cytotoxic compound represented by the following formula: 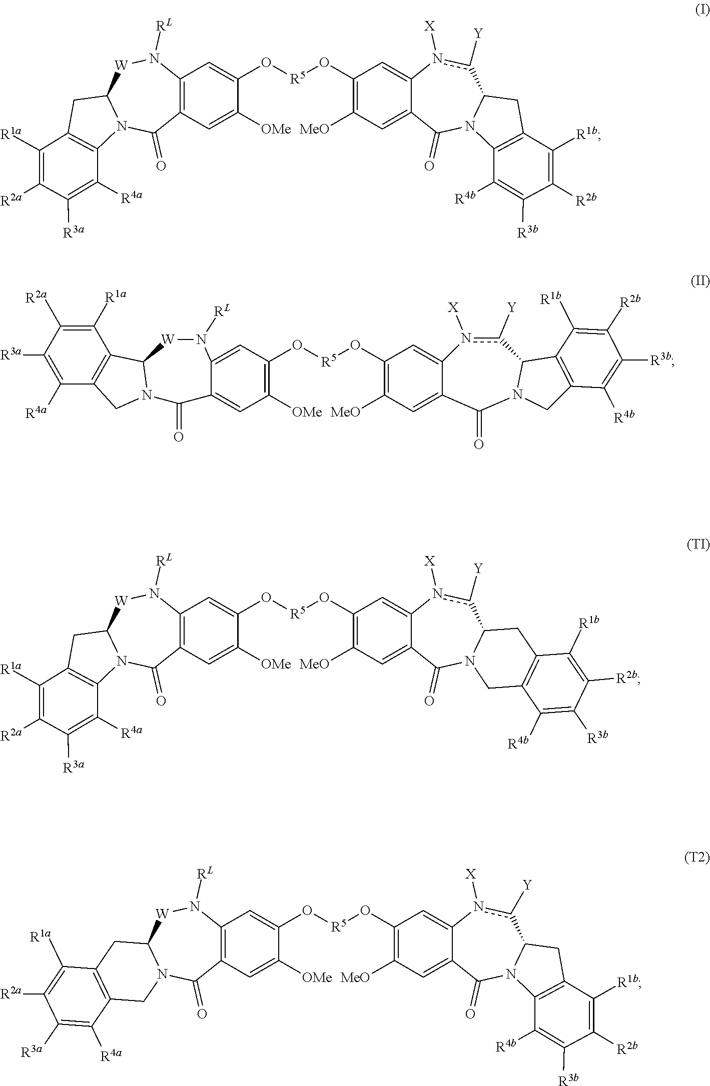 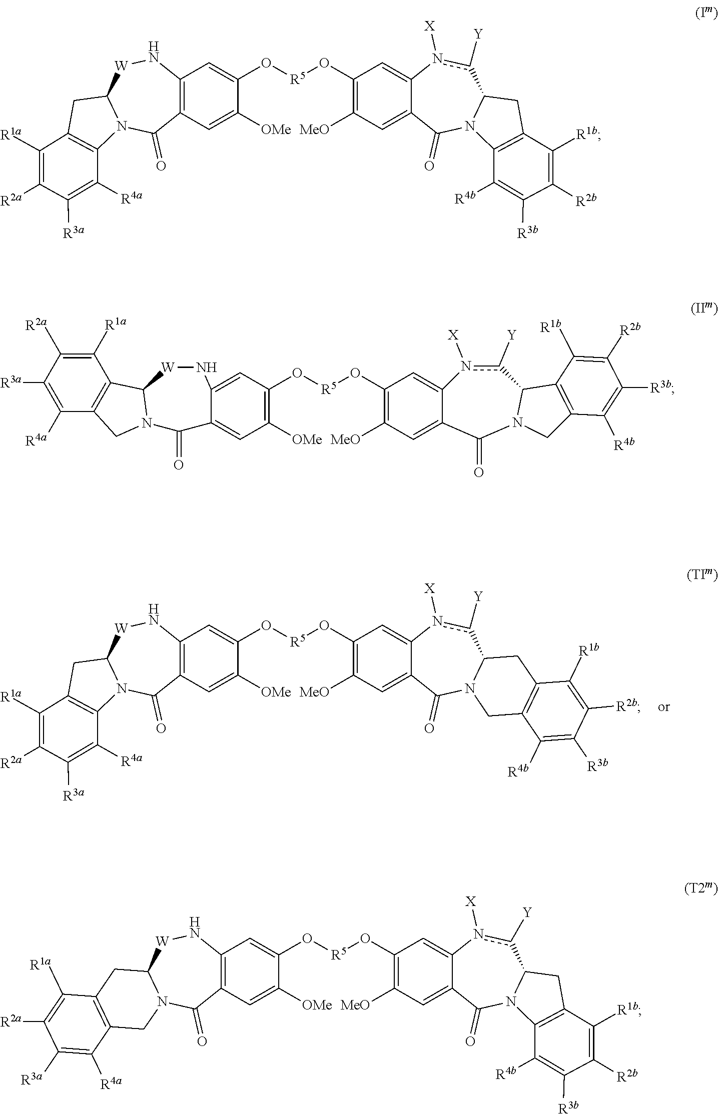
or a pharmaceutically acceptable salt thereof, wherein:
the double line [symbol] between N and C represents a single bond or a double bond, provided that when it is a double bond X is absent and Y is H, or a C₁₋₄alkyl, and when it is a single bond, X is H and Y is —OH or —SO₃H;
W is —C(═O)— or —C(Y′)—;
Y′ is H or C₁₋₄alkyl;
R^{1a}, R^{2a}, R^{3a}, R^{4a}, R^{1b}, R^{2b}, R^{3b} and R^{4b} are each independently selected from the group consisting of H, a C₁₋₁₀alkyl, —(OCH₂CH₂)ₙOR^{c}, halogen, —NH(C═NH)NH₂, —OR, —NR′R″, —NO₂, —NR′COR″, —SR, —SOR′, —SO₂R′, —SO₃H, —OSO₃H, —SO₂NR′R″, —CN, —N₃, —COR′, —OCOR′, and —OCONR′R″;
R^{c} is H or a C₁₋₄alkyl;
n is an integer from 1 to 24;
R, for each occurrence, is independently selected from the group consisting of H, —(CH₂CH₂O)ₙ—R^{c}, C₁₋₁₀alkyl, a C₃₋₈cycloalkyl, a 6- to 18-membered aryl, a 5- to 18-membered heteroaryl ring containing one or more heteroatoms independently selected from N, O and S, or a 3- to 18-membered heterocyclic ring containing 1 to 6 heteroatoms independently selected from O, S, N and P;
R′ and R″ are each independently selected from —H, —OH, —OR, —NHR, —NR₂, —COR, a C₁₋₁₀alkyl, a-(CH₂CH₂O)ₙ—R^{c}, and a 3- to 18-membered heterocyclic ring having 1 to 6 heteroatoms independently selected from O, S, N and P;
R⁵ is a C₃₋₁₂alkylene, which chain can be interrupted by one or more groups selected from —O—, —S—, —NH—, —NMe-, benzene ring, a 4 to 7-membered heteroaryl ring and a 4 to 7-membered heterocyclic ring, wherein the benzene, the 4 to 7-membered heteroaryl ring and the 4 to 7-membered heterocyclic ring are substituted with 1 to 4 R⁶;
R⁶ for each occurrence is independently selected from H, C₁₋₁₀alkyl, —(CH₂CH₂O)ₙ—R^{c}, halogen, —NH(C═NH)NH₂, —OR, —NR′R″, —NO₂, —NCO, —NR′COR″, —SR, —SOR′, —SO₂R′, —SO₃H, —OSO₃H, —SO₂NR′R″, —CN, —N₃, —COR′, —OCOR′, and —OCONR′R″; and
R^{L} is a self-immolative linker comprising a reactive group that can form a covalent bond with a cell-binding agent, provided that the compound of formula (I) is not: 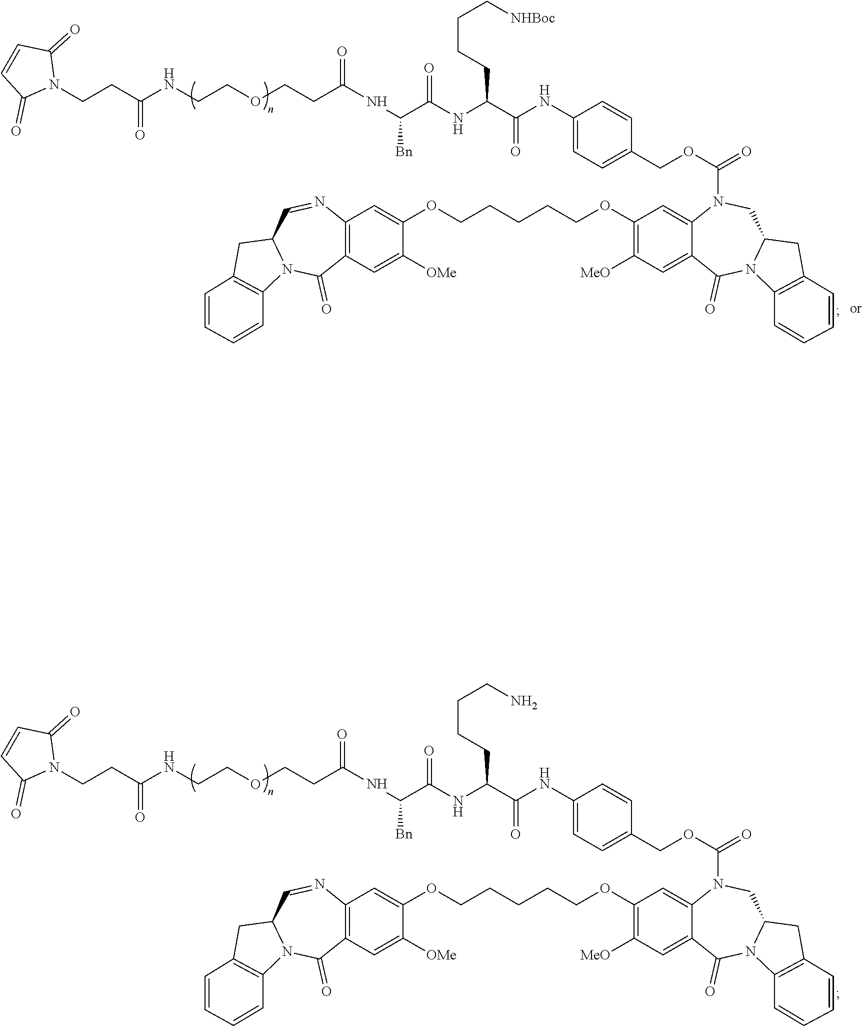
and provided the compound of formula (I^{m}) is not:

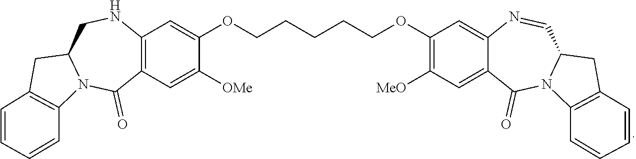 [SMILES: COc1cc2c(cc1OCCCCCOc1cc3c(cc1OC)C(=O)N1c4ccccc4C[C@H]1CN3)N=C[C@@H]1Cc3ccccc3N1C2=O]

2. The compound of claim 1, wherein the cytotoxic compound is represented by one of the formulae depicted in Table A, or a pharmaceutically acceptable salt thereof, wherein:
AA¹ and AA² are each independently an amino acid residues;
a1 is an integer from 1 to 19;
a2 is an integer from 1 to 5;
R^{a} is H or C₁₋₄alkyl;
q is 1, 2 or 3;
R^{s1} and R^{s2} are each independently H or C₁₋₄alkyl, or R^{s1} and R^{s2} taken together with the carbon atom to which they are attached form a 3 to 5-membered cycloalkyl ring, provided when q is 1, R^{s1} and R^{s2} taken together with the carbon atom to which they are attached cannot form a 3-membered cycloalkyl ring;
V is C(═O) or CH₂
Z¹ is —C(═O)— or —SO₂—NH—C(═O)—, wherein the —SO₂— group in —SO₂—NH—C(═O)— is connected to P¹;
R^{x} is absent, C₁₋₁₀alkylene, C₃₋₈cycloalkyl, —(CH₂CH₂O)ₘ₁—C₁₋₁₀alkylene- or C₁₋₁₀-alkylene-(OCH₂CH₂)ₘ₂—;
m1 and m2 are each independently an integer from 1 to 24;
Z² is absent, —C(═O)NH— or —NH—C(═O)—;
R^{y} is absent, C₁₋₁₀alkylene, —(CH₂CH₂O)ₘ₃—C₁₋₁₀alkylene- or C₁₋₁₀alkylene-(OCH₂CH₂)ₘ₄—;
m3 and m4 are each independently an integer from 1 to 24;
Z^{s} is a bifunctional crosslinker bearing a reactive group that is covalently linked to the cytotoxic compound via a disulfide bond or a thioether bond;
J is a moiety comprising a reactive group (preferably, an amine reactive group or a thiol reactive group) that is capable of forming a covalent bond with a cell-binding agent.

3. The compound of claim 1 or 2, wherein R^{1a}, R^{2a}, R^{3a}, R^{4a}, R^{1b}, R^{2b}, R^{3b} and R^{4b} are all H.

4. The compound of any one of claims 1-3, wherein R⁵ is a C₃₋₇alkylene.

5. The compound of claim 4, where R⁵ is —(CH₂)₃—, —(CH₂)₅— or —(CH₂)₇—.

6. The compound of claim 4, wherein R⁵ is —(CH₂)₅—

7. The compound of any one of claims 1-3, wherein R⁵ is represented by the following formula: 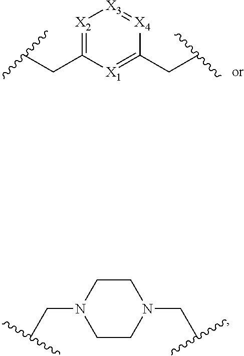
wherein:
X₁, X₂, X₃ and X₄ are each independently N or CR⁶, provided at least one of X₁, X₂, X₃ and X₄ is CR⁶.

8. The compound of claim 7, wherein R⁵ is:

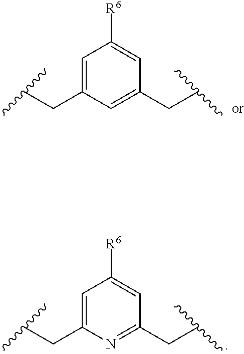

9. The compound of claim 8, wherein R⁵ is: 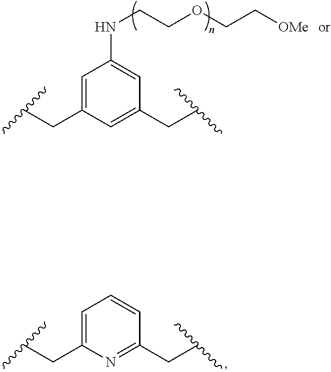
wherein n is an integer from 1 to 8.

10. The compound of claim 9, wherein n is 1, 2, 3 or 4.

11. The compound of claim 2, wherein the compound is represented by the formulae depicted in Table B, or a pharmaceutically acceptable salt thereof.

12. The compound of any one of claims 2-11, wherein Z¹ is —C(═O)—.

13. The compound of any one of claims 2-12, wherein R^{x} is C₁₋₆alkylene, Z² and R^{y} are both absent.

14. The compound of any one of claims 2-12, wherein R^{x} is —(CH₂CH₂O)ₘ₁—C₁₋₆alkylene-; Z² is —NH—C(═O)— or —C(═O)—NH—; R^{y} is C₁₋₆alkylene.

15. The compound of any one of claims 2-12, wherein R^{x} is C₁₋₆alkylene; Z² is —NH—C(═O)— or —C(═O)—NH—; and R^{y} is —(CH₂CH₂O)ₘ₂—C₁₋₆alkylene-.

16. The compound of claim 11, wherein the compound is represented by one of the formulae depicted in Table C, or a pharmaceutically acceptable salt thereof, wherein:
R⁶ is —C(═O)OR^{6a} or NR^{6b}(CH₂CH₂O)ₙCH₂CH₂OR^{6c};
R^{6a}, R^{6b} and R^{ho} are each independently H or C₁₋₄alkyl;
n is an integer from 1 to 8;
R^{a} and R^{b}, for each occurrence, are independently H or C₁₋₄alkyl;
r, r1 and r2 are each independently an integer from 2 to 6, and
s is an integer from 2 to 12.

17. The compound of claim 16, wherein:
R^{6a} and R^{ho} are both Me;
R^{6b} is H;
n is 1, 2, 3, or 4;
R^{a} and R^{b}, for each occurrence, are independently H or Me;
r is 4;
r1 is 4;
r2 is 2;
s is 1, 2, 3 or 4.

18. The compound of any one of claims 2-17, wherein J is —COOR^{d} or a reactive ester represented by COE, wherein R^{d} is H or a C₁₋₄alkyl.

19. The compound of claim 18, wherein J is a reactive ester selected from N-hydroxysuccinimide ester, N-hydroxy sulfosuccinimide ester, nitrophenyl (e.g., 2 or 4-nitrophenyl) ester, dinitrophenyl (e.g., 2,4-dinitrophenyl) ester, sulfo-tetraflurophenyl (e.g., 4-sulfo-2,3,5,6-tetrafluorophenyl) ester, and pentafluorophenyl ester.

20. The compound of claim 18, where J is N-hydroxysuccinimide ester.

21. The compound of any one of claims 2-17, wherein J is

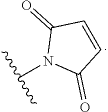 [SMILES: *N1C(=O)C=CC1=O]

22. The compound of any one of claims 2-17, wherein J is —SZ^{s}, wherein Z^{s} is H, SR^{e}, or is selected from the following formulae: 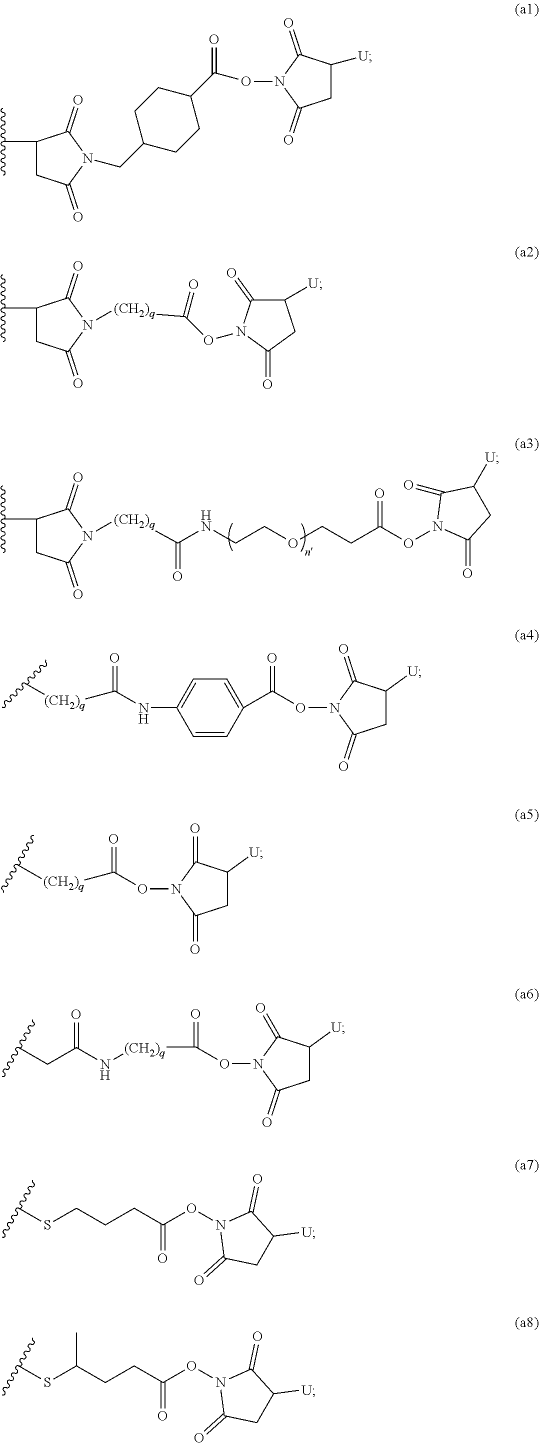 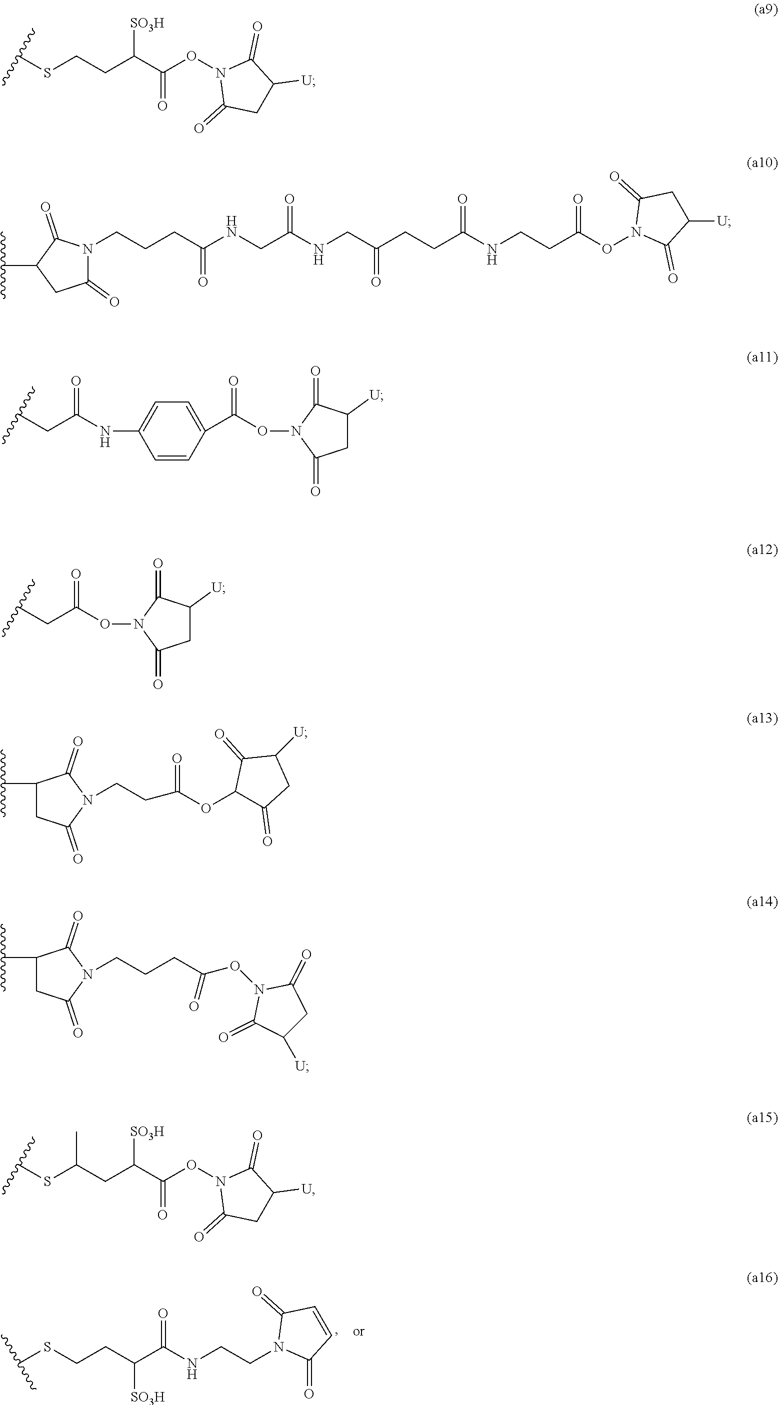 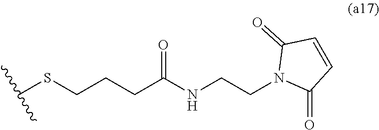
wherein:
q is an integer from 1 to 5;
n′ is an integer from 2 to 6;
U is —H or SO₃H;
R^{e} is a linear or branched alkyl having 1 to 6 carbon atoms or is selected from phenyl, nitrophenyl (e.g., 2 or 4-nitrophenyl), dinitrophenyl (e.g., 2,4-dinitrophenyl), carboxynitrophenyl (e.g., 3-carboxy-4-nitrophenyl), pyridyl or nitropyridyl (e.g., 4-nitropyridyl).

23. The compound of any one of claims 1-22, wherein the double line [symbol] between N and C represents a double bond, X is absent and Y is H.

24. The compound of any one of claims 1-22, wherein the double line [symbol] between N and C represents a single bond, X is H and Y is —SO₃H.

25. The compound of any one of claims 2-24, wherein a1 is an integer from 1 to 7.

26. The compound of claim 25, wherein AA¹-(AA²)ₐ₁ is selected from Gly-Gly-Gly, Ala-Val, Val-Ala, Val-Cit, Val-Lys, Phe-Lys, Lys-Lys, Ala-Lys, Phe-Cit, Leu-Cit, Lle-Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu, β-Ala-Leu-Ala-Leu, Gly-Phe-Leu-Gly, Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Thr-Thr, Thr-Met, Met-Thr, Leu-Ala, Cit-Val, Gln-Val, Ser-Val, Leu-Gln, Gln-Leu, Phe-Arg, Arg-Phe, Tyr-Arg, Arg-Tyr, Phe-Gln, Gln-Phe, Val-Thr, Thr-Val, Met-Tyr, and Tyr-Met.

27. The compound of claim 26, wherein AA¹-(AA²)ₐ₁ is Ala-Ala, L-Ala-L-Ala, Ala-Val, L-Ala-L-Val, Gln-Val, L-Gln-L-Val, Gln-Leu, L-Gln-L-Leu, Ser-Val, or L-Ser-L-Val.

28. The compound of claim 1, wherein the compound is selected from one of the formulae depicted in Table D, or a pharmaceutically acceptable salt thereof, wherein:
R¹⁰⁰ is —OH, —OMe or 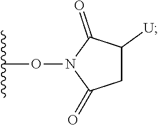
Z^{s} is H, SR^{e}, or is selected from one of the following formulae: 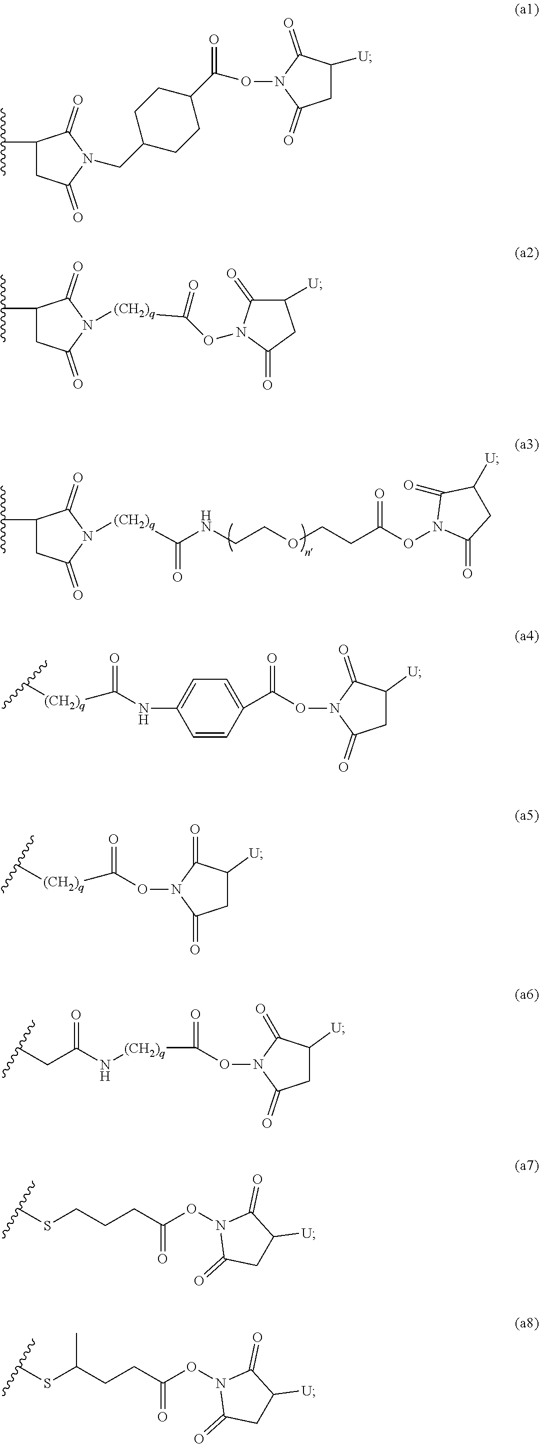 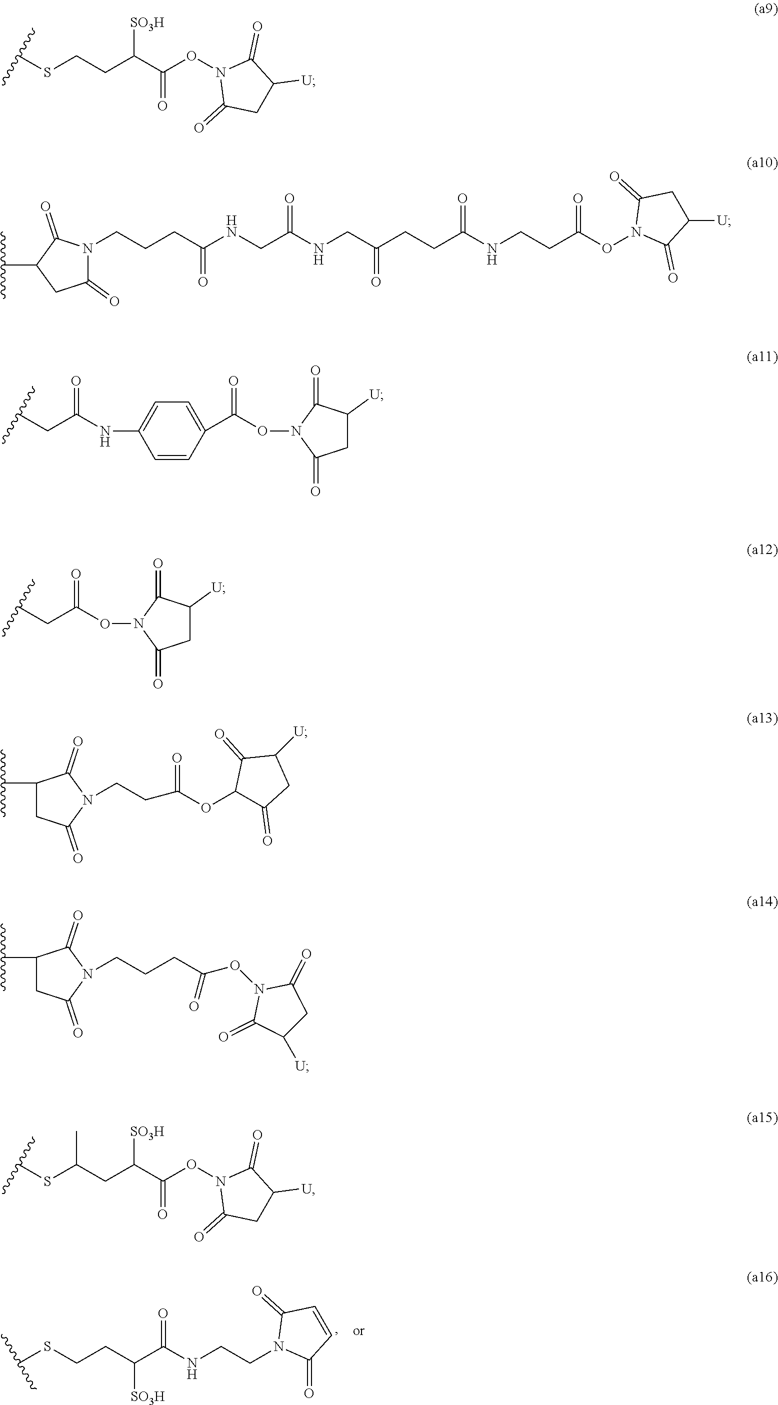 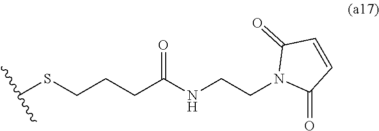
wherein:
q is an integer from 1 to 5;
n′ is an integer from 2 to 6;
U is —H or SO₃H;
R^{e} is a linear or branched alkyl having 1 to 6 carbon atoms or is selected from phenyl, nitrophenyl (e.g., 2 or 4-nitrophenyl), dinitrophenyl (e.g., 2,4-dinitrophenyl), carboxynitrophenyl (e.g., 3-carboxy-4-nitrophenyl), pyridyl or nitropyridyl (e.g., 4-nitropyridyl).

29. The compound of any one of claims 1-28, wherein the pharmaceutically acceptable salt is a sodium or potassium salt.

30. The compound of any one of claims 1-28, wherein the pharmaceutically acceptable salt is a sodium salt.

31. A cell-binding agent-cytotoxic agent conjugate comprising a cell-binding agent (CBA), covalently linked to a cytotoxic agent, wherein the conjugate is represented by the following formula:
CBA-(-Cy)_{w},
or a pharmaceutically acceptable salt thereof, wherein:
CBA is a cell-binding agent;
Cy is a cytotoxic agent represented by the following formula: 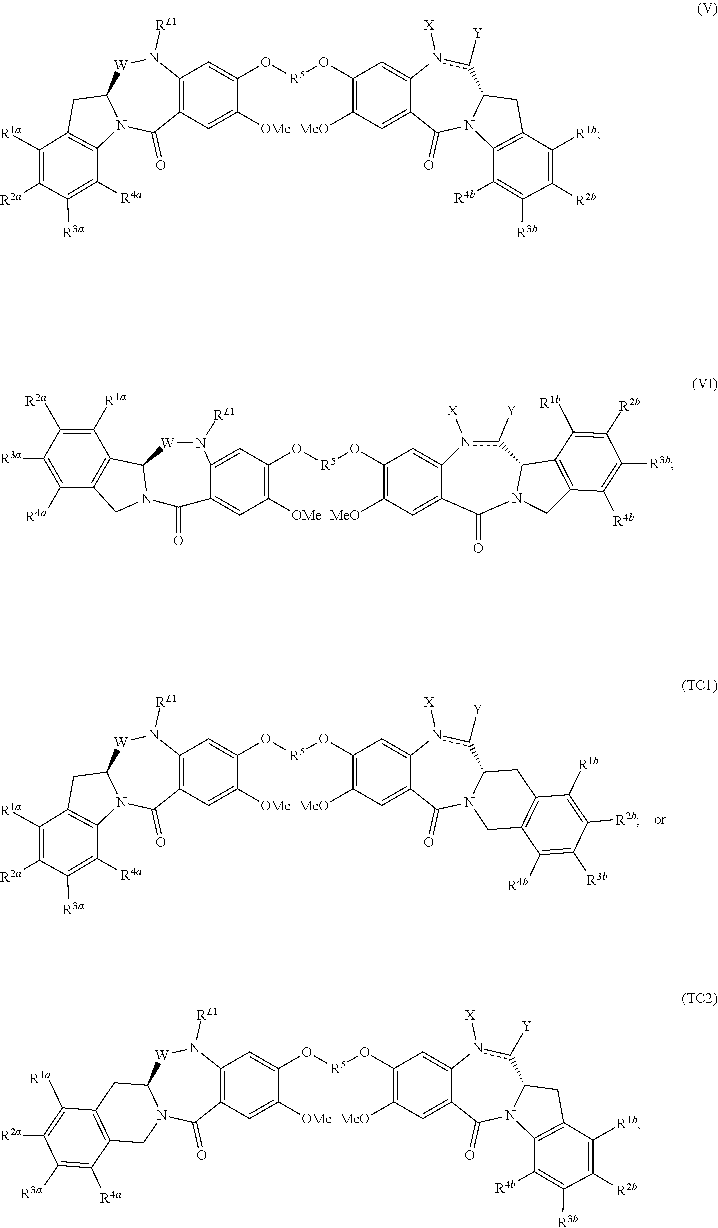
or a pharmaceutically acceptable salt thereof, wherein:
the double line [symbol] between N and C represents a single bond or a double bond, provided that when it is a double bond X is absent and Y is H, or a C₁₋₄alkyl, and when it is a single bond, X is H and Y is —OH or —SO₃H;
W is —C(═O)— or —C(Y′)—;
Y′ is H or C₁₋₄alkyl;
R^{1a}, R^{2a}, R^{3a}, R^{4a}, R^{1b}, R^{2b}, R^{3b} and R^{4b} are each independently selected from the group consisting of H, a C₁₋₁₀alkyl, —(OCH₂CH₂)ₙ—OR^{c}, halogen, —NH(C═NH)NH₂, —OR, —NR′R″, —NO₂, —NR′COR″, —SR, —SOR′, —SO₂R′, —SO₃H, —OSO₃H, —SO₂NR′R″, —CN, —N₃, —COR′, —OCOR′, and —OCONR′R″;
R^{c} is H or a C₁₋₄alkyl;
n is an integer from 1 to 24;
R, for each occurrence, is independently selected from the group consisting of H, —(CH₂CH₂O)ₙ—R^{c}, C₁₋₁₀alkyl, a C₃₋₈cycloalkyl, a 6- to 18-membered aryl, a 5- to 18-membered heteroaryl ring containing one or more heteroatoms independently selected from N, O and S, or a 3- to 18-membered heterocyclic ring containing 1 to 6 heteroatoms independently selected from O, S, N and P;
R′ and R″ are each independently selected from —H, —OH, —OR, —NHR, —NR₂, —COR, a C₁₋₁₀alkyl, a-(CH₂CH₂O)ₙ—R^{c}, and a 3- to 18-membered heterocyclic ring having 1 to 6 heteroatoms independently selected from O, S, N and P;
R⁵ is a C₃₋₁₂alkylene, which chain can be interrupted by one or more groups selected from —O—, —S—, —NH—, —NMe-, benzene ring, a 4 to 7-membered heteroaryl ring and a 4 to 7-membered heterocyclic ring, wherein the benzene, the 4 to 7-membered heteroaryl ring and the 4 to 7-membered heterocyclic ring are substituted with 1 to 4 R⁶;
R⁶ for each occurrence is independently selected from H, C₁₋₁₀alkyl, —(CH₂CH₂O)ₙ—R^{c}, halogen, —NH(C═NH)NH₂, —OR, —NR′R″, —NO₂, —NCO, —NR′COR″, —SR, —SOR′, —SO₂R′, —SO₃H, —OSO₃H, —SO₂NR′R″, —CN, —N₃, —COR′, —OCOR′, and —OCONR′R″; and
R^{L1} is a self-immolative linker covalently linked to the CBA, provided the conjugate of formula (V) is not:

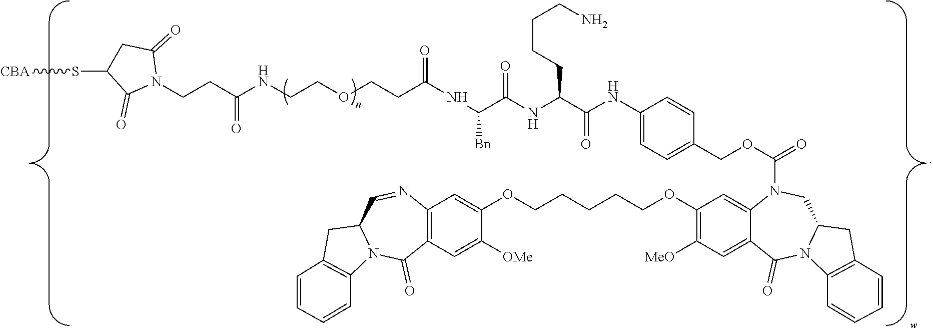

32. The conjugate of claim 31, wherein Cy is represented by one of the formulae depicted in Table E, or a pharmaceutically acceptable salt thereof, wherein:
AA¹ and AA² are each independently an amino acid residues;
a1 is an integer from 1 to 19;
a2 is an integer from 1 to 5;
R^{a} is H or C₁₋₄alkyl;
q is 1, 2, 3 or 4;
R^{s1} and R^{s2} are each independently H or C₁₋₄alkyl, or R^{s1} and R^{s2} taken together with the carbon atom to which they are attached form a 3 to 5-membered cycloalkyl ring, provided when q is 1, R^{s1} and R^{s2} taken together with the carbon atom to which they are attached form a 4 or 5-membered cycloalkyl ring;
V is C(═O) or CH₂;
Z¹ is —C(═O)— or —SO₂—NH—C(═O)—, wherein the —SO₂— group in —SO₂—NH—C(═O)— is connected to P¹;
R^{x} is absent, C₁₋₁₀alkylene, C₃₋₈cycloalkyl, —(CH₂CH₂O)ₘ₁—C₁₋₁₀alkylene- or C₁₋₁₀-alkylene-(OCH₂CH₂)ₘ₂—;
m1 and m2 are each independently an integer from 1 to 24;
Z² is absent, —C(═O)NH— or —NH—C(═O)—;
R^{y} is absent, C₁₋₁₀alkylene, —(CH₂CH₂O)ₘ₃—C₁₋₁₀alkylene- or C₁₋₁₀alkylene-(OCH₂CH₂)ₘ₄—;
m3 and m4 are each independently an integer from 1 to 24;
Z^{s1} is a bifunctional crosslinker that is covalently linked to the CBA and the cytotoxic compound, wherein the crosslinker is covalently linked to the cytotoxic compound via a disulfide bond or a thioether bond; and
J¹ is a moiety formed by reacting an amine reactive group or a thiol reactive group of the cytotoxic agent with an amine group or a thiol group located on CBA.

33. The conjugate of claim 31 or 32, wherein R^{1a}, R^{2a}, R^{3a}, R^{4a}, R^{1b}, R^{2b}, R^{3b} and R^{4b} are all H.

34. The conjugate of any one of claims 31-33, wherein R⁵ is a C₃₋₇alkylene.

35. The conjugate of claim 34, where R⁵ is —(CH₂)₃—, —(CH₂)₅— or —(CH₂)₇—.

36. The conjugate of claim 34, wherein R⁵ is —(CH₂)₅—

37. The conjugate of any one of claims 31-33, wherein R⁵ is represented by the following formula: 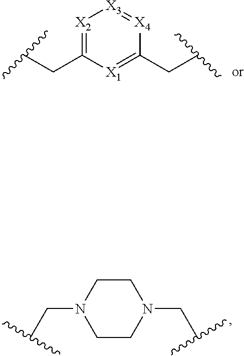
wherein:
X₁, X₂, X₃ and X₄ are each independently N or CR⁶, provided at least one of X₁, X₂, X₃ and X₄ is CR⁶.

38. The conjugate of claim 37, wherein R⁵ is:

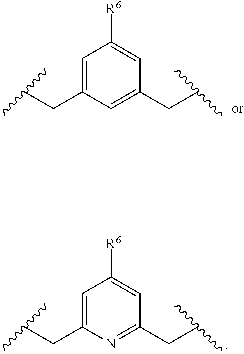

39. The conjugate of claim 38, wherein R⁵ is: 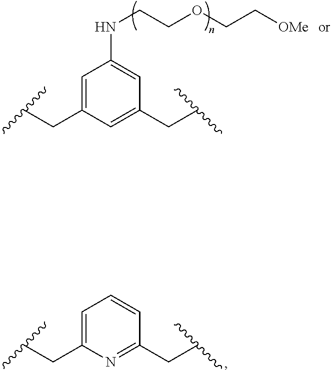
wherein n is an integer from 1 to 8.

40. The conjugate of claim 39, wherein n is 1, 2, 3 or 4.

41. The conjugate of claim 32, wherein Cy is represented by one of the following formulae depicted in Table F, or a pharmaceutically acceptable salt thereof.

42. The conjugate of any one of claims 32-41, wherein Z¹ is —C(═O)—.

43. The conjugate of any one of claims 32-42, wherein R^{x} is C₁₋₆alkylene, Z² and R^{y} are both absent.

44. The conjugate of any one of claims 32-42, wherein R^{x} is —(CH₂CH₂O)ₘ₁—C₁₋₆alkylene-; Z² is —NH—C(═O)— or —C(═O)—NH—; R^{y} is C₁₋₆alkylene.

45. The conjugate of any one of claims 32-42, wherein R^{x} is C₁₋₆alkylene; Z² is —NH—C(═O)— or —C(═O)—NH—; and R^{y} is —(CH₂CH₂O)ₘ₂—C₁₋₆alkylene-.

46. The conjugate of claim 41, wherein Cy is represented by one of the formulae depicted in Table G, or a pharmaceutically acceptable salt thereof, wherein:
R⁶ is —C(═O)OR^{6a} or —NR^{6b}(CH₂CH₂O)ₙCH₂CH₂OR^{6c};
R^{6a}, R^{6b} and R^{ho} are each independently H or C₁₋₄alkyl;
n is an integer from 1 to 8;
R^{a} and R^{b}, for each occurrence, are independently H or C₁₋₄alkyl;
r, r1 and r2 are each independently an integer from 2 to 6, and
s is an integer from 2 to 12.

47. The conjugate of claim 46, wherein:
R^{6a} and R^{6c} are both Me;
R^{6b} is H;
n is 1, 2, 3 or 4;
R^{a} and R^{b}, for each occurrence, are independently H or Me;
r is 4;
r1 is 4;
r2 is 2;
s is 1, 2, 3 or 4.

48. The conjugate of any one of claims 32-47, wherein J¹ is —C(═O)—.

49. The conjugate of any one of claims 32-47, wherein J¹ is 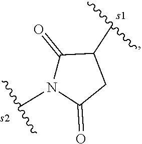
wherein s1 is the site connected to CBA and s2 is the site connected to the rest of the cytotoxic compound.

50. The conjugate of any one of claims 32-47, wherein J¹ is —SZ^{s1}, wherein Z^{s1} is selected from the following formulae: 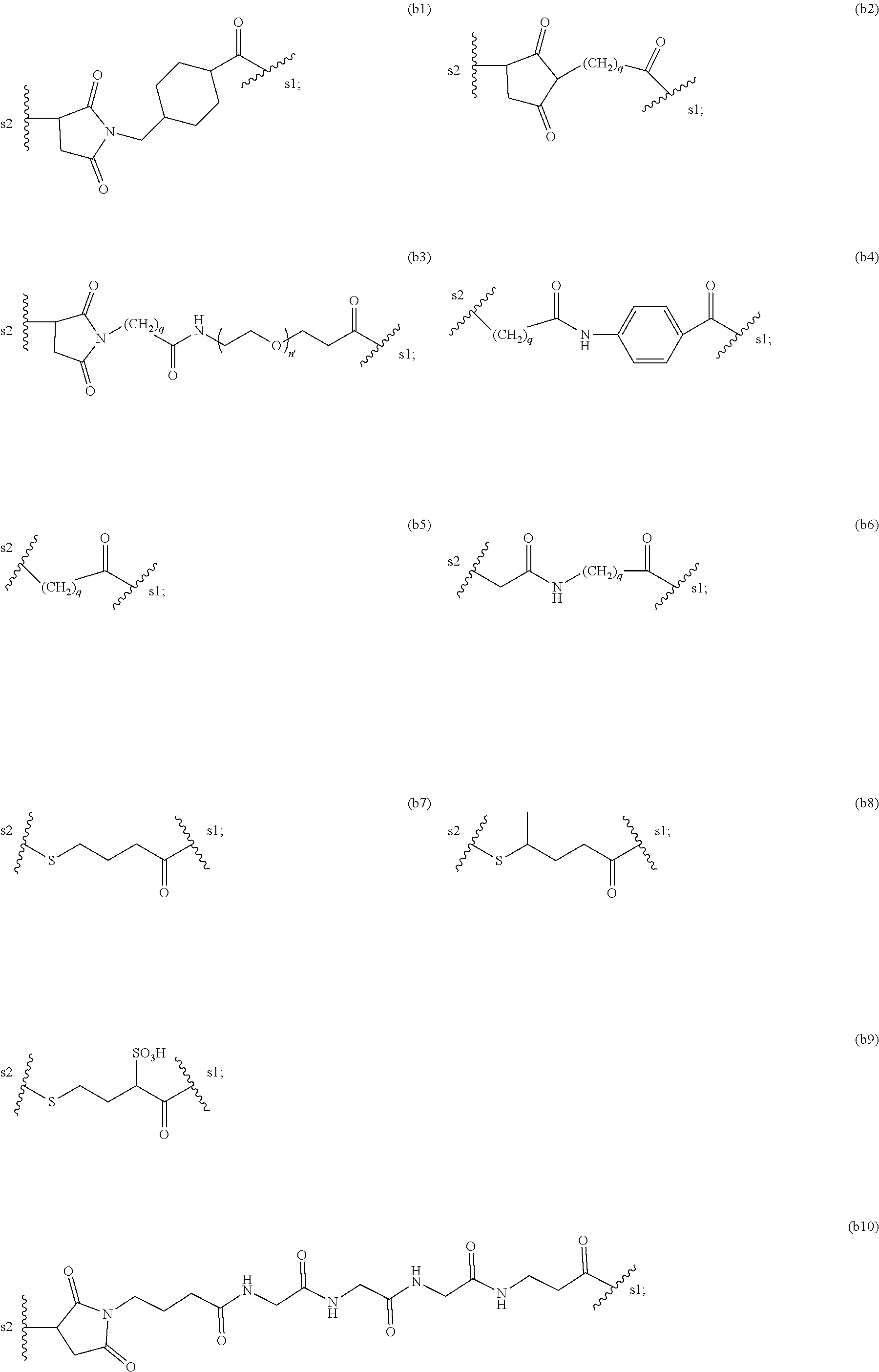 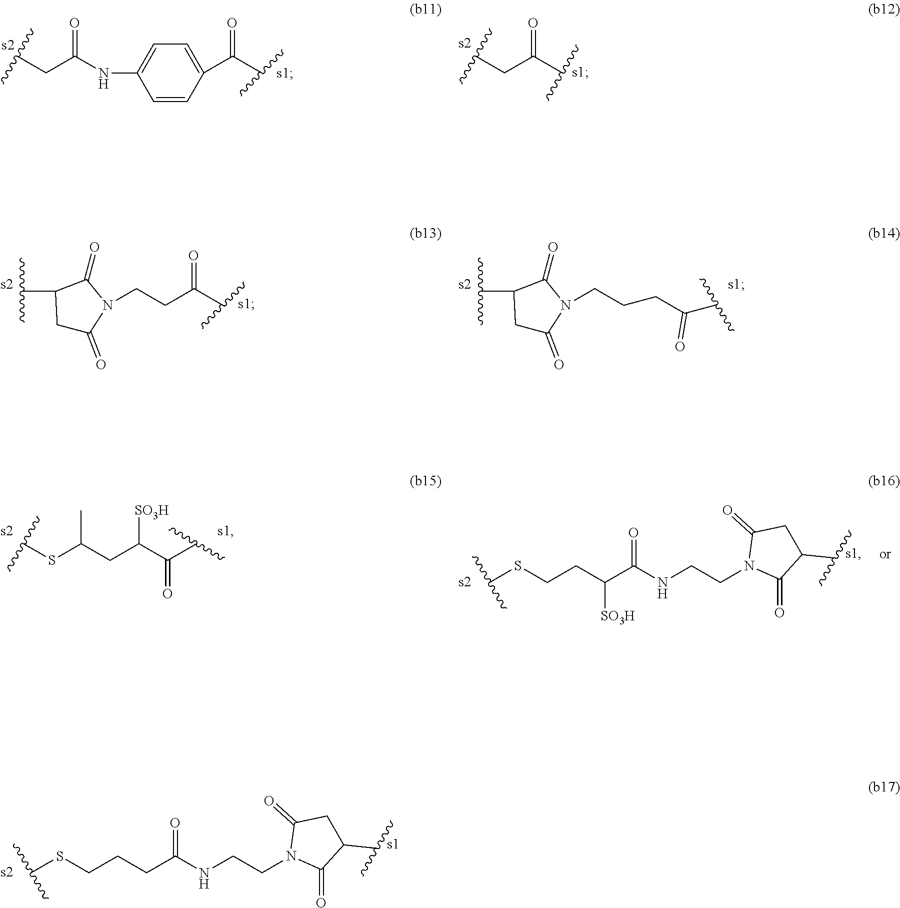
wherein:
q is an integer from 1 to 5;
n′ is an integer from 2 to 6;
s1 is the site connected to CBA; and
s2 is the site connected to the rest of the cytotoxic compound.

51. The conjugate of any one of claims 31-50, wherein the double line [symbol] between N and C represents a double bond, X is absent and Y is H.

52. The conjugate of any one of claims 31-50, wherein the double line [symbol] between N and C represents a single bond, X is H and Y is —SO₃H.

53. The conjugate of any one of claims 32-52, wherein a1 is an integer from 1 to 7.

54. The conjugate of claim 53, wherein AA¹-(AA²)ₐ₁ is selected from Gly-Gly-Gly, Ala-Val, Val-Ala, Val-Cit, Val-Lys, Phe-Lys, Lys-Lys, Ala-Lys, Phe-Cit, Leu-Cit, Lle-Cit, Phe-Ala, Phe-N⁹-tosyl-Arg, Phe-N⁹-nitro-Arg, Phe-Phe-Lys, D-Phe-Phe-Lys, Gly-Phe-Lys, Leu-Ala-Leu, Ile-Ala-Leu, Val-Ala-Val, Ala-Leu-Ala-Leu, β-Ala-Leu-Ala-Leu and Gly-Phe-Leu-Gly, Val-Arg, Arg-Val, Arg-Arg, Val-D-Cit, Val-D-Lys, Val-D-Arg, D-Val-Cit, D-Val-Lys, D-Val-Arg, D-Val-D-Cit, D-Val-D-Lys, D-Val-D-Arg, D-Arg-D-Arg, Ala-Ala, Ala-D-Ala, D-Ala-Ala, D-Ala-D-Ala, Ala-Met, Met-Ala, Thr-Thr, Thr-Met, Met-Thr, Leu-Ala, Cit-Val, Gln-Val, Ser-Val, Val-Gln, Gln-Val, Leu-Gln, Gln-Leu, Phe-Arg, Arg-Phe, Tyr-Arg, Arg-Tyr, Phe-Gln, Gln-Phe, Val-Thr, Thr-Val, Val-Met, Met-Val, Leu-Met, Met-Leu, Met-Tyr, Tyr-Met, Ala-Asn, Asn-Ala, Phe-Met, Met-Phe, Gly-Gly-Arg, and Arg-Gly-Gly.

55. The conjugate of claim 54, wherein AA¹-(AA²)ₐ₁ is Ala-Ala, L-Ala-L-Ala, Ala-Val, L-Ala-L-Val, Gln-Val, L-Gln-L-Val, Gln-Leu, L-Gln-L-Leu, Ser-Val, or L-Ser-L-Val.

56. The conjugate of claim 31, wherein the conjugate is selected from one of the conjugates depicted in Table H, or a pharmaceutically acceptable salt thereof, wherein 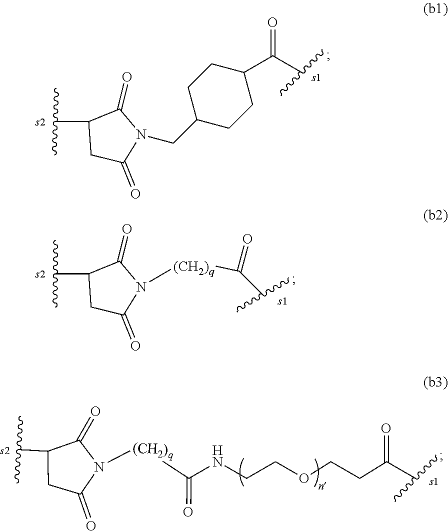
is the cell-binding agent covalently linked to the cytotoxic compound through an amine group located on the CBA; 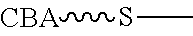
is the cell-binding agent covalently linked to the cytotoxic compound through thiol group located on the CBA; w_{L} is an integer from 1 to 20; and w_{C} is an integer from 1 to 4.

57. The conjugate of any one of claims 31-56, wherein the pharmaceutically acceptable salt is a sodium or potassium salt.

58. The conjugate of any one of claims 31-56, wherein the pharmaceutically acceptable salt is a sodium salt.

59. The conjugate of any one of claims 31-58, wherein the cell-binding agent (CBA) is an antibody, a single chain antibody, an antibody fragment that specifically binds to the target cell, a monoclonal antibody, a single chain monoclonal antibody, or a monoclonal antibody fragment that specifically binds to a target cell, a chimeric antibody, a chimeric antibody fragment that specifically binds to the target cell, a domain antibody, a domain antibody fragment that specifically binds to the target cell, a probody, a nanobody, a lymphokine, a hormone, a vitamin, a growth factor, a colony stimulating factor, or a nutrient-transport molecule.

60. The conjugate of any one of claims 31-59, wherein the cell-binding agent (CBA) binds to target cells selected from tumor cells, virus infected cells, microorganism infected cells, parasite infected cells, autoimmune cells, activated cells, myeloid cells, activated T-cells, B cells, or melanocytes; cells expressing the CA6, CAK1, CD4, CD5, CD6, CD19, CD20, CD22, CD30, CD33, CD37, CD38, CD40, CD44, CD56, CD123, CD138, EpCAM, CanAg, CALLA, CEACAM5, FGFR3, LAMP1, p-cadherin, Her-2 or Her-3 antigens; or cells expressing insulin growth factor receptor, epidermal growth factor receptor, and folate receptor.

61. The conjugate of any one of claims 31-58, wherein the cell-binding agent is an anti-folate receptor antibody or an antibody fragment thereof, an anti-EGFR antibody or an antibody fragment thereof, an anti-CD33 antibody or an antibody fragment thereof, an anti-CD19 antibody or an antibody fragment thereof, an anti-Muc1 antibody or an antibody fragment thereof, or an anti-CD37 antibody or an antibody fragment thereof.

62. A pharmaceutical composition comprising a pharmaceutically acceptable carrier and a conjugate of any one of claims 31-61, or a pharmaceutically acceptable salt thereof.

63. A method of inhibiting abnormal cell growth or treating a proliferative disorder, an autoimmune disorder, destructive bone disorder, infectious disease, viral disease, fibrotic disease, neurodegenerative disorder, pancreatitis or kidney disease in a mammal, comprising administering to said mammal a therapeutically effective amount of a compound of any one of claims 1-30 or a conjugate of any one of claims 31-61, and optionally, a chemotherapeutic agent.

64. The method of claim 63, wherein the method is for treating a cancer.

65. The method of claim 64, wherein the cancer is endometrial cancer, lung cancer (e.g., non-small-cell lung cancer), colorectal cancer, bladder cancer, gastric cancer, pancreatic cancer, renal cell carcinoma, prostate cancer, esophageal cancer, breast cancer, head and neck cancer, uterine cancer, ovarian cancer, liver cancer, cervical cancer, thyroid cancer, testicular cancer, myeloid cancer, melanoma, and lymphoid cancer.

66. The method of claim 64, wherein the cancer is acute myeloid leukemia (AML), chronic myelogenous leukemia (CML), myelodysplastic syndrome (MDS), acute lymphoblastic leukemia (ALL), acute B lymphoblastic leukemia or B-cell acute lymphoblastic leukemia (B-ALL), chronic lymphocytic leukemia (CLL), hairy cell leukemia (HCL), acute promyelocytic leukemia (APL), B-cell chronic lymphoproliferative disease (B-CLPD), atypical chronic lymphocytic leukemia, diffuse large B-cell lymphoma (DLBCL), blastic plasmacytoid dendritic cell neoplasm (BPDCN), non-Hodgkin lymphomas (NHL), mantel cell leukemia (MCL), small lymphocytic lymphoma (SLL), Hodgkin's lymphoma, systemic mastocytosis, and Burkitt's lymphoma.


SEQUENCE LISTING (57 sequences)

SEQ ID NO: 1 - protein, 11 aa, Artificial Sequence
  source: /note=“Description of Artificial Sequence Synthetic peptide”
RASQDINSYLS

SEQ ID NO: 2 - protein, 7 aa, Artificial Sequence
  source: /note=“Description of Artificial Sequence Synthetic peptide”
RVNRLVD

SEQ ID NO: 3 - protein, 9 aa, Artificial Sequence
  source: /note=“Description of Artificial Sequence Synthetic peptide”
LQYDAFPYT

SEQ ID NO: 4 - protein, 5 aa, Artificial Sequence
  source: /note=“Description of Artificial Sequence Synthetic peptide”
SSIMH

SEQ ID NO: 5 - protein, 17 aa, Artificial Sequence
  source: /note=“Description of Artificial Sequence Synthetic peptide”
YIKPYNDGTKYNEKFKG

SEQ ID NO: 6 - protein, 12 aa, Artificial Sequence
  source: /note=“Description of Artificial Sequence Synthetic peptide”
EGGNDYYDTMDY

SEQ ID NO: 7 - protein, 121 aa, Artificial Sequence
  source: /note=“Description of Artificial Sequence Synthetic polypeptide”
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSS

SEQ ID NO: 8 - protein, 450 aa, Artificial Sequence
  source: /note=“Description of Artificial Sequence Synthetic polypeptide”
QVQLVQSGAEVKKPGASVKVSCKASGYIFTSSIMHWVRQAPGQGLEWIGYIKPYNDGTKYNEKFKGRATLTSDRSTSTAYMELSSLRSEDTAVYYCAREGGNDYYDTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 9 - protein, 108 aa, Artificial Sequence
  source: /note=“Description of Artificial Sequence Synthetic polypeptide”
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKR

SEQ ID NO: 10 - protein, 214 aa, Artificial Sequence
  source: /note=“Description of Artificial Sequence Synthetic polypeptide”
DIQMTQSPSSLSASVGDRVTITCRASQDINSYLSWFQQKPGKAPKTLIYRVNRLVDGVPSRFSGSGSGNDYTLTISSLQPEDFATYYCLQYDAFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 17 aa, Artificial Sequence
  source: /note=“Description of Artificial Sequence Synthetic peptide”
KSSQSVFFSSSQKNYLA

SEQ ID NO: 12 - protein, 7 aa, Artificial Sequence
  source: /note=“Description of Artificial Sequence Synthetic peptide”
WASTRES

SEQ ID NO: 13 - protein, 8 aa, Artificial Sequence
  source: /note=“Description of Artificial Sequence Synthetic peptide”
HQYLSSRT

SEQ ID NO: 14 - protein, 5 aa, Artificial Sequence
  source: /note=“Description of Artificial Sequence Synthetic peptide”
SYYIH

SEQ ID NO: 15 - protein, 17 aa, Artificial Sequence
  source: /note=“Description of Artificial Sequence Synthetic peptide”
VIYPGNDDISYNQKFQG

SEQ ID NO: 16 - protein, 9 aa, Artificial Sequence
  source: /note=“Description of Artificial Sequence Synthetic peptide”
EVRLRYFDV

SEQ ID NO: 17 - protein, 118 aa, Artificial Sequence
  source: /note=“Description of Artificial Sequence Synthetic polypeptide”
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 18 - protein, 447 aa, Artificial Sequence
  source: /note=“Description of Artificial Sequence Synthetic polypeptide”
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 113 aa, Artificial Sequence
  source: /note=“Description of Artificial Sequence Synthetic polypeptide”
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 20 - protein, 219 aa, Artificial Sequence
  source: /note=“Description of Artificial Sequence Synthetic polypeptide”
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 15 aa, Artificial Sequence
  source: /note=“Description of Artificial Sequence Synthetic peptide”
KASQSVDYSGDSYMN

SEQ ID NO: 22 - protein, 7 aa, Artificial Sequence
  source: /note=“Description of Artificial Sequence Synthetic peptide”
AASDLES

SEQ ID NO: 23 - protein, 9 aa, Artificial Sequence
  source: /note=“Description of Artificial Sequence Synthetic peptide”
QQSHEDPFT

SEQ ID NO: 24 - protein, 5 aa, Artificial Sequence
  source: /note=“Description of Artificial Sequence Synthetic peptide”
SYWMH

SEQ ID NO: 25 - protein, 17 aa, Artificial Sequence
  source: /note=“Description of Artificial Sequence Synthetic peptide”
EIIPIFGHTNYNEKFKS

SEQ ID NO: 26 - protein, 14 aa, Artificial Sequence
  source: /note=“Description of Artificial Sequence Synthetic peptide”
GGYYYYPRQGFLDY

SEQ ID NO: 27 - protein, 123 aa, Artificial Sequence
  source: /note=“Description of Artificial Sequence Synthetic polypeptide”
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSS

SEQ ID NO: 28 - protein, 111 aa, Artificial Sequence
  source: /note=“Description of Artificial Sequence Synthetic polypeptide”
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 29 - protein, 452 aa, Artificial Sequence
  source: /note=“Description of Artificial Sequence Synthetic polypeptide”
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLCLSPG

SEQ ID NO: 30 - protein, 218 aa, Artificial Sequence
  source: /note=“Description of Artificial Sequence Synthetic polypeptide”
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 16 aa, Artificial Sequence
  source: /note=“Description of Artificial Sequence Synthetic peptide”
RSSRSLLHSDGFTYLY

SEQ ID NO: 32 - protein, 7 aa, Artificial Sequence
  source: /note=“Description of Artificial Sequence Synthetic peptide”
QTSNLAS

SEQ ID NO: 33 - protein, 9 aa, Artificial Sequence
  source: /note=“Description of Artificial Sequence Synthetic peptide”
AQNLELPNT

SEQ ID NO: 34 - protein, 5 aa, Artificial Sequence
  source: /note=“Description of Artificial Sequence Synthetic peptide”
NYYIH

SEQ ID NO: 35 - protein, 17 aa, Artificial Sequence
  source: /note=“Description of Artificial Sequence Synthetic peptide”
WIYPGNVYIQYNEKFKG

SEQ ID NO: 36 - protein, 7 aa, Artificial Sequence
  source: /note=“Description of Artificial Sequence Synthetic peptide”
DGPWFAY

SEQ ID NO: 37 - protein, 116 aa, Artificial Sequence
  source: /note=“Description of Artificial Sequence Synthetic polypeptide”
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGPWFAYWGQGTLVTVSS

SEQ ID NO: 38 - protein, 112 aa, Artificial Sequence
  source: /note=“Description of Artificial Sequence Synthetic polypeptide”
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIK

SEQ ID NO: 39 - protein, 445 aa, Artificial Sequence
  source: /note=“Description of Artificial Sequence Synthetic polypeptide”
QVQLVQSGAEVKKPGASVKVSCKASGYTFTNYYIHWVRQAPGQRLEYIGWIYPGNVYIQYNEKFKGRATLTADKSASTAYMELSSLRSEDTAVYYCARDGPWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 219 aa, Artificial Sequence
  source: /note=“Description of Artificial Sequence Synthetic polypeptide”
DIVLTQTPLSLSVTPGQPASISCRSSRSLLHSDGFTYLYWFLQKPGQSPQLLIYQTSNLASGVPDRFSSSGSGTDFTLKISRVEAEDVGVYYCAQNLELPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 15 aa, Artificial Sequence
  source: /note=“Description of Artificial Sequence Synthetic peptide”
KASQSVSFAGTSLMH

SEQ ID NO: 42 - protein, 7 aa, Artificial Sequence
  source: /note=“Description of Artificial Sequence Synthetic peptide”
RASNLEA

SEQ ID NO: 43 - protein, 9 aa, Artificial Sequence
  source: /note=“Description of Artificial Sequence Synthetic peptide”
QQSREYPYT

SEQ ID NO: 44 - protein, 5 aa, Artificial Sequence
  source: /note=“Description of Artificial Sequence Synthetic peptide”
GYFMN

SEQ ID NO: 45 - protein, 17 aa, Artificial Sequence
  source: /note=“Description of Artificial Sequence Synthetic peptide”
RIHPYDGDTFYNQKFQG

SEQ ID NO: 46 - protein, 9 aa, Artificial Sequence
  source: /note=“Description of Artificial Sequence Synthetic peptide”
YDGSRAMDY

SEQ ID NO: 47 - protein, 10 aa, Artificial Sequence
  source: /note=“Description of Artificial Sequence Synthetic peptide”
GYTFTGYFMN

SEQ ID NO: 48 - protein, 10 aa, Artificial Sequence
  source: /note=“Description of Artificial Sequence Synthetic peptide”
RIHPYDGDTF

SEQ ID NO: 49 - protein, 118 aa, Artificial Sequence
  source: /note=“Description of Artificial Sequence Synthetic polypeptide”
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 50 - protein, 112 aa, Artificial Sequence
  source: /note=“Description of Artificial Sequence Synthetic polypeptide”
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKR

SEQ ID NO: 51 - protein, 112 aa, Artificial Sequence
  source: /note=“Description of Artificial Sequence Synthetic polypeptide”
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKR

SEQ ID NO: 52 - protein, 448 aa, Artificial Sequence
  source: /note=“Description of Artificial Sequence Synthetic polypeptide”
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 218 aa, Artificial Sequence
  source: /note=“Description of Artificial Sequence Synthetic polypeptide”
DIVLTQSPLSLAVSLGQPAIISCKASQSVSFAGTSLMHWYHQKPGQQPRLLIYRASNLEAGVPDRFSGSGSKTDFTLNISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 218 aa, Artificial Sequence
  source: /note=“Description of Artificial Sequence Synthetic polypeptide”
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 4 aa, Artificial Sequence
  source: /note=“Description of Artificial Sequence Synthetic peptide”
ALAL

SEQ ID NO: 56 - protein, 4 aa, Artificial Sequence
  source: /note=“Description of Artificial Sequence Synthetic peptide”
  MOD_RES (1)..(1): Beta-Ala
XLAL

SEQ ID NO: 57 - protein, 4 aa, Artificial Sequence
  source: /note=“Description of Artificial Sequence Synthetic peptide”
GFLG